(19) United States
(12) Patent Application Publication
(10) Pub. No.: US 2026/0091048 A1
(43) Pub. Date: Apr. 2, 2026

(54) GIP RECEPTOR AGONIST COMPOUNDS

(71) Applicant: ELI LILLY AND COMPANY, Indianapolis, IN (US)

(72) Inventors: Jolie Anne BASTIAN, Indianapolis, IN (US); Thomas James BEAUCHAMP, Fishers, IN (US); Douglas Carl BEHENNA, San Juan Capistrano, CA (US); Maria del Pilar BUTELER, San Diego, CA (US); Rachel Katherine CHAMBERS, Indianapolis, IN (US); Jiehao CHEN, Carmel, IN (US); Zhaogen CHEN, Carmel, IN (US); Timothy Barrett DURHAM, Indianapolis, IN (US); Fortune Otuko DZEAGU, San Diego, CA (US); Todd FIELDS, New Palestine, IN (US); Patric James HAHN, Indianapolis, IN (US); Erik James HEMBRE, Indianapolis, IN (US); Youming HUANG, Carmel, IN (US); Steven Lee KUKLISH, Indianapolis, IN (US); Jose Eduardo LOPEZ GARCIA, Fishers, IN (US); Ethan Lindsay MAGNO, San Diego, CA (US); Maxwell Johnson MOORE, San Diego, CA (US); Paul Vincent RUCKER, Carlsbad, CA (US); Daniel Copley SCHMITT, Zionsville, IN (US); Daniel Aaron STRASSFELD, San Diego, CA (US); Lewis Robin VIDLER, Wallington (GB); Gaiying ZHAO, Carmel, IN (US)

(21) Appl. No.: 19/287,515

(22) Filed: Jul. 31, 2025

Related U.S. Application Data
(60) Provisional application No. 63/678,779, filed on Aug. 2, 2024.
(60) Provisional application No. 63/733,738, filed on Dec. 13, 2024.
(60) Provisional application No. 63/783,867, filed on Apr. 4, 2025.

(51) Int. Cl. A61K 31/675; A61K 31/444; A61K 31/4709; A61K 31/4985; A61K 31/501; A61K 31/502; A61K 31/506; A61K 31/5377; A61K 31/55; C07B 59/00; C07D 401/14; C07D 413/14; C07D 417/14; C07D 471/04; C07D 487/04; C07D 495/04; C07D 498/02; C07D 513/04; C07D 519/00; C07F 9/09; C07F 9/50

(52) U.S. Cl. CPC A61K 31/675; A61K 31/444; A61K 31/4709; A61K 31/4985; A61K 31/501; A61K 31/502; A61K 31/506; A61K 31/5377; A61K 31/55; C07B 59/002; C07D 401/14; C07D 413/14; C07D 417/14; C07D 471/04; C07D 487/04; C07D 495/04; C07D 498/02; C07D 513/04; C07D 519/00; C07F 9/098; C07F 9/5004; C07B 2200/05

(57) ABSTRACT

The present disclosure provides compounds of the formula:

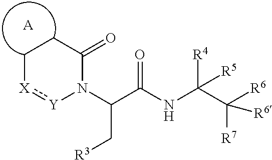

and their pharmaceutically acceptable salts, as well as pharmaceutical compositions comprising these compounds and their use in the treatment of type II diabetes mellitus and obesity.

### FIELD OF THE INVENTION

This invention relates to dihydroisoquinolinone-amide compounds having agonist activity at the human glucose-dependent insulinotropic polypeptide (GIP) receptor, pharmaceutically acceptable salts thereof, pharmaceutical compositions, and therapeutic uses of the compounds. The compounds are useful in the treatment of type 2 diabetes mellitus (T2DM). Also, the compounds are useful in the treatment of obesity.

### BACKGROUND OF THE INVENTION

Over the past several decades, the prevalence of diabetes has continued to rise. T2DM is the most common form of diabetes, accounting for approximately 90% of all diabetes. T2DM is characterized by high blood glucose levels associated mainly with insulin resistance. The current standard of care for T2DM includes diet and exercise, treatment with oral medications, and injectable glucose-lowering drugs, including incretin-based therapies such as Glucagon-like peptide-1 (GLP-1) receptor agonists. When treatment with oral medications and incretin-based therapies are insufficient, insulin treatment is considered. Despite the advances in treatment available today, many patients with T2DM are unable to reach their glycemic control goals. Uncontrolled diabetes leads to several conditions associated with increased morbidity and mortality of patients. There is a need for alternative treatments to enable more patients with T2DM to reach their glycemic treatment goals.

Obesity is a complex medical disorder resulting in excessive accumulation of adipose tissue mass. Today obesity is a global public health concern that is associated with undesired health outcomes and morbidities. Desired treatments for patients with obesity should reduce excess body weight, improve obesity-related co-morbidities, and maintain long-term weight reduction. There is a need for alternative treatment options to induce therapeutic weight loss in patients in need of such treatment.

Both GLP-1 and GIP are incretins, gastrointestinal hormones that regulate blood glucose by enhancing glucose-stimulated insulin secretion. Incretins are also involved in gastric emptying and the regulation of food intake. Incretin-based therapies have provided T2DM and obesity patients with valuable treatment options. Tirzepatide, an agonist having activity at both the GLP-1 receptor (GLP-1R) and GIPR, has been approved as an injectable for the treatment of T2DM (Mounjaro™) and for chronic weight management in adults with obesity (Zepbound™).

Injectable peptide agents, however, may have a number of drawbacks including inconvenience, pain, and the potential for injection site irritation. Therefore, patients often prefer orally administered drugs. Whilst there are a number of GLP-1R agonists in clinical development including, for example, orforglipron (Frias et al., Lancet 2023:402: 472-83), there are currently no such GIPR non-peptide agonists.

There remains a need for alternative treatments for T2DM and obesity. In particular, there is a need for GIPR agonist compounds, especially GIPR agonist compounds which can be administered orally.

### SUMMARY OF THE INVENTION

In a first aspect, there is provided a compound of Formula III

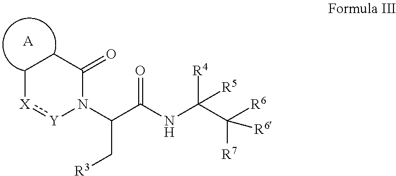

wherein
X is CH₂, OCH₂ or CH₂CH₂, Y is CHR¹⁸ and [symbol] is a single bond, or
X is CH, Y is CR¹⁸ and [symbol] is a double bond, or
X is CHR¹⁴, Y is CHR¹⁵, [symbol] is a single bond and R¹⁴ and R¹⁵ together with the carbons to which
they are attached form a fused cyclopropyl, or
X is N, Y is CH and [symbol] is a double bond, or
X is CH, Y is N and [symbol] is a double bond;
R¹⁸ is H or CH₃;
A is

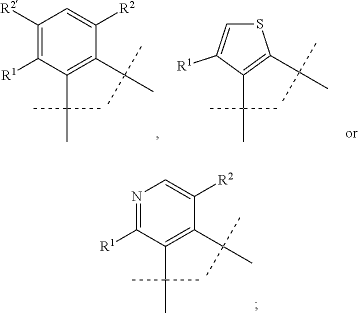

R¹ is an 8-, 9- or 10-membered N-containing bicyclic heterocycle, phenyl or a 5- or 6-membered N-containing heterocycle, wherein the heterocycle or phenyl is optionally substituted with 1 to 3 substituents independently selected from:
   CN,
   halo,
   C₁-C₄alkyl optionally substituted with OCH₃,
   C₁-C₄haloalkyl,
   C₁-C₄alkoxy,
   C₁-C₄haloalkoxy,
   CD₃,
   oxo,
   phenyl optionally substituted with 1 or 2 substituents independently selected from halo and OH,
   benzyl optionally substituted with OCH₃,
   NHC(O)R¹⁶, and
   a 9-membered N-containing bicyclic heterocycle optionally substituted with CH₃;
R¹⁶ is NHC₁-C₄alkyl, C₃-C₆cycloalkyl, NHC₃-C₆cycloalkyl, pyridyl optionally substituted with halo or CH₃, or phenyl optionally substituted with halo;
R² and R^{2′} are independently H, halo or OCH₃; R³ is:
   i) a 5- or 6-membered N-containing heteroaryl optionally substituted with 1 or 2 substituents selected from: CH₃, CD₃, CHF₂, OH and CH₂CN, or
   ii) an 8- or 9-membered N-containing bicyclic heterocycle optionally substituted with 1 or 2 substituents selected from oxo, CH₃ and halo;
R⁴ and R⁵ are each CH₃, or together form a cyclopropyl, cyclobutyl, oxetane, tetrahydrofuran, pyrrolidine or piperidine, wherein the cyclopropyl or cyclobutyl is optionally substituted with 1 or 2 halo and wherein the pyrrolidine or piperidine is optionally substituted with CH₃;
R⁶ is H, D, OH, CH₃, CO₂R¹⁰, or

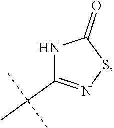

wherein R¹⁰ is H or C₁-C₄alkyl optionally substituted with OC(O)C₁-C₄alkyl or morpholine, R^{6′} is H, and R⁷ is a 5- or 6-membered N-containing heteroaryl, 9-membered N-containing heteroaryl or phenyl, wherein the heteroaryl or phenyl is optionally substituted with R⁸ or with R⁸ and R⁹, or
R⁶ is D, R^{6′} is D and R⁷ is a 5- or 6-membered N-containing heteroaryl, 9-membered N-containing heteroaryl or phenyl, wherein the heteroaryl or phenyl is optionally substituted with R⁸ or with R⁸ and R⁹, or
R⁶ and R⁷ together form a 9-membered N-containing bicyclic heterocycle optionally substituted with 1 to 3 substituents independently selected from: CH₃, CF₃ and oxo, and R^{6′} is H;
R⁸ is CF₃, CF₂H, halo, cyclopropyl, CH₃ or H;
R⁹ is:
   i) a 4-, 5- or 6-membered heterocycle optionally substituted with 1 to 4 substituents independently selected from:
      halo,
      OH
      C₁-C₄alkoxy,
      SO₂NH₂,
      a 4- to 6-membered heterocycle optionally substituted with CH₃ or oxo,
      CN,
      C₃-C₆cycloalkyl,
      CD₃,
      C₁-C₄haloalkyl,
      CR¹²R¹³OP(O)(OH)₂
      oxo,
      C₁-C₄haloalkoxy,
      C(O)NR¹²R¹³,
      (CH₂)ₘP(O)(R¹⁷)₂, and
      C₁-C₄alkyl, wherein the C₁-C₄alkyl is optionally substituted with OH, OCH₃,
      NR¹²R¹³, C(O)NR¹²R¹³, CN, a 4- to 6-membered heterocycle or C₃-C₄cycloalkyl optionally substituted with halo,
   ii) a 9- or 10-membered N-containing bicyclic heterocycle optionally substituted with 1 or 2 substituents independently selected from CN, oxo, C(O)O(CH₃)₃, OH, CH₃, and halo,
   iii) C₁-C₄alkoxy,
   iv) C₃-C₅cycloalkyl,
   v) halo,
   vi) (CH₂)ₙC(O)R¹¹, wherein n is 0 or 1, R¹¹ is OCH₃, NR¹²R¹³ or a 4- to 6-membered heterocycle,
   vii) C₁-C₄alkyl optionally substituted with a 4- to 6-membered heterocycle, phenyl, NHS(O)₂(CH₃) or NR¹²R¹³, which heterocycle or phenyl is optionally substituted with CH₃,
   viii) CN,
   ix) phenyl optionally substituted with 1 to 3 substituents independently selected from C(O)OH, (CH₂)ₘP(O)(R¹⁷)₂, NR¹²R¹³ and C₁-C₄alkyl, wherein the C₁-C₄alkyl is optionally substituted with OH, or
   x) (CH₂)ₘP(O)(R¹⁷)₂;
R¹² and R¹³ are independently H or CH₃;
each R¹⁷ is independently C₁-C₄alkyl; and
m is 0 or 1;
or a pharmaceutically acceptable salt thereof.

In a second aspect, there is provided a pharmaceutical composition comprising a compound of Formula III, or a pharmaceutically acceptable salt thereof, and at least one pharmaceutically acceptable carrier, diluent, or excipient.

In a third aspect, there is provided a method of treating type II diabetes mellitus in a patient comprising administering to the patient a therapeutically effective amount of a compound Formula III, or a pharmaceutically acceptable salt thereof.

In a fourth aspect, there is provided a method of treating obesity in a patient comprising administering to the patient a therapeutically effective amount of a compound of Formula III, or a pharmaceutically acceptable salt thereof.

In a fifth aspect, there is provided a compound of Formula III, or a pharmaceutically acceptable salt thereof, for use in therapy.

In a sixth aspect, there is provided a compound of Formula III, or a pharmaceutically acceptable salt thereof, for use in the treatment of type II diabetes mellitus.

In a seventh aspect, there is provided a compound Formula III, or a pharmaceutically acceptable salt thereof, for use in treating obesity.

### DETAILED DESCRIPTION OF THE INVENTION

In an embodiment of Formula III, there is a provided a compound of Formula IIIa:

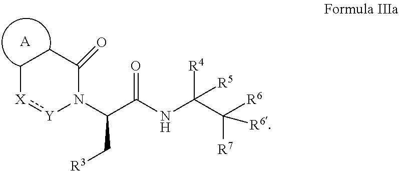

In an embodiment of Formula III, there is provided a compound wherein:
X is CH₂, OCH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl,
X is N, Y is CH and [symbol] is a double bond, or
X is CH, Y is N and [symbol] is a double bond;
A is

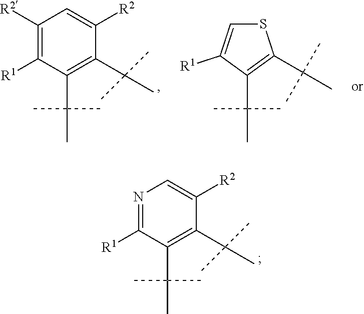

R¹ is a 8-, 9- or 10-membered N-containing bicyclic heterocycle, phenyl or a 5- or 6-membered N-containing heteroaryl, wherein the heterocycle, phenyl or heteroaryl is optionally substituted with 1 to 3 substituents independently selected from:
   CN,
   halo,
   C₁-C₄alkyl optionally substituted with OCH₃,
   C₁-C₄haloalkyl,
   C₁-C₄alkoxy,
   C₁-C₄haloalkoxy,
   CD₃,
   oxo,
   phenyl optionally substituted with 1 or 2 substituents independently selected from halo and OH,
   benzyl optionally substituted with OCH₃,
   NHC(O)pyridyl, wherein the pyridyl is optionally substituted with halo, and a 9-membered N-containing bicyclic heterocycle optionally substituted with CH₃;
R² and R^{2′} are independently H, halo or OCH₃;

R³ is a 5- or 6-membered N-containing heteroaryl optionally substituted with 1 or 2 substituents selected from: CH₃, CD₃, CHF₂, OH and CH₂CN, or a 9-membered N-containing bicyclic heterocycle optionally substituted with 1 or 2 substituents selected from oxo and CH₃;
R⁴ and R⁵ are each CH₃ or together form a cyclopropyl;
R⁶ is H, D, OH, CH₃, CO₂R¹⁰, or

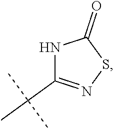

wherein R¹⁰ is H or C₁-C₄alkyl optionally substituted with OC(O)C₁-C₄alkyl or morpholine, and R⁶ is H, or
R⁶ is D and R^{6′} is D;
R⁷ is a 5- or 6-membered N-containing heteroaryl or phenyl, wherein the heteroaryl or phenyl is optionally substituted with R⁸ or with R⁸ and R⁹, or wherein the phenyl is optionally substituted with 1 or 2 substituents selected from: halo, CF₃, and pyrazine optionally substituted with CF₂H or OCH₃;
R⁸ is CF₃, CF₂H, halo or cyclopropyl;
R⁹ is:
   i) a 5- or 6-membered heterocycle optionally substituted with 1 to 3 substituents independently selected from:
      halo,
      OH,
      C₁-C₄alkoxy,
      SO₂NH₂,
      a 4- to 6-membered heterocycle optionally substituted with CH₃,
      CN,
      C₃-C₆cycloalkyl,
      CD₃,
      C₁-C₄haloalkyl,
      CR¹²R¹³OP(O)(OH)₂
      oxo, and
      C₁-C₄alkyl, wherein the C₁-C₄alkyl is optionally substituted with OH, OCH₃,
      N(CH₃)₂, a 4- to 6-membered heterocycle or C₃-C₄cycloalkyl optionally substituted with halo,
   ii) a 9-membered N-containing bicyclic heterocycle optionally substituted with CN, oxo, C(O)O(CH₃)₃ or OH,
   iii) C₁-C₄alkoxy,
   iv) C₃-C₅cycloalkyl,
   v) halo,
   vi) (CH₂)ₘC(O)R¹¹, wherein n is 0 or 1, R¹¹ is OCH₃, NR¹²R¹³ or a 4- to 6-membered heterocycle,
   vii) C₁-C₄alkyl optionally substituted with a 4- to 6-membered heterocycle or phenyl, which heterocycle or phenyl is optionally substituted with CH₃,
   viii) CN, or
   ix) phenyl optionally substituted with C(O)OH; and
R¹² and R¹³ are independently H or CH₃.

In an embodiment of Formula III, R⁷ is a 5- or 6-membered N-containing heteroaryl, 9-membered N-containing heteroaryl or phenyl, wherein the heteroaryl or phenyl is substituted with R⁸ and R⁹, and wherein R⁹ is a 4-, 5- or 6-membered heterocycle substituted with CR¹²R¹³OP(O)(OH)₂ and optionally further substituted with 1 to 3 substituents independently selected from:
halo,
OH,
C₁-C₄alkoxy,
SO₂NH₂,
a 4- to 6-membered heterocycle optionally substituted with CH₃ or oxo,
CN,
C₃-C₆cycloalkyl,
CD₃,
C₁-C₄haloalkyl,
oxo,
C₁-C₄haloalkoxy,
C(O)NR¹²R¹³,
(CH₂)ₘP(O)(R¹⁷)₂, and
C₁-C₄alkyl, wherein the C₁-C₄alkyl is optionally substituted with OH, OCH₃,
NR¹²R¹³, C(O)NR¹²R¹³, CN, a 4- to 6-membered heterocycle or C₃-C₄cycloalkyl optionally substituted with halo.

In an embodiment of Formula III, R⁷ is a 5- or 6-membered N-containing heteroaryl or phenyl, wherein the heteroaryl or phenyl is optionally substituted with R⁸ and R⁹, and wherein R⁹ is a 5- or 6-membered heterocycle substituted with CR¹²R¹³OP(O)(OH)₂ and optionally further substituted with 1 or 2 substituents independently selected from:
halo,
OH,
C₁-C₄alkoxy,
SO₂NH₂,
a 4- to 6-membered heterocycle optionally substituted with CH₃,
CN,
C₃-C₆cycloalkyl,
CD₃,
C₁-C₄haloalkyl,
oxo, and
C₁-C₄alkyl, wherein the C₁-C₄alkyl is optionally substituted with OH, OCH₃,
N(CH₃)₂, a 4- to 6-membered heterocycle or C₃-C₄cycloalkyl optionally substituted with halo.

In an alternate embodiment of Formula III, R⁷ is a 5- or 6-membered N-containing heteroaryl, 9-membered N-containing heteroaryl or phenyl, wherein the heteroaryl or phenyl is optionally substituted with R⁸ or with R⁸ and R⁹; R⁹ is:
i) a 4-, 5- or 6-membered heterocycle optionally substituted with 1 to 4 substituents independently selected from:
   halo,
   OH,
   C₁-C₄alkoxy,
   SO₂NH₂,
   a 4- to 6-membered heterocycle optionally substituted with CH₃ or oxo,
   CN,
   C₃-C₆cycloalkyl,
   CD₃,
   C₁-Cahaloalkyl,
   oxo,
   C₁-C₄haloalkoxy,
   C(O)NR¹²R¹³,
   (CH₂)ₘP(O)(R¹⁷)₂, and
   C₁-C₄alkyl, wherein the C₁-C₄alkyl is optionally substituted with OH, OCH₃, NR¹²R¹³, C(O)NR¹²R¹³, CN, a 4- to 6-membered heterocycle or C₃-C₄cycloalkyl optionally substituted with halo,
ii) a 9- or 10-membered N-containing bicyclic heterocycle optionally substituted with 1 or 2 substituents independently selected from CN, oxo, C(O)O(CH₃)₃, OH, CH₃, and halo,
iii) C₁-C₄alkoxy,
iv) C₃-C₅cycloalkyl,
v) halo,
vi) (CH₂)ₙC(O)R¹¹, wherein n is 0 or 1, R¹¹ is OCH₃, NR¹²R¹³ or a 4- to 6-membered heterocycle,
vii) C₁-C₄alkyl optionally substituted with a 4- to 6-membered heterocycle, phenyl, NHS(O)₂(CH₃) or NR¹²R¹³, which heterocycle or phenyl is optionally substituted with CH₃,
viii) CN,
ix) phenyl optionally substituted with 1 to 3 substituents independently selected from C(O)OH, (CH₂)ₘP(O)(R¹⁷)₂, NR¹²R¹³ and C₁-C₄alkyl, wherein the C₁-C₄alkyl is optionally substituted with OH; or
x) (CH₂)ₘP(O)(R¹⁷)₂.

In an alternate embodiment of Formula III, R⁷ is a 5- or 6-membered N-containing heteroaryl or phenyl, wherein the heteroaryl or phenyl is optionally substituted with R⁸ or with R⁸ and R⁹, or wherein the phenyl is optionally substituted with 1 or 2 substituents selected from: halo, CF₃, and pyrazine optionally substituted with CF₂H or OCH₃; R⁸ is CF₃, CF₂H, halo or cyclopropyl; R⁹ is:
i) a 5- or 6-membered heterocycle optionally substituted with 1 to 3 substituents independently selected from:
   halo,
   OH,
   C₁-C₄alkoxy,
   SO₂NH₂,
   a 4- to 6-membered heterocycle optionally substituted with CH₃,
   CN,
   C₃-C₆cycloalkyl,
   CD₃,
   C₁-C₄haloalkyl,
   oxo, and
   C₁-C₄alkyl, wherein the C₁-C₄alkyl is optionally substituted with OH, OCH₃,
   N(CH₃)₂, a 4- to 6-membered heterocycle or C₃-C₄cycloalkyl optionally substituted with halo,
ii) a 9-membered N-containing bicyclic heterocycle optionally substituted with CN, oxo, C(O)O(CH₃)₃ or OH,
iii) C₁-C₄alkoxy,
iv) C₃-C₅cycloalkyl,
v) halo,
vi) (CH₂)ₙC(O)R¹¹, wherein n is 0 or 1, R¹¹ is OCH₃, NR¹²R¹³ or a 4- to 6-membered heterocycle,
vii) C₁-C₄alkyl optionally substituted with a 4- to 6-membered heterocycle or phenyl, which heterocycle or phenyl is optionally substituted with CH₃,
viii) CN, or
ix) phenyl optionally substituted with C(O)OH.

In an embodiment of Formula III, A is

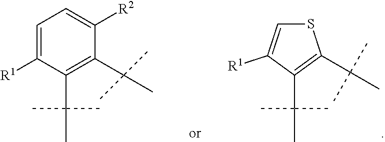

In an embodiment of Formula III, R², when present, is F and R^{2′}, when present, is H or F. In an embodiment of Formula III, R², when present, is F.

In an embodiment of Formula III,
X is CH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond, or
X is CH, Y is N and [symbol] is a double bond.

In an embodiment of Formula III,
X is CH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond.

In an embodiment of Formula III, R⁴ and R⁵ are each CH₃.

In an embodiment of Formula III, R¹ is an 8- or 9-membered N-containing bicyclic heterocycle, phenyl or a 6-membered N-containing heterocycle, wherein the heterocycle or phenyl is optionally substituted with 1 to 3 substituents independently selected from:
CN,
halo,
C₁-C₄alkyl,
C₁-C₄haloalkyl,
C₁-C₄alkoxy,
C₁-C₄haloalkoxy,
CD₃,
oxo,
benzyl optionally substituted with OCH₃, and
a 9-membered N-containing bicyclic heterocycle optionally substituted with CH₃.

In an embodiment of Formula III, R³ is a 5- or 6-membered N-containing heteroaryl optionally substituted with CH₃, CD₃ or CHF₂, or a 9-membered N-containing bicyclic heterocycle optionally substituted with 1 or 2 substituents selected from oxo and CH₃.

In an embodiment of Formula III, R⁶ is H, OH, CO₂R¹⁰, or

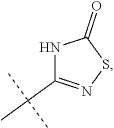

and R⁶ is H. In an embodiment of Formula III, R⁶ is H, CO₂R¹⁰, or

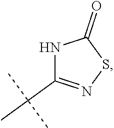

and R^{6′} is H. In an alternate embodiment of Formula III, R⁶ is D and R^{6′} is D.

In an alternate embodiment, R⁶ and R⁷ together form a 9-membered N-containing bicyclic heterocycle optionally substituted with 1 to 3 substituents independently selected from: CH₃, CF₃ and oxo, and R⁶ is H.

In an embodiment of Formula III, R⁷ is a 5- or 6-membered N-containing heteroaryl, 9-membered N-containing heteroaryl or phenyl, wherein the heteroaryl or phenyl is optionally substituted with R⁸ or with R⁸ and R⁹;
R⁸ is CF₃, halo, cyclopropyl, CH₃ or H;
R⁹ is:
   i) a 4-, 5- or 6-membered heterocycle optionally substituted with 1 to 3 substituents independently selected from:
      halo,
      OH,
      C₁-C₄alkoxy,
      a 4- to 6-membered heterocycle,
      CN,
      C₃-C₆cycloalkyl,
      CD₃,
      C₁-C₄haloalkyl,
      CR¹²R¹³OP(O)(OH)₂
      oxo, and
      C₁-C₄alkyl, wherein the C₁-C₄alkyl is optionally substituted with OH, NR¹²R¹³, or a 4- to 6-membered heterocycle,
   ii) a 9-membered N-containing bicyclic heterocycle,
   iii) C₁-C₄alkoxy,
   iv) C₃-C₅cycloalkyl,
   v) halo,
   vi) (CH₂)ₙC(O)R¹¹, wherein n is 0 or 1, R¹¹ is NR¹²R¹³ or a 4- to 6-membered heterocycle,
   vii) C₁-C₄alkyl optionally substituted with NHS(O)₂(CH₃), NR¹²R¹³ or phenyl, which phenyl is optionally substituted with CH₃,
   viii) CN,
   ix) phenyl optionally substituted with 1 or 2 substituents independently selected from C(O)OH, (CH₂)ₘP(O)(R¹⁷)₂ and NR¹²R¹³, or
   x) (CH₂)ₘP(O)(R¹⁷)₂;
R¹² and R¹³ are independently H or CH₃;
each R¹⁷ is independently C₁-C₄alkyl; and
m is 0 or 1.

In an embodiment of Formula III, R⁷ is a 5- or 6-membered N-containing heteroaryl or phenyl, wherein the heteroaryl or phenyl is optionally substituted with R⁸ or with R⁸ and R⁹; R⁸ is CF₃, halo or cyclopropyl;
R⁹ is:
   i) a 5- or 6-membered heterocycle optionally substituted with 1 to 3 substituents independently selected from:
      halo,
      OH,
      C₁-C₄alkoxy,
      a 4- to 6-membered heterocycle,
      CN,
      C₃-C₆cycloalkyl,
      CD₃,
      C₁-C₄haloalkyl,
      CR¹²R¹³OP(O)(OH)₂
      oxo, and
      C₁-C₄alkyl, wherein the C₁-C₄alkyl is optionally substituted with OH, N(CH₃)₂, or a 4- to 6-membered heterocycle,
   ii) a 9-membered N-containing bicyclic heterocycle,
   iii) C₁-C₄alkoxy,
   iv) C₃-C₅cycloalkyl,
   v) halo,
   vi) (CH₂)ₙC(O)R¹¹, wherein n is 0 or 1, R¹¹ is NR¹²R¹³ or a 4- to 6-membered heterocycle,
   vii) C₁-C₄alkyl, or
   viii) CN; and
R¹² and R¹³ are independently H or CH₃.

In an embodiment, there is a provided a compound of Formula V:

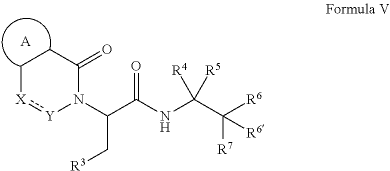

wherein
X is CH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond, or
X is CH, Y is N and [symbol] is a double bond;
A is

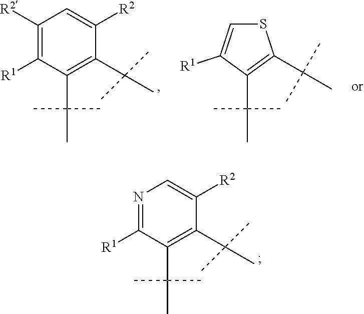

R¹ is an 8- or 9-membered N-containing bicyclic heterocycle, phenyl or a 6-membered N-containing heterocycle, wherein the heterocycle or phenyl is optionally substituted with 1 to 3 substituents independently selected from:
   CN,
   halo,
   C₁-C₄alkyl,
   C₁-C₄haloalkyl,
   C₁-C₄alkoxy,
   C₁-C₄haloalkoxy,
   CD₃,
   oxo,
   benzyl optionally substituted with OCH₃, and
   a 9-membered N-containing bicyclic heterocycle optionally substituted with CH₃;
R² is F;
R^{2′} is H or F;
R³ is a 5- or 6-membered N-containing heteroaryl optionally substituted with CH₃, CD₃ or CHF₂, or a 9-membered N-containing bicyclic heterocycle optionally substituted with 1 or 2 substituents selected from oxo and CH₃;
R⁶ is H, OH, CO₂R¹⁰, or

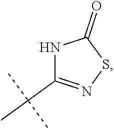

wherein R¹⁰ is H or C₁-C₄alkyl optionally substituted with OC(O)C₁-C₄alkyl or morpholine, R^{6′} is H, and R⁷ is a 5- or 6-membered N-containing heteroaryl, 9-membered N-containing heteroaryl or phenyl, wherein the heteroaryl or phenyl is optionally substituted with R⁸ or with R⁸ and R⁹, or
R⁶ is D, R^{6′} is D and R⁷ is a 5- or 6-membered N-containing heteroaryl, 9-membered N-containing heteroaryl or phenyl, wherein the heteroaryl or phenyl is optionally substituted with R⁸ or with R⁸ and R⁹, or
or R⁶ and R⁷ together form a 9-membered N-containing bicyclic heterocycle optionally substituted with 1 to 3 substituents independently selected from: CH₃, CF₃ and oxo, and R^{6′} is H;
R⁸ is CF₃, halo, cyclopropyl, CH₃ or H;
R⁹ is:
   i) a 4-, 5- or 6-membered heterocycle optionally substituted with 1 to 3 substituents independently selected from:
      halo,
      OH,
      C₁-C₄alkoxy,
      a 4- to 6-membered heterocycle,
      CN,
      C₃-C₆cycloalkyl,
      CD₃,
      C₁-C₄haloalkyl,
      CR¹²R¹³OP(O)(OH)₂
      oxo, and
      C₁-C₄alkyl, wherein the C₁-C₄alkyl is optionally substituted with OH, NR¹²R¹³, or a 4- to 6-membered heterocycle,
   ii) a 9-membered N-containing bicyclic heterocycle,
   iii) C₁-C₄alkoxy,
   iv) C₃-C₅cycloalkyl,
   v) halo,
   vi) (CH₂)ₙC(O)R¹¹, wherein n is 0 or 1, R¹¹ is NR¹²R¹³ or a 4- to 6-membered heterocycle,
   vii) C₁-C₄alkyl optionally substituted with NHS(O)₂(CH₃), NR¹²R¹³ or phenyl, which phenyl is optionally substituted with CH₃,
   viii) CN;
   ix) phenyl optionally substituted with 1 or 2 substituents independently selected from C(O)OH, (CH₂)ₘP(O)(R¹⁷)₂ and NR¹²R¹³, or
   x) (CH₂)ₘP(O)(R¹⁷)₂;
R¹² and R¹³ are independently H or CH₃;
each R¹⁷ is independently C₁-C₄alkyl; and
m is 0 or 1,
or a pharmaceutically acceptable salt thereof.

In an embodiment of Formula V, there is a provided a compound of Formula Va:

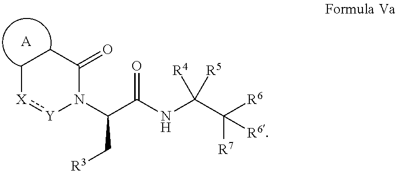

In an embodiment, there is a provided a compound of Formula IV:

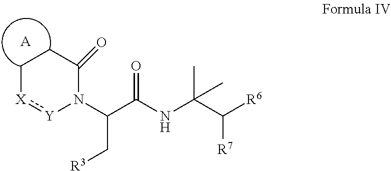

wherein
X is CH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond;
A is

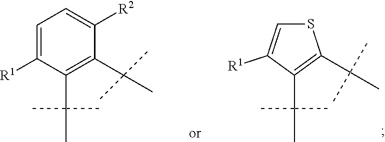

R¹ is an 8- or 9-membered N-containing bicyclic heterocycle, phenyl or a 6-membered N-containing heteroaryl, wherein the heterocycle, phenyl or heteroaryl is optionally substituted with 1 to 3 substituents independently selected from:
   CN,
   halo,
   C₁-C₄alkyl,
   C₁-C₄haloalkyl,
   C₁-C₄alkoxy,
   C₁-C₄haloalkoxy,
   CD₃,
   oxo,
   benzyl optionally substituted with OCH₃, and
   a 9-membered N-containing bicyclic heterocycle optionally substituted with CH₃;
R² is F;
R³ is a 5- or 6-membered N-containing heteroaryl optionally substituted with CH₃, CD₃ or CHF₂, or a 9-membered N-containing bicyclic heterocycle optionally substituted with 1 or 2 substituents selected from oxo and CH₃;
R⁶ is H, CO₂R¹⁰, or

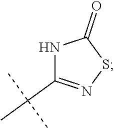

R⁷ is a 5- or 6-membered N-containing heteroaryl or phenyl, wherein the heteroaryl or phenyl is optionally substituted with R⁸ or with R⁸ and R⁹;
R⁸ is CF₃, halo or cyclopropyl;
R⁹ is:
   i) a 5- or 6-membered heterocycle optionally substituted with 1 to 3 substituents independently selected from:
      halo,
      OH,
      C₁-C₄alkoxy,
      a 4- to 6-membered heterocycle,
      CN,
      C₃-C₆cycloalkyl,
      CD₃,
      C₁-C₄haloalkyl,
      CR¹²R¹³OP(O)(OH)₂
      oxo, and
      C₁-C₄alkyl, wherein the C₁-C₄alkyl is optionally substituted with OH, N(CH₃)₂, or a 4- to 6-membered heterocycle,
   ii) a 9-membered N-containing bicyclic heterocycle,
   iii) C₁-C₄alkoxy,
   iv) C₃-C₅cycloalkyl,
   v) halo,
   vi) (CH₂)ₙC(O)R¹¹, wherein n is 0 or 1, R¹¹ is NR¹²R¹³ or a 4- to 6-membered heterocycle,
   vii) C₁-C₄alkyl, or
   viii) CN; and
R¹² and R¹³ are independently H or CH₃.

In an embodiment of Formula IV, there is a provided a compound of Formula IVa:

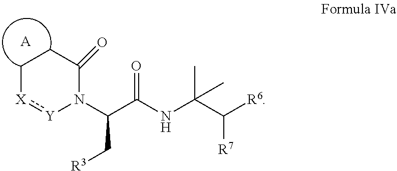

In an alternate embodiment, there is provided a compound of Formula I:

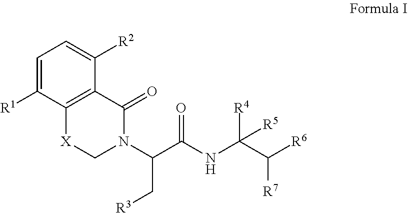

wherein
X is —CH₂—, —OCH₂— or —CH₂CH₂—;
R¹ is:
   i) a 8-, 9- or 10-membered N-containing bicyclic heterocycle optionally substituted with 1 to 3 substituents independently selected from:
      CN,
      halo,
      C₁-C₄alkyl optionally substituted with OCH₃,
      C₁-C₄haloalkyl,
      oxo,
      phenyl optionally substituted with 1 or 2 substituents independently selected from halo and OH, and
      benzyl optionally substituted with OCH₃,
   ii) phenyl or pyridyl optionally substituted with 1 or 2 substituents independently selected from:
      CN,
      NHC(O)pyridyl, wherein the pyridyl is optionally substituted with halo, and a 9-membered N-containing bicyclic heterocycle optionally substituted with CH₃, or
   iii) pyrazolyl optionally substituted with 1 to 3 substituents selected from CN and CH₃;
R² is H, halo or OCH₃;
R³ is a 5- or 6-membered N-containing heteroaryl optionally substituted with 1 or 2 substituents selected from: CH₃, CHF₂, OH and CH₂CN;
R⁴ and R⁵ are each CH₃ or together form a cyclopropyl;
R⁶ is H, OH, CH₃, CO₂R¹⁰, or

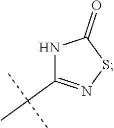

R⁷ is:
   i) phenyl optionally substituted with 1 or 2 substituents selected from:
      halo,
      CF₃, and
      pyrazine optionally substituted with CF₂H or OCH₃, or
   ii) a 5- or 6-membered N-containing heteroaryl optionally substituted with R⁸ or with R⁸ and R⁹;
R⁸ is CF₃, CF₂H, halo or cyclopropyl;
R⁹ is:
   i) a 5- or 6-membered heterocycle optionally substituted with 1 to 3 substituents independently selected from:
      halo,
      OH,
      OCH₃,
      SO₂NH₂,
      a 4- to 6-membered heterocycle,
      oxo, and
      C₁-C₄alkyl, wherein the C₁-C₄alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with CN, oxo, C(O)O(CH₃)₃ or OH,
   iii) C₁-C₄alkoxy,
   iv) C₃-C₅cycloalkyl,
   v) halo, or
   vi) C(O)OCH₃; and
R¹⁰ is H or C₁-C₄alkyl optionally substituted with OC(O)C₁-C₄alkyl or morpholine,
or a pharmaceutically acceptable salt thereof.

In an embodiment of Formula I, there is a provided a compound of Formula Ia:

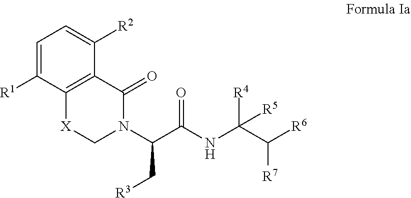

In an embodiment of Formula I, R² is F.

In an embodiment of Formula I, X is —CH₂—.

In an embodiment of Formula I, R⁴ and R⁵ are each CH₃.

In an embodiment of Formula I, R¹ is:
i) a 9-membered N-containing bicyclic heterocycle optionally substituted with 1 to 3 substituents independently selected from:
   CN,
   halo,
   C₁-C₄alkyl,
   oxo, and
   benzyl optionally substituted with OCH₃, or
ii) phenyl optionally substituted with 1 or 2 substituents independently selected from: CN, and
   a 9-membered N-containing bicyclic heterocycle optionally substituted with CH₃.

In an embodiment of Formula I, R³ is a 5-membered N-containing heteroaryl optionally substituted with CH₃ or CHF₂.

In an embodiment of Formula I, R⁶ is H, CO₂R¹⁰, or

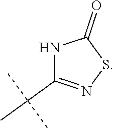

In an embodiment of Formula I, R⁷ is:
i) phenyl optionally substituted with halo or CF₃, or
ii) a 5- or 6-membered N-containing heteroaryl optionally substituted with R⁸ or with R⁸ and R⁹
wherein
R⁸ is CF₃, halo or cyclopropyl;
R⁹ is a 5- or 6-membered heterocycle optionally substituted with 1 to 3 substituents independently selected from:
   halo,
   OH,
   OCH₃,
   oxo, and
   C₁-C₄alkyl.

In an embodiment, there is a provided a compound of Formula II:

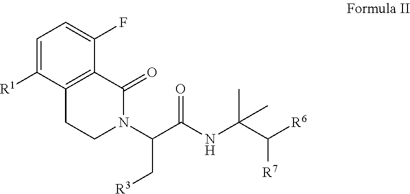

wherein
R¹ is:
   i) a 9-membered N-containing bicyclic heterocycle optionally substituted with 1 to 3 substituents independently selected from:
      CN,
      halo,
      C₁-C₄alkyl,
      oxo, and
      benzyl optionally substituted with OCH₃,
   ii) phenyl optionally substituted with 1 or 2 substituents independently selected from: CN, and
      a 9-membered N-containing bicyclic heterocycle optionally substituted with CH₃;
R³ is a 5-membered N-containing heteroaryl optionally substituted with CH₃ or CHF₂;
R⁶ is H, CO₂R¹⁰, or

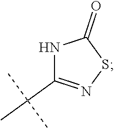

R⁷ is:
   i) phenyl optionally substituted with halo or CF₃, or
   ii) a 5- or 6-membered N-containing heteroaryl optionally substituted with R⁸ or with R⁸ and R⁹;
R⁸ is CF₃, halo or cyclopropyl;
R⁹ is a 5- or 6-membered heterocycle optionally substituted with 1 to 3 substituents independently selected from:
   halo,
   OH,
   OCH₃,
   oxo, and
   C₁-C₄alkyl, and
R¹⁰ is H or C₁-C₄alkyl optionally substituted with OC(O)C₁-C₄alkyl or morpholine,
or a pharmaceutically acceptable salt thereof.

In an embodiment of Formula II, there is a provided a compound of Formula IIa:

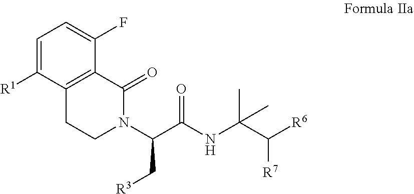

The embodiments below are embodiments of any or all of the applicable formulae above.

In an embodiment, A is

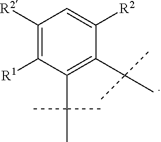

In a particular embodiment, R² is F and R^{2′} is H.

In an embodiment, X is CH₂ or CH₂CH₂, Y is CH₂ and [symbol] is a single bond. In a particular embodiment, X is CH₂, Y is CH₂ and [symbol] is a single bond.

In an embodiment, R¹ is:

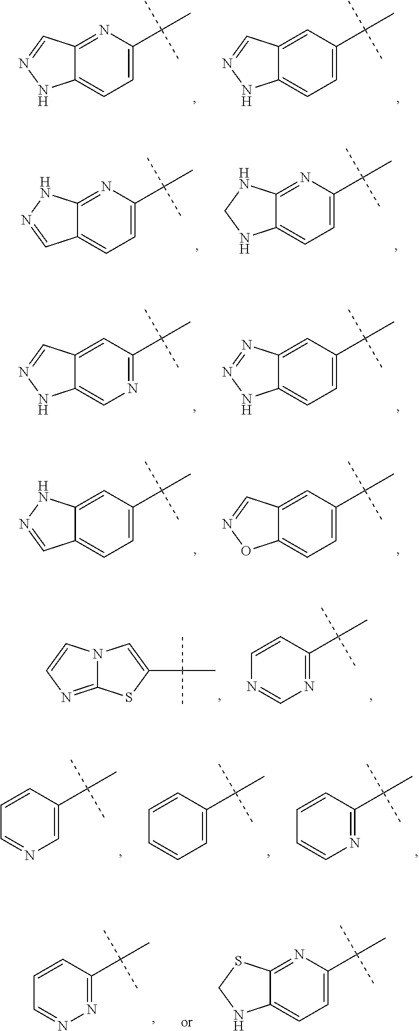

optionally substituted with 1 to 3 substituents independently selected from:
CN,
halo,
C₁-C₄alkyl,
C₁-C₄haloalkyl,
C₁-C₄alkoxy,
C₁-C₄haloalkoxy,
CD₃,
oxo,
benzyl optionally substituted with OCH₃, and
a 9-membered N-containing bicyclic heterocycle optionally substituted with CH₃.

In an embodiment, R¹ is:

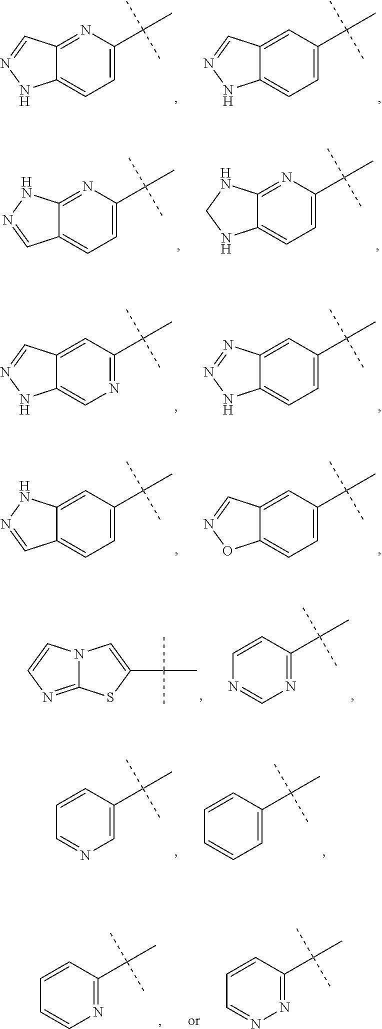

optionally substituted with 1 to 3 substituents independently selected from:
CN,
halo,
C₁-C₄alkyl,
C₁-C₄haloalkyl,
C₁-C₄alkoxy,
C₁-C₄haloalkoxy,
CD₃,
oxo,
benzyl optionally substituted with OCH₃, and
a 9-membered N-containing bicyclic heterocycle optionally substituted with CH₃.

In an embodiment, R¹ is:

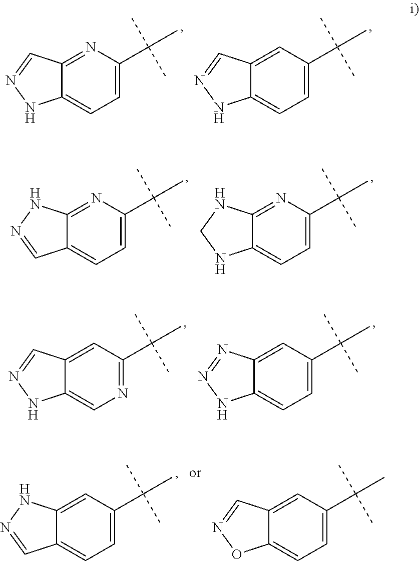

optionally substituted with 1 to 3 substituents independently selected from:
CN,
halo,
C₁-C₄alkyl,
oxo, and
benzyl optionally substituted with OCH₃, or
ii) phenyl optionally substituted with 1 or 2 substituents independently selected from: CN, and
a 9-membered N-containing bicyclic heterocycle optionally substituted with CH₃.

In an embodiment, R¹ is:

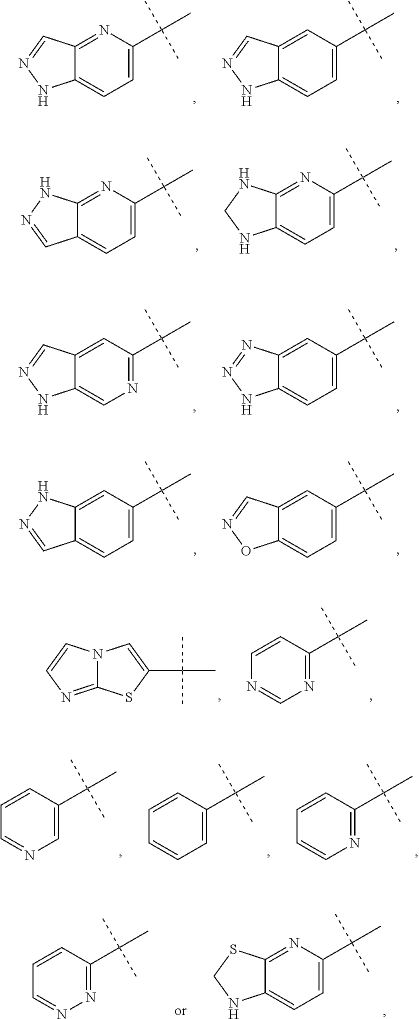

optionally substituted with 1 to 3 substituents independently selected from:
CN,
F,
Cl,
CH₃,
CF₃,
OCH₃,
OCHF₂,
CD₃,
oxo, and
benzyl substituted with OCH₃, or
a 9-membered N-containing bicyclic heterocycle substituted with CH₃.

In an embodiment, R¹ is:

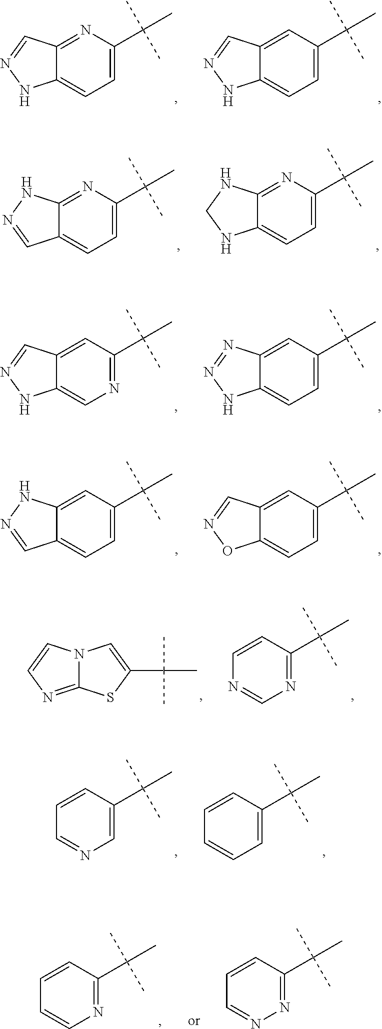

optionally substituted with 1 to 3 substituents independently selected from:
CN,
F,
Cl,
CH₃,
CF₃,
OCH₃,
OCHF₂,
CD₃,
oxo, and
benzyl substituted with OCH₃, or
a 9-membered N-containing bicyclic heterocycle substituted with CH₃.

In an embodiment, R¹ is:

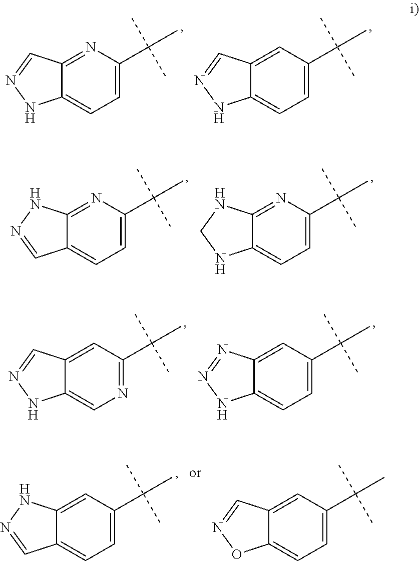

optionally substituted with 1 to 3 substituents independently selected from:
   CN,
   F,
   CH₃,
   oxo, and
   benzyl substituted with OCH₃, or
ii) phenyl substituted with CN and a 9-membered N-containing bicyclic heterocycle substituted with CH₃.

In an embodiment, R¹ is:

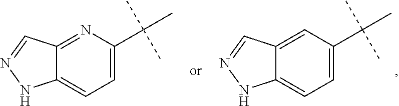

optionally substituted with 1 to 3 substituents independently selected from:
   CN,
   F, and
   CH₃.

In a further embodiment, R¹ is selected from:

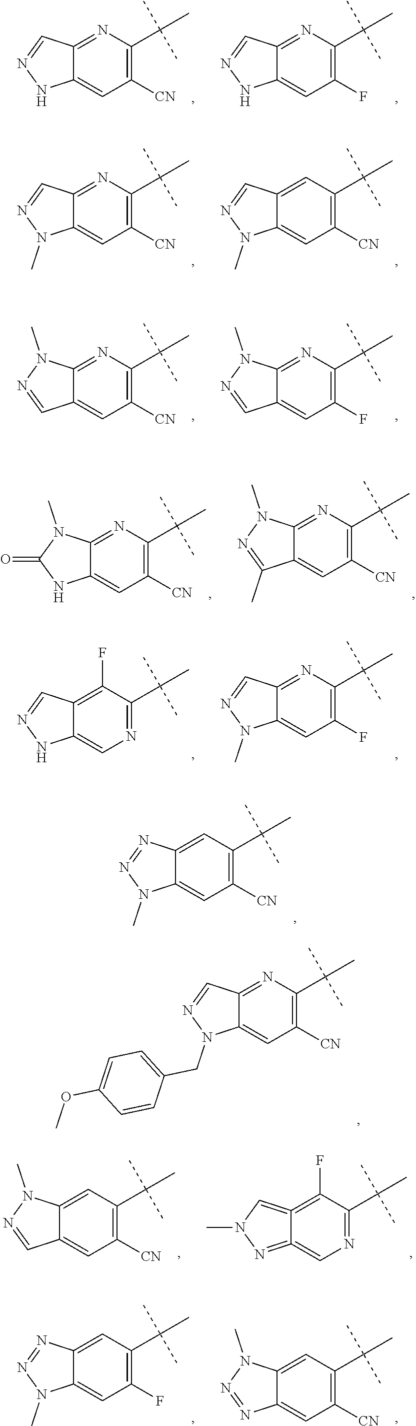

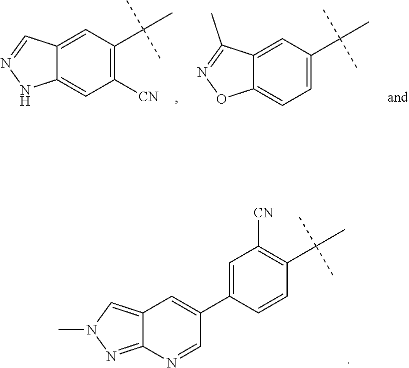

In a further embodiment, R¹ is selected from:

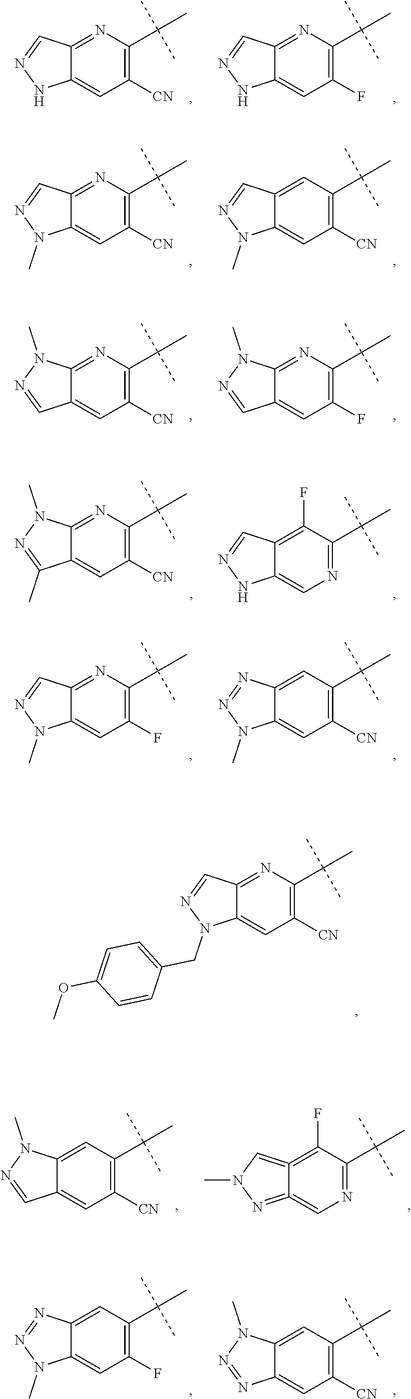

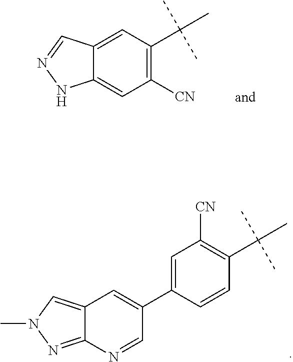

In a further embodiment, R³ is pyrazole substituted with CH₃, CHF₂ or CD₃, isothiazole, imidazopyrimidine, imidazopyrazinone substituted with CH₃, or pyridine substituted with CH₃. In a particular embodiment, R³ is:

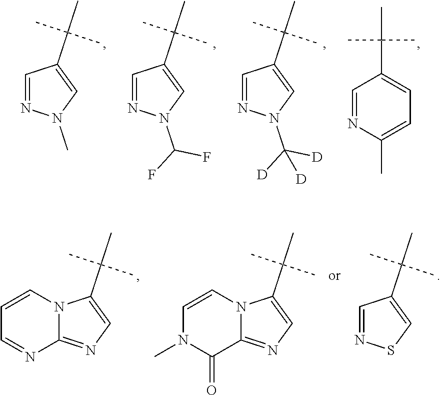

In a further embodiment, R³ is pyrazole substituted with CH₃ or CHF₂. In a particular embodiment, R³ is:

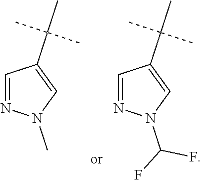

In a particular embodiment, R³ is:

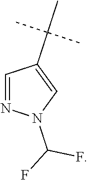

In an embodiment, R⁶ is H, OH, CO₂R¹⁰,

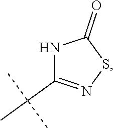

or R¹⁰ is H, CH₂CH₃,

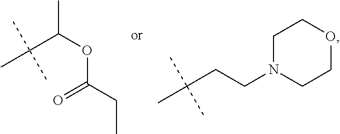

and R^{6′} is H; or R⁶ is D and R^{6′} is D. In an embodiment, R⁶ is H, CO₂R¹⁰, or

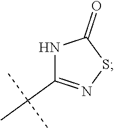

and R¹⁰ is H, CH₂CH₃,

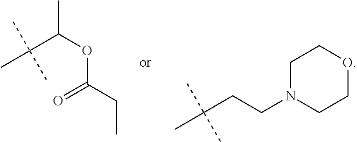

In a particular embodiment, R⁶ is H and R^{6′} is H.

In an alternate embodiment, R⁶ and R⁷ together form:

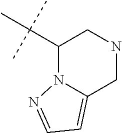

optionally substituted with 1 to 3 substituents independently selected from: CH₃, CF₃ and oxo, and R^{6′} is H.

In a further embodiment, R⁷ is:

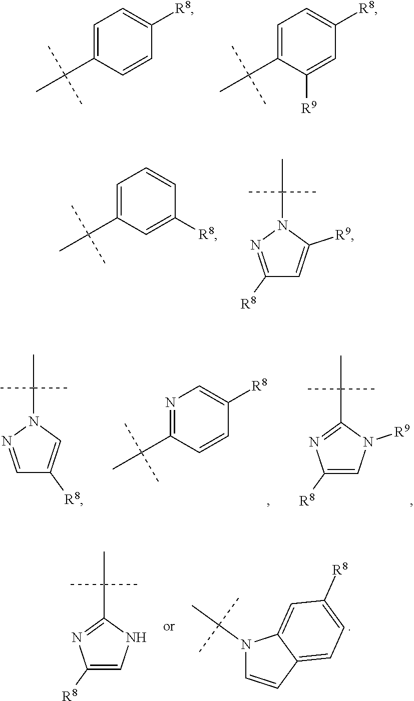

In a further embodiment, R⁷ is:

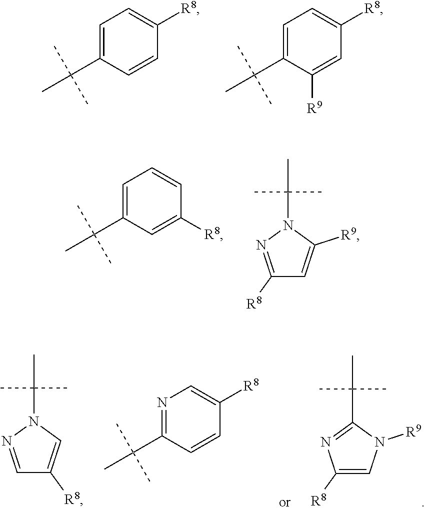

In a further embodiment, R⁷ is:

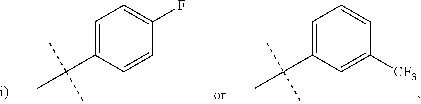

or
ii) a 5- or 6-membered N-containing heteroaryl selected from:

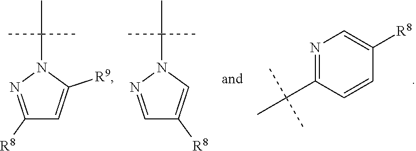

In a further embodiment, R⁷ is:

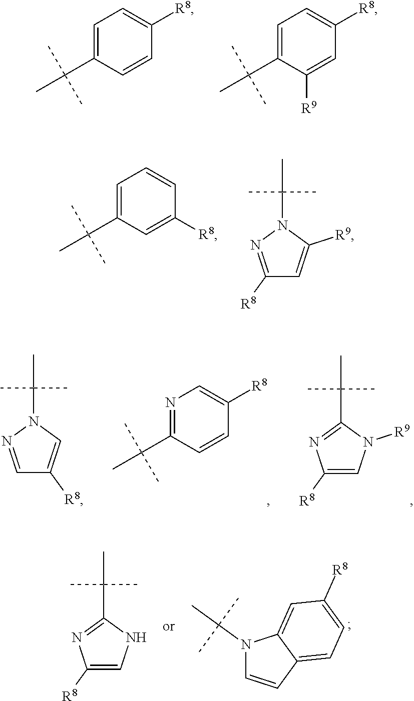

wherein Ra is CF₃, F, Cl, cyclopropyl, CHs or H, and R′ is
   i) a 5- or 6-membered heterocycle selected from:

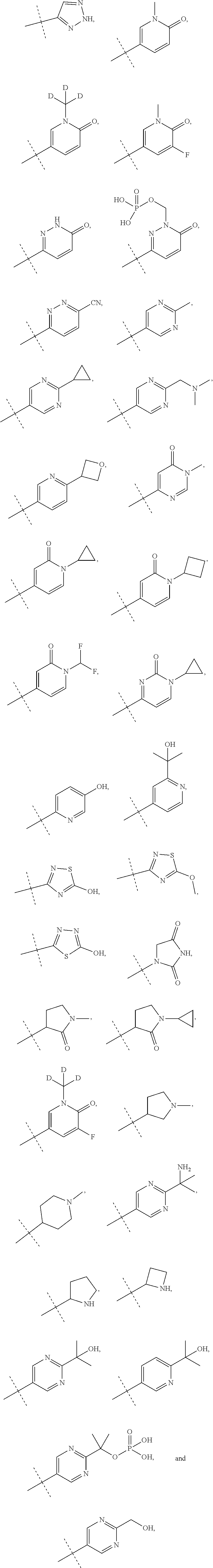

ii) a 9-membered N-containing bicyclic heterocycle which is

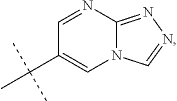

iii) OCH₃,
iv) cyclopropyl,
v) Br,
vi) C(O)N(CH₃)₂, CH₂C(O)NH(CH₃),

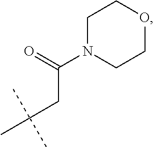

vii) CH₃,

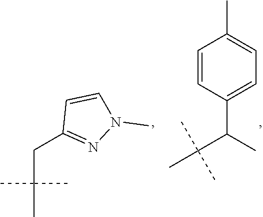

CH₂NHS(O)₂CH₃, CH₂NH₂,
viii) CN,
ix) or

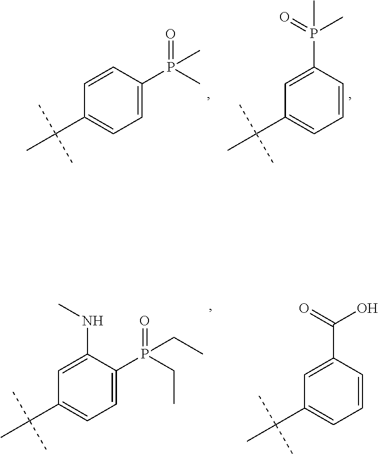

x) P(O)(CH₃)₂.

In a further embodiment, R⁷ is:

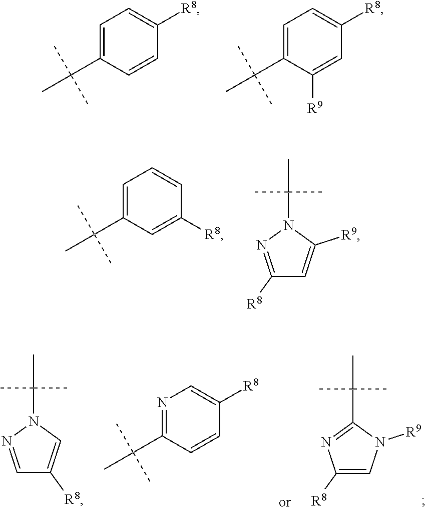

wherein R⁸ is CF₃, F or cyclopropyl, and R⁹ is
   i) a 5- or 6-membered heterocycle selected from:

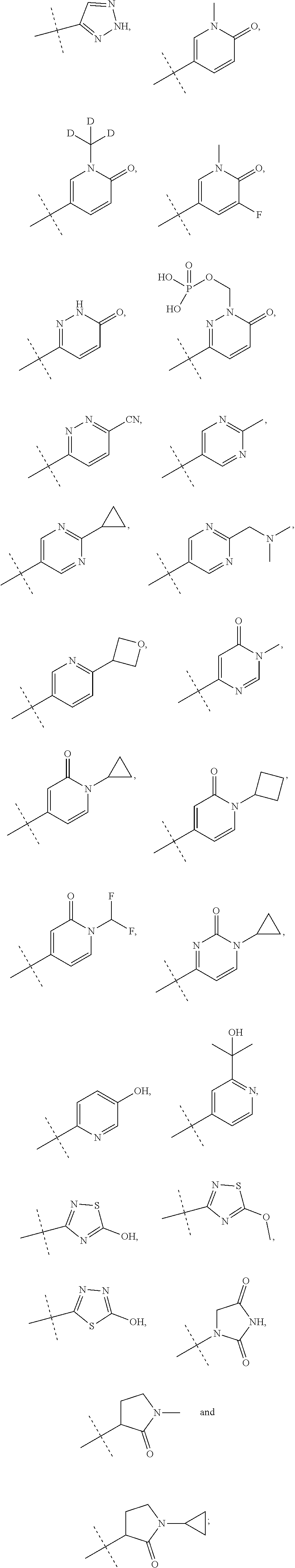

ii) a 9-membered N-containing bicyclic heterocycle which is

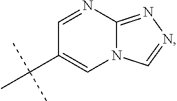

iii) OCH₃,
iv) cyclopropyl,
v) Br,
vi) C(O)N(CH₃)₂, CH₂C(O)NH(CH₃),

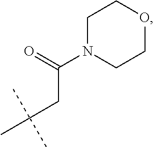

vii) CH₃,

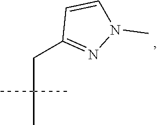

or
viii) CN.

In a further embodiment, R⁷ is:
i)

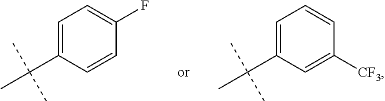

or
ii) a 5- or 6-membered N-containing heteroaryl selected from:

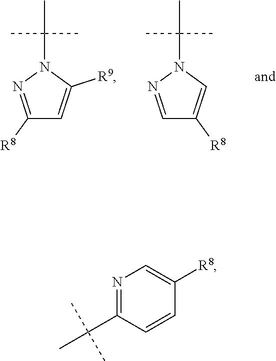

wherein R⁸ is CF₃, F or cyclopropyl, and R′ is a 5- or 6-membered heterocycle selected from:

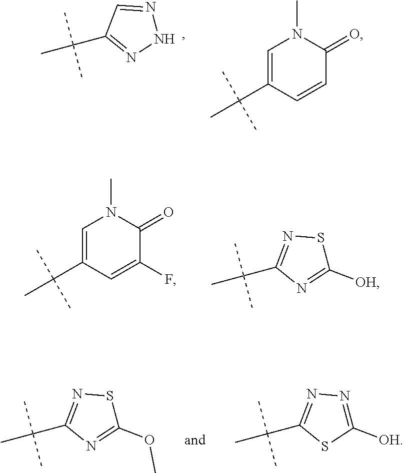

In a further embodiment, R⁷ is:

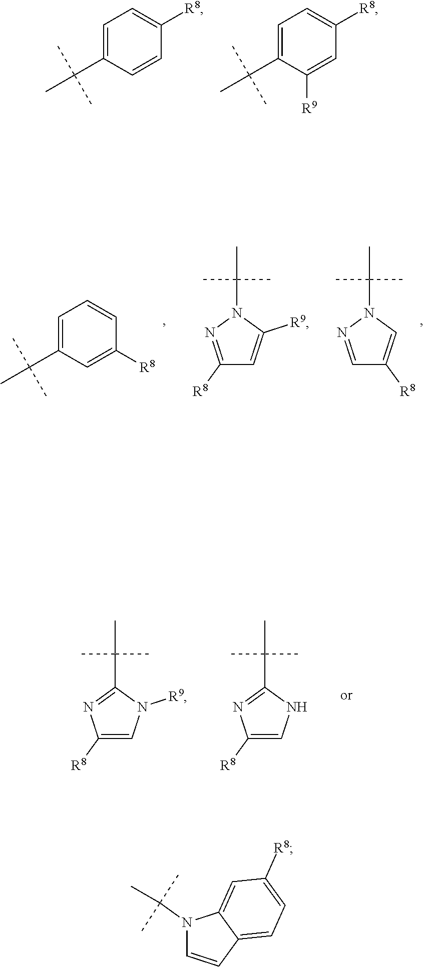

wherein R⁸ is CF₃, F, Cl, cyclopropyl, CH₃ or H, and R⁹ is
   i) a 5- or 6-membered heterocycle selected from:

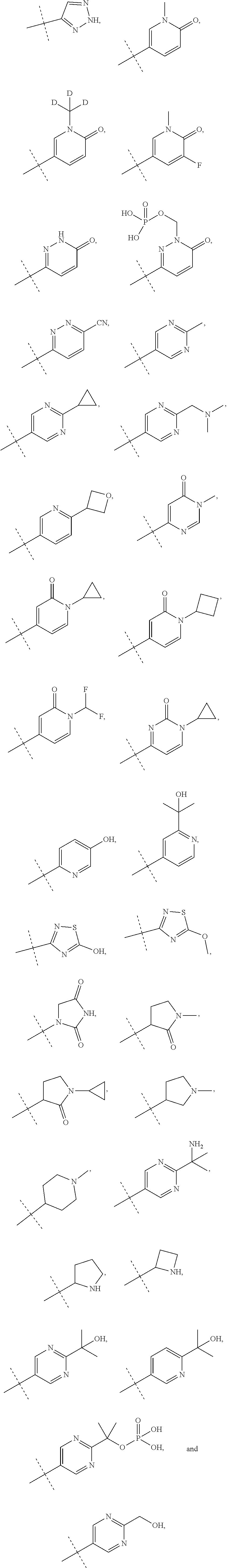

ii) a 9-membered N-containing bicyclic heterocycle which is

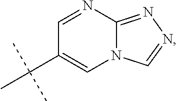

iii) OCH₃,
iv) cyclopropyl,
v) Br,
vi) C(O)N(CH₃)₂, CH₃C(O)NH(CH₃),

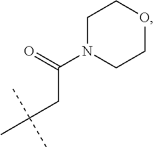

vii) CH₃,

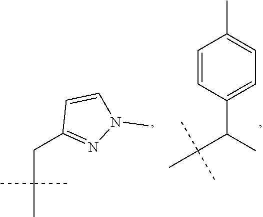

CH₂NHS(O)₂CH₃, CH₂NH₂,
viii) CN, or
ix)

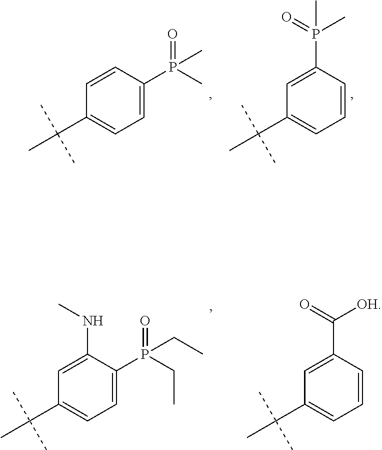

In a further embodiment, R⁷ is:

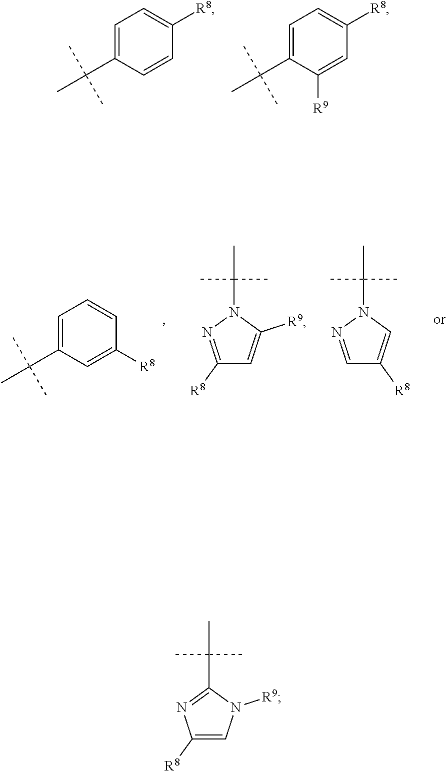

wherein R⁸ is CF₃, F or cyclopropyl, and R⁹ is
i) a 5- or 6-membered heterocycle selected from:

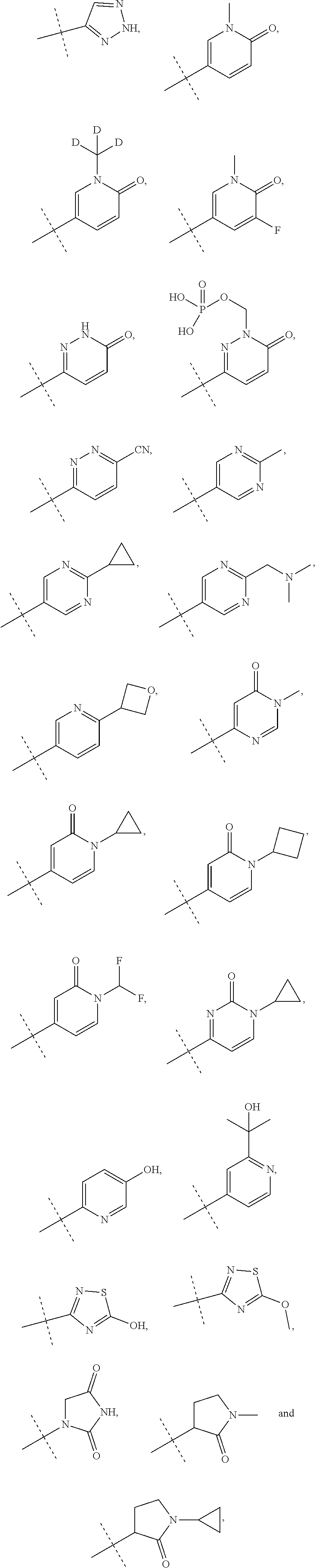

ii) a 9-membered N-containing bicyclic heterocycle which is

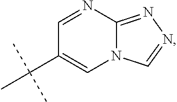

iii) OCH₃,
iv) cyclopropyl,
v) Br,

vi) C(O)N(CH₃)₂, CH₃C(O)NH(CH₃),

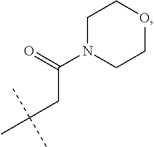

vii) CH₃,

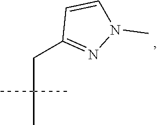

or
viii) CN.

In a further embodiment, R⁷ is:

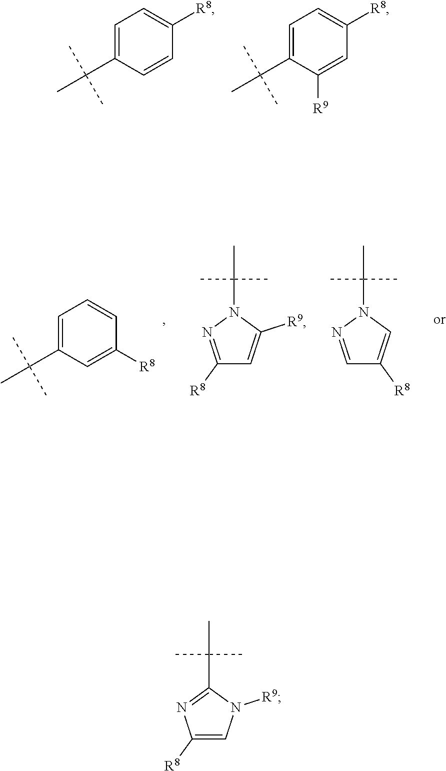

wherein R⁸ is CF₃, F or cyclopropyl, and R⁹ is
   i) a 5- or 6-membered heterocycle selected from:

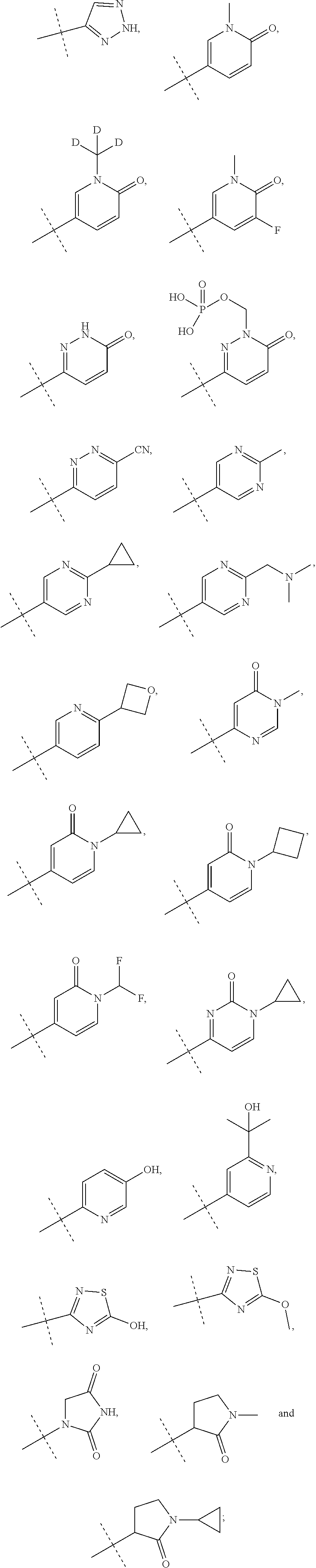

ii) a 9-membered N-containing bicyclic heterocycle which is

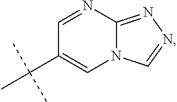

iii) OCH₃,
iv) cyclopropyl,
v) Br,
vi) C(O)N(CH₃)₂, CH₃C(O)NH(CH₃),

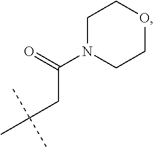

vii) CH₃,

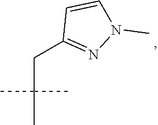

or
viii) CN.

In a further embodiment, R⁷ is:
i)

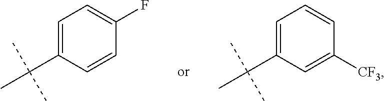

or
ii) a 5- or 6-membered N-containing heteroaryl selected from:

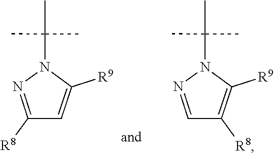

wherein R⁸ is CF₃, F or cyclopropyl, and R⁹ is a 5- or 6-membered heterocycle selected from:

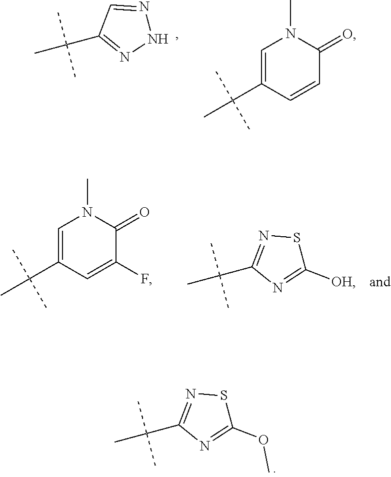

In a particular embodiment, R⁷ is

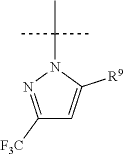

and R⁹ is selected from:

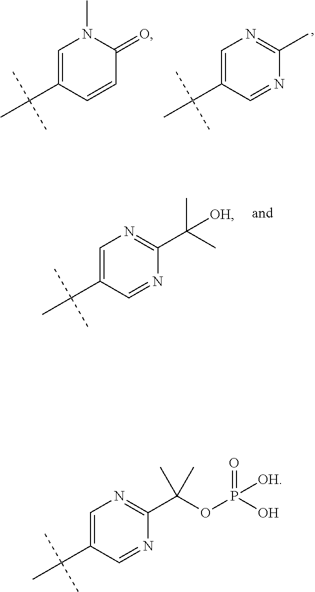

In a further embodiment, R⁷ is selected from:

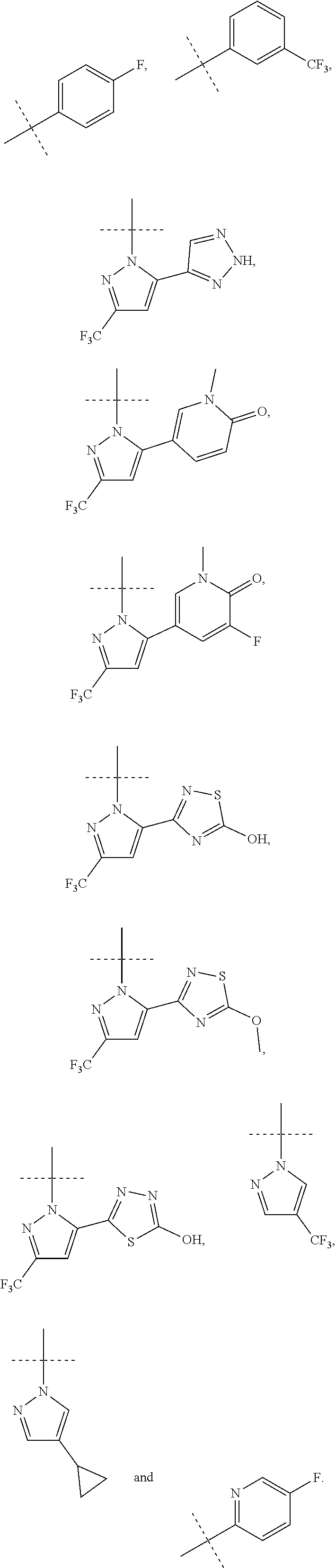

In a further embodiment, R⁷ is selected from:

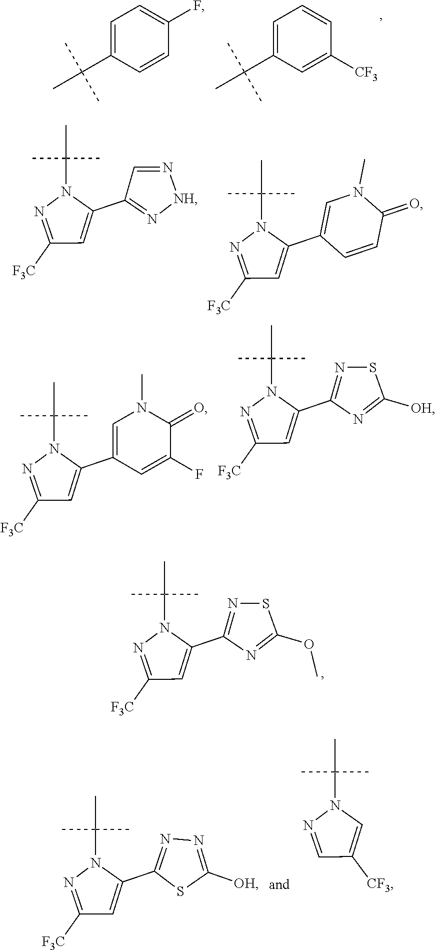

In one embodiment, the compound is selected from:

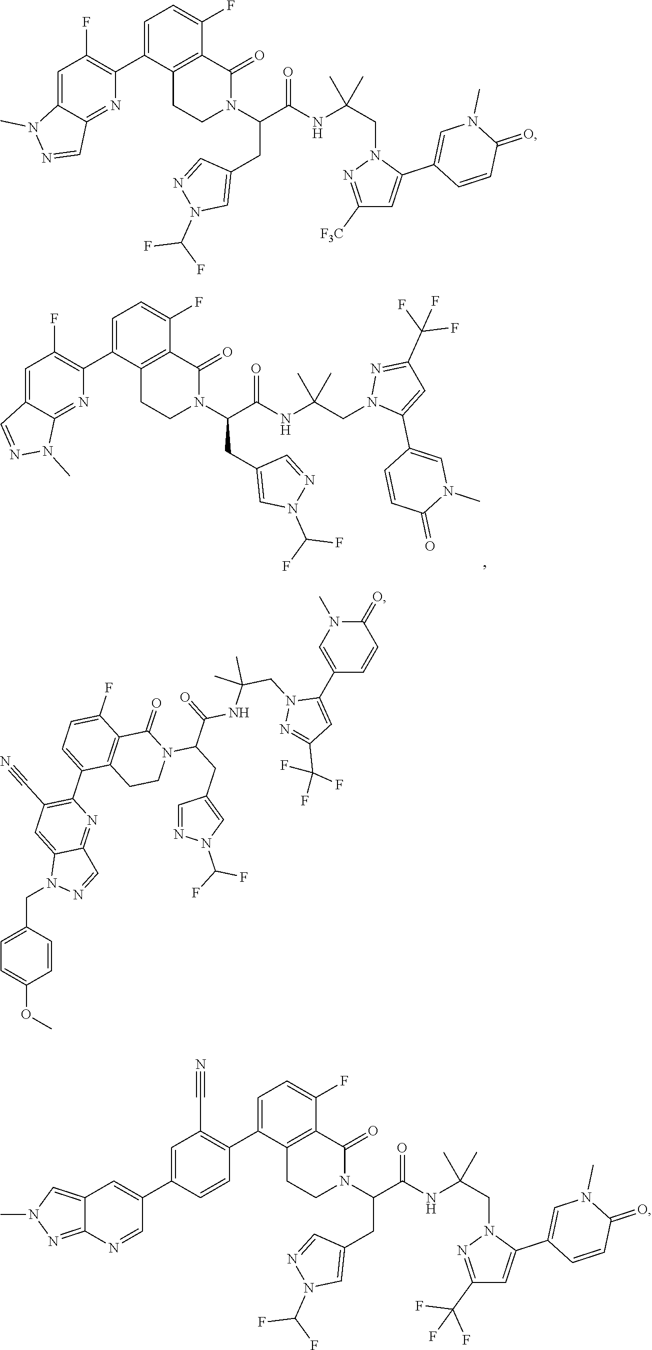

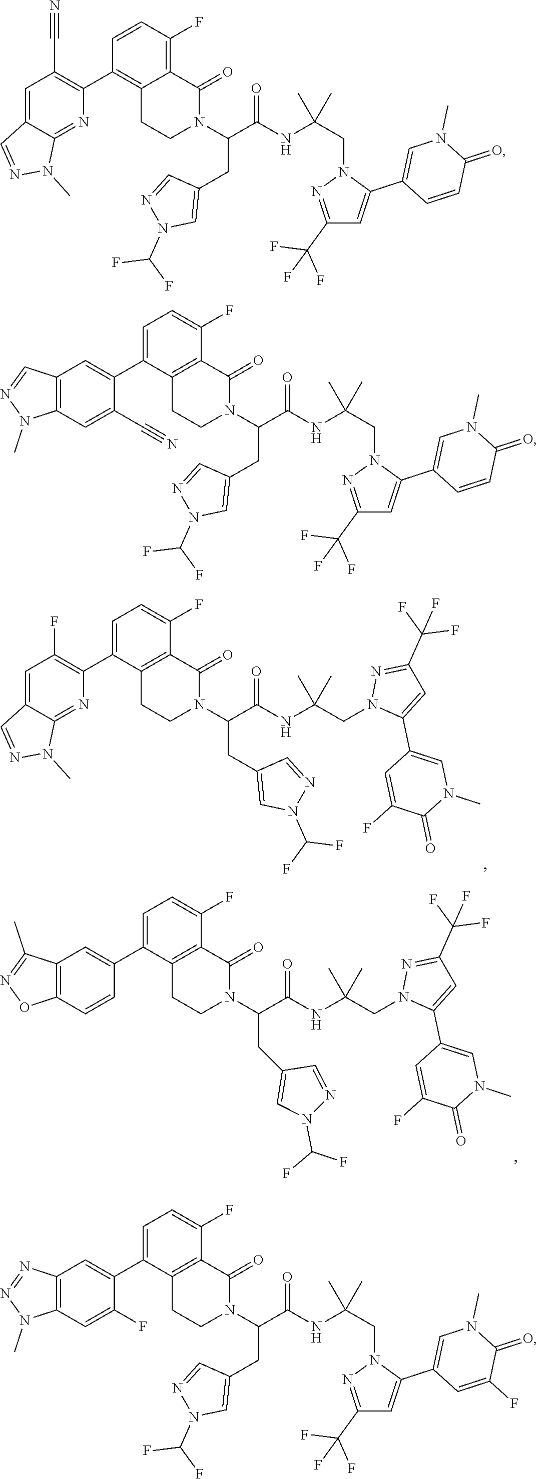

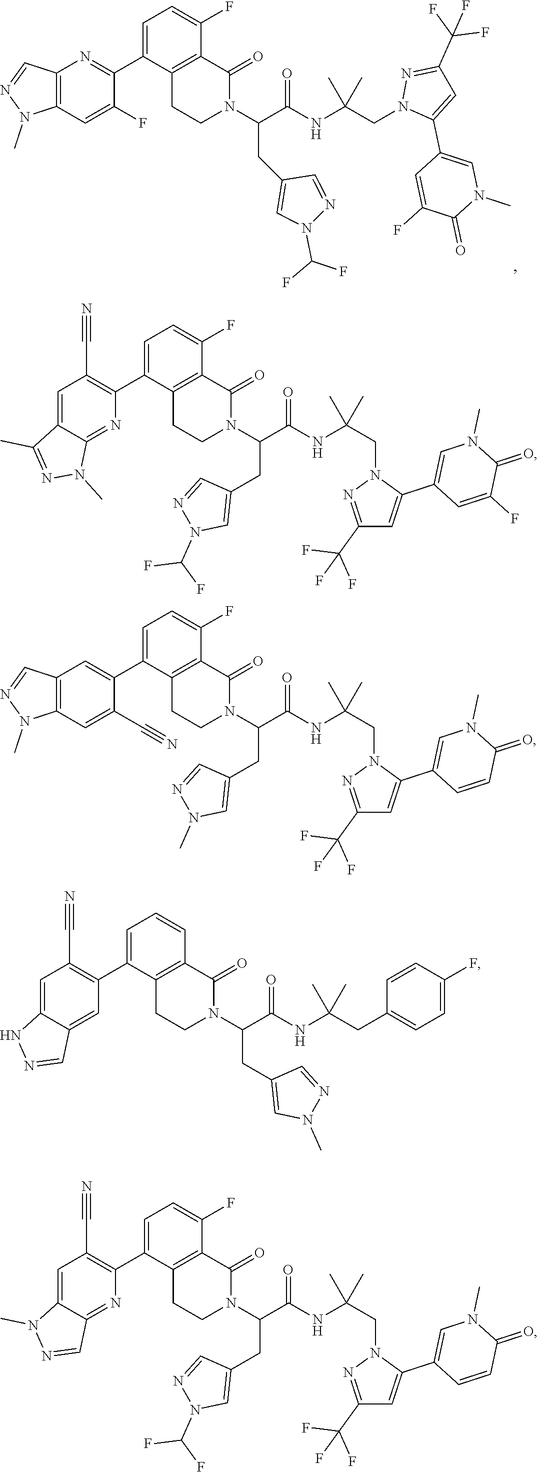

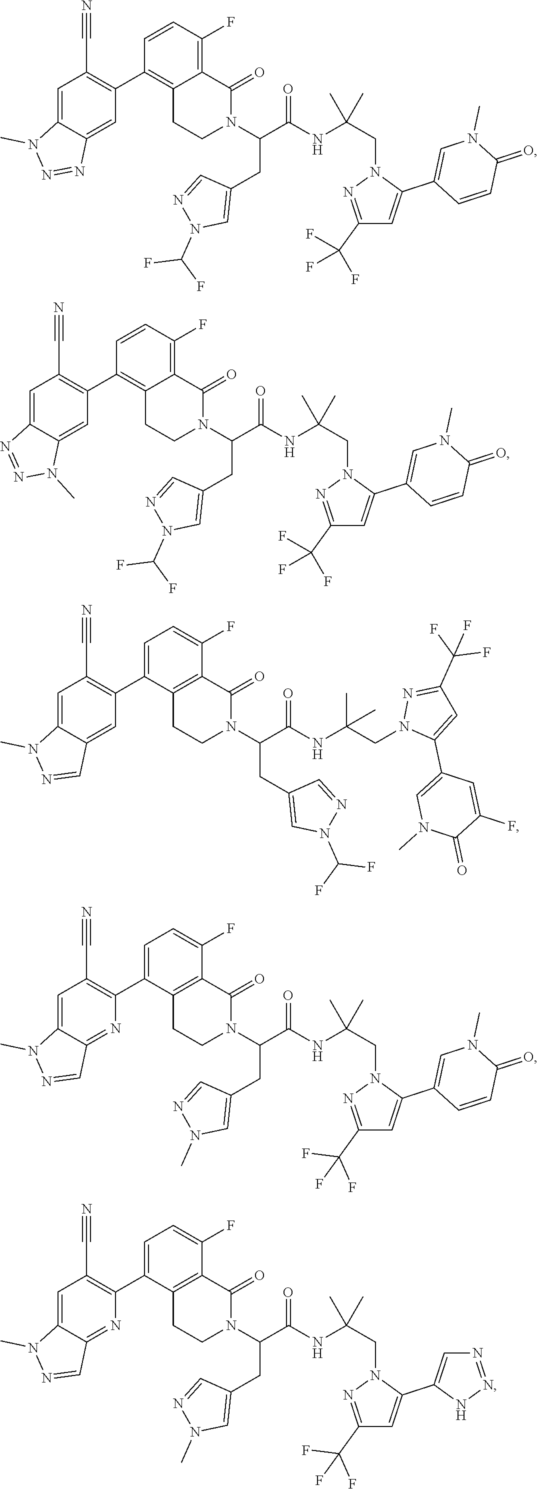

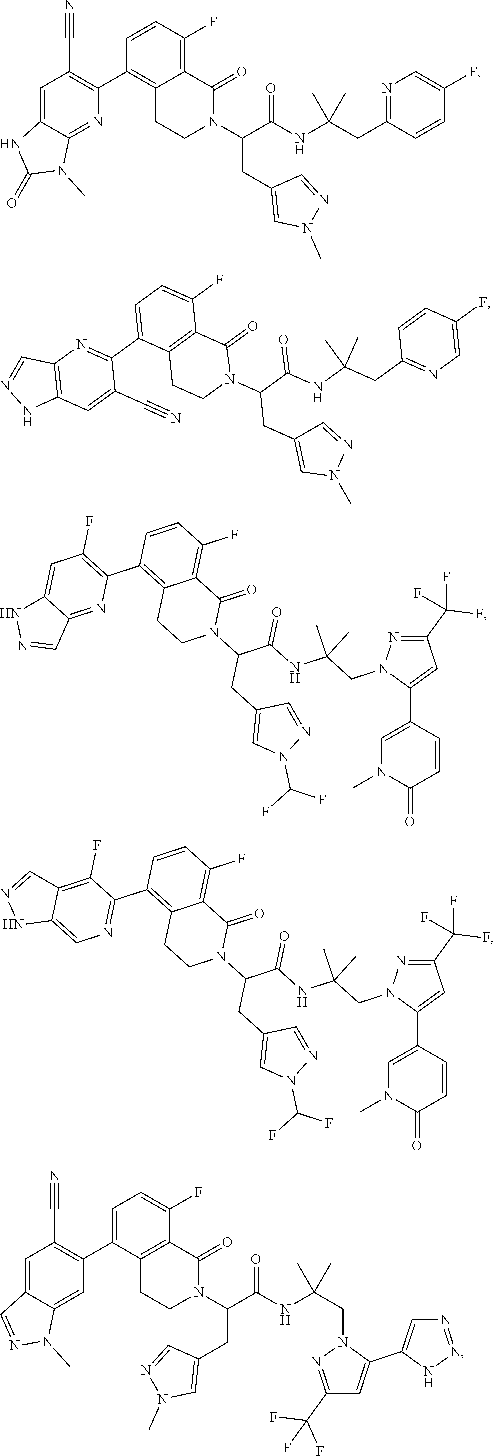 [SMILES: Cn1cc(CC(C(=O)NC(C)(C)Cc2ccc(F)cn2)N2CCc3c(-c4nc5c(cc4C#N)[nH]c(=O)n5C)ccc(F)c3C2=O)cn1 ; Cn1cc(CC(C(=O)NC(C)(C)Cc2ccc(F)cn2)N2CCc3c(-c4nc5cn[nH]c5cc4C#N)ccc(F)c3C2=O)cn1 ; Cn1cc(-c2cc(C(F)(F)F)nn2CC(C)(C)NC(=O)C(Cc2cnn(C(F)F)c2)N2CCc3c(-c4nc5cn[nH]c5cc4F)ccc(F)c3C2=O)ccc1=O ; Cn1cc(-c2cc(C(F)(F)F)nn2CC(C)(C)NC(=O)C(Cc2cnn(C(F)F)c2)N2CCc3c(-c4ncc5[nH]ncc5c4F)ccc(F)c3C2=O)ccc1=O ; Cn1cc(CC(C(=O)NC(C)(C)Cn2nc(C(F)(F)F)cc2-c2cnn[nH]2)N2CCc3c(-c4cc5c(cnn5C)cc4C#N)ccc(F)c3C2=O)cn1]

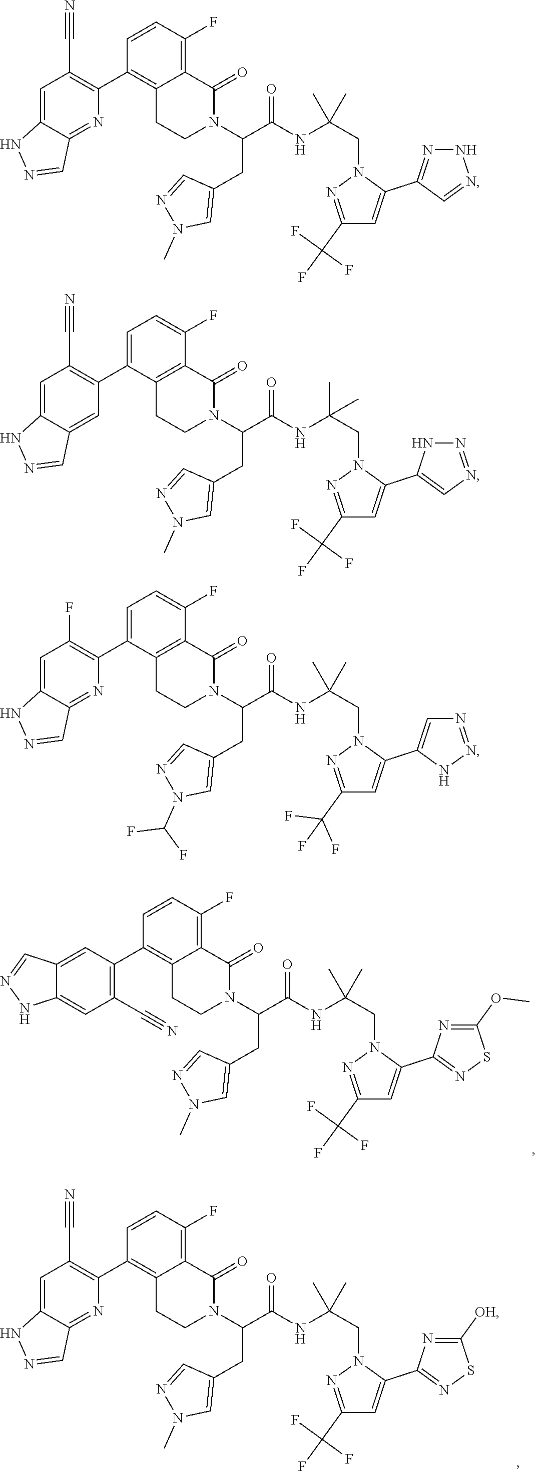

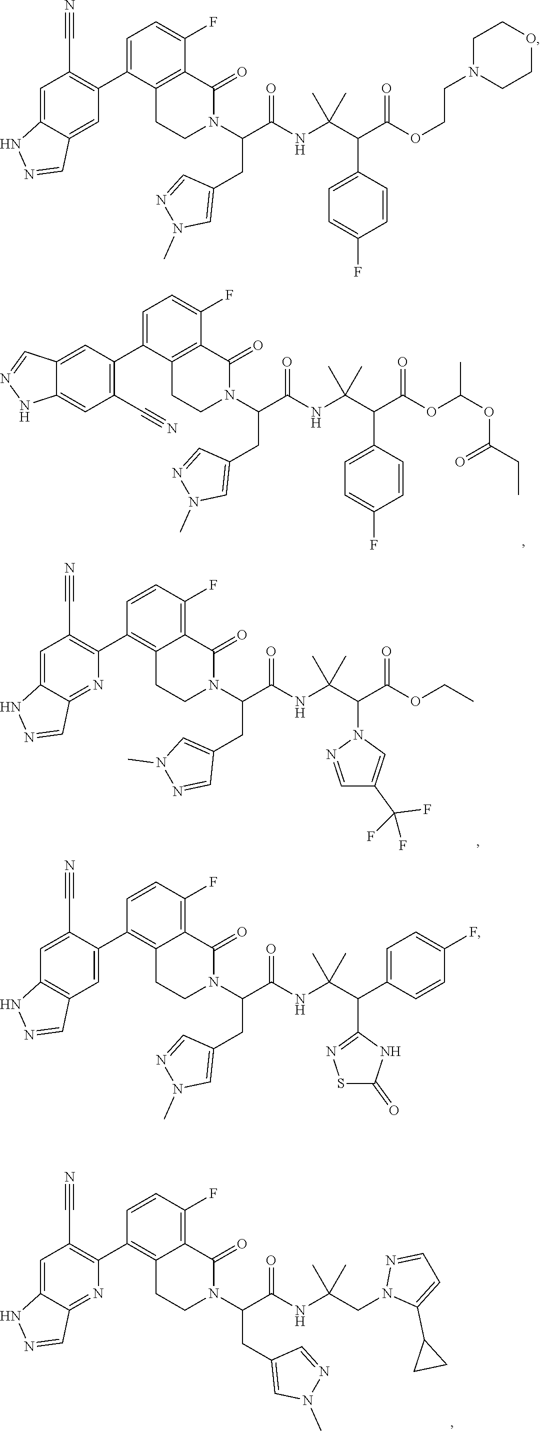

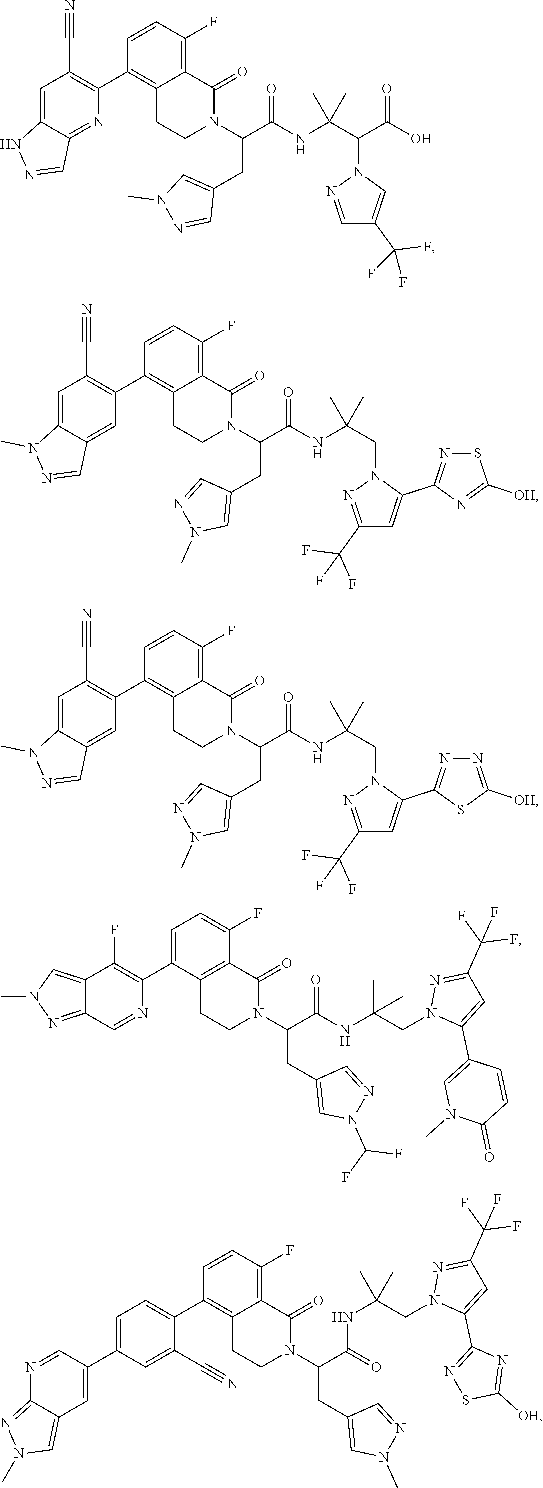

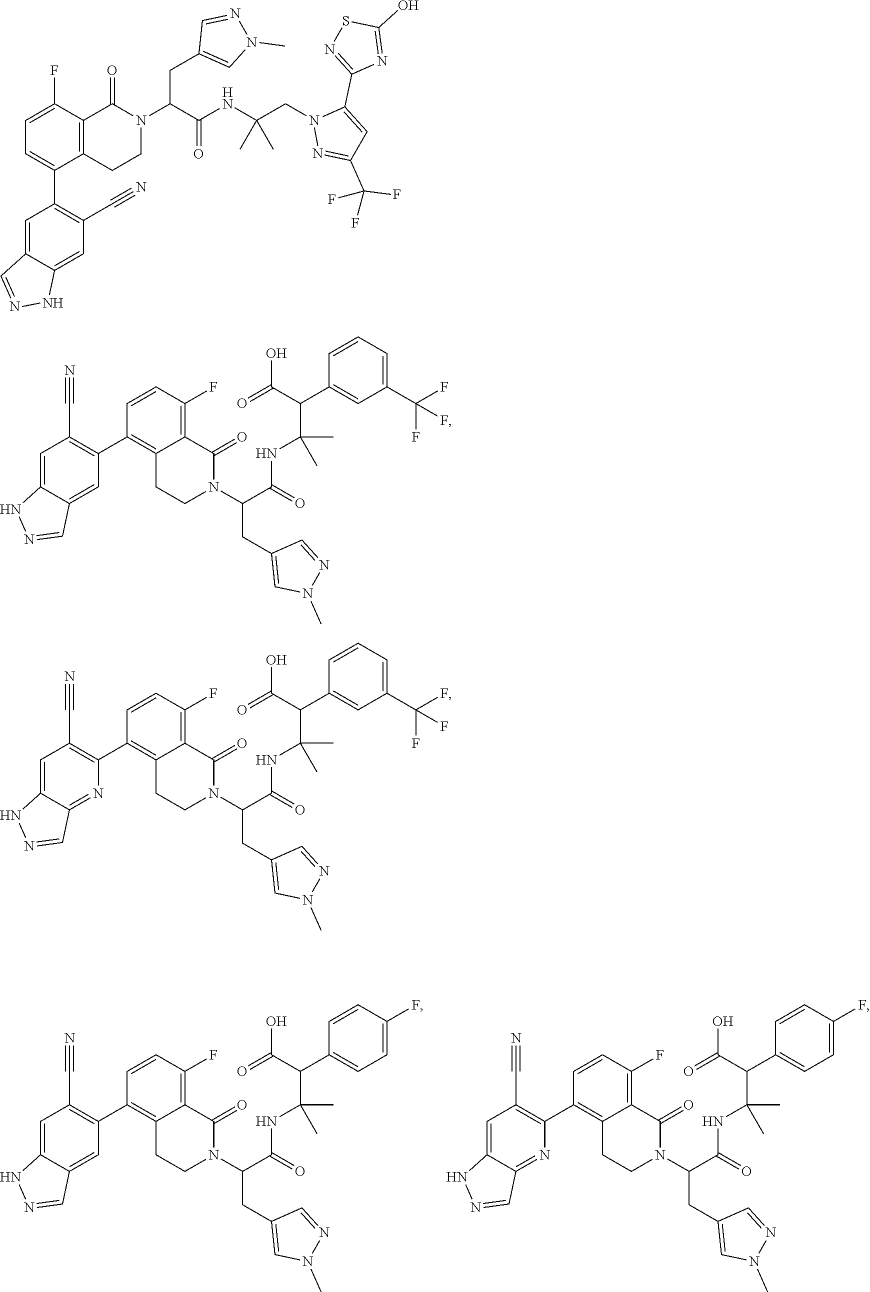

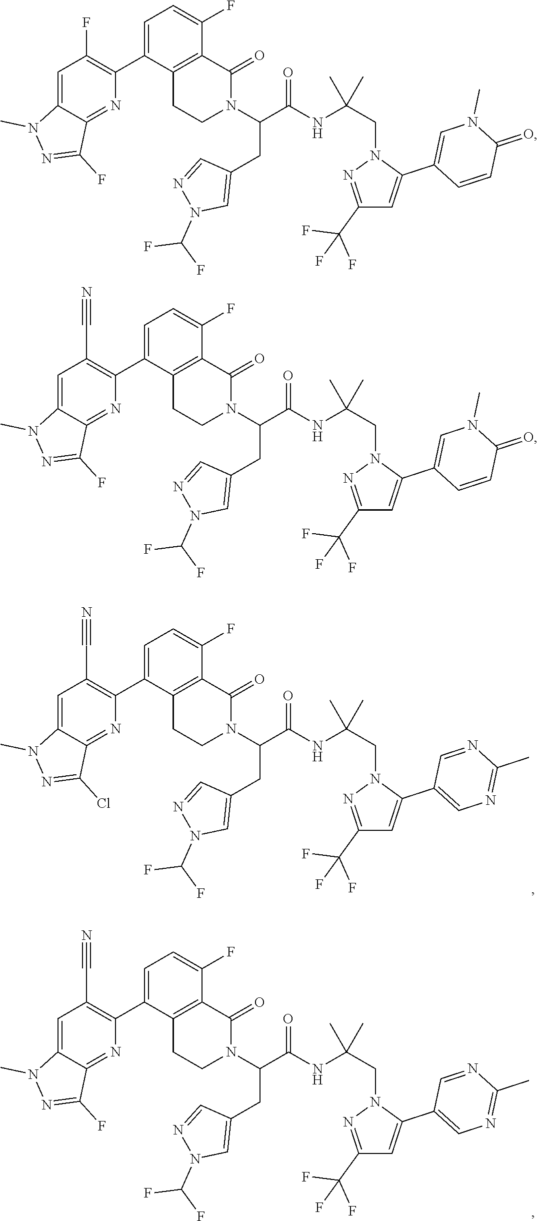

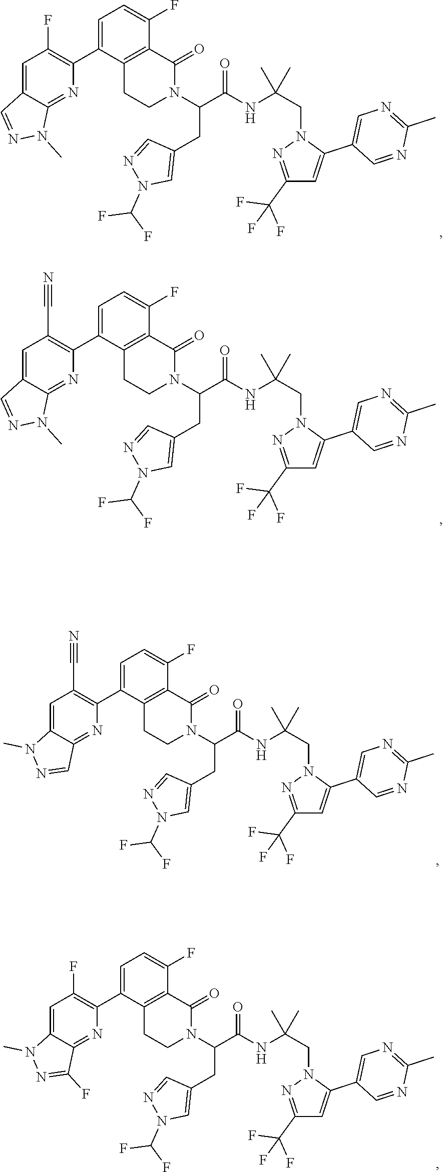

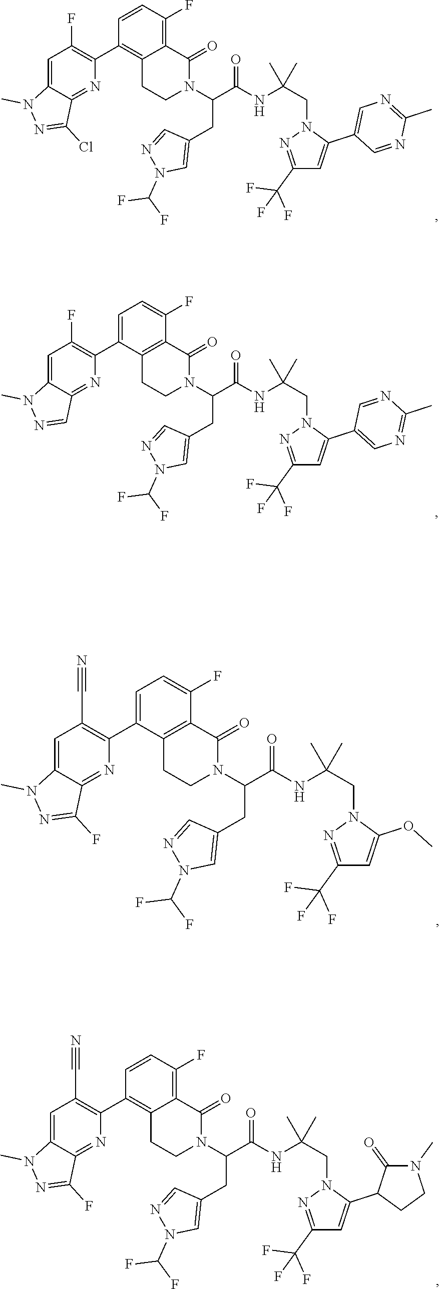

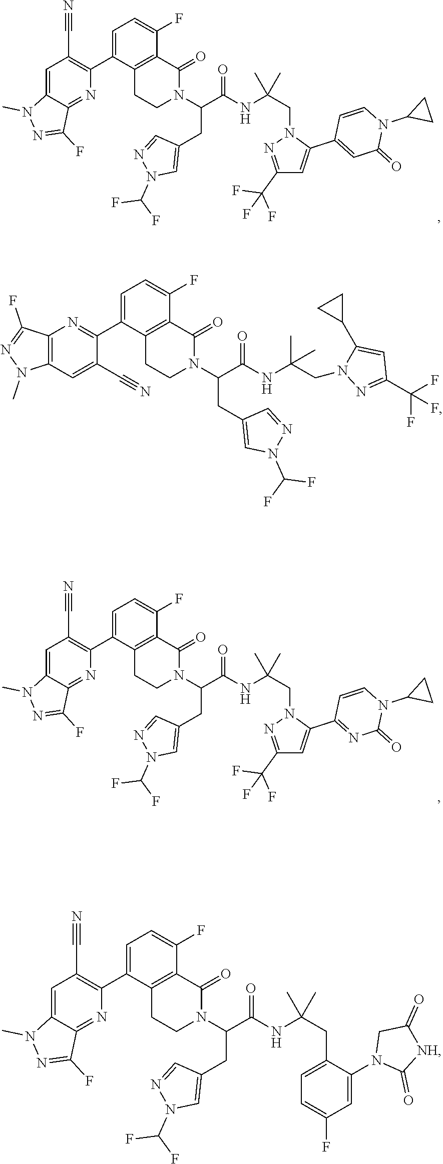 [SMILES: Cn1nc(F)c2nc(-c3ccc(F)c4c3CCN(C(Cc3cnn(C(F)F)c3)C(=O)NC(C)(C)Cn3nc(C(F)(F)F)cc3-c3ccn(C5CC5)c(=O)c3)C4=O)c(C#N)cc21 ; Cn1nc(F)c2nc(-c3ccc(F)c4c3CCN(C(Cc3cnn(C(F)F)c3)C(=O)NC(C)(C)Cn3nc(C(F)(F)F)cc3C3CC3)C4=O)c(C#N)cc21 ; Cn1nc(F)c2nc(-c3ccc(F)c4c3CCN(C(Cc3cnn(C(F)F)c3)C(=O)NC(C)(C)Cn3nc(C(F)(F)F)cc3-c3ccn(C5CC5)c(=O)n3)C4=O)c(C#N)cc21 ; Cn1nc(F)c2nc(-c3ccc(F)c4c3CCN(C(Cc3cnn(C(F)F)c3)C(=O)NC(C)(C)Cc3ccc(F)cc3N3CC(=O)NC3=O)C4=O)c(C#N)cc21]

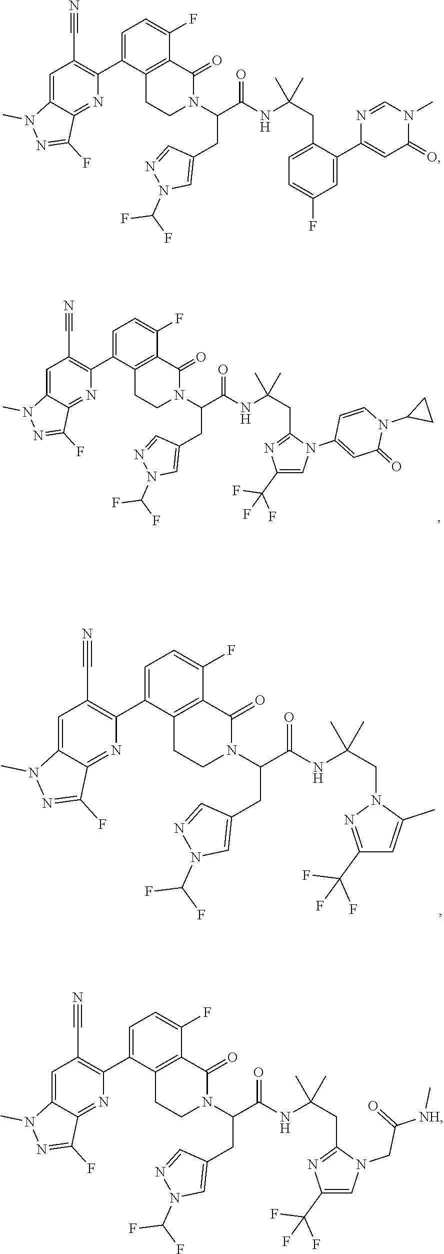

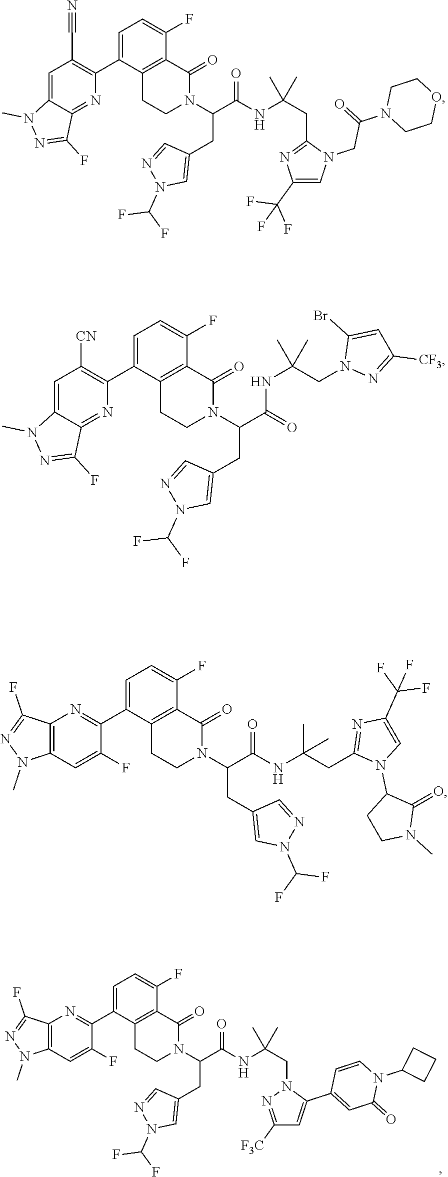 [SMILES: Cn1nc(F)c2nc(-c3ccc(F)c4c3CCN(C(Cc3cnn(C(F)F)c3)C(=O)NC(C)(C)Cc3nc(C(F)(F)F)cn3CC(=O)N3CCOCC3)C4=O)c(C#N)cc21 ; Cn1nc(F)c2nc(-c3ccc(F)c4c3CCN(C(Cc3cnn(C(F)F)c3)C(=O)NC(C)(C)Cn3nc(C(F)(F)F)cc3Br)C4=O)c(C#N)cc21 ; CN1CCC(n2cc(C(F)(F)F)nc2CC(C)(C)NC(=O)C(Cc2cnn(C(F)F)c2)N2CCc3c(-c4nc5c(F)nn(C)c5cc4F)ccc(F)c3C2=O)C1=O ; Cn1nc(F)c2nc(-c3ccc(F)c4c3CCN(C(Cc3cnn(C(F)F)c3)C(=O)NC(C)(C)Cn3nc(C(F)(F)F)cc3-c3ccn(C5CCC5)c(=O)c3)C4=O)c(F)cc21]

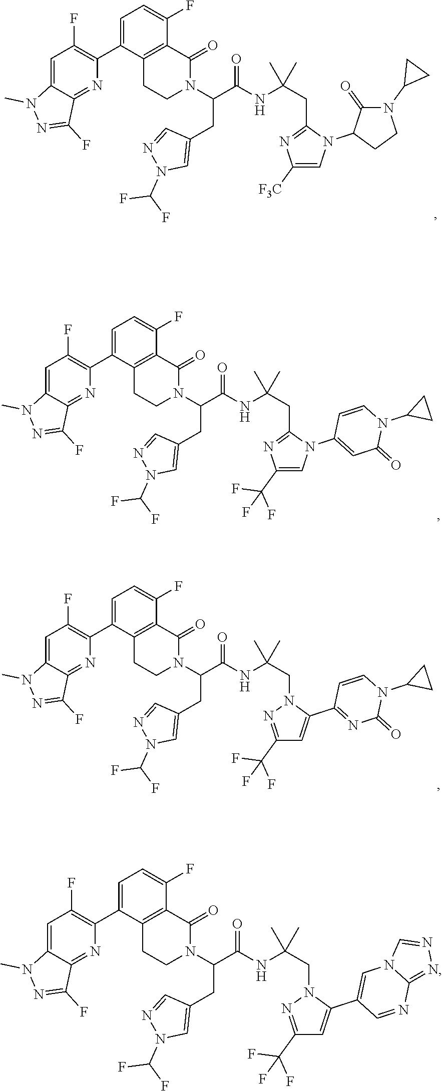 [SMILES: Cn1nc(F)c2nc(-c3ccc(F)c4c3CCN(C(Cc3cnn(C(F)F)c3)C(=O)NC(C)(C)Cc3nc(C(F)(F)F)cn3C3CCN(C5CC5)C3=O)C4=O)c(F)cc21 ; Cn1nc(F)c2nc(-c3ccc(F)c4c3CCN(C(Cc3cnn(C(F)F)c3)C(=O)NC(C)(C)Cc3nc(C(F)(F)F)cn3-c3ccn(C5CC5)c(=O)c3)C4=O)c(F)cc21 ; Cn1nc(F)c2nc(-c3ccc(F)c4c3CCN(C(Cc3cnn(C(F)F)c3)C(=O)NC(C)(C)Cn3nc(C(F)(F)F)cc3-c3ccn(C5CC5)c(=O)n3)C4=O)c(F)cc21 ; Cn1nc(F)c2nc(-c3ccc(F)c4c3CCN(C(Cc3cnn(C(F)F)c3)C(=O)NC(C)(C)Cn3nc(C(F)(F)F)cc3-c3cnc5nncn5c3)C4=O)c(F)cc21]

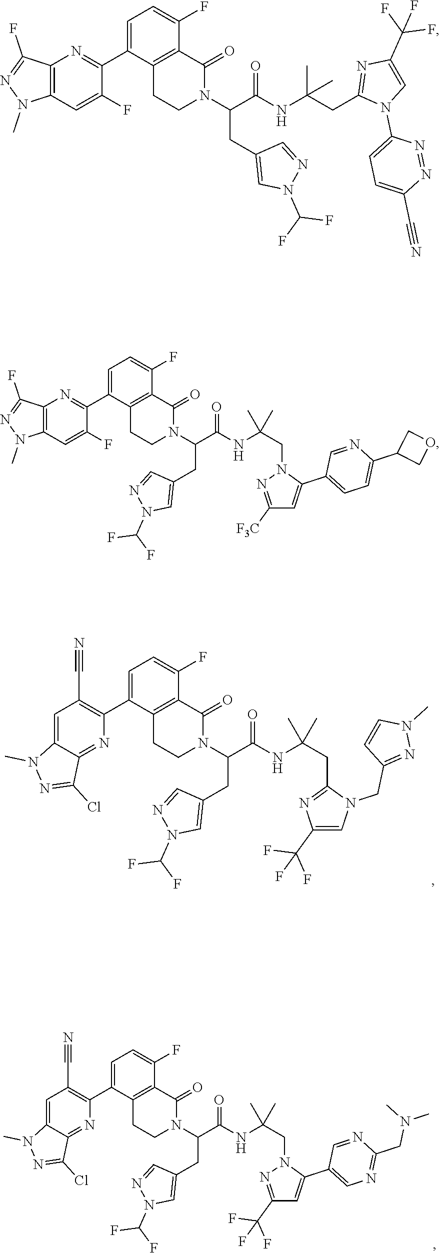

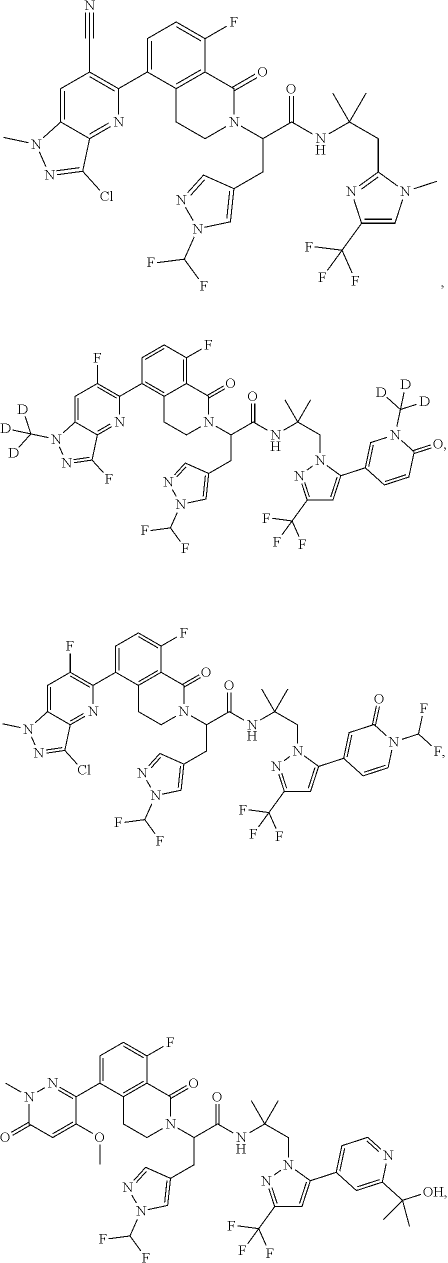

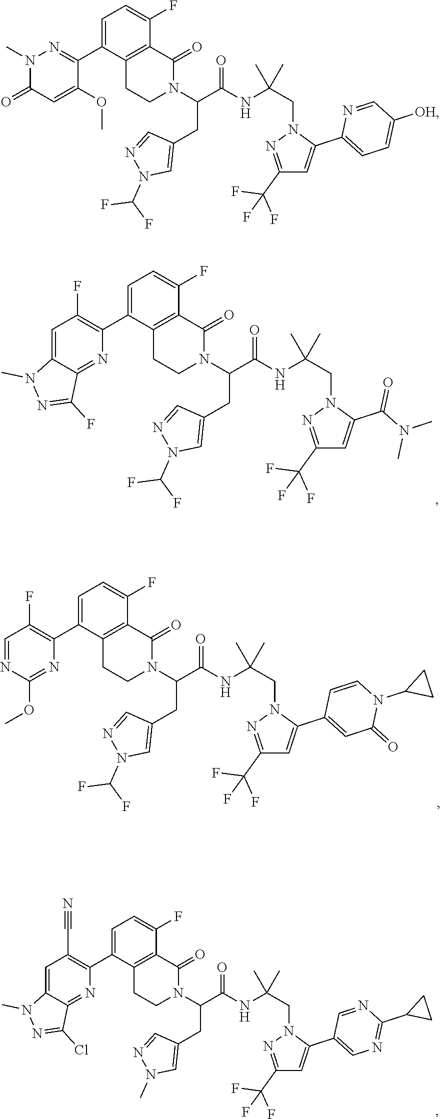

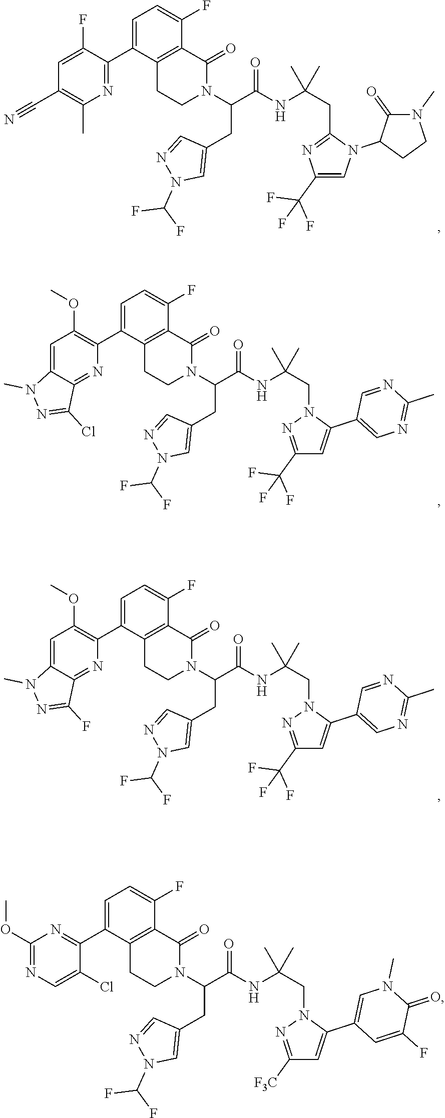

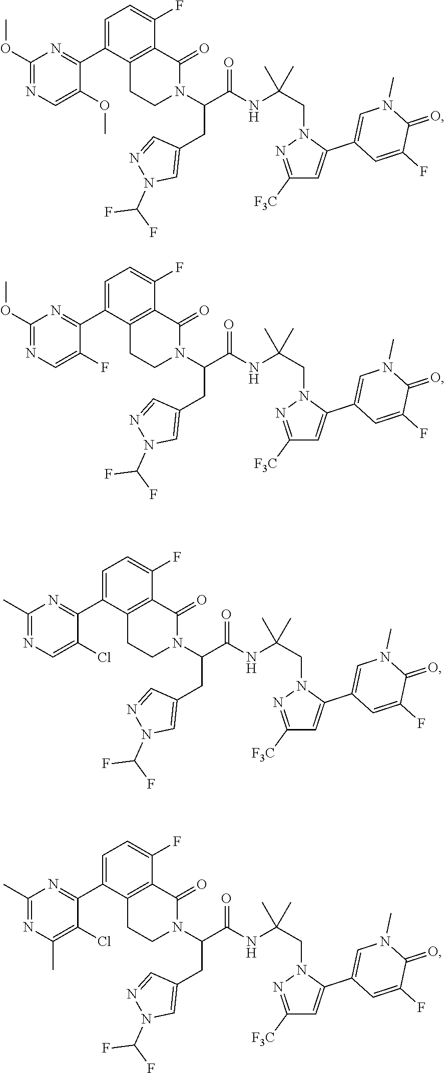 [SMILES: COc1ncc(OC)c(-c2ccc(F)c3c2CCN(C(Cc2cnn(C(F)F)c2)C(=O)NC(C)(C)Cn2nc(C(F)(F)F)cc2-c2cc(F)c(=O)n(C)c2)C3=O)n1 ; COc1ncc(F)c(-c2ccc(F)c3c2CCN(C(Cc2cnn(C(F)F)c2)C(=O)NC(C)(C)Cn2nc(C(F)(F)F)cc2-c2cc(F)c(=O)n(C)c2)C3=O)n1 ; Cc1ncc(Cl)c(-c2ccc(F)c3c2CCN(C(Cc2cnn(C(F)F)c2)C(=O)NC(C)(C)Cn2nc(C(F)(F)F)cc2-c2cc(F)c(=O)n(C)c2)C3=O)n1 ; Cc1nc(C)c(Cl)c(-c2ccc(F)c3c2CCN(C(Cc2cnn(C(F)F)c2)C(=O)NC(C)(C)Cn2nc(C(F)(F)F)cc2-c2cc(F)c(=O)n(C)c2)C3=O)n1]

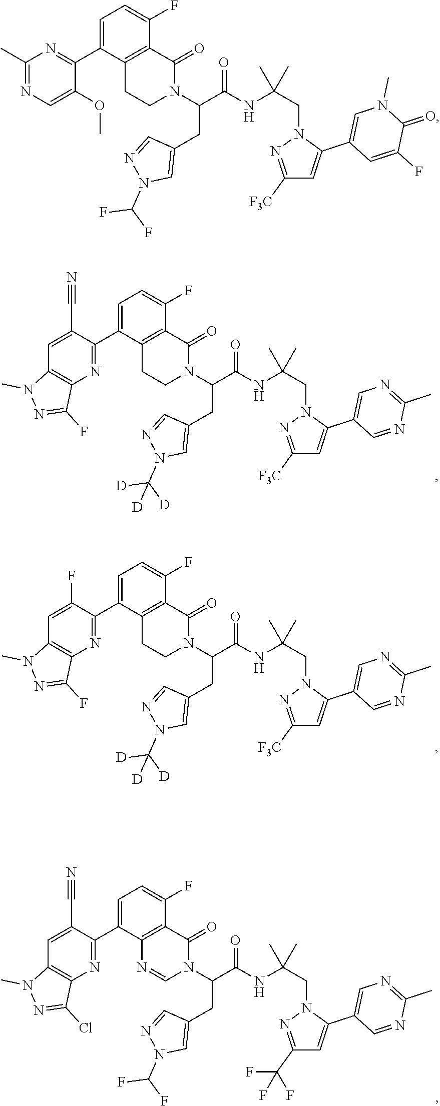

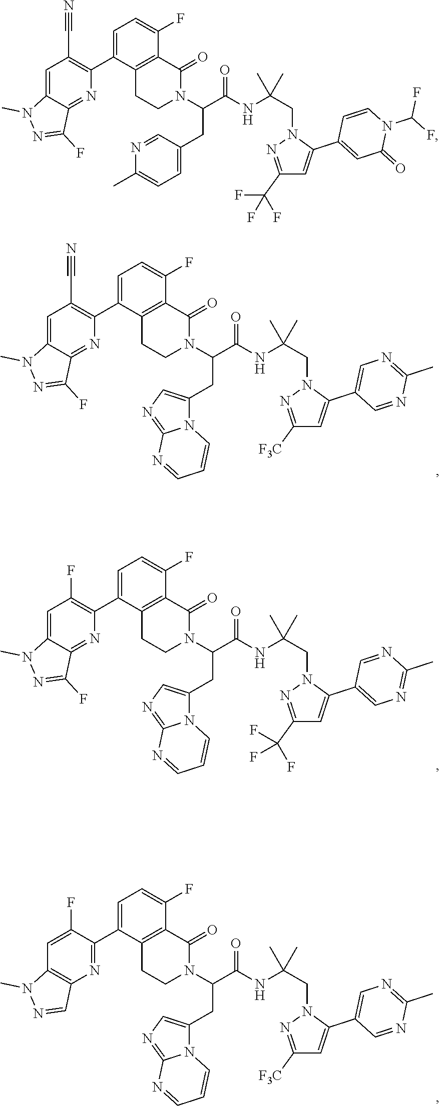

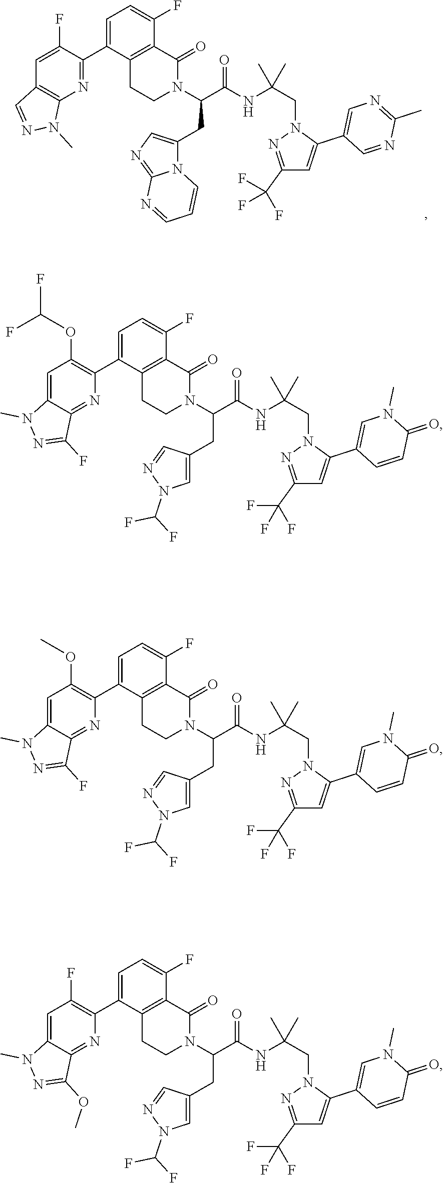

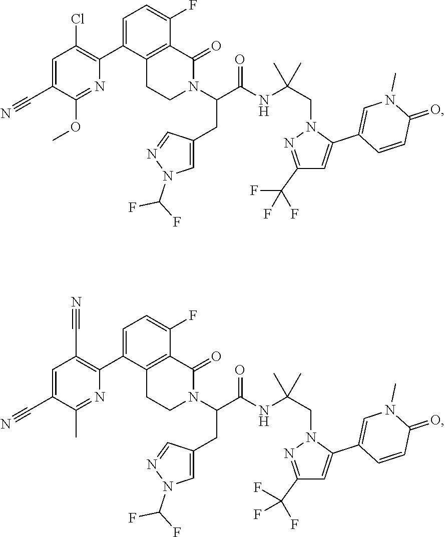 [SMILES: COc1nc(-c2ccc(F)c3c2CCN(C(Cc2cnn(C(F)F)c2)C(=O)NC(C)(C)Cn2nc(C(F)(F)F)cc2-c2ccc(=O)n(C)c2)C3=O)c(Cl)cc1C#N ; Cc1nc(-c2ccc(F)c3c2CCN(C(Cc2cnn(C(F)F)c2)C(=O)NC(C)(C)Cn2nc(C(F)(F)F)cc2-c2ccc(=O)n(C)c2)C3=O)c(C#N)cc1C#N]

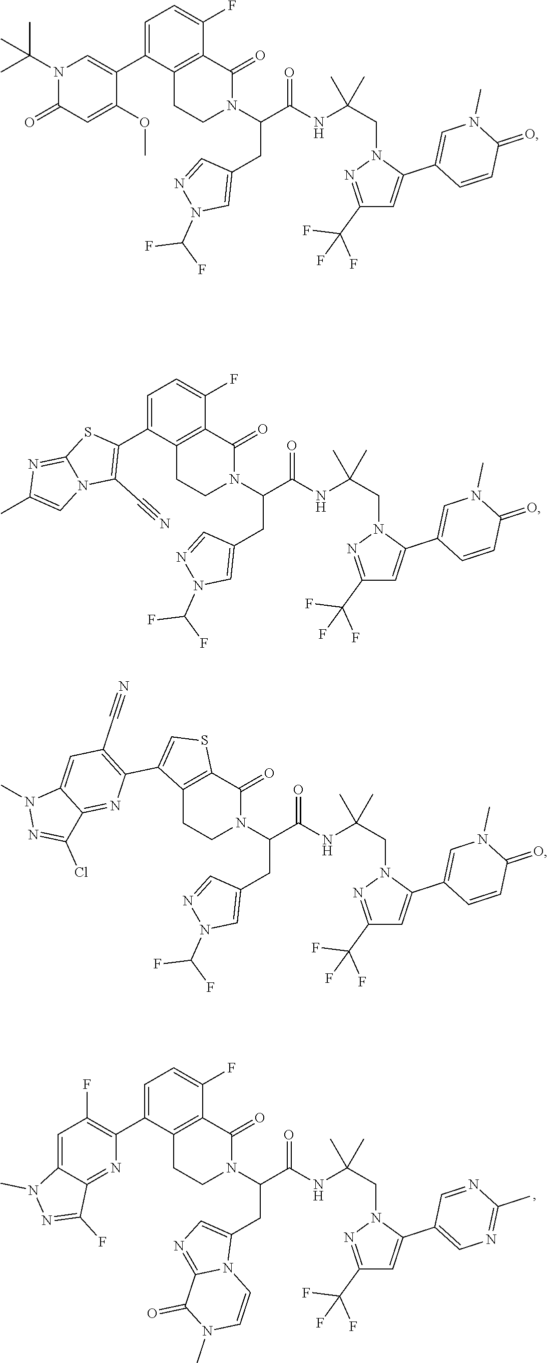

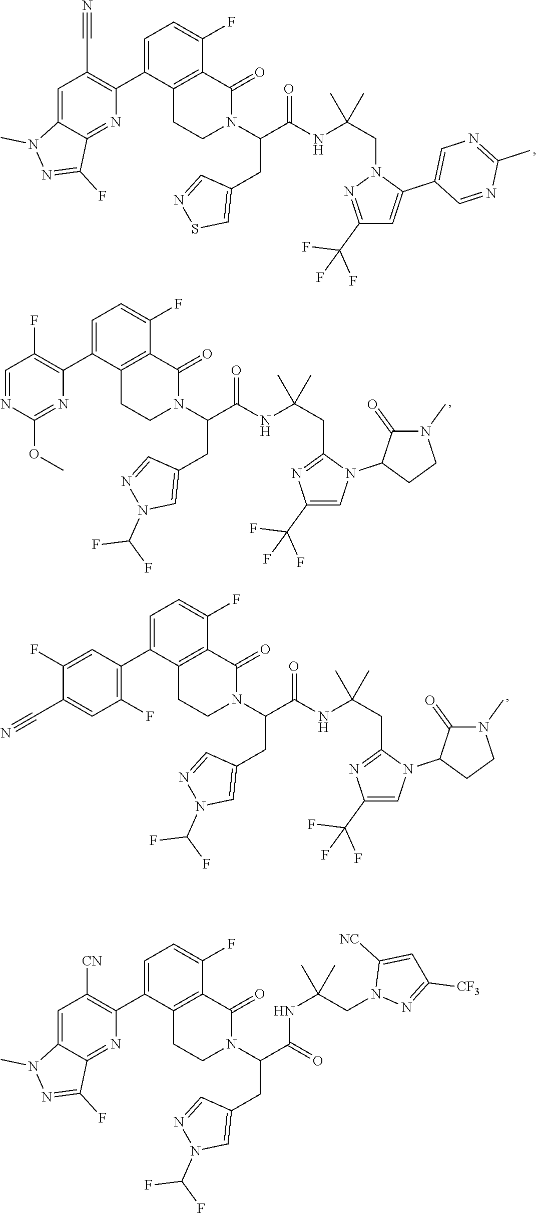

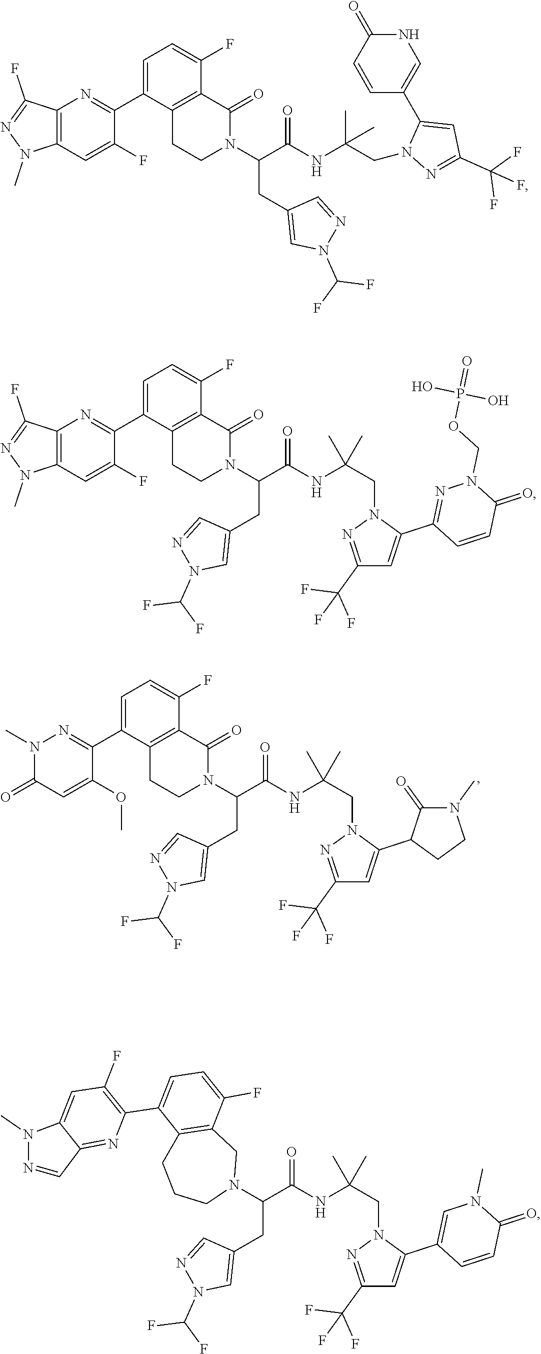

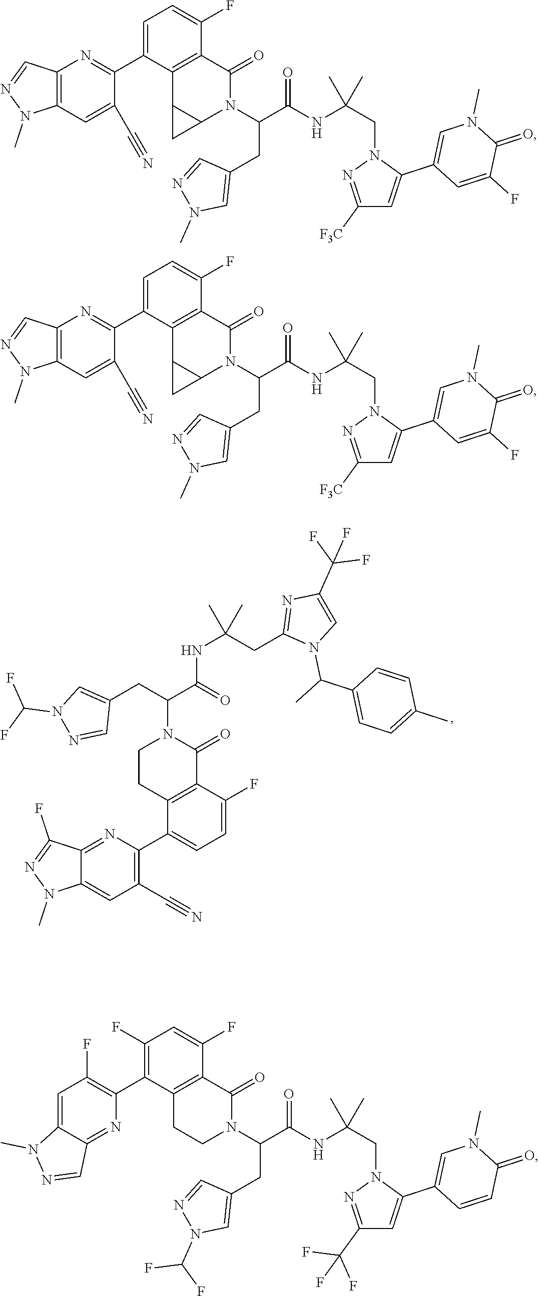

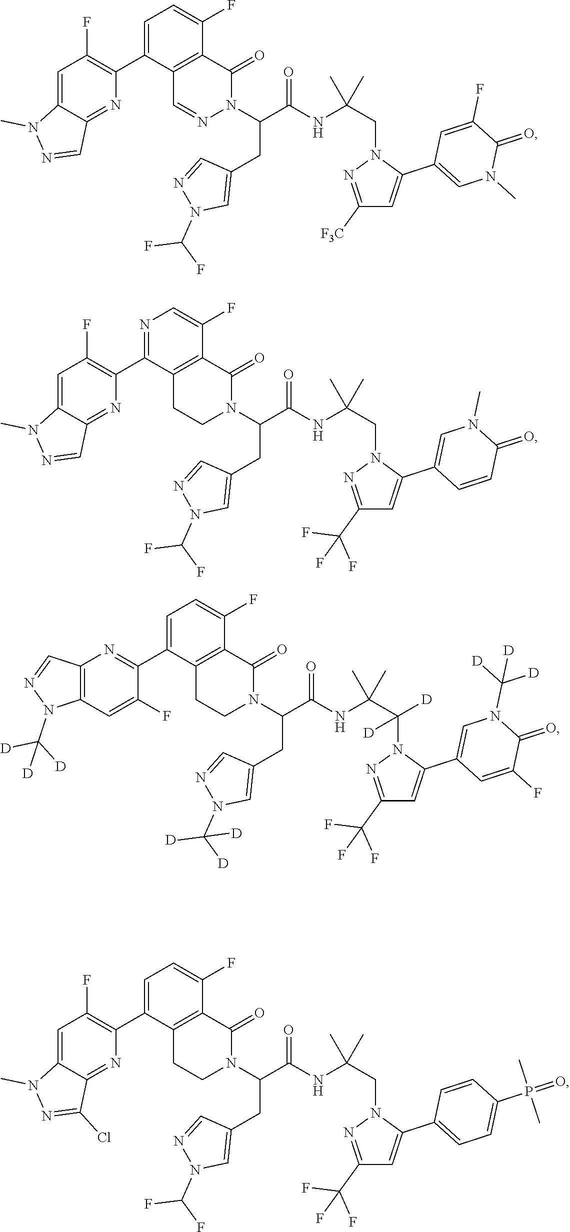

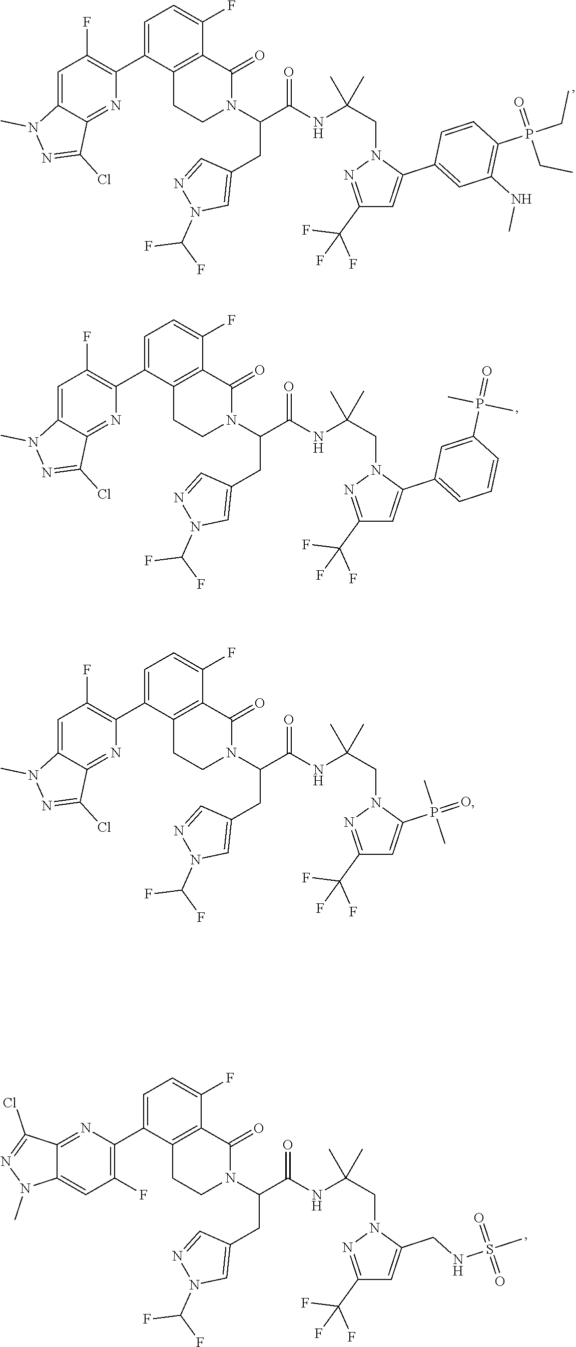

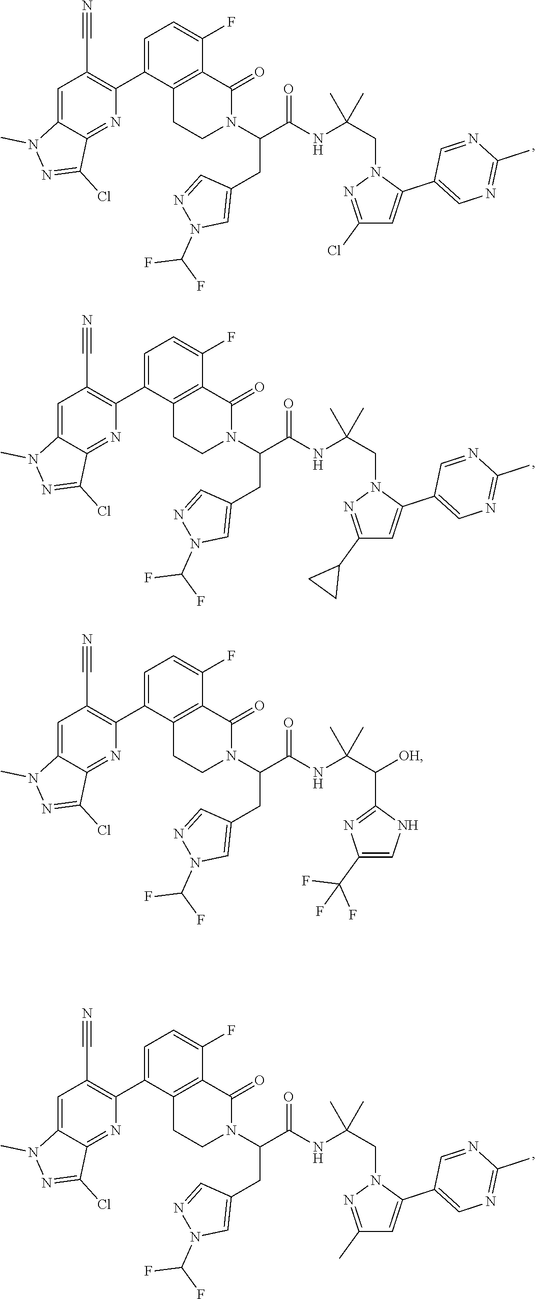

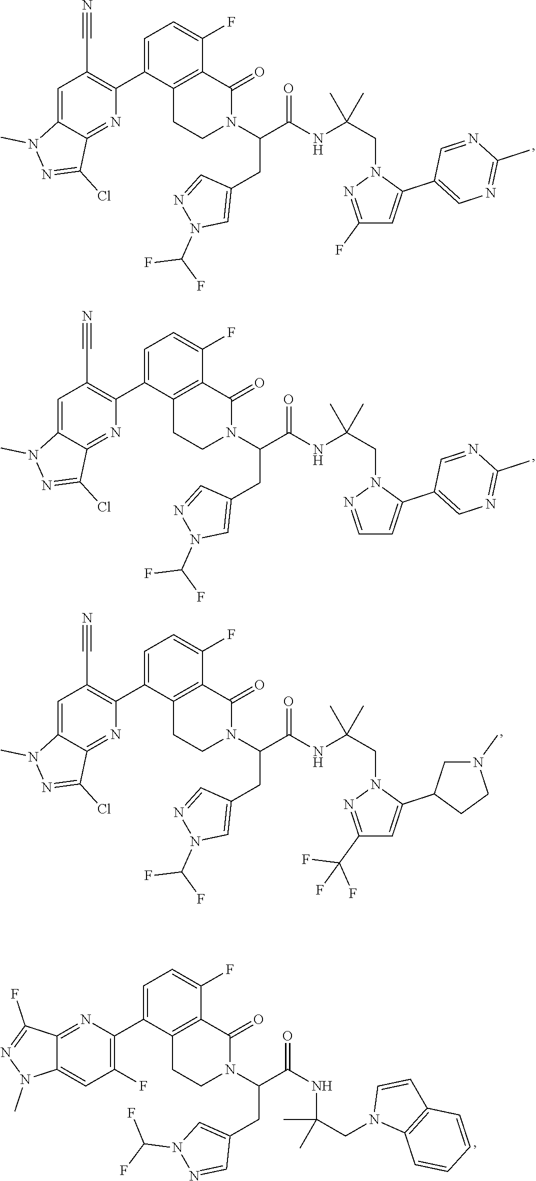

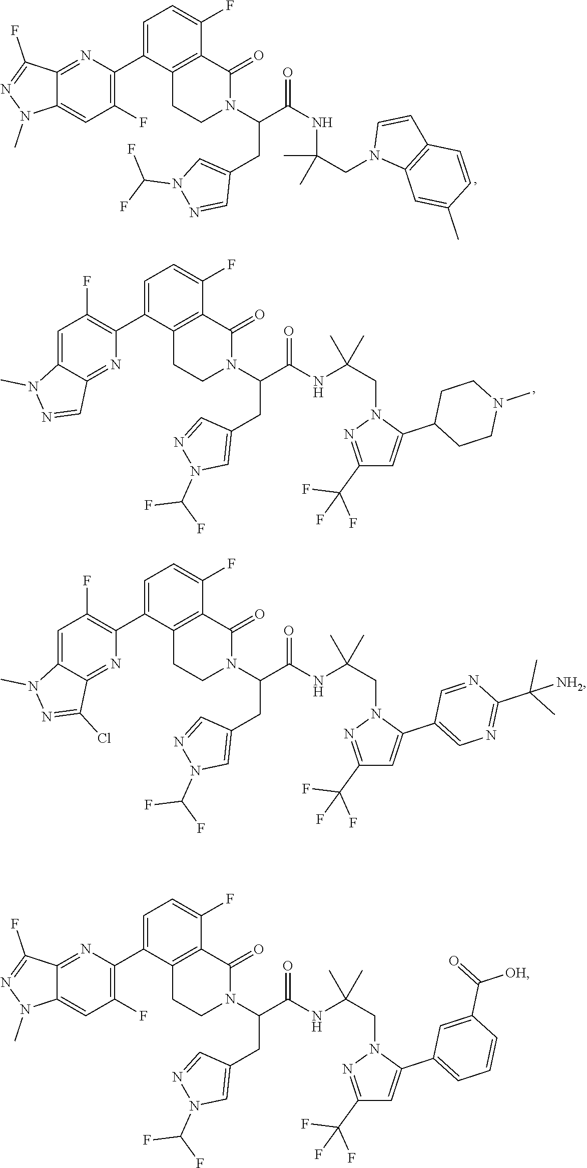

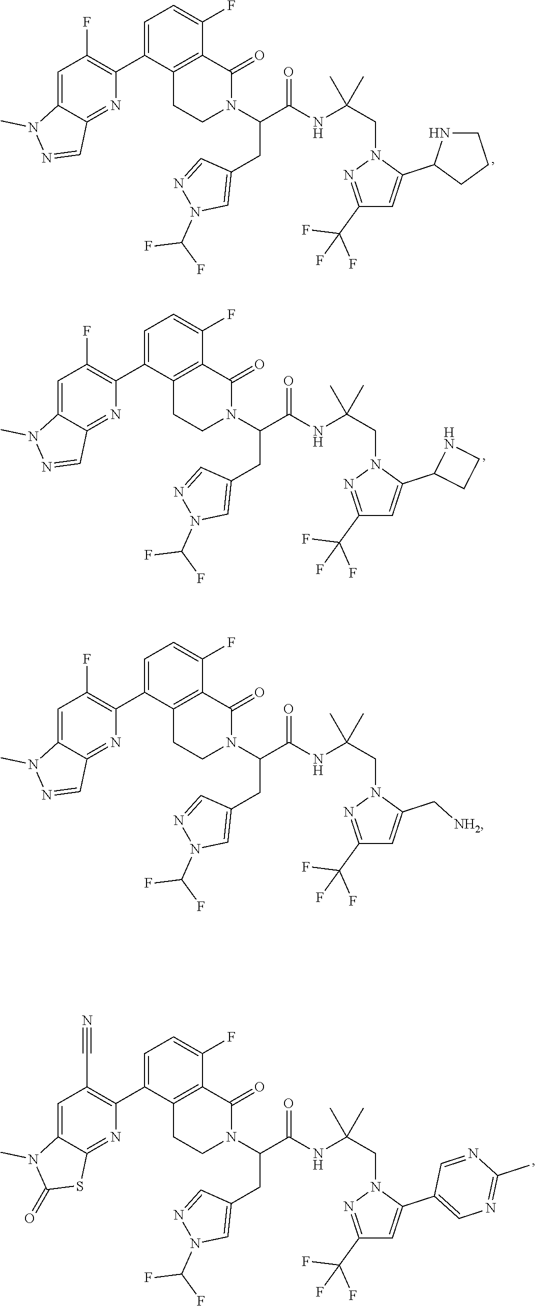

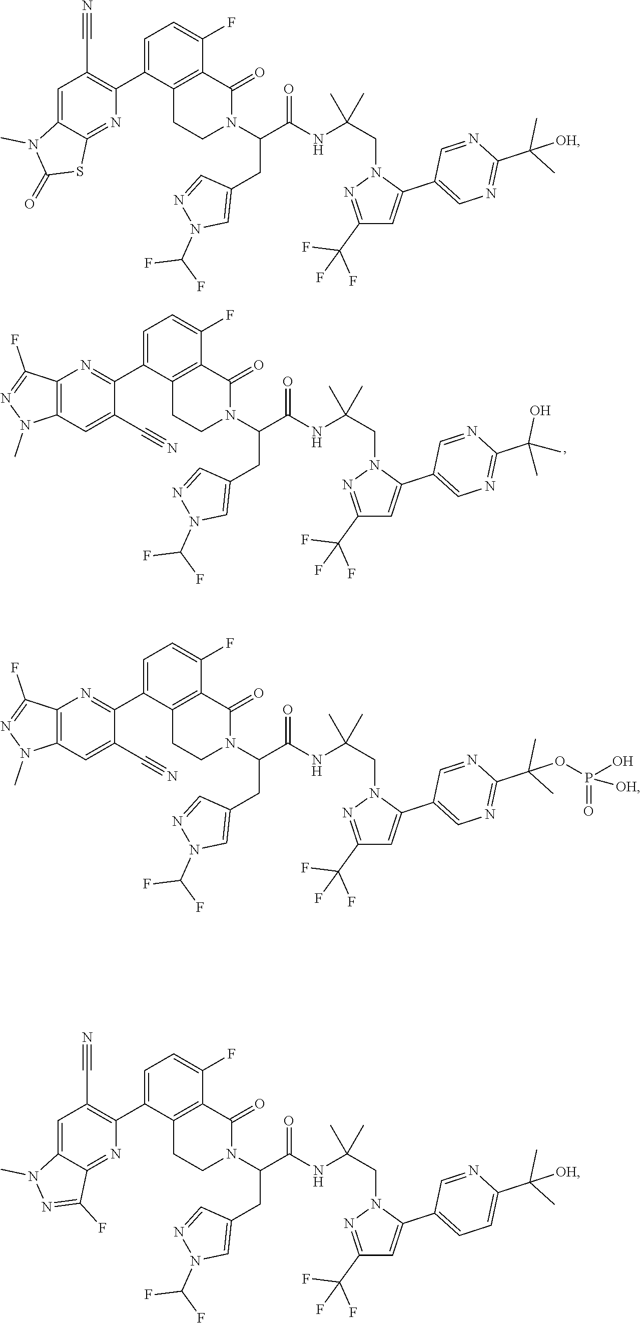

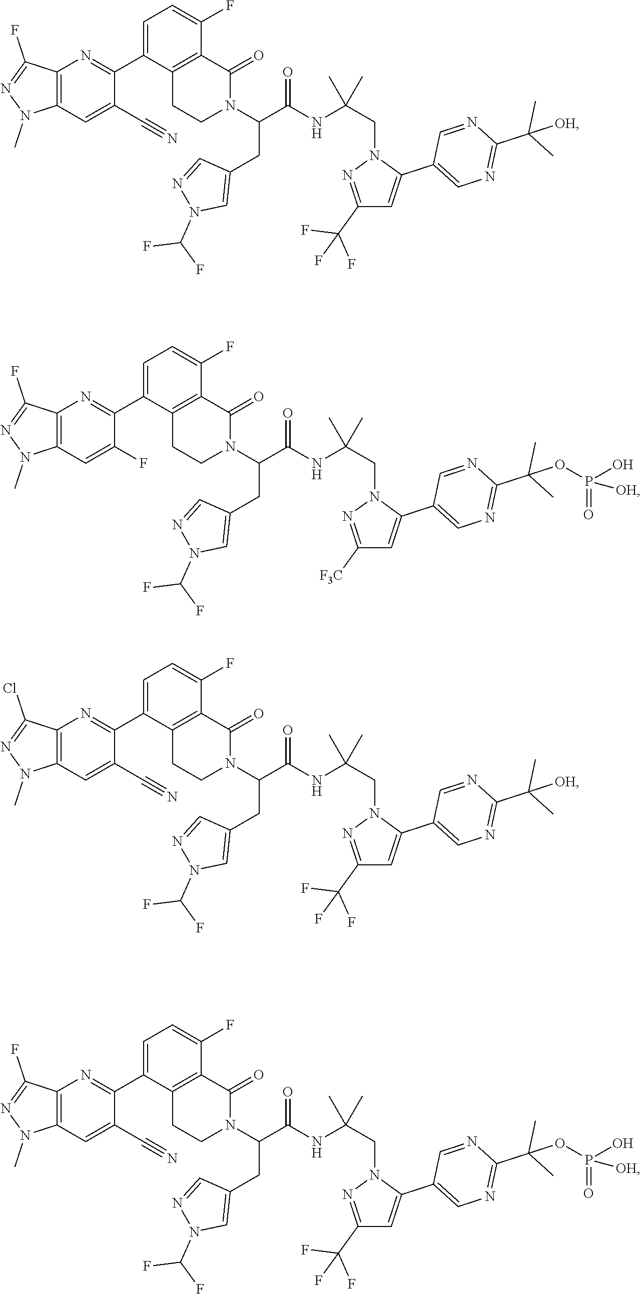 [SMILES: Cn1nc(F)c2nc(-c3ccc(F)c4c3CCN(C(Cc3cnn(C(F)F)c3)C(=O)NC(C)(C)Cn3nc(C(F)(F)F)cc3-c3cnc(C(C)(C)O)nc3)C4=O)c(C#N)cc21 ; Cn1nc(F)c2nc(-c3ccc(F)c4c3CCN(C(Cc3cnn(C(F)F)c3)C(=O)NC(C)(C)Cn3nc(C(F)(F)F)cc3-c3cnc(C(C)(C)OP(=O)(O)O)nc3)C4=O)c(F)cc21 ; Cn1nc(Cl)c2nc(-c3ccc(F)c4c3CCN(C(Cc3cnn(C(F)F)c3)C(=O)NC(C)(C)Cn3nc(C(F)(F)F)cc3-c3cnc(C(C)(C)O)nc3)C4=O)c(C#N)cc21 ; Cn1nc(F)c2nc(-c3ccc(F)c4c3CCN(C(Cc3cnn(C(F)F)c3)C(=O)NC(C)(C)Cn3nc(C(F)(F)F)cc3-c3cnc(C(C)(C)OP(=O)(O)O)nc3)C4=O)c(C#N)cc21]

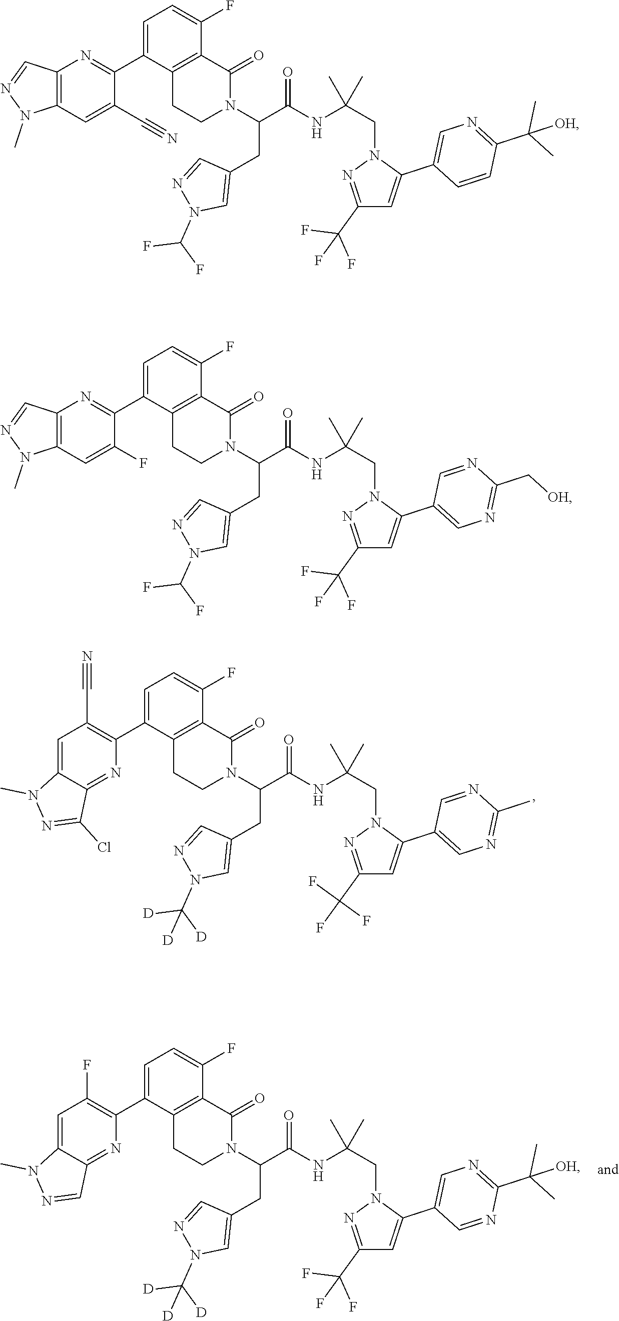

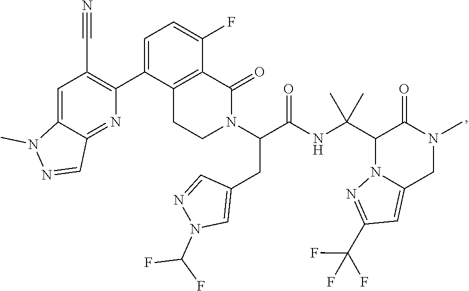

or a pharmaceutically acceptable salt thereof.

### EMBODIMENTS

1. A compound of the formula:

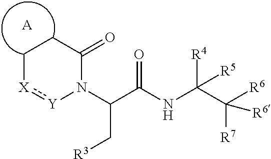

wherein
X is CH₂, OCH₂ or CHCH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl,
X is N, Y is CH and [symbol] is a double bond, or
X is CH, Y is N and [symbol] is a double bond;
A is

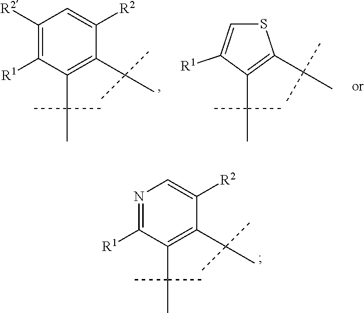

R¹ is a 8-, 9- or 10-membered N-containing bicyclic heterocycle, phenyl or a 5- or 6-membered N-containing heteroaryl, wherein the heterocycle, phenyl or heteroaryl is optionally substituted with 1 to 3 substituents independently selected from:
   CN,
   halo,
   C₁-C₄alkyl optionally substituted with OCH₃,
   C₁-C₄haloalkyl,
   C₁-C₄alkoxy,
   C₁-C₄haloalkoxy,
   CD₃,
   oxo,
   phenyl optionally substituted with 1 or 2 substituents independently selected from halo and OH,
   benzyl optionally substituted with OCH₃,
   NHC(O)pyridyl, wherein the pyridyl is optionally substituted with halo, and a 9-membered N-containing bicyclic heterocycle optionally substituted with CH₃;
R² and R^{2′} are independently H, halo or OCH₃;
R³ is a 5- or 6-membered N-containing heteroaryl optionally substituted with 1 or 2 substituents selected from: CH₃, CD₃, CHF₂, OH and CH₂CN, or a 9-membered N-containing bicyclic heterocycle optionally substituted with 1 or 2 substituents selected from oxo and CH₃; R⁴ and R⁵ are each CH₃ or together form a cyclopropyl;
R⁶ is H, D, OH, CH₃, CO₂R¹⁰,

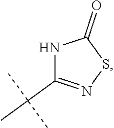

or wherein R¹⁰ is H or C₁-C₄alkyl optionally substituted with OC(O)C₁-C₄alkyl or morpholine, and R^{6′} is H, or
R⁶ is D and R^{6′} is D;
R⁷ is a 5- or 6-membered N-containing heteroaryl or phenyl, wherein the heteroaryl or phenyl is optionally substituted with R⁸ or with R⁸ and R⁹, or wherein the phenyl is optionally substituted with 1 or 2 substituents selected from: halo, CF₃, and pyrazine optionally substituted with CF₂H or OCH₃;
R⁸ is CF₃, CF₂H, halo or cyclopropyl;
R⁹ is:
   i) a 5- or 6-membered heterocycle optionally substituted with 1 to 3 substituents independently selected from:
      halo,
      OH,
      C₁-C₄alkoxy,
      SO₂NH₂,
      a 4- to 6-membered heterocycle optionally substituted with CH₃,
      CN,
      C₃-C₆cycloalkyl,
      CD₃,
      C₁-C₄haloalkyl,
      CR¹²R¹³OP(O)(OH)₂
      oxo, and
      C₁-C₄alkyl, wherein the C₁-C₄alkyl is optionally substituted with OH, OCH₃,
      N(CH₃)₂, a 4- to 6-membered heterocycle or C₃-C₄cycloalkyl optionally substituted
      with halo,
   ii) a 9-membered N-containing bicyclic heterocycle optionally substituted with CN, oxo, C(O)O(CH₃)₃ or OH,
   iii) C₁-C₄alkoxy,
   iv) C₃-C₅cycloalkyl,
   v) halo,
   vi) (CH₂)ₙC(O)R¹¹, wherein n is 0 or 1, R¹¹ is OCH₃, NR¹²R¹³ or a 4- to 6-membered heterocycle,
   vii) C₁-C₄alkyl optionally substituted with a 4- to 6-membered heterocycle or phenyl, which heterocycle or phenyl is optionally substituted with CH₃,
   viii) CN, or
   ix) phenyl optionally substituted with C(O)OH; and
R¹² and R¹³ are independently H or CH₃;
or a pharmaceutically acceptable salt thereof.
   2. The compound according to embodiment 1, wherein A is

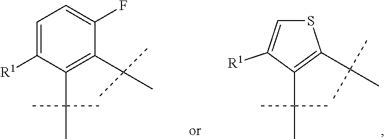

or a pharmaceutically acceptable salt thereof.
3. The compound according to embodiment 1 or embodiment 2, wherein:
X is CH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond, or a pharmaceutically acceptable salt thereof.
   4. The compound according to any one of embodiments 1 to 3, wherein R⁴ and R⁵ are each CH₃, or a pharmaceutically acceptable salt thereof.
   5. The compound according to any one of embodiments 1 to 4, wherein R¹ is: an 8- or 9-membered N-containing bicyclic heterocycle, phenyl or a 6-membered N-containing heteroaryl, wherein the heterocycle, phenyl or heteroaryl is optionally substituted with 1 to 3 substituents independently selected from:
CN,
halo,
C₁-C₄alkyl,
C₁-C₄haloalkyl,
C₁-C₄alkoxy,
C₁-C₄haloalkoxy,
CD₃,
oxo,
benzyl optionally substituted with OCH₃, and
a 9-membered N-containing bicyclic heterocycle optionally substituted with CH₃,
   or a pharmaceutically acceptable salt thereof.
   6. The compound according to embodiment 5, wherein R¹ is:

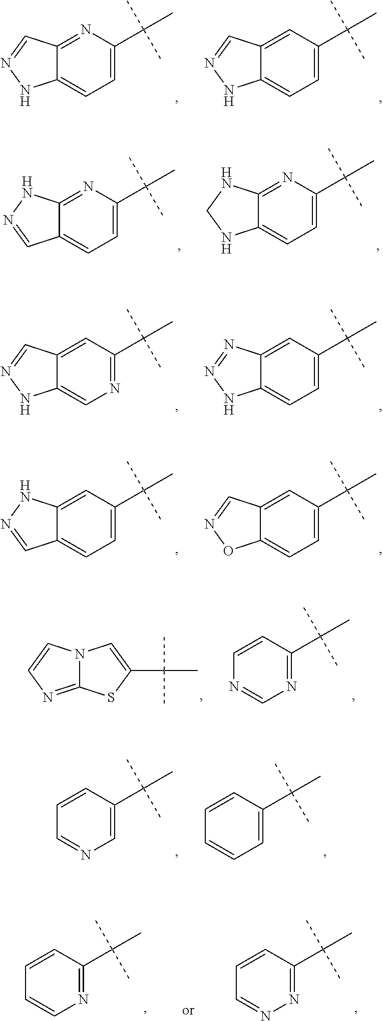

optionally substituted with 1 to 3 substituents independently selected from:
CN,
F,
C₁,
CH₃,
CF₃,
OCH₃,
OCHF₂,
CD₃,
oxo,
benzyl substituted with OCH₃, and
a 9-membered N-containing bicyclic heterocycle substituted with CH₃, or a pharmaceutically acceptable salt thereof.
   7. The compound according to any one of embodiments 1 to 6, wherein R³ is a 5- or 6-membered N-containing heteroaryl optionally substituted with CH₃, CD₃ or CHF₂, or a 9-membered N-containing bicyclic heterocycle optionally substituted with 1 or 2 substituents selected from oxo and CH₃, or a pharmaceutically acceptable salt thereof.
   8. The compound according to embodiment 7, wherein R³ is:

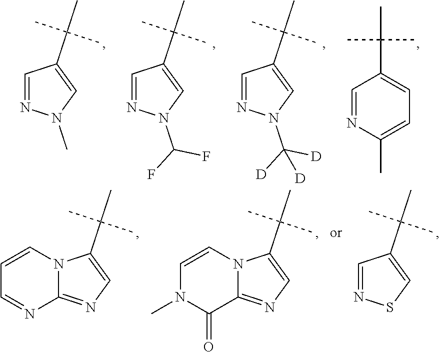

or a pharmaceutically acceptable salt thereof.
9. The compound according to any one of embodiments 1 to 8, wherein R⁶ is H, CO₂R¹⁰, or

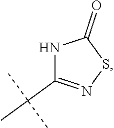

and R^{6′} is H, or a pharmaceutically acceptable salt thereof.
10. The compound according to embodiment 9, wherein R⁶ is H, or a pharmaceutically acceptable salt thereof.
11. The compound according to any one of embodiments 1 to 10, wherein R⁷ is a 5- or 6-membered N-containing heteroaryl or phenyl, wherein the heteroaryl or phenyl is optionally substituted with R⁸ or with R⁸ and R⁹,
R⁸ is CF₃, halo or cyclopropyl; and
R⁹ is:
   i) a 5- or 6-membered heterocycle optionally substituted with 1 to 3 substituents independently selected from:
      halo,
      OH,
      C₁-C₄alkoxy,
      a 4- to 6-membered heterocycle,
      CN,
      C₃-C₆cycloalkyl,
      CD₃,
      C₁-C₄haloalkyl,
      CR¹²R¹³OP(O)(OH)₂
      oxo, and
      C₁-C₄alkyl, wherein the C₁-C₄alkyl is optionally substituted with OH, N(CH₃)₂, or a 4- to 6-membered heterocycle,
   ii) a 9-membered N-containing bicyclic heterocycle,
   iii) C₁-C₄alkoxy,
   iv) C₃-C₅cycloalkyl,
   v) halo,
   vi) (CH₂)ₙC(O)R¹¹, wherein n is 0 or 1, R¹¹ is NR¹²R¹³ or a 4- to 6-membered heterocycle,
   vii) C₁-C₄alkyl, or
   viii) CN,
or a pharmaceutically acceptable salt thereof.
   12. The compound according to embodiment 11, wherein R⁷ is:

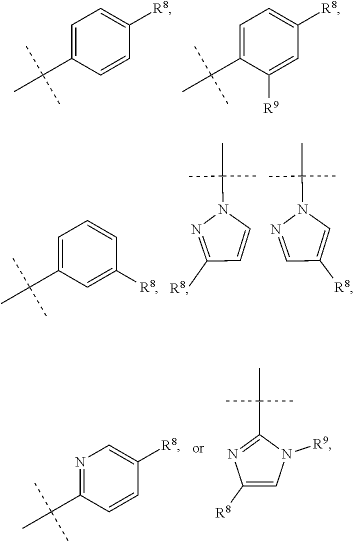

or a pharmaceutically acceptable salt thereof.
13. The compound according to embodiment 1 of the formula:

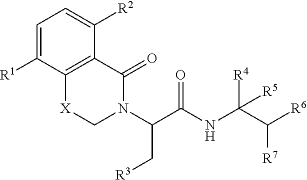

wherein
X is —CH₂—, —OCH₂— or —CH₂CH₂—;
R¹ is:
   i) a 8-, 9- or 10-membered N-containing bicyclic heterocycle optionally substituted with 1 to 3 substituents independently selected from:
      CN,
      halo,
      C₁-C₄alkyl optionally substituted with OCH₃,
      C₁-C₄haloalkyl,
      oxo,
      phenyl optionally substituted with 1 or 2 substituents independently selected from halo and OH, and
      benzyl optionally substituted with OCH₃,
   ii) phenyl or pyridyl optionally substituted with 1 or 2 substituents independently selected from:
      CN,
      NHC(O)pyridyl, wherein the pyridyl is optionally substituted with halo, and a 9-membered N-containing bicyclic heterocycle optionally substituted with CH₃, or
   iii) pyrazolyl optionally substituted with 1 to 3 substituents selected from CN and CH₃;
R² is H, halo or OCH₃;
R³ is a 5- or 6-membered N-containing heteroaryl optionally substituted with 1 or 2 substituents selected from: CH₃, CHF₂, OH and CH₂CN;
R⁴ and R⁵ are each CH₃ or together form a cyclopropyl;
R⁶ is H, OH, CH₃, CO₂R¹⁰, or

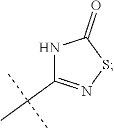

R⁷ is:
   i) phenyl optionally substituted with 1 or 2 substituents selected from:
      halo,
      CF₃, and
      pyrazine optionally substituted with CF₂H or OCH₃, or
   ii) a 5- or 6-membered N-containing heteroaryl optionally substituted with R⁸ or with R⁸ and R⁹;
R⁸ is CF₃, CF₂H, halo or cyclopropyl;
R⁹ is:
   i) a 5- or 6-membered heterocycle optionally substituted with 1 to 3 substituents independently selected from:
      halo,
      OH,
      OCH₃,
      SO₂NH₂,
      a 4- to 6-membered heterocycle,
      oxo, and
      C₁-C₄alkyl, wherein the C₁-C₄alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with CN, oxo, C(O)O(CH₃)₃ or OH,
   iii) C₁-C₄alkoxy,
   iv) C₃-C₅cycloalkyl,
   v) halo, or
   vi) C(O)OCH₃; and
R¹⁰ is H or C₁-C₄alkyl optionally substituted with OC(O)C₁-C₄alkyl or morpholine,
or a pharmaceutically acceptable salt thereof.
   14. The compound according to embodiment 13, wherein R² is F, or a pharmaceutically acceptable salt thereof.
   15. The compound according to embodiment 13 or embodiment 14, wherein X is —CH₂—, or a pharmaceutically acceptable salt thereof.
   16. The compound according to any one of embodiments 13 to 15, wherein R⁴ and R⁵ are each CH₃, or a pharmaceutically acceptable salt thereof.
   17. The compound according to any one of embodiments 13 to 16, wherein R¹ is:
i) a 9-membered N-containing bicyclic heterocycle optionally substituted with 1 to 3 substituents independently selected from:
   CN,
   halo,
   C₁-C₄alkyl,
   oxo, and
   benzyl optionally substituted with OCH₃, or
ii) phenyl optionally substituted with 1 or 2 substituents independently selected from: CN, and
   a 9-membered N-containing bicyclic heterocycle optionally substituted with CH₃, or a pharmaceutically acceptable salt thereof.
      18. The compound according to embodiment 17, wherein R¹ is:

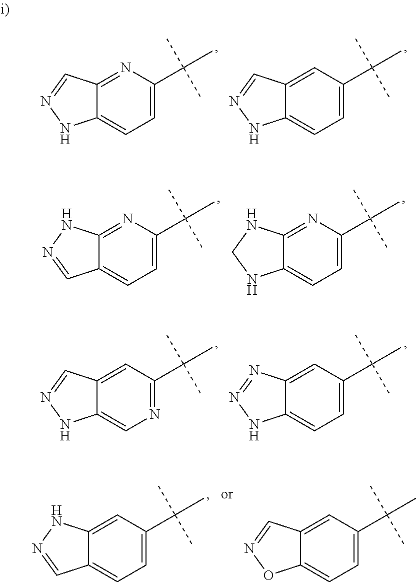

optionally substituted with 1 to 3 substituents independently selected from:
   CN,
   F,
   CH₃,
   oxo, and
   benzyl substituted with OCH₃, or
ii) phenyl substituted with CN and a 9-membered N-containing bicyclic heterocycle substituted with CH₃,
   or a pharmaceutically acceptable salt thereof.
   19. The compound according to any one of embodiments 13 to 18, wherein R³ is a 5-membered N-containing heteroaryl optionally substituted with CH₃ or CHF₂, or a pharmaceutically acceptable salt thereof.
   20. The compound according to embodiment 19, wherein R³ is:

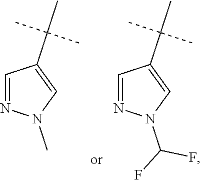

or a pharmaceutically acceptable salt thereof.
21. The compound according to any one of embodiments 13 to 20, wherein R⁶ is H, CO₂R¹⁰, or

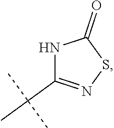

or a pharmaceutically acceptable salt thereof.
22. The compound according to embodiment 21, wherein R⁶ is CO₂R¹⁰ and R¹⁰ is H, CH₂CH₃,

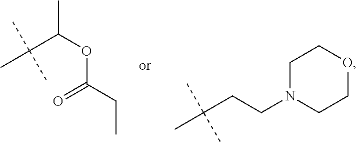

or a pharmaceutically acceptable salt thereof.
23. The compound according to embodiment 21, wherein R⁶ is H, or a pharmaceutically acceptable salt thereof.
24. The compound according to any one of embodiments 13 to 23, wherein R⁷ is:
i) phenyl optionally substituted with halo or CF₃, or
ii) a 5- or 6-membered N-containing heteroaryl optionally substituted with R⁸ or with R⁸ and R⁹,
   wherein
   R⁸ is CF₃, halo or cyclopropyl;
   R⁹ is a 5- or 6-membered heterocycle optionally substituted with 1 to 3 substituents independently selected from:
halo,
OH,
OCH₃,
oxo, and
C₁-C₄alkyl,
   or a pharmaceutically acceptable salt thereof.
   25. The compound according to embodiment 24, wherein R⁷ is:

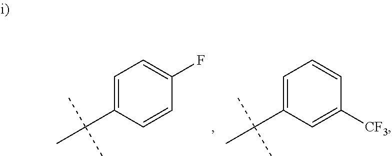

or
ii) a 5- or 6-membered N-containing heteroaryl selected from:

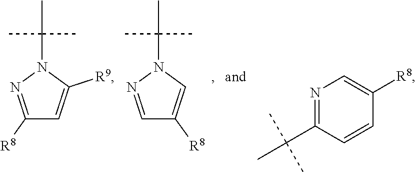

wherein R⁸ is CF₃, F or cyclopropyl, and R⁹ is a 5- or 6-membered heterocycle selected from:

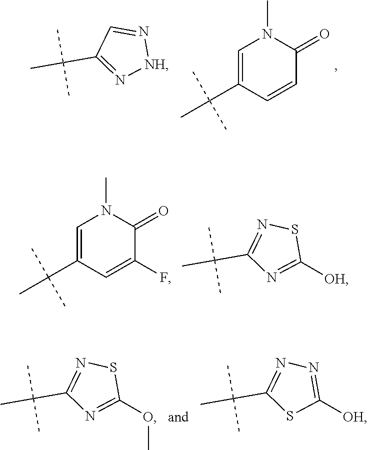

or a pharmaceutically acceptable salt thereof.

In an embodiment, there is provided a pharmaceutical composition comprising a compound of any one of the above formulae, or a pharmaceutically acceptable salt thereof, and at least one of a pharmaceutically acceptable carrier, diluent or excipient. In a preferred embodiment, the pharmaceutical composition is formulated for oral administration.

In an embodiment, there is provided a method for treating type II diabetes mellitus comprising administering to the patient in need thereof a compound of any one of the above formulae, or a pharmaceutically acceptable salt thereof. In another embodiment, there is provided a method for treating type II diabetes mellitus comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In an embodiment, there is provided a method for treating obesity comprising administering to the patient in need thereof a compound of any one of the above formulae, or a pharmaceutically acceptable salt thereof. In another embodiment, there is provided a method for treating obesity comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In a further embodiment, there is provided a method for chronic weight management, a method for improving weight management or a method for providing therapeutic weight loss, comprising administering to the patient in need thereof a compound of any one of the above formulae, or a pharmaceutically acceptable salt thereof. In another embodiment, there is provided a method for chronic weight management, a method for improving weight management or a method for providing therapeutic weight loss comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In a further embodiment, there is provided a method for treating overweight with at least one weight related comorbidity, comprising administering to the patient in need thereof a compound of any one of the above formulae, or a pharmaceutically acceptable salt thereof. In another embodiment, there is provided a method for treating overweight with at least one weight related comorbidity comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided a method for treating a disorder selected from: type 1 diabetes mellitus, metabolic syndrome, dyslipidemia, hepatic steatosis associated with insulin resistance and diabetes, chronic kidney disease (CKD), atherosclerosis, non-alcoholic fatty liver disease (NAFLD), non-alcoholic steatohepatitis (NASH), diabetic kidney disease, a cognitive disorder, heart failure, high blood pressure, obstructive sleep apnea and bone frailty, comprising administering to the patient in need thereof a compound of any one of the above formulae, or a pharmaceutically acceptable salt thereof. In another embodiment, there is provided a method for treating a disorder selected from: metabolic syndrome, dyslipidemia, hepatic steatosis associated with insulin resistance and diabetes, chronic kidney disease (CKD), atherosclerosis, non-alcoholic fatty liver disease (NAFLD), non-alcoholic steatohepatitis (NASH), diabetic kidney disease, a cognitive disorder, heart failure, high blood pressure, obstructive sleep apnea and bone frailty, comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In an embodiment, there is provided a compound of any one of the above formulae, or a pharmaceutically acceptable salt thereof, for use in therapy.

In another embodiment, there is provided a compound of any one of the above formulae, or a pharmaceutically acceptable salt thereof, for use in the treatment of type II diabetes mellitus.

In another embodiment, there is provided a compound of any one of the above formulae, or a pharmaceutically acceptable salt thereof, for use in treating obesity.

In a further embodiment, there is provided a compound of any one of the above formulae, or a pharmaceutically acceptable salt thereof, for use in chronic weight management, improving weight management or providing therapeutic weight loss.

In a further embodiment, there is provided a compound of any one of the above formulae, or a pharmaceutically acceptable salt thereof, for use in treating overweight with at least one weight related comorbidity. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided a compound of any one of the above formulae, or a pharmaceutically acceptable salt thereof, for use in treating a disorder selected from: type 1 diabetes mellitus, metabolic syndrome, dyslipidemia, hepatic steatosis associated with insulin resistance and diabetes, CKD, atherosclerosis, NAFLD, NASH, diabetic kidney disease, a cognitive disorder, heart failure, high blood pressure, obstructive sleep apnea and bone frailty.

In an embodiment, there is provided the use of a compound of any one of the above formulae, or a pharmaceutically acceptable salt thereof, for the manufacture of a medicament for the treatment of type II diabetes mellitus.

In an embodiment, there is provided the use of a compound of any one of the above formulae, or a pharmaceutically acceptable salt thereof, for the manufacture of a medicament for the treatment of obesity.

In a further embodiment, there is provided the use of a compound of any one of the above formulae, or a pharmaceutically acceptable salt thereof, for the manufacture of a medicament for chronic weight management, improving weight management or providing therapeutic weight loss.

In a further embodiment, there is provided the use of a compound of any one of the above formulae, or a pharmaceutically acceptable salt thereof, for the manufacture of a medicament for treating overweight with at least one weight related comorbidity. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided the use of a compound of any one of the above formulae, or a pharmaceutically acceptable salt thereof, for the manufacture of a medicament for the treatment of a disorder selected from: type 1 diabetes mellitus, metabolic syndrome, dyslipidemia, hepatic steatosis associated with insulin resistance and diabetes, CKD, atherosclerosis, NAFLD, NASH, diabetic kidney disease, a cognitive disorder, heart failure, high blood pressure, obstructive sleep apnea and bone frailty.

In an embodiment, there is provided a pharmaceutical composition for treating type II diabetes mellitus comprising an effective amount of a compound of any one of the above formulae, or a pharmaceutically acceptable salt thereof.

In an embodiment, there is provided a pharmaceutical composition for treating obesity comprising an effective amount of a compound of any one of the above formulae, or a pharmaceutically acceptable salt thereof.

In a further embodiment, there is provided a pharmaceutical composition for chronic weight management, improving weight management or providing therapeutic weight loss comprising an effective amount of a compound of any one of the above formulae, or a pharmaceutically acceptable salt thereof.

In a further embodiment, there is provided a pharmaceutical composition for treating overweight with at least one weight related comorbidity comprising an effective amount of a compound of any one of the above formulae, or a pharmaceutically acceptable salt thereof. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided a pharmaceutical composition for treating a disorder selected from: type 1 diabetes mellitus, metabolic syndrome, dyslipidemia, hepatic steatosis associated with insulin resistance and diabetes, CKD, atherosclerosis, NAFLD, NASH, diabetic kidney disease, a cognitive disorder, heart failure, high blood pressure, obstructive sleep apnea and bone frailty, comprising an effective amount of a compound of any one of the above formulae, or a pharmaceutically acceptable salt thereof.

The compounds of any one of the above formulae may be used in simultaneous, separate, or sequential combination with one or more therapeutic agents. Examples of additional therapeutic agents include, but are not limited to, GLP-1 receptor agonist, a glucagon receptor agonist, a dual GLP-1-glucagon receptor agonist, metformin, a thiazolidinedione, a sulfonylurea, a dipeptidyl peptidase 4 inhibitor, a sodium-glucose cotransporter-2 (SGLT-2) inhibitor, a growth differentiation factor 15 modulator (“GDF15”), a peptide tyrosine modulator (“PYY”), a modified insulin, an amylin receptor agonist, a dual amylin-calcitonin receptor agonist and a modified urocortin-2 (UCN-2) agonist.

In a preferred embodiment, the compound of any one of the above formulae is administered orally. In a preferred embodiment, the compound of any one of the above formulae is administered once daily. In another preferred embodiment, the therapeutic use is in a human.

References to Formula III should be understood to refer to compounds of Formula III together with any and all sub-formulae disclosed herein, including Formulae I, Ia, II, IIa, IIIa, IV, IVa, V and Va.

The term “halogen” or “halo” refers to fluorine, chlorine, bromine, or iodine.

The term “C₁-Cₙhalkyl” refers to a straight, or branched chain saturated hydrocarbon containing 1 to n carbon atoms. Examples of a C₁-C₄alkyl group include, but are not limited to, methyl, ethyl, propyl, butyl, and tert-butyl.

The term “C₁-Cₙhaloalkyl” refers to a C₁-Cₙalkyl group, as defined herein, which is substituted with one or more halogen. Examples of C₁-C₄haloalkyl groups include, but are not limited to, trifluoromethyl, difluoromethyl and pentafluoroethyl.

The term “C₁-Cₙalkoxy” refers to a straight, or branched chain saturated hydrocarbon containing 1 to n carbon atoms containing a terminal “O” in the chain, i.e., —O(alkyl). Examples of C₁-C₄alkoxy groups include, but are not limited to, methoxy, ethoxy, propoxy and butoxy.

The term “C₁-Cₙhaloalkoxy” refers to a C₁-Cₙalkoxy group, as defined herein, which is substituted with one or more halogen. Examples of C₁-C₄haloalkoxy groups include, but are not limited to, difluoromethoxy and 2,2-difluoroethoxy.

The term “C₃-Cₙcycloalkyl” refers to a monocyclic saturated carbon ring containing between 3 and n carbon atoms.

The term “heterocycle” refers to an aromatic, saturated or partially saturated ring containing one or more heteroatoms, preferably selected from: N, S and O. An example of a 4-membered heterocycle includes, but is not limited to, oxetane. Examples of 5-membered heterocycles include, but are not limited to, pyrazole, imidazole, triazole, thiophene, oxadiazole and thiadiazole. Examples of 6-membered heterocycles include, but are not limited to, pyridine and pyridazine.

The term “N-containing bicyclic heterocycle” refers to a bicyclic aromatic, saturated or partially saturated ring comprising at least one nitrogen atom and optionally one or more other heteroatoms. An example of an 8-membered N-containing bicyclic heterocycle includes, but is not limited to, dihydropyrroloimidazole. Examples of 9-membered N-containing bicyclic heterocycles include, but are not limited to, pyrazolopyrimidine, imidazopyrimidine, dihydropyrazolopyrazine, dihydropyrrolotriazine and tetrahydropyrazolopyrazine. Examples of 10-membered N-containing bicyclic heterocycles include, but are not limited to, naphthyridine and pyridopyridazine.

The term “heteroaryl” refers to a monocyclic aromatic ring containing one or more heteroatoms, preferably selected from: N, S and O. An “N-containing heteroaryl” comprises at least one nitrogen atom but may optionally comprise one or more other heteroatoms. Examples of 5-membered N-containing heteroaryls include, but are not limited to, pyrazole, imidazole, triazole, oxadiazole and thiadiazole. Examples of 6-membered N-containing heteroaryls include, but are not limited to, pyridine and pyridazine.

The compounds of Formula III provided herein, or a pharmaceutically acceptable salt thereof, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl refers to both C₂H₅ or C₂H₅ where from 1 to 5 hydrogens are replaced by deuterium, such as in C₂DₓH₅₋ₓ. Unless otherwise stated, when an atom is designated specifically as “D” or “deuterium”, the atom is understood to have deuterium at an abundance substantially greater than the natural abundance of deuterium, which is 0.015%.

The term “pharmaceutically acceptable salt” as used herein refers a salt of a compound of the invention considered to be acceptable for clinical and/or veterinary use. Examples of pharmaceutically acceptable salts and common methodologies for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66 (1), 1-19.

A compound herein, or a pharmaceutically acceptable salt thereof, includes all stereoisomers of the compound, for example, an enantiomer, a diastereomer (including cis- and trans-geometric isomer), the racemic form of the isomers, and other mixtures. For example, the compound herein, or a pharmaceutically acceptable salt thereof, may have one or more asymmetric centers.

The term “therapeutically effective amount” refers to the amount or dose of a compound of Formula III, or a pharmaceutically acceptable salt thereof, which, upon single or multiple dose administration to the patient, provides the desired effect in the patient under diagnosis or treatment. The attending physician, as one skilled in the art, can readily determine an effective amount by the use of conventional techniques and by observing results obtained under analogous circumstances. Factors considered in the determination of a therapeutically effective amount or dose of a compound include: whether the compound or its salt will be administered; the co-administration of other agents, if used; the size, age, and general health of the patient; the degree of involvement or the severity of the disorder; the response of the individual patient; the mode of administration; the bioavailability characteristics of the preparation administered; the dose regimen selected; and other relevant circumstances.

As used herein, the terms “treating”, “to treat”, or “treatment”, refers to lowering, reducing, or reversing the progression or severity of an existing symptom, disorder, or condition.

As used herein, the term “patient” includes mammals. The patient is preferably human.

The term “overweight with at least one weight related comorbidity” refers to a disease or condition of being overweight and at least one weight related comorbidity. In a particular embodiment, the comorbidity is selected from cancer, depression, diabetes, dyslipidemia, high blood pressure, high cholesterol, obstructive sleep apnea, osteoarthritis, and heart disease. In one embodiment, a subject being overweight is defined as having a body mass index (BMI) of ≥25 to <30.

The compounds of Formula III can be formulated as pharmaceutical compositions administered by any route which makes the compound bioavailable. Preferably, such compositions are for oral administration. Preferably the pharmaceutical compositions are formulated as a tablet, capsule, or a solution. The tablet, capsule, or solution can include a compound of Formula III in an amount effective for treating a patient in need of treatment. Such pharmaceutical compositions and processes for preparing same are well known in the art (See, e.g., “Remington: The Science and Practice of Pharmacy”, A. Adejare Editor, 23rd Ed., 2020, Elsevier Science).

The compounds of the present invention, or salts thereof, may be prepared by a variety of procedures known to one of ordinary skill in the art, some of which are illustrated in the schemes, preparations, and examples below. The products of each step in the schemes below can be recovered by conventional methods well known in the art, including extraction, evaporation, precipitation, chromatography, filtration, trituration, and crystallization. In the schemes below, all substituents unless otherwise indicated, are as previously defined. The reagents and starting materials are readily available to one of ordinary skill in the art. Without limiting the scope of the invention, the following schemes, preparations, and examples are provided to further illustrate the invention. In addition, one of ordinary skill in the art appreciates that compounds of Formula I may be prepared by using starting material or intermediate with the corresponding desired stereochemical configuration which can be prepared by one of skill in the art.

### Abbreviations

“AIBN” refers to azobisisobutyronitrile
“aq.” refers to aqueous
“BOC” or “Boc” refers to tert-butyloxycarbonyl
“CDCl₃” refers to deuterated chloroform
“CD₃OD” refers to deuterated methanol
“ACN” refers to acetonitrile
“AcOH” refers to acetic acid
“DBU” refers to 1,8-diazabicyclo[5.4.0]undec-7-ene
“DCC” refers to N,N′-dicyclohexylcarbodiimide
“DCE” refers to dichloroethane
“DCM” refers to dichloromethane
“DEA” refers to diethylamine
“DEPBT” refers to diethyl (4-oxo-1,2,3-benzotriazin-3-yl)phosphate
“DIEA” refers to N,N-diisopropylethylamine
“DMA” refers to N,N-dimethylacetamide
“DMEA” refers to N,N-dimethylethylamine
“DMF” refers to N,N-dimethylformamide
“DMSO” refers to dimethyl sulfoxide
“DMSO-d₆” refers to deuterated dimethyl sulfoxide
“ee” refers to enantiomeric excess
“ES-MS” refers to electrospray mass spectrometry
“EtOAc” refers to ethyl acetate
“EtOH” refers to ethanol
“Et₂O” refers to diethyl ether
“equiv” or “eq” refers to equivalents
“FA” refers to formic acid
“h” refers to hours
“HATU” refers to 1-[bis(dimethylamino)methylene]-1H-1,2,3-triazolo[4,5-b]pyridinium 3-oxid hexafluorophosphate
“1H-NMR” refers to proton nuclear magnetic resonance
“Prep-HPLC” refers to preparative high-performance liquid chromatography
“Prep-TLC” refers to preparative thin-layer chromatography
“HOAc” refers to acetic acid
“IPA” refers to isopropanol
“IPAm” refers to isopropylamine
“KOAc” refers to potassium acetate
“MeOH” refers to methanol
“min” refers to minutes
“MTBE” refers to methyl tert-butyl ether
“m/z” refers to mass-to-charge ratio
“NaHMDS” refers to sodium bis(trimethylsilyl)amide
“NBS” refers to N-bromosuccinimide
“NCS” refers to N-chlorosuccinimide
“NH₄OAc” refers to ammonium acetate
“NMP” refers to N-methyl pyrrolidine-2-one
“PE” refers petroleum ether
“psi” refers to pounds per square inch
“RBF” refers to round-bottom flask
“RT” refers to room temperature
“R_{f}” refers to retention factor
“sat.” refers to saturated
“SFC” refers to supercritical fluid chromatography
“(Bu” refers to tert-butyl
“TEA” refers to triethylamine
“TFA” refers to trifluoroacetic acid
“wt %” refers to percentage by weight

### Catalyst Abbreviations:

“cataCXium® A Pd G3” refers to mesylate[(di(1-adamantyl)-n-butylphosphine)-2-(2′-amino-1,1′-biphenyl)]palladium(II) (CAS #1651823-59-4).
“BrettPhos Pd G3” refers to [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate (CAS #1470372-59-8).
“PCy3 Pd G4” refers to (methanesulfonato-κO)[2′-(methylamino-κN)[1,1′-biphenyl]-2-yl-κC](tricyclohexylphosphine)-palladium (CAS #2195390-53-3).
“Pd₂(dba)₃” refers to tris(dibenzylideneacetone)dipalladium (CAS #51364-51-3).
“PdCl₂(dtbpf)” refers to 1,1′-bis(di-tert-butylphosphino)ferrocene palladium dichloride (CAS #95408-45-0)
“Pd(PPh₃)₄” refers to tetrakis(triphenylphosphine)palladium(0) (CAS #14221-01-3).
“Pd(dppf)Cl₂” or “PdCl₂(dppf)” refers to [1,1-bis(diphenylphosphino)ferrocene]dichloropalladium(II) (CAS #72287-26-4).
“Pd(dppf)Cl₂·CH₂Cl₂” refers to [1,1′-bis(diphenylphosphino)ferrocene]dichloropalladium(II), complex with dichloromethane (CAS #95464 May 4).
“Rh-COD-[(R)-MaxPhos]-BF₄” refers to [((R)-tert-butylmethylphosphino) (di-tert-butylphosphino) amine](1,5-cyclooctadiene)rhodium (I) tetrafluoroborate (CAS #1263077-53-7)
“SPhos Pd G3” refers to (2-Dicyclohexylphosphino-2′,6′-dimethoxybiphenyl)[2-(2′-amino-1,1′-biphenyl)]palladium(II) methanesulfonate (CAS #1445085-82-4)
“XantPhos” refers to 4,5-bis(diphenylphosphino)-9,9-dimethylxanthene (CAS #161265-03-8).
“XPhos Pd G3” refers to (2-dicyclohexylphosphino-2′,4′,6′-triisopropyl-1,1′-biphenyl)[2-(2′-amino-1,1′-biphenyl)]palladium(II) methanesulfonate (CAS #1445085-55-1).
“XPhos Pd G4” refers to (SP-4-3)-[Dicyclohexyl[2′,4′,6′-tris(1-methylethyl)[1,1′-biphenyl]-2-yl]phosphine](methanesulfonato-κO)[2′-(methylamino-κN)[1,1′-biphenyl]-2-yl-KC]palladium (CAS #1599466-81-5).

In the following schemes, A, R¹, R², R³, R⁴, R⁵, R⁶, R^{6′}, R⁷, R⁸, R⁹, and X are as defined in Formula III.

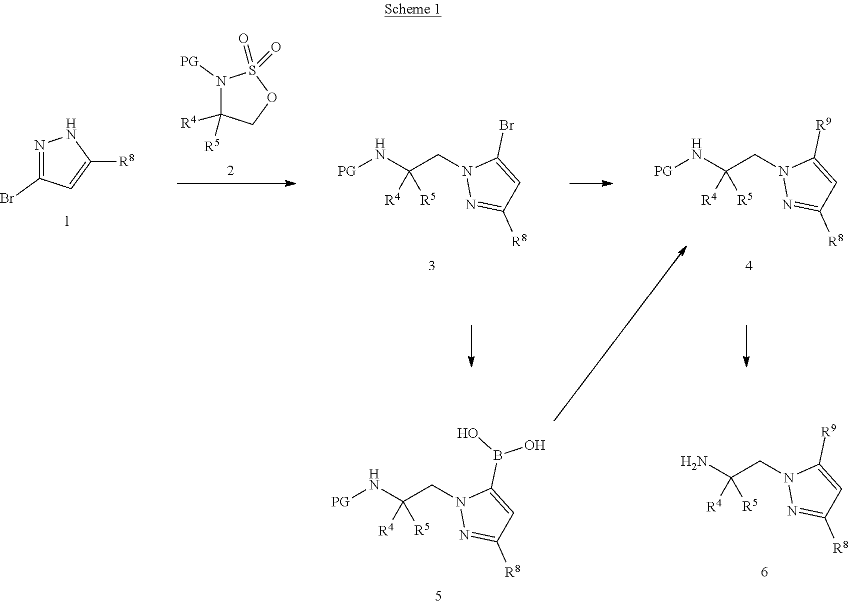

Scheme 1 shows the preparation of intermediate 6, which is useful for the preparation of compounds of the present invention. Imidazole intermediate 1 is reacted with oxathiazolidine intermediate 2 using a carbonate base such as Cs₂CO₃ in a solvent such as DMA at elevated temperature to give intermediate 3. Intermediate 3 then undergoes Suzuki cross-coupling with a R⁹ boronate (R⁹—B(OH)₂ or an ester thereof) using a palladium catalyst, carbonate base, and an aqueous-organic solvent mixture at elevated temperature to give intermediate 4. Alternatively, intermediate 3 is converted to the corresponding boronic acid using tetrahydroxydiboron, a palladium catalyst, a base such potassium acetate, a mixture of solvents such as MeOH and DCE at elevated temperature to give boronic acid 5, which is then coupled with a R⁹ boronate under conditions described above to give intermediate 4. The nitrogen protecting group (represented as “PG” in the above scheme) of intermediate 4 is then removed under conditions known to the person skilled in the art, for example Boc-protecting groups can be removed under acidic conditions (e.g. stirring the Boc-protected intermediate in HCl in 1,4-dioxane at RT) to give 6.

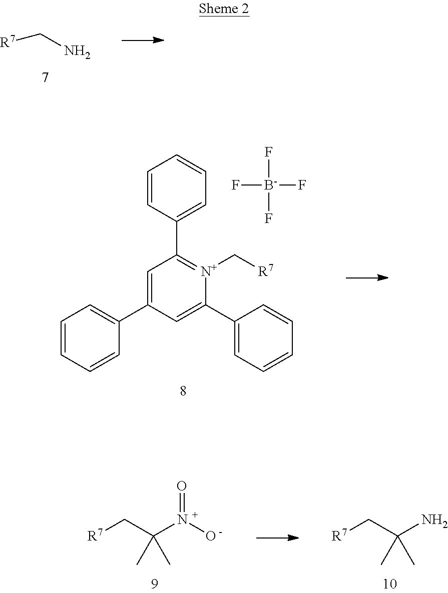

Scheme 2 shows the preparation of Intermediate 10, which is useful for the preparation of compounds of the present invention. Amine 7 is reacted with 2,4,6-triphenylpyrylium tetrafluoroborate in a chlorinated solvent such as DCM to give 8, which is then reacted with 2-nitropropane using an alkoxide base in a solvent such as DMSO at elevated temperature to give intermediate 9. The nitro group is then reduced using Raney® Nickel and hydrogen gas to give amine 10.

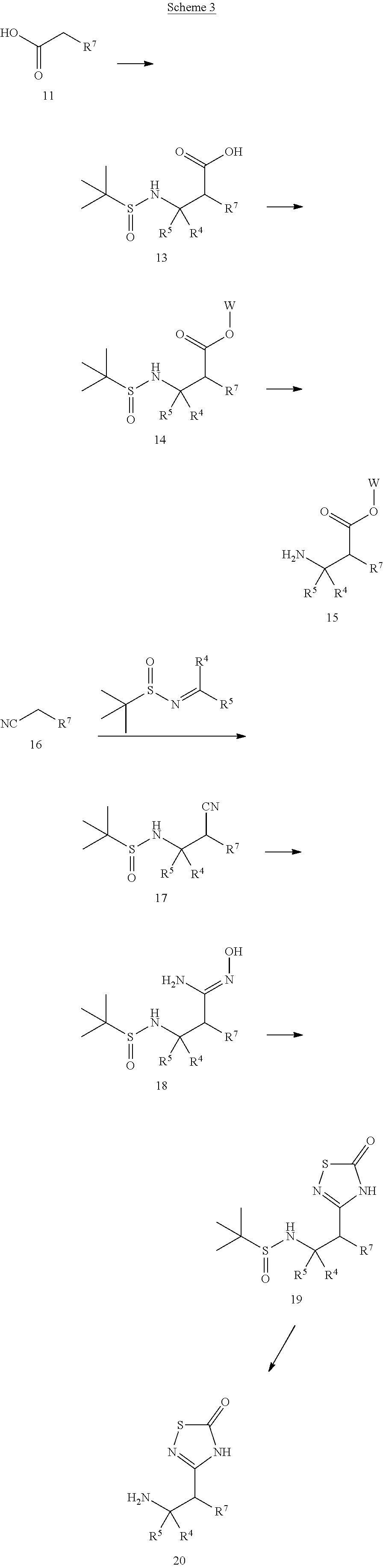

Scheme 3 shows the preparation of intermediates 15 and 20, which are useful for the preparation of compounds of the present invention. Acid intermediate 11 is reacted with lithium diisopropylamide and chlorotitanium triisopropoxide at −70° C., followed by addition of intermediate 12 to give intermediate 13. The acid is then converted to the ester 14, where “W” is an alkyl ester, e.g. by addition of (trimethylsilyl)diazomethane to 13 in an organic solvent giving a methyl ester. Alternatively, the acid in intermediate 11 is protected as an alkyl ester before reaction with intermediate 12, giving intermediate 14 directly. Reaction of intermediate 14 with HCl in a solvent such as 1,4-dioxane at 15° C. gives intermediate 15.

In a similar manner, intermediate 16 is reacted with lithium diisopropylamide and chlorotitanium triisopropoxide at −70° C., followed by addition of intermediate 12 to give intermediate 17. The nitrile is then reacted with hydroxylamine hydrochloride and an organic base at elevated temperature to give intermediate 18, which is then cyclized to thiadiazolone 19 by addition of di-1H-imidazol-1-ylmethanethione at 15° C. in an organic solvent such as THF. Intermediate 19 is then converted to amine 20 under acidic conditions described above.

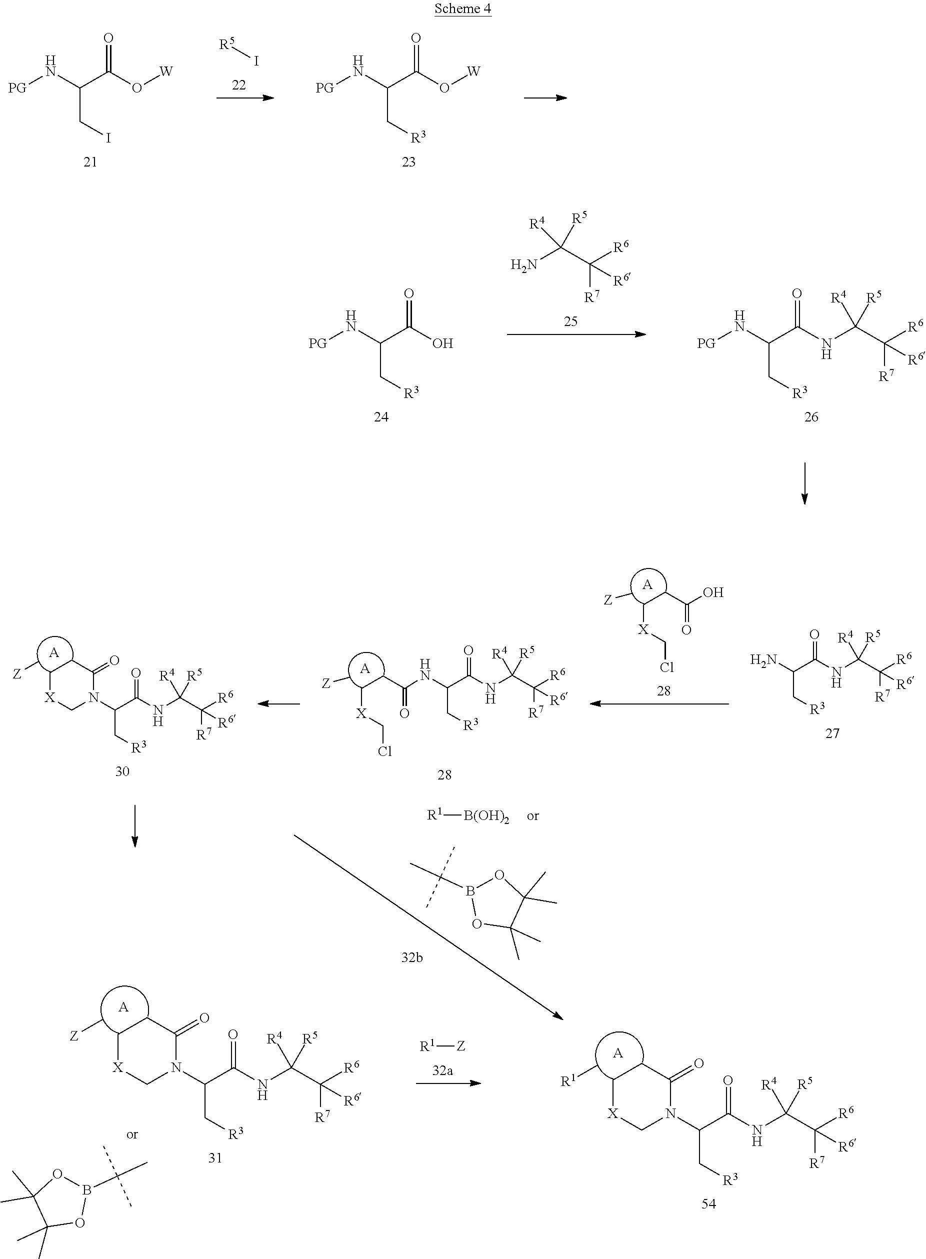

Scheme 4 shows the preparation of compound 54. Alkyl iodide 21 is reacted with aryl/heteroaryl iodide 22 (where “W” is an alkyl group such as methyl or ethyl, and “PG” is a protecting group such as Boc) using pyridine-2,6-bis(carboximidamide)dihydrochloride, nickel (II) chloride, and zinc in a solvent such as DMA to give 23. Aqueous LiOH in a solvent such as THF converts 23 to acid 24, which is then coupled with amine 25 to give 26 under conditions known to a person of skill in the art, e.g. with an amide coupling reagent such as HATU, an organic base such as DIEA, and a polar aprotic solvent such as DMF. The protecting group of intermediate 26 is then removed, e.g. using HCl in 1,4-dioxane and a solvent such as MeOH when “PG” is Boc, giving intermediate 27. Intermediate 27 is then reacted with acid chloride 28 (where “Z” is a halogen) using an organic base such as DIEA in a solvent such as DCM to give 29, which is then cyclized using a carbonate base in a solvent such as ACN at elevated temperature giving intermediate 30. Compound 30 is then converted to the boronic ester 31 using bis(pinacolato)diboron, KOAc, and a palladium catalyst such as Pd(dppf)Cl₂·CH₂Cl₂ in a solvent such as 1,4-dioxane. Alternatively, bromide 30 is converted to the boronic acid using hypodiboric acid, KOAc, and a palladium catalyst such as XPhos Pd G3 at elevated temperature. Finally, boronate 31 undergoes Suzuki cross-coupling with 32a, where “Z” is a halogen, using conditions known to a person skilled in the art e.g. a carbonate base and a palladium catalyst in an aqueous-organic mixture of solvents such as EtOH/water at elevated temperature to give compound 54. Bromide 30 can also be converted to compound 54 in a one-pot procedure in which 30 is first coupled with bis(neopentyl glycolato)diboron using a palladium catalyst such as dichloro{bis[2-(diphenylphosphino)phenyl]ether}palladium(II), a base such as potassium acetate, in a solvent such as 1,4-dioxane at elevated temperature to give a boronic ester in situ. Then the boronic ester is coupled with aryl halide 32a using a second palladium catalyst such as XPhos Pd G4, a base such as K₃PO₄, and water at elevated temperature to give compound 54. Compound 30 can also be reacted with boronate 32b under Suzuki cross-coupling conditions described above to give compound 54.

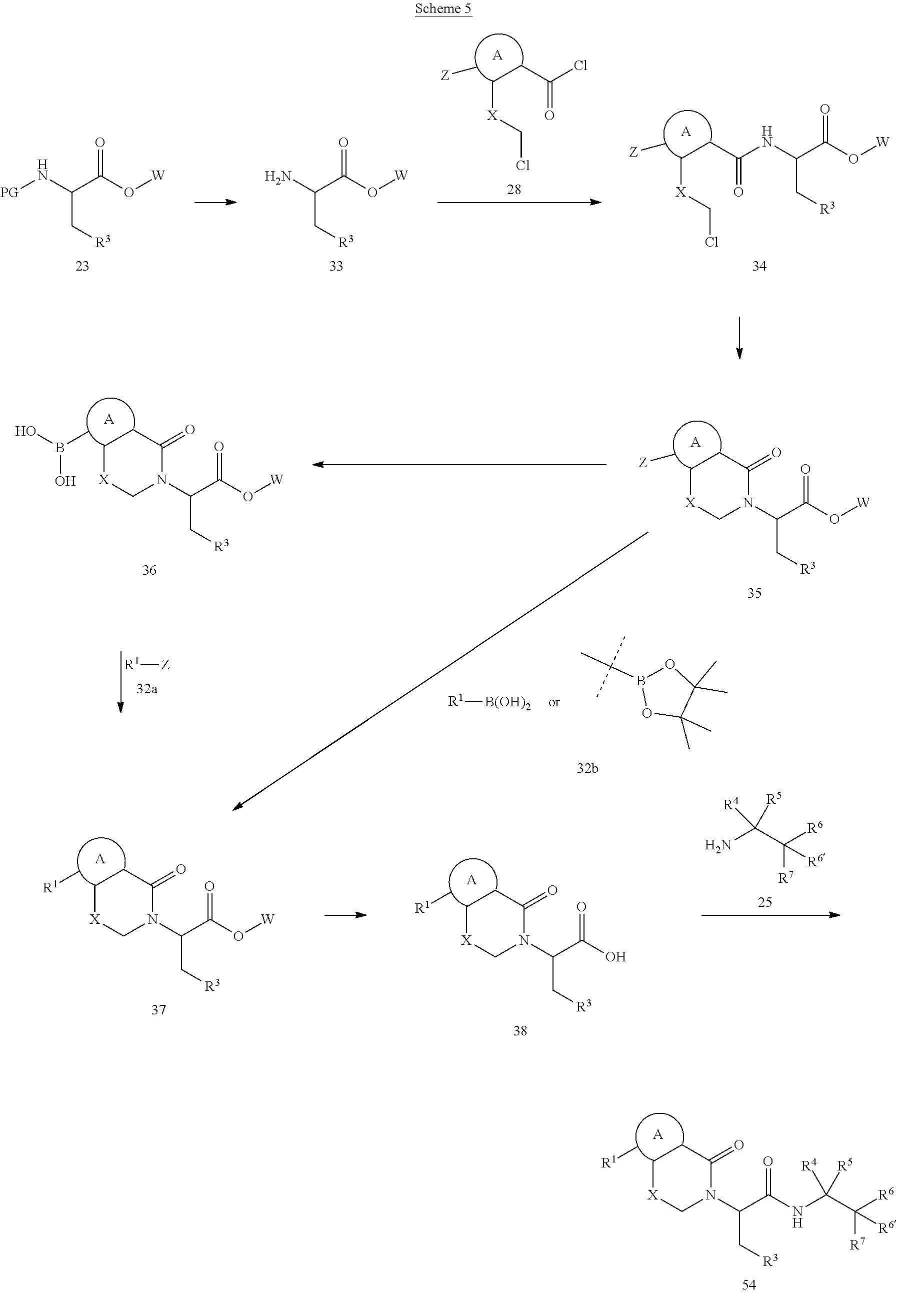

Scheme 5 shows another approach to the preparation of compound 54. Protected amino acid derivative 23 (where “W” is an alkyl group such as methyl or ethyl, and “PG” is a protecting group such as Boc) is first deprotected, for example using HCl in MeOH if “PG” is Boc to give 33, which is then reacted with acid chloride 28 (where Z is a halogen) using an organic base such as TEA in a solvent such as THF to give 34. Intermediate 34 is then cyclized using a carbonate base in a solvent such as ACN giving intermediate 35, which is then converted to the boronic acid using hypodiboric acid, KOAc, and a palladium catalyst such as XPhos Pd G3 at elevated temperature to give boronic acid 36. Boronic acid 36 then undergoes Suzuki cross-coupling with 32a, where “Z” is a halogen, using conditions known to a person skilled in the art e.g. a carbonate base and a palladium catalyst such as XPhos Pd G3 in an aqueous-organic mixture of solvents such as 1,4-dioxane/water at elevated temperature to give 37. Alternatively, 35 undergoes Suzuki cross-coupling with boronate 32b under conditions as described above to give 37. Aqueous LiOH in a solvent such as tert-butanol converts 37 to acid 38, which is then coupled with amine 25 to give compound 54 under conditions known to a person of skill in the art, e.g. with an amide coupling reagent such as DEPBT, an organic base such as N-methylmorpholine, and a polar aprotic solvent such as DMA.

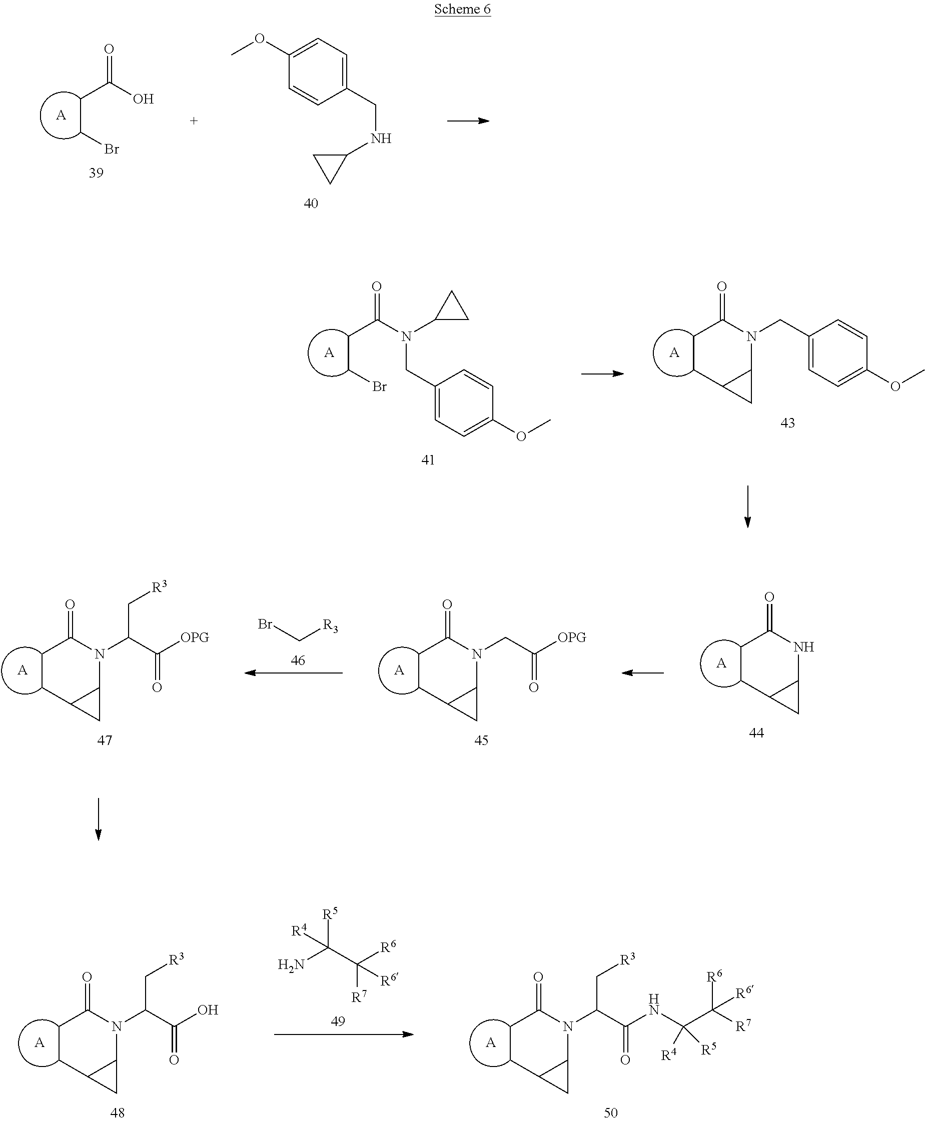

Scheme 6 shows the preparation of compound 50 starting from acid 39, which is coupled to amine 40 using amide coupling conditions well known to the skilled artisan, for example using HATU and an organic base such as TEA in a solvent such as DMF to give 41. Compound 41 is then cyclized using a palladium catalyst such as bis(dibenzylideneacetone)palladium and bis(1,1-dimethylethyl)(methyl)phosphine tetrafluoroborate, a phosphate base such as K₃PO₄, in a solvent such as toluene at elevated temperature to give 43. Subsequent deprotection using TFA and anisole at elevated temperature gives 44, which is then alkylated with ethyl bromoacetate using a carbonate base in a solvent such as DMF at elevated temperature to give 45. Compound 45 is treated with a base such as NaHMDS at −78° C. and then alkylated with alkyl bromide 46 to give 47, which then undergoes ester hydrolysis using aqueous LiOH to give 48. Acid 48 is then coupled with amine 49 under coupling conditions well known to the skilled artisan such as using HATU and TEA to give 50.

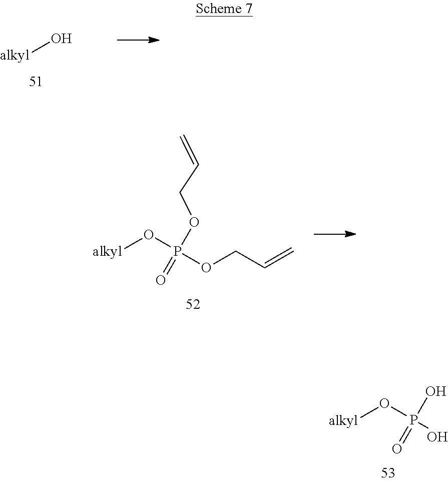

Scheme 7 shows the preparation of alkyl phosphates, where “alkyl-OH” (51) represents an alkyl-hydroxyl substitution in compounds of the present invention. Alcohol 51 is reacted with diallyl n,n-diisopropylphosphoramidite in the presence of 1H-tetrazole in a solvent such as DCM, followed by addition of hydrogen peroxide at 0° C. to give 52. The allyl groups are removed using a palladium catalyst such as Pd(PPh₃)₄ and phenylsilane to give 53.

### Preparation 1

### 6-Chloro-5-iodo-3-nitropyridin-2-amine

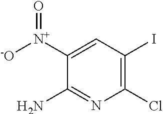

To a mixture of 6-chloro-3-nitropyridin-2-amine (5 g, 28.2 mmol) in H₂O (15 mL) and sulfuric acid (8.45 g, 5.17 mL, 84.7 mmol) was added periodic acid (1.38 g, 5.9 mmol) and the mixture was stirred at 100° C. for 0.5 h under N₂. Iodine (3.29 g, 12.7 mmol) was then added, and the reaction was stirred at 100° C. for an additional 2 h. The reaction mixture was diluted with sat. aq. sodium thiosulfate (100 mL) and filtered to provide the title compound (8 g, 90%) as a yellow solid. ES/MS m/z 299.9 (M+H).

### Preparation 2

### 6-Chloro-5-iodo-N-methyl-3-nitropyridin-2-amine

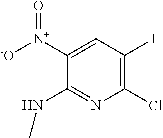

To a solution of 6-chloro-5-iodo-3-nitropyridin-2-amine (3 g, 9.017 mmol) in THF (20 mL) was added NaH (757 mg, 19 mmol) at 0° C. under N₂. The reaction was stirred at 0° C. for 0.5 h and methyl iodide (595 μL, 9.0 mmol) was added. The mixture was stirred for an additional 2 h, then was quenched by addition of sat. NH₄Cl (30 mL) and extracted with EtOAc (30 mL×2). The combined organic layers were washed with sat. aq. NaCl (30 mL), dried over Na₂SO₄ and concentrated. The residue was purified on silica gel, eluting with 0-20% EtOAc in PE to obtain the title compound (1.1 g, 34%) as a yellow solid. ES-MS m/z 314.0 (M+H).

### Preparation 3

### 5-Amino-2-chloro-6-methylnicotinonitrile

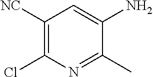

To a solution of 2-chloro-6-methyl-5-nitronicotinonitrile (3.72 g, 18.5 mmol) in EtOH (160 mL) and H₂O (40 mL) was added NH₄Cl (5.19 g, 92.3 mmol) and iron (5.42 g, 92.3 mmol) at 25° C. The reaction was stirred at 80° C. for 2 h, filtered, and washed with hot EtOH (3×20 mL). The filtrate was concentrated under reduced pressure, then taken up in EtOAc (100 mL). The organic layer was washed with sat. aq. NaCl (20 mL), dried over Na₂SO₄, filtered, and concentrated. The residue was purified on silica gel, eluting with 0-5% MeOH in DCM to obtain the title compound (2.7 g, 87%) as a yellow solid. ES-MS m/z 168.2 (M+H).

The compounds in the following table were prepared in similar manner as described in Preparation 3 using the appropriate nitropyridine. Reactants can be added in different orders or in differing equivalency, and purification methods were adjusted to suit the compounds. Such variances would be apparent to one skilled in the art.

TABLE 1

| Preparation | Chemical Name | Structure | ES-MS m/z |
|---|---|---|---|
| 4ª | 6-Chloro-5-iodo-N²- methylpyridine-2,3- diamine | 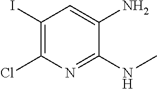 | 284.0 (M + H) |

^{a}The reaction was heated to 50° C. for 16 h.

### Preparation 5

### 6-Bromo-5-fluoro-2-methylpyridin-3-amine

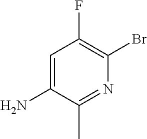

To a solution of 3-amino-5-fluoro-2-methylpyridine (1.0 g, 7.8 mmol) in DMF (20 mL) was added NBS (1.4 g, 7.8 mmol) at 0° C. The mixture was stirred at 20° C. for 16 h, then quenched by adding H₂O (50 mL) and extracted with EtOAc (50 mL×2). The combined organic layers were washed with sat. aq. NaCl (50 mL×2), dried over Na₂SO₄, filtered, and concentrated. The residue was purified on silica gel, eluting with 0-15% EtOAc in PE to obtain the title compound (1.6 g, 96%) as a brown solid. ES-MS m/z 207.0 (M+H).

### Preparation 6

### 1-(5-Bromo-6-fluoro-1H-pyrazolo[4,3-b]pyridin-1-yl)ethan-1-one

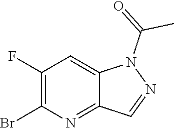

To a mixture of 6-bromo-5-fluoro-2-methylpyridin-3-amine (600 mg, 2.84 mmol) and KOAc (338 mg, 3.41 mmol) in chloroform (12 mL) was added acetic anhydride (812 μL, 8.52 mmol) at 25° C. The reaction was stirred at 60° C. for 2 h, then isoamyl nitrite (576 μL, 4.26 mmol) was added. The mixture was stirred at 60° C. for 16 h, then was filtered and concentrated under reduced pressure. The residue was purified on silica gel, eluting with 0-30% EtOAc in PE to obtain the title compound (130 mg, 15%) as a yellow solid. ES-MS m/z (⁷⁹B/⁸¹Br) 257.8, 259.8 (M+H).

The compounds in the following table were prepared in similar manner as described in Preparation 6 using the appropriate aniline. Reactants can be added in different orders or in differing equivalency, and purification methods were adjusted to suit the compounds. Such variances would be apparent to one skilled in the art.

TABLE 2

| Preparation | Chemical Name | Structure | ES-MS m/z |
|---|---|---|---|
| 7ª | 1-Acetyl-5-chloro-1H- pyrazolo[4,3-b]pyridine- 6-carbonitrile | 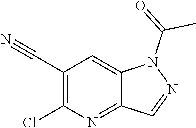 | 221.1 (M + H) |

^{a}Isolated as mixture of desired product and de-acetylated product. The mixture was used without further purification.

### Preparation 8

### 5-Bromo-6-fluoro-1H-pyrazolo[4,3-b]pyridine

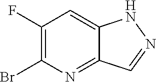 [SMILES: Fc1cc2[nH]ncc2nc1Br]

To 1-(5-bromo-6-fluoro-1H-pyrazolo[4,3-b]pyridin-1-yl)ethan-1-one (106 mg, 390 μmol) in EtOH (3 mL) was added 1M aq. LiOH (468 μL, 468 μmol) at 0° C. The reaction was stirred at 0° C. for 1 h. The pH was adjusted to 7 with FA and the mixture was concentrated, then H₂O (30 mL) was added, and the aqueous layer was extracted with EtOAc (30 mL×2). The combined organic layers were dried over Na₂SO₄, filtered, and concentrated under reduced pressure to give the title compound (107 mg, 98%, 77% Purity) as a yellow solid. ES-MS m/z (⁷⁹B/⁸¹Br) 215.8, 217.8 (M+H).

The compounds in the following table were prepared in similar manner as described in Preparation 8 using the appropriate N-acetyl indazole. Reactants can be added in different orders or in differing equivalency, and purification methods were adjusted to suit the compounds. Such variances would be apparent to one skilled in the art.

TABLE 3

| Preparation | Chemical Name | Structure | ES-MS m/z |
|---|---|---|---|
| 9 | 5-Chloro-1H- pyrazolo[4,3-b]pyridine- 6-carbonitrile | 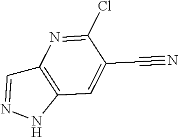 [SMILES: N#Cc1cc2[nH]ncc2nc1Cl] | 179.1 (M + H) |

### Preparation 10

### tert-Butyl (6-chloro-5-fluoropyridin-3-yl)carbamate

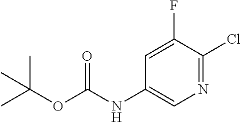 [SMILES: CC(C)(C)OC(=O)Nc1cnc(Cl)c(F)c1]

To a mixture of 5-bromo-2-chloro-3-fluoropyridine (5.00 g, 23.8 mmol) in 1,4-dioxane (150 mL) was added XantPhos (550 mg, 950 μmol), tert-butyl carbamate (3.06 g, 26.1 mmol), Cs₂CO₃ (15.5 g, 47.5 mmol) and Pd₂(dba)₃ (644 mg, 713 μmol) at 20° C. The reaction was stirred at 85° C. for 12 h under N₂, then was filtered and the filter cake was washed with DCM (100 mL×3). The filtrate was concentrated under reduced pressure. The residue was purified on silica gel, eluting with 0-19% EtOAc in PE to obtain the title compound (3.9 g, 62%) as a yellow solid. ES-MS m/z 246.8 (M+H).

### Preparation 11

### tert-Butyl (6-chloro-5-fluoro-4-methylpyridin-3-yl)carbamate

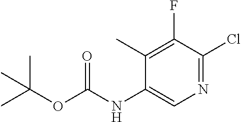 [SMILES: Cc1c(NC(=O)OC(C)(C)C)cnc(Cl)c1F]

To a solution of tert-butyl (6-chloro-5-fluoropyridin-3-yl)carbamate (500 mg, 1.82 mmol) in THF (10 mL) under N₂ at −78° C. was added n-butyllithium (2.5 M in hexane, 1.82 mL, 4.56 mmol) dropwise. The reaction was stirred at −78° C. for 2 h under N₂, then iodomethane (183 μL, 2.92 mmol) in THF (2 mL) was added dropwise. The mixture was stirred for an additional 3 h at −78° C. then poured into ice water (50 mL) and extracted with EtOAc (50 mL×2). The combined organic layers were washed with sat. aq. NaCl (50 mL×2), dried over anhydrous Na₂SO₄, filtered and concentrated. The residue was purified on silica gel, eluting with 0-14% EtOAc in PE to obtain the title compound (405 mg, 77%) as a white solid. ES-MS m/z 260.9 (M+H).

### Preparation 12

### 6-Chloro-5-fluoro-4-methylpyridin-3-amine hydrochloride

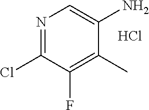

To a solution of tert-butyl (6-chloro-5-fluoro-4-methylpyridin-3-yl)carbamate (405 mg, 1.40 mmol) in MeOH (5 mL) was added 2M HCl in dioxane (6.99 mL, 14.0 mmol). The mixture was stirred at 19° C. for 2 h under N₂, then concentrated to obtain the title compound (270 mg, 88%) as a white solid. ES-MS m/z 158.9 (M+H).

### Preparation 13

### 5-Amino-2-bromo-4-methylbenzonitrile

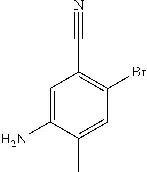

To a solution of 3-amino-4-methylbenzonitrile (5 g, 0.04 mol) in ACN (75 mL) at 0° C. was added NBS (7 g, 0.04 mol) over 2 min. The reaction was allowed to stir for 15 min. The solid was filtered and washed with 30 mL cold ACN, then dried under vacuum to obtain the title compound (5.25 g, 70%) as an off-white solid ES-MS m/z (⁷⁹Br/⁸¹Br) 211.0/213.0.

### Preparation 14

### 5-Chloro-4-fluoro-1H-pyrazolo[3,4-c]pyridine

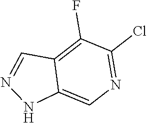 [SMILES: Fc1c(Cl)ncc2[nH]ncc12]

To a solution of 6-chloro-5-fluoro-4-methylpyridin-3-amine hydrochloride (170 mg, 777 μmol) in HOAc (2 mL) was added sodium nitrite (53.6 mg, 777 μmol). The reaction was stirred at RT for 12 h, poured into aq. NaHCO₃ (50 mL), and the aqueous layer was extracted with EtOAc (50 mL×2). The combined organic layers were washed with sat. aq. NaCl (50 mL×2), dried over anhydrous Na₂SO₄, filtered and concentrated under reduced pressure. The residue was purified on silica gel, eluting with 0-30% EtOAc in PE to obtain the title compound (90 mg, 57%) as a white solid. ES-MS m/z 171.9 (M+H).

The compounds in the following table were prepared in a similar manner as described in Preparation 14 using the appropriate aniline. Reactants can be added in different orders or in differing equivalence, and purification methods were adjusted to suit the compounds. Such variances would be apparent to one skilled in art.

TABLE 4

| Preparation | Chemical Name | Structure | ES-MS m/z |
|---|---|---|---|
| 15ª | 5-Bromo-1H-indazole-6- carbonitrile | 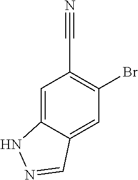 [SMILES: N#Cc1cc2[nH]ncc2cc1Br] | 219.8 (M − H) |

^{a}The reaction was stirred for 30 min instead of 12 h, and the product was purified by trituration with Et₂O.

### Preparation 16

### 5-Bromo-6-fluoro-1-(tetrahydro-2H-pyran-2-yl)-1H-pyrazolo[4,3-b]pyridine

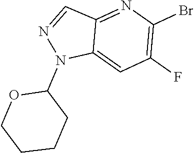 [SMILES: Fc1cc2c(cnn2C2CCCCO2)nc1Br]

To a mixture of 5-bromo-6-fluoro-1H-pyrazolo[4,3-b]pyridine (107 mg, 381 μmol) in THF (5 mL) was added dihydropyran (54.8 μL, 572 μmol) and p-toluenesulfonic acid (6.77 mg, 38.1 μmol) at 20° C. The reaction mixture was stirred at 50° C. for 16 h and concentrated under reduced pressure to give the crude product which was purified by preparative-TLC (PE:EtOAc=5:1, R_{f}=0.5) to obtain the title compound (87 mg, 73%) as a yellow oil. ES-MS m/z (⁷⁹B/⁸¹Br) 300.1, 302.1 (M+H).

The compounds in the following table were prepared in similar manner as described in Preparation 16 from the appropriate NH indazole. Reactants can be added in different orders or in differing equivalency, and purification methods were adjusted to suit the compounds. Such variances would be apparent to one skilled in the art.

TABLE 5

| Preparation | Chemical Name | Structure | Physical data |
|---|---|---|---|
| 17 | 6-Bromo-5-chloro-1- (tetrahydro-2H-pyran-2- yl)-1H-indazole | 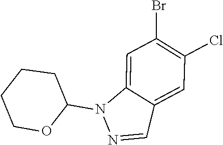 [SMILES: Clc1cc2cnn(C3CCCCO3)c2cc1Br] | ¹H-NMR (400 MHz, CDCl₃) δ 7.95 (m, 2H), 7.83 (s, 1H), 5.66 (m, 1H), 4.01 (m, 1H), 3.76 (m, 1H), 2.50 (m, 1H), 2.13 (m, 2H), 1.76 (m, 3H) |
| 18 | 5-Chloro-4-fluoro-1- (tetrahydro-2H-pyran-2- y1)-1H-pyrazolo[3,4- c]pyridine | 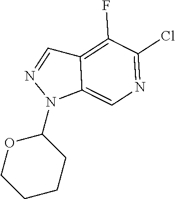 [SMILES: Fc1c(Cl)ncc2c1cnn2C1CCCCO1] | ES-MS m/z 255.9 (M + H) |
| 19ª | 5-Chloro-1-(tetrahydro- 2H-pyran-2-y1)-1H- pyrazolo[4,3-b]pyridine- 6-carbonitrile | 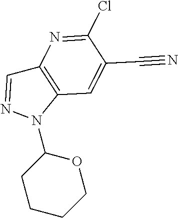 [SMILES: N#Cc1cc2c(cnn2C2CCCCO2)nc1Cl] | ES-MS m/z 263.1 (M + H) |

^{a}Stirred at 35° C.

### Preparation 20

### 6-Bromo-1-(4-methoxybenzyl)-1H-pyrazolo[4,3-b]pyridine

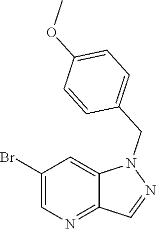 [SMILES: COc1ccc(Cn2ncc3ncc(Br)cc32)cc1]

Sodium hydride (3.966 g, 60 wt %, 99.14 mmol) was added to solution of 6-bromo-1H-pyrazolo[4,3-b]pyridine (12.27 g, 61.96 mmol) in DMF (206 mL) at 0° C. The reaction was stirred for 20 min, then 1-chloromethyl-4-methoxy-benzene (10.1 mL, 74.4 mmol) was added, and the mixture was allowed to stir overnight at RT. The reaction was quenched with sat. aq. NH₄Cl at 0° C., then diluted with water/sat. NH₄Cl and Et₂O. The aqueous layer was extracted twice with Et₂O. The combined organics were extracted with water, saturated aqueous NaCl, dried with MgSO₄, filtered, and concentrated. The residue was purified on silica gel, eluting with 5-30% EtOAc in cyclohexane to obtain the title compound (11.8 g, 60%) as a white solid. ES-MS m/z (⁷⁹B/⁸¹Br) 318.0, 320.0 (M+H).

### Preparation 21

### 1-(4-Methoxybenzyl)-1H-pyrazolo[4,3-b]pyridine-6-carbonitrile

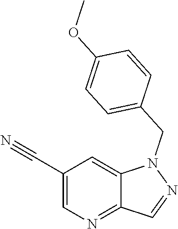 [SMILES: COc1ccc(Cn2ncc3ncc(C#N)cc32)cc1]

Pd(PPh₃)₄ (2.9 g, 2.5 mmol) was added to a degassed solution of zinc cyanide (6.504 g, 55.39 mmol) and 6-bromo-1-(4-methoxybenzyl)-1H-pyrazolo[4,3-b]pyridine (11.8 g, 36.9 mmol) in DMF (148 mL). The reaction was purged with N₂ and heated to 120° C. for 2 h. The reaction was diluted with EtOAc, filtered through diatomaceous earth and concentrated. The residue was purified on silica gel, eluting with 10-100% EtOAc in cyclohexane to obtain the title compound (9.0 g, 92%) as a white solid. ES-MS m/z 265.0 (M+H).

The compounds in the following table were prepared in similar manner as described in Preparation 21 from the appropriate aryl halide. Reactants can be added in different orders or in differing equivalency, and purification methods were adjusted to suit the compounds. Such variances would be apparent to one skilled in the art.

TABLE 6

| Preparation | Chemical Name | Structure | Physical data |
|---|---|---|---|
| 22ª | 5-Amino-2-chloro-6- (methylamino) nicotinonitrile | 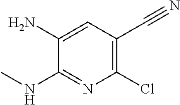 | ES-MS m/z 183.2 (M + H) |
| 23 | 5-Chloro-1-(tetrahydro- 2H-pyran-2-y1)-1H- indazole-6-carbonitrile | 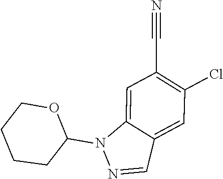 [SMILES: N#Cc1cc2c(cnn2C2CCCCO2)cc1Cl] | ¹H-NMR (400 MHz, DMSO-d₆) δ 8.60 (s, 1 H), 8.26 (s, 1H), 8.17 (s, 1H), 5.94 (d, J = 8.8 Hz, 1H), 3.88 (d, J = 11.6 Hz, 1H), 3.77 (m, 1H), 2.36 (m, 1H), 2.00 (m, 2H), 1.72 (m, 1H), 1.58 (m, 2H) |

^{a}Synthesized from the aryl iodide

### Preparation 24

### 5-Chloro-3-methyl-2-oxo-2,3-dihydro-1H-imidazo[4,5-b]pyridine-6-carbonitrile

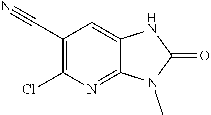

To a mixture of 5-amino-2-chloro-6-(methylamino) nicotinonitrile (220 mg, 0.964 mmol) in DMA (5 mL) was added N,N′-carbonyldiimidazole (797 mg, 4.82 mmol). The reaction mixture was stirred at 80° C. for 3 days under N₂, then filtered and concentrated. The filtrate was purified by reverse phase HPLC using a gradient of 18 to 58% ACN (with NH₄OAc as buffer) followed by recrystallization from EtOH (10 mL) to afford the title compound (40 mg, 19%) as a yellow solid. ES-MS m/z 207.0 (M−H).

### Preparation 25

### 6-Fluoro-1-methyl-1H-pyrazolo[4,3-b]pyridine

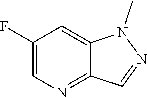

A mixture of 6-fluoro-1H-pyrazolo[4,3-b]pyridine (4.0 g, 29 mmol) and cesium carbonate (11.3 g, 34.7 mmol) was purged with N₂. DMF (130 mL) and methyl iodide (2.4 mL, 37 mmol) were added, and the resulting mixture was stirred at RT overnight. The reaction mixture was poured into water (100 mL) and EtOAc (50 mL). The layers were separated, and the aqueous layer was extracted with EtOAc (2×50 mL). The organic layers were combined, washed with water (twice) then sat. aq. NaCl, dried over Na₂SO₄, filtered and concentrated under reduced pressure. The residue was purified on silica gel, eluting with 0-90% EtOAc in cyclohexane, to obtain the first eluting isomer as the title compound as a white solid (1.78 g, 40%). ES-MS m/z 152.0 (M+H)

### Preparation 26

### 5-Fluoro-1-methyl-1H-pyrazolo[3,4-b]pyridine

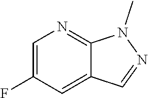

To a mixture of 5-fluoro-1H-pyrazolo[3,4-b]pyridine (1.8 g, 13.13 mmol) in DMSO (100 mL) was added NaHMDS (1.0 M in THF, 19.7 mL, 19.7 mmol) and iodomethane (2.46 mL, 39.3 mmol). The reaction was allowed to stir at RT for 2 h. The reaction was quenched with water and extracted with EtOAc (×3). The organic layers were combined, washed with water (×3), dried with Na₂SO₄, filtered and concentrated under reduced pressure. The residue was purified on silica gel, eluting with 0-40% EtOAc in cyclohexane to give the title compound as a white solid (1.3 g, 66%). 1H-NMR (400 MHz, DMSO-d₆) δ 8.64-8.58 (m, 1H), 8.19-8.11 (, 2H), 4.07 (s, 3H).

### Preparation 27

### 6-Fluoro-1-methyl-1H-pyrazolo[4,3-b]pyridine 4-oxide

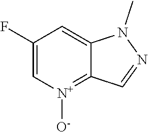

A mixture of 6-fluoro-1-methyl-1H-pyrazolo[4,3-b]pyridine (1.22 g, 8.07 mmol) in DCM (33 mL) under N₂ atmosphere was treated with 3-chloroperoxybenzoic acid (3.78 g, 16.4 mmol). The resulting mixture was stirred at RT for 4 h. The reaction was cooled to 0° C. to produce a white precipitate which was removed by vacuum filtration. The filter cake was washed with cold DCM. The filtrate was washed with 1 M NaOH (50 mL). The aqueous layer was extracted with DCM until no product remained in the aqueous layer. The combined organic layer was dried with Na₂SO₄, filtered and concentrated under reduced pressure to afford the title compound as a white solid (1.23 g, 86%). ES/MS m/z 167.8 (M+H).

The compounds in the following table were prepared in similar manner as described in Preparation 27 from the appropriate aza-indazole. Reactants can be added in different orders or in differing equivalency, and purification methods were adjusted to suit the compounds. Such variances would be apparent to one skilled in the art.

TABLE 7

| Preparation | Chemical Name | Structure | ES-MS m/z |
|---|---|---|---|
| 28ª | 6-Cyano-1-(4- methoxybenzyl)-1H- pyrazolo[4,3-b]pyridine 4-oxide | 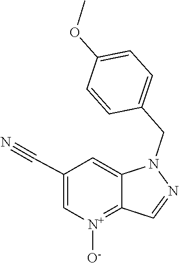 [SMILES: COc1ccc(Cn2ncc3c2cc(C#N)c[n+]3[O-])cc1] | 281.0 (M + H) |
| 29^{b} | 5-Fluoro-1-methyl-1H- pyrazolo[3,4-b]pyridine 7-oxide | 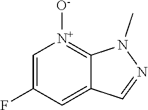 | 168.0 (M + H) |

^{a}The product was purified by silica gel 10 — 100% EtOAc/DCM.
bThe reaction solvent used was EtOAc, heated to 45° C. for 18 h.

### Preparation 30

### 5-Chloro-6-fluoro-1-methyl-1H-pyrazolo[4,3-b]pyridine

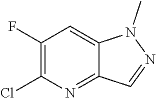

A mixture of 6-fluoro-1-methyl-1H-pyrazolo[4,3-b]pyridine 4-oxide (500.0 mg, 3.0 mmol) in DCM (54 mL) was purged with N₂. The flask was cooled to 0° C. and TEA (1.05 mL, 7.5 mmol) and oxalyl chloride (0.66 mL, 7.5 mmol) were added sequentially. The reaction was allowed to stir while warming to RT for 2 h. The reaction mixture was diluted with DCM and poured into sat. aq. NaHCO₃ (50 mL). The layers were separated, and the organic layer was washed with sat. aq. NaHCO₃ (50 mL) and sat. aq. NaCl (50 mL), dried over Na₂SO₄, filtered and concentrated under reduced pressure. The residue was purified on silica gel, eluting with 0-100% EtOAc in cyclohexane, to obtain the title compound as a white solid (490 mg, 86%). ES-MS m/z 186.2 (M+H).

The compounds in the following table were prepared in similar manner as described in Preparation 30 from the appropriate N-oxide. Reactants can be added in different orders or in differing equivalency, and purification methods were adjusted to suit the compounds. Such variances would be apparent to one skilled in the art.

TABLE 8

| Preparation | Chemical Name | Structure | Physical data |
|---|---|---|---|
| 31ª | 5-Chloro-1-(4- methoxybenzyl)-1H- pyrazolo[4,3-b]pyridine- 6-carbonitrile | 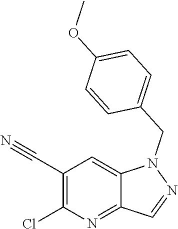 [SMILES: COc1ccc(Cn2ncc3nc(Cl)c(C#N)cc32)cc1] | ES-MS m/z 299.0 (M + H) |
| 32 | 6-Chloro-5-fluoro-1- methyl-1H- pyrazolo[3,4-b]pyridine | 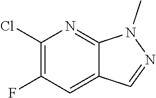 | ¹H-NMR (400 MHz, CDCl₃) δ 7.99 (s, 1H), 7.80 (d, J = 7.7 Hz, 1H), 4.15 (s, 3H) |

^{a}The compound was recrystallized from EtOAc and cyclohexanes.

### Preparation 33

### 2-Bromo-5-(2-methyl-2H-pyrazolo[3,4-b]pyridin-5-yl)benzonitrile

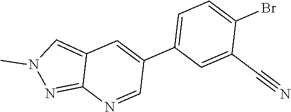 [SMILES: Cn1cc2cc(-c3ccc(Br)c(C#N)c3)cnc2n1]

To mixture of (2-methyl-2H-pyrazolo[3,4-b]pyridin-5-yl)boronic acid (2.87 g, 16.2 mmol), 2-bromo-5-iodobenzonitrile (5.00 g, 16.2 mmol), cesium carbonate (21.2 g, 65.0 mmol) and Pd(dppf)Cl₂ (1.07 g, 1.46 mmol) under N₂ atmosphere was added 1,4-dioxane/water (3:1) (81.2 mL). A long needle was inserted below the mixture surface and N₂ gas was bubbled through the mixture for 5 min. The mixture was heated to 55° C. for 1 h, allowed to cool to ambient temperature and water (700 mL) was added while stirred. The mixture was filtered via vacuum filtration to yield a brown solid cake which was dissolved into DCM and dried over MgSO₄, filtered and concentrated under vacuum. The residue was purified by silica gel, eluting with 0-5% MeOH/DCM. The resulting residue was crystallized from cyclohexane/EtOAc to provide the title compound (3.1 g, 61%) as a light yellow. ES-MS m/z (⁷⁹Br/⁸¹Br) 212.8/314.8

### Preparation 34

### 5-Chloro-1-methyl-1H-indazole-6-carbonitrile

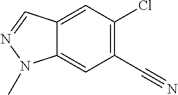

### and 5-chloro-2-methyl-2H-indazole-6-carbonitrile

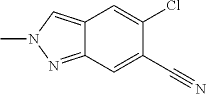

To a mixture of 5-chloro-1H-indazole-6-carbonitrile (1.0 g, 5.6 mmol) and K₂CO₃ (2.33 g, 16.9 mmol) in DMF (28 mL) was added iodomethane (1.0 mL, 16.9 mmol). The reaction was allowed to stir at RT for 19 h. The reaction was quenched with water (60 mL) and extracted with EtOAc (3×60 mL). The organic layers were combined, washed with water (2×), washed with sat. aq. NaCl, dried with Na₂SO₄, filtered and concentrated under reduced pressure. The residue was purified on silica gel, eluting with 20-40% EtOAc in cyclohexane to give 5-chloro-1-methyl-1H-indazole-6-carbonitrile (480 mg, 45%) and 5-chloro-2-methyl-2H-indazole-6-carbonitrile (210 mg, 20%) as white solids.

The compounds in the following table were prepared in similar manner as described in Preparation 34 from the appropriate NH-indazole. Reactants can be added in different orders or in differing equivalency, and purification methods were adjusted to suit the compounds. Two isomers were isolated that were separated using common purification methods. Such variances would be apparent to one skilled in the art.

TABLE 9

| Preparation | Chemical Name | Structure | ES-MS m/z |
|---|---|---|---|
| 35ª | 6-Chloro-1-methyl-1H- indazole-5-carbonitrile | 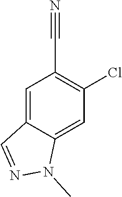 | 191.9 (M + H) |
| 36ª | 6-Chloro-2-methyl-2H- indazole-5-carbonitrile | 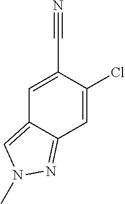 | 192.0 (M + H) |
| 37ª | 5-Chloro-1-methyl-1H- pyrazolo[4,3-b]pyridine- 6-carbonitrile | 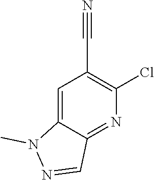 | 193.1 (M + H) |
| 38ª | 5-Chloro-2-methyl-2H- pyrazolo[4,3-b]pyridine- 6-carbonitrile | 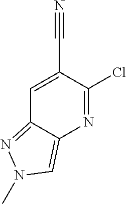 | 193.1 (M + H) |
| 39 | 5,6-Dibromo-1-methyl- 1H- benzo[d][1,2,3 ]triazole | 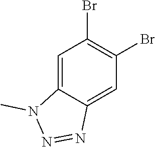 | 291.9 (M + H) |

^{a}1.2 equiv of NaH was used instead of K₂CO₃

### Preparation 40

### 5-Bromo-1-methyl-1H-benzo[d][1,2,3]triazole-6-carbonitrile

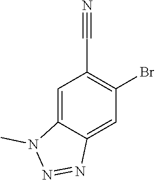

### and 6-bromo-1-methyl-1H-benzo[d][1,2,3]triazole-5-carbonitrile

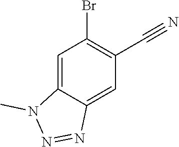

A mixture of 5,6-dibromo-1-methyl-1H-benzo[d][1,2,3]triazole (300 mg, 1.01 mmol) and cuprous cyanide (136 mg, 46.6 μL, 1.52 mmol) in NMP (5 mL) was heated at 150° C. for 9 h under microwave irradiation in a sealed tube, then combined with a second reaction mixture (170 μmol scale). NH₃·H₂O (0.3 mL) was added to the reaction mixture and the reaction mixture was diluted with H₂O (20 mL) and extracted with EtOAc (3×30 mL). The combined organic layers were washed with sat. aq. NaCl (3×30 mL), dried over anhydrous Na₂SO₄, filtered and concentrated under reduced pressure. The residue was purified by flash silica gel, eluting with 0-28% EtOAc/petroleum to provide the mixture of the regio isomers. The mixture was separated by SFC [column: Daicel Chiralpak® IG (250 mm×30 mm, 10 μm); mobile phase: 30% EtOH in CO_{2+0.1}% NH₄OH); flowrate (mL/min): 80] to obtain 5-bromo-1-methyl-1H-benzo[d][1,2,3]triazole-6-carbonitrile (40 mg, 15%), ES-MS m/z 237.0 (M+H); and the second eluting isomer 6-bromo-1-methyl-1H-benzo[d][1,2,3]triazole-5-carbonitrile (30 mg, 12%), ES-MS m/z 237.0 (M+H).

### Preparation 41

### tert-Butyl (1-(5-bromo-3-(trifluoromethyl)-1H-pyrazol-1-yl)-2-methylpropan-2-yl)carbamate

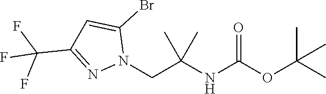 [SMILES: CC(C)(Cn1nc(C(F)(F)F)cc1Br)NC(=O)OC(C)(C)C]

A mixture of 3-bromo-5-(trifluoromethyl)-1H-pyrazole (4.11 g, 19.1 mmol), tert-butyl 4,4-dimethyl-1,2,3-oxathiazolidine-3-carboxylate 2,2-dioxide (4.00 g, 15.9 mmol) and Cs₂CO₃ (10.4 g, 31.8 mmol) was purged with N₂. DMA (32 mL) was added, and the resulting mixture was stirred at 95° C. for 6 h. The reaction mixture was cooled and poured into water (350 mL) and EtOAc (75 mL). The layers were separated, and the aq. layer was extracted with EtOAc (4× 40 mL). The organic layers were combined, washed with water (40 mL) then sat. aq. NaCl (50 mL), dried over Na₂SO₄, filtered, and concentrated under reduced pressure. The residue was purified on silica gel, eluting with 0-10% MTBE in heptane, to obtain the second eluting isomer as the title compound (4.11 g, 67%). ES-MS m/z (⁷⁹Br/⁸¹Br) 330.0/332.0 (M-tBu+H).

### Preparation 42

### tert-Butyl (2-methyl-1-(5-(1-methyl-6-oxo-1,6-dihydropyridin-3-yl)-3-(trifluoromethyl)-1H-pyrazol-1-yl)propan-2-yl)carbamate

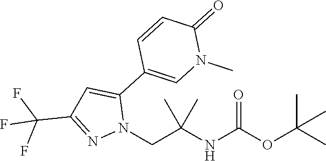 [SMILES: Cn1cc(-c2cc(C(F)(F)F)nn2CC(C)(C)NC(=O)OC(C)(C)C)ccc1=O]

A mixture of 1-methyl-5-(4,4,5,5-tetramethyl-1,3,2-dioxaborolan-2-yl)pyridin-2(1H)-one (3.04 g, 12.9 mmol), tert-butyl (1-(5-bromo-3-(trifluoromethyl)-1H-pyrazol-1-yl)-2-methylpropan-2-yl)carbamate (5.00 g, 12.9 mmol), Cs₂CO₃ (8.44 g, 25.9 mmol) and Pd(dppf)Cl₂ (947 mg, 1.29 mmol), 1,4-dioxane (78.5 mL) and water (7.85 mL) was sparged with N₂ for 5 min. The resulting mixture was stirred at 60° C. for 1 h. Another portion of 1-methyl-5-(4,4,5,5-tetramethyl-1,3,2-dioxaborolan-2-yl)pyridin-2(1H)-one (1.3 g, 5.53 mmol) was added, and the mixture was stirred at 80° C. overnight. The reaction mixture was cooled to RT, diluted with DCM, and washed with water. The organic phase was dried over MgSO₄ and concentrated under reduced pressure. The residue was purified on silica gel, eluting with 30-100% EtOAc in cyclohexane. Crystallization from 20% EtOAc: 80% cyclohexane, followed by vacuum filtration gave the title compound (2.77 g, 52%) as a white solid. ES-MS m/z 359.0 (M-tBu+H).

### Preparation 43

### 1-(5-Bromo-3-(trifluoromethyl)-1H-pyrazol-1-yl)-2-methylpropan-2-amine hydrochloride

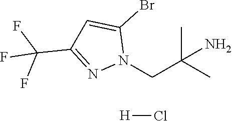

A solution of tert-butyl (1-(5-bromo-3-(trifluoromethyl)-1H-pyrazol-1-yl)-2-methylpropan-2-yl)carbamate (5.00 g, 12.9 mmol) in MeOH (12.9 mL) was treated with 4M HCl in dioxane (12.9 mL, 51.8 mmol). After stirring for 2 h at RT, the mixture was concentrated to obtain the title compound (4.145 g, 99%) as a white solid. ES-MS (m/z, ⁷⁹Br/⁸¹Br) 286.0/288.0 (M+H).

The compound in the following table was prepared in similar manner as described in Preparation 43 from the appropriate tert-butyl carbamate. Different reaction times (2-43 h), eq of HCl 4-18 eq), and co-solvents (MeOH, EtOH, EtOAc, dioxane) can be used. Such variances would be apparent to one skilled in the art.

TABLE 10

| Preparation | Chemical Name | Structure | ES-MS m/z |
|---|---|---|---|
| 44 | 5-(1-(2-Amino-2- methylpropyl)-3- (trifluoromethyl)-1H- pyrazol-5-y1)-1- methylpyridin-2(1H)- one hydrochloride | 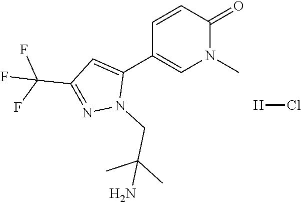 | 315.0 (M + H) |

### Preparation 45

### N-(1-Cyano-1-(4-fluorophenyl)-2-methylpropan-2-yl)-2-methylpropane-2-sulfinamide

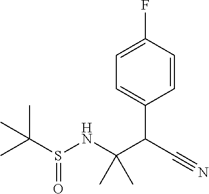 [SMILES: CC(C)(NS(=O)C(C)(C)C)C(C#N)c1ccc(F)cc1]

To a solution of lithium diisopropylamide (5.7 g, 53 mmol) was added 2-(4-fluorophenyl)acetonitrile (3.0 g, 22 mmol) in THF (30 mL) at −70° C. The mixture was stirred for 30 min under N₂ atmosphere. Then, chlorotitanium triisopropoxide (14 mL, 55 mmol) was added and the reaction was stirred at −70° C. for 20 min. 2-Methyl-N-(propan-2-ylidene)propane-2-sulfinamide (8.0 g, 44 mmol) in THF (30 mL) was then added and the mixture was stirred at −70° C. for 1 h. The reaction mixture was poured into water (20 mL) and extracted with EtOAc (2×20 mL). The organic layers were combined and washed with water (2×10 mL), dried over Na₂SO₄, filtered and concentrated under reduced pressure. The residue was purified on silica, eluting with 0-40% EtOAc in PE to give the title compound as a colorless oil (2.65 g, 36%). ES-MS m/z 296.9 (M+H).

### Preparation 46

### 3-((tert-Butylsulfinyl)amino)-2-(4-fluorophenyl)-N′-hydroxy-3-methylbutanimidamide

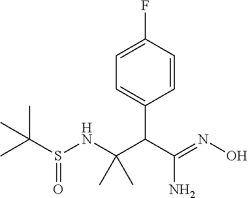 [SMILES: CC(C)(NS(=O)C(C)(C)C)C(/C(N)=N/O)c1ccc(F)cc1]

To a mixture of N-(1-cyano-1-(4-fluorophenyl)-2-methylpropan-2-yl)-2-methylpropane-2-sulfinamide (2.65 g, 8.05 mmol) and TEA (3.2 mL, 23 mmol) in EtOH (20 mL) was added hydroxylamine hydrochloride (1.35 g, 19.4 mmol). The reaction was heated to 80° C. for 16 h under N₂ atmosphere. The reaction was cooled to RT and concentrated under reduced pressure. The residue was purified on silica, eluting with 0-5% MeOH in DCM to give the title compound as a colorless oil (550 mg, 17.3%). ES-MS m/z 330.2 (M+H).

### Preparation 47

### N-(1-(4-Fluorophenyl)-2-methyl-1-(5-oxo-4,5-dihydro-1,2,4-thiadiazol-3-yl)propan-2-yl)-2-methylpropane-2-sulfinamide

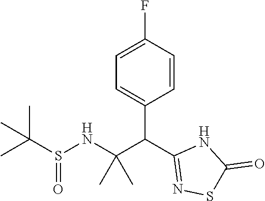 [SMILES: CC(C)(NS(=O)C(C)(C)C)C(c1ccc(F)cc1)c1nsc(=O)[nH]1]

To a mixture of 3-((tert-butylsulfinyl)amino)-2-(4-fluorophenyl)-N′-hydroxy-3-methylbutanimidamide (550 mg, 1.39 mmol) in THF (8 mL) was added di-1H-imidazol-1-ylmethanethione (0.27 mL, 1.96 mmol). The reaction was stirred at 15° C. for 4 h. The reaction mixture was added to water (20 mL) and extracted with EtOAc (2×20 mL). The organic layers were combined and washed with sat. aq. NaCl (2× 10 mL), dried over Na₂SO₄, filtered and concentrated under reduced pressure. To the residue was added THF (8 mL) and boron trifluoride etherate (1 mL, 8 mmol) and the reaction was stirred at 15° C. for 16 h. The reaction mixture was concentrated under reduced pressure. The residue was purified on silica, eluting with 0-4% MeOH in DCM to give the title compound as a yellow solid (150 mg, 26%). ES-MS m/z 372.2 (M+H).

### Preparation 48

### 3-((tert-Butylsulfinyl)amino)-3-methyl-2-(3-(trifluoromethyl)phenyl)butanoic acid

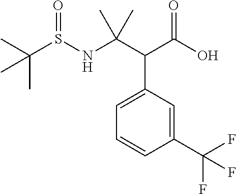 [SMILES: CC(C)(NS(=O)C(C)(C)C)C(C(=O)O)c1cccc(C(F)(F)F)c1]

To a solution of 2-(3-(trifluoromethyl)phenyl)acetic acid (5.80 g, 28.4 mmol) in THF (40 mL) was added lithium diisopropylamide solution (35.5 mL, 2 molar, 271.0 mmol) at −70° C. and the reaction was stirred at −70° C. for 30 min. Then, chlorotitanium triisopropoxide (31.1 g, 30.5 mL, 119 mmol) in THF (30 mL) was added at −70° C. and stirred for 30 min. After 30 min, 2-methyl-N-(propan-2-ylidene)propane-2-sulfinamide (9.67 g, 90 wt %, 54.0 mmol) in THF (20 mL) was added. The mixture was stirred at −70° C. for 1 h under N₂. The reaction was quenched by the addition of water (50 mL) and the solids were removed by filtration, washing with EtOAc (3×30 mL). The filtrate was washed with EtOAc (3×50 mL). The aqueous layer was adjusted to pH 6 by the addition of 1 M HCl then, the aqueous layer was extracted with EtOAc (3×50 mL). The organic layers were combined, dried with Na₂SO₄, filtered and concentrated under reduced pressure. The residue was purified on silica, eluting with 0-9% MeOH in DCM to give the title compound as a yellow oil (1.2 g, 80% purity, 9.2%) and a yellow oil (1.8 g, 50% purity, 8.7%). ES-MS m/z 366.0 (M+H).

### Preparation 49

### Methyl 3-((tert-butylsulfinyl)amino)-3-methyl-2-(3-(trifluoromethyl)phenyl)butanoate

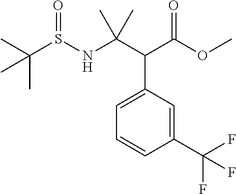 [SMILES: COC(=O)C(c1cccc(C(F)(F)F)c1)C(C)(C)NS(=O)C(C)(C)C]

To a solution of 3-((tert-butylsulfinyl)amino)-3-methyl-2-(3-(trifluoromethyl)phenyl)butanoic acid (3.7 g, 50 wt %, 5.1 mmol) in MeOH (20 mL) and DCM (20 mL) was added (trimethylsilyl)diazomethane solution (12 g, 15 mL, 2 molar, 30 mmol) at 25° C. The mixture was stirred at 25° C. for 12 h under N₂. The mixture was quenched by 0.2 mL HOAc, then concentrated under reduced pressure to give a crude, then combined with a second reaction crude (2.6 mmol scale). The combined crude was purified by silica gel, eluting with 0˜6% DCM:MeOH gradient to give the compound methyl 3-((tert-butylsulfinyl)amino)-3-methyl-2-(3-(trifluoromethyl)phenyl)butanoate (1.87 g, 86% purity, 62% yield) as a yellow oil. ES-MS m/z 380.4 (M+H).

### Preparation 50

### Ethyl 2-(4-(trifluoromethyl)-1H-pyrazol-1-yl)acetate

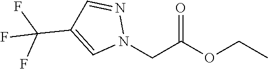

To a mixture of 4-(trifluoromethyl)-1H-pyrazole (5.00 g, 36.7 mmol) and potassium carbonate (10.2 g, 73.5 mmol) in DMF (50 mL) was added ethyl 2-bromoacetate (5.28 mL 47.8 mmol). The mixture was stirred at 60° C. for 12 h. The reaction mixture was poured into water (50 mL) and extracted EtOAc (50 mL×3). The combined organic layers were washed with sat. aq. NaCl (50 mL), dried over Na₂SO₄ and concentrated. The residue was purified by flash silica gel, eluting with 0% DCM/MeOH gradient to give the title compound (6.8 g, 79% yield) as a colorless oil. ES-MS m/z M+H=223.2.

### Preparation 51

### Ethyl 3-((tert-butylsulfinyl)amino)-3-methyl-2-(4-(trifluoromethyl)-1H-pyrazol-1-yl)butanoate

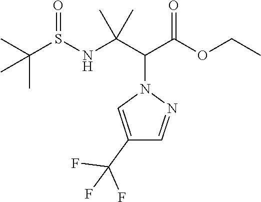 [SMILES: CCOC(=O)C(n1cc(C(F)(F)F)cn1)C(C)(C)NS(=O)C(C)(C)C]

To a solution of ethyl 2-(4-(trifluoromethyl)-1H-pyrazol-1-yl)acetate (5.00 g, 21.38 mmol) in THF (50 mL) was added lithium diisopropylamide solution (25.66 mL, 2 molar, 51.31 mmol) at −70° C. and stirred for 30 min. Then chlorotitanium triisopropoxide (15.25 g, 55.59 mmol) in THF (25 mL) was added at −70° C. and stirred for 30 min, then 2-methyl-N-(propan-2-ylidene)propane-2-sulfinamide (prepared essentially as described in WO2022170198 A1, 7.258 g, 42.76 mmol) in THF (25 mL) was added. The mixture was stirred at −70° C. for 1 h under N₂. To the reaction mixture was added water (100 mL) and the solids were filtered, and the filter cake was washed with EtOAc (100 mL×3). The filtrate was diluted with water (100 mL) and extracted with EtOAc (100 mL×3). The combined organic layers were washed with sat. aq. NaCl (100 mL×2), dried over Na₂SO₄, filtered and concentrated under reduced pressure. The residue was purified by reverse phase HPLC using a gradient of 25 to 65% ACN with NH₄OAc buffer. The eluent was concentrated to remove organic solvent and the residual aqueous solution was lyophilized. The compound the title compound (3.3 g, 31% yield, 77% purity) was obtained as a yellow oil. ES-MS m/z 384.3 (M+H).

The compound in the following table was prepared in similar manner as described in Preparation 51 from methyl 2-(4-fluorophenyl)acetate. Different reaction times (1-16 h), equiv. of chlorotitanium triisopropoxide (2.6-4.2 equiv.), and equiv. of lithium diisopropylamide (2.4-3.3 equiv.) can be used. Such variances would be apparent to one skilled in the art.

TABLE 11

| Preparation | Chemical Name | Structure | ES-MS m/z |
|---|---|---|---|
| 52 | Methyl 3-((tert- butylsulfinyl)amino)-2- (4-fluorophenyl)-3- methylbutanoate | 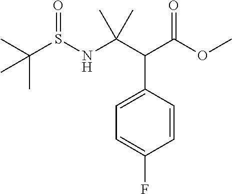 [SMILES: COC(=O)C(c1ccc(F)cc1)C(C)(C)NS(=O)C(C)(C)C] | 330.1 (M + H) |

### Preparation 53

### 3-(2-Amino-1-(4-fluorophenyl)-2-methylpropyl)-1,2,4-thiadiazol-5 (4H)-one hydrochloride

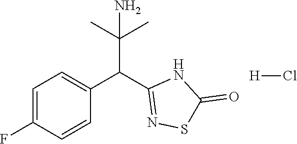

To a solution of N-(1-(4-fluorophenyl)-2-methyl-1-(5-oxo-4,5-dihydro-1,2,4-thiadiazol-3-yl)propan-2-yl)-2-methylpropane-2-sulfinamide (150 mg, 0.37 mmol) in 1,4-dioxane (1 mL) was added hydrogen chloride (2.0 mL, 2.0 M in 1,4-dioxane, 4.0 mmol) at 15° C. The mixture was stirred at 15° C. for 3 h. The reaction mixture was concentrated directly under reduced pressure to obtain the title compound (130 mg, 93% yield, 80% purity) as a yellow solid. ES-MS m/z 269.1 (M+H).

The compounds in the following table were prepared in similar manner as described in Preparation 53 from the appropriate (tert-butylsulfinyl)amines. Different reaction times (1-2 h), equiv of HCl (4-10 equiv), temperature (0-25° C.) and co-solvents (MeOH, EtOH, DCM, dioxane) can be used. Such variances would be apparent to one skilled in the art.

TABLE 12

| | | | ES-MS |
|---|---|---|---|
| Preparation | Chemical Name | Structure | m/z |
| 54 | Methyl 3-amino-3-methyl- 2-(3- (trifluoromethyl)phenyl) butanoate hydrochloride | 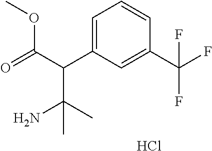 [SMILES: COC(=O)C(c1cccc(C(F)(F)F)c1)C(C)(C)N ; Cl] | 276.0 (M + H) |
| 55 | Ethyl 3-amino-3-methyl-2- (4-(trifluoromethyl)-1H- pyrazol-1-yl)butanoate hydrochloride | 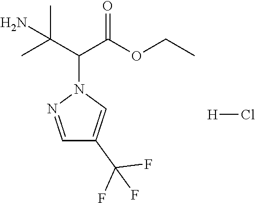 [SMILES: CCOC(=O)C(n1cc(C(F)(F)F)cn1)C(C)(C)N ; Cl] | 280.1 (M + H) |
| 56ª | Methyl 3-amino-2-(4- fluorophenyl)-3- methylbutanoate | 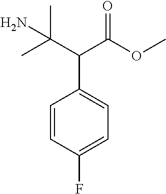 [SMILES: COC(=O)C(c1ccc(F)cc1)C(C)(C)N] | 225.9 (M + H) |

^{a}The compound isolated as the free base after extraction from aq. NaHCO₃ with EtOAc.

### Preparation 57

### (1-(2-((tert-Butoxycarbonyl)amino)-2-methylpropyl)-3-(trifluoromethyl)-1H-pyrazol-5-yl)boronic acid

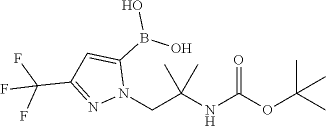 [SMILES: CC(C)(Cn1nc(C(F)(F)F)cc1B(O)O)NC(=O)OC(C)(C)C]

In a glovebox, a solution of tert-butyl (1-(5-bromo-3-(trifluoromethyl)-1H-pyrazol-1-yl)-2-methylpropan-2-yl)carbamate (1.0 g, 2.5 mmol) in MeOH (30 mL) was treated with tetrahydroxydiboron (1.1 g, 13 mmol), a solution of XPhos Pd G3 (CAS #1445085-55-1, 0.22 g, 0.25 mmol) in DCE (3 mL), and KOAc (1.0 g, 10 mmol). The resulting mixture was stirred at 40° C. for 16 h under N₂. The reaction was repeated on the same scale, and the reaction solutions were combined, adjusted pH to 6 with FA and filtered. The filtrate was concentrated. The residue was purified on silica gel, eluting with 0-4% (0.1% FA/MeOH) in DCM, to obtain the title compound (1.45 g, 80%) as a brown oil. ES-MS m/z 352.3 (M+H).

### Preparation 58

### 3-Bromo-5-methoxy-1,2,4-thiadiazole

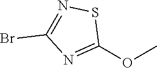

To a solution of 3-bromo-5-chloro-1,2,4-thiadiazole (25.00 g, 122.8 mmol) in MeOH (80 mL) was added sodium methoxide (13.97 g, 245.7 mmol). The mixture was stirred at 25° C. under N₂ for 16 h. An additional portion of sodium methoxide (6.985 g, 122.8 mmol) was added and stirring continued at 25° C. under N₂ for 16 h. The mixture was concentrated under reduced pressure. The residue was purified on silica gel, eluting with 0-5% EtOAc in PE, to obtain the title compound (9.7 g, 36% yield, 90% purity) as a white solid. ES-MS m/z 196.7 (M+H).

### Preparation 59

### tert-Butyl (1-(5-(5-methoxy-1,3,4-thiadiazol-2-yl)-3-(trifluoromethyl)-1H-pyrazol-1-yl)-2-methylpropan-2-yl)carbamate

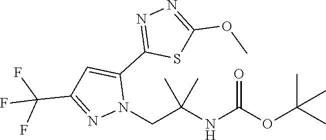 [SMILES: COc1nnc(-c2cc(C(F)(F)F)nn2CC(C)(C)NC(=O)OC(C)(C)C)s1]

A solution of 2-bromo-5-methoxy-1,3,4-thiadiazole (526 mg, 2.56 mmol), (1-(2-((tert-butoxycarbonyl)amino)-2-methylpropyl)-3-(trifluoromethyl)-1H-pyrazol-5-yl)boronic acid (1.00 g, 2.56 mmol, ˜90% purity) and Na₂CO₃ (543 mg, 5.13 mmol) in 1,4-dioxane (10 mL), EtOH (10 mL) and water (1 mL) was treated with XPhos Pd G3 (CAS #1445085-55-1, 325 mg, 384 μmol). After stirring at 90° C. for 2 h under N₂, the reaction mixture was cooled and diluted with water (20 mL). The aq. layer was extracted with EtOAc (2×30 mL). The organic layers were combined, washed with sat. aq. NaCl (3×15 mL), dried over Na₂SO₄, filtered, and concentrated under reduced pressure. The residue was purified on silica gel, eluting with 0-40% EtO Ac in PE to obtain the title compound (170 mg, 14%) as a yellow solid. ES-MS m/z 422.0 (M+H).

The compound in the following table was prepared in similar manner as described in Preparation 59 using the appropriate boronic acid and heteroaryl bromide. Different catalysts and cosolvents may be used. Such variances would be apparent to one skilled in the art.

TABLE 13

| | | | ES-MS |
|---|---|---|---|
| Preparation | Chemical Name | Structure | m/z |
| 60ª | tert-Butyl (1-(5-(5- methoxy-1,2,4- thiadiazol-3-yl)-3- (trifluoromethyl)-1H- pyrazol-1-yl)-2- methylpropan-2- y1)carbamate | 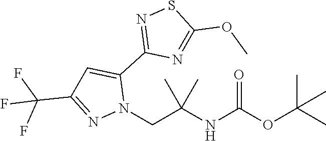 [SMILES: COc1nc(-c2cc(C(F)(F)F)nn2CC(C)(C)NC(=O)OC(C)(C)C)ns1] | 444.0 (M + Na) |

^{a}Reagents used: Pd(dppf)Cl₂, Na₂CO₃, 1,4-dioxane/water; the product was purified on silica gel eluting with 0-3% MeOH/DCM.

### Preparation 61

### 5-(1-(2-Amino-2-methylpropyl)-3-(trifluoromethyl)-1H-pyrazol-5-yl)-1,3,4-thiadiazol-2-ol hydrochloride

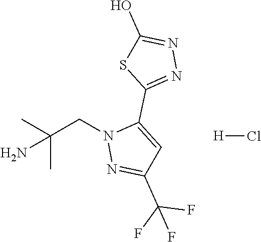 [SMILES: CC(C)(N)Cn1nc(C(F)(F)F)cc1-c1nnc(O)s1 ; Cl]

A solution of tert-butyl (1-(5-(5-methoxy-1,3,4-thiadiazol-2-yl)-3-(trifluoromethyl)-1H-pyrazol-1-yl)-2-methylpropan-2-yl)carbamate (70 mg, 0.14 mmol) in 1,4-dioxane (1 mL) was treated with 2 M HCl in 1,4-dioxane (400 mL, 800 mmol). After stirring for 16 h at RT, the mixture was treated with additional 2 M HCl in 1,4-dioxane (300 mL, 600 mmol). After stirring for an additional 16 h at RT the mixture was concentrated to obtain the title compound (60 mg, 68% purity, 82% yield) as a white solid. ES-MS m/z 307.9 (M+H).

The compound in the following table was prepared in similar manner as described in Preparation 61 using tert-butyl (1-(5-(5-methoxy-1,2,4-thiadiazol-3-yl)-3-(trifluoromethyl)-1H-pyrazol-1-yl)-2-methylpropan-2-yl)carbamate and 2 M HCl in 1,4-dioxane.

TABLE 14

| | | | ES-MS |
|---|---|---|---|
| Preparation | Chemical Name | Structure | m/z |
| 62 | 3-(1-(2-Amino-2- methylpropyl)-3- (trifluoromethyl)-1H-pyrazol- 5-y1)-1,2,4-thiadiazol-5-ol hydrochloride | 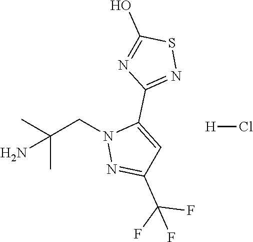 [SMILES: CC(C)(N)Cn1nc(C(F)(F)F)cc1-c1nsc(O)n1 ; Cl] | 307.9 (M + H) |

### Preparation 63

### 1-(5-(5-Methoxy-1,2,4-thiadiazol-3-yl)-3-(trifluoromethyl)-1H-pyrazol-1-yl)-2-methylpropan-2-amine 2,2,2-trifluoroacetate

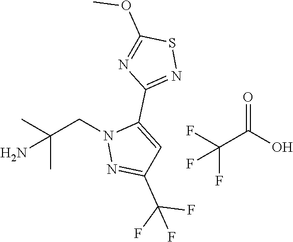

To a solution of tert-butyl (1-(5-(5-methoxy-1,2,4-thiadiazol-3-yl)-3-(trifluoromethyl)-1H-pyrazol-1-yl)-2-methylpropan-2-yl)carbamate (1.5 g, 80% purity, 2.8 mmol) in DCM (30 mL) was added TFA (3.3 mL, 43 mmol) at 15° C. Then the mixture was stirred at 15° C. for 1 h to provide a solution of the title compound in DCM. This solution was used directly in the following step assuming quantitative yield. ES-MS m/z 322.2 (M+H).

### Preparation 64

### 1-((5-Fluoropyridin-2-yl)methyl)-2,4,6-triphenylpyridin-1-ium tetrafluoroborate

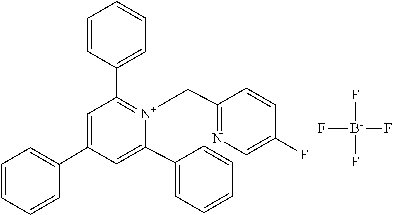 [SMILES: Fc1ccc(C[n+]2c(-c3ccccc3)cc(-c3ccccc3)cc2-c2ccccc2)nc1 ; F[B-](F)(F)F]

(5-Fluoropyridin-2-yl)methanamine (4.99 g, 39.5 mmol) was added to a mixture of 2,4,6-triphenylpyrylium tetrafluoroborate (14.5 g, 36.6 mmol) in DCM (100 mL) at RT, and the resulting mixture was stirred overnight before concentrating to a foam. The foam was triturated with Et₂O, and the solvent was decanted off (repeated). The remaining residue was dried overnight under vacuum to obtain the title compound as a yellow foam, which was used directly in the next step assuming quantitative yield. ES-MS m/z 417.0 (M+).

### Preparation 65

### 5-Fluoro-2-(2-methyl-2-nitropropyl)pyridine

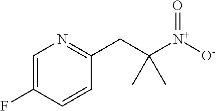

2-Nitropropane (12.9 mL, 144 mmol) was added over 10 min to an ice-water bath cooled solution of sodium methoxide (5.4 molar solution in MeOH, 22.2 mL, 120 mmol) in MeOH (240 mL). The resulting mixture was stirred for 30 min at RT and then concentrated to give a white solid that was dried under vacuum overnight. To the white solid was added 1-((5-fluoropyridin-2-yl)methyl)-2,4,6-triphenylpyridin-1-ium tetrafluoroborate (19 g, 38 mmol) and DMSO (50 mL) in a 250 mL, three-neck flask equipped with a mechanical stirrer under N₂. The resulting mixture was stirred at 70° C. for 2 h. The mixture was cooled to RT and then diluted with Et₂O (200 mL) to give a precipitate. The solids were removed by filtration through diatomaceous earth, rinsing with Et₂O. The filtrate (800 mL) was washed with water (200 mL). The layers were separated, and the aq. layer was extracted with Et₂O (3×50 mL). The organic layers were combined, washed with sat. aq. NaCl, dried over MgSO₄, filtered and concentrated. The residue was purified on silica gel, eluting with 5-100% EtOAc in DCM, to obtain the title compound (5.63 g, 75%) as a yellow oil. ES-MS m/z 198.8 (M+H).

### Preparation 66

### 1-(5-Fluoropyridin-2-yl)-2-methylpropan-2-amine hydrochloride

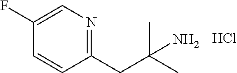

A solution of 5-fluoro-2-(2-methyl-2-nitropropyl)pyridine (4.62 g, 23.3 mmol) in MeOH (100 mL) was added to a mixture of Raney® Nickel (19.28 g, 328.5 mmol) in MeOH (100 mL) in a pressure vessel under a stream of N₂. The vessel was sealed, purged with N₂ 5 times, purged with H₂ 5 times, and pressurized with H₂ to 60 psi. After shaking at RT for 5 h, the suspension was filtered over diatomaceous earth, and the solids were rinsed with MeOH to give a clear, light-yellow filtrate, which was combined with the filtrate from a second reaction ran in similar manner on 996 mg of 5-fluoro-2-(2-methyl-2-nitropropyl)pyridine. The combined filtrate was concentrated. The residue was dissolved in Et₂O (200 mL) and filtered through fluted filter paper to remove solids. The filtrate was concentrated to give 1-(5-fluoropyridin-2-yl)-2-methylpropan-2-amine (4.19 g) as a light orange oil. The oil was dissolved in ether (200 mL) and treated with 4M HCl in dioxane (7.0 mL, 28 mmol) to give a precipitate. The solvent was removed under reduced pressure to obtain a quantitative yield of the title compound (6.55 g) as a yellow solid. ES/MS m/z 169.0 (M+H).

### Preparation 67

### tert-Butyl 2-(2-((tert-butoxycarbonyl)amino)-2-methylpropyl)hydrazine-1-carboxylate

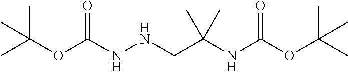 [SMILES: CC(C)(CNNC(=O)OC(C)(C)C)NC(=O)OC(C)(C)C]

To a solution of tert-butyl (2-methyl-1-oxopropan-2-yl)carbamate (9.0 g, 48 mmol) in MeOH (100 mL) was added tert-butyl hydrazinecarboxylate (6.4 g, 48 mmol) and HOAc (8.3 mL, 140 mmol). The mixture was stirred at RT for 2 h, then NaBH₃CN (4.5 g, 72 mmol) was added. After stirring at 25° C. for 16 h, the reaction mixture was concentrated under reduced pressure and diluted with water (100 mL). Na₂CO₃ was added to adjusted to pH-8, and the mixture was extracted with EtOAc (2×100 mL). The organic layers were combined, washed with sat. aq. NaCl (100 mL), dried over Na₂SO₄, filtered, and concentrated under reduced pressure. The residue was purified on silica gel, eluting with 0-10% EtOAc in PE, to obtain the title compound (13.2 g, 86%) as a white solid. ES-MS m/z 304.2 (M+H).

### Preparation 68

### 1-Hydrazineyl-2-methylpropan-2-amine trihydrochloride

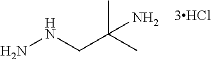

To a solution of tert-butyl 2-(2-((tert-butoxycarbonyl)amino)-2-methylpropyl)hydrazine-1-carboxylate (15.2 g, 47.6 mmol) in MeOH (100 mL) was added hydrogen chloride (100 mL, 4 M in MeOH, 400 mmol). The mixture was stirred at 25° C. for 16 h, then concentrated under reduced pressure to provide the title compound (8.8 g, 83%) as a white solid. 1H-NMR (400 MHz, CD₃OD) δ 3.10 (s, 2H), 1.38 (s, 6H).

### Preparation 69

### 1-(5-Cyclopropyl-1H-pyrazol-1-yl)-2-methylpropan-2-amine

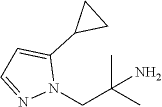

To a mixture of 1-hydrazineyl-2-methylpropan-2-amine trihydrochloride (2.5 g, 11 mmol) and 1-cyclopropyl-3-(dimethylamino) prop-2-en-1-one (1.6 g, 11 mmol) in EtOH (10 mL) was added TEA (1.6 mL, 11 mmol). The reaction mixture was stirred at 80° C. for 16 h. The reaction mixture was then concentrated under reduced pressure to provide the title compound (3.2 g, 96% yield, 60% purity) as a yellow solid. ES-MS m/z 180.3 (M+H).

### Preparation 70

### Methyl (R)-2-((tert-butoxycarbonyl)amino)-3-(1-methyl-1H-pyrazol-4-yl)propanoate

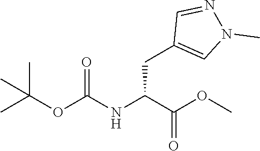 [SMILES: COC(=O)[C@@H](Cc1cnn(C)c1)NC(=O)OC(C)(C)C]

A mixture of 4-iodo-1-methyl-1H-pyrazole (20.0 g, 96.2 mmol), pyridine-2,6-bis(carboximidamide)dihydrochloride (5.05 g, 19.2 mmol), nickel (II) chloride dimethoxyethane adduct (CAS #29046-78-4, 4.23 g, 19.2 mmol), methyl(S)-2-((tert-butoxycarbonyl)amino)-3-iodopropanoate (47.5 g, 144 mmol) and zinc (8.46 g, 129 mmol) in DMA (300 mL) was purged with N₂ and stirred at 40° C. for 16 h under N₂. The reaction mixture was quenched slowly with sat. aq. NH₄Cl solution (300 mL) at 0° C. then filtered. The filtrate was diluted with water (200 mL) and extracted with EtOAc (3× 400 mL). The organic layers were combined, washed with water (2×300 mL), then sat. aq. NaCl (300 mL), dried over anhydrous Na₂SO₄, filtered, and concentrated under reduced pressure. The residue was purified on silica gel, eluting with 0-33% EtO Ac in PE to obtain the title compound (12.16 g, 42%) as a colorless oil. ES-MS m/z 284.1 (M+H). >99% ee, [Chiral SFC—column: Chiralcel® OJ-3, 4.6×150 mm, 3 μm; column temperature: 35° C.; mobile phase: gradient of 5-50% EtOH (with 0.05% DMEA) in CO₂; flow rate: 3 mL/min].

### Preparation 71

### Methyl (R)-2-((tert-butoxycarbonyl)amino)-3-(1-(difluoromethyl)-1H-pyrazol-4-yl)propanoate

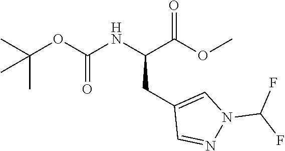 [SMILES: COC(=O)[C@@H](Cc1cnn(C(F)F)c1)NC(=O)OC(C)(C)C]

To a solution of 1-difluoromethyl-4-iodo-1H-pyrazole (15.0 g, 59.6 mmol) in DMA (50 mL) under N₂, was added pyridine-2,6-bis(carboximidamide)dihydrochloride (2.83 g, 12.0 mmol), methyl(S)-2-((tert-butoxycarbonyl)amino)-3-iodopropanoate (29.5 g, 89.7 mmol), zinc (7.79 g, 119 mmol) and nickel (II) chloride dimethoxyethane adduct (CAS #29046-78-4, 2.60 g, 11.8 mmol). The resulting mixture was stirred at 60° C. (internally) for 12 h. The reaction mixture was quenched with sat. aq. NH₄Cl solution (200 mL) then filtered over diatomaceous earth, rinsing with EtOAc (2×50 mL). The filtrate was diluted with water (200 mL) and extracted with EtOAc (3×100 mL). The organic layers were combined, washed sat. aq. NaCl (100 mL), dried over Na₂SO₄, filtered, and concentrated under reduced pressure. The residue was purified on silica, eluting with 0-10% EtOAc in PE, to obtain the title compound (13.0 g, 67%) as a colorless oil. ES-MS m/z 264.1 (M-tBu+H). >99% ee, [Chiral SFC-Column: Chiralpak® AD-3, 4.6×150 mm, 3 μm; column temperature: 35° C.; mobile phase: gradient from 10-50% EtOH (with 0.2% 7M NH₃ in MeOH) in CO₂; flow rate: 2.5 mL/min].

### Preparation 72

### (R)-2-((tert-Butoxycarbonyl)amino)-3-(1-(difluoromethyl)-1H-pyrazol-4-yl)propanoic acid

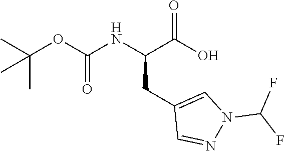 [SMILES: CC(C)(C)OC(=O)N[C@H](Cc1cnn(C(F)F)c1)C(=O)O]

A solution of methyl (R)-2-((tert-butoxycarbonyl)amino)-3-(1-(difluoromethyl)-1H-pyrazol-4-yl)propanoate (12.0 g, 36.8 mmol) in THF (120 mL) was treated with 1M aq. LiOH·H₂O (110 mL, 110 mmol) and stirred at 25° C. for 1 h under N₂. The mixture was extracted with DCM (3×30 mL). The aq. phase was adjusted to pH 2-3 with 1N aq. HCl and the resulting white solid was collected by filtration, suspended in PE (100 mL), and stirred for 12 h. Filtration and drying under vacuum afforded the title compound (10.5 g, 94%) as a white solid. ES-MS m/z 250.1 (M-tBu+H). >99% ee [SFC—column: Chiralpak® AD-3, 4.6×150 mm, 3 μm; column temperature: 35° C.; mobile phase: 15% IPA (with 0.5% IPAm): 85% CO₂; flow rate: 2.5 mL/min].

The compound in the following table was prepared in similar manner as described in Preparation 72. Alternatively, after acidification of the reaction mixture, the products can be extracted with EtOAc. These compounds could also be isolated as their corresponding Lithium carboxylate salts by concentrating the basic reaction mixture, which would be apparent to one skilled in the art.

TABLE 15

| Preparation | Chemical Name | Structure | ES-MS m/z |
|---|---|---|---|
| 73 | (R)-2-((tert- Butoxycarbonyl) amino)-3-(1-methyl- 1H-pyrazol-4- yl)propanoic acid | 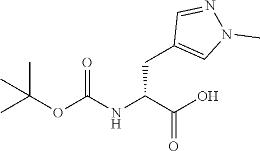 [SMILES: Cn1cc(C[C@@H](NC(=O)OC(C)(C)C)C(=O)O)cn1] | 270.2 (M + H) |

### Preparation 74

### tert-Butyl (R)-(1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate

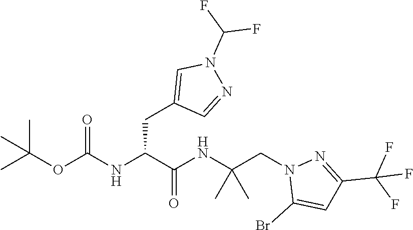 [SMILES: CC(C)(Cn1nc(C(F)(F)F)cc1Br)NC(=O)[C@@H](Cc1cnn(C(F)F)c1)NC(=O)OC(C)(C)C]

A stirring mixture of (R)-2-((tert-butoxycarbonyl)amino)-3-(1-(difluoromethyl)-1H-pyrazol-4-yl)propanoic acid (8.3 g, 27 mmol), 1-(5-bromo-3-(trifluoromethyl)-1H-pyrazol-1-yl)-2-methylpropan-2-amine hydrochloride (8.4 g, 26 mmol), DIEA (16 mL, 91 mmol), and DMF (50 mL) was cooled at 0° C. and treated with HATU (12 g, 31 mmol) portion-wise. After 4 h at RT, the reaction mixture was partitioned between EtOAc (400 mL) and water (400 mL). The aq. layer was extracted with EtOAc (300 mL). The organic layers were combined, washed with water (500 mL), then sat. aq. NaCl (500 mL), dried over Na₂SO₄, filtered, and concentrated under reduced pressure. The residue was purified on silica gel, eluting with 50-65% MTBE in heptane, to obtain the title compound (14.4 g, 96%) as a white foam. ES-MS m/z (⁷⁹Br/⁸¹Br) 517.2/519.2 (M-tBu+H).

The compounds in the following table were prepared in similar manner as described in Preparation 74 using the appropriate carboxylic acid and the appropriate amine. Reactants can be added in different orders or in differing equivalency. DMA and DMSO are suitable replacements for DMF. Reaction times (30 min to overnight) and temperatures (0° C. to RT) may vary, and differing methods can be used to work up or purify the compounds (normal phase, or high, neutral, or low pH reversed phase). Such variances would be apparent to one skilled in the art.

TABLE 16

| Preparation | Chemical Name | Structure | ES-MS m/z |
|---|---|---|---|
| 75 | (tert-butyl (R)-(1-((2- methyl-1-(5-(1-methyl- 6-oxo-1,6- dihydropyridin-3-yl)-3- (trifluoromethyl)-1H- pyrazol-1-yl)propan-2- yl)amino)-3-(1-methyl- 1H-pyrazol-4-yl)-1- oxopropan-2- yl)carbamate | 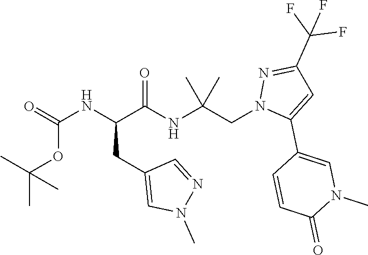 [SMILES: Cn1cc(C[C@@H](NC(=O)OC(C)(C)C)C(=O)NC(C)(C)Cn2nc(C(F)(F)F)cc2-c2ccc(=O)n(C)c2)cn1] | 566.4 (M + H) |
| 76 | tert-butyl (R)-(1-((1-(5- fluoropyridin-2-yl)-2- methylpropan-2- yl)amino)-3-(1-methyl- 1H-pyrazol-4-yl)-1- oxopropan-2- yl)carbamate | 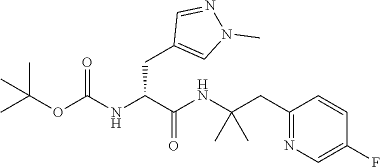 [SMILES: Cn1cc(C[C@@H](NC(=O)OC(C)(C)C)C(=O)NC(C)(C)Cc2ccc(F)cn2)cn1] | 420.1 (M + H) |
| 77 | tert-butyl (R)-(1-((1-(5- cyclopropyl-1H-pyrazol- 1-yl)-2-methylpropan-2- yl)amino)-3-(1-methyl- 1H-pyrazol-4-yl)-1- oxopropan-2- yl)carbamate | 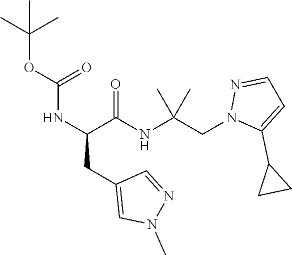 [SMILES: Cn1cc(C[C@@H](NC(=O)OC(C)(C)C)C(=O)NC(C)(C)Cn2nccc2C2CC2)cn1] | 431.3 (M + H) |
| 78 | methyl 3-((R)-2-((tert- butoxycarbonyl)amino)- 3-(1-methyl-1H-pyrazol- 4-yl)propanamido)-3- methyl-2-(3- (trifluoromethyl)phenyl) butanoate | 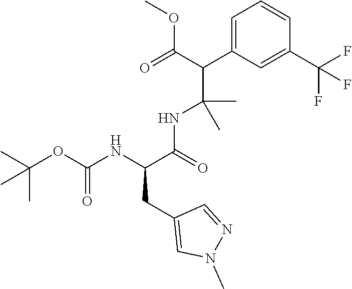 [SMILES: COC(=O)C(c1cccc(C(F)(F)F)c1)C(C)(C)NC(=O)[C@@H](Cc1cnn(C)c1)NC(=O)OC(C)(C)C] | 527.1 (M + H) |
| 79 | methyl 3-((R)-2-((tert- butoxycarbonyl)amino)- 3-(1-methyl-1H-pyrazol- 4-yl)propanamido)-2-(4- fluorophenyl)-3- methylbutanoate | 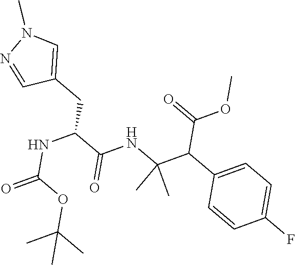 [SMILES: COC(=O)C(c1ccc(F)cc1)C(C)(C)NC(=O)[C@@H](Cc1cnn(C)c1)NC(=O)OC(C)(C)C] | 477.1 (M + H) |
| 80 | ethyl 3-((R)-2-((tert- butoxycarbonyl)amino)- 3-(1-methyl-1H-pyrazol- 4-yl)propanamido)-3- methyl-2-(4- (trifluoromethyl)-1H- pyrazol-1-yl)butanoate | 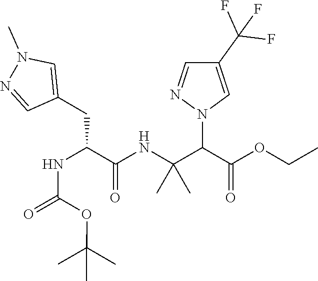 [SMILES: CCOC(=O)C(n1cc(C(F)(F)F)cn1)C(C)(C)NC(=O)[C@@H](Cc1cnn(C)c1)NC(=O)OC(C)(C)C] | 531.6 (M + H) |
| 81 | tert-butyl (R)-(1-((1-(5- (5-methoxy-1,2,4- thiadiazol-3-yl)-3- (trifluoromethyl)-1H- pyrazol-1-yl)-2- methylpropan-2- yl)amino)-3-(1-methyl- 1H-pyrazol-4-yl)-1- oxopropan-2- yl)carbamate | 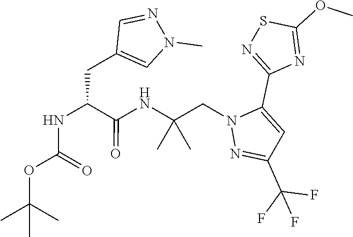 [SMILES: COc1nc(-c2cc(C(F)(F)F)nn2CC(C)(C)NC(=O)[C@@H](Cc2cnn(C)c2)NC(=O)OC(C)(C)C)ns1] | 573.4 (M + H) |
| 82 | tert-Butyl (R)-(1-((1-(5- bromo-3- (trifluoromethyl)-1H- pyrazol-1-yl)-2- methylpropan-2- yl)amino)-3-(1-methyl- 1H-pyrazol-4-yl)-1- oxopropan-2- yl)carbamate | 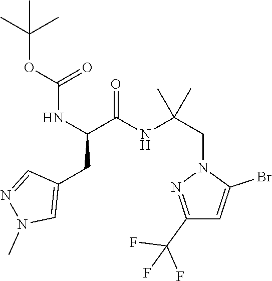 [SMILES: Cn1cc(C[C@@H](NC(=O)OC(C)(C)C)C(=O)NC(C)(C)Cn2nc(C(F)(F)F)cc2Br)cn1] | (⁷⁹B/⁸¹Br) 481.0/483.0 (M − tBu + H) |

### Preparation 83

### tert-Butyl (R)-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)carbamate

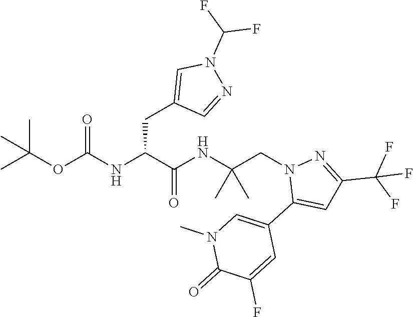 [SMILES: Cn1cc(-c2cc(C(F)(F)F)nn2CC(C)(C)NC(=O)[C@@H](Cc2cnn(C(F)F)c2)NC(=O)OC(C)(C)C)cc(F)c1=O]

To a mixture of tert-butyl (R)-(1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate (2.05 g, 3.58 mmol), 3-fluoro-1-methyl-5-(4,4,5,5-tetramethyl-1,3,2-dioxaborolan-2-yl)pyridin-2(1H)-one (900 mg, 3.56 mmol), K₂CO₃ (1.47 g, 10.6 mmol) and Pd(dppf)Cl₂·CH₂Cl₂ (581 mg, 711 μmol) under N₂ was added N₂ sparged 4:1 dioxane/water (20 mL). The resulting mixture was stirred at 90° C. for 3.5 h, then cooled to RT and filtered. The filtrate was concentrated under reduced pressure and the residue was purified on silica gel, eluting with 0-100% EtOAc/cyclohexane, to obtain the title compound (1.25 g, 57%) as a yellow solid. ES-MS m/z 520.2 (M-BOC+H), 642.2 (M+Na).

### Preparation 84

### tert-Butyl (R)-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)carbamate

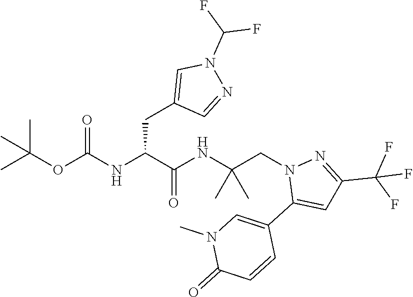 [SMILES: Cn1cc(-c2cc(C(F)(F)F)nn2CC(C)(C)NC(=O)[C@@H](Cc2cnn(C(F)F)c2)NC(=O)OC(C)(C)C)ccc1=O]

To a 1 L RBF equipped with a stir bar was added 1-methyl-5-(4,4,5,5-tetramethyl-1,3,2-dioxaborolan-2-yl)pyridin-2(1H)-one (30.8 g 131 mmol), tert-butyl (R)-(1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate (50.0 g, 87.2 mmol), Pd(dppf)Cl₂ (5.5 g, 6.7 mmol), and K₂CO₃ (36.2 g, 262 mmol) followed by 1,4-dioxane (279 mL) and water (69.8 mL). The mixture was vacuum degassed and purged with N₂ (3×), heated to 90° C. for 1 h. The mixture was diluted with DCM (500 mL) washed with water (1 L) The organic phase was dried over magnesium sulfate and concentrated under reduced pressure to yield a dark oil. The material was purified by flash silica gel column eluting with 0-5% MeOH in EtOAc to provide the title compound (42 g, 80%) as a light tan foam solid. ES-MS m/z 602.4 (M+H)

The compounds in the following table were prepared in similar manner as described in Preparations 83 and 84 using the appropriate aryl bromide and the appropriate boron ester or boronic acid. Reactants can be added in different orders or in differing equivalency, reaction times (5-32 h) and temperatures (90-100° C.) may vary, and differing methods can be used to work up or purify the compounds, which would be apparent to one skilled in the art.

TABLE 17

| | | | ES-MS |
|---|---|---|---|
| Preparation | Chemical Name | Structure | m/z |
| 85 | tert-butyl ((2R)-3-(1- (difluoromethyl)-1H- pyrazol-4-yl)-1-((2-methyl- 1-(5-(2-(tetrahydro-2H- pyran-2-yl)-2H-1,2,3- triazol-4-yl)-3- (trifluoromethyl)-1H- pyrazol-1-yl)propan-2- yl)amino)-1-oxopropan-2- yl)carbamate | 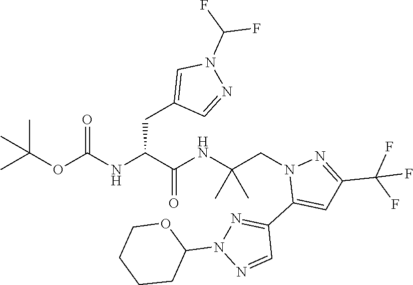 [SMILES: CC(C)(Cn1nc(C(F)(F)F)cc1-c1cnn(C2CCCCO2)n1)NC(=O)[C@@H](Cc1cnn(C(F)F)c1)NC(=O)OC(C)(C)C] | 646.2 (M + H) |
| 86 | tert-butyl ((2R)-1-((2- methyl-1-(5-(2-(tetrahydro- 2H-pyran-2-yl)-2H-1,2,3- triazol-4-yl)-3- (trifluoromethyl)-1H- pyrazol-1-yl)propan-2- yl)amino)-3-(1-methyl-1H- pyrazol-4-yl)-1-oxopropan- 2-yl)carbamate | 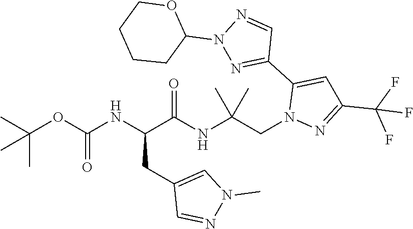 [SMILES: Cn1cc(C[C@@H](NC(=O)OC(C)(C)C)C(=O)NC(C)(C)Cn2nc(C(F)(F)F)cc2-c2cnn(C3CCCCO3)n2)cn1] | 632.4 (M + Na) |

### Preparation 87

### (R)-2-Amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide hydrochloride

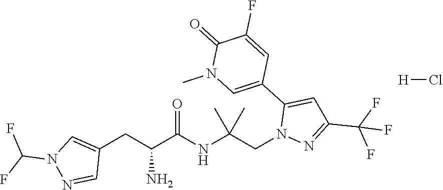

A solution of tert-butyl (R)-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)carbamate (1.25 g, 2.02 mmol) in MeOH (10 mL) was added 4 M HCl in 1,4-dioxane (7.6 mL, 30 mmol). After stirring for 18 h at RT, the mixture was concentrated under a stream of N₂ to obtain the title compound (1.1 g, 98%) as a yellow solid. ES-MS (m/z) 520.0 (M+H).

### Preparation 88

### (R)-2-Amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide hydrochloride

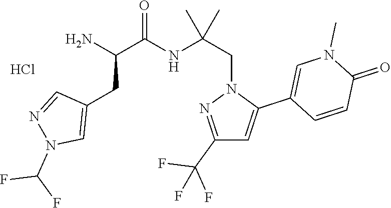 [SMILES: Cn1cc(-c2cc(C(F)(F)F)nn2CC(C)(C)NC(=O)[C@H](N)Cc2cnn(C(F)F)c2)ccc1=O ; Cl]

To a 1 L RBF with stir bar was added: tert-butyl (R)-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)carbamate (67.4 g, 112 mmol) and 1,4-dioxane (280 mL). The mixture was heated to 40° C. and monitored with an internal temperature probe. HCl (4M in 1,4-dioxane, 140 mL, 560 mmol) was added over 15 min and the mixture was stirred overnight. The mixture was cooled to ambient temperature and 100 mL of MeOH was added. The solution was concentrated under reduced pressure at 40° C. This residue was then dissolved in MeOH (100 mL) and DCM (200 mL) and concentrated to provide the title compound (68.3 g, quantitative yield) as a light tan foam solid. ES-MS (m/z) 502.2 (M+H).

The compounds in the following table were prepared in similar manner as described in Preparations 87 and 88 from the appropriate tert-butyl carbamate. Different reaction times (15 min-24 h), equivalents of HCl (3-25 eq), and co-solvents (such as EtOAc) can be used. Such variances would be apparent to one skilled in the art. Alternative conditions, TFA in DCM, provided the TFA salt. The deprotected amines were isolated as the corresponding salt.

TABLE 18

| | | | ES-MS |
|---|---|---|---|
| Preparation | Chemical Name | Structure | m/z |
| 89 | (R)-2-Amino-N-(2-methyl-1-(5-(1- methyl-6-oxo-1,6-dihydropyridin- 3-yl)-3-(trifluoromethyl)-1H- pyrazol-1-yl)propan-2-yl)-3-(1- methyl-1H-pyrazol-4- yl)propanamide hydrochloride | 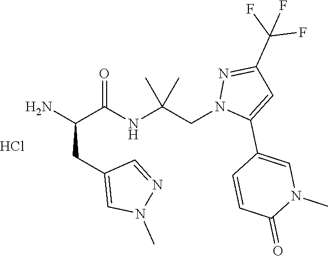 [SMILES: Cl ; Cn1cc(C[C@@H](N)C(=O)NC(C)(C)Cn2nc(C(F)(F)F)cc2-c2ccc(=O)n(C)c2)cn1] | 466.4 (M + H) |
| 90 | (R)-2-Amino-N-(1-(5- fluoropyridin-2-yl)-2- methylpropan-2-yl)-3-(1-methyl- 1H-pyrazol-4-yl)propanamide hydrochloride | 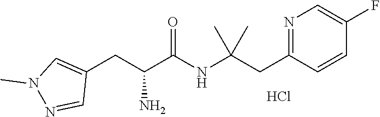 [SMILES: Cn1cc(C[C@@H](N)C(=O)NC(C)(C)Cc2ccc(F)cn2)cn1 ; Cl] | 320.2 (M + H) |
| 91 | (R)-2-Amino-N-(1-(5-cyclopropyl- 1H-pyrazol-1-yl)-2-methylpropan- 2-yl)-3-(1-methyl-1H-pyrazol-4- yl)propanamide hydrochloride | 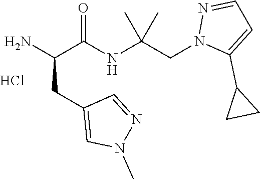 [SMILES: Cl ; Cn1cc(C[C@@H](N)C(=O)NC(C)(C)Cn2nccc2C2CC2)cn1] | 331.3 (M + H) |
| 92 | Methyl 3-((R)-2-amino-3-(1- methyl-1H-pyrazol-4- yl)propanamido)-3-methyl-2-(3- (trifluoromethyl)phenyl)butanoate hydrochloride | 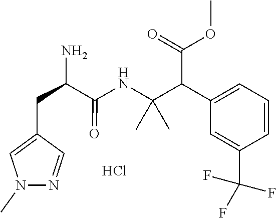 [SMILES: COC(=O)C(c1cccc(C(F)(F)F)c1)C(C)(C)NC(=O)[C@H](N)Cc1cnn(C)c1 ; Cl] | 427.2 (M + H) |
| 93 | Methyl 3-((R)-2-amino-3-(1- methyl-1H-pyrazol-4- yl)propanamido)-2-(4- fluorophenyl)-3-methylbutanoate hydrochloride | 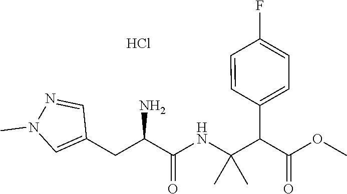 | 377.0 (M + H) |
| 94 | Ethyl 3-((R)-2-amino-3-(1-methyl- 1H-pyrazol-4-yl)propanamido)-3- methyl-2-(4-(trifluoromethyl)-1H- pyrazol-1-yl)butanoate hydrochloride | 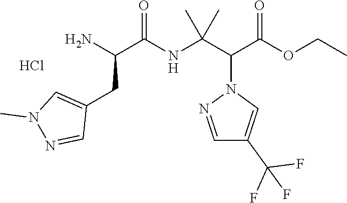 [SMILES: CCOC(=O)C(n1cc(C(F)(F)F)cn1)C(C)(C)NC(=O)[C@H](N)Cc1cnn(C)c1 ; Cl] | 431.4 (M + H) |
| 95^{a} | (R)-2-Amino-N-(1-(5-(5-methoxy- 1,2,4-thiadiazol-3-yl)-3- (trifluoromethyl)-1H-pyrazol-1- yl)-2-methylpropan-2-yl)-3-(1- methyl-1H-pyrazol-4- yl)propanamide 2,2,2- trifluoroacetate | 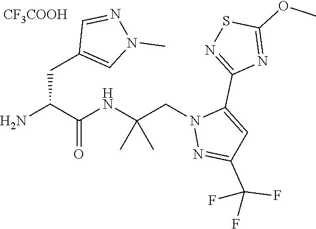 [SMILES: "CF3COOH" ; COc1nc(-c2cc(C(F)(F)F)nn2CC(C)(C)NC(=O)[C@H](N)Cc2cnn(C)c2)ns1] | 473.2 (M + H) |

^{a}The deprotection was performed with TFA in DCM

### Preparation 96

### (R)—N-(1-(5-(2H-1,2,3-Triazol-4-yl)-3-(trifluoromethyl)-1H-pyrazol-1-yl)-2-methylpropan-2-yl)-2-amino-3-(1-(difluoromethyl)-1H-pyrazol-4-yl)propanamide hydrochloride

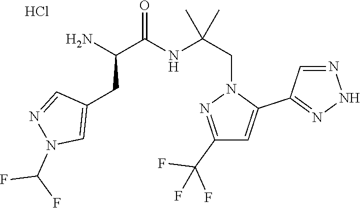 [SMILES: CC(C)(Cn1nc(C(F)(F)F)cc1-c1cn[nH]n1)NC(=O)[C@H](N)Cc1cnn(C(F)F)c1 ; Cl]

A solution of tert-butyl ((2R)-3-(1-(difluoromethyl)-1H-pyrazol-4-yl)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-1-oxopropan-2-yl)carbamate (150 mg, 204 μmol, ˜88% purity) in MeOH (3 mL) was treated with 2 M HCl in MeOH (2.0 mL, 4.0 mmol). The resulting mixture was stirred at 25° C. for 12 h then concentrated under reduced pressure to obtain quantitative yield of the title compound (140 mg, ˜70% purity) as a yellow solid. ES-MS m/z 462.2 (M+H).

### Preparation 97

### (2R)-2-Amino-N-(2-methyl-1-(5-(2-(tetrahydro-2H-pyran-2-yl)-2H-1,2,3-triazol-4-yl)-3-(trifluoromethyl)-1H-pyrazol-1-yl)propan-2-yl)-3-(1-methyl-1H-pyrazol-4-yl)propanamide hydrochloride and (R)—N-(1-(5-(2H-1,2,3-triazol-4-yl)-3-(trifluoromethyl)-1H-pyrazol-1-yl)-2-methylpropan-2-yl)-2-amino-3-(1-methyl-1H-pyrazol-4-yl)propanamide hydrochloride (mixture)

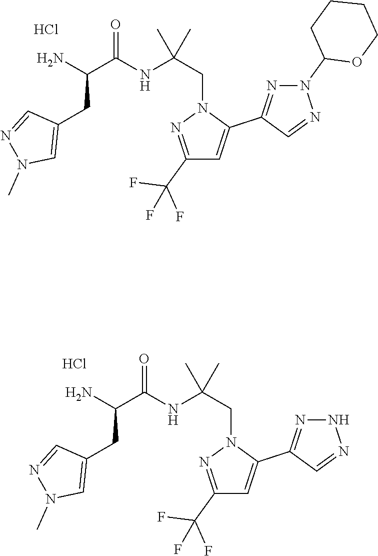 [SMILES: Cn1cc(C[C@@H](N)C(=O)NC(C)(C)Cn2nc(C(F)(F)F)cc2-c2cnn(C3CCCCO3)n2)cn1 ; Cl ; Cn1cc(C[C@@H](N)C(=O)NC(C)(C)Cn2nc(C(F)(F)F)cc2-c2cn[nH]n2)cn1 ; Cl]

To a solution of tert-butyl ((2R)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-3-(1-methyl-1H-pyrazol-4-yl)-1-oxopropan-2-yl)carbamate (4.14 g, 90% purity, 6.11 mmol) in MeOH (15 mL) was added 2 M HCl in 1,4-dioxane (20 mL, 2 molar, 40 mmol). The mixture was stirred at 30° C. for 1 h, then concentrated directly to provide the title compounds as a 3:1 mixture of THP-protected to THP-deprotected products (3.6 g, quantitative yield assumed) as a yellow solid. For (2R)-2-amino-N-(2-methyl-1-(5-(2-(tetrahydro-2H-pyran-2-yl)-2H-1,2,3-triazol-4-yl)-3-(trifluoromethyl)-1H-pyrazol-1-yl)propan-2-yl)-3-(1-methyl-1H-pyrazol-4-yl)propanamide hydrochloride: ES-MS m/z 510.3 (M+H).

For (R)—N-(1-(5-(2H-1,2,3-triazol-4-yl)-3-(trifluoromethyl)-1H-pyrazol-1-yl)-2-methylpropan-2-yl)-2-amino-3-(1-methyl-1H-pyrazol-4-yl)propanamide hydrochloride: ES-MS m/z 426.2 (M+H).

### Preparation 98

### Methyl (Z)-2-(((benzyloxy)carbonyl)amino)-3-(1-methyl-1H-pyrazol-4-yl)acrylate

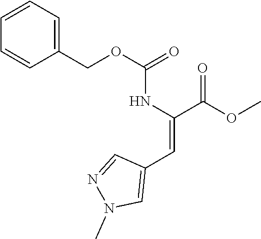 [SMILES: COC(=O)/C(=C/c1cnn(C)c1)NC(=O)OCc1ccccc1]

To a cooled mixture of 1-methyl-1H-pyrazole-4-carbaldehyde (300 g, 2.72 mol) and methyl 2-(((benzyloxy)carbonyl)amino)-2-(dimethoxyphosphoryl)acetate (966 g, 2.92 mol) in DCM (1.5 L) was added DBU (435 mL, 2.89 mol) in one portion at 0-5° C. under N₂. The mixture was warmed to 15-20° C. and stirred for 2 h. The reaction mixture was diluted with water (2 L) and extracted with DCM (2× 0.5 L). The organic layers were combined, dried over Na₂SO₄, filtered, and concentrated under reduced pressure. The residue was triturated by stirring with MTBE (3 L) for 1 h, before filtering to collect the solids. The reaction was repeated ten times. Combination of the collected material from all eleven reactions gave the title compound (7.00 kg, 74%) as a yellow solid. ES-MS m/z 316.1 (M+H).

### Preparation 99

### Methyl 2-(((benzyloxy)carbonyl)amino)-3-(1-methyl-1H-pyrazol-4-yl)propanoate

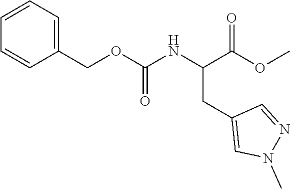 [SMILES: COC(=O)C(Cc1cnn(C)c1)NC(=O)OCc1ccccc1]

To a solution of methyl (Z)-2-(((benzyloxy)carbonyl)amino)-3-(1-methyl-1H-pyrazol-4-yl)acrylate (700 g, 2.22 mol) in MeOH (7 L) was added diacetato[(R)-(+)-2,2′-bis(diphenylphosphino)-1,1′-binaphthyl]ruthenium (II) (Ru(OAc) 2 [(R)-binap], CAS #325146-81-4, 46.8 g, 55.5 mmol) and HOAc (127 mL, 2.22 mol) under Ar. The resulting mixture was degassed under vacuum and purged with H₂ several times. After stirring under H₂ (435 psi) at 60° C. for 16 h, the reaction mixture was concentrated under reduced pressure. The reaction was repeated nine times on the same scale. The residues from all ten reactions were combined to obtain the title compound (6.80 kg, 97%) as a yellow oil which is enantiomerically enriched with the (R)-enantiomer (˜3:1). ES-MS m/z 318.1 (M+H).

### Preparation 100

### (R)-2-(((Benzyloxy)carbonyl)amino)-3-(1-methyl-1H-pyrazol-4-yl)propanoic acid

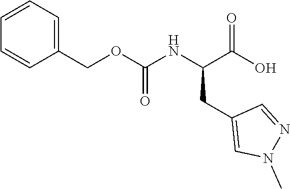 [SMILES: Cn1cc(C[C@@H](NC(=O)OCc2ccccc2)C(=O)O)cn1]

A 0° C. solution of methyl 2-(((benzyloxy)carbonyl)amino)-3-(1-methyl-1H-pyrazol-4-yl)propanoate, enantiomerically enriched with the (R)-enantiomer (3.40 kg, 10.7 mol) in MeOH (10.2 L) was treated with a mixture of LiOH. H₂O (1.80 kg, 42.9 mol) and H₂O (13.6 L). After stirring at 20° C. for 16 h, the mixture was partially concentrated under reduced pressure, adjusted to pH=3-4 with 1 M aq. H₂SO₄, and filtered to collect the solids. The reaction was repeated on the same scale, and the resultant solids were combined and dried under vacuum at 45° C. to give 2-(((benzyloxy)carbonyl)amino)-3-(1-methyl-1H-pyrazol-4-yl)propanoic acid, enantiomerically enriched with the (R)-enantiomer (5.00 kg, 77% yield) as a black solid. ES-MS m/z 304.1 (M+H).

The material was divided into five equal portions. Each portion (1.00 kg, 3.30 mol) was dissolved in acetone (20 L) and stirred for 10 min at 25° C., before adding (1S,2R)-2-amino-1,2-diphenylethan-1-ol (633 g, 2.97 mol). After stirring at 25° C. for 12 h, the mixtures were filtered, and the isolated solids from the five reactions were combined to give the (1S,2R)-2-amino-1,2-diphenylethan-1-ol salt of the title compound (5.40 kg, 63%) as a white solid. A 0° C. solution of the salt (1.01 kg, 1.96 mol) in water (15 L) was adjusted to pH 9 with 20% aq. Na₂CO₃ and extracted with EtOAc (3×4 L) to remove neutral impurities. The aq. phase was cooled to 0° C., adjusted to pH 2 with H₂SO₄, and extracted with EtOAc (3×4 L). The organic layers were combined, dried over Na₂SO₄, filtered, and concentrated under reduced pressure. The residue was triturated by mixing with EtOAc (10.0 L) at 20° C. for 2 h before filtering to collect the solids. This reaction was repeated five times on the same scale, and the isolated material from all six reactions was combined to give the title compound (2.00 kg, 56% yield) as a white solid. ES-MS m/z 304.1 (M+H). [a]_{D}²⁰=−4.98° (C=1.0, ACN:H₂O, 5:1). >98% ee [Chiral SFC—column: Chiralpak® AD-3, 150× 4.6 mm, 3 μm; column temperature: 35° C.; mobile phase: gradient 10-50% IPA (with 0.1% IPAm) in CO₂; flow rate: 2.5 mL/min].

### Preparation 101

### Benzyl (R)-(1-((1-(4-fluorophenyl)-2-methylpropan-2-yl)amino)-3-(1-methyl-1H-pyrazol-4-yl)-1-oxopropan-2-yl)carbamate

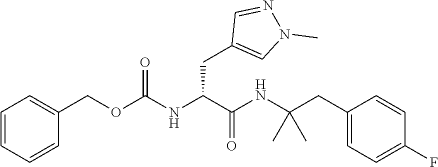 [SMILES: Cn1cc(C[C@@H](NC(=O)OCc2ccccc2)C(=O)NC(C)(C)Cc2ccc(F)cc2)cn1]

To a solution of (R)-2-(((benzyloxy)carbonyl)amino)-3-(1-methyl-1H-pyrazol-4-yl)propanoic acid (6.5 g, 21 mmol), 1-(4-fluorophenyl)-2-methylpropan-2-amine (3.6 g, 21 mmol) and HATU (9.8 g, 26 mmol) in DMA (50 mL) was added DIEA (8.3 g, 11 mL, 64 mmol). The mixture was stirred at 25° C. for 16 h, then was poured into water (50 mL) and extracted with EtOAc (100 mL×3). The combined organic layers were washed with water (50 mL×5), dried over Na₂SO₄ and concentrated. The crude product was purified on silica, eluting with 0-60% EtO Ac in PE to obtain the title compound (9.09 g, 88%) as a brown oil. ES-MS m/z 453.3 (M+H).

### Preparation 102

### (R)-2-Amino-N-(1-(4-fluorophenyl)-2-methylpropan-2-yl)-3-(1-methyl-1H-pyrazol-4-yl)propanamide

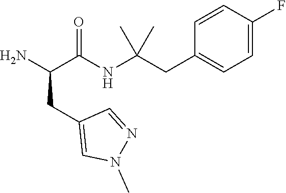 [SMILES: Cn1cc(C[C@@H](N)C(=O)NC(C)(C)Cc2ccc(F)cc2)cn1]

To a slurry of 5% Pd/C (wet) (1 g, 0.5 mmol) in EtOAc (25 mL) was added a solution of benzyl (R)-(1-((1-(4-fluorophenyl)-2-methylpropan-2-yl)amino)-3-(1-methyl-1H-pyrazol-4-yl)-1-oxopropan-2-yl)carbamate (22 g, 49 mmol) in MeOH (200 mL). The reaction was stirred at RT under 45 psi H₂ overnight. An additional portion of 5% Pd/C (1 g, 0.5 mmol) was added and the reaction was stirred for 3 h. The mixture was filtered, and the filtrate was concentrated to afford the title compound (14.7 g, 95%) as a colorless oil. ES-MS m/z 319.2 (M+H).

### Preparation 103

### (R)-2-Amino-N-(1-(4-fluorophenyl)-2-methylpropan-2-yl)-3-(1-methyl-1H-pyrazol-4-yl)propanamide dihydrochloride

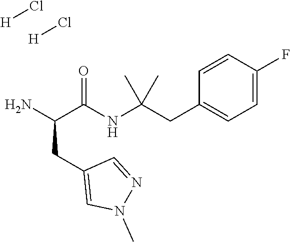

(R)-2-amino-N-(1-(4-fluorophenyl)-2-methylpropan-2-yl)-3-(1-methyl-1H-pyrazol-4-yl)propanamide (14.7 g, 46.2 mmol) was dissolved in MTBE (150 mL). 4N HCl/dioxane (3.5 g, 24 mL, 96 mmol) was added and the reaction was stirred at RT for 30 minutes. The solid was filtered, washed with Et₂O, and dried under vacuum to give the title compound (17.5 g, 44.7 mmol, 97%) as a white solid.

### Preparation 104

### Methyl (R)-2-amino-3-(1-methyl-1H-pyrazol-4-yl)propanoate hydrochloride

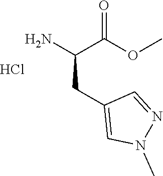

A solution of (R)-2-(((benzyloxy)carbonyl)amino)-3-(1-methyl-1H-pyrazol-4-yl)propanoic acid (180 g, 593 mmol) in MeOH (1.26 L) was treated with SOCl₂ (141 g, 1.19 mol), and the resulting mixture was stirred at 70° C. for 1 h. The reaction mixture was filtered, and the filtrate was concentrated under reduced pressure. The residue was dissolved in MeOH (1.0 L) and Pd/C (50 wt %, 100 g) was added. The suspension was degassed and purged with H₂ (3×) and stirred under H₂ (20 psi) at 25° C. for 12 h. The reaction mixture was filtered, and the filtrate was concentrated under reduced pressure to obtain the title compound (130 g, 87%) as a white solid. ES-MS m/z 184.1 (M+H).

### Preparation 105

### 5-Bromo-8-fluoroisochromane

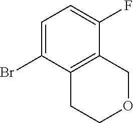 [SMILES: Fc1ccc(Br)c2c1COCC2]

To a solution of 2-(2-bromo-5-fluorophenyl)ethan-1-ol (2.40 kg, 10.9 mol) and 1,3,5-trioxane (1.15 kg, 12.8 mol) in DCM (16.8 L) was added TiCl₄ (4.04 kg, 21.3 mol) dropwise at 0° C. for 1 h, then stirred at 25° C. for 12 h. The reaction was quenched by dropwise water (6.00 L). Then the aqueous phase was extracted with DCM (5.00 L). The mixture was concentrated under reduced pressure. Then the mixture was diluted with EtOAc (10.0 L) and water (3.00 L), the phases were separated and the combined organics were washed with sat. aq. NaCl (3.00 L), dried over Na₂SO₄, filtered and concentrated to obtain the title compound (2400 g) as off-white solid. ¹H-NMR (400 MHz, DMSO-d₆) δ 7.54 (dd, J=8.4, 5.2 Hz, 1H), 7.05 (t, J=9.2 Hz, 1H), 4.69 (s, 1H), 3.90 (t, J=5.6 Hz, 2H), 2.67 (t, J=5.6 Hz, 2H).

### Preparation 106

### 5-Bromo-8-fluoroisochroman-1-one

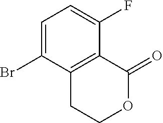 [SMILES: O=C1OCCc2c(Br)ccc(F)c21]

To a solution of 5-bromo-8-fluoroisochromane (2300 g, 2.16 mol) in ACN/water=3/1 (23.0 L) was added tetrabutylammonium iodide (718 g, 2.13 mol) and tert-butyl hydroperoxide (3.43 kg, 26.6 mol, 380 mL, 70% purity) dropwise at 25° C. for 1 h, then stirred at 80° C. for 12 h. The reaction was cooled 25° C. and quenched by sat. Na₂SO₃ (9.20 L) and extracted with DCM (9.20 L). The combined organic phase was dried with anhydrous Na₂SO₄, filtered and concentrated in vacuum. The residue was purified by trituration with MeOH (6000 mL) at 60-25° C. for 120 mins and then triturated again with MTBE (7500 mL) at 25° C. for 60 mins to obtain the title compound (1030 g, 39%) as white solid. 1H-NMR (400 MHz, DMSO-d₆) δ 7.95 (d, J=9.2 Hz, 1H), 7.28 (dd, J=10.4 Hz, 1H), 4.49 (t, J=6.0 Hz, 2H), 3.08 (t, J=6 Hz, 2H).

### Preparation 107

### 3-Bromo-2-(2-chloroethyl)-6-fluorobenzoyl chloride

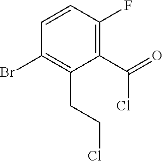 [SMILES: O=C(Cl)c1c(F)ccc(Br)c1CCCl]

To a solution of 5-bromo-8-fluoroisochroman-1-one (10.0 g, 40.8 mmol) in DCE (100 mL), was added benzyl(triethyl)ammonium chloride (18.7 g, 82.1 mmol) and thionyl chloride (15.0 mL, 204 mmol) followed by boron trifluoride etherate (10.2 mL, 81.4 mmol). The mixture was sealed in a glass pressure vessel and heated at 110° C. with stirring for 2 days. The mixture was cooled, opened and diluted with toluene (50 mL) then concentrated under reduced pressure at 45° C. to produce a light-yellow solid. Toluene (50 mL) was added, and the mixture concentrated (3×) to provide the title compound as a light yellow solid with residual benzyl(triethyl)ammonium chloride contributing to the mass. Yield assumed to be 100% (40.8 mmol) and the material was used as-is without further purification.

### Preparation 108

### (R)-3-bromo-2-(2-chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-6-fluorobenzamide

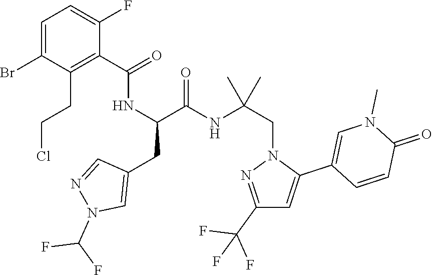 [SMILES: Cn1cc(-c2cc(C(F)(F)F)nn2CC(C)(C)NC(=O)[C@@H](Cc2cnn(C(F)F)c2)NC(=O)c2c(F)ccc(Br)c2CCCl)ccc1=O]

To a solution of (R)-2-amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide hydrochloride (34.0 g, 59.2 mmol) in DCM (296 mL) was added DIEA (41 mL, 0.24 mol). To this solution was added 3-bromo-2-(2-chloroethyl)-6-fluorobenzoyl chloride (60 mmol as the crude mixture prepared in Preparation 107) portionwise over 20 min. The solution was stirred for 30 min at RT. The mixture was concentrated under reduced pressure to yield a thick oil, diluted with EtOAc (700 mL) and sequentially washed with water, 0.1N HCl, sat. NaHCO₃ and sat. aq. NaCl. The organic phase was dried over magnesium sulfate and concentrated under reduced pressure to provide the title compound (46.0 g, 97%) as a light tan foam solid. ES-MS m/z (⁷⁹Br/⁸¹Br, ³⁵Cl/³⁷Cl) 764.0/766.0 (M+H).

The compounds in the following table were prepared in similar manner as described in Preparation 108 using the appropriate primary amine and either TEA or DIEA as base in a suitable solvent like DCM, THF or DMA. Reactants can be added in different orders or in differing equivalency, and purification methods were adjusted to suit the compounds. Such variances would be apparent to one skilled in the art.

TABLE 19

| Preparation | Chemical Name | Structure | ES-MS m/z |
|---|---|---|---|
| 109 | (R)-3-Bromo-2-(2- chloroethyl)-N-(3-(1- (difluoromethyl)-1H- pyrazol-4-yl)-1-((1-(5-(5- fluoro-1-methyl-6-oxo- 1,6-dihydropyridin-3-yl)- 3-(trifluoromethyl)-1H- pyrazol-1-yl)-2- methylpropan-2- yl)amino)-1-oxopropan-2- yl)-6-fluorobenzamide | 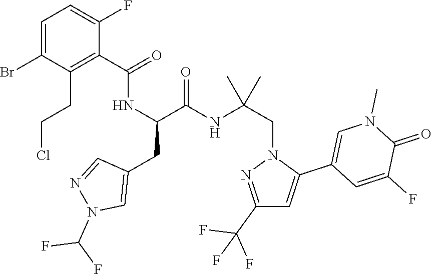 [SMILES: Cn1cc(-c2cc(C(F)(F)F)nn2CC(C)(C)NC(=O)[C@@H](Cc2cnn(C(F)F)c2)NC(=O)c2c(F)ccc(Br)c2CCCl)cc(F)c1=O] | (⁷⁹Br/⁸¹Br, ³⁵Cl/³⁷Cl) 782.4/784.2/ 785.4 (M + H) |
| 110 | (R)-3-Bromo-2-(2- chloroethyl)-6-fluoro-N- (1-((2-methyl-1-(5-(1- methyl-6-oxo-1,6- dihydropyridin-3-yl)-3- (trifluoromethyl)-1H- pyrazol-1-yl)propan-2- yl)amino)-3-(1-methyl- 1H-pyrazol-4-yl)-1- oxopropan-2- yl)benzamide | 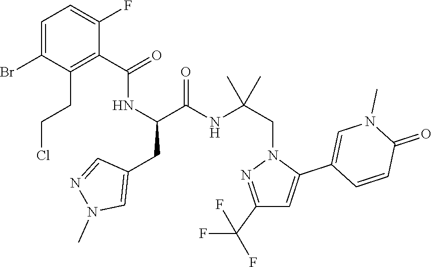 [SMILES: Cn1cc(C[C@@H](NC(=O)c2c(F)ccc(Br)c2CCCl)C(=O)NC(C)(C)Cn2nc(C(F)(F)F)cc2-c2ccc(=O)n(C)c2)cn1] | (¹⁹Br/⁸¹Br, ³⁵Cl/³⁷Cl) 728.2/730.2/ 731.2 (M + H) |
| 111 | 3-Bromo-2-(2- chloroethyl)-6-fluoro-N- ((2R)-1-((2-methyl-1-(5- (2-(tetrahydro-2H-pyran- 2-yl)-2H-1,2,3-triazol-4- yl)-3-(trifluoromethyl)- 1H-pyrazol-1-yl)propan-2- yl)amino)-3-(1-methyl- 1H-pyrazol-4-yl)-1- oxopropan-2- yl)benzamide | 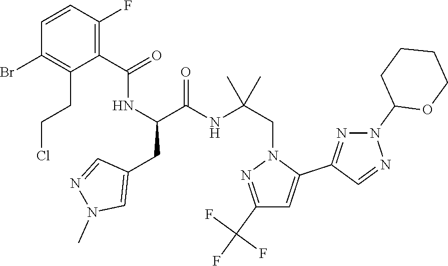 [SMILES: Cn1cc(C[C@@H](NC(=O)c2c(F)ccc(Br)c2CCCl)C(=O)NC(C)(C)Cn2nc(C(F)(F)F)cc2-c2cnn(C3CCCCO3)n2)cn1] | (⁷⁹Br/⁸¹Br, ³⁵Cl/³⁷Cl) 774.0 (M + H) |
| 112 | (R)-N-(1-((1-(5-(2H-1,2,3- Triazol-4-yl)-3- (trifluoromethyl)-1H- pyrazol-1-yl)-2- methylpropan-2- yl)amino)-3-(1-methyl- 1H-pyrazol-4-yl)-1- oxopropan-2-yl)-3-bromo- 2-(2-chloroethyl)-6- fluorobenzamide | 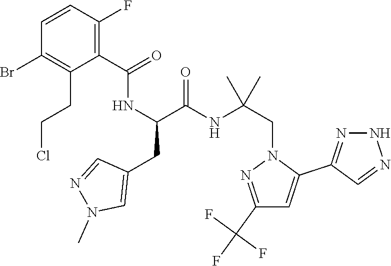 [SMILES: Cn1cc(C[C@@H](NC(=O)c2c(F)ccc(Br)c2CCCl)C(=O)NC(C)(C)Cn2nc(C(F)(F)F)cc2-c2cn[nH]n2)cn1] | (⁷⁹Br/⁸¹Br, ³⁵Cl/³⁷Cl) 690.0 (M + H) |
| 113 | (R)-N-(1-((1-(5-(2H-1,2,3- Triazol-4-yl)-3- (trifluoromethyl)-1H- pyrazol-1-yl)-2- methylpropan-2- yl)amino)-3-(1- (difluoromethyl)-1H- pyrazol-4-yl)-1- oxopropan-2-yl)-3-bromo- 2-(2-chloroethyl)-6- fluorobenzamide | 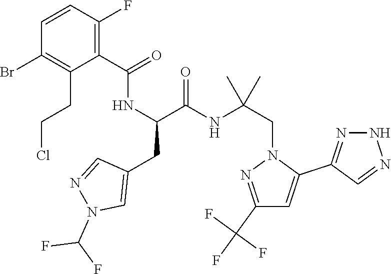 [SMILES: CC(C)(Cn1nc(C(F)(F)F)cc1-c1cn[nH]n1)NC(=O)[C@@H](Cc1cnn(C(F)F)c1)NC(=O)c1c(F)ccc(Br)c1CCCl] | (¹⁹Br/⁸¹Br, ³⁵Cl/³⁷Cl) 725.7 (M + H) |
| 114 | (R)-3-Bromo-2-(2- chloroethyl)-6-fluoro-N- (1-((1-(5-(5-methoxy- 1,2,4-thiadiazol-3-yl)-3- (trifluoromethyl)-1H- pyrazol-1-yl)-2- methylpropan-2- yl)amino)-3-(1-methyl- 1H-pyrazol-4-yl)-1- oxopropan-2- yl)benzamide | 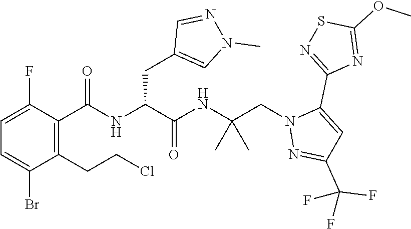 [SMILES: COc1nc(-c2cc(C(F)(F)F)nn2CC(C)(C)NC(=O)[C@@H](Cc2cnn(C)c2)NC(=O)c2c(F)ccc(Br)c2CCCl)ns1] | (¹⁹Br/⁸¹Br, ³⁵Cl/³⁷Cl) 737.3 (M + H) |
| 115 | (R)-3-Bromo-2-(2- chloroethyl)-6-fluoro-N- (1-((1-(5-(5-hydroxy- 1,2,4-thiadiazol-3-yl)-3- (trifluoromethyl)-1H- pyrazol-1-yl)-2- methylpropan-2- yl)amino)-3-(1-methyl- 1H-pyrazol-4-yl)-1- oxopropan-2- yl)benzamide | 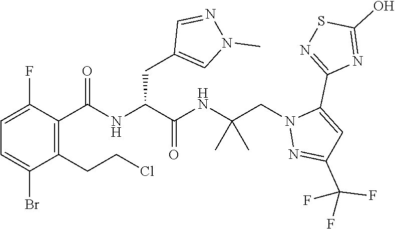 [SMILES: Cn1cc(C[C@@H](NC(=O)c2c(F)ccc(Br)c2CCCl)C(=O)NC(C)(C)Cn2nc(C(F)(F)F)cc2-c2nsc(O)n2)cn1] | (⁷⁹Br/⁸¹Br, ³⁵Cl/³⁷Cl) 723.2 (M + H) |
| 116 | Methyl (R)-2-(3-bromo-2- (2-chloroethyl)-6- fluorobenzamido)-3-(1- methyl-1H-pyrazol-4- yl)propanoate | 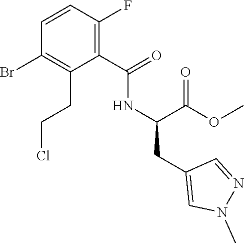 [SMILES: COC(=O)[C@@H](Cc1cnn(C)c1)NC(=O)c1c(F)ccc(Br)c1CCCl] | (⁷⁹Br/⁸¹Br, ³⁵Cl/³⁷Cl) 448.0 (M + H) |
| 117 | Methyl 3-((R)-2-(3- bromo-2-(2-chloroethyl)- 6-fluorobenzamido)-3-(1- methyl-1H-pyrazol-4- yl)propanamido)-3- methyl-2-(3- (trifluoromethyl)phenyl)bu- tanoate | 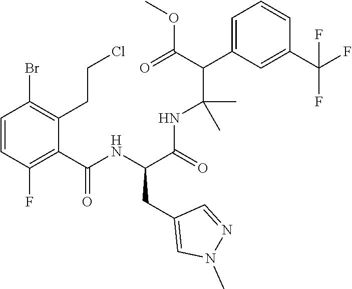 [SMILES: COC(=O)C(c1cccc(C(F)(F)F)c1)C(C)(C)NC(=O)[C@@H](Cc1cnn(C)c1)NC(=O)c1c(F)ccc(Br)c1CCCl] | (¹⁹Br/⁸¹Br, ³⁵Cl/³⁷Cl) 691.0 (M + H) |
| 118 | Methyl 3-((R)-2-(3- bromo-2-(2-chloroethyl)- 6-fluorobenzamido)-3-(1- methyl-1H-pyrazol-4- yl)propanamido)-2-(4- fluorophenyl)-3- methylbutanoate | 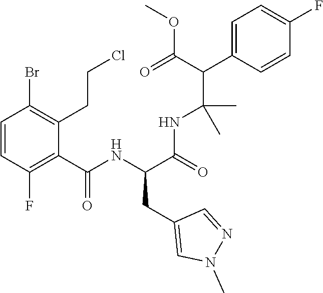 [SMILES: COC(=O)C(c1ccc(F)cc1)C(C)(C)NC(=O)[C@@H](Cc1cnn(C)c1)NC(=O)c1c(F)ccc(Br)c1CCCl] | (⁷⁹Br/⁸¹Br, ³⁵Cl/³⁷Cl) 662.9 (M + Na) |
| 119 | Ethyl 3-((R)-2-(3-bromo- 2-(2-chloroethyl)-6- fluorobenzamido)-3-(1- methyl-1H-pyrazol-4- yl)propanamido)-3- methyl-2-(4- (trifluoromethyl)-1H- pyrazol-1-yl)butanoate | 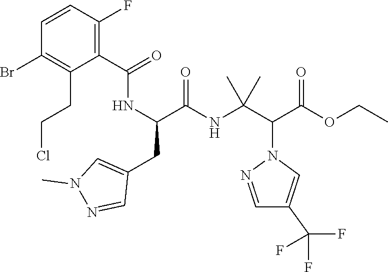 [SMILES: CCOC(=O)C(n1cc(C(F)(F)F)cn1)C(C)(C)NC(=O)[C@@H](Cc1cnn(C)c1)NC(=O)c1c(F)ccc(Br)c1CCCl] | (⁷⁹Br/⁸¹Br, ³⁵Cl/³⁷Cl) 695.0 (M + H) |
| 120 | (R)-3-Bromo-2-(2- chloroethyl)-6-fluoro-N- (1-((1-(5-fluoropyridin-2- yl)-2-methylpropan-2- yl)amino)-3-(1-methyl- 1H-pyrazol-4-yl)-1- oxopropan-2- yl)benzamide | 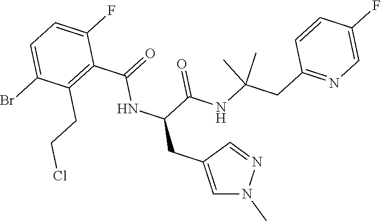 [SMILES: Cn1cc(C[C@@H](NC(=O)c2c(F)ccc(Br)c2CCCl)C(=O)NC(C)(C)Cc2ccc(F)cn2)cn1] | (⁷⁹Br/⁸¹Br, ³⁵Cl/³⁷Cl) 584.0 (M + H) |
| 121 | (R)-3-Bromo-2-(2- chloroethyl)-N-(1-((1-(4- fluorophenyl)-2- methylpropan-2- yl)amino)-3-(1-methyl- 1H-pyrazol-4-yl)-1- oxopropan-2- yl)benzamide | 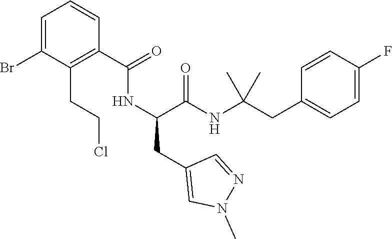 [SMILES: Cn1cc(C[C@@H](NC(=O)c2cccc(Br)c2CCCl)C(=O)NC(C)(C)Cc2ccc(F)cc2)cn1] | (¹⁹Br/⁸¹Br, ³⁵Cl/³⁷Cl) 563.2/565.2/ 567.2 (M + H) |
| 122 | (R)-3-Bromo-2-(2- chloroethyl)-N-(1-((1-(5- cyclopropyl-1H-pyrazol- 1-yl)-2-methylpropan-2- yl)amino)-3-(1-methyl- 1H-pyrazol-4-yl)-1- oxopropan-2-yl)-6- fluorobenzamide | 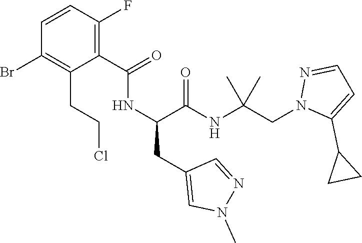 [SMILES: Cn1cc(C[C@@H](NC(=O)c2c(F)ccc(Br)c2CCCl)C(=O)NC(C)(C)Cn2nccc2C2CC2)cn1] | (¹⁹Br/⁸¹Br, ³⁵Cl/³⁷Cl) 595.2 (M + H) |

### Preparation 123

### (R)-2-(5-Bromo-8-fluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

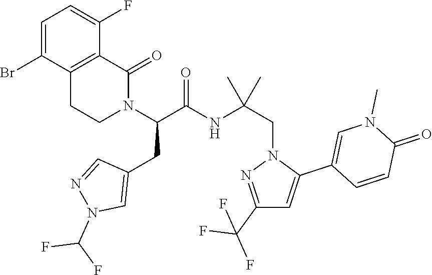 [SMILES: Cn1cc(-c2cc(C(F)(F)F)nn2CC(C)(C)NC(=O)[C@@H](Cc2cnn(C(F)F)c2)N2CCc3c(Br)ccc(F)c3C2=O)ccc1=O]

To a solution of (R)-3-bromo-2-(2-chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-6-fluorobenzamide (46.0 g, 57.1 mmol) in ACN (350 mL) was added cesium carbonate (56.0 g, 172 mmol) and stirred at 40° C. for 18 h then diluted with EtOAc (500 mL) and washed with water (1.5 L). The aqueous phase was then extracted with EtOAc (300 mL) and the two organic phases were combined then sequentially washed with water (500 mL) and sat. aq. NaCl (500 mL). To the organics was added 1 N HCl (100 mL) and the mixture was shaken in a separatory funnel for 2 min and the organics separated and washed with sat NaHCO₃. dried over MgSO₄, filtered and concentrated under reduced pressure. The material was purified on silica gel, eluting with 50-100% EtOAc in cyclohexane followed by 5% MeOH in EtOAc to obtain the title compound (33.0 g, 79%) as a yield a white foam solid. ES-MS m/z (⁷⁹Br/⁸¹Br) 728.2/730.2 (M+H).

The compounds in the following table were prepared in similar manner as described in Preparation 123 using the appropriate secondary amide and either potassium carbonate or cesium carbonate in a suitable solvent such as NMP, ACN, DMF, DMA or THF. The reactions were run at temperatures ranging from ambient to 70° C. and for a duration of 1 to 18 h. An aqueous 1 N HCl wash of the organics can reduce certain impurities but is not required for the preparation. Reactants can be added in different orders or in differing equivalency, and purification methods were adjusted to suit the compounds. Such variances would be apparent to one skilled in the art.

TABLE 20

| Prepara- | | | |
|---|---|---|---|
| tion | Chemical Name | Structure | ES-MS m/z |
| 124 | (R)-2-(5-Bromo-8-fluoro-1- oxo-3,4-dihydroisoquinolin- 2(1H)-yl)-3-(1- (difluoromethyl)-1H- pyrazol-4-yl)-N-(1-(5-(5- fluoro-1-methyl-6-oxo-1,6- dihydropyridin-3-yl)-3- (trifluoromethyl)-1H- pyrazol-1-yl)-2- methylpropan-2- yl)propanamide | 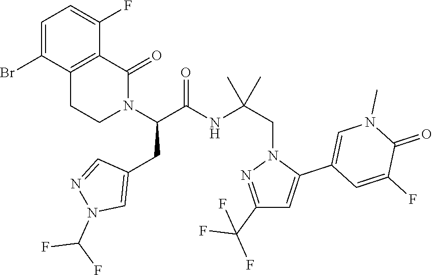 [SMILES: Cn1cc(-c2cc(C(F)(F)F)nn2CC(C)(C)NC(=O)[C@@H](Cc2cnn(C(F)F)c2)N2CCc3c(Br)ccc(F)c3C2=O)cc(F)c1=O] | (⁷⁹Br/⁸¹Br) 746.2/748.2 (M + H) |
| 125 | (R)-2-(5-Bromo-8-fluoro-1- oxo-3,4-dihydroisoquinolin- 2(1H)-yl)-N-(2-methyl-1-(5- (1-methyl-6-oxo-1,6- dihydropyridin-3-yl)-3- (trifluoromethyl)-1H- pyrazol-1-yl)propan-2-yl)-3- (1-methyl-1H-pyrazol-4- yl)propanamide | 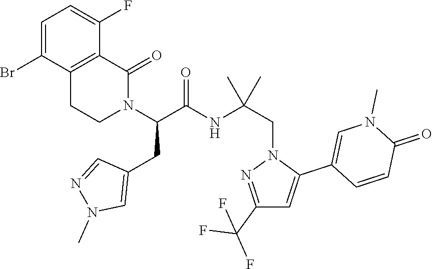 [SMILES: Cn1cc(C[C@H](C(=O)NC(C)(C)Cn2nc(C(F)(F)F)cc2-c2ccc(=O)n(C)c2)N2CCc3c(Br)ccc(F)c3C2=O)cn1] | (⁷⁹Br/⁸¹Br) 692.2/694.2 (M + H) |
| 126 | (2R)-2-(5-Bromo-8-fluoro- 1-oxo-3,4- dihydroisoquinolin-2(1H)- yl)-N-(2-methyl-1-(5-(2- (tetrahydro-2H-pyran-2-yl)- 2H-1,2,3-triazol-4-yl)-3- (trifluoromethyl)-1H- pyrazol-1-yl)propan-2-yl)-3- (1-methyl-1H-pyrazol-4- yl)propanamide | 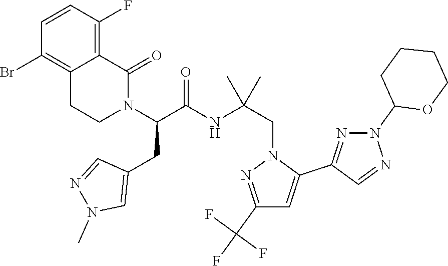 [SMILES: Cn1cc(C[C@H](C(=O)NC(C)(C)Cn2nc(C(F)(F)F)cc2-c2cnn(C3CCCCO3)n2)N2CCc3c(Br)ccc(F)c3C2=O)cn1] | (⁷⁹Br/⁸¹Br) 736.2 (M + H) |
| 127 | (R)-N-(1-(5-(2H-1,2,3- Triazol-4-yl)-3- (trifluoromethyl)-1H- pyrazol-1-yl)-2- methylpropan-2-yl)-2-(5- bromo-8-fluoro-1-oxo-3,4- dihydroisoquinolin-2(1H)- yl)-3-(1-methyl-1H-pyrazol- 4-yl)propanamide | 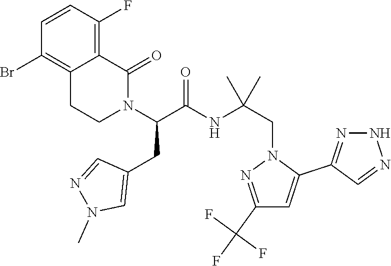 [SMILES: Cn1cc(C[C@H](C(=O)NC(C)(C)Cn2nc(C(F)(F)F)cc2-c2cn[nH]n2)N2CCc3c(Br)ccc(F)c3C2=O)cn1] | (⁷⁹Br/⁸¹Br) 676.0 (M + Na) |
| 128 | (R)-N-(1-(5-(2H-1,2,3- Triazol-4-yl)-3- (trifluoromethyl)-1H- pyrazol-1-yl)-2- methylpropan-2-yl)-2-(5- bromo-8-fluoro-1-oxo-3,4- dihydroisoquinolin-2(1H)- yl)-3-(1-(difluoromethyl)- 1H-pyrazol-4- yl)propanamide | 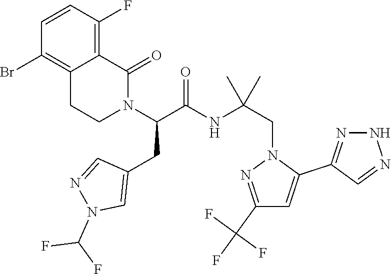 [SMILES: CC(C)(Cn1nc(C(F)(F)F)cc1-c1cn[nH]n1)NC(=O)[C@@H](Cc1cnn(C(F)F)c1)N1CCc2c(Br)ccc(F)c2C1=O] | (⁷⁹Br/⁸¹Br) 690.0 (M + H) |
| 129 | (R)-2-(5-Bromo-8-fluoro-1- oxo-3,4-dihydroisoquinolin- 2(1H)-yl)-N-(1-(5-(5- methoxy-1,2,4-thiadiazol-3- yl)-3-(trifluoromethyl)-1H- pyrazol-1-yl)-2- methylpropan-2-yl)-3-(1- methyl-1H-pyrazol-4- yl)propanamide | 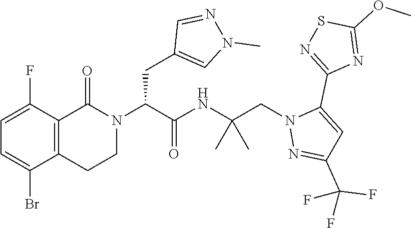 [SMILES: COc1nc(-c2cc(C(F)(F)F)nn2CC(C)(C)NC(=O)[C@@H](Cc2cnn(C)c2)N2CCc3c(Br)ccc(F)c3C2=O)ns1] | (⁷⁹Br/⁸¹Br) 723.2 (M + Na) |
| 130 | (R)-2-(5-Bromo-8-fluoro-1- oxo-3,4-dihydroisoquinolin- 2(1H)-yl)-N-(1-(5-(5- hydroxy-1,2,4-thiadiazol-3- yl)-3-(trifluoromethyl)-1H- pyrazol-1-yl)-2- methylpropan-2-yl)-3-(1- methyl-1H-pyrazol-4- yl)propanamide | 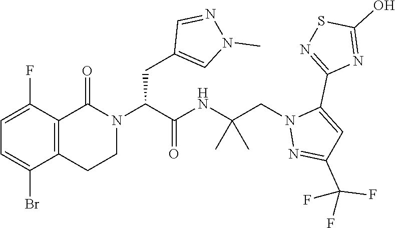 [SMILES: Cn1cc(C[C@H](C(=O)NC(C)(C)Cn2nc(C(F)(F)F)cc2-c2nsc(O)n2)N2CCc3c(Br)ccc(F)c3C2=O)cn1] | (⁷⁹Br/⁸¹Br) 687.1 (M + H) |
| 131 | Methyl (R)-2-(5-bromo-8- fluoro-1-oxo-3,4- dihydroisoquinolin-2(1H)- yl)-3-(1-methyl-1H-pyrazol- 4-yl)propanoate | 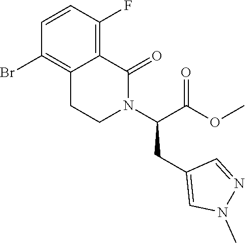 [SMILES: COC(=O)[C@@H](Cc1cnn(C)c1)N1CCc2c(Br)ccc(F)c2C1=O] | (⁷⁹Br/⁸¹Br) 409.9 (M + H) |
| 132 | Methyl 3-((R)-2-(5-bromo- 8-fluoro-1-oxo-3,4- dihydroisoquinolin-2(1H)- yl)-3-(1-methyl-1H-pyrazol- 4-yl)propanamido)-3- methyl-2-(3- (trifluoromethyl)phenyl)buta- noate | 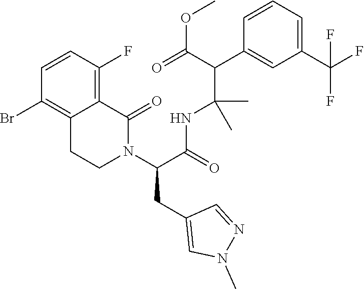 [SMILES: COC(=O)C(c1cccc(C(F)(F)F)c1)C(C)(C)NC(=O)[C@@H](Cc1cnn(C)c1)N1CCc2c(Br)ccc(F)c2C1=O] | (⁷⁹Br/⁸¹Br) 655.0 (M + H) |
| 133 | Methyl 3-((R)-2-(5-bromo- 8-fluoro-1-oxo-3,4- dihydroisoquinolin-2(1H)- yl)-3-(1-methyl-1H-pyrazol- 4-yl)propanamido)-2-(4- fluorophenyl)-3- methylbutanoate | 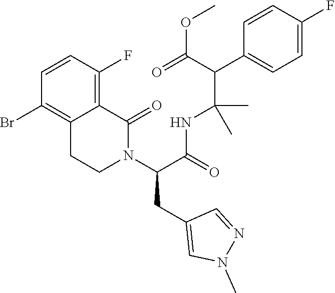 [SMILES: COC(=O)C(c1ccc(F)cc1)C(C)(C)NC(=O)[C@@H](Cc1cnn(C)c1)N1CCc2c(Br)ccc(F)c2C1=O] | (⁷⁹Br/⁸¹Br) 604.9 (M + H) |
| 134 | Ethyl 3-((R)-2-(5-bromo-8- fluoro-1-oxo-3,4- dihydroisoquinolin-2(1H)- yl)-3-(1-methyl-1H-pyrazol- 4-yl)propanamido)-3- methyl-2-(4- (trifluoromethyl)-1H- pyrazol-1-yl)butanoate | 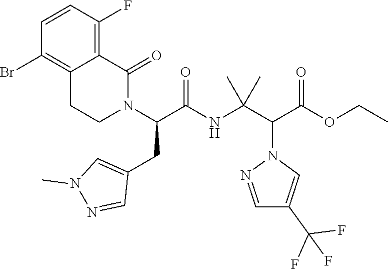 [SMILES: CCOC(=O)C(n1cc(C(F)(F)F)cn1)C(C)(C)NC(=O)[C@@H](Cc1cnn(C)c1)N1CCc2c(Br)ccc(F)c2C1=O] | (⁷⁹Br/⁸¹Br) 659.0 (M + H) |
| 135 | (R)-2-(5-Bromo-8-fluoro-1- oxo-3,4-dihydroisoquinolin- 2(1H)-yl)-N-(1-(5- fluoropyridin-2-yl)-2- methylpropan-2-yl)-3-(1- methyl-1H-pyrazol-4- yl)propanamide | 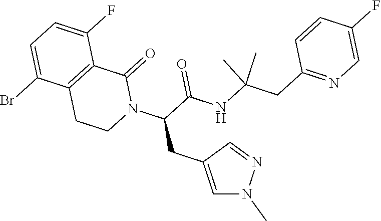 [SMILES: Cn1cc(C[C@H](C(=O)NC(C)(C)Cc2ccc(F)cn2)N2CCc3c(Br)ccc(F)c3C2=O)cn1] | (⁷⁹Br/⁸¹Br) 545.9 (M + H) |
| 136 | (R)-2-(5-Bromo-1-oxo-3,4- dihydroisoquinolin-2(1H)- yl)-N-(1-(4-fluorophenyl)-2- methylpropan-2-yl)-3-(1- methyl-1H-pyrazol-4- yl)propanamide | 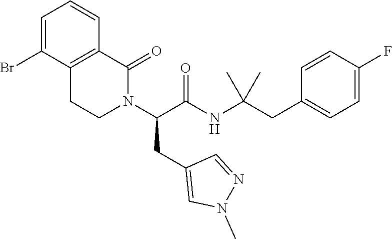 [SMILES: Cn1cc(C[C@H](C(=O)NC(C)(C)Cc2ccc(F)cc2)N2CCc3c(Br)cccc3C2=O)cn1] | (⁷⁹Br/⁸¹Br) 527.2/529.2 (M + H) |
| 137 | (R)-2-(5-Bromo-8-fluoro-1- oxo-3,4-dihydroisoquinolin- 2(1H)-yl)-N-(1-(5- cyclopropyl-1H-pyrazol-1- yl)-2-methylpropan-2-yl)-3- (1-methyl-1H-pyrazol-4- yl)propanamide | 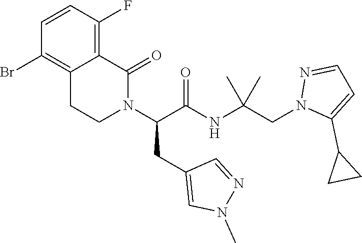 [SMILES: Cn1cc(C[C@H](C(=O)NC(C)(C)Cn2nccc2C2CC2)N2CCc3c(Br)ccc(F)c3C2=O)cn1] | (⁷⁹Br/⁸¹Br) (M + H) |

### Preparation 138

### (R)-3-(1-(Difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

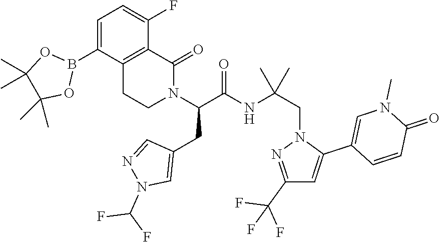 [SMILES: Cn1cc(-c2cc(C(F)(F)F)nn2CC(C)(C)NC(=O)[C@@H](Cc2cnn(C(F)F)c2)N2CCc3c(B4OC(C)(C)C(C)(C)O4)ccc(F)c3C2=O)ccc1=O]

To a mixture of (R)-2-(5-bromo-8-fluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (1.5 g, 2.1 mmol), bis(pinacolato)diboron (1.0 g, 4.1 mmol), KOAc (0.61 g, 6.2 mmol) and Pd (dppf) Cl₂·CH₂Cl₂ (0.17 g, 0.21 mmol) under N₂ gas atmosphere was added 1,4-dioxane (10 mL). A long needle was inserted below the mixture surface and N₂ gas was bubbled through the mixture for 5 min. The mixture was then heated to 100° C. for 1 h, diluted with DCM, dried over magnesium sulfate and concentrated under reduced pressure. The residue was purified by silica gel, 80-100% EtOAc in cyclohexane. The residue was triturated from EtOAc and cyclohexane to provide an oily solid that was diluted with DCM (20 mL) and concentrated under reduced pressure to provide a tan foam solid. ES-MS m/z 776.6 (M+H).

The compounds in the following table were prepared in similar manner as described in Preparation 138 using the appropriate aryl halide. Reactants can be added in different orders or in differing equivalency, and purification methods were adjusted to suit the compounds. Such variances would be apparent to one skilled in the art.

TABLE 21

| Prepara- | | | ES-MS |
|---|---|---|---|
| tion | Chemical Name | Structure | m/z |
| 139 | (R)-3-(1-(Difluoromethyl)-1H- pyrazol-4-yl)-N-(1-(5-(5- fluoro-1-methyl-6-oxo-1,6- dihydropyridin-3-yl)-3- (trifluoromethyl)-1H-pyrazol- 1-yl)-2-methylpropan-2-yl)-2- (8-fluoro-1-oxo-5-(4,4,5,5- tetramethyl-1,3,2- dioxaborolan-2-yl)-3,4- dihydroisoquinolin-2(1H)- yl)propanamide | 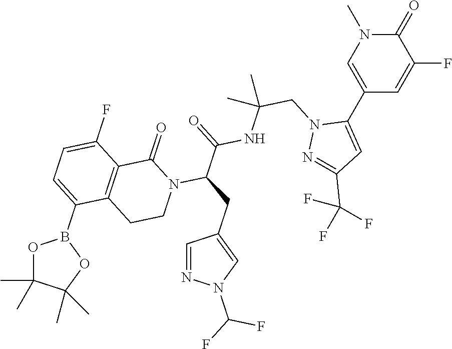 [SMILES: Cn1cc(-c2cc(C(F)(F)F)nn2CC(C)(C)NC(=O)[C@@H](Cc2cnn(C(F)F)c2)N2CCc3c(B4OC(C)(C)C(C)(C)O4)ccc(F)c3C2=O)cc(F)c1=O] | 794.2 (M + H) |
| 140 | (R)-2-(8-Fluoro-1-oxo-5- (4,4,5,5-tetramethyl-1,3,2- dioxaborolan-2-yl)-3,4- dihydroisoquinolin-2(1H)-yl)- N-(2-methyl-1-(5-(1-methyl-6- oxo-1,6-dihydropyridin-3-yl)- 3-(trifluoromethyl)-1H- pyrazol-1-yl)propan-2-yl)-3-(1- methyl-1H-pyrazol-4- yl)propanamide | 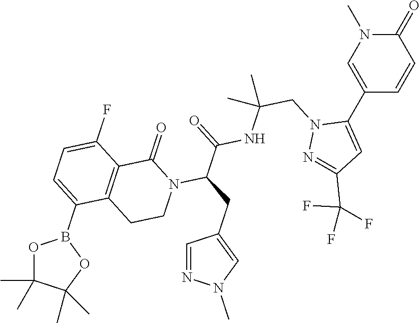 [SMILES: Cn1cc(C[C@H](C(=O)NC(C)(C)Cn2nc(C(F)(F)F)cc2-c2ccc(=O)n(C)c2)N2CCc3c(B4OC(C)(C)C(C)(C)O4)ccc(F)c3C2=O)cn1] | 740.6 (M + H) |
| 141 | Methyl 3-((R)-2-(8-fluoro-1- oxo-5-(4,4,5,5-tetramethyl- 1,3,2-dioxaborolan-2-yl)-3,4- dihydroisoquinolin-2(1H)-yl)- 3-(1-methyl-1H-pyrazol-4- yl)propanamido)-3-methyl-2- (3- (trifluoromethyl)phenyl)butano- ate | 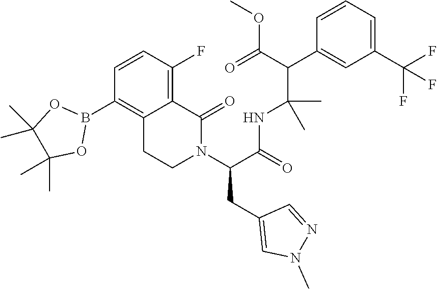 [SMILES: COC(=O)C(c1cccc(C(F)(F)F)c1)C(C)(C)NC(=O)[C@@H](Cc1cnn(C)c1)N1CCc2c(B3OC(C)(C)C(C)(C)O3)ccc(F)c2C1=O] | 701.2 (M + Na) |
| 142 | Ethyl 3-((R)-2-(8-fluoro-1- oxo-5-(4,4,5,5-tetramethyl- 1,3,2-dioxaborolan-2-yl)-3,4- dihydroisoquinolin-2(1H)-yl)- 3-(1-methyl-1H-pyrazol-4- yl)propanamido)-3-methyl-2- (4-(trifluoromethyl)-1H- pyrazol-1-yl)butanoate | 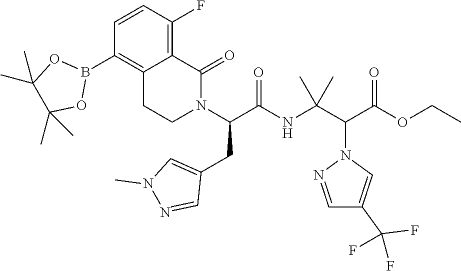 [SMILES: CCOC(=O)C(n1cc(C(F)(F)F)cn1)C(C)(C)NC(=O)[C@@H](Cc1cnn(C)c1)N1CCc2c(B3OC(C)(C)C(C)(C)O3)ccc(F)c2C1=O] | 705.1 (M + H) |
| 143 | (R)-N-(1-(4-Fluorophenyl)-2- methylpropan-2-yl)-3-(1- methyl-1H-pyrazol-4-yl)-1-(1- oxo-5-(4,4,5,5-tetramethyl- 1,3,2-dioxaborolan-2-yl)-3,4- dihydroisoquinolin-2(1H)- yl)propanamide | 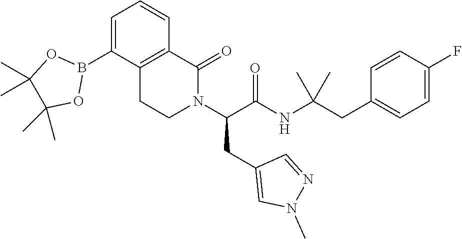 [SMILES: Cn1cc(C[C@H](C(=O)NC(C)(C)Cc2ccc(F)cc2)N2CCc3c(B4OC(C)(C)C(C)(C)O4)cccc3C2=O)cn1] | 575.5 (M + H) |

### Preparation 144

### (R)-(2-(3-(1-(Difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-8-fluoro-1-oxo-1,2,3,4-tetrahydroisoquinolin-5-yl)boronic acid

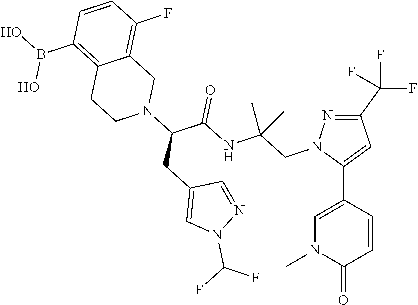 [SMILES: Cn1cc(-c2cc(C(F)(F)F)nn2CC(C)(C)NC(=O)[C@@H](Cc2cnn(C(F)F)c2)N2CCc3c(B(O)O)ccc(F)c3C2)ccc1=O]

To a mixture of (R)-2-(5-bromo-8-fluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (1.4 g, 1.6 mmol) in MeOH (24 mL) was added hypodiboric acid (1.4 g, 16 mmol), a solution of XPhos Pd G3 (0.26 g, 0.31 mmol) in DCE (2.4 mL) and KOAc (0.61 g, 6.2 mmol). The reaction was stirred at 40° C. for 16 h under N₂ atmosphere. The reaction was cooled to RT and filtered. The filtrate was adjusted to ˜ pH 7 with FA and concentrated under reduced pressure. The residue was purified on silica gel, eluting with 0-5% MeOH (with 0.5% FA) in DCM, to give the title compound as a yellow solid (830 mg, 72%). ES-MS m/z 694.2 (M+H).

The compounds in the following table were prepared in similar manner as described in Preparation 144 using the appropriate aryl halide. Reactants can be added in different orders or in differing equivalency, and purification methods were adjusted to suit the compounds. Such variances would be apparent to one skilled in the art.

TABLE 22

| Prepara- | | | ES-MS |
|---|---|---|---|
| tion | Chemical Name | Structure | m/z |
| 145 | (R)-(8-Fluoro-2-(1-((2-methyl- 1-(5-(1-methyl-6-oxo-1,6- dihydropyridin-3-yl)-3- (trifluoromethyl)-1H-pyrazol- 1-yl)propan-2-yl)amino)-3-(1- methyl-1H-pyrazol-4-yl)-1- oxopropan-2-yl)-1-oxo-1,2,3,4- tetrahydroisoquinolin-5- yl)boronic acid | 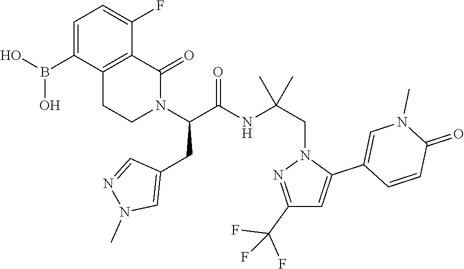 [SMILES: Cn1cc(C[C@H](C(=O)NC(C)(C)Cn2nc(C(F)(F)F)cc2-c2ccc(=O)n(C)c2)N2CCc3c(B(O)O)ccc(F)c3C2=O)cn1] | 657.8 (M + H) |
| 146 | (8-Fluoro-2-((2R)-1-((2- methyl-1-(5-(2-(tetrahydro-2H- pyran-2-yl)-2H-1,2,3-triazol-4- yl)-3-(trifluoromethyl)-1H- pyrazol-1-yl)propan-2- yl)amino)-3-(1-methyl-1H- pyrazol-4-yl)-1-oxopropan-2- yl)-1-oxo-1,2,3,4- tetrahydroisoquinolin-5- yl)boronic acid | 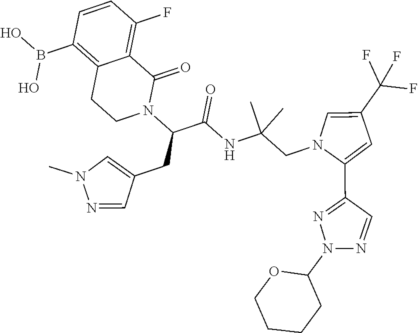 [SMILES: Cn1cc(C[C@H](C(=O)NC(C)(C)Cn2cc(C(F)(F)F)cc2-c2cnn(C3CCCCO3)n2)N2CCc3c(B(O)O)ccc(F)c3C2=O)cn1] | 702.3 (M + H) |
| 147 | (R)-(2-(1-((1-(5-(2H-1,2,3- Triazol-4-yl)-3- (trifluoromethyl)-1H-pyrazol- 1-yl)-2-methylpropan-2- yl)amino)-3-(1-methyl-1H- pyrazol-4-yl)-1-oxopropan-2- yl)-8-fluoro-1-oxo-1,2,3,4- tetrahydroisoquinolin-5- yl)boronic acid | 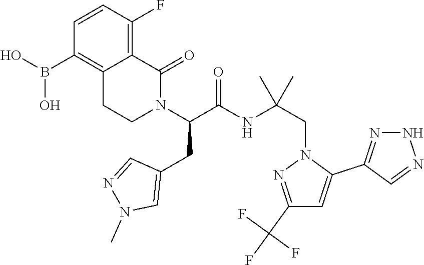 [SMILES: Cn1cc(C[C@H](C(=O)NC(C)(C)Cn2nc(C(F)(F)F)cc2-c2cn[nH]n2)N2CCc3c(B(O)O)ccc(F)c3C2=O)cn1] | 618.3 (M + H) |
| 148 | (R)-(2-(1-((1-(5-(2H-1,2,3- Triazol-4-yl)-3- (trifluoromethyl)-1H-pyrazol- 1-yl)-2-methylpropan-2- yl)amino)-3-(1- (difluoromethyl)-1H-pyrazol- 4-yl)-1-oxopropan-2-yl)-8- fluoro-1-oxo-1,2,3,4- tetrahydroisoquinolin-5- yl)boronic acid | 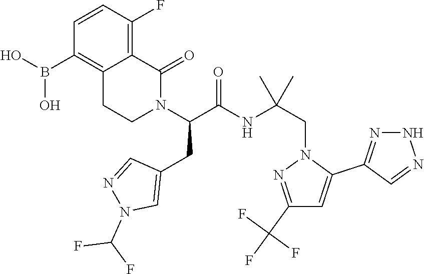 [SMILES: CC(C)(Cn1nc(C(F)(F)F)cc1-c1cn[nH]n1)NC(=O)[C@@H](Cc1cnn(C(F)F)c1)N1CCc2c(B(O)O)ccc(F)c2C1=O] | 654.2 (M + H) |
| 149 | (R)-(8-Fluoro-2-(1-((1-(5-(5- methoxy-1,2,4-thiadiazol-3- yl)-3-(trifluoromethyl)-1H- pyrazol-1-yl)-2-methylpropan- 2-yl)amino)-3-(1-methyl-1H- pyrazol-4-yl)-1-oxopropan-2- yl)-1-oxo-1,2,3,4- tetrahydroisoquinolin-5- yl)boronic acid | 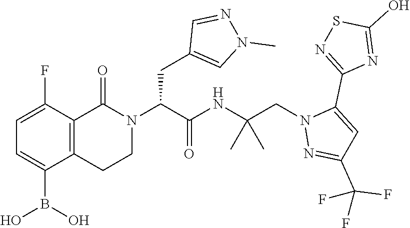 | 665.4 (M + H) |
| 150 | (R)-(8-Fluoro-2-(1-((1-(5-(5- hydroxy-1,2,4-thiadiazol-3-yl)- 3-(trifluoromethyl)-1H- pyrazol-1-yl)-2-methylpropan- 2-yl)amino)-3-(1-methyl-1H- pyrazol-4-yl)-1-oxopropan-2- yl)-1-oxo-1,2,3,4- tetrahydroisoquinolin-5- yl)boronic acid | 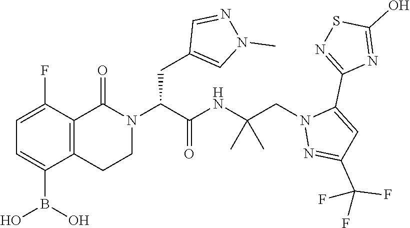 [SMILES: Cn1cc(C[C@H](C(=O)NC(C)(C)Cn2nc(C(F)(F)F)cc2-c2nsc(O)n2)N2CCc3c(B(O)O)ccc(F)c3C2=O)cn1] | 651.4 (M + H) |
| 151ª | (R)-(8-Fluoro-2-(1-methoxy-3- (1-methyl-1H-pyrazol-4-yl)-1- oxopropan-2-yl)-1-oxo-1,2,3,4- tetrahydroisoquinolin-5- yl)boronic acid | 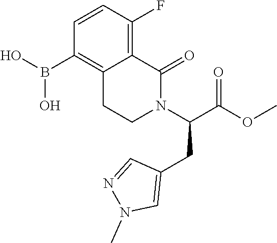 [SMILES: COC(=O)[C@@H](Cc1cnn(C)c1)N1CCc2c(B(O)O)ccc(F)c2C1=O] | 375.9 (M + H) |
| 152ª | (8-Fluoro-2-((2R)-1-((3-(4- fluorophenyl)-4-methoxy-2- methyl-4-oxobutan-2- yl)amino)-3-(1-methyl-1H- pyrazol-4-yl)-1-oxopropan-2- yl)-1-oxo-1,2,3,4- tetrahydroisoquinolin-5- yl)boronic acid | 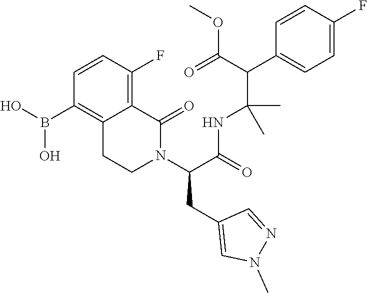 [SMILES: COC(=O)C(c1ccc(F)cc1)C(C)(C)NC(=O)[C@@H](Cc1cnn(C)c1)N1CCc2c(B(O)O)ccc(F)c2C1=O] | 569.3 (M + H) |
| 153ª | (R)-(8-Fluoro-2-(1-((1-(5- fluoropyridin-2-yl)-2- methylpropan-2-yl)amino)-3- (1-methyl-1H-pyrazol-4-yl)-1- oxopropan-2-yl)-1-oxo-1,2,3,4- tetrahydroisoquinolin-5- yl)boronic acid | 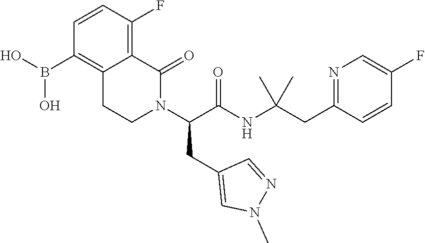 [SMILES: Cn1cc(C[C@H](C(=O)NC(C)(C)Cc2ccc(F)cn2)N2CCc3c(B(O)O)ccc(F)c3C2=O)cn1] | 512.1 (M + H) |
| 154^{a} | (R)-(2-(1-((1-(5-Cyclopropyl- 1H-pyrazol-1-yl)-2- methylpropan-2-yl)amino)-3- (1-methyl-1H-pyrazol-4-yl)-1- oxopropan-2-yl)-8-fluoro-1- oxo-1,2,3,4- tetrahydroisoquinolin-5- yl)boronic acid | 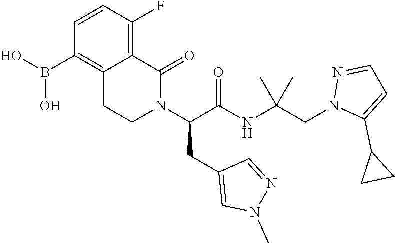 [SMILES: Cn1cc(C[C@H](C(=O)NC(C)(C)Cn2nccc2C2CC2)N2CCc3c(B(O)O)ccc(F)c3C2=O)cn1] | 523.3 (M + H) |

^{a}Chloro[(di(1-adamantyl)-N-butylphosphine)-2-(2-aminobiphenyl)]palladium(II) (0.1 equiv) was used as catalyst along with ethylene glycol (4 equiv); KOAc (3.5 equiv) was used as base; MeOH was used as solvent; and the reaction was heated to 40° C. for 16 h.

### Preparation 155

### (R)-(2-(3-(1-(Difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)-8-fluoro-1-oxo-1,2,3,4-tetrahydroisoquinolin-5-yl)boronic acid

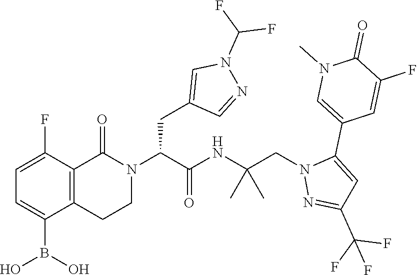 [SMILES: Cn1cc(-c2cc(C(F)(F)F)nn2CC(C)(C)NC(=O)[C@@H](Cc2cnn(C(F)F)c2)N2CCc3c(B(O)O)ccc(F)c3C2=O)cc(F)c1=O]

A 20 ml vial containing a stir bar was charged sequentially with (R)-2-(5-bromo-8-fluoro-1-oxo-3,4-dihydroisoquinolin-2(1H)-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (280 mg, 375 μmol), bis(pinacolato)diborane (191 mg, 752 μmol), KOAc (110 mg 1.12 mmol), and Pd(dppf)Cl₂·CH₂Cl₂ (31 mg, 0.10 Eq, 38 μmol). The reaction mixture was evacuated and backfilled three times with N₂, then 1,4-dioxane (5 mL) was added. The reaction mixture was stirred at 100° C. for 1 h, cooled to RT, filtered, and concentrated under reduced pressure. The residue was purified by reverse-phase flash chromatography (100 g C18 column, solvent A=10 mM ammonium bicarbonate with 5% MeOH in H₂O, solvent B=ACN, gradient 50-70% B) to provide the title compound (75.1 mg, 81% purity, 23% yield) as a light brown solid. ES-MS m/z 712.4 (M+H).

### Preparation 156

### (2R)-3-(1-(Difluoromethyl)-1H-pyrazol-4-yl)-2-(8-fluoro-5-(6-fluoro-1-(tetrahydro-2H-pyran-2-yl)-1H-pyrazolo[4,3-b]pyridin-5-yl)-1-oxo-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

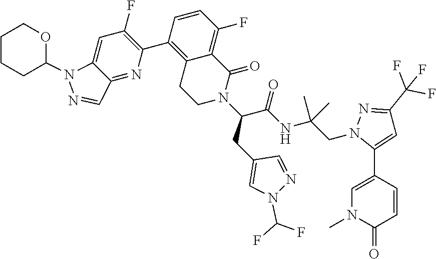 [SMILES: Cn1cc(-c2cc(C(F)(F)F)nn2CC(C)(C)NC(=O)[C@@H](Cc2cnn(C(F)F)c2)N2CCc3c(-c4nc5cnn(C6CCCCO6)c5cc4F)ccc(F)c3C2=O)ccc1=O]

To a solution of (R)-(2-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-8-fluoro-1-oxo-1,2,3,4-tetrahydroisoquinolin-5-yl)boronic acid (135 mg, 183 μmol) in 1,4-dioxane (5 mL), EtOH (5 mL) and water (0.5 mL) was added 5-bromo-6-fluoro-1-(tetrahydro-2H-pyran-2-yl)-1H-pyrazolo[4,3-b]pyridine (68.7 mg, 220 μmol), sodium carbonate (58.2 mg, 39.5 μL, 549 μmol) and XPhos Pd G3 (31.0 mg, 36.6 μmol) at 20° C. The mixture was purged with N₂ 3 times, then stirred at 90° C. for 16 h under N₂. The reaction mixture was then filtered and concentrated. The residue was purified on silica gel, eluting with 0-5% MeOH in DCM to provide the title compound (138 mg, 0.14 mmol, 77% yield, 89% purity) as a brown solid. ES-MS m/z 869.9 (M+H).

The compounds in the following table were prepared in similar manner as described in Preparation 156 using the appropriate aryl halide and boronic acid. Reactants can be added in different orders or in differing equivalency, and purification methods were adjusted to suit the compounds. Such variances would be apparent to one skilled in the art.

TABLE 23

| Prepa- | | | |
|---|---|---|---|
| ration/ | | | |
| Ex- | | | ES-MS |
| ample | Chemical Name | Structure | m/z |
| Prep 157 | (2R)-3-(1- (Difluoromethyl)-1H- pyrazol-4-yl)-2-(8- fluoro-5-(4-fluoro-1- (tetrahydro-2H-pyran-2- yl)-1H-pyrazolo[3,4- c]pyridin-5-yl)-1-oxo- 3,4-dihydroisoquinolin- 2(1H)-yl)-N-(2-methyl- 1-(5-(1-methyl-6-oxo- 1,6-dihydropyridin-3- yl)-3-(trifluoromethyl)- 1H-pyrazol-1-yl)propan- 2-yl)propanamide | 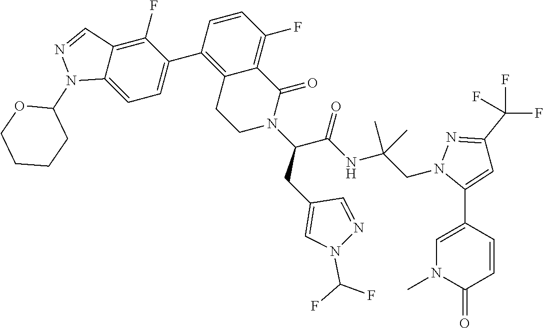 [SMILES: Cn1cc(-c2cc(C(F)(F)F)nn2CC(C)(C)NC(=O)[C@@H](Cc2cnn(C(F)F)c2)N2CCc3c(-c4ccc5c(cnn5C5CCCCO5)c4F)ccc(F)c3C2=O)ccc1=O] | 869.0 (M + H) |
| Ex 161 | (2R)-2-(5-(5-Cyano-1- methyl-1H-indazol-6- yl)-8-fluoro-1-oxo-3,4- dihydroisoquinolin- 2(1H)-yl)-N-(2-methyl- 1-(5-(2-(tetrahydro-2H- pyran-2-yl)-2H-1,2,3- triazol-4-yl)-3- (trifluoromethyl)-1H- pyrazol-1-yl)propan-2- yl)-3-(1-methyl-1H- pyrazol-4- yl)propanamide | 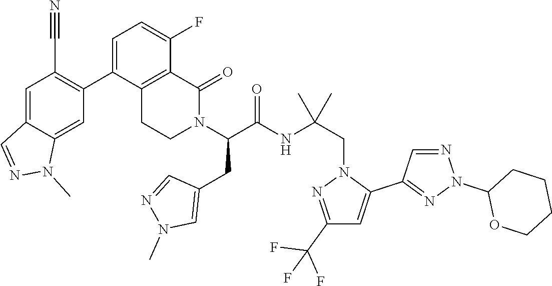 [SMILES: Cn1cc(C[C@H](C(=O)NC(C)(C)Cn2nc(C(F)(F)F)cc2-c2cnn(C3CCCCO3)n2)N2CCc3c(-c4cc5c(cnn5C)cc4C#N)ccc(F)c3C2=O)cn1] | 835.6 (M + Na) |
| Prep 159 | (2R)-2-(5-(6-Cyano-1- (tetrahydro-2H-pyran-2- yl)-1H-pyrazolo[4,3- b]pyridin-5-yl)-8-fluoro- 1-oxo-3,4- dihydroisoquinolin- 2(1H)-yl)-N-(2-methyl- 1-(5-(2-(tetrahydro-2H- pyran-2-yl)-2H-1,2,3- triazol-4-yl)-3- (trifluoromethyl)-1H- pyrazol-1-yl)propan-2- yl)-3-(1-methyl-1H- pyrazol-4- yl)propanamide | 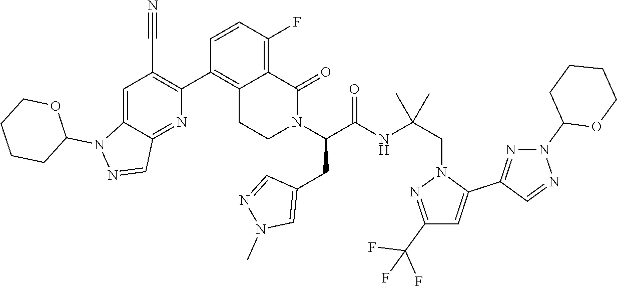 [SMILES: Cn1cc(C[C@H](C(=O)NC(C)(C)Cn2nc(C(F)(F)F)cc2-c2cnn(C3CCCCO3)n2)N2CCc3c(-c4nc5cnn(C6CCCCO6)c5cc4C#N)ccc(F)c3C2=O)cn1] | 884.5 (M + H) |
| Prep 160 | (2R)-N-(1-(5-(2H-1,2,3- Triazol-4-yl)-3- (trifluoromethyl)-1H- pyrazol-1-yl)-2- methylpropan-2-yl)-2- (5-(6-cyano-1- (tetrahydro-2H-pyran-2- yl)-1H-indazol-5-yl)-8- fluoro-1-oxo-3,4- dihydroisoquinolin- 2(1H)-yl)-3-(1-methyl- 1H-pyrazol-4- yl)propanamide | 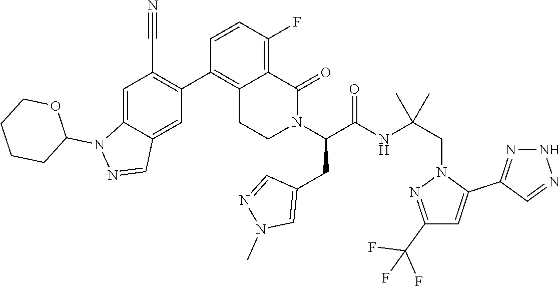 [SMILES: Cn1cc(C[C@H](C(=O)NC(C)(C)Cn2nc(C(F)(F)F)cc2-c2cn[nH]n2)N2CCc3c(-c4cc5cnn(C6CCCCO6)c5cc4C#N)ccc(F)c3C2=O)cn1] | 799.4 (M + H) |
| Prep 161 | (2R)-N-(1-(5-(1H-1,2,3- Triazol-5-yl)-3- (trifluoromethyl)-1H- pyrazol-1-yl)-2- methylpropan-2-yl)-3- (1-(difluoromethyl)-1H- pyrazol-4-yl)-2-(8- fluoro-5-(6-fluoro-1- (tetrahydro-2H-pyran-2- yl)-1H-pyrazolo[4,3- b]pyridin-5-yl)-1-oxo- 3,4-dihydroisoquinolin- 2(1H)-yl)propanamide | 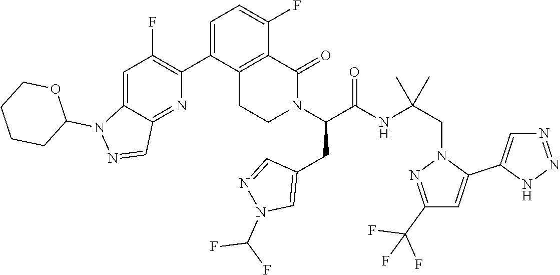 [SMILES: CC(C)(Cn1nc(C(F)(F)F)cc1-c1cnn[nH]1)NC(=O)[C@@H](Cc1cnn(C(F)F)c1)N1CCc2c(-c3nc4cnn(C5CCCCO5)c4cc3F)ccc(F)c2C1=O] | 829.3 (M + H) |
| Prep 162ª | (R)-2-(5-(2-Cyano-4-(2- methyl-2H- pyrazolo[3,4-b]pyridin- 5-yl)phenyl)-8-fluoro-1- oxo-3,4- dihydroisoquinolin- 2(1H)-yl)-N-(1-(5-(5- methoxy-1,2,4- thiadiazol-3-yl)-3- (trifluoromethyl)-1H- pyrazol-1-yl)-2- methylpropan-2-yl)-3- (1-methyl-1H-pyrazol- 4-yl)propanamide | 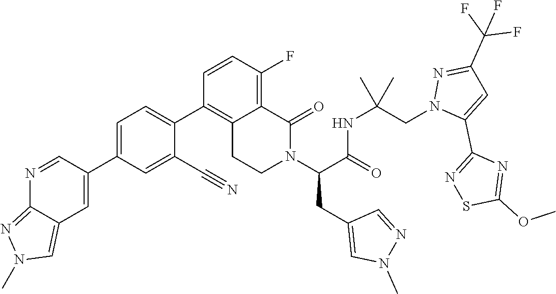 | 853.4 (M + H) |
| Prep 163 | (2R)-2-(5-(6-Cyano-1- (tetrahydro-2H-pyran-2- yl)-1H-indazol-5-yl)-8- fluoro-1-oxo-3,4- dihydroisoquinolin- 2(1H)-yl)-N-(1-(5-(5- methoxy-1,2,4- thiadiazol-3-yl)-3- (trifluoromethyl)-1H- pyrazol-1-yl)-2- methylpropan-2-yl)-3- (1-methyl-1H-pyrazol-4- yl)propanamide | 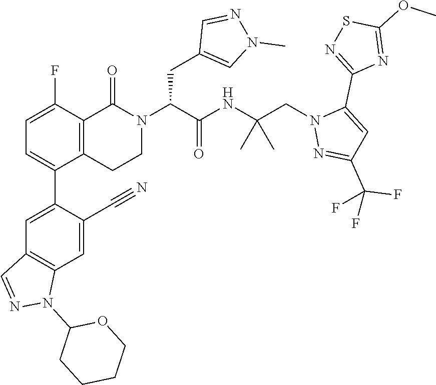 [SMILES: COc1nc(-c2cc(C(F)(F)F)nn2CC(C)(C)NC(=O)[C@@H](Cc2cnn(C)c2)N2CCc3c(-c4cc5cnn(C6CCCCO6)c5cc4C#N)ccc(F)c3C2=O)ns1] | 846.4 (M + H) |
| Prep 164 | (2R)-2-(5-(6-Cyano-1- (tetrahydro-2H-pyran-2- yl)-1H-pyrazolo[4,3- b]pyridin-5-yl)-8-fluoro- 1-oxo-3,4- dihydroisoquinolin- 2(1H)-yl)-N-(1-(5-(5- hydroxy-1,2,4- thiadiazol-3-yl)-3- (trifluoromethyl)-1H- pyrazol-1-yl)-2- methylpropan-2-yl)-3- (1-methyl-1H-pyrazol-4- yl)propanamide | 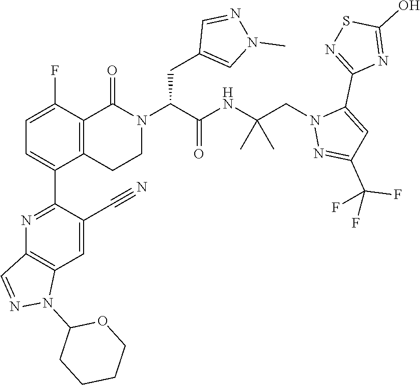 [SMILES: Cn1cc(C[C@H](C(=O)NC(C)(C)Cn2nc(C(F)(F)F)cc2-c2nsc(O)n2)N2CCc3c(-c4nc5cnn(C6CCCCO6)c5cc4C#N)ccc(F)c3C2=O)cn1] | 833.5 (M + H) |
| Prep 165 | Methyl (R)-2-(5-(6- cyano-1-methyl-1H- indazol-5-yl)-8-fluoro-1- oxo-3,4- dihydroisoquinolin- 2(1H)-yl)-3-(1-methyl- 1H-pyrazol-4- yl)propanoate | 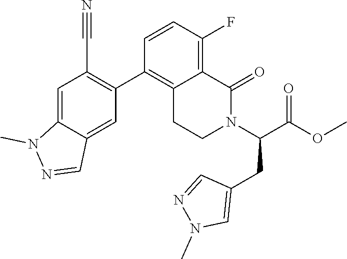 | 487.1 (M + H) |
| Prep 166^{b} | Methyl 3-((2R)-2-(5-(6- cyano-1-(tetrahydro-2H- pyran-2-yl)-1H-indazol- 5-yl)-8-fluoro-1-oxo- 3,4-dihydroisoquinolin- 2(1H)-yl)-3-(1-methyl- 1H-pyrazol-4- yl)propanamido)-3- methyl-2-(3- (trifluoromethyl)phenyl) butanoate | 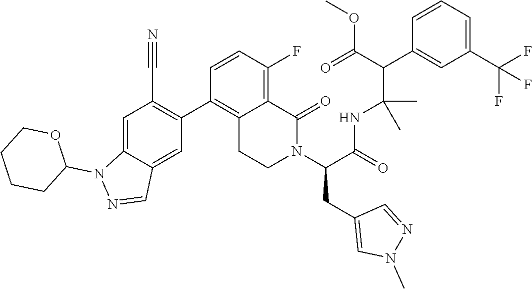 [SMILES: COC(=O)C(c1cccc(C(F)(F)F)c1)C(C)(C)NC(=O)[C@@H](Cc1cnn(C)c1)N1CCc2c(-c3cc4cnn(C5CCCCO5)c4cc3C#N)ccc(F)c2C1=O] | 800.2 (M + H) |
| Prep 167^{b} | Methyl 3-((2R)-2-(5-(6- cyano-1-(tetrahydro-2H- pyran-2-yl)-1H- pyrazolo[4,3-b]pyridin- 5-yl)-8-fluoro-1-oxo- 3,4-dihydroisoquinolin- 2(1H)-yl)-3-(1-methyl- 1H-pyrazol-4- yl)propanamido)-3- methyl-2-(3- (trifluoromethyl)phenyl) butanoate | 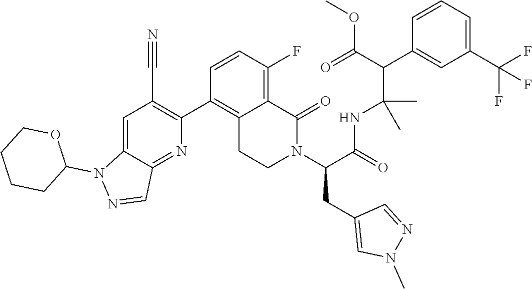 [SMILES: COC(=O)C(c1cccc(C(F)(F)F)c1)C(C)(C)NC(=O)[C@@H](Cc1cnn(C)c1)N1CCc2c(-c3nc4cnn(C5CCCCO5)c4cc3C#N)ccc(F)c2C1=O] | 801.2 (M + H) |
| Prep 168 | Methyl 3-((2R)-2-(5-(6- cyano-1-(tetrahydro-2H- pyran-2-yl)-1H-indazol- 5-yl)-8-fluoro-1-oxo- 3,4-dihydroisoquinolin- 2(1H)-yl)-3-(1-methyl- 1H-pyrazol-4- yl)propanamido)-2-(4- fluorophenyl)-3- methylbutanoate | 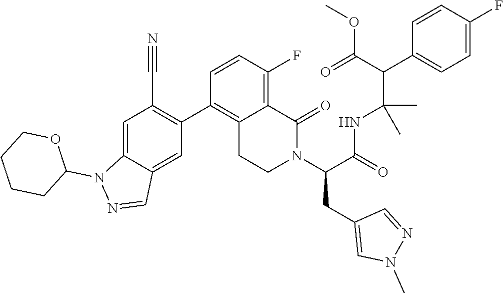 [SMILES: COC(=O)C(c1ccc(F)cc1)C(C)(C)NC(=O)[C@@H](Cc1cnn(C)c1)N1CCc2c(-c3cc4cnn(C5CCCCO5)c4cc3C#N)ccc(F)c2C1=O] | 750.5 (M + H) |
| Prep 169 | Methyl 3-((2R)-2-(5-(6- cyano-1-(tetrahydro-2H- pyran-2-yl)-1H- pyrazolo[4,3-b]pyridin- 5-yl)-8-fluoro-1-oxo- 3,4-dihydroisoquinolin- 2(1H)-yl)-3-(1-methyl- 1H-pyrazol-4- yl)propanamido)-2-(4- fluorophenyl)-3- methylbutanoate | 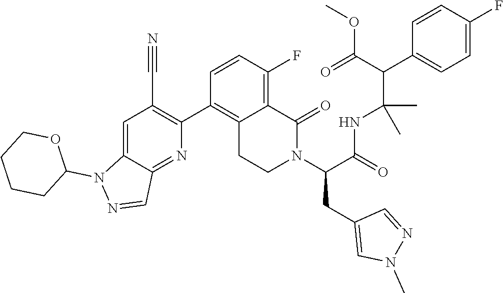 [SMILES: COC(=O)C(c1ccc(F)cc1)C(C)(C)NC(=O)[C@@H](Cc1cnn(C)c1)N1CCc2c(-c3nc4cnn(C5CCCCO5)c4cc3C#N)ccc(F)c2C1=O] | 751.5 (M + H) |
| Prep 170^{b} | Ethyl 3-((2R)-2-(5-(6- cyano-1-(tetrahydro-2H- pyran-2-yl)-1H- pyrazolo[4,3-b]pyridin- 5-yl)-8-fluoro-1-oxo- 3,4-dihydroisoquinolin- 2(1H)-yl)-3-(1-methyl- 1H-pyrazol-4- yl)propanamido)-3- methyl-2-(4- (trifluoromethyl)-1H- pyrazol-1-yl)butanoate | 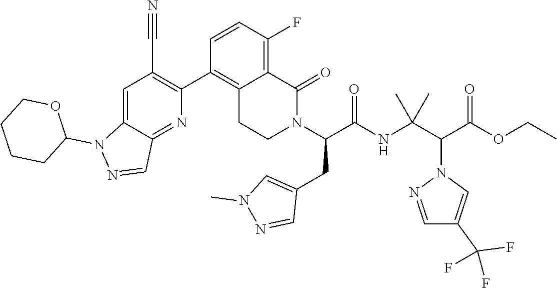 [SMILES: CCOC(=O)C(n1cc(C(F)(F)F)cn1)C(C)(C)NC(=O)[C@@H](Cc1cnn(C)c1)N1CCc2c(-c3nc4cnn(C5CCCCO5)c4cc3C#N)ccc(F)c2C1=O] | 805.2 (M + H) |
| Prep 171 | (2R)-2-(5-(6-Cyano-1- (tetrahydro-2H-pyran-2- yl)-1H-pyrazolo[4,3- b]pyridin-5-yl)-8-fluoro- 1-oxo-3,4- dihydroisoquinolin- 2(1H)-yl)-N-(1-(5- cyclopropyl-1H-pyrazol- 1-yl)-2-methylpropan-2- yl)-3-(1-methyl-1H- pyrazol-4- yl)propanamide | 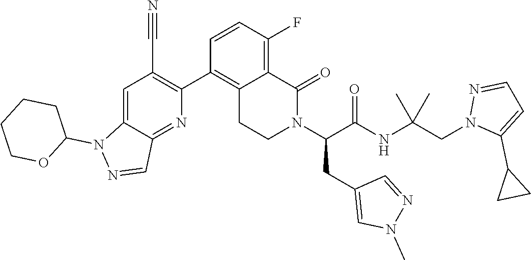 [SMILES: Cn1cc(C[C@H](C(=O)NC(C)(C)Cn2nccc2C2CC2)N2CCc3c(-c4nc5cnn(C6CCCCO6)c5cc4C#N)ccc(F)c3C2=O)cn1] | 705.2 (M + H) |

^{a}Pd(dppf)Cl₂, potassium carbonate, 1,4-dioxane/water, 80° C., 16 h
^{b}From the appropriate boronic ester, mesylate[(di(1-adamantyl)-n-butylphosphine)-2-(2′-amino-1,1′-biphenyl)]palladium(II), potassium carbonate, dioxane/water, 100° C., 12 h

### Preparation 172

### (2R)-2-(5-(6-Cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanoic acid

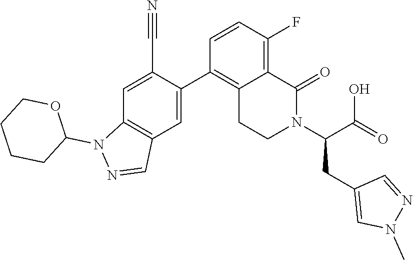 [SMILES: Cn1cc(C[C@H](C(=O)O)N2CCc3c(-c4cc5cnn(C6CCCCO6)c5cc4C#N)ccc(F)c3C2=O)cn1]

To a solution of (R)-(8-fluoro-2-(1-methoxy-3-(1-methyl-1H-pyrazol-4-yl)-1-oxopropan-2-yl)-1-oxo-1,2,3,4-tetrahydroisoquinolin-5-yl)boronic acid (500 mg, 87 wt %, 1.16 mmol) in 1,4-dioxane (5 mL) and water (0.5 mL) was added 5-chloro-1-(tetrahydro-2H-pyran-2-yl)-1H-indazole-6-carbonitrile (455 mg, 80% purity, 1.39 mmol), potassium carbonate (481 mg, 204 μL, 3.48 mmol) and mesylate[(di(1-adamantyl)-n-butylphosphine)-2-(2′-amino-1,1′-biphenyl)]palladium(II) (169 mg, 0.2 Eq, 232 μmol). The solution was stirred at 100° C. for 12 h under N₂. The solution was then cooled to 25° C. and treated with LiOH (2 mL, 1 M aq., 2 mmol). After stirring for 4 h at 25° C., the reaction mixture was diluted with H₂O (20 mL) and extracted with EtOAc (15 mL×3). The aqueous layer was separated and acidified to pH 4 with 1 N HCl, then the mixture was extracted with EtOAc (20 mL×3). The combined organic layers were washed with sat. aq. NaCl (10 mL×2), dried over Na₂SO₄, filtered and concentrated under reduced pressure. The reaction mixture was purified on silica gel (gradient 0-7% MeOH in DCM) to provide the title compound (440 mg, 64% yield, 91% purity) as a yellow solid. ES-MS m/z 543.2 (M+H).

### Preparation 173

### (R)-2-(5-(6-Cyano-1-methyl-1H-indazol-5-yl)-8-fluoro-1-oxo-3,4-dihydroisoquinolin-2 (1H)-yl)-3-(1-methyl-1H-pyrazol-4-yl)propanoic acid

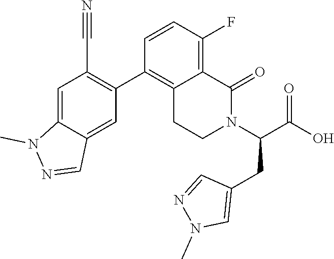 [SMILES: Cn1cc(C[C@H](C(=O)O)N2CCc3c(-c4cc5cnn(C)c5cc4C#N)ccc(F)c3C2=O)cn1]

To a solution of methyl (R)-2-(5-(6-cyano-1-methyl-1H-indazol-5-yl)-8-fluoro-1-oxo-3,4-dihydroisoquinolin-2(1H)-yl)-3-(1-methyl-1H-pyrazol-4-yl)propanoate (100 mg, 93% purity, 191 μmol) in THF (2 mL) was added 1 N LiOH (382 μL, 1 M aq., 382 μmol). The reaction mixture was stirred at 25° C. for 1 h, then adjusted to pH 4 with addition of 1 M HCl (aq), diluted with H₂O (4 mL) and extracted with EtOAc (10 mL×2). The combined organic layers were dried over Na₂SO₄, filtered and concentrated under reduced pressure to give the title compound (90 mg, 88% purity, 88% yield) as a yellow solid. ES-MS m/z 473.0 (M+H).

### Preparation 174

### (2R)-2-(5-(6-Cyano-1-(tetrahydro-2H-pyran-2-yl)-1H-indazol-5-yl)-8-fluoro-1-oxo-3,4-dihydroisoquinolin-2 (1H)-yl)-N-(1-(4-fluorophenyl)-2-methyl-1-(5-oxo-4,5-dihydro-1,2,4-thiadiazol-3-yl)propan-2-yl)-3-(1-methyl-1H-pyrazol-4-yl)propanamide

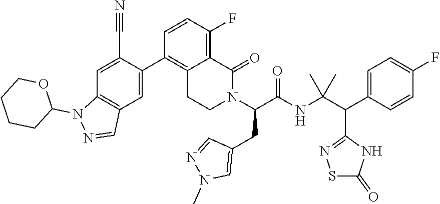 [SMILES: Cn1cc(C[C@H](C(=O)NC(C)(C)C(c2ccc(F)cc2)c2nsc(=O)[nH]2)N2CCc3c(-c4cc5cnn(C6CCCCO6)c5cc4C#N)ccc(F)c3C2=O)cn1]

To a mixture of (2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanoic acid (200 mg, 91% purity, 335 μmol), diethyl (4-oxo-1,2,3-benzotriazin-3-yl)phosphate (200 mg, 668 μmol) and NaHCO₃ (200 mg, 2.38 mmol) in DMF (5 mL) was added 3-(2-amino-1-(4-fluorophenyl)-2-methylpropyl)-1,2,4-thiadiazol-5 (4H)-one hydrochloride (130 mg, 70% purity, 300 μmol) at 0° C. The reaction mixture was stirred at 15° C. under N₂ for 2 h. Then 3-(2-amino-1-(4-fluorophenyl)-2-methylpropyl)-1,2,4-thiadiazol-5 (4H)-one hydrochloride (130 mg, 80% purity, 342 μmol) and diethyl (4-oxo-1,2,3-benzotriazin-3-yl)phosphate (150 mg, 501 μmol) were added and the reaction mixture was stirred at 15° C. for 16 h under N₂ atmosphere. The reaction mixture was then adjusted to pH 7 with 1 N HCl (aq), diluted with H₂O (10 mL), and extracted with EtOAc (15 mL×2). The combined organic layers were washed with sat. aq. NaCl (15 mL×2), dried over Na₂SO₄, filtered and concentrated under reduced pressure. The residue was purified by silica gel (gradient 0-80% EtOAc in DCM) to provide the title compound (180 mg, 52% yield, 76% purity) as a colorless solid. ES-MS m/z 792.1 (M+H).

### Preparation 175

### 3-((2R)-2-(5-(6-Cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanamido)-2-(4-fluorophenyl)-3-methylbutanoic acid

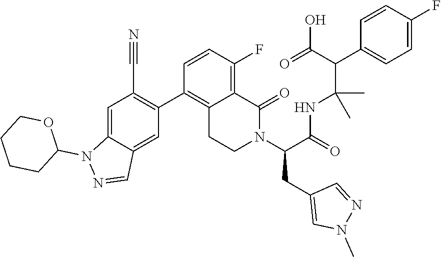 [SMILES: Cn1cc(C[C@H](C(=O)NC(C)(C)C(C(=O)O)c2ccc(F)cc2)N2CCc3c(-c4cc5cnn(C6CCCCO6)c5cc4C#N)ccc(F)c3C2=O)cn1]

To a solution of methyl 3-((2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanamido)-2-(4-fluorophenyl)-3-methylbutanoate (660 mg, 91 wt %, 801 μmol) in ACN (20 mL) and water (2 mL) was added 1,3,4,6,7,8-hexahydro-2H-pyrimido[1,2-a]pyrimidine (563 mg, 4.00 mmol). The reaction mixture was stirred at 60° C. for 3 h. The reaction mixture was then diluted with H₂O (20 mL) and acidified to pH 5 with 1 N HCl (aq.), then the mixture was extracted with EtOAc (30 mL×3). The combined organic layers were washed with sat. aq. NaCl (20 mL×2), dried over Na₂SO₄, filtered and concentrated under reduced pressure to provide the title compound (599 mg, 94% yield, 92% purity) as a brown solid. ES-MS m/z 736.4 (M+H).

The compounds bearing a carboxylic acid in the following table were prepared in a similar manner as described in Preparation 175 from the appropriate carboxylic ester. Reactants can be added in different orders or in differing equivalency, and purification methods were adjusted to suit the compounds. Such variances would be apparent to one skilled in the art.

TABLE 24

| Prepara- | | | ES-MS |
|---|---|---|---|
| tion | Chemical Name | Structure | m/z |
| 176 | 3-((2R)-2-(5-(6-Cyano-1- (tetrahydro-2H-pyran-2-yl)- 1H-pyrazolo[4,3-b]pyridin- 5-yl)-8-fluoro-1-oxo-3,4- dihydroisoquinolin-2(1H)- yl)-3-(1-methyl-1H-pyrazol- 4-yl)propanamido)-2-(4- fluorophenyl)-3- methylbutanoic acid | 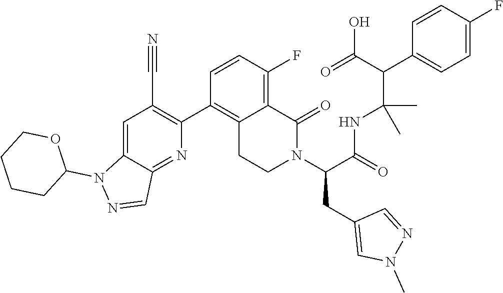 [SMILES: Cn1cc(C[C@H](C(=O)NC(C)(C)C(C(=O)O)c2ccc(F)cc2)N2CCc3c(-c4nc5cnn(C6CCCCO6)c5cc4C#N)ccc(F)c3C2=O)cn1] | 737.4 (M + H) |

### Example 162

### Methyl 3-((R)-2-(5-(6-cyano-1H-indazol-5-yl)-8-fluoro-1-oxo-3,4-dihydroisoquinolin-2 (1H)-yl)-3-(1-methyl-1H-pyrazol-4-yl)propanamido)-3-methyl-2-(3-(trifluoromethyl)phenyl)butanoate hydrochloride

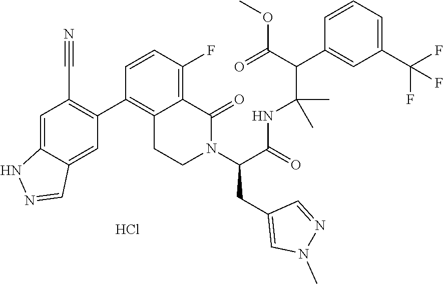 [SMILES: COC(=O)C(c1cccc(C(F)(F)F)c1)C(C)(C)NC(=O)[C@@H](Cc1cnn(C)c1)N1CCc2c(-c3cc4cn[nH]c4cc3C#N)ccc(F)c2C1=O ; Cl]

To a solution of methyl 3-((2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanamido)-3-methyl-2-(3-(trifluoromethyl)phenyl)butanoate (320 mg, 92 wt %, 368 μmol) in MeOH (5 mL) was added 2M hydrogen chloride in MeOH (3.67 g, 4.00 mL, 8.00 mmol) at 25° C. The mixture was stirred at 25° C. for 12 h. The mixture was then concentrated under reduced pressure to provide the title compound (292 mg, 95%, 90% Purity) as a yellow solid. ES-MS m/z 738.2 (M+Na).

The compounds in the following table were prepared in similar manner as described in Example 162 from the THP protected compound. Reactants can be added in different orders or in differing equivalency, and purification methods were adjusted to suit the compounds. Such variances would be apparent to one skilled in the art.

TABLE 25

| Ex- | | | ES-MS |
|---|---|---|---|
| ample | Chemical Name | Structure | m/z |
| 163 | Methyl 3-((R)-2-(5-(6-cyano- 1H-pyrazolo[4,3-b]pyridin-5- yl)-8-fluoro-1-oxo-3,4- dihydroisoquinolin-2(1H)-yl)- 3-(1-methyl-1H-pyrazol-4- yl)propanamido)-3-methyl-2- (3- (trifluoromethyl)phenyl) butanoate hydrochloride | 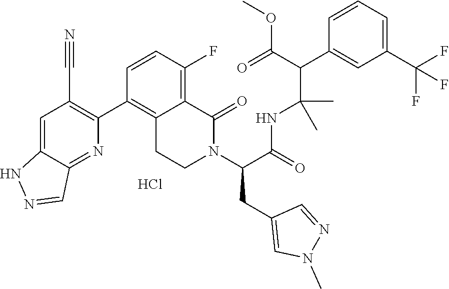 [SMILES: COC(=O)C(c1cccc(C(F)(F)F)c1)C(C)(C)NC(=O)[C@@H](Cc1cnn(C)c1)N1CCc2c(-c3nc4cn[nH]c4cc3C#N)ccc(F)c2C1=O ; Cl] | 739.1 (M + Na) |
| 164 | 3-((R)-2-(5-(6-Cyano-1H- indazol-5-yl)-8-fluoro-1-oxo- 3,4-dihydroisoquinolin-2(1H)- yl)-3-(1-methyl-1H-pyrazol-4- yl)propanamido)-2-(4- fluorophenyl)-3- methylbutanoic acid | 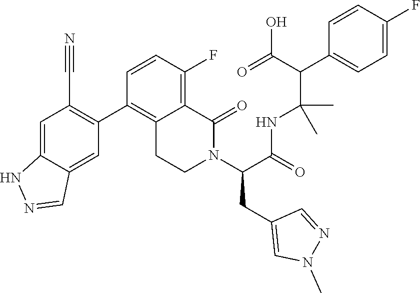 [SMILES: Cn1cc(C[C@H](C(=O)NC(C)(C)C(C(=O)O)c2ccc(F)cc2)N2CCc3c(-c4cc5cn[nH]c5cc4C#N)ccc(F)c3C2=O)cn1] | 652.8 (M + H) |
| 165 | 3-((R)-2-(5-(6-Cyano-1H- pyrazolo[4,3-b]pyridin-5-yl)-8- fluoro-1-oxo-3,4- dihydroisoquinolin-2(1H)-yl)- 3-(1-methyl-1H-pyrazol-4- yl)propanamido)-2-(4- fluorophenyl)-3- methylbutanoic acid | 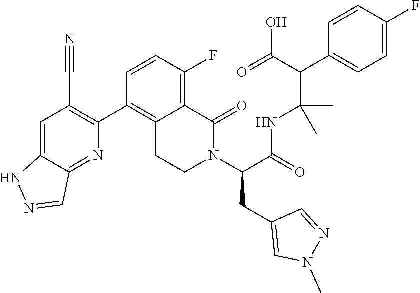 [SMILES: Cn1cc(C[C@H](C(=O)NC(C)(C)C(C(=O)O)c2ccc(F)cc2)N2CCc3c(-c4nc5cn[nH]c5cc4C#N)ccc(F)c3C2=O)cn1] | 653.8 (M + H) |

The compounds bearing a carboxylic acid in the following table were prepared in a similar manner as described in Preparation 175 from the appropriate carboxylic ester. Reactants can be added in different orders or in differing equivalency, and purification methods were adjusted to suit the compounds. Such variances would be apparent to one skilled in the art.

TABLE 26

| | | | ES-MS |
|---|---|---|---|
| Example | Chemical Name | Structure | m/z |
| 166 | 3-((R)-2-(5-(6-Cyano-1H- indazol-5-yl)-8-fluoro-1- oxo-3,4-dihydroisoquinolin- 2(1H)-yl)-3-(1-methyl-1H- pyrazol-4-yl)propanamido)- 3-methyl-2-(3- (trifluoromethyl)phenyl) butanoic acid | 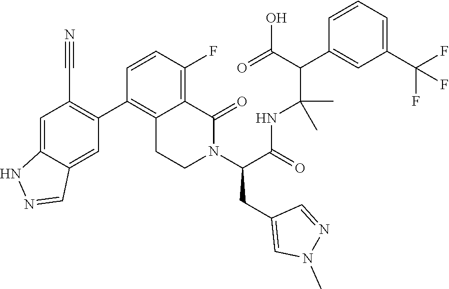 [SMILES: Cn1cc(C[C@H](C(=O)NC(C)(C)C(C(=O)O)c2cccc(C(F)(F)F)c2)N2CCc3c(-c4cc5cn[nH]c5cc4C#N)ccc(F)c3C2=O)cn1] | 702.2 (M + H) |
| 167 | 3-((R)-2-(5-(6-Cyano-1H- pyrazolo[4,3-b]pyridin-5- yl)-8-fluoro-1-oxo-3,4- dihydroisoquinolin-2(1H)- yl)-3-(1-methyl-1H-pyrazol- 4-yl)propanamido)-3- methyl-2-(3- (trifluoromethyl)phenyl) butanoic acid | 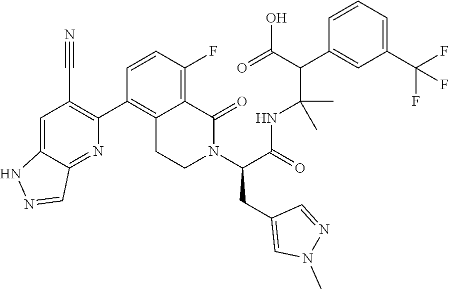 [SMILES: Cn1cc(C[C@H](C(=O)NC(C)(C)C(C(=O)O)c2cccc(C(F)(F)F)c2)N2CCc3c(-c4nc5cn[nH]c5cc4C#N)ccc(F)c3C2=O)cn1] | 703.8 (M + H) |

### Preparation 183

### 2-Morpholinoethyl 3-((2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanamido)-2-(4-fluorophenyl)-3-methylbutanoate

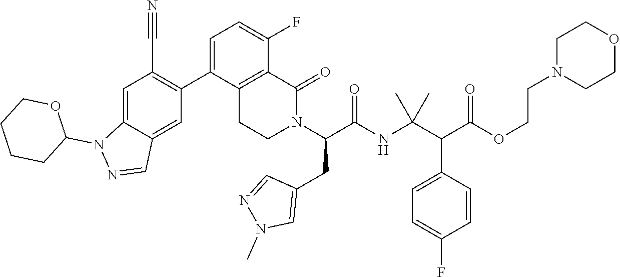 [SMILES: Cn1cc(C[C@H](C(=O)NC(C)(C)C(C(=O)OCCN2CCOCC2)c2ccc(F)cc2)N2CCc3c(-c4cc5cnn(C6CCCCO6)c5cc4C#N)ccc(F)c3C2=O)cn1]

A mixture of 3-((2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanamido)-2-(4-fluorophenyl)-3-methylbutanoic acid (150.0 mg, 187.6 μmol, 92% purity), DCC (78.2 mg, 375 μmol), and 1H-benzo[d][1,2,3]triazol-1-ol (51.2 mg, 375 μmol) in THF (5 mL) was stirred at 20° C. for 30 min. Then, 2-morpholinoethan-1-ol (62.1 mg, 469 μmol) was added and the mixture stirred at 20° C. for 16 h. The mixture was diluted with water (50 mL) and extracted with EtOAc (50 mL×3). The combined organic layers were washed with sat. aq. NaCl (50 mL×2), dried over Na₂SO₄, filtered, and the filtrate concentrated under reduced pressure. The residue was purified on silica gel, eluting with 0-5% MeOH in DCM, to obtain the title compound (96.3 mg, 57%) as a white solid. ES-MS m/z 849.5 (M+H).

### Preparation 184

### 1-(Propionyloxy)ethyl 3-((2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanamido)-2-(4-fluorophenyl)-3-methylbutanoate

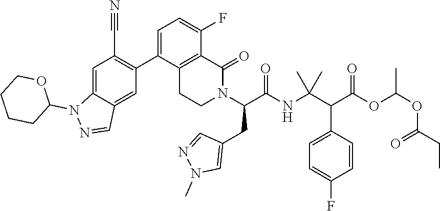 [SMILES: CCC(=O)OC(C)OC(=O)C(c1ccc(F)cc1)C(C)(C)NC(=O)[C@@H](Cc1cnn(C)c1)N1CCc2c(-c3cc4cnn(C5CCCCO5)c4cc3C#N)ccc(F)c2C1=O]

To a solution of 1-chloroethyl propionate (200 mg, 1.24 mmol) and 3-((2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-methyl-1H-pyrazol-4-yl)propanamido)-2-(4-fluorophenyl)-3-methylbutanoic acid (100 mg, 125 μmol) in acetone (2 mL) was added K₂CO₃ (50 mg, 0.36 mmol) and potassium iodide (50 mg, 0.30 mmol). The mixture was stirred at 40° C. for 16 h. The reaction mixture was quenched with water (10 mL) and extracted with EtOAc (10 mL×2). The combined organic layers were washed with sat. aq. NaCl (10 mL×2), dried over sodium sulfate, filtered, and the filtrate concentrated under reduced pressure. The residue was purified on prep-TLC, eluting with 10:1 DCM/MeOH (R_{f}=0.6), to give the title compound (68 mg, 61% yield, 93% purity) as a yellow solid. ES-MS m/z 858.3 (M+Na).

### Preparation 185

### 6-Bromo-1-methyl-1H-pyrazolo[4,3-b]pyridine

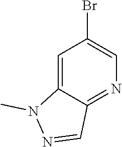

The following procedure was performed in 14 reactions carried out in parallel on the scale given herein. A mixture of 6-bromo-1H-pyrazolo[4,3-b]pyridine (500 g, 2.52 mol) and K₂CO₃ (523 g, 3.79 mol) in DMF (3.50 L) was degassed and purged with N₂ 3 times. To the mixture was slowly added iodomethane (430 g, 3.03 mol) at 25-35° C. The mixture was stirred at 25° C. for 6 h under N₂ atmosphere.

Every two reactions were combined for workup. The reaction mixtures were quenched by H₂O (21.0 L) at 0-5° C. and extracted with EtOAc (1.00 L×6). The combined organic layers were washed with sat. aq. NaCl (6.00 L×2), then the organic layers concentrated under reduced pressure.

All fourteen reactions were combined for purification. The products obtained from aqueous workup were purified on silica gel, eluting with 5-11% EtOAc in petroleum ether and then triturated with MTBE (3.50 L) at 25° C. for 1 h. The solid was collected by filtration and dried under reduced pressure to give the title compound (3.51 kg, 47%) as a yellow solid. ES-MS m/z 212 (M+H).

### Preparation 186

### 1-Methyl-1H-pyrazolo[4,3-b]pyridine-6-carbonitrile

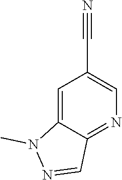

The following procedure was performed in 4 reactions carried out in parallel on the scale given herein. A mixture of 6-bromo-1-methyl-1H-pyrazolo[4,3-b]pyridine (104 g, 490 mmol), Pd(PPh₃)₄ (113 g, 98.0 mmol), and Zn(CN)₂ (85.5 g, 728 mmol) in DMF (1.04 L) was degassed and purged with N₂ 3 times. The mixture was stirred at 120° C. for 12 h under N₂ atmosphere.

The four reactions were combined for workup. The combined reaction mixtures were filtered, and water (12.0 L) was added to the filtrate, and the mixture was extracted with EtOAc (3.20 L×5). The combined organic layers were washed with sat. aq. NaCl (1.00 L) and the organic layers were concentrated under reduced pressure. The residue was purified on silica gel, eluting with 0-100% EtOAc in petroleum ether to afford the title compound (300 g, 89% yield) as a white solid. ES-MS m/z 159 (M+H).

### Preparation 187

### 6-Cyano-1-methyl-1H-pyrazolo[4,3-b]pyridine 4-oxide

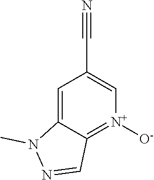

The following procedure was performed in 3 reactions carried out in parallel on the scale given herein. A mixture of 1-methyl-1H-pyrazolo[4,3-b]pyridine-6-carbonitrile (100 g, 632 mmol), methyltrioxorhenium (15.8 g, 63.2 mmol), and H₂O₂ (30 wt % aq. solution, 361 g, 3.19 mol, 306 mL) in DCM (1.00 L) was degassed and purged with N₂ 3 times. The mixture was stirred at 45° C. for 48 h under N₂ atmosphere.

The three reactions were combined for workup. MnO₂ (10 wt %, 30 g) and H₂O (900 mL) were added into the combined reaction mixtures at 45° C. under N₂ and the resulting mixture was stirred for 30 min at 40-45° C. The mixture was filtered, and the filter cake was washed with DCM (600 mL) to provide a mixture of the title compound and MnO₂ (300 g) as a gray solid. TLC (EtOAc) R_{f}=0.3. The mixture was used without further purification.

### Preparation 188

### 5-Chloro-1-methyl-1H-pyrazolo[4,3-b]pyridine-6-carbonitrile

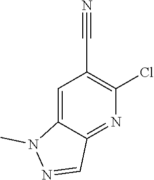

Three reactions were carried out in parallel on the scale given herein. To a solution of 6-cyano-1-methyl-1H-pyrazolo[4,3-b]pyridine 4-oxide (100 g, 574 mmol) and DIEA (111 g, 861 mmol, 150 mL) in DCM (1.00 L) was added oxalyl chloride (87.4 g, 689 mmol) dropwise at 0° C. over 1 h. The resulting mixture was stirred at 0-20° C. for 6 h.

The three reactions were combined for workup. The combined reaction mixtures were quenched by water (900 mL) and extracted with DCM (600 mL×2). The combined organic layers were washed with sat. aq. NaCl (900 mL) and the organic phase was concentrated under reduced pressure to give the residue. The residue was purified on silica gel eluting with 25%-100% EtOAc in petroleum ether to obtain the title compound (200 g, 60% yield) as an off-white solid. ES-MS m/z 193 (M+H).

### Preparation 189

### 3,5-Dichloro-1-methyl-1H-pyrazolo[4,3-b]pyridine-6-carbonitrile

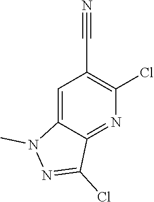

A mixture of 5-chloro-1-methyl-1H-pyrazolo[4,3-b]pyridine-6-carbonitrile (1.0 g, 5.2 mmol), NCS (0.83 g, 6.2 mmol), and DMF (10 mL) under nitrogen atmosphere was heated to 50° C. for 25 h. Additional NCS (350 mg, 2.6 mmol) was added and the mixture was heated to 50° C. for 23 h. The mixture was then diluted with water (20 mL) and heated to reflux for 1 min, then cooled to RT and filtered. The resulting solid was dissolved in MeOH (40 mL) at reflux then cooled to RT. Vacuum filtration gave the title compound (800 mg, 68% yield) as a white solid. ES-MS m/z 227 (M+H).

### Preparation 190

### 5-Chloro-3-fluoro-1-methyl-1H-pyrazolo[4,3-b]pyridine-6-carbonitrile

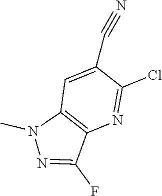

A mixture of 5-chloro-1-methyl-1H-pyrazolo[4,3-b]pyridine-6-carbonitrile (7.70 g, 40.0 mmol), ACN (72.0 mL), and 1-chloromethyl-4-fluoro-1,4-diazoniabicyclo[2.2.2]octane bis(tetrafluoroborate) (28.3 g, 79.9 mmol) was heated to 100° C. for 20 h in a 500 mL pressure vessel. The mixture was diluted with water (500 mL) and sat. aq. K₂CO₃ to PH ˜8 to form a precipitate. The solids were collected via vacuum filtration, then the solids and filtrate were combined and diluted with EtOAc. The resulting solution was washed with sat. aq. NaCl, dried over MgSO₄, filtered, concentrated. The residue was purified by reverse phase HPLC using a gradient of 18 to 45% ACN in aq. NH₄HCO₃, and the product was triturated with DCM and cyclohexane to provide the title compound (2.24 g, 27% yield) as a pale yellow solid. ES-MS m/z 211 (M+H).

### Preparation 191

### 5-Bromo-6-fluoro-1-methyl-1H-pyrazolo[4,3-b]pyridine

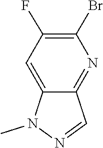

A mixture of 5-bromo-6-fluoro-1H-pyrazolo[4,3-b]pyridine (1.95 g, 80 wt %, 7.22 mmol) in DMF (20 mL) was purged with N₂ three times, then 60% NaH in mineral oil (347 mg, 60 wt %, 8.67 mmol) was added to the mixture at 0° C. The mixture was stirred at 0° C. for 30 min under N₂, then iodomethane (1.55 g, 708 μL, 10.8 mmol) was added. The mixture was stirred at 0° C. for 1 h under N₂, then it was poured into H₂O (80 mL) and extracted with EtOAc (60 mL×3). The combined organic layers were washed with sat. aq. NaCl (60 mL×3), dried over anhydrous Na₂SO₄, filtered, and concentrated under reduced pressure. The residue was purified on silica gel, eluting with 6-7% EtOAc in 4:1 hexanes:DCM to obtain the title compound (746 mg, 44% yield) as a yellow solid. ES-MS m/z 230, 232 (M+H).

### Preparation 192

### 5-Bromo-3-chloro-6-fluoro-1-methyl-1H-pyrazolo[4,3-b]pyridine

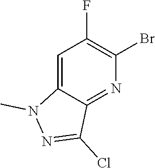

To a solution of 5-bromo-6-fluoro-1-methyl-1H-pyrazolo[4,3-b]pyridine (100 mg, 426 μmol) in ACN (1 mL) was added NCS (60 mg, 1.1 Eq, 0.45 mmol). The reaction mixture was stirred at 50° C. for 3 h, then diluted with water (5 mL) and extracted with EtOAc (5 mL×2). The combined organic layers were dried over Na₂SO₄, filtered, and concentrated under reduced pressure. The residue was purified by prep-TLC (1:3 EtOAc/hexanes, R_{f}=0.6) to give the title compound (104 mg, 90% yield) as a white solid. ES-MS m/z 264, 266 (M+H).

### Preparation 193

### 5-Bromo-3,6-difluoro-1-methyl-1H-pyrazolo[4,3-b]pyridine

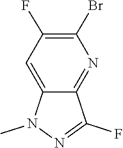

Three reactions were performed in parallel on the scale given herein. A mixture of 5-bromo-6-fluoro-1-methyl-1H-pyrazolo[4,3-b]pyridine (900 mg, 3.87 mmol) and 1-chloromethyl-4-fluoro-1,4-diazoniabicyclo[2.2.2]octane bis(tetrafluoroborate) (4.16 g, 11.6 mmol) in ACN (12 mL) was degassed with N₂. The mixture was stirred at 100° C. for 16 h. The mixture was diluted with H₂O (80 mL) and extracted with EtOAc (60 mL×3). The combined organic layers were dried over Na₂SO₄, filtered, and concentrated under reduced pressure.

Residues from the three reactions were combined and purified on silica gel, eluting with 0-3% EtOAc in 4:1 hexanes:DCM, then re-purified by reverse phase prep-HPLC [column: Welch Xtimate C₁₈; mobile phase: gradient of 20%-60% ACN in water (with 0.225% FA)] to give the title compound (1.41 g, 49% yield) as a white solid. ES-MS m/z 248 (M+H).

### Preparation 194

### 5-Chloro-3,6-difluoro-1-methyl-1H-pyrazolo[4,3-b]pyridine

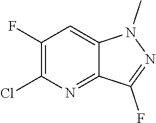

A mixture of 5-chloro-6-fluoro-1-methyl-1H-pyrazolo[4,3-b]pyridine (1.60 g, 8.62 mmol), ACN (7.18 mL), and 1-chloromethyl-4-fluoro-1,4-diazoniabicyclo[2.2.2]octane bis(tetrafluoroborate) (15.3 g, 43.2 mmol) was heated to 70° C. under N₂ for 20 h. The mixture was then heated to 90° C. for an additional 6 h. The mixture was diluted with water and extracted with DCM. The organic layer was concentrated under reduced pressure. The residue was purified on silica gel, eluting with 10-20% EtOAc in cyclohexane to provide a white solid. Additional purification was done by reverse phase flash chromatography, using an ammonium bicarbonate modified water/ACN solvent system (30-45%) on a C-18 column to provide a suspension. Solids were collected via vacuum filtration to yield the title compound (600 mg, 34% yield) as a white solid. ES-MS m/z 204 (M+H).

### Preparation 195

### tert-Butyl (R)-(3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)carbamate

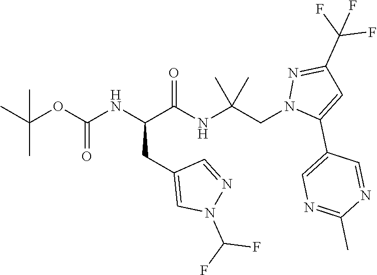 [SMILES: Cc1ncc(-c2cc(C(F)(F)F)nn2CC(C)(C)NC(=O)[C@@H](Cc2cnn(C(F)F)c2)NC(=O)OC(C)(C)C)cn1]

A mixture of tert-butyl (R)-(1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate (45.0 g, 78.5 mmol), 2-methyl-5-(4,4,5,5-tetramethyl-1,3,2-dioxaborolan-2-yl)pyrimidine (24.2 g, 110 mmol), K₂CO₃ (32.5 g, 235 mmol), and Pd(dppf)Cl₂ (5.74 g, 7.84 mmol) in nitrogen-sparged 1,4-dioxane (314 mL) and nitrogen-sparged water (78.5 mL) was stirred at 90° C. under an atmosphere of nitrogen for 2 h. Then, the mixture was partially concentrated to a volume of 150 mL under reduced pressure and partitioned between EtOAc (500 mL) and water (500 mL). Saturated aq. NaCl (50 mL) was added, and then the organic layer was removed, dried over Na₂SO₄, filtered, and the filtrate concentrated under reduced pressure. The residue was purified on silica gel, eluting with 0-10% of MeOH in DCM, to obtain the title compound (48.0 g, 90% purity, 94% yield) as a dark foamy solid. ES-MS m/z 531 (M+H-tBu).

### Preparation 196

### (R)-2-Amino-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide dihydrochloride

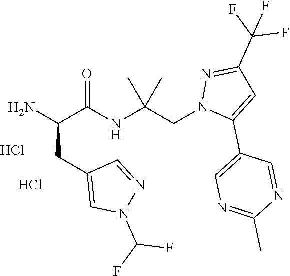 [SMILES: Cl ; Cl ; Cc1ncc(-c2cc(C(F)(F)F)nn2CC(C)(C)NC(=O)[C@H](N)Cc2cnn(C(F)F)c2)cn1]

A solution of tert-butyl (R)-(3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)carbamate (48.0 g, 73.6 mmol, 90% purity) in 1,4-dioxane (245 mL) was treated with HCl (125 mL of a 4 M solution in 1,4-dioxane, 500 mmol) and stirred at 35° C. for 3 h under an atmosphere of nitrogen. The mixture was then concentrated to dryness under reduced pressure to obtain the title compound (44.0 g, 95% purity, 100% yield) as a red powder. ES-MS m/z 487 (M+H).

### Preparation 197

### Methyl (R)-2-amino-3-(1-(difluoromethyl)-1H-pyrazol-4-yl)propanoate hydrochloride

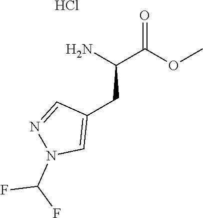

(R)-2-((tert-butoxycarbonyl)amino)-3-(1-(difluoromethyl)-1H-pyrazol-4-yl)propanoic acid (39.9 g, 131 mmol) in 2 M HCl in MeOH (392 mL, 784 mmol) was stirred at 25° C. for 16 h. Then, the reaction mixture was concentrated under reduced pressure. The resulting residue was re-dissolved in a mixture of THF (100 mL) and 1,4-dioxane (100 mL) and concentrated under reduced pressure to obtain the title compound (35.7 g, 90% purity, 96% yield) as a white solid. ES-MS m/z 220 (M+H).

### Preparation 198

### (R)-3-Bromo-2-(2-chloroethyl)-N-(3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)-6-fluorobenzamide

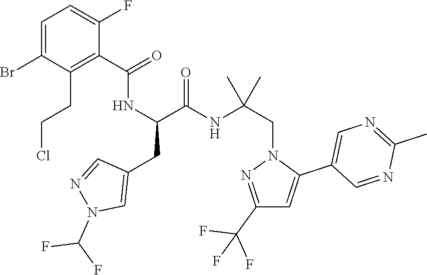 [SMILES: Cc1ncc(-c2cc(C(F)(F)F)nn2CC(C)(C)NC(=O)[C@@H](Cc2cnn(C(F)F)c2)NC(=O)c2c(F)ccc(Br)c2CCCl)cn1]

A mixture of (R)-2-amino-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide dihydrochloride (44.0 g, 78.7 mmol) in DCM (393 mL) was cooled in an ice-water bath under nitrogen and DIEA (54.2 mL, 315 mmol) was added, followed by dropwise addition of a solution of 3-bromo-2-(2-chloroethyl)-6-fluorobenzoyl chloride (26.0 g, 86.7 mmol) in DCM (100 mL). The ice bath was removed, and the resulting mixture was stirred at RT for 30 min, then diluted with water (500 mL) and adjusted to pH 3 with 1 N aq. HCl. The layers were separated, and the organic layer was washed with 300 mL water and 300 mL sat. aq. NaHCO₃, dried over Na₂SO₄, and concentrated under reduced pressure to provide the title compound as an oil (64.9 g, 99% yield, 90% purity). ES-MS m/z 749, 751 (M+H).

### Preparation 199

### (R)-2-(5-Bromo-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide

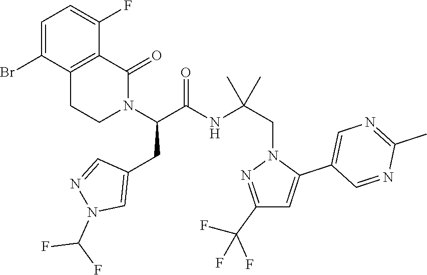 [SMILES: Cc1ncc(-c2cc(C(F)(F)F)nn2CC(C)(C)NC(=O)[C@@H](Cc2cnn(C(F)F)c2)N2CCc3c(Br)ccc(F)c3C2=O)cn1]

A mixture of (R)-3-bromo-2-(2-chloroethyl)-N-(3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)-6-fluorobenzamide (64.9 g, 7.9 mmol, 90% purity), ACN (195 mL), and Cs₂CO₃ (63.4 g, 195 mmol) under nitrogen was stirred at 30° C. for 21 h, concentrated under reduced pressure to 50 mL total volume, diluted with EtOAc (700 mL) and washed with water (1 L). The organics were then shaken in a separatory funnel with 1 N HCl aq (1 L) for 2 min, and the water layer was removed. The organic layer was washed with half-saturated aq. NaHCO₃, dried over Na₂SO₄, and concentrated under reduced pressure. The residue was purified by silica gel, eluting with 40-80% EtOAc in cyclohexane to provide the title compound as a tan foam (49.9 g, 90%). ES-MS m/z 713, 715 (M+H).

### Preparation 200

### (R)-3-(1-(Difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)propanamide

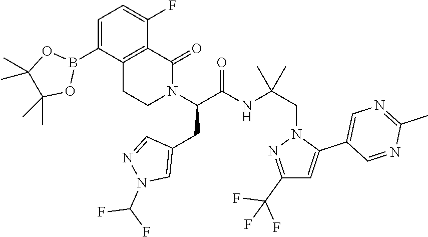

In a N₂-filled glovebox, (R)-2-(5-bromo-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide (600 mg, 799 μmol, 95% purity), 4,4,4′,4′,5,5,5′,5′-octamethyl-2,2′-bi (1,3,2-dioxaborolane) (1.62 g, 6.39 mmol), potassium acetate (235 mg, 2.40 mmol) and (2-dicyclohexylphosphino-2′,6′-dimethoxybiphenyl)[2-(2′-amino-1,1′-biphenyl)]palladium(II) methanesulfonate (69.9 mg, 79.9 μmol) were added to a dried reaction vial, followed by DMF (20 mL). The vial was sealed, removed from the glovebox, and stirred at 100° C. for 16 h. The reaction mixture was diluted with water (50 mL) and extracted with EtOAc (30 mL×2). The combined organic layer was washed with water (30 mL×2), dried over anhydrous Na₂SO₄, and concentrated under reduced pressure. The residue was purified on silica gel with a gradient of 0-100% EtOAc in hexane to provide the title compound (486 mg, 76%, 95% purity) as a brown oil. ES-MS m/z 761 (M+H).

### Preparation 201

### Methyl (R)-2-(3-bromo-2-(2-chloroethyl)-6-fluorobenzamido)-3-(1-(difluoromethyl)-1H-pyrazol-4-yl)propanoate

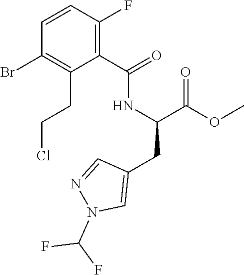 [SMILES: COC(=O)[C@@H](Cc1cnn(C(F)F)c1)NC(=O)c1c(F)ccc(Br)c1CCCl]

To a mixture of methyl (R)-2-amino-3-(1-(difluoromethyl)-1H-pyrazol-4-yl)propanoate hydrochloride (29 g, 0.10 mol, 90% purity) and TEA (0.14 L, 1.0 mol) in THF (260 mL) was added a solution of 3-bromo-2-(2-chloroethyl)-6-fluorobenzoyl chloride (53 g, 0.14 mol, 81% purity) in THF (150 mL) at 0° C. The reaction mixture was stirred at 0° C. for 4 h, diluted with water (400 mL) and extracted with EtOAc (300 mL×2). The combined organic layers were washed with sat. aq. NaCl (200 mL×2), dried over anhydrous Na₂SO₄, filtered, and concentrated under reduced pressure. The residue was purified on silica gel with a gradient of 0-25% EtOAc in hexanes to provide the title compound (47.48 g, 92%, 96% purity) as a yellow solid. ES-MS m/z 482 (M+H).

### Preparation 202

### Methyl (R)-2-(5-bromo-8-fluoro-1-oxo-3,4-dihydroisoquinolin-2(1H)-yl)-3-(1-(difluoromethyl)-1H-pyrazol-4-yl)propanoate

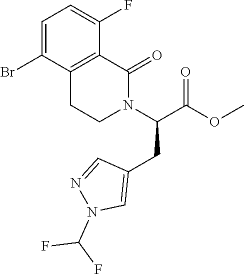 [SMILES: COC(=O)[C@@H](Cc1cnn(C(F)F)c1)N1CCc2c(Br)ccc(F)c2C1=O]

A mixture of methyl (R)-2-(3-bromo-2-(2-chloroethyl)-6-fluorobenzamido)-3-(1-(difluoromethyl)-1H-pyrazol-4-yl)propanoate (47.48 g, 94.43 mmol, 96% purity), water (0.20 mL, 11 mmol) and cesium carbonate (36.5 g, 112 mmol) in ACN (500 mL) was stirred at 25° C. for 2 h, filtered, and the filter cake was washed with EtOAc (25 mL×3). The filtrate was concentrated to remove most of the solvent, diluted with H₂O (100 mL) and extracted with EtOAc (100 mL×2). The combined organic layers were washed with sat. aq. NaCl (100 mL×2), dried over anhydrous Na₂SO₄, filtered, and concentrated under reduced pressure. The residue was purified on silica gel with a gradient of 0-40% EtOAc in hexanes to provide the title compound (42.45 g, 96%, 95% purity) as a yellow oil. ES-MS m/z 446, 448 (M+H).

### Preparation 203

### (R)-(2-(3-(1-(Difluoromethyl)-1H-pyrazol-4-yl)-1-methoxy-1-oxopropan-2-yl)-8-fluoro-1-oxo-1,2,3,4-tetrahydroisoquinolin-5-yl)boronic acid

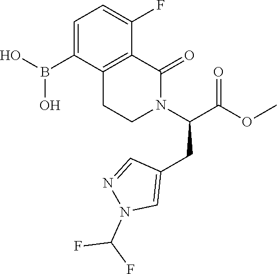 [SMILES: COC(=O)[C@@H](Cc1cnn(C(F)F)c1)N1CCc2c(B(O)O)ccc(F)c2C1=O]

In a N₂-filled glovebox, a solution of methyl (R)-2-(5-bromo-8-fluoro-1-oxo-3,4-dihydroisoquinolin-2(1H)-yl)-3-(1-(difluoromethyl)-1H-pyrazol-4-yl)propanoate (11.8 g, 25.1 mmol, 95% purity) in MeOH (210 mL) was treated with hypodiboric acid (11.3 g, 126 mmol), a solution of XPhos Pd G3 (2.13 g, 2.51 mmol) in DCE (30 mL), and potassium acetate (7.40 g, 75.4 mmol). The reaction mixture was removed from the glovebox and stirred at 40° C. for 16 h under N₂, filtered, adjusted pH 7 with formic acid, and concentrated. The residue was purified on silica gel with a gradient of 0-4% MeOH+0.5% formic acid in DCM to provide the title compound (11.9 g, 98%, 85% purity) as a brown oil. ES-MS m/z 412 (M+H).

### Preparation 204

### Methyl (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoate

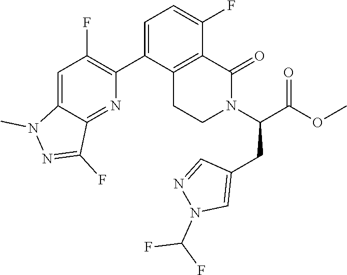

A N₂-degassed mixture of (R)-(2-(3-(1-(difluoromethyl)-1H-pyrazol-4-yl)-1-methoxy-1-oxopropan-2-yl)-8-fluoro-1-oxo-1,2,3,4-tetrahydroisoquinolin-5-yl)boronic acid (2.4 g, 5.0 mmol, 85% purity), 5-bromo-3,6-difluoro-1-methyl-1H-pyrazolo[4,3-b]pyridine (1.41 g, 5.63 mmol, 99% purity), sodium carbonate (1.6 g, 15 mmol) and XPhos Pd G3 (0.84 g, 0.99 mmol) in 1,4-dioxane (80 mL) and water (8 mL) was stirred at 90° C. for 1 h under N₂, filtered, and concentrated. The residue was purified on silica gel eluting with a gradient of 0-5% MeOH in DCM to provide the title compound (1.83 g, 68%, 98% purity) as a yellow solid. ES-MS m/z 535 (M+H).

### Preparation 205

### (R)-2-(5-(3,6-Difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid

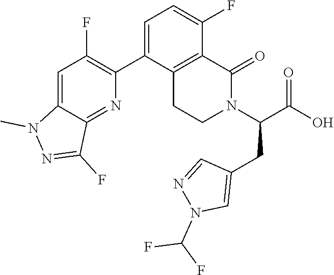 [SMILES: Cn1nc(F)c2nc(-c3ccc(F)c4c3CCN([C@H](Cc3cnn(C(F)F)c3)C(=O)O)C4=O)c(F)cc21]

To a mixture of methyl (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoate (1.83 g, 3.36 mmol, 98% purity) in tert-butanol (50 mL) and water (25 mL) was added 1 N aq. LiOH (10.5 mL, 10.5 mmol) at 0° C. The reaction mixture was stirred at 0° C. for 3 h, adjusted to pH 3 with 1 N aq. HCl, diluted with H₂O (50 mL), and extracted with EtOAc (50 mL×2). The combined organic layers were dried over anhydrous Na₂SO₄, filtered, and concentrated under reduced pressure to provide the title compound (1.9 g, 98%, 90% purity) as a yellow solid. ES-MS m/z 521 (M+H).

### Preparation 206

### Methyl 3-(2-(diphenylmethylene)hydrazineyl)-5-fluoropicolinate

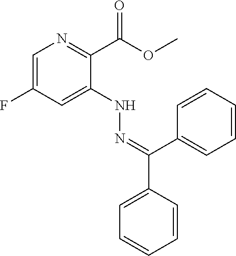 [SMILES: COC(=O)c1ncc(F)cc1NN=C(c1ccccc1)c1ccccc1]

A 250-mL, 3-necked RBF equipped with a thermocouple, nitrogen inlet and reflux condenser was charged with (9,9-dimethyl-9H-xanthene-4,5-diyl)bis(diphenylphosphane) (1.23 g, 2.13 mmol) and toluene (100 mL). The solution was sparged with N₂ for 5 min, diacetoxypalladium (581 mg, 2.59 mmol) was added, and sparging was continued. After 5 min, methyl-3-bromo-5-fluoropyridine-2-carboxylate (10.0 g, 42.7 mmol) was added, followed by portion-wise addition of (diphenylmethylene)hydrazine (8.33 g, 42.4 mmol) and cesium carbonate (27.8 g, 85.3 mmol). The reaction mixture was stirred at 70° C. under N₂ for 70 min, cooled to RT, filtered through a SiO₂ pad (120 g), and the pad was rinsed with EtOAc (800 mL). The filtrate was concentrated, and the resulting yellow solid was suspended in 4:1 heptane:EtOAc (100 mL) and stirred at RT overnight. The solids were collected by filtration, rinsed with heptane (50 mL) and dried at 40° C. under reduced pressure to provide the title compound (14.01 g, 93%) as a light yellow solid. ES-MS m/z 350 (M+H).

### Preparation 207

### Methyl 3-(2-(diphenylmethylene)-1-methylhydrazineyl)-5-fluoropicolinate

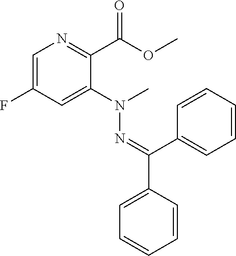 [SMILES: COC(=O)c1ncc(F)cc1N(C)N=C(c1ccccc1)c1ccccc1]

A 500 mL, 3-necked RBF equipped with nitrogen inlet, thermocouple and dropping funnel was charged with a suspension of methyl 3-(2-(diphenylmethylene)hydrazineyl)-5-fluoropicolinate (14.0 g, 40.1 mmol) in ACN (150 mL). Cesium carbonate (20.01 g, 61.41 mmol) was added portionwise, followed by dropwise addition of iodomethane (2.75 mL, 44.0 mmol) for 3 min. The reaction mixture was stirred at RT for 2 h, then at 35° C. overnight, then at 65° C. for 7 h. A second portion of iodomethane (2.50 mL, 40.0 mmol) was added and the reaction mixture was stirred at 65° C. for 10 h, cooled to RT, and stirred at RT overnight. The reaction mixture was then stirred at 65° C. for 10 additional h, cooled to RT, and stirred at RT for two days. A third portion of iodomethane (650 μL, 10.0 mmol) was added, and the reaction mixture was stirred at 65° C. for 2 h. Cesium carbonate (6.55 g, 20.1 mmol) was added, and the reaction mixture was stirred at RT for 80 minutes, filtered, and rinsed with ACN (50 mL). The filtrate was concentrated, and the residue was diluted with EtOH (100 mL) and stirred at 40° C. for 1 h, then at RT overnight. The reaction mixture was concentrated and purified by column chromatography (SiO₂, gradient 5-20% EtOAc in cyclohexane) to provide the title compound (10.4 g, 71%) as a light orange solid. ES-MS m/z 364 (M+H).

### Preparation 208

### 6-Fluoro-1-methyl-1H-pyrazolo[4,3-b]pyridin-3-ol 4-methylbenzenesulfonate

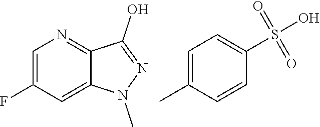

In a 3-necked, 100 mL RBF equipped with nitrogen inlet, reflux condenser and thermocouple, a suspension of methyl 3-(2-(diphenylmethylene)-1-methylhydrazineyl)-5-fluoropicolinate (3.01 g, 93% purity, 7.70 mmol) in EtOH (30 mL) was treated with p-toluenesulfonic acid monohydrate (3.02 g, 15.9 mmol). The reaction mixture was stirred at 80° C. for 12 h, then at RT for two days. The reaction mixture was filtered, and the solids were rinsed with EtOH (5 mL) and dried at 40° C. under reduced pressure. The filtrate was stirred on an ice-water bath overnight, refiltered, and rinsed with EtOH (5 mL). The solids were dried at 40° C. under reduced pressure, then combined with the first crop to provide the title compound (1.31 g, 50%) as a white solid. ES-MS m/z 168.0 (M+H).

### Preparation 209

### 6-Fuoro-3-methoxy-1-methyl-1H-pyrazolo[4,3-b]pyridine

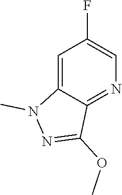

To a dried 40 mL sealed tube containing a magnetic stir bar and a mixture of 6-fluoro-1-methyl-1H-pyrazolo[4,3-b]pyridin-3-ol 4-methylbenzenesulfonate (600 mg, 1.77 mmol) and K₂CO₃ (515 mg, 3.73 mmol) in DMA (6 mL) was added Mel (120 μL, 1.92 mmol). The reaction mixture was stirred at 40° C. for 16 h, diluted with water (20 mL) and extracted with EtOAc (20 mL×3). The combined organic layers were washed with saturated aqueous NaCl (20 mL×2), dried over Na₂SO₄, filtered, and concentrated under reduced pressure. Column chromatography (SiO₂, gradient 0-40% EtOAc in hexanes) provided the title compound (184 mg, 53%) as a white solid. ES-MS m/z 181.9 (M+H).

### Preparation 210

### 5-Fluoro-2-methylnicotinonitrile

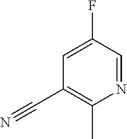

In a dried 100 mL RBF containing a magnetic stir bar, a mixture of 2-chloro-5-fluoronicotinonitrile (1.00 g, 6.39 mmol), 2,4,6-trimethyl-1,3,5,2,4,6-trioxatriborinane (3.5 M in THF, 2.20 mL, 7.70 mmol), 1,1′-bis(diphenylphosphino)ferrocene-palladium(II) dichloride (472 mg, 645 μmol) and K₂CO₃ (2.69 g, 19.5 mmol) in 1,4-dioxane (10 mL) and H₂O (2 mL) was evacuated and backfilled with N₂×3. The mixture was stirred at 120° C. for 16 h under N₂, filtered, and the filtrate was diluted with H₂O (20 mL) and extracted with EtOAc (20 mL×3). The combined organics were washed with saturated aqueous NaCl (30 mL), dried over Na₂SO₄, filtered, and concentrated under reduced pressure. Column chromatography (SiO₂, gradient 0-15% EtOAc in hexanes) provided the title compound (481 mg, 47%) as a white solid. ES-MS m/z 137.0 (M+H).

### Preparation 211

### 6-Methoxy-1-methyl-1H-pyrazolo[4,3-b]pyridine

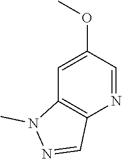

To a solution of 6-bromo-1-methyl-1H-pyrazolo[4,3-b]pyridine (700 mg, 3.30 mmol), cesium carbonate (1.61 g, 4.95 mmol), and tBuXPhos Pd G3 (348 mg, 330 μmol) in toluene (10 mL) was added methanol (1.34 mL, 33.0 mmol). The mixture was evacuated and backfilled with N₂×3, then the mixture was stirred at 80° C. for 16 h under N₂. A separate reaction was set up in the same manner using 300 mg (1.41 mmol) of 6-bromo-1-methyl-1H-pyrazolo[4,3-b]pyridine and proportional quantities of the remaining reagents. Upon completion, the reaction mixtures were combined and poured into water (20 mL). The aqueous mixture was extracted with EtOAc (30 mL×3). The combined organic layers were dried over Na₂SO₄, filtered, and concentrated under reduced pressure. Column chromatography (SiO₂, gradient 0-80% EtOAc in hexanes) provided the title compound (755 mg, 94%) as a white solid. ES-MS m/z 164.2 (M+H).

### Preparation 212

### 6-Methoxy-1-methyl-1H-pyrazolo[4,3-b]pyridine 4-oxide

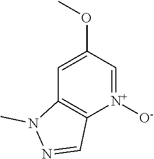

To a solution of 6-methoxy-1-methyl-1H-pyrazolo[4,3-b]pyridine (755 mg, 4.44 mmol), in EtOAc (10 mL) was added meta-chloroperoxybenzoic acid (1.15 g, 85 wt %, 5.66 mmol). The mixture was evacuated and backfilled with N₂×3, then the mixture was stirred at 25° C. for 16 h under N₂. The reaction mixture was diluted with EtOAc (20 mL), then a solution of sat. aq. NaHCO₃ (10 mL) and a solution of sat. aq. Na₂SO₃ (10 mL) were added and the mixture stirred at 25° C. for 30 min. The layers were separated, and the organic layer was dried over Na₂SO₄, filtered, and concentrated under reduced pressure to provide the title compound (543 mg, 55%) as a yellow solid. ES-MS m/z 180.1 (M+H).

The compounds in the following table were prepared in similar manner as described in preparation 212 using the appropriate pyridine or pyridine derivative. Different reaction temperatures, reaction times, meta-chloroperoxybenzoic acid loading, and purifications can be used. Such variances would be apparent to one skilled in the art.

TABLE 27

| Preparation | | | ES-MS |
|---|---|---|---|
| No. | Chemical Name | Structure | m/z |
| 213^{a} | 6-Fluoro-3- methoxy-1- methyl-1H- pyrazolo [4,3-b]pyridine 4-oxide | 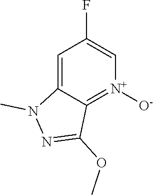 | 198.2 (M + H) |
| 214^{b} | 3-Cyano-5-fluoro-2- methylpyridine 1-oxide | 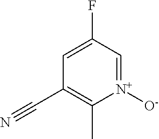 | 153.2 (M + H) |

^{a}Column chromatography (SiO₂, gradient 0-60% EtOAc in hexanes)
^{b}DCM used as solvent and for workup. Column chromatography (SiO₂, gradient 0-45% EtOAc in hexanes)

### Preparation 215

### 5-Chloro-6-methoxy-1-methyl-1H-pyrazolo[4,3-b]pyridine

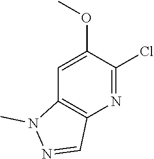

To a mixture of 6-methoxy-1-methyl-1H-pyrazolo[4,3-b]pyridine 4-oxide (1.21 g, 90 wt %, 6.08 mmol) in ACN (13 mL) was slowly added POCl₃ (2.3 mL, 25 mmol) at 0° C. The mixture was evacuated and backfilled with N₂×3, then the mixture was stirred at 25° C. for 2 h under N₂. The reaction mixture was slowly added to a solution of sat. aq. Na₂CO₃ (40 mL), then the mixture was extracted with EtOAc (70 mL×3). The combined organic layers were dried over Na₂SO₄, filtered, and concentrated under reduced pressure. Column chromatography (SiO₂, gradient 0-50% EtOAc in hexanes) provided the title compound (772 mg, 61%) as a white solid. ES-MS m/z 198.0 (M+H).

The compounds in the following table were prepared in similar manner as described in Preparation 215 using the appropriate pyridine-N-oxide. Different reaction temperatures, reaction solvents, POCl₃ loadings, and reaction workups can be used. Such variances would be apparent to one skilled in the art.

TABLE 28

| Preparation | | | ES-MS |
|---|---|---|---|
| No. | Chemical Name | Structure | m/z |
| 216 | 5-Chloro- 6-fluoro-3- methoxy-1- methyl-1H- pyrazolo [4,3-b]pyridine | 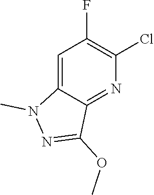 | 216.1 (M + H) |
| 217^{a} | 6-Chloro- 5-fluoro-2- methyl- nicotinonitrile | 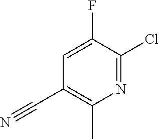 | 171.0 (M + H) |

^{a}DCE used as reaction solvent, DCM used as workup solvent.

### Preparation 218

### 3,5-Dichloro-6-methoxy-1-methyl-1H-pyrazolo[4,3-b]pyridine

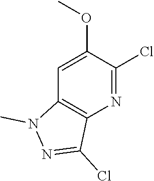

In a dry 8-mL sealed tube, 5-chloro-6-methoxy-1-methyl-1H-pyrazolo[4,3-b]pyridine (100 mg, 95 wt %, 482 μmol) was suspended in ACN (2 mL). NCS (65.2 mg, 483 μmol) was added, and the reaction mixture was stirred at 50° C. for 3 h. An additional portion of NCS (20.3 mg, 151 483 μmol) was added and the reaction mixture was stirred at 50° C. for 1 h. The reaction was cooled to RT, then the mixture was diluted with water (20 mL) and extracted with EtOAc (30 mL×2). The combined organic layers were washed with saturated aqueous NaCl (50 mL×2), dried over Na₂SO₄, filtered, and concentrated under reduced pressure. Column chromatography (SiO₂, gradient 0-2% methanol in DCM) provided the title compound (106 mg, 85%) as a white solid. ES-MS m/z 231.9 (M+H).

### Preparation 219

### 5-Bromo-6-fluoro-1-(methyl-d3)-1H-pyrazolo[4,3-b]pyridine

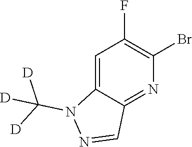 [SMILES: [2H]C([2H])([2H])n1ncc2nc(Br)c(F)cc21]

To a solution of 5-bromo-6-fluoro-1H-pyrazolo[4,3-b]pyridine (334 mg, 81 wt %, 1.25 mmol) in DMF (3 mL) at 0° C. was added NaH in mineral oil (60.1 mg, 60 wt %, 1.50 mmol). The reaction mixture was evacuated and backfilled with N₂×3, then was stirred at 0° C. for 30 min. To the resulting mixture was added iodomethane-d3 (150 μmol, 2.41 mmol) then the reaction mixture was stirred at 0° C. for 1.5 h to give a white suspension. The reaction mixture was poured into water (20 mL) and extracted with EtOAc (20 mL×3). The combined organic layers were washed with saturated aqueous NaCl (20 mL×3), dried over Na₂SO₄, filtered, and concentrated under reduced pressure. Column chromatography (SiO₂, gradient 0-60% EtOAc in hexanes) provided the title compound (207 mg, 64%) as a white solid. ES-MS m/z 232.9/234.9 (M+H).

### Preparation 220

### 6-Bromo-3-fluoro-1-methyl-1H-pyrazolo[4,3-b]pyridine

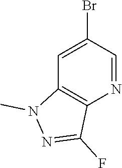

To a mixture of 6-bromo-1-methyl-1H-pyrazolo[4,3-b]pyridine (4.01 g, 18.9 mmol) in ACN (80 mL) was added 1-chloromethyl-4-fluoro-1,4-diazoniabicyclo[2.2.2]octane bis(tetrafluoroborate) (8.73 g, 24.6 mmol). The mixture was stirred at 90° C. for 16 h. The reaction mixture was poured into water (120 mL) and extracted with EtOAc (120 mL×3). The combined organic layers were dried over anhydrous Na₂SO₄, filtered, and concentrated under reduced pressure to give a residue. The residue was purified on silica gel, eluting with 0-30% EtOAc/hexanes to obtain the title compound (1.9 g, 39%) as an off-white solid. ES-MS m/z 230.0 (M+H).

The compounds in the following table were prepared in similar manner as described in Preparation 220 using the appropriate azaindazole. Different reagent loadings, reaction temperatures and times, the presence of acetic acid as an additive, and the method of purification can be used. Such variances would be apparent to one skilled in the art.

TABLE 29

| Preparation | | | ES-MS |
|---|---|---|---|
| No. | Chemical Name | Structure | m/z |
| 221^{a} | 5-Chloro-3-fluoro-6- methoxy-1-methyl-1H- pyrazolo[4,3-b]pyridine | 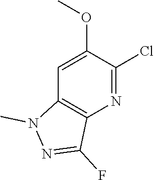 | 216.0 (M + H) |
| 222^{b} | 5-Bromo-3,6-difluoro-1- (methyl-d3)-1H- pyrazolo[4,3-b]pyridine | 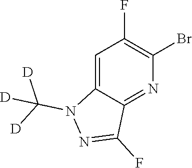 [SMILES: [2H]C([2H])([2H])n1nc(F)c2nc(Br)c(F)cc21] | 250.9/ 252.8 (M + H) |

^{a}9:1 ACN/Acetic acid used as solvent. Purified by prep-HPLC: YMC Triart C18 150*25 mm*5 μm, Mobile phase: A: H₂O (10 mM NH₄HCO₃); B: ACN, Gradient: B from 28.00% to 58.00% in 10.00 min
^{b}Purified by prep-HPLC as above with gradient B from 34.00% to 64.00%

### Preparation 223

### 3-Fluoro-1-methyl-1H-pyrazolo[4,3-b]pyridin-6-ol

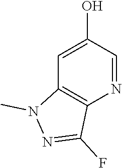

To a mixture of 6-bromo-3-fluoro-1-methyl-1H-pyrazolo[4,3-b]pyridine (1.8 g, 88% wt, 6.9 mmol) and KOH (2.37 g, 82% wt, 34.6 mmol) in 1,4-dioxane (18 mL) and water (18 mL) was added tBuXPhos Pd G3 (0.26 g, 0.32 mmol). The mixture was degassed and purged with N_{2 3} times, then the mixture was stirred at 100° C. for 3 h. The reaction mixture was poured into water (50 mL) and extracted with DCM (50 mL). The pH of aqueous phase was adjusted to 3 with 1 N HCl (aq.) and the aqueous phase was extracted by EtOAc (50 mL×3). The combined organic layers were dried over anhydrous Na₂SO₄, filtered, and concentrated in vacuo to give the title compound (951 mg, 75%) as an off-white solid. ES-MS m/z 168.0 (M+H).

### Preparation 224

### 6-(Difluoromethoxy)-3-fluoro-1-methyl-1H-pyrazolo[4,3-b]pyridine

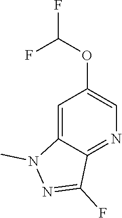

To a mixture of 3-fluoro-1-methyl-1H-pyrazolo[4,3-b]pyridin-6-ol (403 mg, 91% wt, 2.19 mmol) in DMF (10 mL) was added K₂CO₃ (1.51 g, 10.9 mmol) and sodium chloro(difluoro)acetate (2.03 g, 13.3 mmol). The mixture was stirred at 100° C. for 16 h. K₂CO₃ (919 mg, 6.65 mmol) and sodium chloro(difluoro)acetate (1.06 g, 6.95 mmol) were added to the mixture at 15° C., then the mixture was stirred at 100° C. for 2 h. Additional K₂CO₃ (912 mg, 6.60 mmol) and sodium chloro(difluoro)acetate (1.01 g, 6.62 mmol) were added to the above mixture at 15° C., then the mixture was stirred at 95° C. for 2 h. The reaction mixture was poured into water (30 mL) and extracted by EtOAc (30 mL×3). The combined organic layers were washed with saturated aqueous NaCl (20 mL), dried over anhydrous Na₂SO₄, filtered, and concentrated in vacuo to give a residue. The residue was purified on silica gel, eluting with 0-60% EtOAc/hexanes to obtain the title compound (131 mg, 25%) as a purple solid. ES-MS m/z 218.0 (M+H).

### Preparation 225

### 6-(Difluoromethoxy)-3-fluoro-1-methyl-1H-pyrazolo[4,3-b]pyridine 4-oxide

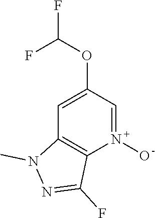

To a mixture of 6-(difluoromethoxy)-3-fluoro-1-methyl-1H-pyrazolo[4,3-b]pyridine (130 mg, 90% wt, 539 μmol) in EtOAc (3 mL) was added meta-chloroperoxybenzoic acid (133 mg, 85% wt, 655 μmol) at 15° C. The mixture was degassed and refilled with N₂ 3 times, then the mixture was stirred at 15° C. for 16 h under N₂ atmosphere. Additional meta-chloroperoxybenzoic acid (216 mg, 85% wt, 1.06 mmol) was added to the mixture and the mixture was stirred at 30° C. for 16 h under N₂ atmosphere. Additional meta-chloroperoxybenzoic acid (105 mg, 85% wt, 517 μmol) was added to the mixture and the mixture was stirred at 30° C. for 16 h under N₂ atmosphere. The mixture was directly purified by flash silica gel chromatography without any workup, eluting with 0-80% EtOAc/hexanes to obtain the title compound (92 mg, 66%) as a pale pink solid. ES-MS m/z 234.0 (M+H).

### Preparation 226

### 5-chloro-6-(difluoromethoxy)-3-fluoro-1-methyl-1H-pyrazolo[4,3-b]pyridine

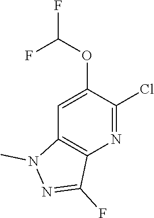 [SMILES: Cn1nc(F)c2nc(Cl)c(OC(F)F)cc21]

To a mixture of 6-(difluoromethoxy)-3-fluoro-1-methyl-1H-pyrazolo[4,3-b]pyridine 4-oxide (92 mg, 90% wt, 0.36 mmol) in ACN (2 mL) was added phosphorus oxychloride (266 mg, 160 μL, 1.74 mmol) at 0° C. The mixture was degassed and refilled with N₂ 3 times, then the mixture was stirred at RT for 3 days. The reaction mixture was slowly added to a sat. NaHCO₃ aqueous solution (10 mL), then extracted with EtOAc (10 mL×3). The combined organic layers were dried over Na₂SO₄ and concentrated in vacuo. The resulting residue was purified by SFC [column: Princeton SFC 4-ETH (250 mm×30 mm, 10 μm); mobile phase: 20% IPA in CO₂+0.1% NH₄OH); flowrate (mL/min): 60] to provide the title compound (80 mg, 90%) as a pale yellow solid. ES-MS m/z 252.0 (M+H).

### Preparation 227

### 1-Methyl-5-(tributylstannyl)-1H-pyrazolo[4,3-b]pyridine-6-carbonitrile

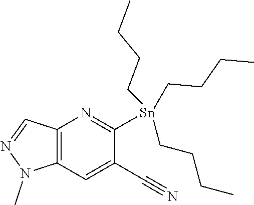 [SMILES: CCC[CH2][Sn]([CH2]CCC)([CH2]CCC)[c]1nc2cnn(C)c2cc1C#N]

A mixture of 5-chloro-1-methyl-1H-pyrazolo[4,3-b]pyridine-6-carbonitrile (196.5 mg, 1.02 mmol) and bis(di-tert-butyl (4-dimethylaminophenyl)phosphine)dichloropalladium(II) (71.8 mg, 101 μmol) was placed under an atmosphere of argon and then 1,4-dioxane (10.0 mL) was added followed by 1,1,1,2,2,2-hexabutyldistannane (770 μL, 1.53 mmol). Stirring was initiated, and the reaction was heated to 105° C. for 18.1 h. The reaction was then cooled to RT and quenched by the addition of 6 mL of 1 M aq. KF and diluted with saturated aqueous NaCl and EtOAc. The organic layer was removed, and the aqueous layer was extracted with EtOAc (3×). The combined organic layers were then washed with saturated aqueous NaCl, dried over Na₂SO₄, filtered, concentrated, and purified by silica gel chromatography using a gradient of 0-50% EtOAc in heptane to give the title compound (132.2 mg, 26%) as a yellow oil. ES-MS m/z 449.2 (M+H).

The compound in the following table was prepared in similar manner as described in Preparation 227 using the appropriate 5-haloazaindazole but varying the 5-halogen from chloride to bromide. Different reaction times, concentration, workup, and purification can be used. Such variances would be apparent to one skilled in the art.

TABLE 30

| Preparation | | | ES-MS |
|---|---|---|---|
| No. | Chemical Name | Structure | m/z |
| 228^{a} | 6-Fluoro-1-methyl-5- (tributylstannyl)-1H- pyrazolo[4,3-b]pyridine | 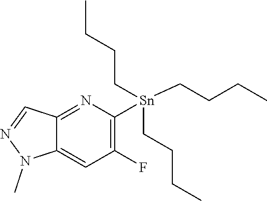 [SMILES: CCC[CH2][Sn]([CH2]CCC)([CH2]CCC)[c]1nc2cnn(C)c2cc1F] | (M + H) 441.9 |

^{a}0.3M reaction, 16 h reaction time, the crude reaction mixture was concentrated under reduced pressure and directly purified by silica gel chromatography using a gradient of 0-20% EtOAc in hexanes.

### Preparation 229

### 6-Chloro-5-methoxy-2-methylpyridazin-3 (2H)-one

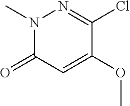

To a mixture of 5,6-dichloro-2-methylpyridazin-3 (2H)-one (9.98 g, 55.8 mmol) in anhydrous MeOH (110 mL) under an atmosphere of N₂ was added a solution of sodium methanolate (12.7 mL, 25 wt % in MeOH, 55.5 mmol). The reaction vessel was fitted with a reflux condenser and then stirred at 50° C. for 90 min, after which it was cooled to RT and concentrated under reduced pressure. The resulting residue was washed with water, and the solids were collected by filtration. The filter cake was rinsed with hexane and then collected and lyophilized to give the title compound (8.65 g, 89%) as a white solid. ES-MS m/z 174.8 (M+H).

### Preparation 230

### 5,6-Dichloro-2-methoxynicotinonitrile

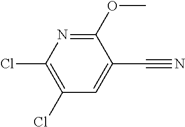

To a solution of 6-chloro-2-methoxypyridine-3-carbonitrile (498 mg, 2.95 mmol) in acetic acid (10 mL) at 25° C. was added NCS (1.6 g, 12 mmol). The mixture was then heated to 120° C. and stirred for 16 h, after which it was cooled to RT and quenched by the addition of sat. aq. Na₂CO₃ (100 mL) and extracted with EtOAc (2×50 mL). The combined organic layers were washed with saturated aqueous NaCl (2× 30 mL), dried over Na₂SO₄, filtered, concentrated under reduced pressure, and purified by silica gel chromatography using a gradient of 0-13% EtOAc in hexanes followed by prep-HPLC (F-Welch Xtimate C18 40*200 mm 7 μm; mobile phase: [A: H₂O (0.225% HCO₂H); B: MeOH]; 28-68% B, 20.00 min; flow rate: 60.00 ml/min) and lyophilized to give the title compound (162 mg, 27%) as a white solid. NMR: ¹H NMR (DMSO-d6) δ 8.71 (s, 1H), 4.00 (s, 3H).

### Preparation 231

### 2-Chloro-6-methylpyridine-3,5-dicarbonitrile

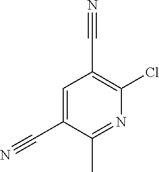

To a solution of 5-bromo-2-chloro-6-methylnicotinonitrile (200 mg, 864 μmol), trimethylsilyl cyanide (0.23 mL, 1.73 mmol) and zinc fluoride (53.6 mg, 518 μmol) in DMA (10 mL) was added XantPhos-Pd-G2 (154 mg, 173 μmol). The mixture was degassed and purged 3× with N₂, and then the mixture was stirred at 80° C. for 16 h under an atmosphere of N₂. The reaction was then diluted with H₂O (30 mL) and extracted with EtOAc (3×25 mL). The combined organic layers were washed with saturated aqueous NaCl (2×30 mL), dried over Na₂SO₄, filtered, concentrated under reduced pressure, and purified by silica gel chromatography using a gradient of 0-20% EtOAc in hexanes to give the title compound (112 mg, 69%) as a yellow solid. NMR: ¹H NMR (DMSO-d6) δ 9.18 (s, 1H), 2.61 (s, 3H).

### Preparation 232

### 1-(Bromodifluoromethyl)-5-chloro-4-methoxypyridin-2(1H)-one

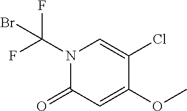

To a solution of 5-chloro-4-methoxypyridin-2(1H)-one (250 mg, 1.57 mmol) in DMF (4 mL) in a sealed tube cooled to 0° C. was added sodium hydride (125 mg of a 60 wt % dispersion in mineral oil, 3.13 mmol). The reaction mixture was stirred at 0° C. for 0.5 h. Then dibromodifluoromethane (1.41 mL, 15.7 mmol) was added and the reaction was stirred at RT for 16 h. The reaction was then diluted with EtOAc (30 mL) and washed with water (3×30 mL). The organic layer was dried over Na₂SO₄, filtered, concentrated under reduced pressure, and purified by silica gel chromatography using a gradient of 0˜25% EtOAc in hexane to give the title compound (47 mg, 10%) as a yellow oil. ES-MS m/z 287.9, 289.9, 291.9 (M+H).

### Preparation 233

### 5-Chloro-4-methoxy-1-(trifluoromethyl)pyridin-2(1H)-one

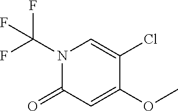

To a solution of 1-(bromodifluoromethyl)-5-chloro-4-methoxypyridin-2(1H)-one (47 mg, 96% wt, 0.16 mmol) in DCM (1.5 mL) was added silver (I) tetrafluoroborate (33 mg, 0.17 mmol). The mixture was stirred at RT for 1 h. Then, the mixture was diluted with DCM (5 mL) and filtered. The filtrate was concentrated under reduced pressure. The residue was purified by prep-TLC (1:1 hexanes/EtOAc, rf=0.5) to give the title compound (20 mg, 52%) as a white solid. ES/MS m/z 228.0/230.0 (M+H)

### Preparation 234

### Methyl 2-bromo-6-methylimidazo[2,1-b]thiazole-3-carboxylate

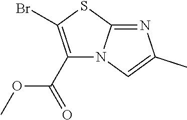

A mixture of 2-amino-5-bromo-thiazole-4-carboxylic acid methyl ester (1.00 g, 4.23 mmol), ethanol (20 mL), and bromoacetone (588 mg, 360 μL, 4.29 mmol) was heated to 100° C. overnight. The reaction was cooled to RT and concentrated under reduced pressure. The residue was redissolved in EtOAc and poured into sat. aq. NaHCO₃. The layers were separated, and the aqueous layer was extracted with EtOAc (×2). The combined organic layer was washed with saturated aqueous NaCl, dried over anhydrous Na₂SO₄, filtered and concentrated under reduced pressure. The residue was purified by column chromatography (SiO₂, gradient 0-100% EtOAc/heptane) to give the title compound (81.8 mg, 6.5%) as a yellow solid. ES/MS m/z 273.2/275.0 (M+H)

### Preparation 235

### 2-Bromo-6-methylimidazo[2,1-b]thiazole-3-carboxamide

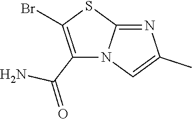

A mixture of methyl 2-bromo-6-methylimidazo[2,1-b]thiazole-3-carboxylate (81.8 mg, 297 μmol) and ammonia in methanol (7 M, 1 mL) was heated to 50° C. for 15 h. After 15 h, the heat was turned off and the reaction was stirred at RT for about 48 h. A mixture of DCM and MeOH were added, and the reaction was concentrated under reduced pressure to give the title compound (64.5 mg, 75%). ES/MS m/z 260.0/262.0 (M+H)

### Preparation 236

### 2-Bromo-6-methylimidazo[2,1-b]thiazole-3-carbonitrile

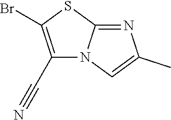

To a mixture of 2-bromo-6-methylimidazo[2,1-b]thiazole-3-carboxamide (64.5 mg, 248 μmol) in DCM (3 mL) was added TEA (65 mg, 90 μL, 0.65 mmol). After cooling the reaction to 0° C., trifluoroacetic anhydride (75 mg, 50 μL, 0.36 mmol) was added. After 5 min, the ice bath was removed, and the reaction was stirred at RT for 30 min. The reaction was quenched by the addition of MeOH and concentrated under reduced pressure. The crude mixture was redissolved in EtOAc and washed with sat. aq. NH₄Cl followed by sat. aq. NaHCO₃. The organic layer was dried over anhydrous Na₂SO₄, filtered and concentrated under reduced pressure. The solid residue was triturated with MeOH and filtered. The MeOH filtrate was removed and then the solid was dissolved in EtOAc. The EtOAc filtrate was concentrated under reduced pressure to give the title compound (33.7 mg, 53%) as a white solid. ES/MS m/z 242.0/244.0 (M+H)

### Preparation 237

### 1-Cyclopropyl-4-iodopyridin-2(1H)-one

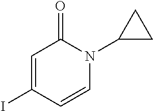 [SMILES: O=c1cc(I)ccn1C1CC1]

A mixture of 4-iodopyridin-2(1H)-one (8.0 g, 36 mmol), copper diacetate (6.98 g, 1.6 mL, 38.4 mmol), 2,2′-bipyridine (6.0 g, 5.1 mL, 38 mmol), cyclopropylboronic acid (7.0 g, 2.81 mmol) and sodium carbonate (8.7 g, 5.9 mL, 82 mmol) in 1,2-DCE (240 mL) was heated to 70° C. under air atmosphere for 15 h. The reaction mixture was cooled to RT, quenched with sat. aq. NH₄Cl and extracted with DCM. The organic layer was dried over anhydrous Na₂SO₄, filtered, and concentrated under reduced pressure. The residue was purified by column chromatography (SiO₂, gradient 0-100% EtOAc/CHX followed by gradient 0-50% MeOH/DCM) to give the title compound (1.05 g, 9.4%) as a yellow oil. ES/MS m/z 261.6 (M+H)

### Preparation 238

### 5-Bromo-1-(methyl-d3)pyridin-2(1H)-one

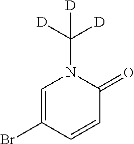 [SMILES: [2H]C([2H])([2H])n1cc(Br)ccc1=O]

To a solution of 5-bromo-1H-pyridin-2-one (2.0 g, 11 mmol) in DMF (20 mL) at 0° C. was added 60% NaH in mineral oil (673 mg, 60% wt, 16.8 mmol). The reaction mixture was degassed and refilled with N₂ for 3 times. After stirring at 0° C. for 0.5 h, iodomethane-d3 (4.4 g, 1.9 mL, 2.7 eq, 31 mmol) was added in one portion, then the reaction mixture was stirred at 0° C. for 1.5 h to give a white suspension. The reaction mixture was poured into water and extracted with EtOAc. The combined organic layers were washed with saturated aqueous NaCl, dried over anhydrous Na₂SO₄, filtered and concentrated under reduced pressure to give a crude product. The crude product was purified by flash silica gel chromatography (eluent of 0˜4% methanol/dichloromethane gradient) to give the title compound (1.68 g, 69%) as yellow oil. ES/MS m/z: (⁷⁹Br/⁸¹Br) 190.9, 192.9 (M+H).

### Preparation 239

### 1-(5-Bromopyrimidin-2-yl)-N,N-dimethylmethanamine

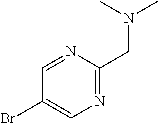

To a mixture of 5-bromo-2-(bromomethyl)pyrimidine (500 mg, 1.98 mmol) in THF (5 mL) was added 40% dimethylamine in water (1.12 g, 2.00 mmol). The reaction mixture was stirred at 24° C. for 12 h, then quenched by adding 2M NaOH (aq.) and extracted with EtOAc (20 mL×2). The combined organic layers were washed with saturated aqueous NaCl (20 mL×2), dried over Na₂SO₄, filtered and concentrated under reduced pressure. The title compound (334 mg, 74%) was obtained as a yellow solid. ES/MS m/z: (⁷⁹Br/⁸¹Br) 215.8, 217.8 (M+H).

### Preparation 240

### 4-Bromo-1-(difluoromethyl)pyridin-2(1H)-one

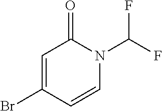 [SMILES: O=c1cc(Br)ccn1C(F)F]

To a mixture of 4-bromopyridin-2(1H)-one (2.10 g, 12.1 mmol) in DMF (50 mL) was added sodium chloro(difluoro)acetate (3.8 g, 25 mmol) and Cs₂CO₃ (7.6 g, 23 mmol), The reaction mixture was stirred at 100° C. for 16 h. After cooling down to RT, the reaction mixture was diluted with water (300 mL) and extracted with EtOAc (250 mL×2). The combined organic layers were washed with saturated aqueous NaCl (200 mL×3), dried over anhydrous Na₂SO₄, filtered and concentrated under reduced pressure. The residue was purified by flash silica gel chromatography (eluent of 0˜12% EtOAc/hexanes gradient) to give the title compound (385 mg, 14%) as a colorless oil. ES/MS m/z: (⁷⁹Br/⁸¹Br) 223.8, 225.9 (M+H).

### Preparation 241

### 3-Benzoyl-1-cyclopropylpyrimidine-2,4(1H,3H)-dione

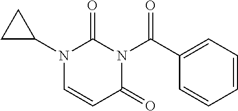 [SMILES: O=C(c1ccccc1)n1c(=O)ccn(C2CC2)c1=O]

To a solution of 3-benzoylpyrimidine-2,4(1H,3H)-dione (24.1 g, 90% wt, 100 mmol) and cyclopropylboronic acid (21.1 g, 246 mmol) in 1,2-dichloroethane (450 mL) were added copper (II) acetate (17.9 g, 98.6 mmol) and 2,2′-bipyridine (16.1 g, 103 mmol) and sodium carbonate (22 g, 0.21 mol) at 20° C. The reaction mixture was degassed and refilled with O₂ for 3 times at 20° C., Then the reaction mixture was stirred at 75° C. for 16 h under O₂ (15 psi). The mixture was cooled to RT, EtOAc was added (500 mL), then filtered through a pad of diatomaceous earth and washed with EtOAc (400 mL). The filtrate was concentrated under reduced pressure to give the residue as a green oil. The residue was triturated with EtOAc/MeOH/Hexanes (v:v:v=1:1:1, 150 mL) at 20° C. for 0.5 h. The mixture was filtered to give a cake, then the filter cake was dried under reduced pressure to give the title compound (4.5 g, 9.1%) as a light-green solid. ES/MS m/z: 257.0 (M+H).

### Preparation 242

### 4-Chloro-1-cyclopropylpyrimidin-2 (1H)-one

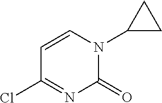 [SMILES: O=c1nc(Cl)ccn1C1CC1]

A suspension of 3-benzoyl-1-cyclopropylpyrimidine-2,4(1H,3H)-dione (1.49 g, 90% wt, 5.23 mmol) in phosphoryl trichloride (20 g, 12 mL, 0.13 mol) was degassed and purged with nitrogen three times at 20° C. The reaction mixture was stirred at 70° C. for 16 h under the nitrogen atmosphere. After completion, the mixture was cooled to RT and concentrated under reduced pressure, yielding a dark oil. The crude residue was then diluted with DCM (30 mL) and slowly added to ice-cold saturated aqueous sodium bicarbonate (40 mL). The organic phase was separated, dried over anhydrous sodium sulfate, and filtered. The filtrate was concentrated under reduced pressure, affording a brown solid. The resulting solid was triturated with a DCM/hexane mixture (1:10 v/v, 20 mL) at 20° C. for 30 min. The mixture was then filtered to obtain a solid cake, which was subsequently dried under reduced pressure to yield the title compound as a yellow solid (465 mg, 42%). ES/MS m/z: ⁷⁹Br/⁸¹Br 170.9/172.9 (M+H).

### Preparation 243

### 4-Bromo-1-cyclopropylpyridin-2(1H)-one

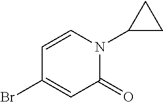 [SMILES: O=c1cc(Br)ccn1C1CC1]

To a solution of 4-bromopyridin-2(1H)-one (10.0 g, 56.5 mmol) in DCE (150 mL) was added cyclopropylboronic acid (9.81 mg, 113 mmol), 4 A molecular sieves (5 g), and Na₂CO₃ (12.3 g, 114 mmol). The reaction mixture was stirred at 25° C. for 5 min. A suspension of copper diacetate (10.4 g, 56.6 mmol) and 2,2′-bipyridine (8.93 g, 56.6 mmol) in DCE (100 mL) was heated to 50° C. and the hot suspension was added to the above reaction mixture. The reaction mixture was evacuated and backfilled with O₂×3 then was stirred at 70° C. for 16 h under O₂ (15 psi). The reaction mixture was cooled to RT, then the mixture was filtered, and the filter cake was washed with EtOAc (50 mL×3). The filtrate was concentrated under reduced pressure. Column chromatography (SiO₂, gradient 0-35% EtOAc in hexanes) provided the title compound (9.17 g, 64%) as a white solid. ES-MS m/z 213.9/215.9 (M+H).

### Preparation 244

### (1-Cyclopropyl-2-oxo-1,2-dihydropyridin-4-yl)boronic acid

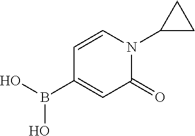 [SMILES: O=c1cc(B(O)O)ccn1C1CC1]

To a mixture of 4-bromo-1-cyclopropylpyridin-2(1H)-one (1.98 g, 85% purity, 7.86 mmol) and bis(pinacolato)diborane (2.64 g, 10.3 mmol) in 1,4-dioxane (30 mL) was added potassium acetate (2.36 g, 23.8 mmol) and PdCl₂(dppf) (602.3 mg, 814.9 μmol). The mixture was degassed and purged with N₂ (3×). The resulting mixture was gradually heated up to 100° C. and stirred at 100° C. for 16 h under N₂. The reaction mixture was filtered, and the filter cake was washed with EtOAc (3×20 mL). The combined filtrate was concentrated under reduced pressure. The residue was purified by reversed-phase preparative HPLC (Welch Xtimate® C18, 40×200 mm, 7 μm, gradient 0-20% ACN in 0.225% aqueous FA) to provide the title compound (515 mg, 35%) as a pink solid. ES-MS m/z 180.0 (M+H).

### Preparation 245

### 3-Acetyl-1-methylpyrrolidin-2-one (racemic mixture)

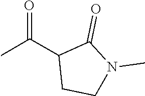

A solution of 1-methylpyrrolidin-2-one (50.0 g, 504 mmol) in THF (400 mL) was degassed and purged with N₂ (3×). The solution was cooled to −78° C. and lithium diisopropylamide (2.0 M in THF, 280 mL, 560 mmol) was added dropwise over 60 min. The reaction mixture was stirred at −78° C. for 2 h, then methyl acetate (80 mL, 1000 mmol) was added dropwise at −78° C. The resulting mixture was allowed to warm to 20° C. and stirred for 16 h. The reaction mixture was treated with 1N aq HCl (1000 mL) and extracted with 10:1 DCM: MeOH (3×500 mL). The organic layers were combined, dried over anhydrous Na₂SO₄, filtered, and concentrated under reduced pressure. The residue was purified on silica, eluting with 0-4% MeOH in DCM, to provide the title compound (44.3 g, 59%) as a yellow oil. ES-MS m/z 142.1 (M+H).

### Preparation 246

### 4,4,4-Trifluoro-1-(1-methyl-2-oxopyrrolidin-3-yl)butane-1,3-dione (racemic mixture)

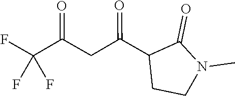 [SMILES: CN1CCC(C(=O)CC(=O)C(F)(F)F)C1=O]

A solution of 3-acetyl-1-methylpyrrolidin-2-one (racemic mixture) (20.3 g, 95% purity, 137 mmol) in THF (300 mL) was degassed and purged with N₂ (3×). The solution was cooled to 0° C. and lithium diisopropylamide (2.0 M in THF, 200 mL, 400 mmol) was added dropwise. The reaction mixture was stirred at 0° C. for 1 hour, then the mixture was cooled to −78° C., and ethyl 2,2,2-trifluoroacetate (21 mL, 180 mmol) was added dropwise. The resulting mixture was stirred at −78° C. for 1 hour. The mixture was diluted with MeOH (300 mL) and concentrated under reduced pressure. The residue was purified on silica, eluting with 0-4% MeOH in DCM, to obtain the title compound (27.1 g, 79%) as a yellow oil. ES-MS m/z 238.2 (M+H).

### Preparation 247

### 3-(1-(2-Amino-2-methylpropyl)-3-(trifluoromethyl)-1H-pyrazol-5-yl)-1-methylpyrrolidin-2-one (Racemic Mixture)

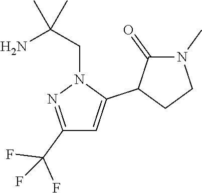 [SMILES: CN1CCC(c2cc(C(F)(F)F)nn2CC(C)(C)N)C1=O]

A mixture of 4,4,4-trifluoro-1-(1-methyl-2-oxopyrrolidin-3-yl)butane-1,3-dione (racemic mixture) (900 mg, 95% purity, 3.60 mmol), 1-hydrazineyl-2-methylpropan-2-amine trihydrochloride (965 mg, 90% purity, 4.09 mmol) and TEA (2.1 mL, 15 mmol) in EtOH (15 mL) was stirred at 80° C. for a total of 32 h. The reaction mixture was directly purified by reversed-phase preparative HPLC (Welch Xtimate® C18, 40×200 mm, 7 μm, gradient 10-50% ACN in 10 mM aq NH₄HCO₃ with 0.05% NH₄OH) to provide the title compound (160 mg, 15%) as a yellow oil. ES-MS m/z 305.1 (M+H).

### Preparation 248

### tert-Butyl (2-methyl-1-(3-(trifluoromethyl)-1H-pyrazol-1-yl)propan-2-yl)carbamate

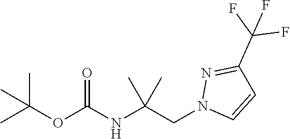 [SMILES: CC(C)(Cn1ccc(C(F)(F)F)n1)NC(=O)OC(C)(C)C]

To a solution of tert-butyl 4,4-dimethyl-1,2,3-oxathiazolidine-3-carboxylate 2,2-dioxide (3.80 kg, 15.1 mol) in DMF (26.6 L) was added Cs₂CO₃ (8.21 kg, 25.2 mol) and 3-(trifluoromethyl)-1H-pyrazole (1.71 kg, 12.6 mol). The mixture was stirred at 75° C. for 4 h. The residue was diluted with H₂O (38 L) and extracted with EtOAc (3×19 L). The combined organic layers were washed with saturated aqueous NaCl (3×19 L), dried over Na₂SO₄, filtered and concentrated under reduced pressure to give a residue. The residue was treated with n-heptane (11.4 L) then concentrated under reduced pressure (2×). The residue was resuspended in n-heptane (11.4 L), filtered, and the filter cake was dried to obtain the title compound (2.50 kg, 64.5%) as white solid. ES-MS m/z 208.1 (M+H-Boc), 252.0 (M+H-tBu).

The compounds in the following table were prepared in similar manner as described in Preparation 248 using the appropriate pyrazole. Different reaction temperatures and reaction times can be used. Such variances would be apparent to one skilled in the art.

TABLE 31

| Preparation | | | ES-MS |
|---|---|---|---|
| No. | Chemical Name | Structure | m/z |
| 249^{ab} | tert-Butyl (1-(5- methoxy-3- (trifluoromethyl)-1H- pyrazol-1-yl)-2- methylpropan-2- yl)carbamate | 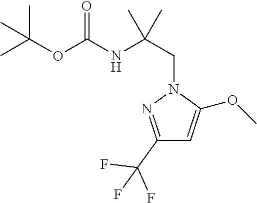 [SMILES: COc1cc(C(F)(F)F)nn1CC(C)(C)NC(=O)OC(C)(C)C] | 338.3 (M + H) |
| 250^{ab} | Ethyl 1-(2-((tert- butoxycarbonyl)amino)- 2-methylpropyl)-3- (trifluoromethyl)-1H- pyrazole-5-carboxylate | 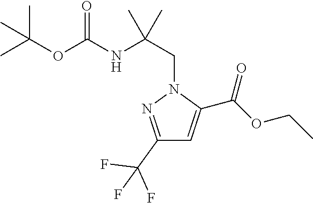 [SMILES: CCOC(=O)c1cc(C(F)(F)F)nn1CC(C)(C)NC(=O)OC(C)(C)C] | 280.1 (M + H − Boc) |

^{a}The crude product was purified by prep-HPLC (column: YMC Triart C18 250*30 mm*5 μm; mobile phase: [A: H₂O(10 mM NH₄HCO₃); B: ACN] and lyophilized
^{b}Dimethylacetamide solvent was used in place of the DMF solvent.

### Preparation 251

### tert-Butyl (1-(5-(1,3,6,2-dioxazaborocan-2-yl)-3-(trifluoromethyl)-1H-pyrazol-1-yl)-2-methylpropan-2-yl)carbamate

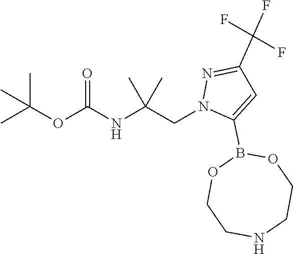 [SMILES: CC(C)(Cn1nc(C(F)(F)F)cc1B1OCCNCCO1)NC(=O)OC(C)(C)C]

Under nitrogen atmosphere using deoxygenated solvent, a solution of bis(pinacolato)diboron (2.18 kg, 8.59 mol) in 2-methyltetrahydrofuran (11.0 L) was treated with 4,4′-di-tert-butyl-2,2′-dipyridyl (37.5 g, 0.14 mol) and (1,5-cyclooctadiene)(methoxy)iridium (I) dimer (CAS 12148-71-9, 46.3 g. 0.07 mol) at 25° C. The mixture was stirred at 25° C. for 0.5 h, then a solution of tert-butyl (2-methyl-1-(3-(trifluoromethyl)-1H-pyrazol-1-yl)propan-2-yl)carbamate (2.20 kg, 7.16 mol) in 2-methyltetrahydrofuran (4.40 L) was added. The resulting mixture was stirred at 70° C. for 16 h, then cooled to 0-5° C. and treated with a solution of diethanolamine (1.50 kg, 14.3 mol) in IPA (2.20 L) dropwise over 2 h at 0-5° C. The mixture was stirred at 25° C. for 16 h, then it was filtered. The filter cake was washed with MTBE (4.40 L), then the remaining solids were stirred with MTBE: IPA (1:1, 11.0 L) at 25° C. for 16 h. The mixture was filtered, and the filter cake was vacuum dried to provide the title compound (2.10 kg, 67%) as white solid. ES-MS m/z 252.0 (M+H-Boc-diethanolamine, DEA), 296.0 (M+H-tBu-DEA).

### Preparation 252

### tert-Butyl (2-methyl-1-(5-(4,4,5,5-tetramethyl-1,3,2-dioxaborolan-2-yl)-3-(trifluoromethyl)-1H-pyrazol-1-yl)propan-2-yl)carbamate

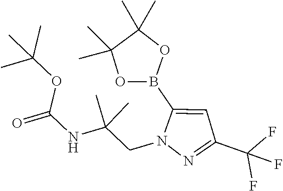 [SMILES: CC(C)(Cn1nc(C(F)(F)F)cc1B1OC(C)(C)C(C)(C)O1)NC(=O)OC(C)(C)C]

In a N₂ filled glovebox, bis(pinacolato)diboron (5.5 g, 22 mmol), (1,5-cyclooctadiene)(methoxy)iridium (I) dimer (0.36 g, 0.55 mmol) and 4,4′-di-tert-butyl-2,2′-dipyridyl (0.29 g, 1.1 mmol) were combined in a dried reaction vessel. 2-Methyltetrahydrofuran (40 mL) was added, and the reaction mixture was stirred at 25° C. for 0.5 h. A solution of tert-butyl (2-methyl-1-(3-(trifluoromethyl)-1H-pyrazol-1-yl)propan-2-yl)carbamate (5.7 g, 98% purity, 18 mmol) in 2-methyltetrahydrofuran (60 mL) was added, and the resulting reaction mixture was stirred at 70° C. for 16 h. The reaction mixture was filtered, and the filtrate was concentrated under reduced pressure. The residue was purified on silica, eluting with 0-1% MeOH in DCM, to obtain the title compound (7.3 g, 88%) as a white solid. ES-MS m/z 434.0 (M+H).

### Preparation 253

### tert-Butyl (2-methyl-1-(4-(trifluoromethyl)-1H-imidazol-2-yl)propan-2-yl)carbamate

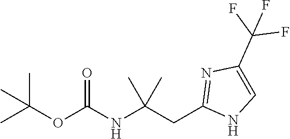 [SMILES: CC(C)(Cc1nc(C(F)(F)F)c[nH]1)NC(=O)OC(C)(C)C]

To a solution of tert-butyl (2-methyl-4-oxobutan-2-yl)carbamate (12.02 g, 59.72 mmol) in EtOH (180 mL) was added NH₄OH (65.0 mL, 28 wt %, 470 mmol). The reaction mixture was cooled to 0° C. and 1,1,1-trifluoro-3,3-dibromoacetone (120 mL, 881 mmol) was added. The reaction mixture was then stirred at 25° C. for 16 h, poured into water (300 mL), and extracted with EtOAc (300 mL×2). The combined organics were dried over Na₂SO₄, filtered, and concentrated under reduced pressure. Two rounds of column chromatography (first round: SiO₂, gradient 0-4% MeOH in CH₂Cl₂; second round: SiO₂, gradient 0-2% MeOH in CH₂Cl₂) provided the title compound (9.4 g, 50%) as a white solid.

### Preparation 254

### tert-Butyl (1-(5-(1-cyclopropyl-2-oxo-1,2-dihydropyridin-4-yl)-3-(trifluoromethyl)-1H-pyrazol-1-yl)-2-methylpropan-2-yl)carbamate

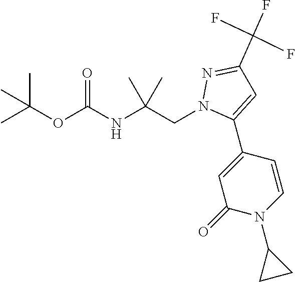 [SMILES: CC(C)(Cn1nc(C(F)(F)F)cc1-c1ccn(C2CC2)c(=O)c1)NC(=O)OC(C)(C)C]

A 2 L RBF with a magnetic stirring bar was charged with 4-bromo-1-cyclopropylpyridin-2(1H)-one (15.5 g, 72.4 mmol), Na₂CO₃ (22.3 g, 210 mmol), [1,1′-bis(diphenylphosphino)ferrocene]dichloropalladium(II)·CH₂Cl₂ (5.91 g, 7.24 mmol), 1,4-dioxane (280 mL), IPA (64 mL) and water (210 mL). The resultant mixture was purged with nitrogen for 3 min, then tert-butyl (1-(5-(1,3,6,2-dioxazaborocan-2-yl)-3-(trifluoromethyl)-1H-pyrazol-1-yl)-2-methylpropan-2-yl)carbamate (32.4 g, 77.1 mmol) was added. The reaction mixture was purged with nitrogen for 10 min, sealed with a septum, equipped with a nitrogen balloon, and heated at 65° C. for 3 h. The reaction mixture was filtered through a pad of diatomaceous earth, washing with EtOAc (2×150 mL). Water (300 mL) was added to the filtrate, and the aqueous phase was separated and extracted with EtOAc (2×150 mL). Combined organics were washed with saturated aqueous NaCl (300 mL), dried over MgSO₄, filtered, and concentrated under reduced pressure. The residue was suspended in 30% EtOAc in hexane (50 mL) and sonicated. A light brown solid precipitated slowly. The solid was filtered and the precipitate was resuspended in MTBE (568 mL) and stirred at 50° C. for 1 h. The suspension was filtered and the filtrate evaporated to give the title compound (24.1 g, 74%) as a pale yellow solid. ES-MS m/z 441.2 (M+H).

The compounds in the following table were prepared in similar manner as described in Preparation 254 using the appropriate (hetero) aryl halide. Different reaction temperatures and reaction times can be used. Such variances would be apparent to one skilled in the art. The catalyst can be substituted with (2-dicyclohexylphosphino-2′,4′,6′-triisopropyl-1,1′-biphenyl)[2-(2′-amino-1,1′-biphenyl)]palladium(II) methanesulfonate, or [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate. The base may be substituted with K₃PO₄. Purification may be accomplished by column chromatography on silica gel.

TABLE 32

| Preparation | | | ES-MS |
|---|---|---|---|
| No. | Chemical Name | Structure | m/z |
| 255^{a} | tert-Butyl (2-methyl-1- (5-(1-(methyl-d3)-6- oxo-1,6-dihydropyridin- 3-yl)-3- (trifluoromethyl)-1H- pyrazol-1-yl)propan-2- yl)carbamate | 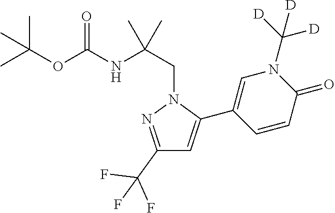 [SMILES: [2H]C([2H])([2H])n1cc(-c2cc(C(F)(F)F)nn2CC(C)(C)NC(=O)OC(C)(C)C)ccc1=O] | 418.3 (M + H) |
| 256^{b} | tert-Butyl (1-(5-(2- ((dimethylamino)methyl) pyrimidin-5-yl)-3- (trifluoromethyl)-1H- pyrazol-1-yl)-2- methylpropan-2- yl)carbamate | 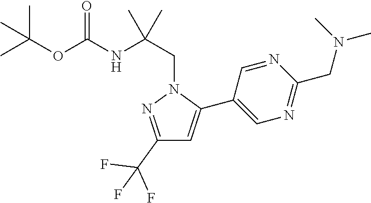 [SMILES: CN(C)Cc1ncc(-c2cc(C(F)(F)F)nn2CC(C)(C)NC(=O)OC(C)(C)C)cn1] | 443.1 (M + H) |
| 257^{c} | tert-Butyl (1-(5-(1- cyclobutyl-2-oxo-1,2- dihydropyridin-4-yl)-3- (trifluoromethyl)-1H- pyrazol-1-yl)-2- methylpropan-2- yl)carbamate | 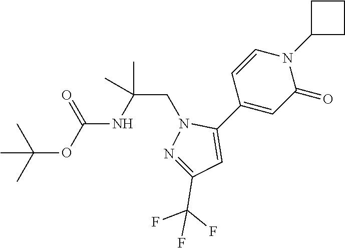 [SMILES: CC(C)(Cn1nc(C(F)(F)F)cc1-c1ccn(C2CCC2)c(=O)c1)NC(=O)OC(C)(C)C] | 455.2 (M + H) |
| 258^{d} | tert-Butyl (1-(5-(1- (difluoromethyl)-2-oxo- 1,2-dihydropyridin-4- yl)-3-(trifluoromethyl)- 1H-pyrazol-1-yl)-2- methylpropan-2- yl)carbamate | 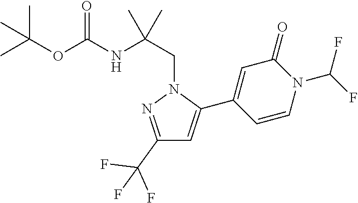 [SMILES: CC(C)(Cn1nc(C(F)(F)F)cc1-c1ccn(C(F)F)c(=O)c1)NC(=O)OC(C)(C)C] | 451.2 (M + H) |
| 259^{e} | tert-Butyl (2-methyl-1- (5-(2-methylpyrimidin- 5-yl)-3- (trifluoromethyl)-1H- pyrazol-1-yl)propan-2- yl)carbamate | 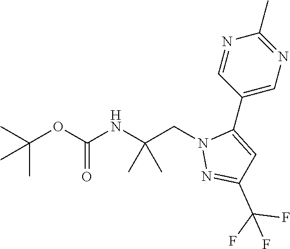 [SMILES: Cc1ncc(-c2cc(C(F)(F)F)nn2CC(C)(C)NC(=O)OC(C)(C)C)cn1] | 422.2 (M + Na) |
| 260^{f} | tert-Butyl (1-(5-(6- methoxypyridazin-3-yl)- 3-(trifluoromethyl)-1H- pyrazol-1-yl)-2- methylpropan-2- yl)carbamate | 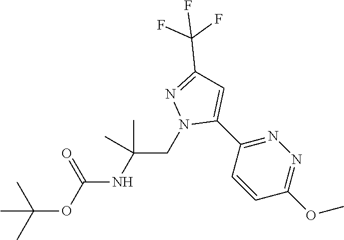 [SMILES: COc1ccc(-c2cc(C(F)(F)F)nn2CC(C)(C)NC(=O)OC(C)(C)C)nn1] | 416.2 (M + H) |
| 261^{g} | tert-Butyl (1-(5-(2-(2- hydroxypropan-2- yl)pyridin-4-yl)-3- (trifluoromethyl)-1H- pyrazol-1-yl)-2- methylpropan-2- yl)carbamate | 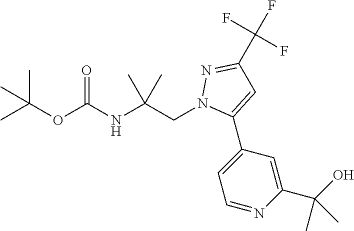 [SMILES: CC(C)(Cn1nc(C(F)(F)F)cc1-c1ccnc(C(C)(C)O)c1)NC(=O)OC(C)(C)C] | 443.4 (M + H) |
| 262^{h} | tert-Butyl (1-(5-(5- hydroxypyridin-2-yl)-3- (trifluoromethyl)-1H- pyrazol-1-yl)-2- methylpropan-2- yl)carbamate | 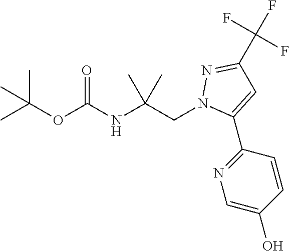 [SMILES: CC(C)(Cn1nc(C(F)(F)F)cc1-c1ccc(O)cn1)NC(=O)OC(C)(C)C] | 401.2 (M + H) |
| 263ⁱ | tert-Butyl (2-methyl-1- (5-(6-(oxetan-3- yl)pyridin-3-yl)-3- (trifluoromethyl)-1H- pyrazol-1-yl)propan-2- yl)carbamate | 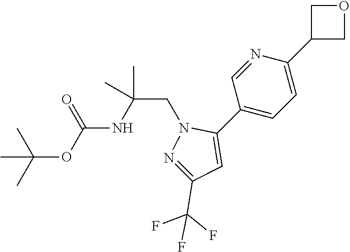 [SMILES: CC(C)(Cn1nc(C(F)(F)F)cc1-c1ccc(C2COC2)nc1)NC(=O)OC(C)(C)C] | 441.2 (M + H) |

^{a}XPhos Pd G3 as catalyst, 10:1 dioxane:water as solvent, purification by column chromatography (SiO₂, gradient 0-3% MeOH in DCM)
^{b}BrettPhos Pd G3 as catalyst, purification by column chromatography (SiO₂, gradient 0-5% MeOH in DCM)
^{c}BrettPhos Pd G3 as catalyst, K₃PO₄ as base, purification by column chromatography (SiO₂, gradient 0-100% EtOAc in heptane)
^{d}BrettPhos Pd G3 as catalyst, purification by column chromatography (SiO₂, gradient 0-24% EtOAc in hexanes)
^{e}BrettPhos Pd G3 as catalyst, purification by column chromatography [SiO₂, gradient 0-50% solvent B (3:1 EtOAc:EtOH) in heptane]
^{f}10:1 dioxane:water as solvent, purification by column chromatography (SiO₂, gradient 10-30% EtOAc in cyclohexane)
^{g}5:1 dioxane:water as solvent, purification by column chromatography (SiO₂, gradient 0-5% MeOH in DCM)
^{h}5:1 dioxane:water as solvent, purification by column chromatography (SiO₂, gradient 0-30% EtOAc in cyclohexane)
ⁱBrettPhos Pd G3 as catalyst, purification by column chromatography [SiO₂, gradient 0-100% solvent B (3:1 EtOAc:EtOH) in heptane]

### Preparation 264

### tert-Butyl (1-(5-(5-fluoro-1-methyl-6-oxo-1,6-dihydropyridin-3-yl)-3-(trifluoromethyl)-1H-pyrazol-1-yl)-2-methylpropan-2-yl)carbamate

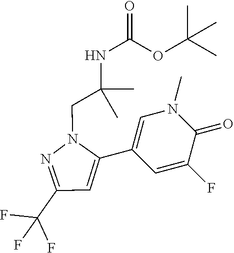 [SMILES: Cn1cc(-c2cc(C(F)(F)F)nn2CC(C)(C)NC(=O)OC(C)(C)C)cc(F)c1=O]

To a mixture of 3-fluoro-1-methyl-5-(4,4,5,5-tetramethyl-1,3,2-dioxaborolan-2-yl)pyridin-2(1H)-one (33.0 g, 95% purity, 124 mmol), tert-butyl (1-(5-bromo-3-(trifluoromethyl)-1H-pyrazol-1-yl)-2-methylpropan-2-yl)carbamate (45.4 g, 118 mmol) and Na₂CO₃ (39.4 g, 372 mmol) in 1,4-dioxane (700 mL) and water (70 mL) was added 1,1′-bis(diphenylphosphino)ferrocene-palladium(II) dichloride (18.1 g, 24.8 mmol). The flask was evacuated and backfilled with N₂×3. The mixture was stirred at 90° C. for 12 h under N₂, filtered, and the filter cake was washed with EtOAc (300 mL×2). The filtrate was concentrated under reduced pressure. Column chromatography (SiO₂, gradient 0-40% EtOAc in petroleum ether) provided the title compound (7.3 g, 12%) as a yellow solid, alongside 33 g impure title compound that was repurified by column chromatography (SiO₂, gradient 0-45% EtOAc in CH₂Cl₂) to provide additional title compound (23.5 g, 42%) as a white solid. ES-MS m/z 433.0 (M+H).

The compounds in the following table were prepared in similar manner as described in Preparation 264 using the appropriate boronic ester or acid. Different reaction temperatures and reaction times can be used. Such variances would be apparent to one skilled in the art.

TABLE 33

| Preparation | | | ES-MS |
|---|---|---|---|
| No. | Chemical Name | Structure | m/z |
| 265 | tert-Butyl (2-methyl-1- (5-methyl-3- (trifluoromethyl)-1H- pyrazol-1-yl)propan-2- yl)carbamate | 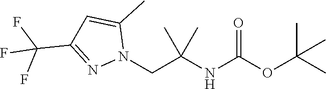 [SMILES: Cc1cc(C(F)(F)F)nn1CC(C)(C)NC(=O)OC(C)(C)C] | 322.2 (M + H) |
| 266* | tert-Butyl (1-(5- cyclopropyl-3- (trifluoromethyl)-1H- pyrazol-1-yl)-2- methylpropan-2- yl)carbamate | 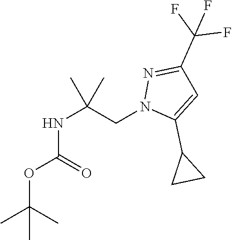 [SMILES: CC(C)(Cn1nc(C(F)(F)F)cc1C1CC1)NC(=O)OC(C)(C)C] | 348.2 (M + H) |

*K₂CO₃ as base, PdCl₂(dtbpf) as catalyst

### Preparation 267

### tert-Butyl (2-methyl-1-(1-(1-methyl-2-oxopyrrolidin-3-yl)-4-(trifluoromethyl)-1H-imidazol-2-yl)propan-2-yl)carbamate

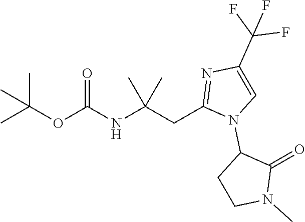 [SMILES: CN1CCC(n2cc(C(F)(F)F)nc2CC(C)(C)NC(=O)OC(C)(C)C)C1=O]

To a 20 mL microwave vial with magnetic stir bar was added tert-butyl (2-methyl-1-(4-(trifluoromethyl)-1H-imidazol-2-yl)propan-2-yl)carbamate (1.06 g, 3.45 mmol) and K₂CO₃ (1.40 g, 10.1 mmol). The vial was capped, evacuated and backfilled with N₂×3, then DMF (9 mL) was added, followed by rac-3-bromo-1-methylpyrrolidin-2-one (760 μL, 6.87 mmol). The reaction mixture was stirred at 60° C. overnight, cooled to RT, and partitioned between EtOAc and H₂O. The layers were separated and the aqueous was extracted with EtOAc×2. The combined organics were washed with saturated aqueous NaCl, dried over Na₂SO₄, filtered, and concentrated under reduced pressure. Column chromatography (SiO₂, gradient 0-100% EtOAc in cyclohexane) provided the title compound (885.7 mg, 64%) as an off-white solid. ES-MS m/z 405.0 (M+H).

The compounds in the following table were prepared in similar manner as described in Preparation 267 using the appropriate alkyl halide. Different reaction temperatures and reaction times can be used. Such variances would be apparent to one skilled in the art.

TABLE 34

| Preparation | | | ES-MS |
|---|---|---|---|
| No. | Chemical Name | Structure | m/z |
| 268 | tert-Butyl (1-(1-(1- cyclopropyl-2- oxopyrrolidin-3-yl)-4- (trifluoromethyl)-1H- imidazol-2-yl)-2- methylpropan-2- yl)carbamate | 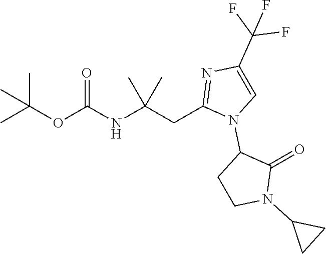 [SMILES: CC(C)(Cc1nc(C(F)(F)F)cn1C1CCN(C2CC2)C1=O)NC(=O)OC(C)(C)C] | 431.2 (M + H) |
| 269 | tert-Butyl (2-methyl-1- (1-(2-(methylamino)-2- oxoethyl)-4- (trifluoromethyl)-1H- imidazol-2-yl)propan-2- yl)carbamate | 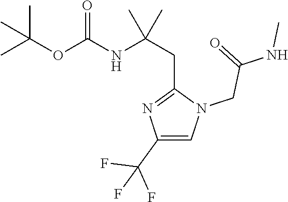 [SMILES: CNC(=O)Cn1cc(C(F)(F)F)nc1CC(C)(C)NC(=O)OC(C)(C)C] | 379.2 (M + H) |
| 270 | tert-Butyl (2-methyl-1- (1-(2-morpholino-2- oxoethyl)-4- (trifluoromethyl)-1H- imidazol-2-yl)propan-2- yl)carbamate | 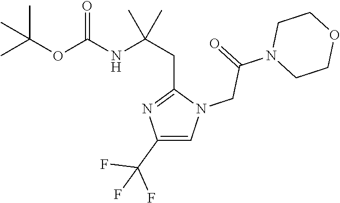 [SMILES: CC(C)(Cc1nc(C(F)(F)F)cn1CC(=O)N1CCOCC1)NC(=O)OC(C)(C)C] | 435.2 (M + H) |
| 271 | tert-Butyl (2-methyl-1- (1-methyl-4- (trifluoromethyl)-1H- imidazol-2-yl)propan-2- yl)carbamate | 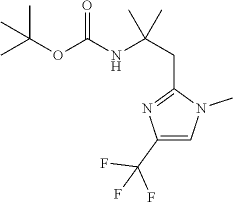 [SMILES: Cn1cc(C(F)(F)F)nc1CC(C)(C)NC(=O)OC(C)(C)C] | 322.1 (M + H) |

### Preparation 272

### tert-Butyl (2-methyl-1-(1-((1-methyl-1H-pyrazol-3-yl)methyl)-4-(trifluoromethyl)-1H-imidazol-2-yl)propan-2-yl)carbamate

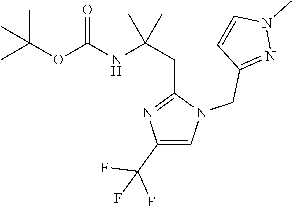 [SMILES: Cn1ccc(Cn2cc(C(F)(F)F)nc2CC(C)(C)NC(=O)OC(C)(C)C)n1]

In a dried 50.0 mL RBF containing a magnetic stir-bar, a mixture of tert-butyl (2-methyl-1-(4-(trifluoromethyl)-1H-imidazol-2-yl)propan-2-yl)carbamate (300.3 mg, 977.2 μmol), (1-methyl-1H-pyrazol-3-yl)methanol (134.5 mg, 98% purity, 1.176 mmol) and triphenylphosphine (513.5 mg, 1.958 mmol) in toluene (10 mL) was degassed and purged with N₂×3, then diisopropyl diazene-1,2-dicarboxylate (390 μL, 1.98 mmol) was added at 0° C. The mixture was stirred at 0° C. for 10 min, heated to 100° C., and stirred for 2 h. The reaction mixture was diluted with H₂O (50 mL) and extracted with EtOAc (70 mL×2). The combined organics were washed with saturated aqueous NaCl (70 mL×2), dried over Na₂SO₄, filtered, and concentrated under reduced pressure. Reversed-phase preparative HPLC (F-Welch Xtimate C18, 40×200 mm, 7 μm, gradient 30-70% ACN in 0.225% aqueous formic acid) provided the title compound (90.2 mg, 22%) as a yellow oil. ES-MS m/z 402.6 (M+H).

### Preparation 273

### tert-Butyl (1-(1-(1-cyclopropyl-2-oxo-1,2-dihydropyridin-4-yl)-4-(trifluoromethyl)-1H-imidazol-2-yl)-2-methylpropan-2-yl)carbamate

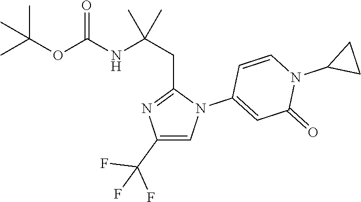 [SMILES: CC(C)(Cc1nc(C(F)(F)F)cn1-c1ccn(C2CC2)c(=O)c1)NC(=O)OC(C)(C)C]

A dried 50 mL RBF containing a magnetic stir bar and a solution of tert-butyl (2-methyl-1-(4-(trifluoromethyl)-1H-imidazol-2-yl)propan-2-yl)carbamate (331 mg, 97.2% purity, 1.05 mmol), (1-cyclopropyl-2-oxo-1,2-dihydropyridin-4-yl)boronic acid (390 mg, 95% purity, 2.07 mmol), 4-dimethylaminopyridine (129 mg, 1.06 mmol), sodium 2-methylpropan-2-olate (306 mg, 3.18 mmol) and 4 Å molecular sieves (517 mg) in DCE (15 mL) was added copper diacetate (193 mg, 1.06 mmol). The mixture was evacuated and backfilled with O₂×3, then stirred at 45° C. for 16 h under O₂ (15 psi). The reaction mixture was filtered, and the filter cake was washed with EtOAc (50 mL). The filtrate was concentrated under reduced pressure. Column chromatography (SiO₂, gradient 0-80% EtOAc in hexane) gave the title compound (141 mg, 24%) as a yellow solid. ES-MS m/z 441.2 (M+H).

### Preparation 274

### tert-Butyl (1-(1-(6-cyanopyridazin-3-yl)-4-(trifluoromethyl)-1H-imidazol-2-yl)-2-methylpropan-2-yl)carbamate

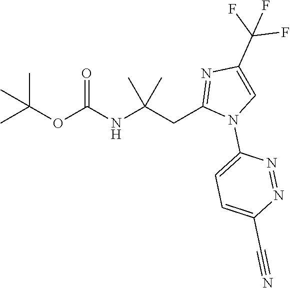 [SMILES: CC(C)(Cc1nc(C(F)(F)F)cn1-c1ccc(C#N)nn1)NC(=O)OC(C)(C)C]

To a microwave vial was added tert-butyl (2-methyl-1-(4-(trifluoromethyl)-1H-imidazol-2-yl)propan-2-yl)carbamate (254 mg, 825 μmol), K₂CO₃ (78.1 mg, 565 μmol) and 6-chloropyridazine-3-carbonitrile (105 mg, 753 μmol). The vial was evacuated and backfilled with N₂×3. Then, DMF (1.72 mL) was added, and the mixture was heated to 80° C. in an aluminum heating block. The reaction was cooled to RT and the mixture was diluted with EtOAc then poured into water. The layers were separated, and the aqueous layer was extracted with EtOAc (×2). The combined organic layers were washed with saturated aqueous NaCl, dried over Na₂SO₄, filtered, and concentrated under reduced pressure. Column chromatography (SiO₂, gradient 0-100% EtOAc in cyclohexane) provided the title compound (231 mg, 65%) as a yellow oil. ES-MS m/z 411.2 (M+H).

### Preparation 275

### 1-(2-((tert-butoxycarbonyl)amino)-2-methylpropyl)-3-(trifluoromethyl)-1H-pyrazole-5-carboxylic acid

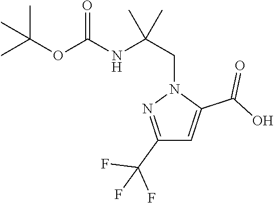 [SMILES: CC(C)(Cn1nc(C(F)(F)F)cc1C(=O)O)NC(=O)OC(C)(C)C]

To a solution of ethyl-1-(2-((tert-butoxycarbonyl)amino)-2-methylpropyl)-3-(trifluoromethyl)-1H-pyrazole-5-carboxylate (500 mg, 1.25 mmol) in a mixture of THF (5 mL) and EtOH (5 mL) was added a solution of lithium hydroxide in water (1.0 M, 6.26 mL, 6.26 mmol). The mixture was stirred at 25° C. for 12 h. The mixture was adjusted to pH 2 with aqueous 1 M HCl, then was extracted with EtOAc (20 mL×2). The combined organic layers were washed with saturated aqueous NaCl (20 mL×2), dried over Na₂SO₄, filtered, and concentrated under reduced pressure to provide the title compound (460 mg, 94%) as a colorless oil. ES-MS m/z 251.8 (M+H-Boc).

### Preparation 276

### tert-Butyl (1-(5-(dimethylcarbamoyl)-3-(trifluoromethyl)-1H-pyrazol-1-yl)-2-methylpropan-2-yl)carbamate

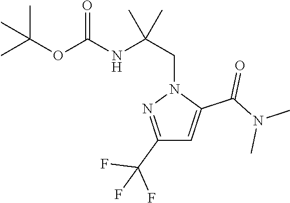 [SMILES: CN(C)C(=O)c1cc(C(F)(F)F)nn1CC(C)(C)NC(=O)OC(C)(C)C]

To a solution of 1-(2-((tert-butoxycarbonyl)amino)-2-methylpropyl)-3-(trifluoromethyl)-1H-pyrazole-5-carboxylic acid (200 mg, 90 wt %, 512 μmol), dimethylamine hydrochloride (85 mg, 1.0 mmol), and HATU (266 mg, 700 μmol) in DMA (3 mL) was added diisopropylethylamine (500 μL, 2.91 mmol). The reaction mixture was stirred at 25° C. for 12 h, then the mixture was diluted with water (50 mL) and extracted with EtOAc (50 mL×2). The combined organic layers were washed with saturated aqueous NaCl (50 mL×3), dried over Na₂SO₄, filtered, and concentrated under reduced pressure. Column chromatography (SiO₂, gradient 0-50% EtOAc in hexanes) provided the title compound (191 mg, 89%) as a white solid. ES-MS m/z 379.0 (M+H).

### Preparation 277

### tert-Butyl (1-(5-([1,2,4]triazolo[4,3-a]pyrimidin-6-yl)-3-(trifluoromethyl)-1H-pyrazol-1-yl)-2-methylpropan-2-yl)carbamate

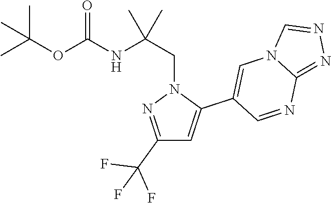 [SMILES: CC(C)(Cn1nc(C(F)(F)F)cc1-c1cnc2nncn2c1)NC(=O)OC(C)(C)C]

In a dry 10-mL RBF, a mixture of tert-butyl (2-methyl-1-(5-(4,4,5,5-tetramethyl-1,3,2-dioxaborolan-2-yl)-3-(trifluoromethyl)-1H-pyrazol-1-yl)propan-2-yl)carbamate (458 mg, 1.00 mmol), 6-bromo-[1,2,4]triazolo[4,3-a]pyrimidine (200 mg, 1.00 mmol), Na₂CO₃ (213 mg, 2.01 mmol) and Pd(dppf)Cl₂ (73.5 mg, 100 μmol) in 1,4-dioxane (5 mL) and water (0.5 mL) was evacuated and backfilled with N₂×3. The mixture was stirred at 70° C. for 12 h under N₂. The reaction mixture was quenched with water (50 mL) then extracted with EtOAc (50 mL×2). The combined organic layers were washed with saturated aqueous NaCl (50 mL×2), dried over Na₂SO₄, filtered, and concentrated under reduced pressure. Column chromatography (SiO₂, gradient 0-50% EtOAc in petroleum ether) provided the product in 89% purity. The material was further purified by prep-HPLC (column: YMC Triart C18 250*30 mm*5 μm; mobile phase: [A: H₂O (10 mM NH₄HCO₃); B: ACN]; B %: 36.00%-66.00%, 10.00 min; flow rate: 25.00 mL/min) to provide the title compound (140 mg, 31%) as a white solid. ES-MS m/z 425.9 (M+H).

### Preparation 278

### tert-Butyl (1-(5-(1-cyclopropyl-2-oxo-1,2-dihydropyrimidin-4-yl)-3-(trifluoromethyl)-1H-pyrazol-1-yl)-2-methylpropan-2-yl)carbamate

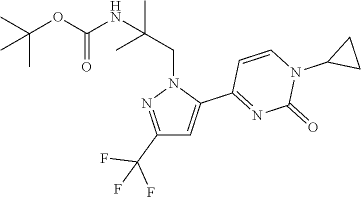 [SMILES: CC(C)(Cn1nc(C(F)(F)F)cc1-c1ccn(C2CC2)c(=O)n1)NC(=O)OC(C)(C)C]

In a dry 50-mL RBF, a solution of 4-chloro-1-cyclopropylpyrimidin-2 (1H)-one (452 mg, 80 wt %, 2.12 mmol) in 1,4-dioxane (20 mL) was treated with tert-butyl (2-methyl-1-(5-(4,4,5,5-tetramethyl-1,3,2-dioxaborolan-2-yl)-3-(trifluoromethyl)-1H-pyrazol-1-yl)propan-2-yl)carbamate (1.15 g, 2.52 mmol), Na₂CO₃ (443 mg, 4.18 mmol), bis(tri-tert-butylphosphine)palladium(0) (226 mg, 442 μmol), and water (2 mL) at 20° C. The reaction mixture was evacuated and backfilled with N₂×3, then the mixture was stirred at 60° C. for 30 minutes under N₂. The reaction mixture was cooled to RT then Na₂SO₄ (˜5 g) was added to the mixture. The suspension was filtered through a short pad of silica gel, washing with EtOAc (100 mL). The filtrate was concentrated under reduced pressure. Column chromatography (SiO₂, gradient 0-90% EtOAc in hexanes) provided the title compound (373 mg, 38%) as a light-yellow solid. ES-MS m/z 442.1 (M+H).

### Preparation 279

### tert-Butyl (1-(2-bromo-4-fluorophenyl)-2-methylpropan-2-yl)carbamate

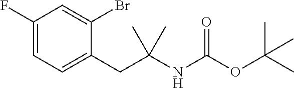 [SMILES: CC(C)(Cc1ccc(F)cc1Br)NC(=O)OC(C)(C)C]

To a solution of 1-(2-bromo-4-fluorophenyl)-2-methylpropan-2-amine (5.01 g, 20.4 mmol) in 1,4-dioxane (50 mL) was added N,N-diisopropylethylamine (7.1 mL, 41 mmol) followed by di-tert-butyl dicarbonate (5.8 g, 6.1 mL, 27 mmol). The reaction mixture was stirred at 17° C. for 16 h, after which it was diluted with H₂O (100 mL) and extracted with EtOAc (2×70 mL). The combined organic layers were washed with saturated aqueous NaCl (2×50 mL), dried over Na₂SO₄, filtered, concentrated under reduced pressure and purified by silica gel chromatography using a gradient of 0-8% EtOAc in hexanes to give the title compound (7.00 g, 93%) as a white solid. ES-MS m/z 289.8, 291.7 (M-isobutene+H).

### Preparation 280

### Butyl 2-(2-((tert-butoxycarbonyl)amino)-2-methylpropyl)-5-fluorobenzoate

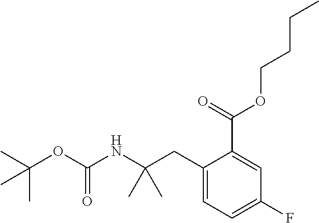 [SMILES: CCCCOC(=O)c1cc(F)ccc1CC(C)(C)NC(=O)OC(C)(C)C]

To a solution of tert-butyl (1-(2-bromo-4-fluorophenyl)-2-methylpropan-2-yl)carbamate (6.00 g, 94 wt %, 16.3 mmol) in DMF (60 mL) was added 1,3-bis(dicyclohexylphosphino)propane bis(tetrafluoroborate) (2.00 g, 3.27 mmol), K₂CO₃ (5.62 g, 40.7 mmol), Pd(OAc)₂ (357 mg, 1.59 mmol) and n-butanol (100 mL). The reaction mixture was degassed and refilled with CO 3×, then placed under an atmosphere of 30 psi CO and stirred at 80° C. for 16 h. Then additional Pd(OAc)₂ (390 mg, 1.74 mmol) was added and the reaction was placed under an atmosphere of 50 psi CO and stirred at 80° C. for an additional 16 h. The reaction was then cooled to RT, diluted with EtOAc (300 mL) and filtered, using additional EtOAc (3×30 mL) to rinse the filter cake. The filtrate was then washed with saturated aqueous NaCl (3×150 mL), dried over Na₂SO₄, filtered, concentrated under reduced pressure, and purified by silica gel chromatography using a gradient of 0-4% EtOAc in hexanes to give the title compound (2.35 g, 38%) as a colorless oil. ES-MS m/z 268.5 (M-Boc+H).

### Preparation 281

### 2-(2-((tert-Butoxycarbonyl)amino)-2-methylpropyl)-5-fluorobenzoic acid

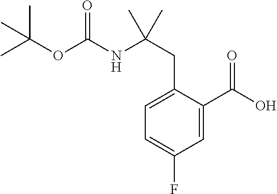 [SMILES: CC(C)(Cc1ccc(F)cc1C(=O)O)NC(=O)OC(C)(C)C]

To a solution of butyl 2-(2-((tert-butoxycarbonyl)amino)-2-methylpropyl)-5-fluorobenzoate (2.35 g, 97 wt %, 6.20 mmol) in THF (30 mL) was added LiOH (32.0 mL of a 2 M aq. soln., 64.0 mmol). Then the reaction mixture was heated to 50° C. and stirred for 16 h. After cooling to RT, the reaction was acidified to pH˜3 using aq. 1 M HCl and extracted with EtOAc (2×60 mL). The combined organic layers were dried over Na₂SO₄, filtered and concentrated under reduced pressure to give the title compound (1.75 g, 86%) as a white solid. ES-MS m/z 212.1 (M-Boc+H).

### Preparation 282

### tert-Butyl (1-(2-amino-4-fluorophenyl)-2-methylpropan-2-yl)carbamate

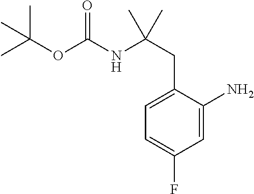 [SMILES: CC(C)(Cc1ccc(F)cc1N)NC(=O)OC(C)(C)C]

To a solution of 2-(2-((tert-butoxycarbonyl)amino)-2-methylpropyl)-5-fluorobenzoic acid (201 mg, 95 wt %, 613 μmol) in DMF (2 mL) was added TEA (215 μL, 1.54 mmol) followed by diphenylphosphoryl azide (197 μL, 913 μmol). Stirring was initiated and the reaction was heated to 90° C. for 2 h. Then H₂O (0.5 mL) was added, and the mixture was stirred at 90° C. for 16 h, then 100° C. for 3 h, then 130° C. for 4 h, after which the reaction was cooled to RT, diluted with H₂O (10 mL) and extracted with EtOAc (2×10 mL). The combined organic layers were washed with saturated aqueous NaCl (2×15 mL), dried over Na₂SO₄, filtered, concentrated under reduced pressure and purified by silica gel chromatography using a gradient of 0-12% EtOAc in hexanes to give the title compound (136 mg, 71%) as a colorless oil. ES-MS m/z 227.2 (M-isobutene+H).

### Preparation 283

### tert-Butyl (1-(2-(3-(2-chloroacetyl)ureido)-4-fluorophenyl)-2-methylpropan-2-yl)carbamate

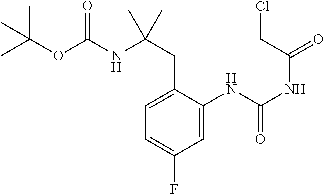 [SMILES: CC(C)(Cc1ccc(F)cc1NC(=O)NC(=O)CCl)NC(=O)OC(C)(C)C]

A solution of tert-butyl (1-(2-amino-4-fluorophenyl)-2-methylpropan-2-yl)carbamate (136 mg, 91 wt %, 438 μmol) in THF (3 mL) was degassed and refilled with N₂ 3×. The mixture was then cooled to −40° C. and a solution of 2-chloroacetyl isocyanate (65 μL, 0.76 mmol) in THF (1 mL) was added dropwise. The reaction mixture was allowed to stir at −40° C. for 1 h, after which it was quenched at that temperature by dilution with EtOAc (5 mL) and sat. aq. NaHCO₃ (5 mL). The mixture was then allowed to warm to RT, and the organic layer was removed. The aqueous layer was extracted with EtOAc (2×5 mL) and then the combined organic layers were washed with saturated aqueous NaCl (10 mL), dried over Na₂SO₄, filtered, concentrated under reduced pressure, and purified by silica gel chromatography using a gradient of 0-18% EtOAc in hexanes to give the title compound (161 mg, 90%) as a white solid. ES-MS m/z 302.2, 304.0 (M-Boc+H).

### Preparation 284

### tert-Butyl (1-(2-(2,4-dioxoimidazolidin-1-yl)-4-fluorophenyl)-2-methylpropan-2-yl)carbamate

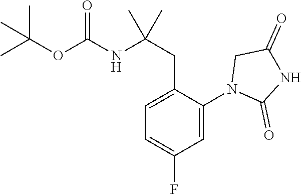 [SMILES: CC(C)(Cc1ccc(F)cc1N1CC(=O)NC1=O)NC(=O)OC(C)(C)C]

A mixture of tert-butyl (1-(2-(3-(2-chloroacetyl)ureido)-4-fluorophenyl)-2-methylpropan-2-yl)carbamate (161 mg, 98% wt, 0.393 mmol) in DMF (4 mL) was degassed and refilled with N₂×3. Then NaH (34 mg, 60% wt, 0.85 mmol) was added in one portion. The reaction mixture was stirred at RT for 3 h. The mixture was quenched by the addition of sat. aq. NH₄Cl (20 mL) and extracted with EtOAc (20 mL×2). The combined organic layer was washed with saturated aqueous NaCl (20 mL×3), dried over anhydrous Na₂SO₄, filtered and concentrated under reduced pressure. The residue was purified by column chromatography (SiO₂, 0-3% MeOH/DCM gradient) to give the title compound (97 mg, 63%) as a white solid. ES/MS m/z 266.2 (M-Boc+H).

### Preparation 285

### (2-(2-((tert-Butoxycarbonyl)amino)-2-methylpropyl)-5-fluorophenyl)boronic acid

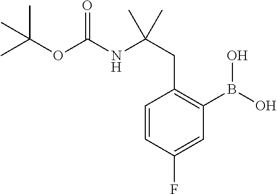 [SMILES: CC(C)(Cc1ccc(F)cc1B(O)O)NC(=O)OC(C)(C)C]

To a solution of tert-butyl (1-(2-bromo-4-fluorophenyl)-2-methylpropan-2-yl)carbamate (20.0 g, 95% wt, 54.9 mmol) in MeOH (200 mL) was added potassium acetate (13.5 g, 8.58 mL, 137 mmol) and ethylene glycol (10.2 g, 9.18 mL, 165 mmol). The mixture was purged and backfilled with N₂×3. Hypodiboric acid (9.84 g, 110 mmol) and chloro[(di(1-adamantyl)-N-butylphosphine)-2-(2-aminobiphenyl)]palladium(II) (183 mg, 274 μmol) were added. The mixture was purged and backfilled with N₂×3. The reaction mixture was stirred at 35° C. for 20 h. The reaction was filtered, washing with MeOH (100 mL). The filtrate (combined with another reaction batch of 19.5 g tert-butyl (1-(2-bromo-4-fluorophenyl)-2-methylpropan-2-yl)carbamate) was concentrated under reduced pressure. The residue was poured into water (300 mL) and extracted with EtOAc (300 mL×3). The combined organic layer was dried over anhydrous Na₂SO₄, filtered and concentrated under reduced pressure. The residue was purified by column chromatography (SiO₂, 0-20% EtOAc/petroleum ether gradient) to give the title compound (28.5 g, 75%) as a dark brown oil. ES/MS m/z 312.0 (M+H)

### Preparation 286

### tert-Butyl (1-(4-fluoro-2-(1-methyl-6-oxo-1,6-dihydropyrimidin-4-yl)phenyl)-2-methylpropan-2-yl)carbamate

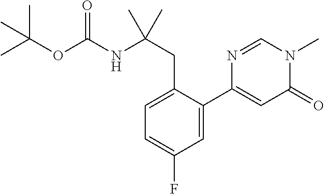 [SMILES: Cn1cnc(-c2cc(F)ccc2CC(C)(C)NC(=O)OC(C)(C)C)cc1=O]

A mixture of (2-(2-((tert-butoxycarbonyl)amino)-2-methylpropyl)-5-fluorophenyl)boronic acid (366 mg, 1.06 mmol), 6-bromo-3-methylpyrimidin-4 (3H)-one (200 mg, 1.06 mmol), sodium carbonate (336 mg, 3.17 mmol) and XPhos Pd G3 (179 mg, 212 μmol) in 1,4-dioxane (5 mL) and water (0.5 mL) was degassed and refilled with N₂×3. The mixture was stirred at 85° C. for 12 h. After cooling to RT, the reaction was quenched by the addition of H₂O (30 mL) and extracted with EtOAc (30 mL×2). The combined organic layer was washed with saturated aqueous NaCl (30 mL×2), dried over anhydrous Na₂SO₄, filtered and concentrated under reduced pressure. The residue was purified by column chromatography (SiO₂, 0-30% EtOAc/hexanes gradient) to give the title compound (281 mg, 64%) as a white solid. ES/MS m/z 376.0 (M+H)

### Preparation 287

### 4-(1-(2-Amino-2-methylpropyl)-3-(trifluoromethyl)-1H-pyrazol-5-yl)-1-cyclopropylpyridin-2 (1H)-one hydrochloride

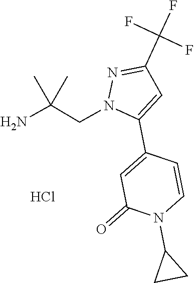 [SMILES: CC(C)(N)Cn1nc(C(F)(F)F)cc1-c1ccn(C2CC2)c(=O)c1 ; Cl]

To tert-butyl (1-(5-(1-cyclopropyl-2-oxo-1,2-dihydropyridin-4-yl)-3-(trifluoromethyl)-1H-pyrazol-1-yl)-2-methylpropan-2-yl)carbamate (155 mg, 352 μmol) in DCM (1.0 mL) was added hydrogen chloride in dioxane (460 μL, 4 molar, 1.84 mmol). The reaction was stirred at RT for 19 h. The reaction was concentrated under a stream of N₂ and taken forward assuming quantitative yield without further purification. ES/MS m/z 341.0

The compounds in the following table were prepared in similar manner as described in Preparation 287 using the appropriate carbamate. Different reaction temperatures and reaction times can be used. Such variances would be apparent to one skilled in the art.

TABLE 35

| Preparation | | | ES-MS |
|---|---|---|---|
| No. | Chemical Name | Structure | m/z |
| 288 | 5-(1-(2-Amino-2- methylpropyl)-3- (trifluoromethyl)-1H- pyrazol-5-yl)-1-(methyl- d3)pyridin-2(1H)-one hydrochloride | 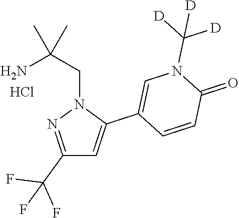 [SMILES: [2H]C([2H])([2H])n1cc(-c2cc(C(F)(F)F)nn2CC(C)(C)N)ccc1=O ; Cl] | 318.1 (M + H) |
| 289 | 1-(5-(2- ((Dimethylamino)methyl) pyrimidin-5-yl)-3- (trifluoromethyl)-1H- pyrazol-1-yl)-2- methylpropan-2-amine dihydrochloride | 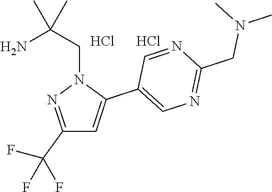 [SMILES: CN(C)Cc1ncc(-c2cc(C(F)(F)F)nn2CC(C)(C)N)cn1 ; Cl ; Cl] | 342.9 (M + H) |
| 290 | 4-(1-(2-Amino-2- methylpropyl)-3- (trifluoromethyl)-1H- pyrazol-5-yl)-1- cyclobutylpyridin- 2(1H)-one hydrochloride | 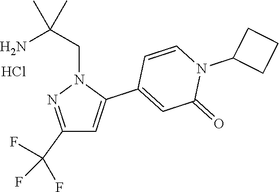 [SMILES: Cl ; CC(C)(N)Cn1nc(C(F)(F)F)cc1-c1ccn(C2CCC2)c(=O)c1] | 355.2 (M + H) |
| 291 | 4-(1-(2-Amino-2- methylpropyl)-3- (trifluoromethyl)-1H- pyrazol-5-yl)-1- (difluoromethyl)pyridin- 2(1H)-one hydrochloride | 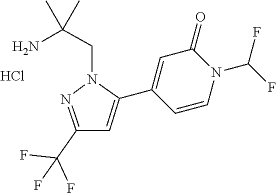 [SMILES: Cl ; CC(C)(N)Cn1nc(C(F)(F)F)cc1-c1ccn(C(F)F)c(=O)c1] | 351.1 (M + H) |
| 292 | 2-Methyl-1-(5-(2- methylpyrimidin-5-yl)- 3-(trifluoromethyl)-1H- pyrazol-1-yl)propan-2- amine hydrochloride | 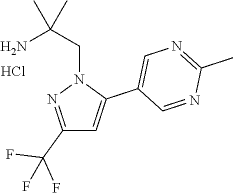 [SMILES: Cl ; Cc1ncc(-c2cc(C(F)(F)F)nn2CC(C)(C)N)cn1] | 300.2 (M + H) |
| 293 | 1-(5-(6- Methoxypyridazin-3-yl)- 3-(trifluoromethyl)-1H- pyrazol-1-yl)-2- methylpropan-2-amine dihydrochloride | 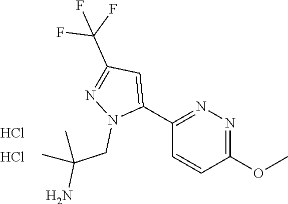 [SMILES: Cl ; Cl ; COc1ccc(-c2cc(C(F)(F)F)nn2CC(C)(C)N)nn1] | 315.8 (M + H) |
| 294 | 2-(4-(1-(2-Amino-2- methylpropyl)-3- (trifluoromethyl)-1H- pyrazol-5-yl)pyridin-2- yl)propan-2-ol hydrochloride | 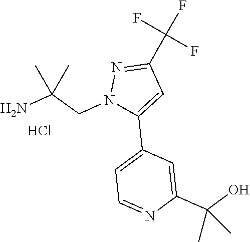 [SMILES: CC(C)(N)Cn1nc(C(F)(F)F)cc1-c1ccnc(C(C)(C)O)c1 ; Cl] | 343.0 (M + H) |
| 295 | 6-(1-(2-Amino-2- methylpropyl)-3- (trifluoromethyl)-1H- pyrazol-5-yl)pyridin-3- ol hydrochloride | 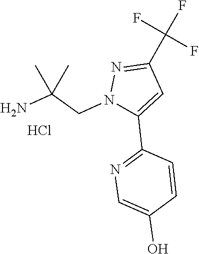 [SMILES: CC(C)(N)Cn1nc(C(F)(F)F)cc1-c1ccc(O)cn1 ; Cl] | 301.0 (M + H) |
| 296 | 1-(5-Methoxy-3- (trifluoromethyl)-1H- pyrazol-1-yl)-2- methylpropan-2-amine hydrochloride | 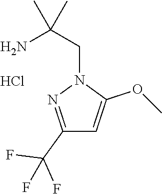 [SMILES: Cl ; COc1cc(C(F)(F)F)nn1CC(C)(C)N] | 238.2 (M + H) |
| 297 | 1-(2-Amino-2- methylpropyl)-N,N- dimethyl-3- (trifluoromethyl)-1H- pyrazole-5-carboxamide hydrochloride | 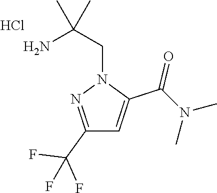 [SMILES: Cl ; CN(C)C(=O)c1cc(C(F)(F)F)nn1CC(C)(C)N] | 278.9 (M + H) |
| 298 | 1-(5-Bromo-3- (trifluoromethyl)-1H- pyrazol-1-yl)-2- methylpropan-2-amine hydrochloride | 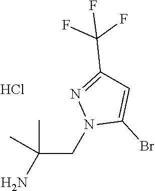 | 286.2 (M + H) |
| 299 | 5-(1-(2-Amino-2- methylpropyl)-3- (trifluoromethyl)-1H- pyrazol-5-yl)-3-fluoro-1- methylpyridin-2(1H)- one hydrochloride | 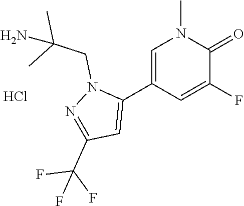 [SMILES: Cl ; Cn1cc(-c2cc(C(F)(F)F)nn2CC(C)(C)N)cc(F)c1=O] | 332.9 (M + H) |
| 300 | 2-Methyl-1-(5-methyl-3- (trifluoromethyl)-1H- pyrazol-1-yl)propan-2- amine hydrochloride | 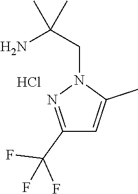 | 222.2 (M + H) |
| 301 | 1-(5-Cyclopropyl-3- (trifluoromethyl)-1H- pyrazol-1-yl)-2- methylpropan-2-amine dihydrochloride | 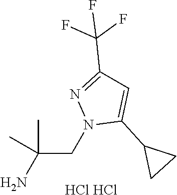 | 248.2 (M + H) |
| 302 | 1-(5- ([1,2,4]Triazolo[4,3- a]pyrimidin-6-yl)-3- (trifluoromethyl)-1H- pyrazol-1-yl)-2- methylpropan-2-amine hydrochloride | 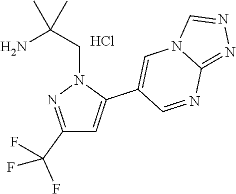 [SMILES: CC(C)(N)Cn1nc(C(F)(F)F)cc1-c1cnc2nncn2c1 ; Cl] | 326.0 (M + H) |
| 303 | 4-(1-(2-Amino-2- methylpropyl)-3- (trifluoromethyl)-1H- pyrazol-5-yl)-1- cyclopropylpyrimidin- 2(1H)-one hydrochloride | 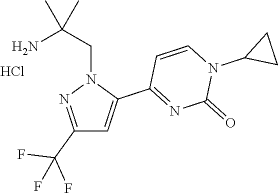 [SMILES: Cl ; CC(C)(N)Cn1nc(C(F)(F)F)cc1-c1ccn(C2CC2)c(=O)n1] | 342.1 (M + H) |
| 304 | 3-(2-(2-Amino-2- methylpropyl)-4- (trifluoromethyl)-1H- imidazol-1-yl)-1- methylpyrrolidin-2-one hydrochloride (racemic mixture) | 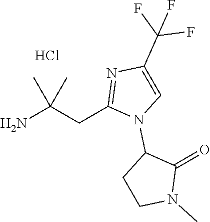 [SMILES: CN1CCC(n2cc(C(F)(F)F)nc2CC(C)(C)N)C1=O ; Cl] | 305.0 (M + H) |
| 305 | 2-Methyl-1-(1-((1- methyl-1H-pyrazol-3- yl)methyl)-4- (trifluoromethyl)-1H- imidazol-2-yl)propan-2- amine dihydrochloride | 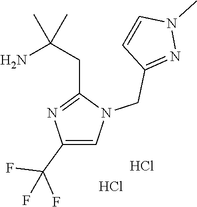 [SMILES: Cn1ccc(Cn2cc(C(F)(F)F)nc2CC(C)(C)N)n1 ; Cl ; Cl] | 302.3 (M + H) |
| 306 | 3-(2-(2-Amino-2- methylpropyl)-4- (trifluoromethyl)-1H- imidazol-1-yl)-1- cyclopropylpyrrolidin-2- one hydrochloride (racemic mixture) | 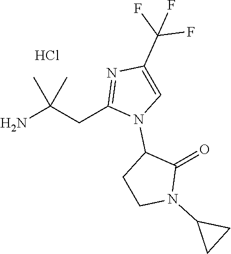 [SMILES: CC(C)(N)Cc1nc(C(F)(F)F)cn1C1CCN(C2CC2)C1=O ; Cl] | 331.2 (M + H) |
| 307 | 4-(2-(2-Amino-2- methylpropyl)-4- (trifluoromethyl)-1H- imidazol-1-yl)-1- cyclopropylpyridin- 2(1H)-one hydrochloride | 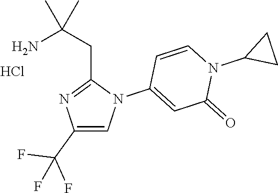 [SMILES: Cl ; CC(C)(N)Cc1nc(C(F)(F)F)cn1-c1ccn(C2CC2)c(=O)c1] | 341.1 (M + H) |
| 308 | 2-(2-(2-Amino-2- methylpropyl)-4- (trifluoromethyl)-1H- imidazol-1-yl)-N- methylacetamide hydrochloride | 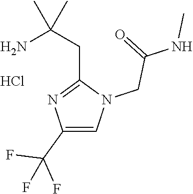 [SMILES: Cl ; CNC(=O)Cn1cc(C(F)(F)F)nc1CC(C)(C)N] | 279.0 (M + H) |
| 309 | 2-(2-(2-Amino-2- methylpropyl)-4- (trifluoromethyl)-1H- imidazol-1-yl)-1- morpholinoethan-1-one hydrochloride | 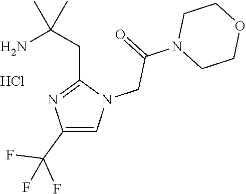 [SMILES: Cl ; CC(C)(N)Cc1nc(C(F)(F)F)cn1CC(=O)N1CCOCC1] | 335.2 (M + H) |
| 310 | 2-Methyl-1-(1-methyl-4- (trifluoromethyl)-1H- imidazol-2-yl)propan-2- amine dihydrochloride | 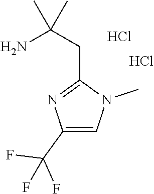 | 222.1 (M + H) |
| 311 | 6-(2-(2-Amino-2- methylpropyl)-4- (trifluoromethyl)-1H- imidazol-1- yl)pyridazine-3- carbonitrile hydrochloride | 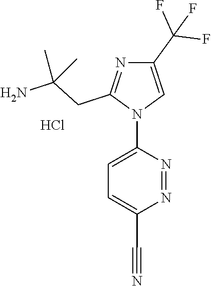 [SMILES: CC(C)(N)Cc1nc(C(F)(F)F)cn1-c1ccc(C#N)nn1 ; Cl] | 311.0 (M + H) |
| 312 | 1-(2-(2-Amino-2- methylpropyl)-5- fluorophenyl) imidazolidine- 2,4-dione hydrochloride | 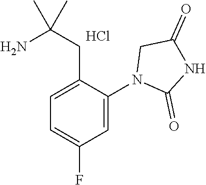 [SMILES: CC(C)(N)Cc1ccc(F)cc1N1CC(=O)NC1=O ; Cl] | 266.0 (M + H) |
| 313 | 6-(2-(2-Amino-2- methylpropyl)-5- fluorophenyl)-3- methylpyrimidin-4(3H)- one hydrochloride | 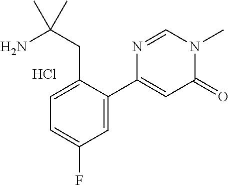 [SMILES: Cn1cnc(-c2cc(F)ccc2CC(C)(C)N)cc1=O ; Cl] | 275.9 (M + H) |

### Preparation 314

### 6-(1-(2-Amino-2-methylpropyl)-3-(trifluoromethyl)-1H-pyrazol-5-yl)pyridazin-3 (2H)-one dihydrochloride

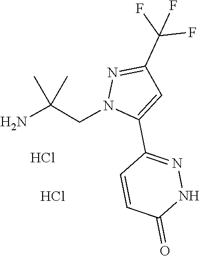 [SMILES: CC(C)(N)Cn1nc(C(F)(F)F)cc1-c1ccc(=O)[nH]n1 ; Cl ; Cl]

To a solution of (300 mg, 1 eq, 773 μmol) in 1,4-dioxane (2 mL) under an N₂ atmosphere was added hydrogen chloride in dioxane (2 mL, 4 molar, 7.73 mmol). The mixture was stirred at 40° C. overnight. The following morning, the reaction was cooled to RT and additional hydrogen chloride in dioxane (2 mL, 4 molar, 7.73 mmol) was added. The mixture was stirred at 40° C. over the weekend. The reaction was concentrated under reduced pressure to give the title compound as a white solid. Assumed quantitative yield and material was taken forward without further purification. ES/MS m/z 302.2 (M+H)

### Preparation 315

### 2-Methyl-1-(5-(6-(oxetan-3-yl)pyridin-3-yl)-3-(trifluoromethyl)-1H-pyrazol-1-yl)propan-2-amine

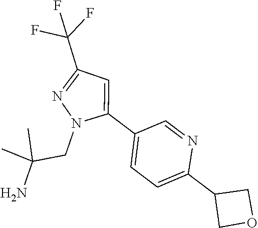 [SMILES: CC(C)(N)Cn1nc(C(F)(F)F)cc1-c1ccc(C2COC2)nc1]

To a mixture of tert-butyl (2-methyl-1-(5-(6-(oxetan-3-yl)pyridin-3-yl)-3-(trifluoromethyl)-1H-pyrazol-1-yl)propan-2-yl)carbamate (44.0 mg, 1 eq, 100 μmol) in DCM (1.0 mL) was added TFA (222 mg, 150 μL, 19.5 eq, 1.95 mmol) at 0° C. The reaction was stirred for 2 h before being diluted with DCM and quenched with sat. aq. Na₂CO₃. The layers were separated and the aqueous layer was extracted with DCM (×3). The combined organic layer was dried over anhydrous Na₂SO₄, filtered and concentrated under reduced pressure to afford the title compound (39.2 mg, 93% purity, 110%) as a pale yellow solid. ES/MS m/z 341.2 (M+H)

### Preparation 316

### 1-(Methyl-d3)-1H-pyrazole-4-carbaldehyde

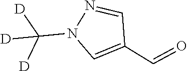 [SMILES: [2H]C([2H])([2H])n1cc(C=O)cn1]

To a solution of 1H-pyrazole-4-carbaldehyde (10.1 g, 1 Eq, 105 mmol) and cesium carbonate (33.6 g, 0.981 Eq, 103 mmol) in DMF (200 mL) under N₂ atmosphere was added iodomethane-d₃ (18.0 g, 7.73 mL, 1.18 Eq, 124 mmol). The reaction was stirred at 60° C. for 20 h. The reaction was cooled to RT and then poured into a separatory funnel with H₂O (400 mL) and EtOAc (250 mL). The layers were separated and the aqueous layer was washed with EtOAc (250 mL). The combined organic layer was washed with saturated aqueous NaCl (50 mL×3), dried over anhydrous Na₂SO₄, filtered and concentrated under reduced pressure. The residue was purified by column chromatography (SiO₂, 20-80% EtOAc/hexane gradient) to give the title compound (4.06 g, 34% yield) as a colorless oil. ES/MS m/z 114.2 (M+H)

### Preparation 317

### Methyl (Z)-2-((tert-butoxycarbonyl)amino)-3-(1-(methyl-d3)-1H-pyrazol-4-yl)acrylate

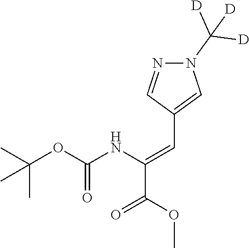 [SMILES: [2H]C([2H])([2H])n1cc(/C=C(\NC(=O)OC(C)(C)C)C(=O)OC)cn1]

To a solution of 1-(methyl-d₃)-1H-pyrazole-4-carbaldehyde (4.0 g, 1 eq, 35 mmol) and methyl 2-{[(tert-butoxy)carbonyl]amino}-2-(dimethoxyphosphoryl)acetate (12.0 g, 1.1 Eq, 40.4 mmol) in DCM (48 mL) under N₂ atmosphere was added DBU (6.1 g, 6.0 mL, 1.1 Eq, 40 mmol) dropwise such that the internal temperature did not exceed 30° C. The reaction was stirred at RT overnight at which point it was added to a separatory funnel with DCM (30 mL). The organic layer was washed with water (50 mL), sat. aq. (NH₄)₂SO₄ (50 mL) and aq. sat. K₂CO₃. The organic layer was dried over anhydrous Na₂SO₄, filtered and concentrated under reduced pressure. The residue was purified by column chromatography (SiO₂, 50-100% EtOAc/hexane gradient) to give a colorless foam which was dissolved in DCM (15 mL) and heptane (20 mL). The mixture was concentrated under reduced pressure to give the title compound (7.56 g, 75% yield) as a white solid. ES/MS m/z 285.4 (M+H)

### Preparation 318

### Methyl (R)-2-((tert-butoxycarbonyl)amino)-3-(1-(methyl-d3)-1H-pyrazol-4-yl)propanoate

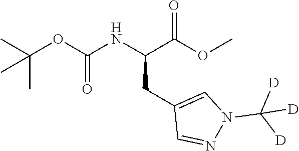 [SMILES: [2H]C([2H])([2H])n1cc(C[C@@H](NC(=O)OC(C)(C)C)C(=O)OC)cn1]

In a Parr stirred autoclave under N₂ atmosphere was added methyl (Z)-2-((tert-butoxycarbonyl)amino)-3-(1-(methyl-d₃)-1H-pyrazol-4-yl)acrylate (5.71 g, 1 eq, 20.1 mmol), Rh-COD-[(R)-MaxPhos]-BF₄ (612 mg, 0.05 eq, 1.09 mmol) and 2,2,2-trifluoroethanol (80 mL). The autoclave was purged with H₂ and pressurized to 200 psi. The reaction was stirred at 40° C. for 5 days. The reaction was concentrated under reduced pressure. The residue was purified by column chromatography (SiO₂, 75-100% EtOAc/hexane gradient) to afford the title compound (4.82 g, 84% yield) as an off-white solid. Material was analyzed by chiral LC (Lux 5 mm i-Amylose-3, 4.6×100 mm column eluted with 15% IPA (0.2% IPAm)/CO₂ at 5 mL/min and detecting at 225 nm) showing 92% ee of the faster eluting isomer. ES-MS m/z 287.4 (M+H)

### Preparation 319

### Lithium (R)-2-((tert-butoxycarbonyl)amino)-3-(1-(methyl-d3)-1H-pyrazol-4-yl)propanoate

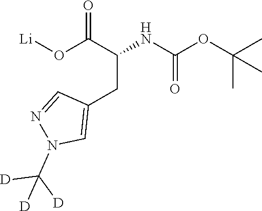

A 40 mL vial was charged with methyl (R)-2-((tert-butoxycarbonyl)amino)-3-(1-(methyl-d₃)-1H-pyrazol-4-yl)propanoate (1.18 g, 4.12 mmol) and t-BuOH (8 mL), along with a stir bar. The vial was capped under air and sonicated until a homogeneous solution was obtained. The reaction mixture was then stirred at 0° C. for 5 min, after which aqueous lithium hydroxide (0.13 g, 2.7 mL, 2M, 5.4 mmol) was added dropwise. Upon complete addition, the reaction mixture was removed from the ice bath and allowed to warm to RT, stirring for 1 h. The reaction was then quenched with aqueous hydrochloric acid (58 mg, 1.6 mL, 1M, 1.6 mmol) and lyophilized to afford the title compound as a white foam (1.11 g, 3.99 mmol, 97%). ES/MS m/z: 273.2 (M+H).

### Preparation 320

### tert-Butyl (R)-(1-((2-methyl-1-(5-(2-methylpyrimidin-5-yl)-3-(trifluoromethyl)-1H-pyrazol-1-yl)propan-2-yl)amino)-3-(1-(methyl-d3)-1H-pyrazol-4-yl)-1-oxopropan-2-yl)carbamate

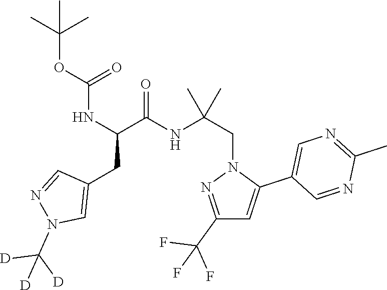 [SMILES: [2H]C([2H])([2H])n1cc(C[C@@H](NC(=O)OC(C)(C)C)C(=O)NC(C)(C)Cn2nc(C(F)(F)F)cc2-c2cnc(C)nc2)cn1]

A solution of lithium (R)-2-((tert-butoxycarbonyl)amino)-3-(1-(methyl-d3)-1H-pyrazol-4-yl)propanoate (1.00 g, 1 eq, 3.59 mmol) and 2-methyl-1-(5-(2-methylpyrimidin-5-yl)-3-(trifluoromethyl)-1H-pyrazol-1-yl)propan-2-amine, HCl (1.31 g, 1.09 eq, 3.90 mmol) in DMSO (5 mL) was sonicated until most of the solids were dissolved. Then, TEA (0.73 g, 1.0 mL, 2.0 eq, 7.2 mmol) and HATU (1.68 g, 1.23 eq, 4.42 mmol) were added and the reaction was stirred at RT under air for 30 min. The reaction mixture was quenched with water (5 mL), loaded onto a diatomaceous earth cartridge and purified by reverse phase chromatography (C18, 0-100% H₂O (0.1% FA)/ACN gradient) to afford the title compound (1.97 g, 99% purity, 98% yield) as a pink foam. ES-MS m/z 554.4 (M+H)

### Preparation 321

### Methyl (Z)-2-((tert-butoxycarbonyl)amino)-3-(imidazo[1,2-a]pyrimidin-3-yl)acrylate

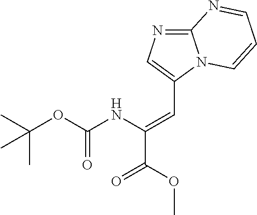 [SMILES: COC(=O)/C(=C/c1cnc2ncccn12)NC(=O)OC(C)(C)C]

A 250 mL three-necked RBF, equipped with a magnetic stirrer, addition funnel and thermometer, was charged with imidazo[1,2-a]pyrimidine-3-carbaldehyde (10.00 g, 67.9 mmol), dissolved in DCM (50 mL). Methyl 2-(dimethoxyphosphoryl)-2-((isopropoxycarbonyl)amino)acetate (23.10 g, 81.6 mmol) was added to the flask. DBU (11.38 g, 11.27 mL, 74.8 mmol) was then added dropwise via the addition funnel at 15° C. The reaction mixture was stirred at 20° C. for 16 h. After completion, the reaction was extracted with water. The organic phase was separated, dried over Na₂SO₄, and filtered. The filtrate was concentrated under vacuum to afford the crude product. The crude material was triturated with DCM, filtered, and the title compound (14 g, 44 mmol, 64%, 99% purity) was obtained as yellow solid. ¹H-NMR (400 MHz, DMSO): 8.55 (1H, J=2.4 Hz, d), 8.44 (1H, J=2.4 Hz, d), 7.96 (1H, s), 7.37 (1H, J=8.4 Hz, d), 4.30-4.28 (1H, m), 3.65 (3H, s), 3.12-3.08 (1H, m), 2.51-2.50 (1H, m), 1.31 (9H, s).

### Preparation 322

### Methyl (R)-2-((tert-butoxycarbonyl)amino)-3-(imidazo[1,2-a]pyrimidin-3-yl)propanoate

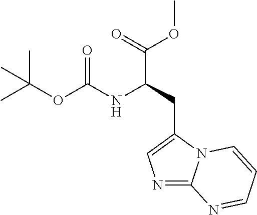 [SMILES: COC(=O)[C@@H](Cc1cnc2ncccn12)NC(=O)OC(C)(C)C]

In a glove box under nitrogen, methyl (Z)-2-((tert-butoxycarbonyl)amino)-3-(imidazo[1,2-a]pyrimidin-3-yl)acrylate (20.84 g, 65.47 mmol) and Rh-COD-[(R)-MaxPhos]-BF₄ (770 mg, 1.37 mmol) were added to a 600 mL Parr stirred autoclave. Degassed 1,1,1,3,3,3-hexafluoroisopropanol (33 mL) and 2,2,2-trifluoroethanol (367 mL) were then added. The autoclave was sealed, removed from the glove box, purged with hydrogen, and pressurized to 250 psi with H₂. The temperature was gradually raised to 60° C. and stirred for 5 h. Afterward, the autoclave was vented, and the heat was turned off. The autoclave was purged with nitrogen and stirred slowly. It was then transferred into a tared 1 L flask, with EtOAc used to rinse the autoclave. The solvent was removed by rotary evaporation, and the resulting residue was recrystallized with MTBE. The solids were collected and washed with 100 mL of cold MTBE. Finally, the solids were dried under air flow on the filter to yield the title compound (9.30 g, 44%) as a tan solid with ee >99%. ES/MS m/z: 321.4 (M+H).

### Preparation 323

### Methyl (R)-2-((tert-butoxycarbonyl)amino)-3-(6-methylpyridin-3-yl)propanoate

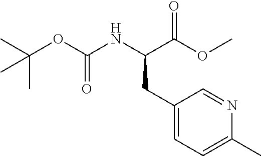 [SMILES: COC(=O)[C@@H](Cc1ccc(C)nc1)NC(=O)OC(C)(C)C]

The reaction was conducted in a dried 100 mL three-neck flask equipped with a magnetic stir bar. To the flask, a mixture of 5-iodo-2-methylpyridine (3.00 g, 13.7 mmol), methyl(S)-2-((tert-butoxycarbonyl)amino)-3-iodopropanoate (6.77 g, 20.6 mmol), nickel chloride-dimethoxyethane adduct (605 mg, 2.75 mmol), and pyridine-2,6-bis(carboximidamide)dihydrochloride (721 mg, 90% wt, 2.75 mmol) in DMA (40 mL) was added zinc (1.80 g, 27.5 mmol). The mixture was degassed, purged with N₂ three times, and stirred at 40° C. for 16 h. After completion, the mixture was diluted with EtOAc (50 mL) and filtered. The filter cake was washed with EtOAc (50 mL). The filtrate was then diluted with H₂O (100 mL) and extracted with EtOAc (100 mL×3). The combined organic layers were washed with saturated aqueous NaCl (100 mL×3), dried over anhydrous Na₂SO₄, filtered, and concentrated under reduced pressure to yield a residue. The filter cake was quenched by adding 1 N HCl (30 mL). The residue was purified by flash silica gel chromatography (Eluent of 0˜40% EtOAc/hexanes gradient) to give the title compound (2.80 g, 9.3 mmol, 68%, 98% purity) as a colorless oil. ES/MS m/z: 295.2 (M+H).

### Preparation 324

### Methyl (R)-2-((tert-butoxycarbonyl)amino)-3-(isothiazol-4-yl)propanoate

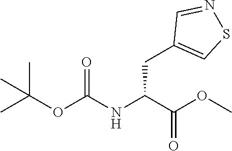 [SMILES: COC(=O)[C@@H](Cc1cnsc1)NC(=O)OC(C)(C)C]

To a solution of 4-bromoisothiazole (5.0 g, 30 mmol) and(S)-(2-((tert-butoxycarbonyl)amino)-3-methoxy-3-oxopropyl)zinc(II)iodide (CAS 2467738-31-2, 23 g, 57 mmol) in DMF (50 mL), tris(dibenzylideneacetone)dipalladium (2.8 g, 3.0 mmol) and 2-dicyclohexylphosphino-2,6-dimethoxy-1,1-biphenyl (1.3 g, 3.0 mmol) were added. The mixture was stirred at 65° C. for 16 h under nitrogen. After completion, the reaction mixture was filtered and concentrated under reduced pressure to yield a residue. The filter cake was quenched with saturated NH₄Cl (aq), and the residue was diluted with water (300 mL) and extracted with EtOAc (300 mL×3). The combined organic layers were washed with saturated aqueous NaCl (300 mL×2), dried over Na₂SO₄, filtered, and concentrated under reduced pressure to give a residue. The residue was purified by flash silica gel chromatography (Eluent of 0˜5% DCM/MeOH gradient) to provide the title compound (10.3 g, 34 mmol, 110%, 95% purity) as a brown oil. ES/MS m/z: 286.9 (M+H).

### Preparation 325

### 3-Bromo-8-methoxyimidazo[1,2-a]pyrazine

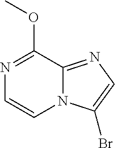

To a solution of 3-bromo-8-chloro-imidazo[1,2-a]pyrazine (20 g, 95% wt, 82 mmol) in DCM (100 mL) and MeOH (100 mL) was added sodium methoxide (6.8 g, 98% wt, 0.12 mol) at 25° C. The mixture was stirred at 40° C. for 2 h. The reaction mixture was then concentrated under reduced pressure to remove the solvent. It was diluted with water (100 mL) and extracted with EtOAc (100 mL×3). The combined organic layers were washed with saturated aqueous NaCl (100 mL), dried over Na₂SO₄, filtered, and concentrated under reduced pressure to yield the title compound (21.3 g, 84 mmol, 100%, 90% purity) as a white solid. ES/MS m/z: 229.8 (M+H).

### Preparation 326

### 3-Bromoimidazo[1,2-a]pyrazin-8 (7H)-one

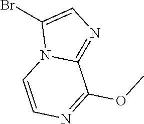

A mixture of 3-bromo-8-methoxyimidazo[1,2-a]pyrazine (21.3 g, 90% wt, 84.1 mmol) and HBr (33% acetic acid) (118 g, 80.0 mL, 33% wt, 481 mmol) was stirred at 80° C. for 16 h. The mixture was then diluted with water (200 mL) and pH was adjusted to around 8 by gradually adding NaOH. The reaction mixture was filtered and the residue was concentrated under reduced pressure to provide the title compound (14.2 g, 60 mmol, 71%, 90% purity) as a white solid. ES/MS m/z: (⁷⁹Br/⁸¹Br) 214.1/216.1 (M+H).

### Preparation 327

### 3-Bromo-7-methylimidazo[1,2-a]pyrazin-8 (7H)-one

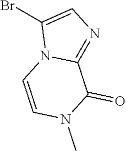

To a mixture of 3-bromoimidazo[1,2-a]pyrazin-8 (7H)-one (14.2 g, 90% wt, 59.7 mmol) and cesium carbonate (48.6 g, 11.9 mL, 149 mmol) in THF (100 mL), was added methyl iodide (25.4 g, 11.6 mL, 179 mmol). The mixture was stirred at 20° C. for 16 h. The mixture was concentrated under reduced pressure to give a crude mixture. The crude mixture was purified by flash silica gel chromatography (Eluent of 0˜6% MeOH/DCM gradient) to provide the title compound (9.6 g, 38 mmol, 63%, 90% Purity) as a white solid. ES/MS m/z: 230.1 (M+H).

### Preparation 328

### Methyl (R)-2-((tert-butoxycarbonyl)amino)-3-(7-methyl-8-oxo-7,8-dihydroimidazo[1,2-a]pyrazin-3-yl)propanoate

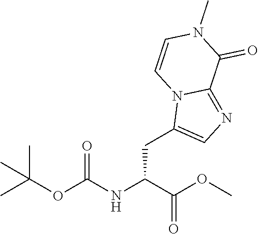 [SMILES: COC(=O)[C@@H](Cc1cnc2c(=O)n(C)ccn12)NC(=O)OC(C)(C)C]

The reaction was carried out in a dried 250 mL three-necked RBF equipped with a magnetic stir bar. A mixture of zinc (2.58 g, 494 μL, 39.5 mmol) and chlorotrimethylsilane (386 mg, 450 μL, 3.55 mmol) in DMA (10 mL) was degassed and purged with nitrogen (N₂) three times. The reaction mixture was stirred at 19° C. for 20 min under a nitrogen atmosphere. Then, methyl(S)-2-((tert-butoxycarbonyl)amino)-3-iodopropanoate (8.70 g, 26.4 mmol) in DMA (15 mL) was added to the mixture. The resulting solution was stirred at 50° C. for 1 h under nitrogen. In a separate 40-mL sealed tube, a mixture of 3-bromo-7-methylimidazo[1,2-a]pyrazin-8 (7H)-one (3.00 g, 90% purity, 11.8 mmol) in DMA (20 mL) was stirred at 80° C. for 30 min with a magnetic stir bar. Next, the mixture of 3-bromo-7-methylimidazo[1,2-a]pyrazin-8 (7H)-one (3.00 g, 90% purity, 11.8 mmol) in DMA (20 mL) and XPhos palladacycle (2.1 g, 2.7 mmol) were added to the 250 mL three-necked RBF, and the reaction mixture was degassed and purged with nitrogen three times. The mixture was then stirred at 80° C. for 2 h under nitrogen. After the reaction was complete, the mixture was cooled to ambient temperature. It was filtered, and the filter cake was washed with EtOAc (30 mL×3). The filtrate was diluted with water (70 mL) and extracted with EtOAc (70 mL×7). The combined organic layers were washed with saturated aqueous NaCl (40 mL×3), dried over anhydrous Na₂SO₄, filtered, and concentrated under reduced pressure to yield a residue. The residue was purified by flash silica gel chromatography (Eluent of 0˜7% MeOH/DCM gradient) to give the title compound (2.21 g, 4.4 mmol, 37%, 70% Purity) as a colorless oil. ES/MS m/z: 351.0 (M+H).

### Preparation 329

### (R)-2-((tert-Butoxycarbonyl)amino)-3-(imidazo[1,2-a]pyrimidin-3-yl)propanoic acid

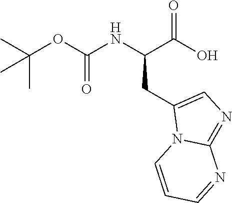 [SMILES: CC(C)(C)OC(=O)N[C@H](Cc1cnc2ncccn12)C(=O)O]

To a solution of methyl (R)-2-((tert-butoxycarbonyl)amino)-3-(imidazo[1,2-a]pyrimidin-3-yl)propanoate (1.04 g, 3.26 mmol) in MeOH (6.0 mL) was added water (6.0 mL) and LiOH (2.0 M in water, 3.3 mL, 6.6 mmol). The reaction mixture was stirred at RT for 3.5 h, then quenched with HCl (4.0 M in 1,4-dioxane) (0.8 mL, 3 mmol). The mixture was concentrated under a stream of N₂ to obtain a quantitative yield of the title compound (1.43 g, 73% purity) as an off-white solid. ES/MS m/z 307.2 (M+H).

The compounds in the following table were prepared in similar manner as described in Preparation 329 using the appropriate methyl ester. Different reaction temperatures and reaction times can be used. Such variances would be apparent to one skilled in the art.

TABLE 36

| Preparation | | | ES-MS |
|---|---|---|---|
| No. | Chemical Name | Structure | m/z |
| 330 | (R)-2-((tert- Butoxycarbonyl)amino)- 3-(6-methylpyridin-3- yl)propanoic acid | 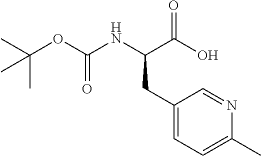 [SMILES: Cc1ccc(C[C@@H](NC(=O)OC(C)(C)C)C(=O)O)cn1] | 281.1 (M + H) |
| 331 | (R)-2-((tert- Butoxycarbonyl)amino)- 3-(isothiazol-4- yl)propanoic acid | 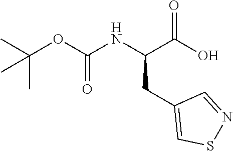 [SMILES: CC(C)(C)OC(=O)N[C@H](Cc1cnsc1)C(=O)O] | 272.9 (M + H) |
| 332 | (R)-2-((tert- Butoxycarbonyl)amino)- 3-(7-methyl-8-oxo-7,8- dihydroimidazo[1,2- a]pyrazin-3-yl)propanoic acid | 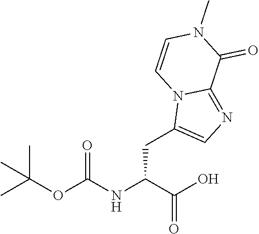 [SMILES: Cn1ccn2c(C[C@@H](NC(=O)OC(C)(C)C)C(=O)O)cnc2c1=O] | 337.2 (M + H) |

### Preparation 333

### tert-Butyl ((2R)-3-(1-(difluoromethyl)-1H-pyrazol-4-yl)-1-((2-methyl-1-(1-(-1-methyl-2-oxopyrrolidin-3-yl)-4-(trifluoromethyl)-1H-imidazol-2-yl)propan-2-yl)amino)-1-oxopropan-2-yl)carbamate (mixture of isomers)

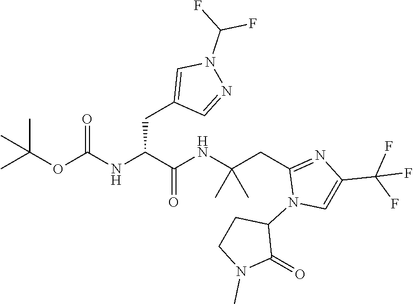 [SMILES: CN1CCC(n2cc(C(F)(F)F)nc2CC(C)(C)NC(=O)[C@@H](Cc2cnn(C(F)F)c2)NC(=O)OC(C)(C)C)C1=O]

To a solution of (R)-2-((tert-butoxycarbonyl)amino)-3-(1-(difluoromethyl)-1H-pyrazol-4-yl)propanoic acid (850 mg, 2.78 mmol), 3-(2-(2-amino-2-methylpropyl)-4-(trifluoromethyl)-1H-imidazol-1-yl)-1-methylpyrrolidin-2-one hydrochloride (racemic mixture) (1.20 g, 80% purity, 2.82 mmol) and HATU (1.61 g, 4.23 mmol) in DMA (10 mL) was added DIEA (2.50 mL, 14.4 mmol). The reaction mixture was stirred at 20° C. for 2 h, then diluted with H₂O (50 mL) and extracted with EtOAc (3×30 mL). The combined organic layers were washed with saturated aqueous NaCl (3×30 mL), dried over anhydrous Na₂SO₄, filtered and concentrated under reduced pressure. The residue was purified on silica, eluting with a gradient of 0-5% MeOH in DCM, to obtain the title compound (1.71 g, 84% purity, 87%) as a yellow solid. ES-MS m/z 592.4 (M+H).

### Preparation 334

### tert-Butyl (R)-(3-(imidazo[1,2-a]pyrimidin-3-yl)-1-((2-methyl-1-(5-(2-methylpyrimidin-5-yl)-3-(trifluoromethyl)-1H-pyrazol-1-yl)propan-2-yl)amino)-1-oxopropan-2-yl)carbamate

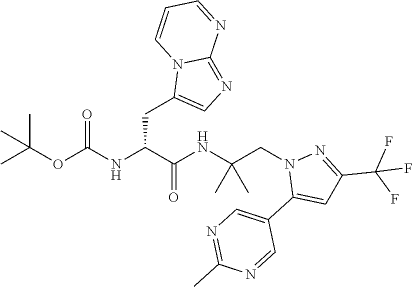 [SMILES: Cc1ncc(-c2cc(C(F)(F)F)nn2CC(C)(C)NC(=O)[C@@H](Cc2cnc3ncccn23)NC(=O)OC(C)(C)C)cn1]

The title compound was prepared essentially as described in Preparation 333 using (R)-2-((tert-butoxycarbonyl)amino)-3-(imidazo[1,2-a]pyrimidin-3-yl)propanoic acid and 2-methyl-1-(5-(2-methylpyrimidin-5-yl)-3-(trifluoromethyl)-1H-pyrazol-1-yl)propan-2-amine hydrochloride in DMF with purification on silica, eluting with a gradient of 0-100% EtOAc in heptane, followed by a gradient of 0-100% (3:1 EtOAc/EtOH) in EtOAc. ES-MS m/z 588.4 (M+H).

### Preparation 335

### tert-Butyl (R)-(1-((1-(5-(1-(difluoromethyl)-2-oxo-1,2-dihydropyridin-4-yl)-3-(trifluoromethyl)-1H-pyrazol-1-yl)-2-methylpropan-2-yl)amino)-3-(6-methylpyridin-3-yl)-1-oxopropan-2-yl)carbamate

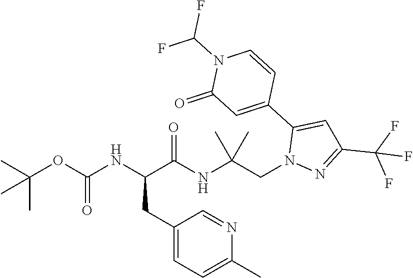 [SMILES: Cc1ccc(C[C@@H](NC(=O)OC(C)(C)C)C(=O)NC(C)(C)Cn2nc(C(F)(F)F)cc2-c2ccn(C(F)F)c(=O)c2)cn1]

The title compound was prepared essentially as described in Preparation 333 using (R)-2-((tert-butoxycarbonyl)amino)-3-(6-methylpyridin-3-yl)propanoic acid and 4-(1-(2-amino-2-methylpropyl)-3-(trifluoromethyl)-1H-pyrazol-5-yl)-1-(difluoromethyl)pyridin-2(1H)-one hydrochloride with purification on silica eluting with 0-80% EtOAc in hexanes. ES-MS m/z 613.4 (M+H).

### Preparation 336

### tert-Butyl (R)-(3-(isothiazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)carbamate

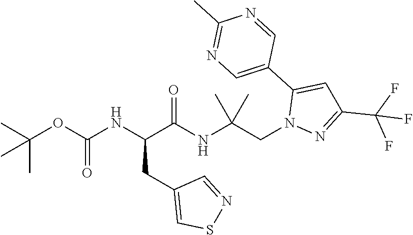 [SMILES: Cc1ncc(-c2cc(C(F)(F)F)nn2CC(C)(C)NC(=O)[C@@H](Cc2cnsc2)NC(=O)OC(C)(C)C)cn1]

The title compound was prepared essentially as described in Preparation 333 using (R)-2-((tert-butoxycarbonyl)amino)-3-(isothiazol-4-yl)propanoic acid and 2-methyl-1-(5-(2-methylpyrimidin-5-yl)-3-(trifluoromethyl)-1H-pyrazol-1-yl)propan-2-amine hydrochloride with purification on silica eluting with 0-50% EtOAc in hexanes. ES-MS m/z 554.2 (M+H).

### Preparation 337

### tert-Butyl (R)-(1-((2-methyl-1-(5-(2-methylpyrimidin-5-yl)-3-(trifluoromethyl)-1H-pyrazol-1-yl)propan-2-yl)amino)-3-(7-methyl-8-oxo-7,8-dihydroimidazo[1,2-a]pyrazin-3-yl)-1-oxopropan-2-yl)carbamate

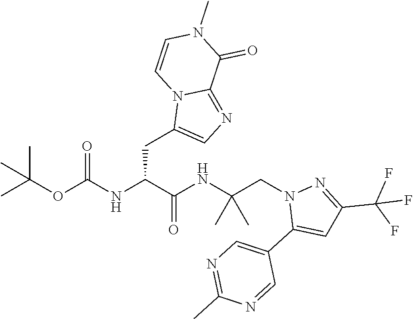 [SMILES: Cc1ncc(-c2cc(C(F)(F)F)nn2CC(C)(C)NC(=O)[C@@H](Cc2cnc3c(=O)n(C)ccn23)NC(=O)OC(C)(C)C)cn1]

The title compound was prepared essentially as described in Preparation 333 using (R)-2-((tert-butoxycarbonyl)amino)-3-(7-methyl-8-oxo-7,8-dihydroimidazo[1,2-a]pyrazin-3-yl)propanoic acid and 2-methyl-1-(5-(2-methylpyrimidin-5-yl)-3-(trifluoromethyl)-1H-pyrazol-1-yl)propan-2-amine hydrochloride with purification on silica eluting with 0-4% MeOH in DCM. ES-MS m/z 618.4 (M+H).

### Preparation 338

### (R)-2-Amino-3-(1-(difluoromethyl)-1H-pyrazol-4-yl)-N-(2-methyl-1-(1-(-1-methyl-2-oxopyrrolidin-3-yl)-4-(trifluoromethyl)-1H-imidazol-2-yl)propan-2-yl)propanamide hydrochloride (Mixture of Isomers)

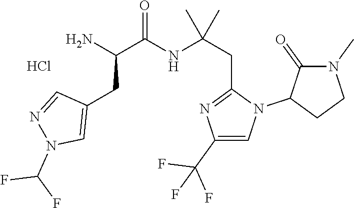 [SMILES: CN1CCC(n2cc(C(F)(F)F)nc2CC(C)(C)NC(=O)[C@H](N)Cc2cnn(C(F)F)c2)C1=O ; Cl]

To a mixture of tert-butyl ((2R)-3-(1-(difluoromethyl)-1H-pyrazol-4-yl)-1-((2-methyl-1-(1-(1-methyl-2-oxopyrrolidin-3-yl)-4-(trifluoromethyl)-1H-imidazol-2-yl)propan-2-yl)amino)-1-oxopropan-2-yl)carbamate (mixture of isomers) (1.71 g, 84% purity, 2.43 mmol) in MeOH (10 mL) was added 2.0 M HCl in dioxane (20.0 mL, 40.0 mmol). The reaction mixture was stirred at 20° C. for 1 h, then concentrated under reduced pressure. The crude residue was triturated with DCM (15 mL) at 20° C. for 0.5 h. The mixture was filtered, and the filter cake was vacuum dried to give the title compound (1.29 g, 97%) as an off-white solid. ES-MS m/z 492.0 (M+H).

The compounds in the following table were prepared in similar manner as described in Preparation 338 using the appropriate carbamate and without DCM trituration or any further purification. Different equivalents of HCl (5-21 eq) and reaction times (1-20 h) can be used. Such variances would be apparent to one skilled in the art.

TABLE 37

| Preparation | | | ES-MS |
|---|---|---|---|
| No. | Chemical Name | Structure | m/z |
| 339 | (R)-2-Amino-N-(2- methyl-1-(5-(2- methylpyrimidin-5-yl)- 3-(trifluoromethyl)-1H- pyrazol-1-yl)propan-2- yl)-3-(1-(methyl-d3)- 1H-pyrazol-4- yl)propanamide hydrochloride | 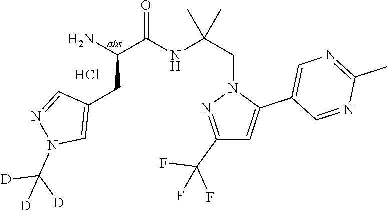 [SMILES: [2H]C([2H])([2H])n1cc(C[C@@H](N)C(=O)NC(C)(C)Cn2nc(C(F)(F)F)cc2-c2cnc(C)nc2)cn1 ; Cl] | 454.3 (M + H) |
| 340 | (R)-2-Amino-3- (imidazo[1,2- a]pyrimidin-3-yl)-N-(2- methyl-1-(5-(2- methylpyrimidin-5-yl)- 3-(trifluoromethyl)-1H- pyrazol-1-yl)propan-2- yl)propanamide hydrochloride | 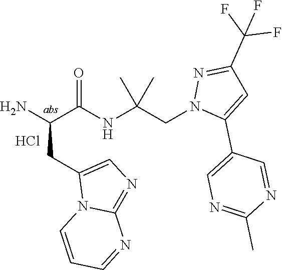 [SMILES: Cl ; Cc1ncc(-c2cc(C(F)(F)F)nn2CC(C)(C)NC(=O)[C@H](N)Cc2cnc3ncccn23)cn1] | 488.3 (M + H) |
| 341 | (R)-2-Amino-N-(1-(5- (1-(difluoromethyl)-2- oxo-1,2-dihydropyridin- 4-yl)-3- (trifluoromethyl)-1H- pyrazol-1-yl)-2- methylpropan-2-yl)-3- (6-methylpyridin-3- yl)propanamide hydrochloride | 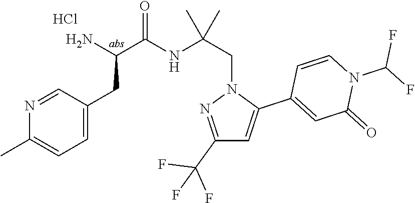 [SMILES: Cc1ccc(C[C@@H](N)C(=O)NC(C)(C)Cn2nc(C(F)(F)F)cc2-c2ccn(C(F)F)c(=O)c2)cn1 ; Cl] | 513.3 (M + H) |
| 342 | (R)-2-Amino-3- (isothiazol-4-yl)-N-(2- methyl-1-(5-(2- methylpyrimidin-5-yl)- 3-(trifluoromethyl)-1H- pyrazol-1-yl)propan-2- yl)propanamide hydrochloride | 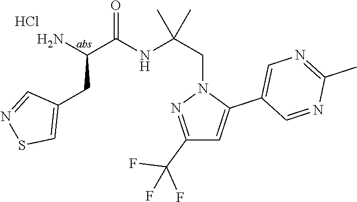 [SMILES: Cc1ncc(-c2cc(C(F)(F)F)nn2CC(C)(C)NC(=O)[C@H](N)Cc2cnsc2)cn1 ; Cl] | 454.1 (M + H) |
| 343 | (R)-2-Amino-N-(2- methyl-1-(5-(2- methylpyrimidin-5-yl)- 3-(trifluoromethyl)-1H- pyrazol-1-yl)propan-2- yl)-3-(7-methyl-8-oxo- 7,8-dihydroimidazo[1,2- a]pyrazin-3- yl)propanamide hydrochloride | 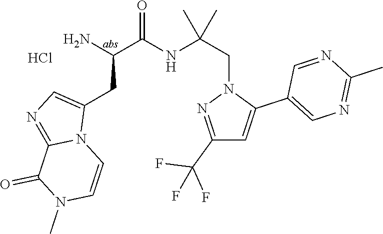 [SMILES: Cc1ncc(-c2cc(C(F)(F)F)nn2CC(C)(C)NC(=O)[C@H](N)Cc2cnc3c(=O)n(C)ccn23)cn1 ; Cl] | 518.2 (M + H) |

### Preparation 344

### Methyl (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoate

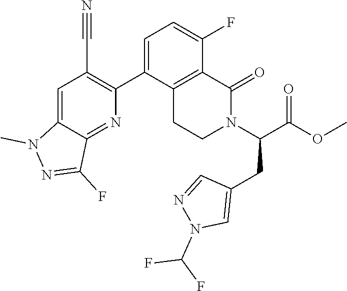

A mixture of 5-chloro-3-fluoro-1-methyl-1H-pyrazolo[4,3-b]pyridine-6-carbonitrile (3.68 g, 98% purity, 17.1 mmol), (R)-(2-(3-(1-(difluoromethyl)-1H-pyrazol-4-yl)-1-methoxy-1-oxopropan-2-yl)-8-fluoro-1-oxo-1,2,3,4-tetrahydroisoquinolin-5-yl)boronic acid (8.28 g, 85% purity, 17.1 mmol), sodium carbonate (5.9 g, 56 mmol) and XPhos Pd G3 (3.1 g, 3.7 mmol) in 1,4-dioxane (150 mL) and water (15 mL) was degassed and purged with N₂ (3×), then stirred at 90° C. for 1 h under N₂. The reaction mixture was filtered, and the filtrate was concentrated under reduced pressure, then purified on silica (eluting with a gradient of 0-5% MeOH in DCM) provided a yellow solid (10.3 g, 82% purity) which was dissolved in DCM (20 mL). The solution was added dropwise to vigorously stirring hexanes (50 mL), and stirring was continued for 10 min, then stopped. After standing for 30 min, the solids were collected by filtration. Purification on silica (eluting with a gradient of 0-3% (0.5% formic acid/MeOH) in DCM), provided the title compound (6.27 g, 72%) as a light-yellow solid. ES-MS m/z 542.2 (M+H).

The compounds in the following table were prepared in similar manner as described in Preparation 344 using (R)-(2-(3-(1-(difluoromethyl)-1H-pyrazol-4-yl)-1-methoxy-1-oxopropan-2-yl)-8-fluoro-1-oxo-1,2,3,4-tetrahydroisoquinolin-5-yl)boronic acid or (R)-(8-fluoro-2-(1-methoxy-3-(1-methyl-1H-pyrazol-4-yl)-1-oxopropan-2-yl)-1-oxo-1,2,3,4-tetrahydroisoquinolin-5-yl)boronic acid and the appropriate heteroaryl halide. Different reaction temperatures, reaction times, catalysts and purification conditions can be used. Such variances would be apparent to one skilled in the art.

TABLE 38

| Preparation | | | ES-MS |
|---|---|---|---|
| No. | Chemical Name | Structure | m/z |
| 345^{a,b} | Methyl (R)-2-(5-(3- chloro-6-cyano-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)propanoate | 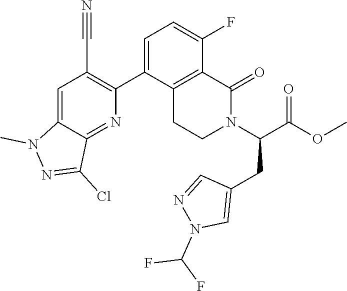 | 558.0 (M + H) |
| 346^{c} | Methyl (R)-2-(5-(3,6- difluoro-1-(methyl-d3)- 1H-pyrazolo[4,3- b]pyridin-5-yl)-8-fluoro- 1-oxo-3,4- dihydroisoquinolin- 2(1H)-yl)-3-(1- (difluoromethyl)-1H- pyrazol-4-yl)propanoate | 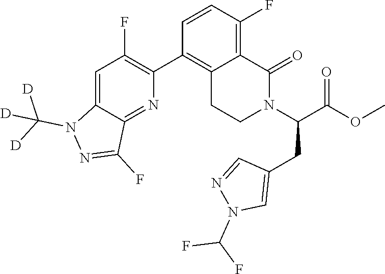 [SMILES: [2H]C([2H])([2H])n1nc(F)c2nc(-c3ccc(F)c4c3CCN([C@H](Cc3cnn(C(F)F)c3)C(=O)OC)C4=O)c(F)cc21] | 538.4 (M + H) |
| 347^{a,d} | Methyl (R)-2-(5-(3- chloro-6-fluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)propanoate | 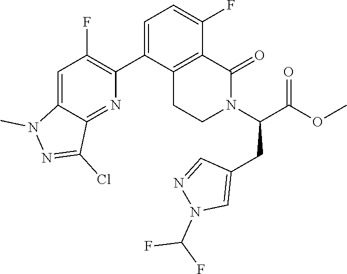 | 551.2 (M + H) |
| 348^{e} | Methyl (R)-3-(1- (difluoromethyl)-1H- pyrazol-4-yl)-2-(8- fluoro-5-(4-methoxy-1- methyl-6-oxo-1,6- dihydropyridazin-3-yl)- 1-oxo-3,4- dihydroisoquinolin- 2(1H)-yl)propanoate | 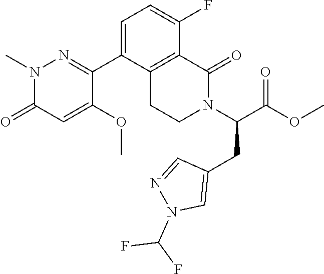 [SMILES: COC(=O)[C@@H](Cc1cnn(C(F)F)c1)N1CCc2c(-c3nn(C)c(=O)cc3OC)ccc(F)c2C1=O] | 506.2 (M + H) |
| 349^{f} | Methyl (R)-3-(1- (difluoromethyl)-1H- pyrazol-4-yl)-2-(8- fluoro-5-(6-fluoro-1- methyl-1H- pyrazolo[4,3-b]pyridin- 5-yl)-1-oxo-3,4- dihydroisoquinolin- 2(1H)-yl)propanoate | 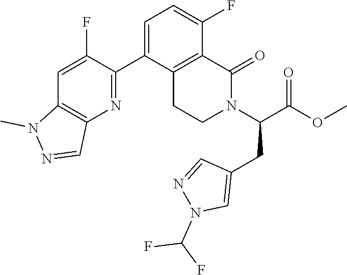 | 517.3 (M + H) |
| 350^{a,g} | Methyl (R)-3-(1- (difluoromethyl)-1H- pyrazol-4-yl)-2-(8- fluoro-5-(5-fluoro-2- methoxypyrimidin-4-yl)- 1-oxo-3,4- dihydroisoquinolin- 2(1H)-yl)propanoate | 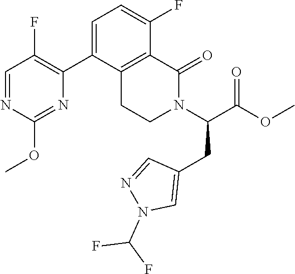 [SMILES: COC(=O)[C@@H](Cc1cnn(C(F)F)c1)N1CCc2c(-c3nc(OC)ncc3F)ccc(F)c2C1=O] | 494.1 (M + H) |
| 351^{h} | Methyl (R)-2-(5-(5- cyano-3-fluoro-6- methylpyridin-2-yl)-8- fluoro-1-oxo-3,4- dihydroisoquinolin- 2(1H)-yl)-3-(1- (difluoromethyl)-1H- pyrazol-4-yl)propanoate | 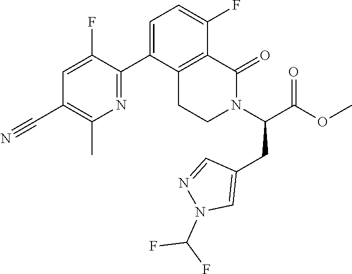 [SMILES: COC(=O)[C@@H](Cc1cnn(C(F)F)c1)N1CCc2c(-c3nc(C)c(C#N)cc3F)ccc(F)c2C1=O] | 502.1 (M + H) |
| 352^{a,i} | Methyl (R)-2-(5-(3- chloro-6-cyano-1- methyl-1H- pyrazolo[4,3-b]pyridin- 5-yl)-8-fluoro-1-oxo- 3,4-dihydroisoquinolin- 2(1H)-yl)-3-(1-methyl- 1H-pyrazol-4- yl)propanoate | 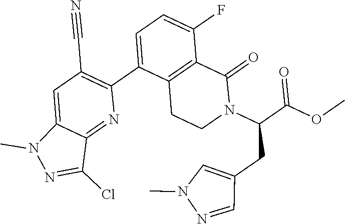 | 522.0 (M + H) |

^{a}Using 0.1 eq. of 1,1′-bis(di-t-butylphosphino)ferrocene palladium dichloride as catalyst
^{b}From 3,5-dichloro-1-methyl-1H-pyrazolo[4,3-b]pyridine-6-carbonitrile; purified on silica (eluting with a gradient of 0-60% EtOAc in hexanes)
^{c}From 5-bromo-3,6-difluoro-1-(methyl-d3)-1H-pyrazolo[4,3-b]pyridine (eluting with a gradient of 0-2% MeOH in DCM)
^{d}80° C., 3h, From 5-bromo-3-chloro-6-fluoro-1-methyl-1H-pyrazolo[4,3-b]pyridine; purified on silica (eluting with a gradient of 0-50% EtOAc in hexanes)
^{e}90° C., 80 min, from 6-chloro-5-methoxy-2-methylpyridazin-3(2H)-one and using 0.15 eq. of XPhos Pd G4 as catalyst; purified on silica (eluting with a gradient of 0-8% MeOH in DCM)
^{f}From 5-bromo-6-fluoro-1-methyl-1H-pyrazolo[4,3-b]pyridine; purified on silica (eluting with a gradient of 0-2% MeOH in DCM)
^{g}From 4-chloro-5-fluoro-2-methoxypyrimidine; purified on silica (eluting with a gradient of 0-60% EtOAc in hexanes)
^{h}From 6-chloro-5-fluoro-2-methylnicotinonitrile; purified on silica (eluting with a gradient of 0-44% EtOAc in hexanes)
ⁱFrom 3,5-dichloro-1-methyl-1H-pyrazolo[4,3-b]pyridine-6-carbonitrile; purified on silica (eluting with isocratic 2% MeOH in DCM)

### Preparation 353

### (R)-2-(5-(6-Cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid

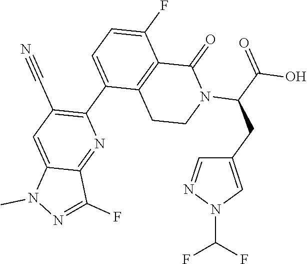 [SMILES: Cn1nc(F)c2nc(-c3ccc(F)c4c3CCN([C@H](Cc3cnn(C(F)F)c3)C(=O)O)C4=O)c(C#N)cc21]

To a mixture of methyl (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoate (2.34 g, 86% purity, 3.72 mmol) in tBuOH (60 mL) and water (26 mL) at 0° C. was added 1N aq. LiOH (10.5 mL, 10.5 mmol). The reaction mixture was stirred at 0° C. for 3 h. The mixture was adjusted to approximately pH 3 with 1 N aq. HCl, then diluted with H₂O (80 mL) and extracted with EtOAc (2×100 mL). The combined organic layers were dried over anhydrous Na₂SO₄, filtered and concentrated under reduced pressure. The residue was purified by reversed-phase preparative HPLC (column: PrePulite XP tC18, 50×250 mm, 10 μm; eluting with a gradient of 15-55% ACN in 0.225% aq. formic acid) to obtain the title compound (1.22 g, 59%) as a light yellow solid. ES-MS m/z 528.0 (M+H).

The compounds in the following table were prepared in similar manner as described in Preparation 353 using the appropriate methyl ester prepared above. The compounds may be left as a crude residue or further purified, which would be apparent to one skilled in the art.

TABLE 39

| Preparation | | | ES-MS |
|---|---|---|---|
| No | Chemical Name | Structure | m/z |
| 354 | (R)-2-(5-(3-Chloro-6- cyano-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)propanoic acid | 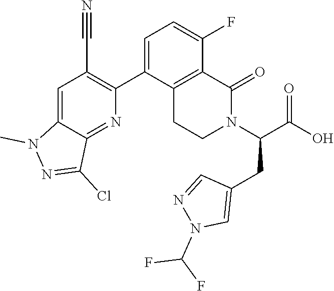 [SMILES: Cn1nc(Cl)c2nc(-c3ccc(F)c4c3CCN([C@H](Cc3cnn(C(F)F)c3)C(=O)O)C4=O)c(C#N)cc21] | 544.1 (M + H) |
| 355 | (R)-2-(5-(3,6-Difluoro- 1-(methyl-d3)-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)propanoic acid | 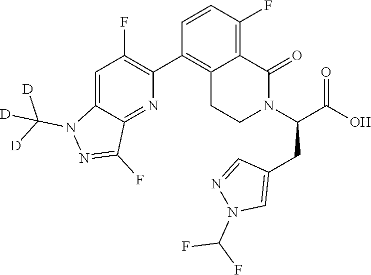 [SMILES: [2H]C([2H])([2H])n1nc(F)c2nc(-c3ccc(F)c4c3CCN([C@H](Cc3cnn(C(F)F)c3)C(=O)O)C4=O)c(F)cc21] | 524.3 (M + H) |
| 356 | (R)-2-(5-(3-Chloro-6- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)propanoic acid | 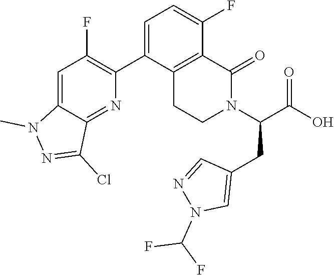 [SMILES: Cn1nc(Cl)c2nc(-c3ccc(F)c4c3CCN([C@H](Cc3cnn(C(F)F)c3)C(=O)O)C4=O)c(F)cc21] | 537.1 (M + H) |
| 357 | (R)-3-(1- (Difluoromethyl)-1H- pyrazol-4-yl)-2-(8- fluoro-5-(4-methoxy-1- methyl-6-oxo-1,6- dihydropyridazin-3-yl)- 1-oxo-3,4- dihydroisoquinolin- 2(1H)-yl)propanoic acid | 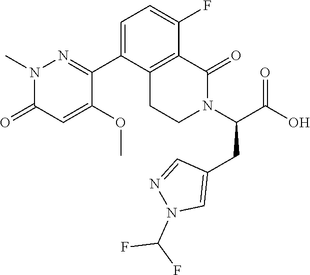 [SMILES: COc1cc(=O)n(C)nc1-c1ccc(F)c2c1CCN([C@H](Cc1cnn(C(F)F)c1)C(=O)O)C2=O] | 492.0 (M + H) |
| 358 | (R)-3-(1- (Difluoromethyl)-1H- pyrazol-4-yl)-2-(8- fluoro-5-(6-fluoro-1- methyl-1H- pyrazolo[4,3-b]pyridin- 5-yl)-1-oxo-3,4- dihydroisoquinolin- 2(1H)-yl)propanoic acid | 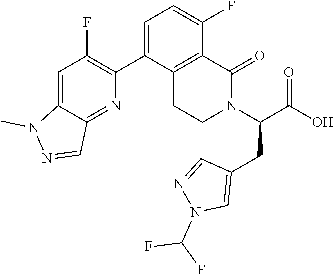 [SMILES: Cn1ncc2nc(-c3ccc(F)c4c3CCN([C@H](Cc3cnn(C(F)F)c3)C(=O)O)C4=O)c(F)cc21] | 503.0 (M + H) |
| 359 | (R)-3-(1- (Difluoromethyl)-1H- pyrazol-4-yl)-2-(8- fluoro-5-(5-fluoro-2- methoxypyrimidin-4-yl)- 1-oxo-3,4- dihydroisoquinolin- 2(1H)-yl)propanoic acid | 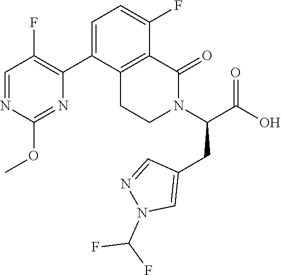 [SMILES: COc1ncc(F)c(-c2ccc(F)c3c2CCN([C@H](Cc2cnn(C(F)F)c2)C(=O)O)C3=O)n1] | 480.1 (M + H) |
| 360 | (R)-2-(5-(5-Cyano-3- fluoro-6-methylpyridin- 2-yl)-8-fluoro-1-oxo- 3,4-dihydroisoquinolin- 2(1H)-yl)-3-(1- (difluoromethyl)-1H- pyrazol-4-yl)propanoic acid | 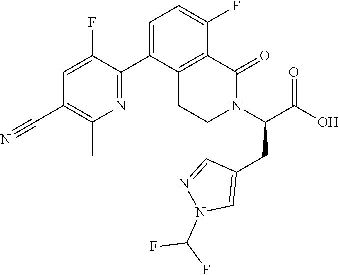 [SMILES: Cc1nc(-c2ccc(F)c3c2CCN([C@H](Cc2cnn(C(F)F)c2)C(=O)O)C3=O)c(F)cc1C#N] | 488.1 (M + H) |
| 361 | (R)-2-(5-(3-Chloro-6- cyano-1-methyl-1H- pyrazolo[4,3-b]pyridin- 5-yl)-8-fluoro-1-oxo- 3,4-dihydroisoquinolin- 2(1H)-yl)-3-(1-methyl- 1H-pyrazol-4- yl)propanoic acid | 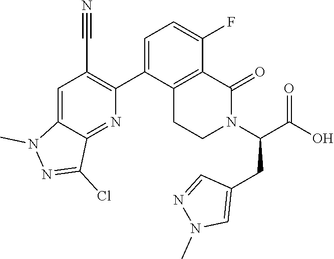 [SMILES: Cn1cc(C[C@H](C(=O)O)N2CCc3c(-c4nc5c(Cl)nn(C)c5cc4C#N)ccc(F)c3C2=O)cn1] | 508.1 (M + H) |

### Preparation 362

### 3-Bromo-2-(2-chloroethyl)-N-((2R)-3-(1-(difluoromethyl)-1H-pyrazol-4-yl)-1-((2-methyl-1-(1-(-1-methyl-2-oxopyrrolidin-3-yl)-4-(trifluoromethyl)-1H-imidazol-2-yl)propan-2-yl)amino)-1-oxopropan-2-yl)-6-fluorobenzamide (mixture of isomers)

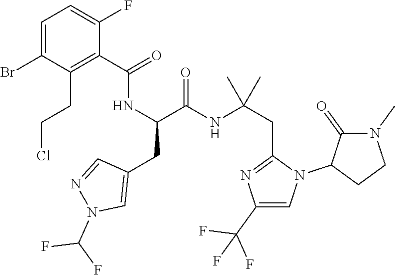 [SMILES: CN1CCC(n2cc(C(F)(F)F)nc2CC(C)(C)NC(=O)[C@@H](Cc2cnn(C(F)F)c2)NC(=O)c2c(F)ccc(Br)c2CCCl)C1=O]

A mixture of (R)-2-amino-3-(1-(difluoromethyl)-1H-pyrazol-4-yl)-N-(2-methyl-1-(1-(-1-methyl-2-oxopyrrolidin-3-yl)-4-(trifluoromethyl)-1H-imidazol-2-yl)propan-2-yl)propanamide hydrochloride (mixture of isomers) (794 mg, 96% purity, 1.44 mmol) and TEA (2.00 mL, 14.3 mmol) in THF (10 mL) was cooled to 0° C. in an ice-water bath then a solution of 3-bromo-2-(2-chloroethyl)-6-fluorobenzoyl chloride (938 mg, 74% purity, 2.31 mmol) in THF (10 mL) was added dropwise. The reaction mixture was stirred at 20° C. for 1 h, then diluted with H₂O (20 mL) and extracted with EtOAc (3×20 mL). The combined organic layers were washed with saturated aqueous NaCl (20 mL), dried over anhydrous Na₂SO₄, filtered, and concentrated under reduced pressure to give a residue. Purification on silica, eluting with a gradient of 0-5% MeOH in DCM, provided the title compound (1.03 g, 83%) as a yellow solid. ES-MS m/z (⁷⁹Br/⁸¹Br, ³⁵Cl/³⁷Cl) 754.2, 755.9, 758.1 (M+H).

The compounds in the following table were prepared in similar manner as described in Preparation 362 using the appropriate primary amine and acid chloride prepared above. Different reaction temperatures and reaction times can be used. Such variances would be apparent to one skilled in the art.

TABLE 40

| Preparation | | | ES-MS |
|---|---|---|---|
| No. | Chemical Name | Structure | m/z |
| 363^{a} | (R)-3-Bromo-2-(2- chloroethyl)-6-fluoro-N- (1-((2-methyl-1-(5-(2- methylpyrimidin-5-yl)- 3-(trifluoromethyl)-1H- pyrazol-1-yl)propan-2- yl)amino)-3-(1-(methyl- d3)-1H-pyrazol-4-yl)-1- oxopropan-2- yl)benzamide | 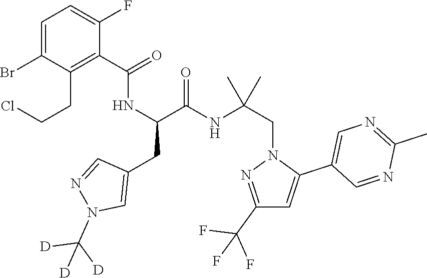 [SMILES: [2H]C([2H])([2H])n1cc(C[C@@H](NC(=O)c2c(F)ccc(Br)c2CCCl)C(=O)NC(C)(C)Cn2nc(C(F)(F)F)cc2-c2cnc(C)nc2)cn1] | 718.4 (M + H) |
| 364 | (R)-3-Bromo-2-(2- chloroethyl)-N-(1-((1- (5-(1-(difluoromethyl)- 2-oxo-1,2- dihydropyridin-4-yl)-3- (trifluoromethyl)-1H- pyrazol-1-yl)-2- methylpropan-2- yl)amino)-3-(6- methylpyridin-3-yl)-1- oxopropan-2-yl)-6- fluorobenzamide | 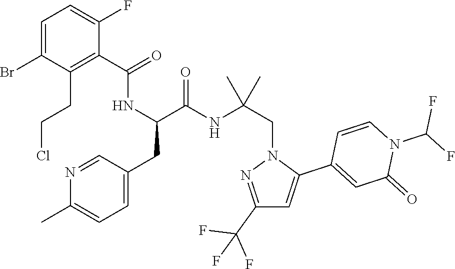 [SMILES: Cc1ccc(C[C@@H](NC(=O)c2c(F)ccc(Br)c2CCCl)C(=O)NC(C)(C)Cn2nc(C(F)(F)F)cc2-c2ccn(C(F)F)c(=O)c2)cn1] | 777.1 (M + H) |
| 365^{a,b} | (R)-3-Bromo-2-(2- chloroethyl)-6-fluoro-N- (3-(imidazo[1,2- a]pyrimidin-3-yl)-1-((2- methyl-1-(5-(2- methylpyrimidin-5-yl)- 3-(trifluoromethyl)-1H- pyrazol-1-yl)propan-2- yl)amino)-1-oxopropan- 2-yl)benzamide | 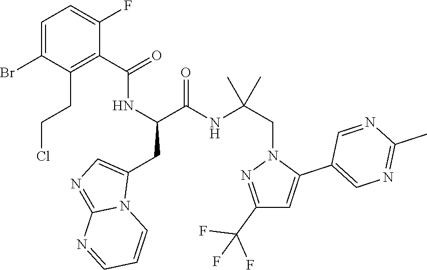 [SMILES: Cc1ncc(-c2cc(C(F)(F)F)nn2CC(C)(C)NC(=O)[C@@H](Cc2cnc3ncccn23)NC(=O)c2c(F)ccc(Br)c2CCCl)cn1] | 752.2 (M + H) |
| 366 | (R)-3-Bromo-2-(2- chloroethyl)-6-fluoro-N- (1-((2-methyl-1-(5-(2- methylpyrimidin-5-yl)- 3-(trifluoromethyl)-1H- pyrazol-1-yl)propan-2- yl)amino)-3-(7-methyl- 8-oxo-7,8- dihydroimidazo[1,2- a]pyrazin-3-yl)-1- oxopropan-2- yl)benzamide | 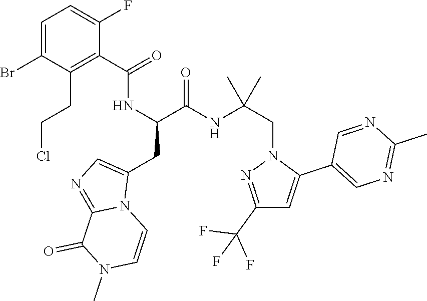 [SMILES: Cc1ncc(-c2cc(C(F)(F)F)nn2CC(C)(C)NC(=O)[C@@H](Cc2cnc3c(=O)n(C)ccn23)NC(=O)c2c(F)ccc(Br)c2CCCl)cn1] | 782.1 (M + H) |
| 367 | (R)-3-Bromo-2-(2- chloroethyl)-6-fluoro-N- (3-(isothiazol-4-yl)-1- ((2-methyl-1-(5-(2- methylpyrimidin-5-yl)- 3-(trifluoromethyl)-1H- pyrazol-1-yl)propan-2- yl)amino)-1-oxopropan- 2-yl)benzamide | 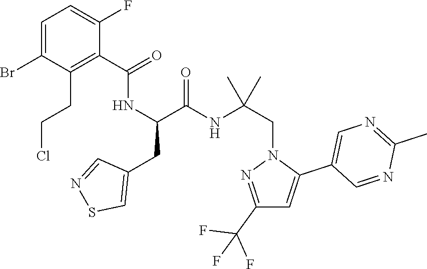 [SMILES: Cc1ncc(-c2cc(C(F)(F)F)nn2CC(C)(C)NC(=O)[C@@H](Cc2cnsc2)NC(=O)c2c(F)ccc(Br)c2CCCl)cn1] | 717.8 (M + H) |

^{a}The reaction solvent used was DCM
^{b}The reaction base was DIEA

### Preparation 368

### (2R)-2-(5-Bromo-8-fluoro-1-oxo-3,4-dihydroisoquinolin-2(1H)-yl)-3-(1-(difluoromethyl)-1H-pyrazol-4-yl)-N-(2-methyl-1-(1-(-1-methyl-2-oxopyrrolidin-3-yl)-4-(trifluoromethyl)-1H-imidazol-2-yl)propan-2-yl)propanamide (mixture of isomers)

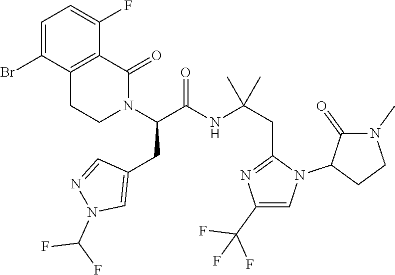 [SMILES: CN1CCC(n2cc(C(F)(F)F)nc2CC(C)(C)NC(=O)[C@@H](Cc2cnn(C(F)F)c2)N2CCc3c(Br)ccc(F)c3C2=O)C1=O]

In a dried 100 mL RBF containing a magnetic stir bar, a mixture of 3-bromo-2-(2-chloroethyl)-N-((2R)-3-(1-(difluoromethyl)-1H-pyrazol-4-yl)-1-((2-methyl-1-(1-((RS)-1-methyl-2-oxopyrrolidin-3-yl)-4-(trifluoromethyl)-1H-imidazol-2-yl)propan-2-yl)amino)-1-oxopropan-2-yl)-6-fluorobenzamide (1.03 g, 88% purity, 1.20 mmol) in ACN (10 mL) was treated with Cs₂CO₃ (595 mg, 1.83 mmol) and stirred at 20° C. for 2 h. The reaction mixture was diluted with H₂O (20 mL) and extracted with EtOAc (20 mL×3). The combined organics were dried over Na₂SO₄, filtered, and concentrated under reduced pressure. Column chromatography (SiO₂, gradient 0-5% MeOH in DCM) provided the title compound (831 mg, 88%) as a white solid. ES-MS m/z 717.9/720.0 (M+H) (Br^{79/81}).

The compounds in the following table were prepared in similar manner as described in Preparation 368 using the appropriate alkyl halide prepared above. Base such as cesium carbonate can be used in solvent such as ACN, DMF and NMP. Different reaction temperatures and reaction times can be used. Such variances would be apparent to one skilled in the art.

TABLE 41

| Preparation | | | ES-MS |
|---|---|---|---|
| No. | Chemical Name | Structure | m/z |
| 369 | (R)-2-(5-bromo-8- fluoro-1-oxo-3,4- dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)-3-(1-(methyl-d3)- 1H-pyrazol-4- yl)propanamide | 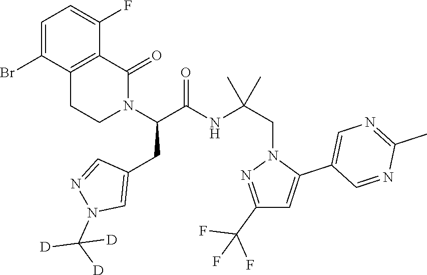 [SMILES: [2H]C([2H])([2H])n1cc(C[C@H](C(=O)NC(C)(C)Cn2nc(C(F)(F)F)cc2-c2cnc(C)nc2)N2CCc3c(Br)ccc(F)c3C2=O)cn1] | 682.0 (M + H) |
| 370 | (R)-2-(5-Bromo-8- fluoro-1-oxo-3,4- dihydroisoquinolin- 2(1H)-yl)-N-(1-(5-(1- (difluoromethyl)-2-oxo- 1,2-dihydropyridin-4- yl)-3-(trifluoromethyl)- 1H-pyrazol-1-yl)-2- methylpropan-2-yl)-3- (6-methylpyridin-3- yl)propanamide | 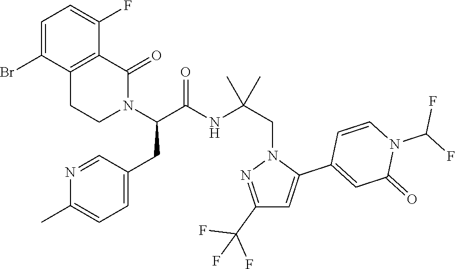 [SMILES: Cc1ccc(C[C@H](C(=O)NC(C)(C)Cn2nc(C(F)(F)F)cc2-c2ccn(C(F)F)c(=O)c2)N2CCc3c(Br)ccc(F)c3C2=O)cn1] | (⁷⁹Br/ ⁸¹Br) 739.3/ 741.3 (M + H) |
| 371 | (R)-2-(5-Bromo-8- fluoro-1-oxo-3,4- dihydroisoquinolin- 2(1H)-yl)-3- (imidazo[1,2- a]pyrimidin-3-yl)-N-(2- methyl-1-(5-(2- methylpyrimidin-5-yl)- 3-(trifluoromethyl)-1H- pyrazol-1-yl)propan-2- yl)propanamide | 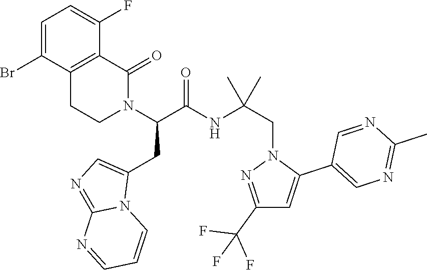 [SMILES: Cc1ncc(-c2cc(C(F)(F)F)nn2CC(C)(C)NC(=O)[C@@H](Cc2cnc3ncccn23)N2CCc3c(Br)ccc(F)c3C2=O)cn1] | (⁷⁹Br/ ⁸¹Br) 714.2/ 716.2 (M + H) |
| 372 | (R)-2-(5-bromo-8- fluoro-1-oxo-3,4- dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)-3-(7-methyl-8-oxo- 7,8-dihydroimidazo[1,2- a]pyrazin-3- yl)propanamide | 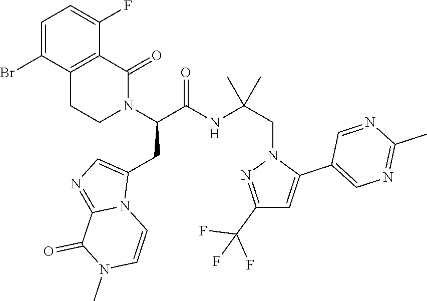 [SMILES: Cc1ncc(-c2cc(C(F)(F)F)nn2CC(C)(C)NC(=O)[C@@H](Cc2cnc3c(=O)n(C)ccn23)N2CCc3c(Br)ccc(F)c3C2=O)cn1] | (⁷⁹Br/ ⁸¹Br) 744.2/ 746.2 (M + H) |
| 373 | (R)-2-(5-bromo-8- fluoro-1-oxo-3,4- dihydroisoquinolin- 2(1H)-yl)-3-(isothiazol- 4-yl)-N-(2-methyl-1-(5- (2-methylpyrimidin-5- yl)-3-(trifluoromethyl)- 1H-pyrazol-1-yl)propan- 2-yl)propanamide | 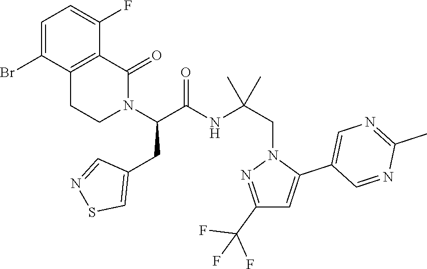 [SMILES: Cc1ncc(-c2cc(C(F)(F)F)nn2CC(C)(C)NC(=O)[C@@H](Cc2cnsc2)N2CCc3c(Br)ccc(F)c3C2=O)cn1] | (⁷⁹Br/ ⁸¹Br) 679.9/ 681.9 (M + H) |

### Preparation 374

### (2R)-3-(1-(Difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(1-(-1-methyl-2-oxopyrrolidin-3-yl)-4-(trifluoromethyl)-1H-imidazol-2-yl)propan-2-yl)propanamide (Mixture of Isomers)

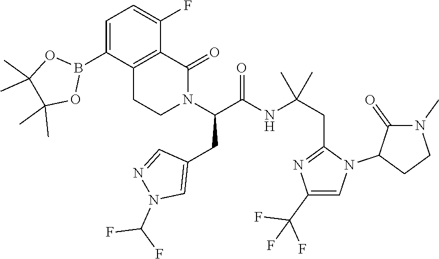

In a N₂-filled glovebox, (2R)-2-(5-bromo-8-fluoro-1-oxo-3,4-dihydroisoquinolin-2(1H)-yl)-3-(1-(difluoromethyl)-1H-pyrazol-4-yl)-N-(2-methyl-1-(1-((RS)-1-methyl-2-oxopyrrolidin-3-yl)-4-(trifluoromethyl)-1H-imidazol-2-yl)propan-2-yl)propanamide (830 mg, 91% purity, 1.05 mmol), bis(pinacolato)diborane (2.142 g, 8.435 mmol), potassium acetate (327 mg, 3.33 mmol) and (2-dicyclohexylphosphino-2′,6′-dimethoxybiphenyl)[2-(2′-amino-1,1′-biphenyl)]palladium(II) methanesulfonate (92.4 mg, 106 μmol) were added to a dried 40 mL vial. DMF (15 mL) was added, and the sealed vial was stirred at 100° C. for 16 h, diluted with H₂O (50 mL) and extracted with EtOAc (30 mL×3). The combined organics were washed with saturated aqueous NaCl (30 mL×3), dried over Na₂SO₄, filtered, and concentrated under reduced pressure. Column chromatography (SiO₂, gradient 0-4% MeOH in DCM) provided the title compound (624 mg, 60%) as a white solid. ES-MS m/z 766.2 (M+H).

The compounds in the following table were prepared in similar manner as described in Preparation 374 using the appropriate aryl halide prepared above. Different reaction temperatures, reaction times and catalysts can be used. Such variances would be apparent to one skilled in the art.

TABLE 42

| Preparation | | | ES-MS |
|---|---|---|---|
| No. | Chemical Name | Structure | m/z |
| 375^{a} | (R)-2-(8-fluoro-1-oxo-5- (4,4,5,5-tetramethyl- 1,3,2-dioxaborolan-2- yl)-3,4- dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)-3-(1-(methyl-d3)- 1H-pyrazol-4- yl)propanamide | 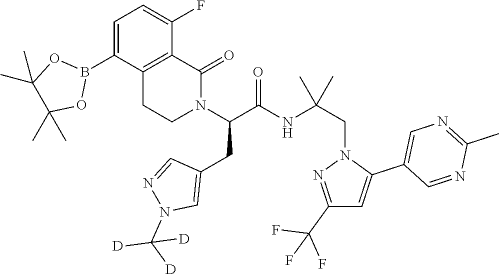 | 728.6 (M + H) |
| 376^{b} | (R)-N-(1-(5-(1- (Difluoromethyl)-2-oxo- 1,2-dihydropyridin-4- yl)-3-(trifluoromethyl)- 1H-pyrazol-1-yl)-2- methylpropan-2-yl)-2- (8-fluoro-1-oxo-5- (4,4,5,5-tetramethyl- 1,3,2-dioxaborolan-2- yl)-3,4- dihydroisoquinolin- 2(1H)-yl)-3-(6- methylpyridin-3- yl)propanamide | 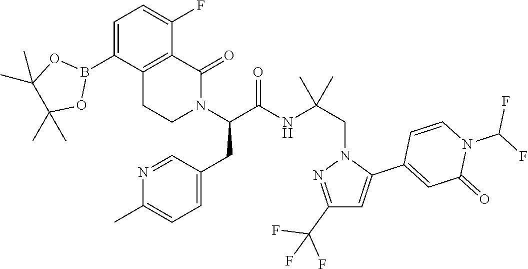 [SMILES: Cc1ccc(C[C@H](C(=O)NC(C)(C)Cn2nc(C(F)(F)F)cc2-c2ccn(C(F)F)c(=O)c2)N2CCc3c(B4OC(C)(C)C(C)(C)O4)ccc(F)c3C2=O)cn1] | 787.5 (M + H) |
| 377^{a} | (R)-2-(8-Fluoro-1-oxo- 5-(4,4,5,5-tetramethyl- 1,3,2-dioxaborolan-2- yl)-3,4- dihydroisoquinolin- 2(1H)-yl)-3- (imidazo[1,2- a]pyrimidin-3-yl)-N-(2- methyl-1-(5-(2- methylpyrimidin-5-yl)- 3-(trifluoromethyl)-1H- pyrazol-1-yl)propan-2- yl)propanamide | 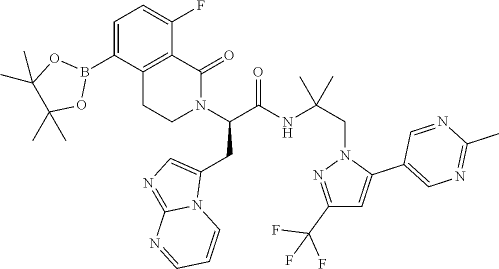 | 680.4 (M + H) |
| 378^{b} | (R)-2-(8-Fluoro-1-oxo- 5-(4,4,5,5-tetramethyl- 1,3,2-dioxaborolan-2- yl)-3,4- dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)-3-(7-methyl-8-oxo- 7,8-dihydroimidazo[1,2- a]pyrazin-3- yl)propanamide | 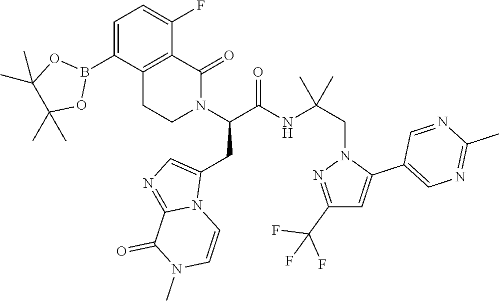 | 792.2 (M + H) |
| 379^{b} | (R)-2-(8-fluoro-1-oxo-5- (4,4,5,5-tetramethyl- 1,3,2-dioxaborolan-2- yl)-3,4- dihydroisoquinolin- 2(1H)-yl)-3-(isothiazol- 4-yl)-N-(2-methyl-1-(5- (2-methylpyrimidin-5- yl)-3-(trifluoromethyl)- 1H-pyrazol-1-yl)propan- 2-yl)propanamide | 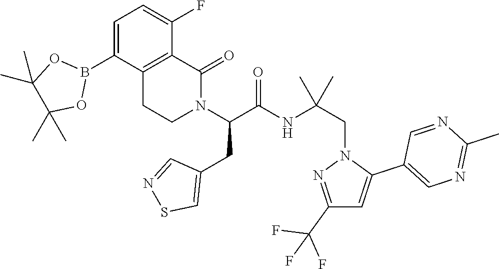 | 728.4 (M + H) |

^{a}PdCl₂(dppf)-CH₂Cl₂ adduct as the catalyst, 1,4-dioxane as the solvent
^{b}(2-Dicyclohexylphosphino-2′,6′-dimethoxybiphenyl)[2-(2′-amino-1,1′-biphenyl)]palladium(II) methanesulfonate as the catalyst, DMF as the solvent

### Preparation 380

### Methyl (E)-2-(3-((tert-butyldimethylsilyl)oxy)prop-1-en-1-yl)-3-chloro-6-fluorobenzoate

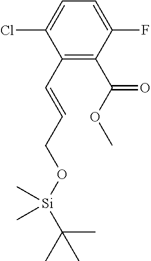 [SMILES: COC(=O)c1c(F)ccc(Cl)c1/C=C/CO[Si](C)(C)C(C)(C)C]

To a solution of methyl 2-bromo-3-chloro-6-fluorobenzoate (2.89 g, 95% purity, 10.3 mmol) in 1,4-dioxane (120 mL) and H₂O (24 mL) were added (E)-tert-butyldimethyl {[3-(4,4,5,5-tetramethyl-1,3,2-dioxaborolan-2-yl)allyl]oxy}silane (3.68 g, 12.3 mmol), sodium carbonate (3.26 g, 30.8 mmol) and PdCl₂(dppf) (920 mg, 1.26 mmol). The reaction mixture was evacuated and backfilled with N₂×3, then gradually heated to 100° C. and stirred for 3 h. The reaction mixture was diluted with EtOAc (50 mL), filtered through a short pad of Na₂SO₄, and concentrated under reduced pressure. Column chromatography (SiO₂, 20:1 hexanes:EtOAc) provided the title compound (3.367 g, 80% purity, 73%) as a light-yellow oil. ES-MS m/z 226.9/229.0 (M-TBSO) (Cl^{35/37}).

### Preparation 381

### Methyl 2-(3-((tert-butyldimethylsilyl)oxy)propyl)-3-chloro-6-fluorobenzoate

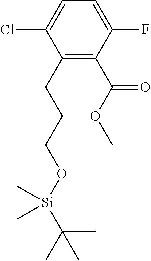 [SMILES: COC(=O)c1c(F)ccc(Cl)c1CCCO[Si](C)(C)C(C)(C)C]

A solution of methyl (E)-2-(3-((tert-butyldimethylsilyl)oxy)prop-1-en-1-yl)-3-chloro-6-fluorobenzoate (3.08 g, 80% purity, 6.87 mmol) and 1,1′-bis(di-isopropylphosphino)ferrocene (1,5-cyclootadiene)rhodium tetrafluoroborate (290 mg, 382 μmol) in MeOH (150 mL) and THF (15 mL) was degassed with N₂×3, then evacuated and backfilled with H₂×3. The mixture was stirred at 19° C. for 16 h under 50 psi H₂, then concentrated under reduced pressure. Column chromatography (SiO₂, 20:1 hexanes:EtOAc) provided the title compound (2.38 g, 70% purity, 67%) as a colorless oil. ES-MS m/z 361.0/363.0 (M+H) (Cl^{35/37}).

### Preparation 382

### 3-Chloro-6-fluoro-2-(3-hydroxypropyl)benzoic acid

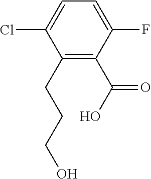 [SMILES: O=C(O)c1c(F)ccc(Cl)c1CCCO]

To a mixture of methyl 2-(3-((tert-butyldimethylsilyl)oxy)propyl)-3-chloro-6-fluorobenzoate (1.0 g, 70% purity, 1.9 mmol) in THF (9 mL) and MeOH (9 mL) was added lithium hydroxide monohydrate (0.80 g, 19 mmol) in H₂O (6 mL) at 13° C. The reaction mixture was stirred at 70° C. for 16 h, concentrated under reduced pressure, diluted with H₂O (30 mL), washed with DCM (30 mL) and the organic layer was discarded. The aqueous layer was adjusted to about pH 1 with 1 M HCl and extracted with EtOAc (50 mL×2). The combined organic layers were washed with saturated aqueous NaCl (50 mL), dried over Na₂SO₄, filtered, and concentrated under reduced pressure. Column chromatography (SiO₂, 3-4% MeOH in DCM) afforded the title compound (410 mg, 90% purity, 82%) as a yellow solid. ES-MS m/z 232.8/234.7 (M+H) (Cl^{35/37}).

### Preparation 383

### 3-Chloro-2-(3-chloropropyl)-6-fluorobenzoyl chloride

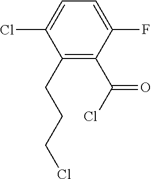 [SMILES: O=C(Cl)c1c(F)ccc(Cl)c1CCCCl]

To a mixture of 3-chloro-6-fluoro-2-(3-hydroxypropyl)benzoic acid (149 mg, 90% purity, 576 μmol) in SOCl₂ (4 mL) was added one drop of DMF at 8° C. The reaction mixture was degassed and purged with N₂×3, stirred at 70° C. for 12 h, and concentrated in vacuo. The residue was co-evaporated with toluene (1 mL) to provide the title compound (200 mg, 75% purity, 97%) as yellow oil. A sample of the corresponding methyl ester was prepared by quenching the crude product with excess MeOH: ES-MS m/z 264.8 (M+H).

### Preparation 384

### (R)-3-Chloro-2-(3-chloropropyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-6-fluorobenzamide

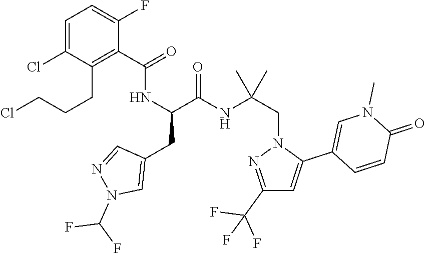 [SMILES: Cn1cc(-c2cc(C(F)(F)F)nn2CC(C)(C)NC(=O)[C@@H](Cc2cnn(C(F)F)c2)NC(=O)c2c(F)ccc(Cl)c2CCCCl)ccc1=O]

To a mixture of (R)-2-amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide hydrochloride (361 mg, 671 μmol) and TEA (950 μL, 6.82 mmol) in THF (6 mL) was added 3-chloro-2-(3-chloropropyl)-6-fluorobenzoyl chloride (200 mg, 75% purity, 557 μmol) in THF (6 mL) at 0° C. The reaction mixture was stirred at 15° C. for 1 h, diluted with EtOAc (50 mL), washed with H₂O (30 mL×2) and saturated aqueous NaCl (30 mL×2), dried over Na₂SO₄, filtered, and concentrated under reduced pressure. Column chromatography (SiO₂, 3-4% MeOH in DCM) provided the title compound (410 mg, 90% purity, 90%) as a white solid. ES-MS m/z 733.9 (M+H).

### Preparation 385

### (R)-2-(6-Chloro-9-fluoro-1-oxo-1,3,4,5-tetrahydro-2H-benzo[c]azepin-2-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

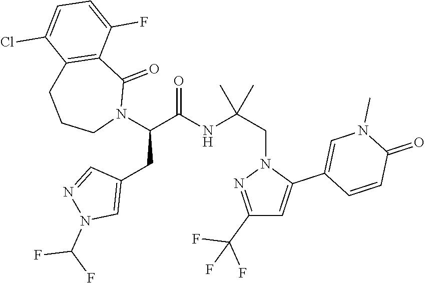 [SMILES: Cn1cc(-c2cc(C(F)(F)F)nn2CC(C)(C)NC(=O)[C@@H](Cc2cnn(C(F)F)c2)N2CCCc3c(Cl)ccc(F)c3C2=O)ccc1=O]

To a mixture of (R)-3-chloro-2-(3-chloropropyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-6-fluorobenzamide (410 mg, 90% purity, 502 μmol) in ACN (12 mL) was added Cs₂CO₃ (503 mg, 1.54 mmol). The reaction mixture was stirred at 80° C. for 5 h, diluted with H₂O (50 mL) and extracted with EtOAc (50 mL×2). The combined organics were washed with saturated aqueous NaCl (50 mL×2), dried over Na₂SO₄, filtered, and concentrated under reduced pressure. The residue was purified by prep-HPLC (YMC Triart C18, 250*30 mm*5 μm; gradient 44-74% ACN in 10 mM aqueous NH₄HCO₃), the fractions were concentrated under reduced pressure to remove ACN, and the aqueous residue was extracted with EtOAc (30 mL×2). The combined organics were washed with saturated aqueous NaCl (50 mL), dried over Na₂SO₄, filtered, and concentrated under reduced pressure. The residue was lyophilized to provide the title compound (260 mg, 95% purity, 70%) as a white solid. ES-MS m/z 698.0 (M+H).

### Preparation 386

### (R)-2-Amino-3-bromo-N-(3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)-6-fluorobenzamide

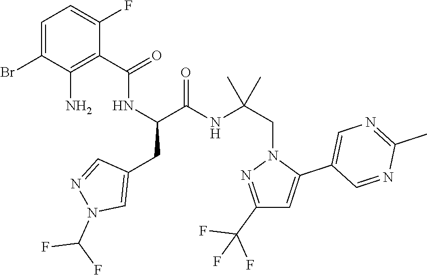 [SMILES: Cc1ncc(-c2cc(C(F)(F)F)nn2CC(C)(C)NC(=O)[C@@H](Cc2cnn(C(F)F)c2)NC(=O)c2c(F)ccc(Br)c2N)cn1]

In a dry 40-mL sealed tube, a solution of (R)-2-amino-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide hydrochloride (411 mg, 92 wt %, 723 μmol), 2-amino-3-bromo-6-fluorobenzoic acid (178 mg, 761 μmol), and HATU (425 mg, 1.12 mmol) in DMA (5 mL) was added DIEA (400 μL, 2.30 mmol). The mixture was stirred at 20° C. for 2 h. The mixture was diluted with water (20 mL) and extracted with EtOAc (30 mL×2). The combined organic layers were washed with saturated aqueous NaCl (30 mL×3), dried over Na₂SO₄, filtered, and concentrated under reduced pressure. Column chromatography (SiO₂, gradient 0-4% MeOH in DCM) provided the title compound (553 mg, 75% purty, 82%) as a yellow solid. ES-MS m/z 702.0/703.9 (M+H).

### Preparation 387

### (R)-2-(8-Bromo-5-fluoro-4-oxoquinazolin-3 (4H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide

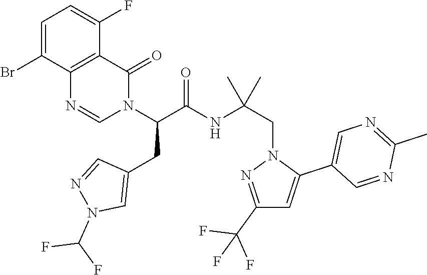 [SMILES: Cc1ncc(-c2cc(C(F)(F)F)nn2CC(C)(C)NC(=O)[C@@H](Cc2cnn(C(F)F)c2)n2cnc3c(Br)ccc(F)c3c2=O)cn1]

In a 40-mL sealed tube, a solution of (R)-2-amino-3-bromo-N-(3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)-6-fluorobenzamide (232 mg, 75 wt %, 248 μmol) in EtOH (0.5 mL) was treated with triethoxymethane (3.00 mL, 18.0 mmol) and acetic acid (100 μL, 1.75 mmol). The reaction mixture was stirred at 80° C. for 3 days. The reaction mixture was concentrated under reduced pressure to remove solvent. The residue was diluted with water (5 mL) and extracted with EtOAc (10 mL×2). The combined organic layers were washed with saturated aqueous NaCl (10 mL×2), dried over Na₂SO₄, filtered, and concentrated under reduced pressure. Column chromatography (SiO₂, gradient 0-4% MeOH in DCM) provided the title compound (148 mg, 80% purity, 67%) as a yellow oil. ES-MS m/z 712.0/713.9 (M+H).

### Preparation 388

### (R)-(3-(3-(1-(Difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)-5-fluoro-4-oxo-3,4-dihydroquinazolin-8-yl)boronic acid

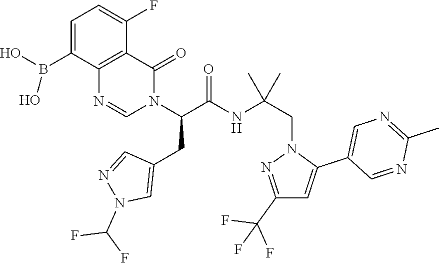 [SMILES: Cc1ncc(-c2cc(C(F)(F)F)nn2CC(C)(C)NC(=O)[C@@H](Cc2cnn(C(F)F)c2)n2cnc3c(B(O)O)ccc(F)c3c2=O)cn1]

In a N₂-filled glovebox, (R)-2-(8-bromo-5-fluoro-4-oxoquinazolin-3 (4H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide (148 mg, 80 wt %, 166 μmol), 4,4,4′,4′,5,5,5′,5′-octamethyl-2,2′-bi (1,3,2-dioxaborolane) (356 mg, 1.40 mmol), potassium acetate (53 mg, 0.54 mmol), and SPhos Pd G3 (18 mg, 21 μmol) were added into a dry 40-mL sealed tube. DMF (4 mL) was added to the mixture, then the vial was sealed and taken out of the glovebox. The reaction mixture was stirred at 90° C. for 16 h. The mixture was diluted with water (10 mL) and extracted with EtOAc (10 mL×2). The combined organic layers were washed with saturated aqueous NaCl (15 mL×3), dried over Na₂SO₄, filtered, and concentrated under reduced pressure. Column chromatography (SiO₂, gradient 0-6% MeOH in DCM) provided the title compound (44 mg, 82% purty, 32%) as a yellow oil. ES-MS m/z 678.1 (M+H).

### Preparation 389

### 2-Bromo-3-chloro-N-cyclopropyl-6-fluoro-N-(4-methoxybenzyl)benzamide

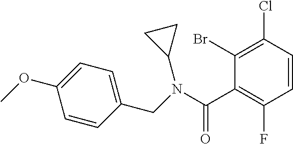 [SMILES: COc1ccc(CN(C(=O)c2c(F)ccc(Cl)c2Br)C2CC2)cc1]

In a 20-mL vial, 2-bromo-3-chloro-6-fluorobenzoic acid (500 mg, 1.97 mmol) and N-(4-methoxybenzyl)cyclopropylamine (368 mg, 2.08 mmol) were dissolved in DMF (10 mL). Then, TEA (420 μL, 3.01 mmol) was added followed by HATU (831 mg, 2.19 mmol), and the reaction was stirred overnight at RT. The mixture was quenched via slow addition to rapidly stirring water (200 mL), and the resulting suspension was stirred overnight. The quenched mixture was filtered, and the aqueous filtrate was discarded. The solids were dissolved in methanol and filtered, then the filtrate was concentrated under reduced pressure. Column chromatography (SiO₂, gradient 0-100% EtOAc in heptane) provided the title compound (750 mg, 98% purty, 90%) as a yellow oil. ES-MS m/z 412.0/414.0 (M+H).

### Preparation 390

### rac-(1aR,7bR)-7-Chloro-4-fluoro-2-(4-methoxybenzyl)-1,1a,2,7b-tetrahydro-3H-cyclopropa[c]isoquinolin-3-one

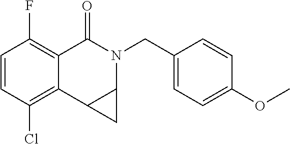 [SMILES: COc1ccc(CN2C(=O)c3c(F)ccc(Cl)c3C3CC32)cc1]

A solution of 2-bromo-3-chloro-N-cyclopropyl-6-fluoro-N-(4-methoxybenzyl)benzamide (750.3 mg, 1.818 mmol), K₃PO₄ (656 mg, 1.70 Eq, 3.09 mmol), 1-adamantane carboxylic acid (66.6 mg, 369 μmol), bis(dibenzylideneacetone)palladium (52.5 mg, 91.3 μmol), and bis(1,1-dimethylethyl)(methyl)phosphine tetrafluoroborate (47.9 mg, 193 μmol) in toluene (7.3 mL) was sparged with argon for ˜5 min and then stirring was initiated and the reaction was heated to 135° C. After 38 h, the reaction was cooled to RT, diluted with EtOAc and filtered through a plug of diatomaceous earth. The filtrate was concentrated under reduced pressure and purified 2× by silica gel chromatography using a gradient of 0-100% EtOAc in heptane to afford the title compound (401.6 mg, 88% purity, 59% yield) as an orange oil. ES-MS m/z 332.2, 334.2 (M+H).

### Preparation 391

### rac-(1aR,7bR)-7-Chloro-4-fluoro-1,1a,2,7b-tetrahydro-3H-cyclopropa[c]isoquinolin-3-one

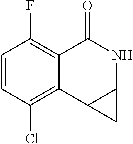 [SMILES: O=C1NC2CC2c2c(Cl)ccc(F)c21]

A solution of rac-(1aR,7bR)-7-chloro-4-fluoro-2-(4-methoxybenzyl)-1,1a,2,7b-tetrahydro-3H-cyclopropa[c]isoquinolin-3-one (401.6 mg, 1.210 mmol) and anisole (660 μL, 6.04 mmol) in TFA (2 mL) was prepared, stirring was initiated, and the mixture was heated to 65° C. After 18.75 h, the mixture was cooled to RT and then concentrated under reduced pressure. The resulting solids were triturated with ˜10 mL of MTBE and then collected by filtration, using additional MTBE to aid transfer and rinse the filter cake. The filter cake was then collected to afford the title compound (196.5 mg, 96% purity, 74% yield) as a white solid. ES-MS m/z 212.0, 214.0 (M+H).

### Preparation 392

### rac-Ethyl 2-((1aR,7bR)-7-chloro-4-fluoro-3-oxo-1,1a,3,7b-tetrahydro-2H-cyclopropa[c]isoquinolin-2-yl)acetate

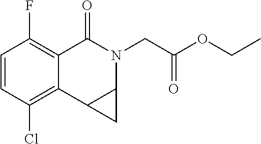 [SMILES: CCOC(=O)CN1C(=O)c2c(F)ccc(Cl)c2C2CC21]

A solution of rac-(1aR,7bR)-7-chloro-4-fluoro-1,1a,2,7b-tetrahydro-3H-cyclopropa[c]isoquinolin-3-one (196.5 mg, 928.6 μmol) and Cs₂CO₃ (396.9 mg, 1.218 mmol) in DMF (3 mL) was treated with ethyl bromoacetate (140 μL, 1.26 mmol). Then stirring was initiated and the reaction was heated to 60° C. After 1 h, the reaction was removed from heating and quenched by slowly adding it into a rapidly stirring solution of H₂O (˜100 mL) and HCl (1.5 mL of a 1 M aq. soln.). The resulting suspension was stirred for ˜20 min and then the solids were collected by filtration. The filter cake was washed with water, then it was collected and purified by silica gel chromatography using a gradient of 0-100% EtOAc in heptane to afford the title compounds (243.9 mg, 96% purity, 85% yield) as a white solid. ES-MS m/z 298.2, 300.2 (M+H).

### Preparation 393

### rac-Methyl-2-((cis)-7-chloro-4-fluoro-3-oxo-1,1a,3,7b-tetrahydro-2H-cyclopropa[c]isoquinolin-2-yl)-3-(1-methyl-1H-pyrazol-4-yl)propanoate (mixture of isomers)

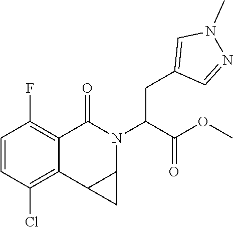 [SMILES: COC(=O)C(Cc1cnn(C)c1)N1C(=O)c2c(F)ccc(Cl)c2C2CC21]

A mixture of rac-ethyl 2-((1aR,7bR)-7-chloro-4-fluoro-3-oxo-1,1a,3,7b-tetrahydro-2H-cyclopropa[c]isoquinolin-2-yl)acetate (243.9 mg, 819.3 μmol) and 4-(bromomethyl)-1-methyl-1H-pyrazole hydrobromide (236.2 mg, 922.9 μmol) was placed under an atmosphere of argon and suspended in THF (8.2 mL). Stirring was initiated, and the mixture was cooled to −78° C. Then NaHMDS (940 μL of a 2 M soln. in THF, 1.88 mmol) was added in a single portion, after which the cooling bath was immediately removed to allow the reaction to gradually warm to RT. After 30 min, the reaction was quenched by the addition of 900 μL of MeOH. The reaction was then diluted with EtOAc and sat. aq. NH₄Cl. The organic layer was removed, and the aqueous layer was extracted with EtOAc (2×). The combined organic layers were then washed with saturated aqueous NaCl, dried over Na₂SO₄, filtered, concentrated under reduced pressure, and purified by silica gel chromatography using a gradient of 0-100% of a 3:1 EtOAc:EtOH soln. in heptane to afford the title compound (179.6 mg, 88% purity, 51% yield, ˜55:45 ratio of diastereomers) as a colorless oil. ES-MS m/z 378.2, 380.2 (M+H).

### Preparation 394

### rac-(R)-2-((1aS,7bS)-7-chloro-4-fluoro-3-oxo-1,1a,3,7b-tetrahydro-2H-cyclopropa[c]isoquinolin-2-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-3-(1-methyl-1H-pyrazol-4-yl)propanamide (Mixture of Isomers)

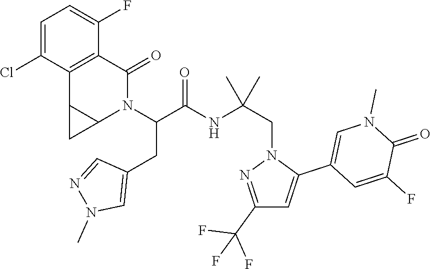 [SMILES: Cn1cc(CC(C(=O)NC(C)(C)Cn2nc(C(F)(F)F)cc2-c2cc(F)c(=O)n(C)c2)N2C(=O)c3c(F)ccc(Cl)c3C3CC32)cn1]

A solution of rac-methyl-2-((cis)-7-chloro-4-fluoro-3-oxo-1,1a,3,7b-tetrahydro-2H-cyclopropa[c]isoquinolin-2-yl)-3-(1-methyl-1H-pyrazol-4-yl)propanoate (179.6 mg, 470.0 μmol) in THF (3.5 mL) was cooled to 0° C., stirring was initiated, and then LiOH (700 μL of a 2 M Aq. soln., 40 mmol) was added. After 3.7 h, the reaction was quenched by the addition of HCl (1.5 mL of a 1 M aq. soln.), which brought the pH to ˜4. The reaction was then diluted with water and DCM. The organic layer was removed, and the aqueous layer was extracted 3× with a 3:1 solution of CHCl₃:IPA. The combined organic layers were then dried over Na₂SO₄, filtered, and concentrated under reduced pressure to afford the crude acid as a colorless waxy solid. To this was added 1-(5-(5-fluoro-1-methyl-6-oxo-1,6-dihydropyridin-3-yl)-3-(trifluoromethyl)-1H-pyrazol-1-yl)-2-methylpropan-2-aminium chloride (237.3 mg, 643.5 μmol). The solids were suspended in DMF (2.5 mL) and then TEA (320 μL, 2.30 mmol) was added. The mixture was allowed to stir for ˜3 min, then HATU (271.8 mg, 714.8 μmol) was added, and the reaction was allowed to proceed at RT. After 14.3 h the reaction was halted by slowly adding it to ˜150 mL of rapidly stirring water. The resulting suspension was allowed to stir for ˜1.5 h, after which the solids were collected via filtration and rinsed with a large volume of water. The filter cake was then collected and purified by silica gel chromatography using a gradient of 0-100% of a solution of 3:1 EtOAc:EtOH in heptane to afford the title compound (200.1 mg, 98% purity, 61% yield of an ˜2:1 mixture of diastereomers) as a white solid. ES-MS m/z 678.2, 680.2 (M+H).

### Preparation 395

### 3-Iodo-4,5-dihydro-7H-thieno[2,3-c]pyran-7-one

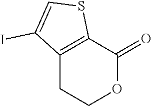 [SMILES: O=C1OCCc2c(I)csc21]

To a solution of 4,5-dihydro-7H-thieno[2,3-c]pyran-7-one (394 mg, 2.56 mmol) in DCE (5 mL) was added I₂ (588 mg, 2.32 mmol) and (diacetoxy)iodobenzene (756 mg, 2.35 mmol). The mixture was stirred at 65° C. for 2 h, after which it was quenched by the addition of sat. aq. Na₂S₂O₃ (10 mL). The mixture was extracted with DCM (2×10 mL), then the combined organic layers were washed with saturated aqueous NaCl (2×10 mL), dried over Na₂SO₄, filtered, concentrated under reduced pressure, and purified by silica gel chromatography using a gradient of 0-20% EtOAc in hexanes to afford the title compound (272 mg, 95% purity, 36% yield) as a white solid. ES-MS m/z 280.9 (M+H).

### Preparation 396

### (R)-3-(2-Chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-4-iodothiophene-2-carboxamide

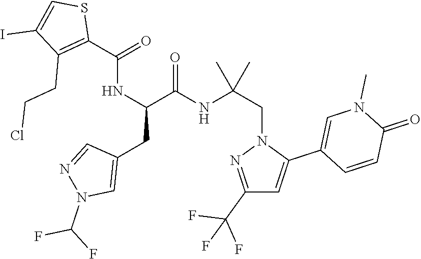 [SMILES: Cn1cc(-c2cc(C(F)(F)F)nn2CC(C)(C)NC(=O)[C@@H](Cc2cnn(C(F)F)c2)NC(=O)c2scc(I)c2CCCl)ccc1=O]

To a solution of 3-iodo-4,5-dihydro-7H-thieno[2,3-c]pyran-7-one (272 mg, 95 wt %, 923 μmol) in SOCl₂ (3.0 mL, 41.11 mmol) was added DMF (50 μL, 0.65 mmol). The reaction mixture was degassed and purged with N₂ 3× and then stirring was initiated under an atmosphere of N₂ and the reaction was heated to 80° C. for 16 h. The crude reaction mixture was then concentrated to dryness under reduced pressure to afford the crude dichloride (420 mg, 37% purity, 50% yield) as a yellow oil, which was dissolved in THF (3 mL). In a separate vessel, a solution of (R)-2-amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide hydrochloride (210 mg, 390 μmol) in THF (4 mL) was treated with TEA (200 μL, 1.43 mmol). Then the solution of the dichloride described above was added to the mixture and the reaction was stirred at 20° C. for 1 h. The mixture was then diluted with H₂O (10 mL) and extracted with EtOAc (2×10 mL). The combined organic layers were dried over Na₂SO₄, filtered, concentrated under reduced pressure, and purified by silica gel chromatography using a gradient of 0-4% MeOH in DCM to afford the title compound (212 mg, 88% purity, 60% yield) as a white solid. ES-MS m/z 799.9, 801.9 (M+H).

### Preparation 397

### (R)-3-(1-(Difluoromethyl)-1H-pyrazol-4-yl)-2-(3-iodo-7-oxo-4,7-dihydrothieno[2,3-c]pyridin-6 (5H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

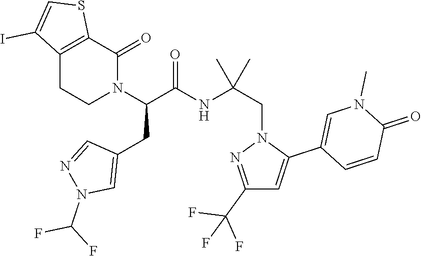 [SMILES: Cn1cc(-c2cc(C(F)(F)F)nn2CC(C)(C)NC(=O)[C@@H](Cc2cnn(C(F)F)c2)N2CCc3c(I)csc3C2=O)ccc1=O]

To a solution of (R)-3-(2-chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-4-iodothiophene-2-carboxamide (212 mg, 88 wt %, 233 μmol) in ACN (5 mL) was added Cs₂CO₃ (241 mg, 740 μmol), and the resulting mixture was stirred at 20° C. for 2 h. The reaction was then diluted with H₂O (5 mL) and extracted with EtOAc (2×10 mL). The combined organic layers were dried over Na₂SO₄, filtered, concentrated under reduced pressure, and purified by silica gel chromatography using a gradient of 0-4% MeOH in DCM to afford the title compound (191 mg, 89% purity, 95% yield) as a white solid. ES-MS m/z 764.0 (M+H).

### Preparation 398

### (R)-(6-(3-(1-(Difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-7-oxo-4,5,6,7-tetrahydrothieno[2,3-c]pyridin-3-yl)boronic acid

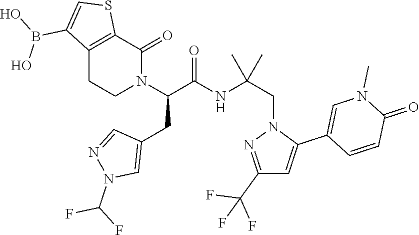 [SMILES: Cn1cc(-c2cc(C(F)(F)F)nn2CC(C)(C)NC(=O)[C@@H](Cc2cnn(C(F)F)c2)N2CCc3c(B(O)O)csc3C2=O)ccc1=O]

A mixture of (R)-3-(1-(difluoromethyl)-1H-pyrazol-4-yl)-2-(3-iodo-7-oxo-4,7-dihydrothieno[2,3-c]pyridin-6 (5H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (191 mg, 1 eq, 223 μmol), 4,4,4′,4′,5,5,5′,5′-octamethyl-2,2′-bi (1,3,2-dioxaborolane) (463 mg, 8.19 eq, 1.82 mmol), potassium acetate (68 mg, 3.1 eq, 0.69 mmol) and (2-dicyclohexylphosphino-2′,6′-dimethoxybiphenyl)[2-(2′-amino-1,1′-biphenyl)]palladium(II) methanesulfonate (24 mg, 0.12 eq, 27 μmol) in DMF (4 mL) was heated to 90° C. for 16 h under an N₂ atmosphere. After cooling to RT, the reaction was diluted with H₂O (10 mL) and extracted with EtOAc (10 mL×2). The combined organic layer was washed with saturated aqueous NaCl (15 mL×3), dried over anhydrous Na₂SO₄, filtered and concentrated under reduced pressure. The residue was purified by column chromatography (SiO2, 0-6% MeOH/DCM gradient) to afford the title compound (122 mg, 92% purity, 74% yield) as a white solid. ES-MS m/z 682.0 (M+H).

### Preparation 399

### 6-Fluoro-1-methyl-5-(4,4,5,5-tetramethyl-1,3,2-dioxaborolan-2-yl)-1H-indazole

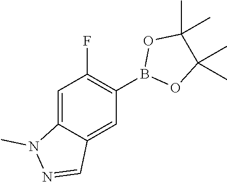

To a mixture of 5-bromo-6-fluoro-1-methyl-1H-indazole (200 mg, 873 μmol) and 4,4,4′,4′,5,5,5′,5′-octamethyl-2,2′-bi (1,3,2-dioxaborolane) (273 mg, 1.08 mmol) in 1,4-dioxane (3 mL) were added KOAc (265 mg, 2.70 mmol) and PdCl₂(dppf) (64.4 mg, 88.0 μmol). The mixture was degassed and purged with N₂ 3×, then the reaction mixture was gradually heated to 100° C. and stirred at that temperature for 16 h. The reaction was then cooled to RT, concentrated under reduced pressure, and purified by silica gel chromatography using a gradient of 0-35% EtOAc in hexanes to afford the title compound (182 mg, 64%) as a yellow solid. ES-MS m/z 276.7 (M+H).

### Preparation 400

### rac-tert-Butyl (1-(1-(1-(2-fluorophenyl)ethyl)-4-(trifluoromethyl)-1H-imidazol-2-yl)-2-methylpropan-2-yl)carbamate

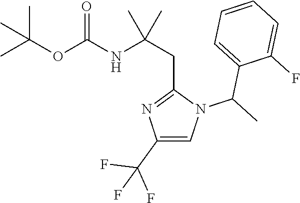 [SMILES: CC(c1ccccc1F)n1cc(C(F)(F)F)nc1CC(C)(C)NC(=O)OC(C)(C)C]

To a solution of tert-butyl (2-methyl-1-(4-(trifluoromethyl)-1H-imidazol-2-yl)propan-2-yl)carbamate (75 mg, 0.24 mmol) in DMF (0.51 mL) was added potassium carbonate (118 mg, 0.85 mmol). The mixture was stirred at RT for 30 min, then 1-(1-bromoethyl)-2-fluorobenzene (87 mg, 0.43 mmol) was added. An additional 0.5 mL DMF was added and heated to 60° C. The mixture was stirred at 60° C. for 15 h, then cooled to RT. The mixture was diluted with DCM, transferred to a 10 mL vial, and concentrated under N₂. The residue was diluted with water (5 mL) and DCM (5 mL). The mixture was filtered through an SPE phase separator cartridge, then rinsed with DCM (5 mL). The organic phase was concentrated under N₂. The residue was purified by reverse phase HPLC using a gradient of 50 to 100% ACN in aq. NH₄HCO₃ to provide the title compound (81 mg, 77%). ES-MS m/z 430.2 (M+H).

### Preparation 401

### rac-1-(1-(1-(2-Fluorophenyl)ethyl)-4-(trifluoromethyl)-1H-imidazol-2-yl)-2-methylpropan-2-amine dihydrochloride

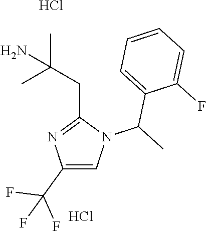 [SMILES: Cl ; CC(c1ccccc1F)n1cc(C(F)(F)F)nc1CC(C)(C)N ; Cl]

To rac-tert-butyl (1-(1-(1-(2-fluorophenyl)ethyl)-4-(trifluoromethyl)-1H-imidazol-2-yl)-2-methylpropan-2-yl)carbamate (80 mg, 0.19 mmol) in a 40 mL vial was added DCM (7 mL) followed by HCl in dioxane (4 M, 0.47 mL, 1.9 mmol). The vial was shaken at RT for 8 h, then concentrated under a stream of N₂. The residue was suspended in DCM and ACN, then re-concentrated under a stream of N₂. The residue was placed in a vacuum oven at 50° C. overnight to provide the title compound (73 mg, 97% yield) as a white solid. ES-MS m/z 330.2 (M+H).

### Preparation 402

### tert-Butyl (R)-2-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)hydrazine-1-carboxylate

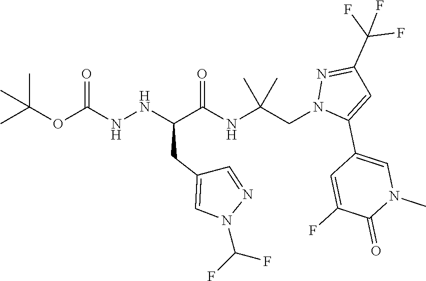 [SMILES: Cn1cc(-c2cc(C(F)(F)F)nn2CC(C)(C)NC(=O)[C@@H](Cc2cnn(C(F)F)c2)NNC(=O)OC(C)(C)C)cc(F)c1=O]

To a solution of (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (300 mg, 0.578 mmol) in THF (5.8 mL) was added saturated aqueous sodium bicarbonate solution (5.8 mL). The resulting white slurry was stirred vigorously for 10 min. 2-(tert-Butyl) 3,3-diethyl 1,2-oxaziridine-2,3,3-tricarboxylate (167 mg, 0.578 mmol) was added and the reaction mixture was stirred at RT for 2 h. The reaction mixture was treated with ethylenediamine (154 μL, 2.30 mmol) to quench ketone byproduct and the mixture was stirred for 5 min. The reaction mixture was partitioned with EtOAc (15 mL), the layers separated, and the aqueous layer extracted with EtOAc (10 mL×2). The combined organic layers were concentrated under a stream of nitrogen. The residue was purified by column chromatography (SiO2, 0-7% MeOH/DCM gradient) to afford the title compound (328 mg, 90%) as a glassy, colorless solid. ES-MS m/z 535.4 (M+H).

### Preparation 403

### (R)-3-(1-(Difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-hydrazineylpropanamide

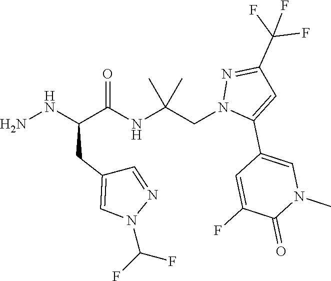 [SMILES: Cn1cc(-c2cc(C(F)(F)F)nn2CC(C)(C)NC(=O)[C@@H](Cc2cnn(C(F)F)c2)NN)cc(F)c1=O]

In a 100-mL flask, a solution of tert-butyl (R)-2-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)hydrazine-1-carboxylate (328 mg, 0.518 mmol) in MeOH (2.6 mL) was treated with HCl in dioxane (4.0 M, 1.30 mL, 5.20 mmol). The resulting light-yellow solution was stirred at RT for 26 h. The reaction mixture was concentrated under reduced pressure, then the residue was partitioned between DCM (15 mL) and saturated aqueous sodium bicarbonate solution (15 mL). The layers were separated and the aqueous layer was extracted with 4:1 DCM/IPA (10 mL×2). The combined organic layers were dried over anhydrous Na₂SO₄ and filtered. The filtrate was concentrated under a stream of nitrogen to afford the title compound (256 mg, 92%) as a white solid. ES-MS m/z 505.2 (M+H).

### Preparation 404

### (R)-2-((tert-Butoxycarbonyl)amino)-3-(1-(methyl-d3)-1H-pyrazol-4-yl)propanoic acid

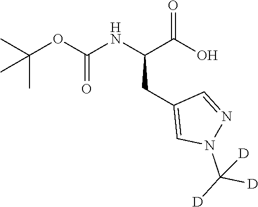 [SMILES: [2H]C([2H])([2H])n1cc(C[C@@H](NC(=O)OC(C)(C)C)C(=O)O)cn1]

To a 100 mL RBF was added methyl (R)-2-((tert-butoxycarbonyl)amino)-3-(1-(methyl-d3)-1H-pyrazol-4-yl)propanoate (1.31 g, 4.57 mmol), THF (20 mL), and water (5 mL). Then added LiOH (219 mg, 9.15 mmol) and sufficient MeOH to provide a homogeneous solution. The solution was stirred at an ambient temperature for 2 h, then quenched with 1 N HCl and extracted with EtOAc. The organic phase was dried over MgSO₄, filtered, and concentrated under reduced pressure to provide the title compound as a white solid (789 mg, 62%). ES/MS m/z 273.2 (M+H).

### Preparation 405

### tert-Butyl (1-hydroxy-2-methylpropan-2-yl-1,1-d2) carbamate

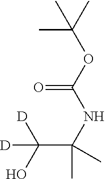

To an oven-dried flask was added methyl 2-(tert-butoxycarbonylamino)-2-methylpropanoate (5.0 g, 23 mmol). Diethyl ether (75 mL) was added. The flask was evacuated and backfilled with N₂. This solution was cooled in an ice bath. To a separate oven-dried flask was added lithium aluminum deuteride (1.4 g, 33 mmol). The flask was evacuated and backfilled with N₂. Diethyl ether (40 mL) was added, and the mixture was stirred rapidly. When the substrate solution reached 5° C. (internal), the suspension of LiAlD₄ was added by cannula. The addition was kept at a rate such that the internal temperature did not exceed 11° C. The ice bath was removed, and the mixture was stirred for 30 min. MTBE (40 mL) was added, and the mixture was again cooled in the ice bath. When the temperature reached 5° C., the reaction was quenched by the slow addition of water (1.4 mL), followed by 5 M NaOH (1.4 mL), followed by additional water (4.2 mL). The ice bath was then removed, and the mixture was left to stir overnight. The reaction mixture was filtered through a pad of MgSO₄. The filtrate was concentrated under reduced pressure. The residue was then dried at 60° C. under vacuum to provide the title compound as a white solid (4.08 g, 93%). 1H-NMR (CDCl₃) δ 4.70 (s, 1H), 1.44 (s, 9H), 1.26 (s, 6H).

### Preparation 406

### tert-Butyl 4,4-dimethyl-1,2,3-oxathiazolidine-3-carboxylate-5,5-d2 2-oxide

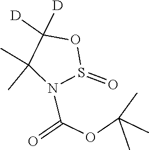 [SMILES: [2H]C1([2H])OS(=O)N(C(=O)OC(C)(C)C)C1(C)C]

To a solution of thionyl chloride (3.93 mL, 53.3 mmol) in ACN (50 mL) at −40° C. was added tert-butyl (1-hydroxy-2-methylpropan-2-yl-1,1-d2)carbamate (4.08 g, 21.3 mmol) in ACN (50 mL). After 5 min, pyridine (6.04 mL, 74.7 mmol) was added dropwise. The reaction was stirred for 30 min, then warmed to 0° C., then stirred another 30 min, then warmed to RT over 15 min. The mixture was then diluted with EtOAc (200 mL). The mixture was washed with 1 N HCl (aq) (100 mL) and Sat. NaHCO₃ (100 mL). The organic phase was dried over MgSO₄, filtered and concentrated under reduced pressure to provide the title compound as a brown oil (4.28 g, 85%). 1H-NMR (CDCl₃) δ 1.61 (s, 3H), 1.53 (s, 9H), 1.41 (s, 3H).

### Preparation 407

### tert-Butyl 4,4-dimethyl-1,2,3-oxathiazolidine-3-carboxylate-5,5-d2 2,2-dioxide

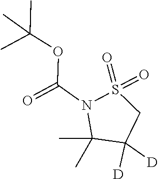 [SMILES: [2H]C1([2H])CS(=O)(=O)N(C(=O)OC(C)(C)C)C1(C)C]

The suspension of tert-butyl 4,4-dimethyl-1,2,3-oxathiazolidine-3-carboxylate-5,5-d2 2-oxide (4.28 g, 18.0 mmol) in ACN (180 mL) and water (90.2 mL) was cooled in an ice bath. Sodium metaperiodate (5.01 g, 23.4 mmol) and ruthenium trichloride (74.8 mg, 361 μmol) were added. The resulting mixture was allowed to warm to RT and stirred for 1 h. The mixture was diluted with ether (100 mL) and water. The layers were separated. The aqueous layer was extracted with ether. The ether layers were combined, washed with water then saturated aqueous NaCl (2×), dried over MgSO₄, and filtered. The filtrate was concentrated under reduced pressure to obtain the title compound (4.39 g, 96%) as a white solid. 1H-NMR (CDCl₃) δ 1.60 (s, 6H), 1.57 (s, 9H).

### Preparation 408

### tert-Butyl (1-(5-bromo-3-(trifluoromethyl)-1H-pyrazol-1-yl)-2-methylpropan-2-yl-1,1-d2) carbamate

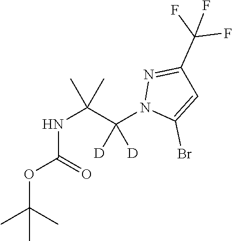 [SMILES: [2H]C([2H])(n1nc(C(F)(F)F)cc1Br)C(C)(C)NC(=O)OC(C)(C)C]

The title compound was prepared essentially as described in Preparation 41 using tert-butyl 4,4-dimethyl-1,2,3-oxathiazolidine-3-carboxylate-5,5-d2 2,2-dioxide (stirring at 95° C. for 3 h) and isolated as the second eluting regioisomer (major isomer) when purified on silica with a gradient of 0-10% MTBE in hexanes. ES-MS m/z (⁷⁹Br/⁸¹Br) 332.0/334.0 (M-tBu+H).

### Preparation 409

### 1-(5-Bromo-3-(trifluoromethyl)-1H-pyrazol-1-yl)-2-methylpropan-1,1-d2-2-amine hydrochloride

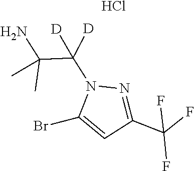 [SMILES: Cl ; [2H]C([2H])(n1nc(C(F)(F)F)cc1Br)C(C)(C)N]

A solution of tert-butyl (1-(5-bromo-3-(trifluoromethyl)-1H-pyrazol-1-yl)-2-methylpropan-2-yl-1,1-d2) carbamate (4.74 g, 12.2 mmol) in 1,4-dioxane (25 mL) was treated with 4M HCl in 1,4-dioxane (30.5 mL, 122 mmol). After stirring for 3 days at RT, the mixture was filtered, rinsing with 1,4-dioxane. The filter cake was dried in a vacuum oven at 50° C. to obtain the title compound (3.49 g, 88%) as a white solid. ES-MS (m/z, ⁷⁹Br/⁸¹Br) 288.0/290.0 (M+H).

### Preparation 410

### tert-Butyl (R)-(1-((1-(5-bromo-3-(trifluoromethyl)-1H-pyrazol-1-yl)-2-methylpropan-2-yl-1,1-d2)amino)-3-(1-(methyl-d3)-1H-pyrazol-4-yl)-1-oxopropan-2-yl)carbamate

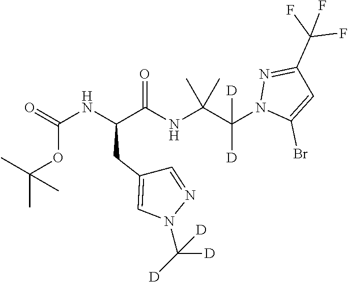 [SMILES: [2H]C([2H])([2H])n1cc(C[C@@H](NC(=O)OC(C)(C)C)C(=O)NC(C)(C)C([2H])([2H])n2nc(C(F)(F)F)cc2Br)cn1]

To a solution of (R)-2-((tert-butoxycarbonyl)amino)-3-(1-(methyl-d3)-1H-pyrazol-4-yl)propanoic acid (760 mg, 2.79 mmol) in DMF (50 mL) at ambient temperature under nitrogen, was added HATU (1.11 g, 2.93 mmol). After stirring for 10 min, 1-(5-bromo-3-(trifluoromethyl)-1H-pyrazol-1-yl)-2-methylpropan-1,1-d2-2-amine hydrochloride (951 mg, 2.93 mmol) was added followed by DIPEA (972 μL, 5.58 mmol) in 2 portions, keeping the internal reaction temperature below 30° C. The resulting mixture was stirred overnight at ambient temperature, then diluted with EtOAc (50 mL) and washed with 1N aq HCl (3×20 mL) followed by saturated aq NaHCO₃ (3×20 mL). The organic layer was dried over MgSO₄ and concentrated under reduced pressure to provide the title compound (1.41 g, 93%) as a brown foam. ES-MS (m/z, ⁷⁹Br/⁸¹Br) 486.2/488.2 (M-tBu+H).

### Preparation 411

### 5-Bromo-3-fluoro-1-(methyl-d3)pyridin-2(1H)-one

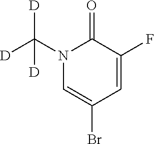 [SMILES: [2H]C([2H])([2H])n1cc(Br)cc(F)c1=O]

To a three-necked RBF was added 5-bromo-3-fluoro-1,2-dihydropyridin-2-one (10.0 g, 52.1 mmol) and potassium carbonate (14.4 g, 104 mmol) in DMF (250 mL). Iodomethane-d3 (3.57 mL, 57.3 mmol) was added dropwise. The reaction was allowed to stir at RT for 1 h. The reaction was quenched with water (500 mL) and extracted with EtOAc (3×250 mL). The combined organic layer was dried over anhydrous MgSO₄, filtered and concentrated under reduced pressure to afford the title compound (6.48 g, 58%) as a tan solid. ES/MS m/z 209.2/211.2 (M+H)

### Preparation 412

### 3-Fluoro-1-(methyl-d3)-5-(4,4,5,5-tetramethyl-1,3,2-dioxaborolan-2-yl)pyridin-2(1H)-one

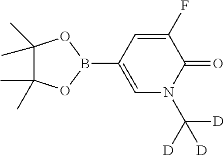 [SMILES: [2H]C([2H])([2H])n1cc(B2OC(C)(C)C(C)(C)O2)cc(F)c1=O]

To a RBF was added: 5-bromo-3-fluoro-1-(methyl-d3)pyridin-2(1H)-one (6.48 g, 31.0 mmol), bis(pinacolato)diboron (15.7 g, 62.0 mmol), potassium acetate (9.13 g, 93.0 mmol) and [1,1′-bis(diphenylphosphino)ferrocene]dichloropalladium(II) complex with DCM (2.53 g, 3.10 mmol) followed by 1,4-Dioxane (200 mL). The mixture was then heated to 100° C. under nitrogen atmosphere overnight. The reaction was cooled to RT and concentrated under reduced pressure. The residue was dissolved in EtOAc (500 mL) and dried over anhydrous MgSO₄. The mixture was filtered over a plug of silica gel, rinsing with EtOAc. The filtrate was evaporated under reduced pressure to afford the title compound (14.8 g, 99+%). ES/MS m/z 257.4 (M+H)

### Preparation 413

### tert-Butyl (R)-(1-((1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)amino)-3-(1-(methyl-d3)-1H-pyrazol-4-yl)-1-oxopropan-2-yl)carbamate

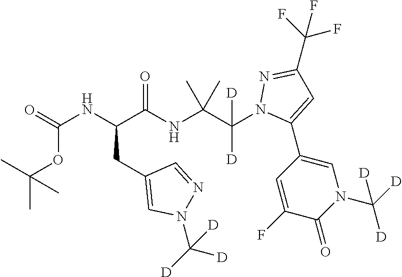 [SMILES: [2H]C([2H])([2H])n1cc(C[C@@H](NC(=O)OC(C)(C)C)C(=O)NC(C)(C)C([2H])([2H])n2nc(C(F)(F)F)cc2-c2cc(F)c(=O)n(C([2H])([2H])[2H])c2)cn1]

To a RBF was added 3-fluoro-1-(methyl-d3)-5-(4,4,5,5-tetramethyl-1,3,2-dioxaborolan-2-yl)pyridin-2(1H)-one (1.33 g, 2 Eq, 5.20 mmol), tert-butyl (R)-(1-((1-(5-bromo-3-(trifluoromethyl)-1H-pyrazol-1-yl)-2-methylpropan-2-yl-1,1-d2)amino)-3-(1-(methyl-d3)-1H-pyrazol-4-yl)-1-oxopropan-2-yl)carbamate (1.41 g, 2.60 mmol), Pd (dppf)-Cl₂ adduct (212 mg, 260 μmol), and potassium carbonate (1.08 g, 7.80 mmol) followed by 1,4-dioxane (9.75 mL) and water (3.25 mL). The reaction mixture was then heated to 90° C. overnight. The reaction was cooled to ambient temperature, diluted with water (100 mL) and extracted with EtOAc (3×50 mL). The combined organic layer was dried over anhydrous MgSO₄, filtered and concentrated under reduced pressure. This residue was purified by column chromatography (SiO₂, 0-5% MeOH in EtOAc gradient) to give the title compound (740 mg, 48%) as an orange-brown foam. ES/MS m/z 592.3 (M+H)

### Preparation 414

### (R)-2-Amino-N-(1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)-3-(1-(methyl-d3)-1H-pyrazol-4-yl)propanamide hydrochloride

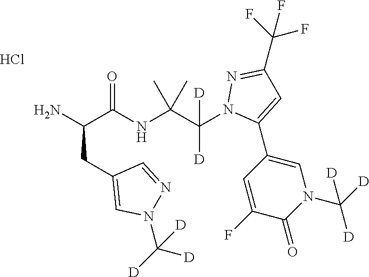 [SMILES: Cl ; [2H]C([2H])([2H])n1cc(C[C@@H](N)C(=O)NC(C)(C)C([2H])([2H])n2nc(C(F)(F)F)cc2-c2cc(F)c(=O)n(C([2H])([2H])[2H])c2)cn1]

tert-Butyl (R)-(1-((1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)amino)-3-(1-(methyl-d3)-1H-pyrazol-4-yl)-1-oxopropan-2-yl)carbamate (740 mg, 1.25 mmol) was dissolved in 1,4-dioxane (25 mL) under nitrogen atmosphere. Then, hydrogen chloride in dioxane (1.56 mL, 4 M, 6.25 mmol) was added dropwise. The reaction was allowed to stir at RT until complete conversion at which time it was concentrated under reduced pressure. The residue was dissolved in MeOH (10 mL) and DCM (20 mL) and concentrated under reduced pressure to afford the title compound (600 mg, 79%) as a yellow solid. ES/MS m/z 492.2 (M+H)

### Preparation 415

### 2-Bromo-1,5-difluoro-3-vinylbenzene

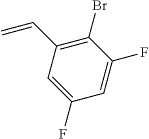

A mixture of methyltriphenylphosphonium bromide (9.70 g, 27.1 mmol) in THF (80 mL) was evacuated and backfilled with N₂×3, cooled to 0° C., and 1.0 M t-BuOK in THF (29.4 mL, 29.4 mmol) was added dropwise. The reaction mixture was stirred at 0° C. for 30 min, then a solution of 2-bromo-3,5-difluorobenzaldehyde (5.00 g, 22.6 mmol) in THF (40 mL) was added at 0° C. The reaction mixture was then stirred at 27° C. for 2 h, diluted with H₂O (100 mL) and extracted with EtOAc (100 mL×2). The combined organics were washed with saturated aqueous NaCl (100 mL×2), dried over anhydrous Na₂SO₄, filtered, and concentrated under reduced pressure. Flash chromatography (SiO₂, eluted with hexanes) provided the title compound (1.9 g, 36%) as a colorless oil.

### Preparation 416

### 2-(2-Bromo-3,5-difluorophenyl)ethan-1-ol

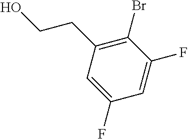 [SMILES: OCCc1cc(F)cc(F)c1Br]

A solution of 2-bromo-1,5-difluoro-3-vinylbenzene (1.90 g, 8.24 mmol) in THF (20 mL) was evacuated and backfilled with N₂ three times and cooled to 0° C. Then BH₃·SMe₂ (1.65 mL, 16.5 mmol) was added at 0° C. The reaction mixture was stirred at 27° C. for 16 h, cooled to 0° C., and 2N aq. NaOH (16.5 mL, 33.0 mmol) and 30% aq. H₂O₂ (3.37 mL, 33.0 mmol) were added dropwise at 0° C. The reaction mixture was stirred at 27° C. for 16 h, quenched with sat. aq. Na₂SO₃ (50 mL) and extracted with EtOAc (50 mL×2). The organics were washed with sat. aq. Na₂SO₃ (50 mL×2), dried over Na₂SO₄, filtered, and concentrated under reduced pressure. Flash chromatography (SiO₂, gradient 0-20% EtOAc in hexanes) provided 1-(2-bromo-3,5-difluorophenyl)ethan-1-ol (450 mg, 21%) as a white solid, followed by the title compound (740 mg, 34%) as a colorless oil. 1H NMR (CDCl₃) δ 6.93-6.87 (m, 1H), 6.84-6.77 (m, 1H), 3.91 (t, J=6.8 Hz, 2H), 3.06 (t, J=6.8 Hz, 2H).

### Preparation 417

### 2-Bromo-1,5-difluoro-3-(2-((2-methoxyethoxy)methoxy)ethyl)benzene

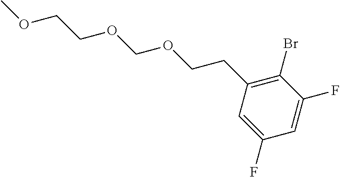 [SMILES: COCCOCOCCc1cc(F)cc(F)c1Br]

A mixture of 2-(2-bromo-3,5-difluorophenyl)ethan-1-ol (740 mg, 2.81 mmol) and DIEA (1.96 mL, 11.2 mmol) in CH₂Cl₂ (10 mL) was evacuated and backfilled with N₂×3, cooled to 0° C., and treated dropwise with 1-(chloromethoxy)-2-methoxyethane (641 μL, 5.62 mmol). The reaction mixture was stirred at 26° C. for 16 h, diluted with H₂O (10 mL) and extracted with DCM (10 mL×2). The combined organics were washed with saturated aqueous NaCl (10 mL×2), dried over Na₂SO₄, filtered, and concentrated under reduced pressure. Flash chromatography (SiO₂, 0-8% EtOAc in hexanes) provided the title compound (780 mg, 81%) as a colorless oil. 1H NMR (CDCl₃) δ 6.94-6.87 (m, 1H), 6.82-6.75 (m, 1H), 4.71 (s, 2H), 3.81 (t, J=6.8 Hz, 2H), 3.66-3.61 (m, 2H), 3.55-3.51 (m, 2H), 3.39 (s, 3H), 3.07 (t, J=6.8 Hz, 2H).

### Preparation 418

### 5-Bromo-6,8-difluoroisochromane

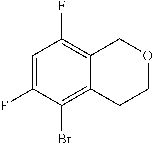 [SMILES: Fc1cc(F)c2c(c1Br)CCOC2]

A solution of 2-bromo-1,5-difluoro-3-(2-((2methoxyethoxy)methoxy)ethyl)benzene (680 mg, 1.99 mmol) in CH₂Cl₂ (10 mL) was evacuated and backfilled with N₂×3, cooled to 0° C., treated dropwise with 1.0 M TiCl₄ in CH₂Cl₂ (3.97 mL, 3.97 mmol), and stirred at 26° C. for 16 h, filtered, and concentrated under reduced pressure. Flash chromatography (SiO₂, gradient 0-4% EtOAc in hexanes) provided the title compound (430 mg, 83%) as a white solid. 1H NMR (DMSO-d6) δ 7.36 (t, J=9.6 Hz, 1H), 4.67 (s, 2H), 3.91 (t, J=5.6 Hz, 2H), 2.72 (t, J=5.6 Hz, 2H).

### Preparation 419

### 5-Bromo-6,8-difluoroisochroman-1-one

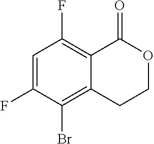 [SMILES: O=C1OCCc2c(Br)c(F)cc(F)c21]

To a mixture of 5-bromo-6,8-difluoroisochromane (460 mg, 1.75 mmol) and CuCl (86.9 mg, 877 μmol) in t-BuOH (6 mL) was added 6.0 M tert-butyl hydroperoxide in decane (1.17 mL, 7.02 mmol) at 26° C. The reaction mixture was evacuated and backfilled with N₂×3, then stirred at 60° C. for 16 h. The reaction mixture was treated with concentrated aq. NH₃ (0.5 mL), poured into H₂O (10 mL) and extracted with EtOAc (10 mL×3). The combined organics were washed with saturated aqueous NaCl (20 mL), dried over Na₂SO₄, filtered, and concentrated in vacuo. Flash chromatography (SiO₂, gradient 0-30% EtOAc in hexanes) provided the title compound (180 mg, 37%) as a white solid. ES-MS m/z 262.8/264.8 (M+H) (Br^{79/81}).

### Preparation 420

### (R)-3-Bromo-2-(2-chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-4,6-difluorobenzamide

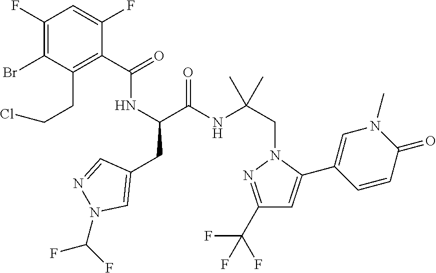 [SMILES: Cn1cc(-c2cc(C(F)(F)F)nn2CC(C)(C)NC(=O)[C@@H](Cc2cnn(C(F)F)c2)NC(=O)c2c(F)cc(F)c(Br)c2CCCl)ccc1=O]

To a mixture of 5-bromo-6,8-difluoroisochroman-1-one (180 mg, 95% Wt, 650 μmol) in SOCl₂ (2.37 mL, 32.5 mmol) was added DMF (75.5 μL, 975 μmol). The reaction mixture was degassed and purged with N₂ 3×, then the reaction was heated to 100° C. and stirred under N₂ for 16 h. The reaction was then allowed to cool to RT and concentrated under reduced pressure to afford the corresponding acyl chloride as a yellow oil, which was then re-dissolved in THF (3 mL) and carried forward crude assuming quantitative yield and mass recovery. In a separate container, a mixture of (R)-2-amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide hydrochloride (290 mg, 90% wt, 485 μmol) and TEA (676 μL, 4.85 mmol) in THF (3 mL) was prepared. This solution was cooled to 0° C., then the acyl chloride solution was added. The cooling bath was removed, and the mixture was stirred at 26° C. for 1 hour, after which the reaction was diluted with water (20 mL) and extracted with EtOAc (2×20 mL). The combined organic layers were washed with saturated aqueous NaCl (20 mL), dried over Na₂SO₄, filtered, concentrated under reduced pressure, and purified by silica gel chromatography using a gradient of 0-100% EtOAc in hexanes to afford the title compound (250 mg, 63%) as a white solid. ES-MS m/z 781.9, 783.8, 785.8 (M+H).

### Preparation 421

### (R)-2-(5-Bromo-6,8-difluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

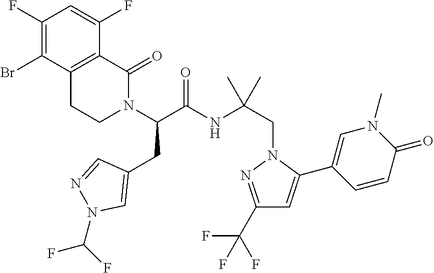 [SMILES: Cn1cc(-c2cc(C(F)(F)F)nn2CC(C)(C)NC(=O)[C@@H](Cc2cnn(C(F)F)c2)N2CCc3c(Br)c(F)cc(F)c3C2=O)ccc1=O]

To a solution of (R)-3-bromo-2-(2-chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-4,6-difluorobenzamide (250 mg, 96% wt, 307 μmol) in ACN (5 mL) was added Cs₂CO₃ (150 mg, 460 μmol). The reaction mixture was stirred at 26° C. for 1 h, after which it was diluted with water (20 mL) and extracted with EtOAc (2×20 mL). The combined organic layers were washed with saturated aqueous NaCl (20 mL), dried over Na₂SO₄, filtered, concentrated under reduced pressure, and purified by silica gel chromatography using a gradient of 0-100% EtOAc in hexanes to afford the title compound (190 mg, 80%) as a white solid. ES-MS m/z 745.8, 747.7 (M+H).

### Preparation 422

### (R)-2-(6,8-Difluoro-1-oxo-5-(4,4,5,5-tetramethyl-1,3,2-dioxaborolan-2-yl)-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propenamide

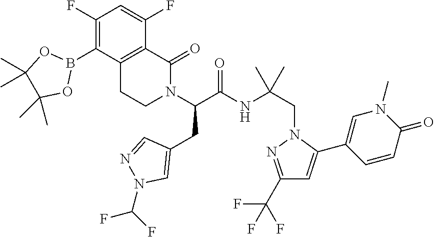 [SMILES: Cn1cc(-c2cc(C(F)(F)F)nn2CC(C)(C)NC(=O)[C@@H](Cc2cnn(C(F)F)c2)N2CCc3c(B4OC(C)(C)C(C)(C)O4)c(F)cc(F)c3C2=O)ccc1=O]

In a nitrogen-filled glovebox, a mixture of (R)-2-(5-bromo-6,8-difluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (70 mg, 96% wt, 90 μmol), 4,4,4′,4′,5,5,5′,5′-octamethyl-2,2′-bi (1,3,2-dioxaborolane) (0.11 g, 0.45 mmol) and PCy3 Pd G4 (6.0 mg, 9.0 μmol) in toluene (1.5 mL) was treated with potassium acetate (27 mg, 0.27 mmol). The reaction vial was sealed, taken out from the glovebox, and stirred at 100° C. for 3 h. The reaction mixture was cooled to RT, then the mixture was filtered and the filtrate concentrated under reduced pressure. The residue was purified by column chromatography (SiO2, 0-100% EtOAc in heptane gradient) to give the title compound (70 mg, 68%) as a gray solid. ES/MS m/z 793.9 (M+H).

### Preparation 423

### Ethyl 3-bromo-6-fluoro-2-methylbenzoate

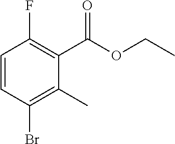

In a 100-mL flask under nitrogen, 3-bromo-6-fluoro-2-methylbenzoic acid (3.00 g, 12.9 mmol) and potassium carbonate (4.98 g, 36.0 mmol) were suspended in DMF (18.4 mL). The mixture was treated with iodoethane (0.885 mL, 12.9 mmol) and the reaction mixture was stirred at RT. After 23 h, the reaction mixture was diluted with EtOAc (50 mL) and water (50 mL). The layers were separated, and the aqueous layer was extracted with EtOAc (30 mL×2). The combined organic layers were washed with saturated aqueous NaCl (15 mL×2), dried over sodium sulfate, and filtered. The filtrate was concentrated under a stream of air to provide the title compound (2.57 g, 77%) as a pale-yellow oil. ES/MS m/z 261.0/263.0 (M+H), Br isotope.

### Preparation 424

### Ethyl 3-bromo-2-(bromomethyl)-6-fluorobenzoate

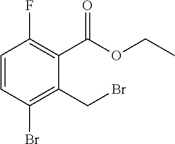

In a 100-mL flask under nitrogen, ethyl 3-bromo-6-fluoro-2-methylbenzoate (1.50 g, 5.75 mmol) was dissolved in anhydrous 1,2-DCE (28.7 mL). The solution was treated with NBS (1.12 g, 6.29 mmol) then AIBN (94 mg, 0.57 mmol) at RT. The headspace of the flask was purged with nitrogen for an additional minute, then the mixture was heated to 85° C. (heating block temperature) under a positive pressure of nitrogen. After 45 h, the reaction mixture was cooled to RT then quenched by addition of saturated aqueous sodium bicarbonate solution (20 mL). The layers were separated, and the organic layer was dried over sodium sulfate. The dried solution was filtered and the filtrate concentrated under reduced pressure to provide the title compound (1.81 g, 93%) as a light brown oil. No ionization.

### Preparation 425

### 4-Bromo-7-fluoro-3-hydroxyisobenzofuran-1 (3H)-one

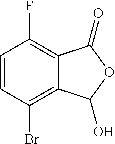 [SMILES: O=C1OC(O)c2c(Br)ccc(F)c21]

In a 40-mL vial, ethyl 3-bromo-2-(bromomethyl)-6-fluorobenzoate (1.00 g, 2.94 mmol) was dissolved in DCM (5.88 mL) then was treated with N-methylmorpholine N-oxide (517 mg, 4.41 mmol). The resulting mixture was stirred at RT for 43 h. The reaction mixture was partitioned between DCM (15 mL) and aqueous 0.7 M NaH₂PO₄ solution (15 mL). The layers were separated, and the organic layer was washed with saturated aqueous NaCl (10 mL). The washed solution was dried over sodium sulfate, filtered, and the filtrate concentrated under reduced pressure. The residue was purified by column chromatography (SiO2, 0-30% EtOAc in heptane gradient) to give the title compound (72 mg, 10%) as a white solid. ES/MS m/z 264.0/266.0 (M+NH₄), Br isotope.

### Preparation 426

### (R)-2-(5-Bromo-8-fluoro-1-oxophthalazin-2(1H)-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide

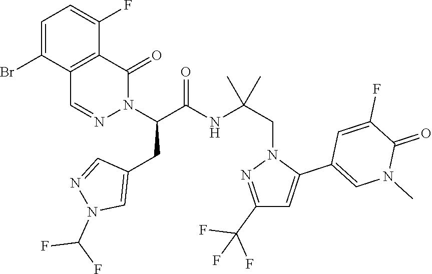 [SMILES: Cn1cc(-c2cc(C(F)(F)F)nn2CC(C)(C)NC(=O)[C@@H](Cc2cnn(C(F)F)c2)n2ncc3c(Br)ccc(F)c3c2=O)cc(F)c1=O]

In a 1-dram vial, (R)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-hydrazineylpropanamide (60 mg, 0.11 mmol) and 4-bromo-7-fluoro-3-hydroxyisobenzofuran-1 (3H)-one (28 mg, 0.11 mmol) were dissolved in EtOH (0.56 mL). The mixture was heated to 90° C. for 2 h (heating block temperature), then the mixture was allowed to stir at RT for an additional 15 h. The reaction mixture was concentrated under a stream of nitrogen. The crude material was combined with the crude material of an identical reaction run on 30 mg of the hydrazine starting material. The combined residue was purified by column chromatography (SiO2, 10-50% 3:1 EtOAc/EtOH in heptane gradient) to give the title compound (75 mg, 59% combined yield) as a white solid. ES/MS m/z 745.2/747.2 (M+H), Br isotope.

### Preparation 427

### Methyl 3-fluoro-5-(2-((tetrahydro-2H-pyran-2-yl)oxy)ethyl)isonicotinate

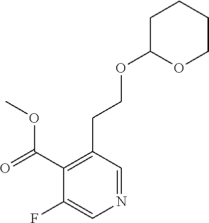 [SMILES: COC(=O)c1c(F)cncc1CCOC1CCCCO1]

To a mixture of methyl 3-bromo-5-fluoroisonicotinate (5.00 g, 21.4 mmol) and potassium trifluoro (2-((tetrahydro-2H-pyran-2-yl)oxy)ethyl)borate (15.3 g, 64.8 mmol) in 1,4-dioxane (100 mL) and water (10 mL) were added cesium carbonate (21.0 g, 64.5 mmol) and cataCXium® A Pd G3 (4.67 g, 6.41 mmol). The reaction mixture was degassed and purged with nitrogen (3×), then the reaction mixture was stirred at 110° C. for 3 h under nitrogen. The reaction mixture was cooled to RT then filtered. The filtrate was diluted with water (100 mL) and extracted with EtOAc (100 mL×3). The combined organic layers were dried over anhydrous Na₂SO₄, filtered, and the filtrate concentrated under reduced pressure. The residue was purified by column chromatography (SiO2, 0-30% EtOAc in heptane gradient) to give the title compound (2.6 g, 39%) as a brown oil. ES/MS m/z 284.0 (M+H)

### Preparation 428

### 8-Fluoro-3,4-dihydro-1H-pyrano[4,3-c]pyridin-1-one

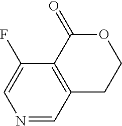 [SMILES: O=C1OCCc2cncc(F)c21]

To a mixture of methyl 3-fluoro-5-(2-((tetrahydro-2H-pyran-2-yl)oxy)ethyl)isonicotinate (2.6 g, 8.3 mmol) in THF (150 mL) was added hydrogen chloride (2.0 in water (31 mL, 62 mmol). The reaction mixture was stirred at ambient temperature for 12 h. Then, the pH of the mixture was adjusted to ˜7 with saturated aqueous NaHCO₃. The mixture was diluted with H₂O (50 mL) and extracted with EtOAc (100 mL×3). The combined organic layers were washed with saturated aqueous NaCl (100 mL), dried over anhydrous Na₂SO₄, filtered and concentrated under reduced pressure. The crude residue was purified by flash column chromatography (SiO₂, 47-49% THF/hexanes gradient) to afford the title compound (920 mg, 61%) as a white solid. ES/MS m/z 168.1 (M+H)

### Preparation 429

### 8-Fluoro-1-oxo-3,4-dihydro-1H-pyrano[4,3-c]pyridine 6-oxide

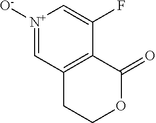 [SMILES: O=C1OCCc2c[n+]([O-])cc(F)c21]

To a mixture of 8-fluoro-3,4-dihydro-1H-pyrano[4,3-c]pyridin-1-one (450 mg, 2.45 mmol) in DCM (20 mL) was added 3-chlorobenzoperoxoic acid (998 mg, 4.92 mmol) in portions at 0° C. The reaction mixture was stirred at ambient temperature for 16 h. The reaction was quenched with NaHSO₃ solution (800 mg in 20 mL water), then the reaction mixture was stirred at ambient temperature for 30 min. The mixture was diluted with H₂O (30 mL) and extracted with DCM (30 mL×6). The combined organic layers were dried over anhydrous Na₂SO₄, filtered and concentrated under reduced pressure. The residue was purified by column chromatography (SiO₂, 75-100% THF/hexanes gradient) to afford the title compound (380 mg, 76%) as a white solid. ES/MS m/z 184.0 (M+H)

### Preparation 430

### 5-Chloro-8-fluoro-3,4-dihydro-1H-pyrano[4,3-c]pyridin-1-one

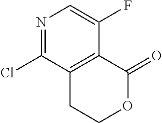 [SMILES: O=C1OCCc2c(Cl)ncc(F)c21]

To a mixture of 8-fluoro-1-oxo-3,4-dihydro-1H-pyrano[4,3-c]pyridine 6-oxide (380 mg, 90% wt, 1.87 mmol) in DCE (20 mL) was added phosphorus oxychloride (3.0 mL, 33 mmol) at 22° C. Then the reaction mixture was stirred at 85° C. for 5 h. The reaction mixture was cooled to 22° C., then was added to water (40 mL) slowly, the mixture was stirred at 22° C. for 30 min. The pH of the mixture was adjusted to ˜7 with saturated NaHCO₃ solution, then extracted with DCM (50 mL×3). The combined organic layers were dried over anhydrous Na₂SO₄, filtered and concentrated under reduced pressure. The residue was purified by column chromatography (SiO₂, 16˜19% THF/hexanes gradient) to afford the title compound (200 mg, 47%) as a white solid. ES/MS m/z 201.9/203.8 (M+H)

### Preparation 431

### (R)-2-Chloro-3-(2-chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-5-fluoroisonicotinamide

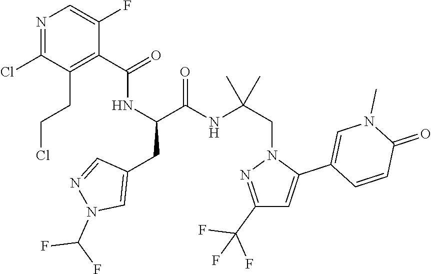 [SMILES: Cn1cc(-c2cc(C(F)(F)F)nn2CC(C)(C)NC(=O)[C@@H](Cc2cnn(C(F)F)c2)NC(=O)c2c(F)cnc(Cl)c2CCCl)ccc1=O]

To a mixture of 5-chloro-8-fluoro-3,4-dihydro-1H-pyrano[4,3-c]pyridin-1-one (200 mg, 88% wt, 873 μmol) and benzyl(triethyl)ammonium chloride (400 mg, 1.76 mmol) in DCE (2 mL) were added thionyl chloride (360 μL, 4.89 mmol) and boron trifluoride etherate (260 μL, 2.03 mmol) at 25° C. . . . Then the reaction mixture was gradually heated up to 110° C. and stirred at 110° C. for 48 h. The reaction mixture was cooled to 22° C., concentrated under reduced pressure. Then (R)-2-amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide hydrochloride (270 mg, 90% wt, 452 μmol) in DMA (3 mL) and TEA (700 μL, 5.02 mmol) was added at 25° C. After stirring for 30 min, HATU (251 mg, 660 μmol) was added to the mixture. Then the mixture was stirred at 25° C. for 8 h. The reaction mixture was diluted with H₂O (20 mL) and extracted with EtOAc (20 mL×3). The combined organic layers were washed with saturated aqueous NaCl (20 mL×3), dried over anhydrous Na₂SO₄, filtered and concentrated under reduced pressure. The residue was purified by column chromatography (SiO₂, 0˜100% EtOAc/hexanes) to afford the title compound (94 mg, 25%) as a yellow oil. ES/MS m/z 721.1, 722.9, 725.3 (M+H)

### Preparation 432

### (R)-2-(5-Chloro-8-fluoro-1-oxo-3,4-dihydro-2,6-naphthyridin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

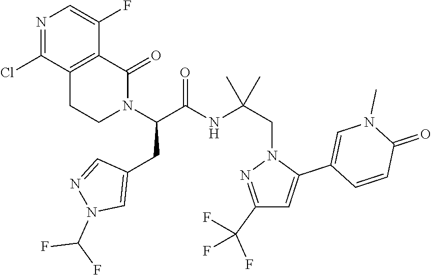 [SMILES: Cn1cc(-c2cc(C(F)(F)F)nn2CC(C)(C)NC(=O)[C@@H](Cc2cnn(C(F)F)c2)N2CCc3c(Cl)ncc(F)c3C2=O)ccc1=O]

To a solution of (R)-2-chloro-3-(2-chloroethyl)-N-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-5-fluoroisonicotinamide (94 mg, 0.11 mmol) in ACN (1.5 mL) was added Cs₂CO₃ (55 mg, 0.17 mmol). Then the reaction mixture was stirred at 20° C. for 16 h. The reaction mixture was diluted with H₂O (10 mL) and extracted with EtOAc (10 mL×2). The combined organic layers were washed with saturated aqueous NaCl (10 mL×2), dried over anhydrous Na₂SO₄, filtered and concentrated under reduced pressure to give a crude product. The crude product was purified by prep-TLC (DCM:MeOH=10:1, Rf=0.5). The title compound (64 mg, 77% yield) was obtained as a yellow solid. ES/MS m/z 685.2, 687.1 (M+H).

### Preparation 433

### (R)-3-Bromo-2-(2-chloroethyl)-6-fluoro-N-(1-((1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)amino)-3-(1-(methyl-d3)-1H-pyrazol-4-yl)-1-oxopropan-2-yl)benzamide

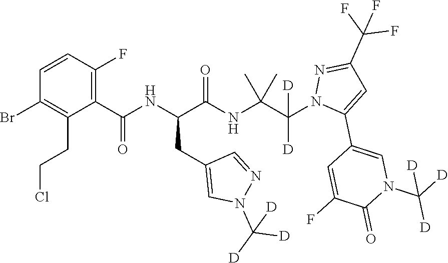 [SMILES: [2H]C([2H])([2H])n1cc(C[C@@H](NC(=O)c2c(F)ccc(Br)c2CCCl)C(=O)NC(C)(C)C([2H])([2H])n2nc(C(F)(F)F)cc2-c2cc(F)c(=O)n(C([2H])([2H])[2H])c2)cn1]

To a solution of (R)-2-amino-N-(1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)-3-(1-(methyl-d3)-1H-pyrazol-4-yl)propanamide hydrochloride (118.9 mg, 225.2 μmol) in DCM (1 mL) was added DIEA (116.4 mg, 155 μL, 900.8 μmol) and cooled to 0° C. To this solution was added 3-bromo-2-(2-chloroethyl)-6-fluorobenzoyl chloride (67.55 mg, 225.2 μmol). After stirring for 30 min at 22° C., the mixture was concentrated under reduced pressure then diluted with EtOAc (150 mL) and washed with water, 1N HCl, saturated NaHCO₃ and saturated aqueous NaCl. The organic phase was dried over magnesium sulfate and concentrated under reduced pressure to yield the title compound (170 mg, 99+% yield) as a light tan foam solid. ES/MS m/z 756.2 (M+H).

### Preparation 434

### (R)-2-(5-Bromo-8-fluoro-1-oxo-3,4-dihydroisoquinolin-2 (1H)-yl)-N-(1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)-3-(1-(methyl-d3)-1H-pyrazol-4-yl)propanamide

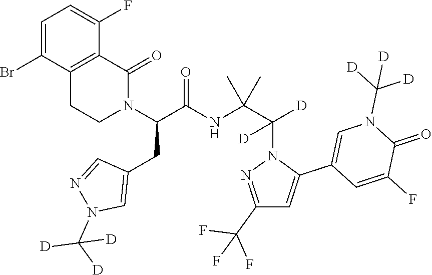 [SMILES: [2H]C([2H])([2H])n1cc(C[C@H](C(=O)NC(C)(C)C([2H])([2H])n2nc(C(F)(F)F)cc2-c2cc(F)c(=O)n(C([2H])([2H])[2H])c2)N2CCc3c(Br)ccc(F)c3C2=O)cn1]

To a mixture of (R)-3-bromo-2-(2-chloroethyl)-6-fluoro-N-(1-((1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)amino)-3-(1-(methyl-d3)-1H-pyrazol-4-yl)-1-oxopropan-2-yl)benzamide (170.0 mg, 225.2 μmol) in ACN (2 mL) was added cesium carbonate (293 mg, 900 μmol). The mixture was stirred at 45° C. for 2 h, then cooled to ambient temperature, diluted with EtOAc and washed with 1N HCl and saturated NaHCO₃, then dried, filtered and concentrated under reduced pressure. The residue was purified by column chromatography (SiO₂, 0-5% MeOH in DCM) to afford the title compound (119.4 mg, 68%) as a white solid. ES/MS m/z 718.2 (M+H).

### Preparation 435

### 5-Chlorothiazolo[5,4-b]pyridine-2-thiol

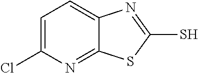 [SMILES: Sc1nc2ccc(Cl)nc2s1]

A mixture of 2,6-dichloro-pyridin-3-ylamine (1.01 g, 6.20 mmol) and potassium ethyl xanthate (1.98 g, 12.4 mmol) in DMF (7 mL) was gradually heated up to 130° C. and stirred for 16 h. The reaction was allowed to cool to RT and water (10 mL) and 1 M aq HCl (40 mL) were then added, precipitating a solid. The suspension was stirred for an additional 30 min and filtered. The resulting cake was washed with water, triturated with EtOAc (50 mL), filtered to remove solids, and the filtrate concentrated under reduced pressure to yield an orange solid. The resulting orange solid was purified by flash silica gel chromatography, eluting with 0˜60% EtOAc/hexanes to afford the title compound (1.1 g, 81%) as a yellow solid. ES-MS m/z 202.9, 204.9 (M+H).

### Preparation 436

### 5-Chloro-2-(methylthio) thiazolo[5,4-b]pyridine

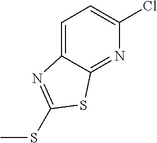

A mixture of 5-chlorothiazolo[5,4-b]pyridine-2-thiol (1.1 g, 5.0 mmol) in dry THF (15 mL) was cooled to 0° C. and sodium hydride in mineral oil (213 mg, 60% wt, 5.32 mmol) was added portion-wise over 10 min. The reaction mixture was stirred for an additional 30 min, then methyl iodide (768 mg, 350 μL, 1.1 Eq, 5.41 mmol) was added dropwise. The cooling bath was removed, and the solution was stirred for 4 h at 20° C. The mixture was quenched with sat. aq. NH₄Cl (20 mL), and the resulting mixture was extracted with EtOAc (2×15 mL), washed with saturated aqueous NaCl (10 mL), dried over Na₂SO₄, filtered and concentrated under reduced pressure. The crude material was purified on silica, eluting with 0-20% EtOAc/hexanes to afford the title compound (765 mg, 67%) as a light-yellow solid. ES-MS m/z 216.8, 218.8 (M+H).

### Preparation 437

### 5-Chloro-2-methoxythiazolo[5,4-b]pyridine

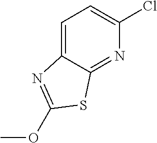

To a solution of 5-chloro-2-(methylthio) thiazolo[5,4-b]pyridine (761 mg, 3.34 mmol) in DCM (10 mL) and MeOH (10 mL) was added sodium methoxide (539 mg, 9.98 mmol) and the resulting mixture was stirred at 20° C. for 16 h. The reaction was quenched with sat. aq. NH₄Cl (30 mL), and the resulting mixture was extracted with EtOAc (2×25 mL), washed with saturated aqueous NaCl (30 mL), dried over Na₂SO₄, filtered, and concentrated under reduced pressure. The crude material was purified on silica, eluting with 0-10% EtOAc/hexanes to afford the title compound (533 mg, 64%) as a white solid. ES-MS m/z 201.0, 202.9 (M+H).

### Preparation 438

### 5-Chlorothiazolo[5,4-b]pyridin-2(1H)-one

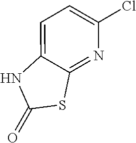 [SMILES: O=c1[nH]c2ccc(Cl)nc2s1]

To a solution of 5-chloro-2-methoxythiazolo[5,4-b]pyridine (533.0 mg, 80% wt, 1 Eq, 2.125 mmol) in 1,4-dioxane (10 mL) was added 12 M HCl (2.00 mL, 24.0 mmol) at 20° C. The resulting mixture was gradually heated to 60° C. and stirred for 4 h. The reaction mixture was concentrated under reduced pressure. The crude product was triturated with DCM (15 mL) at 20° C. for 2 h. The mixture was filtered to give a cake, then the filter cake was dried under reduced pressure to afford the title compound (270 mg, 56%) as a white solid. ES-MS m/z 186.9, 188.9 (M+H)

### Preparation 439

### 5-Chloro-1-methylthiazolo[5,4-b]pyridin-2(1H)-one

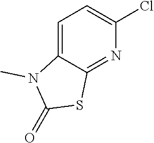

To a solution of 5-chlorothiazolo[5,4-b]pyridin-2(1H)-one (113 mg, 558 μmol) in AcOH (10 mL) was added 1-bromopyrrolidine-2,5-dione (1.2 g, 6.7 mmol) at 20° C. The reaction mixture was stirred at 110° C. for 48 h, then was cooled and concentrated under reduced pressure. The resulting residue was then diluted with EtOAc (20 mL), washed with water (10 mL×2) and saturated aqueous NaCl (10 mL). The organic phase was dried over anhydrous Na₂SO₄ and concentrated under reduced pressure. The crude product was purified on silica, eluting with 0-20% EtOAc/hexanes to afford the title compound (31 mg, 18%) as a light-yellow solid. ES-MS m/z 280.8 (M+H).

### Preparation 440

### 6-Bromo-5-chloro-1-methylthiazolo[5,4-b]pyridin-2(1H)-one

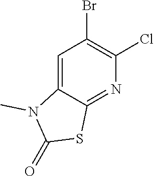

To a solution of 5-chloro-1-methylthiazolo[5,4-b]pyridin-2(1H)-one (300 mg, 1.44 mmol) in AcOH (20 mL) was added NBS (3.03 g, 17.0 mmol) at 20° C. The mixture was stirred at 110° C. for 48 h. Additional NBS (1.51 g, 8.48 mmol) was added into the above mixture. The mixture was stirred at 110° C. for 16 h then, was concentrated under reduced pressure and re-diluted with EtOAc (20 mL). The resulting solution was washed with water (10 mL×2), followed by saturated aqueous NaCl (10 mL). The organic phase was dried over anhydrous Na₂SO₄ and concentrated under reduced pressure. The crude product was purified by flash silica gel chromatography, eluting with 0-20% EtOAc/hexanes to afford the title compound (191 mg, 18%) as a yellow solid. ES-MS m/z 280.8 (M+H).

### Preparation 441

### 5-chloro-1-methyl-2-oxo-1,2-dihydrothiazolo[5,4-b]pyridine-6-carbonitrile

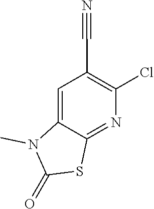

A mixture of 6-bromo-5-chloro-1-methylthiazolo[5,4-b]pyridin-2(1H)-one (300 mg, 0.966 mmol), zinc (II) cyanide (0.550 g, 4.68 mmol) and bis[tris(tert-butyl)phosphine]palladium (99 mg, 0.2 mmol) in DMA (5 mL) was degassed and purged with N₂ (3×), and then the mixture was stirred at 100° C. for 16 h under N₂. The reaction mixture was quenched by aqueous 25% ammonia (5 mL), and the resulting mixture was extracted by EtOAc (30 mL). The organic layer was washed with saturated aqueous NaCl (10 mL×2), dried over anhydrous Na₂SO₄, filtered, and concentrated to give the residue as a light yellow solid. The residue was purified by flash silica gel chromatography (20% THF in hexanes) to give the title compound (47 mg, 18%) as a white solid. ES-MS m/z 225.8, 227.8 (M+H).

### Preparation 442

### tert-Butyl (1-(5-(4-(dimethylphosphoryl)phenyl)-3-(trifluoromethyl)-1H-pyrazol-1-yl)-2-methylpropan-2-yl)carbamate

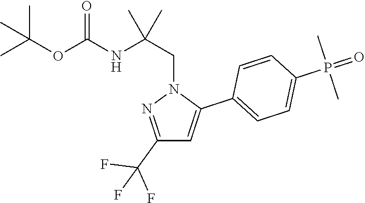 [SMILES: CC(C)(Cn1nc(C(F)(F)F)cc1-c1ccc(P(C)(C)=O)cc1)NC(=O)OC(C)(C)C]

A mixture of (4-bromophenyl)dimethylphosphine oxide (100 mg, 429 μmol), Na₂CO₃ (136.4 mg, 1.287 mmol) and tert-butyl (1-(5-(1,3,6,2-dioxazaborocan-2-yl)-3-(trifluoromethyl)-1H-pyrazol-1-yl)-2-methylpropan-2-yl)carbamate (180.5 mg, 429.5 μmol) in 1,4-dioxane (2 mL) and H₂O (0.2 mL) was treated with XPhos Pd G3 (36.1 mg, 42.6 μmol). The mixture was evacuated and backfilled with N₂×3, then stirred at 90° C. for 1 h, filtered, and rinsed with EtOAc (5 mL×3). The filtrate was concentrated under reduced pressure. Flash chromatography (SiO₂, gradient 0-3% MeOH in CH₂Cl₂) provided the title compound (143.3 mg, 69%) as a white solid. ES-MS m/z 460.1 (M+H).

### Preparation 443

### tert-Butyl (1-(5-(4-(diethylphosphoryl)-3-(methylamino)phenyl)-3-(trifluoromethyl)-1H-pyrazol-1-yl)-2-methylpropan-2-yl)carbamate

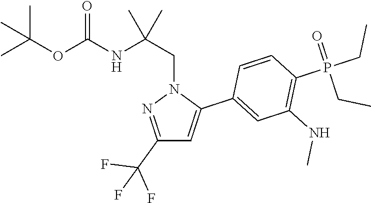 [SMILES: CCP(=O)(CC)c1ccc(-c2cc(C(F)(F)F)nn2CC(C)(C)NC(=O)OC(C)(C)C)cc1NC]

A mixture of tert-butyl (1-(5-(1,3,6,2-dioxazaborocan-2-yl)-3-(trifluoromethyl)-1H-pyrazol-1-yl)-2-methylpropan-2-yl)carbamate (437 mg, 1.04 mmol), 1 N aq. Na₂CO₃ (3.2 mL, 3.2 mmol), (4-bromo-2-(methylamino)phenyl)diethylphosphine oxide (302 mg, 1.04 mmol) and BrettPhos Pd G3 (97 mg, 0.11 mmol) in 1,4-dioxane (7.0 mL) and IPA (2.0 mL) was evacuated and backfilled with N₂×3. The mixture was stirred at 70° C. for 16 h, diluted with water (30 mL), extracted with EtOAc (30 mL×2), washed with saturated aqueous NaCl (30 mL), dried over Na₂SO₄, filtered, and concentrated under reduced pressure. Flash chromatography (SiO₂, gradient 0-3% MeOH in CH₂Cl₂) provided the title compound (474 mg, 86%) as a yellow solid. ES-MS m/z 517.3 (M+H).

### Preparation 444

### tert-Butyl (1-(5-(3-(dimethylphosphoryl)phenyl)-3-(trifluoromethyl)-1H-pyrazol-1-yl)-2-methylpropan-2-yl)carbamate

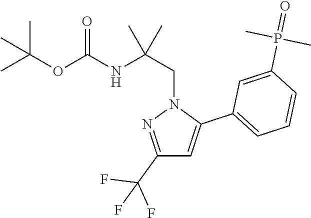 [SMILES: CC(C)(Cn1nc(C(F)(F)F)cc1-c1cccc(P(C)(C)=O)c1)NC(=O)OC(C)(C)C]

The title compound was prepared according to Preparation 442 using (3-bromophenyl)dimethylphosphine oxide as the aryl bromide coupling partner (76% yield). ES-MS m/z 460.1 (M+H).

### Preparation 445

### Benzyl-2-(1-(2-((tert-butoxycarbonyl)amino)-2-methylpropyl)-3-(trifluoromethyl)-1H-pyrazol-5-yl)azetidine-1-carboxylate (Isomer 1)

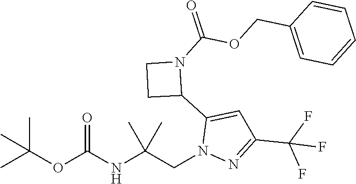 [SMILES: CC(C)(Cn1nc(C(F)(F)F)cc1C1CCN1C(=O)OCc1ccccc1)NC(=O)OC(C)(C)C]

A 40-mL vial was charged with tert-butyl (1-(5-(1,3,6,2-dioxazaborocan-2-yl)-3-(trifluoromethyl)-1H-pyrazol-1-yl)-2-methylpropan-2-yl)carbamate (500 mg, 1.19 mmol) and XPhos Pd G4 (102 mg, 0.118 mmol). A solution of benzyl 3-iodoazetidine-1-carboxylate (755 mg, 2.38 mmol) in 1,4-dioxane (10.0 mL) was added to the vial, followed by a solution of sodium carbonate (441 mg, 4.16 mmol) in water (2.0 mL). The mixture was sparged with nitrogen for 10 min. The nitrogen line was removed, and the mixture was heated to 80° C. for 16 h. The reaction mixture was removed from heat and partitioned between EtOAc (20 mL) and water (20 mL). The organic layer was separated and the aqueous layer was extracted with EtOAc (10 mL). The combined organic layers were concentrated under a stream of nitrogen. The residue was purified by column chromatography (SiO₂, 0-50% MTBE/heptane gradient) followed by chiral SFC (column: Whelk-O RR, 250 mm×30 mm, 5 μm; mobile phase: 95% CO₂-5% MeOH, 40° C., 85 mL/min, first-eluting isomer) to afford the title compound (54 mg, 7.5%) as a colorless glassy solid. ES/MS m/z 397.2 (M+H-Boc).

### Preparation 446

### Benzyl-2-(1-(2-amino-2-methylpropyl)-3-(trifluoromethyl)-1H-pyrazol-5-yl)azetidine-1-carboxylate (Isomer 1)

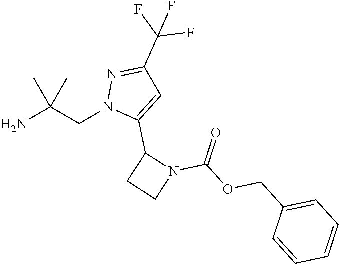 [SMILES: CC(C)(N)Cn1nc(C(F)(F)F)cc1C1CCN1C(=O)OCc1ccccc1]

A solution of benzyl-2-(1-(2-((tert-butoxycarbonyl)amino)-2-methylpropyl)-3-(trifluoromethyl)-1H-pyrazol-5-yl)azetidine-1-carboxylate (Isomer 1) (25 mg, 50 μmol) in DCM (0.50 mL) under argon was cooled to 0° C. in an ice-water bath. 2,6-lutidine (87 μL, 0.75 mmol) was added to the solution, followed by dropwise addition of trimethylsilyl triflate (93 μL, 0.50 mmol). The reaction mixture was stirred at 0° C. for 3 h. The mixture was diluted with DCM (1 mL) and quenched by careful addition of saturated aqueous sodium bicarbonate solution (2 mL). The layers were separated, and the aqueous layer was extracted with DCM (2 mL×2). The combined organic layers were filtered through a plug of sodium sulfate, and the filtrate concentrated under a stream of nitrogen to provide the title compound (22 mg, 99+% yield) as a colorless gum. ES/MS m/z 397.4 (M+H).

### Preparation 447

### tert-Butyl (2-(pyrimidin-2-yl)propan-2-yl)carbamate

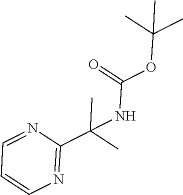 [SMILES: CC(C)(C)OC(=O)NC(C)(C)c1ncccn1]

To a mixture of 2-(pyrimidin-2-yl)propan-2-amine hydrochloride (300 mg, 1.73 mmol) and THF (5 mL) was added Et₃N (485 μL, 3.48 mmol) and di-tert-butyl dicarbonate (440 μL, 1.92 mmol) at 0° C. The mixture was stirred at 20° C. for 2 h, poured into water (10 mL) and extracted with EtOAc (10 mL×3). The combined organics were dried over Na₂SO₄, filtered, and concentrated in vacuo. Flash chromatography (SiO₂, gradient 0-40% EtOAc in hexanes) provided the title compound (341 mg, 80%) as a colorless oil. ES-MS m/z 238.2 (M+H).

### Preparation 448

### tert-Butyl (2-(5-(4,4,5,5-tetramethyl-1,3,2-dioxaborolan-2-yl)pyrimidin-2-yl)propan-2-yl)carbamate

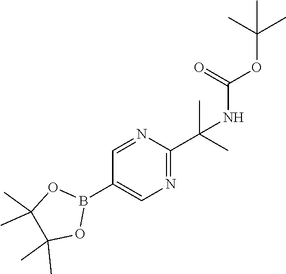 [SMILES: CC(C)(C)OC(=O)NC(C)(C)c1ncc(B2OC(C)(C)C(C)(C)O2)cn1]

In a N₂-filled glovebox, a dried reaction tube was charged with 4,4,4′,4′,5,5,5′,5′-octamethyl-2,2′-bi (1,3,2-dioxaborolane) (233 mg, 918 μmol), bis((1Z,5Z)-cycloocta-1,5-diene) diiridiumbis(ylium)dimethanolate (20.9 mg, 31.5 μmol) and 4,4′-di-tert-butyl-2,2′bipyridyl (16.8 mg, 62.6 μmol). Anhydrous, degassed n-hexane (4 mL) was added and the reaction mixture was stirred at 20° C. for 0.5 h, then tert-butyl (2-(pyrimidin-2-yl)propan-2-yl)carbamate (150 mg, 96% wt, 607 μmol) was added. The sealed reaction mixture was then stirred at 70° C. for 16 h, poured into water (10 mL) and extracted with EtOAc (10 mL×3). The combined organics were washed with saturated aqueous NaCl (10 mL), dried over Na₂SO₄, filtered, and concentrated in vacuo. Flash chromatography (SiO₂, gradient 0-30% EtOAc/hexanes) provided the title compound (190 mg, 65%) as a pale yellow oil. 1H NMR (DMSO-d6) δ 8.86 (s, 1H), 7.93 (s, 2H), 1.53 (s, 6H), 1.32 (s, 9H), 1.16 (s, 12H).

### Preparation 449

### tert-Butyl (1-(5-(dimethylphosphoryl)-3-(trifluoromethyl)-1H-pyrazol-1-yl)-2-methylpropan-2-yl)carbamate

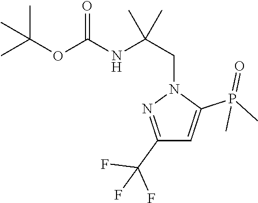 [SMILES: CC(C)(Cn1nc(C(F)(F)F)cc1P(C)(C)=O)NC(=O)OC(C)(C)C]

To a solution of dimethylphosphine oxide (0.34 g, 4.4 mmol) in 1,4-dioxane (10 mL) was added palladium diacetate (121 mg, 0.54 mmol), TEA (900 μL, 6.0 mmol), 1,1′-ferrocendiylbis(diphenylphosphine) (291 mg, 0.53 mmol) and tert-butyl (1-(5-bromo-3-(trifluoromethyl)-1H-pyrazol-1-yl)-2-methylpropan-2-yl)carbamate (1.0 g, 2.6 mmol) at 25° C. in a glovebox. The reaction mixture was removed from the glovebox and stirred at 110° C. for 4 h under N₂ under microwave irradiation. The reaction mixture was cooled to RT, filtered, and the filtrate was concentrated under reduced pressure. The residue was purified by column chromatography (SiO₂, 0-3% MeOH/DCM gradient) followed by reversed phase C18 flash chromatography (Spherical-C18 120 g, 5-60% ACN/0.5% aqueous formic acid) to afford the title compound (850 mg, 70%) as a yellow solid. ES/MS m/z 384.0 (M+H).

### Preparation 450

### tert-Butyl (2-methyl-1-(5-(1-methyl-1,2,3,6-tetrahydropyridin-4-yl)-3-(trifluoromethyl)-1H-pyrazol-1-yl)propan-2-yl)carbamate

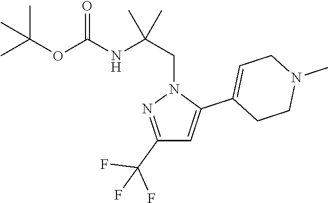 [SMILES: CN1CC=C(c2cc(C(F)(F)F)nn2CC(C)(C)NC(=O)OC(C)(C)C)CC1]

To a vial was added tert-butyl (1-(5-bromo-3-(trifluoromethyl)-1H-pyrazol-1-yl)-2-methylpropan-2-yl)carbamate (200 mg, 518 μmol), 1-methyl-4-(4,4,5,5-tetramethyl-1,3,2-dioxaborolan-2-yl)-1,2,3,6-tetrahydropyridine (231 mg, 1.04 mmol) and cesium carbonate (675 mg, 2.07 mmol), followed by 1,4-dioxane (2.0 mL) and H₂O (0.5 mL). The reaction mixture was de-gassed with N₂ for 3 min then, (1,1′-bis(diphenylphosphino)ferrocene)palladium(II) dichloride (42.3 mg, 51.8 μmol) was added and the reaction mixture was heated to 90° C. 5 h. The reaction mixture was cooled to RT and partitioned between EtOAc and H₂O. The layers were separated, and the aqueous layer was extracted with EtOAc (×2). The combined organic layer was dried over anhydrous Na₂SO₄, filtered and concentrated under reduced pressure. The residue was purified by flash column chromatography (SiO₂, 0-5% MeOH/DCM gradient) to afford the title compound (123 mg, 56%) as light brown solid. ES/MS m/z 403.2 (M+H).

### Preparation 451

### tert-Butyl (2-methyl-1-(5-(1-methylpiperidin-4-yl)-3-(trifluoromethyl)-1H-pyrazol-1-yl)propan-2-yl)carbamate

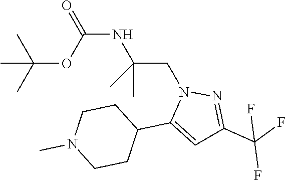 [SMILES: CN1CCC(c2cc(C(F)(F)F)nn2CC(C)(C)NC(=O)OC(C)(C)C)CC1]

To a solution of tert-butyl (2-methyl-1-(5-(1-methyl-1,2,3,6-tetrahydropyridin-4-yl)-3-(trifluoromethyl)-1H-pyrazol-1-yl)propan-2-yl)carbamate (123 mg 306 μmol) in MeOH (2 mL) was added 20% Palladium dihydroxide on Carbon (80 mg, 50% wt, 0.28 mmol) (50% wet). The reaction mixture was evacuated and filled N₂×2, followed by H₂×2. The reaction was allowed to stir at RT overnight. The reaction was filtered through a diatomaceous earth pad, washing with MeOH. The solvent was concentrated under reduced pressure to afford the title compound (91.4 mg, 74%) as an off-white solid. ES/MS m/z 405.2 (M+H).

### Preparation 452

### tert-Butyl (1-(5-carbamoyl-3-(trifluoromethyl)-1H-pyrazol-1-yl)-2-methylpropan-2-yl)carbamate

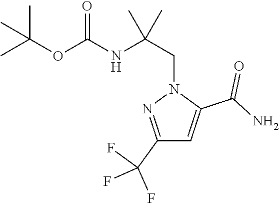 [SMILES: CC(C)(Cn1nc(C(F)(F)F)cc1C(N)=O)NC(=O)OC(C)(C)C]

To a solution of ethyl 1-(2-((tert-butoxycarbonyl)amino)-2-methylpropyl)-3-(trifluoromethyl)-1H-pyrazole-5-carboxylate (500 mg, 1.28 mmol) in EtOH (4 mL) was added ammonium hydroxide (4.00 mL, 25% wt, 25.8 mmol) and copper (II) sulfate anhydrous (20.5 mg, 128 μmol). The solution was stirred at 50° C. for 16 h. The reaction was diluted with H₂O (10 mL) and extracted with EtOAc (15 mL×3). The combined organic layer was washed with saturated aqueous NaCl (15 mL), dried over anhydrous Na₂SO₄, filtered and concentrated under reduced pressure. The residue was purified by flash column chromatography (SiO₂, 0-4% MeOH/DCM gradient) to afford the title compound (265 mg, 58%) as a white solid. ES/MS m/z 251.2 (M+H).

### Preparation 453

### tert-Butyl (1-(5-(aminomethyl)-3-(trifluoromethyl)-1H-pyrazol-1-yl)-2-methylpropan-2-yl)carbamate

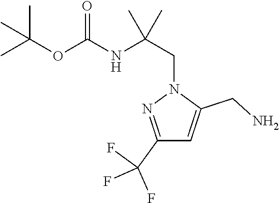 [SMILES: CC(C)(Cn1nc(C(F)(F)F)cc1CN)NC(=O)OC(C)(C)C]

A solution of tert-butyl (1-(5-carbamoyl-3-(trifluoromethyl)-1H-pyrazol-1-yl)-2-methylpropan-2-yl)carbamate (200 mg, 559 μmol) in THF (10 mL) was degassed and refilled with N₂×3. To the solution was added BH₃ in dimethyl sulfide (170 μL, 10 molar, 1.70 mmol) at 0° C. Then the reaction mixture was heated to 50° C. and stirred at 50° C. for 16 h. After 16 h, additional BH₃ in dimethyl sulfide (120 μL, 10 molar, 1.20 mmol) was added at RT and the reaction was stirred at 50° C. for an additional 16 h. The reaction was quenched by the additional MeOH (15 mL) and stirred at RT for 15 min. Then, the mixture was concentrated under reduced pressure. The residue was purified by flash column chromatography (SiO₂, 0-5% MeOH/DCM gradient) to afford the title compound (55 mg, 22%) as a colorless oil. ES/MS m/z 337.3 (M+H).

### Preparation 454

### tert-Butyl (2-methyl-1-(5-(methylsulfonamidomethyl)-3-(trifluoromethyl)-1H-pyrazol-1-yl)propan-2-yl)carbamate

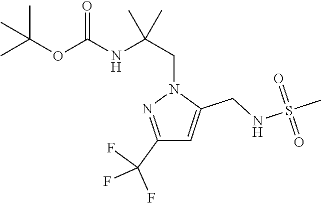 [SMILES: CC(C)(Cn1nc(C(F)(F)F)cc1CNS(C)(=O)=O)NC(=O)OC(C)(C)C]

To a solution of tert-butyl (1-(5-(aminomethyl)-3-(trifluoromethyl)-1H-pyrazol-1-yl)-2-methylpropan-2-yl)carbamate (55 mg, 0.12 mmol) and TEA (40 μL, 0.29 mmol) in DCM (2 mL) was added methanesulfonic anhydride (38 mg, 0.22 mmol) at 0° C. The reaction mixture was stirred at RT for 16 h. The reaction mixture was diluted with H₂O (15 mL) and extracted with DCM (15 mL×3). The combined organic layer was washed with saturated aqueous NaCl (10 mL), dried over anhydrous Na₂SO₄, filtered and concentrated under reduced pressure. The residue was purified by flash column chromatography (SiO₂, 0-4% MeOH/DCM gradient) to afford the title compound (51 mg, 61%) as a colorless oil. ES/MS m/z 415.3 (M+H).

### Preparation 455

### tert-Butyl (1-(3-chloro-1H-pyrazol-1-yl)-2-methylpropan-2-yl)carbamate

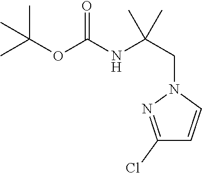 [SMILES: CC(C)(Cn1ccc(Cl)n1)NC(=O)OC(C)(C)C]

To a solution of 3-chloro-1H-pyrazole (2.0 g, 20 mmol) and tert-butyl 4,4-dimethyl-1,2,3-oxathiazolidine-3-carboxylate 2,2-dioxide (5.4 g, 21 mmol) in DMA (30 mL) was added cesium carbonate (13 g, 39 mmol). The mixture was stirred at 90° C. for 12 h. The reaction mixture was diluted with H₂O (100 mL) and extracted with EtOAc (100 mL×2). The combined organic layer was washed with saturated aqueous NaCl (100 mL×2), dried over Na₂SO₄, filtered and concentrated under reduced pressure. The residue was purified by flash column chromatography (SiO2, 0-1% MeOH/DCM gradient) to afford the title compound as an inseparable mixture with tert-butyl (1-(5-chloro-1H-pyrazol-1-yl)-2-methylpropan-2-yl)carbamate in a 10:1 ratio (4.4 g, 74%) as a white solid. ES/MS m/z 274.0/276.0 (M+H).

### Preparation 456

### tert-Butyl (1-(3,5-dibromo-1H-pyrazol-1-yl)-2-methylpropan-2-yl)carbamate

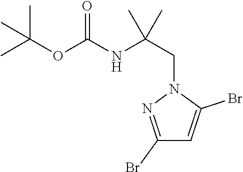 [SMILES: CC(C)(Cn1nc(Br)cc1Br)NC(=O)OC(C)(C)C]

The title compound was prepared as described in Preparation 455 using 3,5-dibromo-1H-pyrazole (10.2 g, 45.2 mmol), Cs₂CO₃ (43.7 g, 134 mmol), and tert-butyl 4,4-dimethyl-1,2,3-oxathiazolidine-3-carboxylate 2,2-dioxide (23.3 g, 92.7 mmol). The residue was purified by flash silica gel chromatography (0-100% DCM/hexanes gradient) to afford the title compound (19.2 g, 86%) as a white solid. ES/MS m/z 397.8 (M+H).

### Preparation 457

### tert-Butyl (1-(3-fluoro-1H-pyrazol-1-yl)-2-methylpropan-2-yl)carbamate

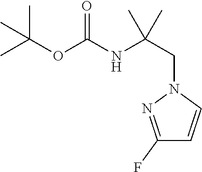 [SMILES: CC(C)(Cn1ccc(F)n1)NC(=O)OC(C)(C)C]

The title compound was prepared as described in Preparation 455 using tert-butyl 4,4-dimethyl-1,2,3-oxathiazolidine-3-carboxylate 2,2-dioxide (3.3 g, 13 mmol), 3-fluoro-1H-pyrazole (1.0 g, 12 mmol), and cesium carbonate (7.7 g, 24 mmol). The residue was purified by reverse phase HPLC (Phenomenex luna C18, 250×50 mm, 10 μm, gradient 30-70% ACN in 10 mM aq NH₄HCO₃) to provide the title compound (1.65 g, 52%) as a white solid. ES/MS m/z 258.1 (M+H).

### Preparation 458

### tert-Butyl (1-(3-chloro-5-(4,4,5,5-tetramethyl-1,3,2-dioxaborolan-2-yl)-1H-pyrazol-1-yl)-2-methylpropan-2-yl)carbamate

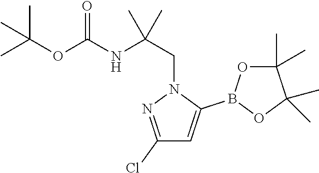 [SMILES: CC(C)(Cn1nc(Cl)cc1B1OC(C)(C)C(C)(C)O1)NC(=O)OC(C)(C)C]

In a N₂ filled glovebox, 4,4,4′,4′,5,5,5′,5′-octamethyl-2,2′-bi (1,3,2-dioxaborolane) (1.03 g, 4.06 mmol), bis((1Z,5Z)-cycloocta-1,5-diene)diiridiumbis(ylium)dimethanolate (66 mg, 0.10 mmol) and 4,4′-di-tert-butyl-2,2′-bipyridine (53 mg, 0.20 mmol) were added to a dried reaction vial. Dry, degassed n-hexane (10 mL) was added. Then the reaction mixture was stirred at 20° C. for 30 min. After 30 min, a solution of tert-butyl (1-(3-chloro-1H-pyrazol-1-yl)-2-methylpropan-2-yl)carbamate (1.00 g, 3.29 mmol) in n-hexane (5 mL) was added into the mixture. Then the sealed reaction vial was removed from the glovebox and stirred at 70° C. for 12 h. The reaction mixture was filtered. The filtrate was concentrated under reduced pressure to give a residue. The residue was purified by flash silica gel chromatography (0-10% EtOAc/hexanes gradient) to give the title compound (580 mg, 42%) as a pink solid. ES/MS m/z 400.0 (M+H).

### Preparation 459

### tert-Butyl (1-(3-fluoro-5-(4,4,5,5-tetramethyl-1,3,2-dioxaborolan-2-yl)-1H-pyrazol-1-yl)-2-methylpropan-2-yl)carbamate

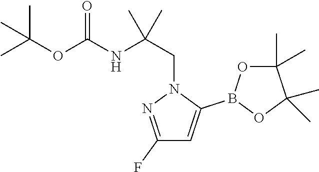 [SMILES: CC(C)(Cn1nc(F)cc1B1OC(C)(C)C(C)(C)O1)NC(=O)OC(C)(C)C]

The title compound was prepared as described in Preparation 458 using 4,4,4′,4′,5,5,5′,5′-octamethyl-2,2′-bi (1,3,2-dioxaborolane) (1.1 g, 4.3 mmol), bis((1Z,5Z)-cycloocta-1,5-diene)diiridiumbis(ylium)dimethanolate (75 mg, 0.11 mmol), and 4,4′-di-tert-butyl-2,2′-bipyridine (60 mg, 0.22 mmol). The residue was purified by flash silica gel chromatography (100% DCM) to give the title compound (645 mg, 41%) as a yellow solid. ES/MS m/z 384.0 (M+H).

### Preparation 460

### tert-Butyl (1-(3-chloro-5-(2-methylpyrimidin-5-yl)-1H-pyrazol-1-yl)-2-methylpropan-2-yl)carbamate

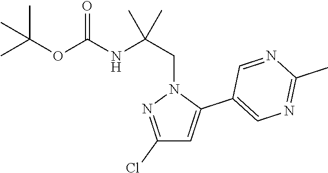 [SMILES: Cc1ncc(-c2cc(Cl)nn2CC(C)(C)NC(=O)OC(C)(C)C)cn1]

To a solution of tert-butyl (1-(3-chloro-5-(4,4,5,5-tetramethyl-1,3,2-dioxaborolan-2-yl)-1H-pyrazol-1-yl)-2-methylpropan-2-yl)carbamate (200 mg, 475 μmol), 5-bromo-2-methylpyrimidine (100 mg, 578 μmol) and cesium carbonate (310 mg, 951 μmol) in 1,4-dioxane (5 mL) and water (0.5 mL) was added 1,1′-bis(di-t-butylphosphino)ferrocene palladium dichloride (62 mg, 95 μmol). The reaction mixture was degassed and purged with N₂ 3 times, then stirred at 90° C. for 12 h under N₂. The reaction mixture was filtered. The filtrate was concentrated under reduced pressure to give a residue. The residue was purified by flash silica gel chromatography (0-53% EtOAc/hexanes gradient) to provide the title compound (130 mg, 71%) as a yellow solid. ES/MS m/z 366.0 (M+H).

### Preparation 461

### tert-Butyl (1-(3-fluoro-5-(2-methylpyrimidin-5-yl)-1H-pyrazol-1-yl)-2-methylpropan-2-yl)carbamate

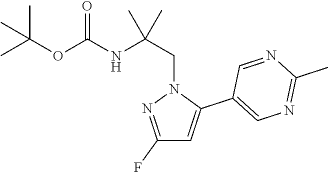 [SMILES: Cc1ncc(-c2cc(F)nn2CC(C)(C)NC(=O)OC(C)(C)C)cn1]

The title compound was prepared as described in Preparation 460 using tert-butyl (1-(3-fluoro-5-(4,4,5,5-tetramethyl-1,3,2-dioxaborolan-2-yl)-1H-pyrazol-1-yl)-2-methylpropan-2-yl)carbamate (200 mg, 470 μmol), 5-bromo-2-methylpyrimidine (98 mg, 0.57 mmol), cesium carbonate (310 mg, 951 μmol), and 1,1′-bis(di-t-butylphosphino)ferrocene palladium dichloride (62 mg, 95 μmol). The residue was purified by flash silica gel chromatography (0-50% EtOAc/hexanes) to give the title compound (180 mg, 93%) as a yellow solid. ES/MS m/z 350.1 (M+H).

### Preparation 462

### tert-Butyl (1-(3-bromo-5-(2-methylpyrimidin-5-yl)-1H-pyrazol-1-yl)-2-methylpropan-2-yl)carbamate

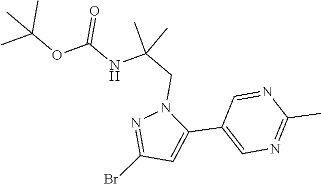 [SMILES: Cc1ncc(-c2cc(Br)nn2CC(C)(C)NC(=O)OC(C)(C)C)cn1]

A mixture of tert-butyl (1-(3,5-dibromo-1H-pyrazol-1-yl)-2-methylpropan-2-yl)carbamate (8.01 g, 16.1 mmol), (2-methylpyrimidin-5-yl)boronic acid (1.58 g, 11.5 mmol), Na₂CO₃ (5.20 g, 49.1 mmol) and PdCl₂(dppf) (1.22 g, 1.67 mmol) in 1,4-dioxane (30 mL) and water (3 mL) was degassed and purged with N₂ 3 times. Then the reaction mixture was stirred at 90° C. for 16 h under N₂. The mixture was diluted with water (60 mL) and extracted with EtOAc (80 mL×2). The combined organic layers were dried over anhydrous Na₂SO₄, filtered and concentrated in vacuo to give a residue. The residue was purified by flash silica gel chromatography (0-40% EtOAc/hexanes) to give a residue, then further purified by prep-HPLC (column: Phenomenex luna C18 250*50 mm*10 mm; mobile phase: A=H₂O (10 mM NH₄HCO₃); B=ACN; B % 30-70%, 20 min; flow rate: 100 mL/min) to afford the title compound (1.2 g, 18%) as a white solid. ES/MS m/z 410.1 (M+H).

### Preparation 463

### tert-Butyl (1-(3-cyclopropyl-5-(2-methylpyrimidin-5-yl)-1H-pyrazol-1-yl)-2-methylpropan-2-yl)carbamate

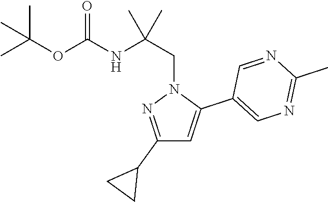 [SMILES: Cc1ncc(-c2cc(C3CC3)nn2CC(C)(C)NC(=O)OC(C)(C)C)cn1]

A RBF containing tert-butyl (1-(3-bromo-5-(2-methylpyrimidin-5-yl)-1H-pyrazol-1-yl)-2-methylpropan-2-yl)carbamate (122 mg, 291 μmol, 98% purity), cyclopropylboronic acid (80.5 mg, 937 μmol), K₃PO₄ (194 mg, 914 μmol). Pd(OAc)₂ (12.7 mg, 56.6 μmol), tricyclohexylphosphine (42.4 mg, 151 μmol), toluene (5 mL), and H₂O (0.5 mL) was evacuated and backfilled with N₂×3. The mixture was stirred at 100° C. for 12 h, diluted with H₂O (15 mL) and extracted with EtOAc (15 mL×2). The combined organic layers were washed with saturated aqueous NaCl (10 mL), dried over Na₂SO₄, filtered, and concentrated in vacuo. Flash chromatography (SiO₂, gradient 0-25% EtOAc in hexanes) provided the title compound (98.5 mg, 87%) as a colorless oil. ES-MS m/z 372.4 (M+H).

### Preparation 464

### tert-Butyl (2-methyl-1-(3-methyl-5-(2-methylpyrimidin-5-yl)-1H-pyrazol-1-yl)propan-2-yl)carbamate and tert-butyl (2-methyl-1-(5-(2-methylpyrimidin-5-yl)-1H-pyrazol-1-yl)propan-2-yl)carbamate (Mixture)

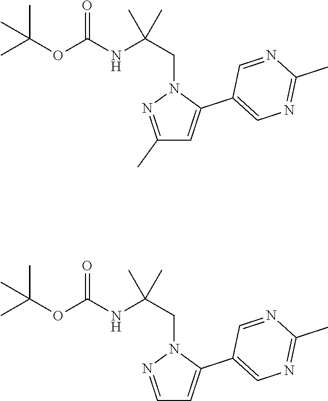

To a solution of tert-butyl (1-(3-bromo-5-(2-methylpyrimidin-5-yl)-1H-pyrazol-1-yl)-2-methylpropan-2-yl)carbamate (300 mg, 717 μmol, 98% purity) in 1,4-dioxane (5 mL) were added methylboronic acid (133 mg, 2.22 mmol), Na₂CO₃ (232 mg, 2.19 mmol), PdCl₂(dppf) (54 mg, 74 μmol) and H₂O (0.5 mL). The reaction vessel was evacuated and backfilled with N₂, then stirred at 90° C. for 16 h, diluted with H₂O (10 mL) and extracted with EtOAc (10 mL×2). The combined organic layers were dried over Na₂SO₄, filtered, and concentrated in vacuo. Flash chromatography (SiO₂, gradient 0-40% EtOAc in hexanes) provided the title compounds as an inseparable 7:1 mixture favoring tert-butyl (2-methyl-1-(3-methyl-5-(2-methylpyrimidin-5-yl)-1H-pyrazol-1-yl)propan-2-yl)carbamate (194 mg combined, approximately 76% combined yield) as a white solid. For tert-butyl (2-methyl-1-(3-methyl-5-(2-methylpyrimidin-5-yl)-1H-pyrazol-1-yl)propan-2-yl)carbamate: ES-MS m/z 346.1 (M+H). For tert-butyl (2-methyl-1-(5-(2-methylpyrimidin-5-yl)-1H-pyrazol-1-yl)propan-2-yl)carbamate: ES-MS m/z 332.1 (M+H).

### Preparation 465

### 2-Methyl-2-nitro-1-(4-(trifluoromethyl)-1H-imidazol-2-yl)propan-1-ol (racemic mixture)

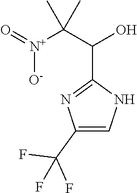 [SMILES: CC(C)(C(O)c1nc(C(F)(F)F)c[nH]1)[N+](=O)[O-]]

To a solution of 2-nitropropane (7.0 g, 79 mmol) and lithium methoxide (1 M in MeOH) (40 mL, 40 mmol) in MeOH (30 mL) was added 4-(trifluoromethyl)-1H-imidazole-2-carbaldehyde (3.0 g, 18 mmol). The reaction mixture was stirred at 20° C. for 12 h. The mixture was diluted with water (30 mL) and extracted with EtOAc (60 mL×2). The organic layers were combined, dried over Na₂SO₄, filtered and concentrated under reduced pressure. The residue was purified by flash silica gel chromatography (0-8% MeOH in DCM gradient) followed by trituration with EtOAc (30 mL) at 20° C. for 10 min. The mixture was filtered, and the filtrate was concentrated under reduced pressure. The residue was triturated with DCM (30 mL) at 20° C. for 10 min. The mixture was filtered to give a cake, which was dried under reduced pressure to afford the title compound (1.3 g, 28%) as a white solid. ES/MS m/z 254.0 (M+H).

### Preparation 466

### 2-amino-2-methyl-1-(4-(trifluoromethyl)-1H-imidazol-2-yl)propan-1-ol (racemic mixture)

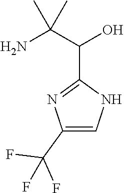

A mixture of 2-methyl-2-nitro-1-(4-(trifluoromethyl)-1H-imidazol-2-yl)propan-1-ol (racemic mixture) (1.3 g, 5.1 mmol) in MeOH (15 mL) was treated with zinc (3.63 g, 55.5 mmol) and HCl (4 M in water) (15 mL, 60 mmol). After stirring at 20° C. for 12 h, the reaction mixture was diluted with MeOH (20 mL) and filtered. The filter cake was washed with MeOH (30 mL×3). The filtrate was concentrated under reduced pressure to give the title compound (2.5 g, 99+% yield) as a white solid. ES/MS m/z 224.1 (M+H).

### Preparation 467

### tert-Butyl (1-hydroxy-2-methyl-1-(4-(trifluoromethyl)-1H-imidazol-2-yl)propan-2-yl)carbamate (Racemic Mixture)

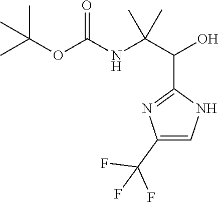 [SMILES: CC(C)(C)OC(=O)NC(C)(C)C(O)c1nc(C(F)(F)F)c[nH]1]

To a mixture of 2-amino-2-methyl-1-(4-(trifluoromethyl)-1H-imidazol-2-yl)propan-1-ol (racemic mixture) (2.5 g, 46% purity, 5.2 mmol) and TEA (2.3 mL, 17 mmol) in THF (20 mL) was added di-tert-butyl dicarbonate (2.3 mL, 2.2 g, 10 mmol). After stirring at 20° C. for 12 h, the reaction mixture was treated with additional di-tert-butyl dicarbonate (2.5 mL, 2.4 g, 11 mmol) and stirring was continued for additional 12 h. The mixture was diluted with water (100 mL) and extracted with EtOAc (100 mL×2). The organic layers were combined, dried over Na₂SO₄, filtered and concentrated under reduced pressure. The residue was purified by flash silica gel chromatography (0-3% MeOH in DCM gradient) to obtain the title compound (1.3 g, 50%) as a white solid. ES/MS m/z 324.0 (M+H).

### Preparation 468

### 2-Methyl-1-(5-(1-methylpyrrolidin-3-yl)-3-(trifluoromethyl)-1H-pyrazol-1-yl)propan-2-amine dihydrochloride (racemic mixture)

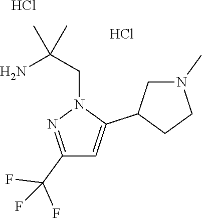 [SMILES: Cl ; CN1CCC(c2cc(C(F)(F)F)nn2CC(C)(C)N)C1 ; Cl]

A solution of 3-(1-(2-amino-2-methylpropyl)-3-(trifluoromethyl)-1H-pyrazol-5-yl)-1-methylpyrrolidin-2-one (racemic mixture) (271 mg, 95% purity, 846 μmol) in THF (10 mL) was degassed and purged with N₂ (3×) then cooled to 0° C. LiAlH₄ (2.5 M in THF) (1.2 mL, 3.0 mmol) was added, and the resulting mixture was stirred at 0° C. for 1 h under N₂. Water (120 μL), 15% aq NaOH (120 μL), and water (360 μL) were added sequentially then the cooling bath was removed. The reaction mixture was stirred at 20° C. for 0.5 h, then concentrated under reduced pressure. The residue was diluted with MeOH (10 mL) and filtered. The filtrate was concentrated under reduced pressure. The crude residue was combined with the crude residue obtained from a second reaction run on 50 mg scale of 3-(1-(2-amino-2-methylpropyl)-3-(trifluoromethyl)-1H-pyrazol-5-yl)-1-methylpyrrolidin-2-one. Purification by reversed phase HPLC (Welch Xtimate® C18, 40×200 mm, 7 μm, gradient 0-30% ACN in water, with 0.1% TFA) provided 2-methyl-1-(5-(1-methylpyrrolidin-3-yl)-3-(trifluoromethyl)-1H-pyrazol-1-yl)propan-2-amine bis-trifluoroacetate (racemic mixture) (197 mg, 32%) as a colorless oil. ES-MS m/z 291.1 (M+H). 2-methyl-1-(5-(1-methylpyrrolidin-3-yl)-3-(trifluoromethyl)-1H-pyrazol-1-yl)propan-2-amine bis-trifluoroacetate (racemic mixture) (60 mg, 85% purity, 10 μmol) was diluted with HCl (1M in water) (0.5 mL) and ACN (1 mL). The mixture was frozen and lyophilized. Repeated dilution and lyophilization (2 times) gave a quantitative yield of the title compound (48 mg) as a yellow solid. ES-MS m/z 291.1 (M+H).

### Preparation 469

### Benzyl (1-(5-bromo-3-(trifluoromethyl)-1H-pyrazol-1-yl)-2-methylpropan-2-yl)carbamate

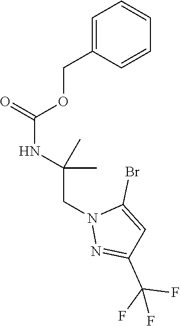 [SMILES: CC(C)(Cn1nc(C(F)(F)F)cc1Br)NC(=O)OCc1ccccc1]

A suspension of 1-(5-bromo-3-(trifluoromethyl)-1H-pyrazol-1-yl)-2-methylpropan-2-amine hydrochloride (500 mg, 1.55 mmol) and sodium bicarbonate (326 mg, 3.88 mmol) in DCM (4.43 mL) and water (738 μL) was treated with benzyl chloroformate (709 μL, 4.65 mmol), and the resulting mixture was stirred at RT overnight. The reaction mixture was diluted with DCM (10 mL) and water (10 mL). The layers were separated, and the aqueous layer was extracted with DCM (2×5 mL). The organic layers were combined and dried under a stream of nitrogen. The residue was purified by flash silica gel chromatography (0-30% MTBE in heptane gradient) to obtain the title compound (622.6 mg, 96%) as a white solid. ES/MS m/z (⁷⁹Br/⁸¹Br) 420.2/422.2 (M+H).

### Preparation 470

### tert-Butyl 2-(1-(2-(((benzyloxy)carbonyl)amino)-2-methylpropyl)-3-(trifluoromethyl)-1H-pyrazol-5-yl)-1H-pyrrole-1-carboxylate

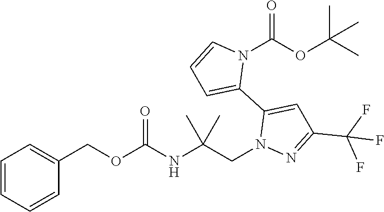 [SMILES: CC(C)(Cn1nc(C(F)(F)F)cc1-c1cccn1C(=O)OC(C)(C)C)NC(=O)OCc1ccccc1]

A mixture of benzyl (1-(5-bromo-3-(trifluoromethyl)-1H-pyrazol-1-yl)-2-methylpropan-2-yl)carbamate (158.6 mg, 377.4 μmol), Pd (dppf) Cl₂·CH₂Cl₂ (30.8 mg, 37.7 μmol), and (1-(tert-butoxycarbonyl)-1H-pyrrol-2-yl)boronic acid (120 mg, 569 μmol) was suspended in 1,4-dioxane (2.5 mL) and then a solution of Cs₂CO₃ (369 mg, 1.13 mmol) in water (680 μL) was added. The resulting mixture was sparged with nitrogen for 10 min and then stirred at 80° C. under an atmosphere of nitrogen for 24 h. The reaction was then cooled to RT and diluted with EtOAc (5 mL) and sat. aq. NaHCO₃ (3 mL). The layers were separated and the aqueous layer extracted with EtOAc (2×3 mL). The combined organic layers were dried under a stream of nitrogen and purified by silica gel chromatography using a gradient of 0-50% MTBE in heptane to afford the title compound (121.2 mg, 63%) as a colorless oil. ES-MS m/z 507.4 (M+H).

### Preparation 471

### Benzyl (1-(5-(((tert-butoxycarbonyl)amino)methyl)-3-(trifluoromethyl)-1H-pyrazol-1-yl)-2-methylpropan-2-yl)carbamate

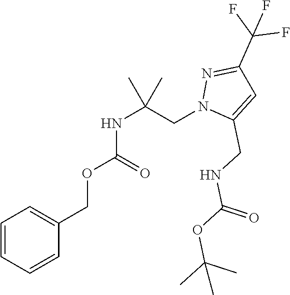 [SMILES: CC(C)(Cn1nc(C(F)(F)F)cc1CNC(=O)OC(C)(C)C)NC(=O)OCc1ccccc1]

A mixture of benzyl (1-(5-bromo-3-(trifluoromethyl)-1H-pyrazol-1-yl)-2-methylpropan-2-yl)carbamate (100 mg, 238 μmol), Pd(dppf)Cl₂·CH₂Cl₂ (19 mg, 23 μmol), and potassium (((tert-butoxycarbonyl)amino)methyl)trifluoroborate (282 mg, 1.19 mmol) were suspended in 1,4-dioxane (1.59 mL) and a solution of Cs₂CO₃ (233 mg, 715 μmol) in water (429 μL) was added. The resulting mixture was sparged with nitrogen for 10 min and then stirred at 80° C. under an atmosphere of nitrogen overnight, cooled to RT and diluted with EtOAc (10 mL) and sat. aq. NaHCO₃ (5 mL). The layers were separated and the aqueous layer extracted with EtOAc (2×3 mL). The combined organic layers were passed through a plug of Na₂SO₄, dried under a stream of nitrogen, and purified by silica gel chromatography using a gradient of 0-50% MTBE in heptane to afford the title compound (14.5 mg, 13%) as a colorless film. ES-MS m/z 415.2 m/z [M+H-tBu].

### Preparation 472

### rac-tert-Butyl (R)-2-(1-(2-amino-2-methylpropyl)-3-(trifluoromethyl)-1H-pyrazol-5-yl)pyrrolidine-1-carboxylate

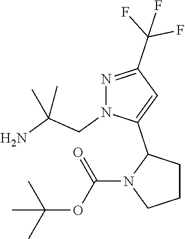 [SMILES: CC(C)(N)Cn1nc(C(F)(F)F)cc1C1CCCN1C(=O)OC(C)(C)C]

A solution of tert-butyl 2-(1-(2-(((benzyloxy)carbonyl)amino)-2-methylpropyl)-3-(trifluoromethyl)-1H-pyrazol-5-yl)-1H-pyrrole-1-carboxylate (121.2 mg, 239.3 μmol) in EtOH (4.8 mL) was sparged with nitrogen for 5 min. Then Pd on carbon (51 mg, 20% w/w loading, 50% wetted, 10% wt overall, 48 μmol) was added, the resulting mixture was sparged with nitrogen for 5 min, and then the nitrogen line was replaced with a hydrogen balloon. The mixture was sparged with hydrogen for 5 min, then the vent needle was removed and the mixture stirred at RT under balloon pressure of hydrogen. After 2 h, the vial was purged with nitrogen and then the reaction mixture was filtered through a plug of diatomaceous earth, rinsing with additional EtOH. The filtrate was concentrated under a stream of nitrogen to afford the title compound (83.0 mg, 92%) as a white crystalline solid. ES-MS m/z 377.2 [M+H].

### Preparation 473

### tert-Butyl ((1-(2-amino-2-methylpropyl)-3-(trifluoromethyl)-1H-pyrazol-5-yl)methyl)carbamate

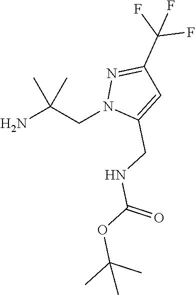 [SMILES: CC(C)(N)Cn1nc(C(F)(F)F)cc1CNC(=O)OC(C)(C)C]

A solution of benzyl (1-(5-(((tert-butoxycarbonyl)amino)methyl)-3-(trifluoromethyl)-1H-pyrazol-1-yl)-2-methylpropan-2-yl)carbamate (14.5 mg, 30.8 μmol) in EtOH (616 μL) was sparged with nitrogen for 5 min. Then Pd on carbon (6.6 mg, 20% w/w loading, 50% wetted, 10% wt overall, 6.2 μmol) was added, the resulting mixture was sparged with nitrogen for 5 min, and then the nitrogen line was replaced with a hydrogen balloon. The mixture was sparged with hydrogen for 5 min, then the vent needle was removed and the mixture stirred at RT under balloon pressure of hydrogen. After 2 h, the vial was purged with nitrogen and then the reaction mixture was filtered through a plug of diatomaceous earth pre-wetted with EtOH, rinsing with additional EtOH. The filtrate was concentrated under a stream of nitrogen to afford the title compound (10.4 mg, 99+%) as a white solid. ES-MS m/z 337.2 [M+H].

### Preparation 474

### (4-(1-(2-Amino-2-methylpropyl)-3-(trifluoromethyl)-1H-pyrazol-5-yl)phenyl)dimethylphosphine oxide hydrochloride

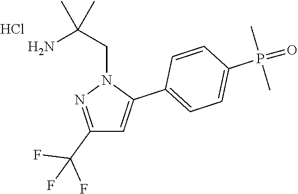 [SMILES: Cl ; CC(C)(N)Cn1nc(C(F)(F)F)cc1-c1ccc(P(C)(C)=O)cc1]

To a solution of tert-butyl (1-(5-(4-(dimethylphosphoryl)phenyl)-3-(trifluoromethyl)-1H-pyrazol-1-yl)-2-methylpropan-2-yl)carbamate (143.3 mg, 95% wt, 296.3 μmol) in 1,4-dioxane (1 mL) was added a solution of HCl in dioxane (1.5 mL of a 2 M solution, 3.0 mmol). The resulting mixture was stirred at 28° C. After 3 h, the reaction was concentrated under reduced pressure to afford the title compound (153.2 mg, 99+%) as a white solid. ES-MS m/z 360.3 [M-Cl].

The compounds in the following table were prepared in similar manner as described in Preparation 474 using the appropriate Boc protected amine. Reactants can be added in different orders or in differing equivalency, solvents can be differing ratios and mixtures of ethers and alcohols, reactions can be run for 1-72 hours, and purification methods were adjusted to suit the compounds. Such variances would be apparent to one skilled in the art.

TABLE 43

| Preparation | | | ES-MS |
|---|---|---|---|
| No. | Chemical Name | Structure | m/z |
| 475 | (4-(1-(2-Amino-2- methylpropyl)-3- (trifluoromethyl)-1H- pyrazol-5-yl)-2- (methylamino)phenyl) diethylphosphine oxide dihydrochloride | 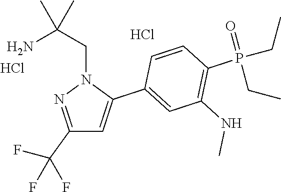 [SMILES: Cl ; CCP(=O)(CC)c1ccc(-c2cc(C(F)(F)F)nn2CC(C)(C)N)cc1NC ; Cl] | 417.2 (M + H) |
| 476 | (3-(1-(2-Amino-2- methylpropyl)-3- (trifluoromethyl)-1H- pyrazol-5- yl)phenyl)dimethyl- phosphine oxide hydrochloride | 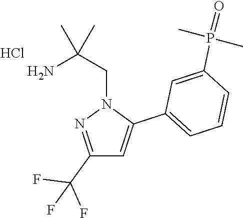 [SMILES: Cl ; CC(C)(N)Cn1nc(C(F)(F)F)cc1-c1cccc(P(C)(C)=O)c1] | 360.0 (M + H) |
| 477 | (1-(2-Amino-2- methylpropyl)-3- (trifluoromethyl)-1H- pyrazol-5- yl)dimethylphosphine oxide hydrochloride | 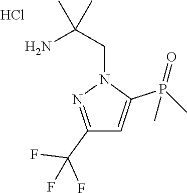 [SMILES: Cl ; CC(C)(N)Cn1nc(C(F)(F)F)cc1P(C)(C)=O] | 284.3 (M + H) |
| 478 | 2-Methyl-1-(5-(1- methylpiperidin-4-yl)-3- (trifluoromethyl)-1H- pyrazol-1-yl)propan-2- amine dihydrochloride | 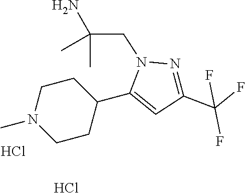 [SMILES: CN1CCC(c2cc(C(F)(F)F)nn2CC(C)(C)N)CC1 ; Cl ; Cl] | 305.0 (M + H) |
| 479 | N-((1-(2-Amino-2- methylpropyl)-3- (trifluoromethyl)-1H- pyrazol-5- yl)methyl)methane- sulfonamide hydrochloride | 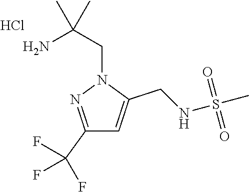 [SMILES: Cl ; CC(C)(N)Cn1nc(C(F)(F)F)cc1CNS(C)(=O)=O] | 315.2 (M + H) |
| 480 | 1-(3-chloro-5-(2- methylpyrimidin-5-yl)- 1H-pyrazol-1-yl)-2- methylpropan-2-amine dihydrochloride | 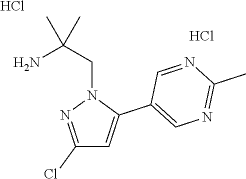 [SMILES: Cl ; Cc1ncc(-c2cc(Cl)nn2CC(C)(C)N)cn1 ; Cl] | 266.0, 267.9 (M + H) |
| 481 | 1-(3-Fluoro-5-(2- methylpyrimidin-5-yl)- 1H-pyrazol-1-yl)-2- methylpropan-2-amine dihydrochloride | 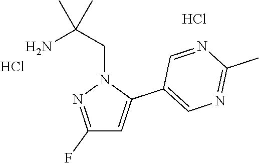 [SMILES: Cl ; Cc1ncc(-c2cc(F)nn2CC(C)(C)N)cn1 ; Cl] | 250.1 (M + H) |
| 482 | 1-(3-Cyclopropyl-5-(2- methylpyrimidin-5-yl)- 1H-pyrazol-1-yl)-2- methylpropan-2-amine dihydrochloride | 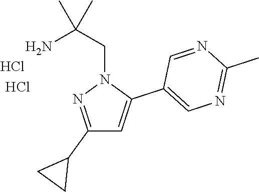 [SMILES: Cl ; Cl ; Cc1ncc(-c2cc(C3CC3)nn2CC(C)(C)N)cn1] | 272.1 (M + H) |
| 483 | 2-Amino-2-methyl-1-(4- (trifluoromethyl)-1H- imidazol-2-yl)propan-1- ol hydrochloride | 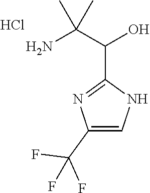 | 224.1 (M + H) |

### Preparation 484

### 2-Methyl-1-(3-methyl-5-(2-methylpyrimidin-5-yl)-1H-pyrazol-1-yl)propan-2-amine dihydrochloride and 2-Methyl-1-(5-(2-methylpyrimidin-5-yl)-1H-pyrazol-1-yl)propan-2-amine dihydrochloride

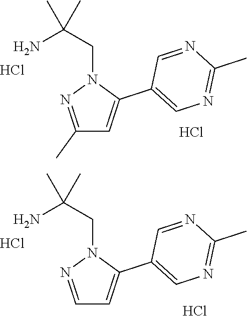

The title compounds were prepared according to Preparation 474 using a mixture of tert-butyl (2-methyl-1-(3-methyl-5-(2-methylpyrimidin-5-yl)-1H-pyrazol-1-yl)propan-2-yl)carbamate and tert-butyl (2-methyl-1-(5-(2-methylpyrimidin-5-yl)-1H-pyrazol-1-yl)propan-2-yl)carbamate (˜7:1 ratio, 194 mg, 94% wt, 269 μmol) as the starting material. This was combined with a second batch prepared according to Preparation 474 using a mixture of tert-butyl (2-methyl-1-(3-methyl-5-(2-methylpyrimidin-5-yl)-1H-pyrazol-1-yl)propan-2-yl)carbamate and tert-butyl (2-methyl-1-(5-(2-methylpyrimidin-5-yl)-1H-pyrazol-1-yl)propan-2-yl)carbamate (˜3.5:1 ratio, 51 mg, 95% wt, 72 μmol) as the starting material, and then the combined crude products were purified by reverse-phase HPLC (F-Welch Xtimate C18 40*200 mm 7 μm; using a gradient of 0%-10% ACN in 0.05% HCl/water with a flow rate of 60 mL/min). 2-Methyl-1-(3-methyl-5-(2-methylpyrimidin-5-yl)-1H-pyrazol-1-yl)propan-2-amine dihydrochloride (122 mg, 56%) was obtained as a white solid. ES-MS m/z 246.2 (M+H−2HCl). 2-Methyl-1-(5-(2-methylpyrimidin-5-yl)-1H-pyrazol-1-yl)propan-2-amine dihydrochloride (180 mg, 18%) was obtained as a white solid. ES-MS m/z 232.2 (M+H−2HCl).

### Example 168

### (R)—N-(1-(5-Bromo-3-(trifluoromethyl)-1H-pyrazol-1-yl)-2-methylpropan-2-yl)-2-(5-(3-chloro-6-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propenamide

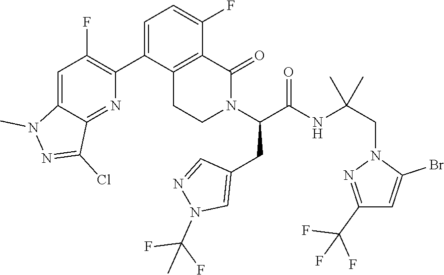 [SMILES: Cn1nc(Cl)c2nc(-c3ccc(F)c4c3CCN([C@H](Cc3cnn(C(C)(F)F)c3)C(=O)NC(C)(C)Cn3nc(C(F)(F)F)cc3Br)C4=O)c(F)cc21]

To a mixture of (R)-2-(5-(3-chloro-6-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (350 mg, 91 wt %, 593 μmol), 1-(5-bromo-3-(trifluoromethyl)-1H-pyrazol-1-yl)-2-methylpropan-2-amine hydrochloride (195 mg, 605 μmol), and DEPBT (360 mg, 1.20 mmol) in DMA (5 mL) was added 4-methylmorpholine (0.65 mL, 5.9 mmol). The mixture was stirred at RT for 6 h. The reaction mixture was diluted with water (50 mL) and extracted with EtOAc (50 mL×3). The combined organic layers were washed with saturated aqueous NaCl (40 mL×2), dried over Na₂SO₄, filtered, and the filtrate concentrated under reduced pressure. The residue was purified by column chromatography (SiO₂, 0-60% EtOAc/hexanes gradient) to afford the title compound (390 mg, 74%) as a white solid. ES/MS m/z 803.7/805.5/807.8 (M+H), Cl and Br isotope.

The compounds in the following table were prepared in similar manner as described in Example 168 using the appropriate carboxylic acid and primary amine described above. Reactants can be added in different orders or in differing equivalency, reactions can be run for 1-24 hours, temperature ranged from 0° C. to room temperature, and purification methods were adjusted to suit the compounds. Such variances would be apparent to one skilled in the art.

TABLE 44

| Preparation/ | | | ES-MS |
|---|---|---|---|
| Example | Chemical Name | Structure | m/z |
| Ex. 169 | (R)-N-(1-(5-Bromo-3- (trifluoromethyl)-1H- pyrazol-1-yl)-2- methylpropan-2-yl)-2- (5-(3,6-difluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide | 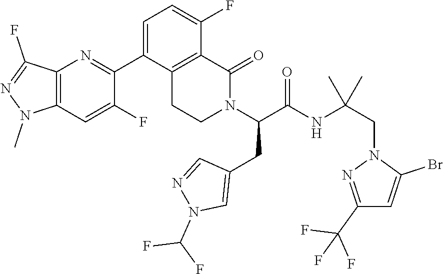 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3Br)C4=O)c(F)cc21] | 788.2/ 790.2 (M + H) |
| Prep. 487 | tert-Butyl-2-(1-(2-((R)- 3-(1-(difluoromethyl)- 1H-pyrazol-4-yl)-2-(8- fluoro-5-(6-fluoro-1- methyl-1H- pyrazolo[4,3-b]pyridin- 5-yl)-1-oxo-3,4- dihydroisoquinolin- 2(1H)-yl)propanamido)- 2-methylpropyl)-3- (trifluoromethyl)-1H- pyrazol-5-yl)pyrrolidine- 1-carboxylate (Diastereomer Mixture) | 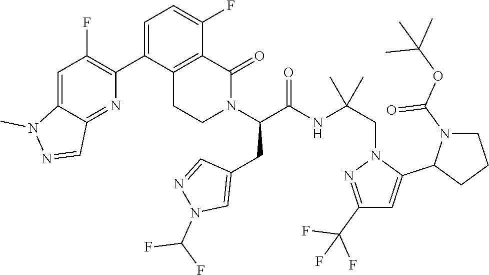 [SMILES: Cn1ncc2nc(-c3ccc(F)c4c3CCN([C@H](Cc3cnn(C(F)F)c3)C(=O)NC(C)(C)Cn3nc(C(F)(F)F)cc3C3CCCN3C(=O)OC(C)(C)C)C4=O)c(F)cc21] | 861.4 (M + H) |
| Prep. 488 | Benzyl-2-(1-(2-((R)-3- (1-(difluoromethyl)-1H- pyrazol-4-yl)-2-(8- fluoro-5-(6-fluoro-1- methyl-1H- pyrazolo[4,3-b]pyridin- 5-yl)-1-oxo-3,4- dihydroisoquinolin- 2(1H)-yl)propanamido)- 2-methylpropyl)-3- (trifluoromethyl)-1H- pyrazol-5-yl)azetidine-1- carboxylate (Diastereomer 1) | 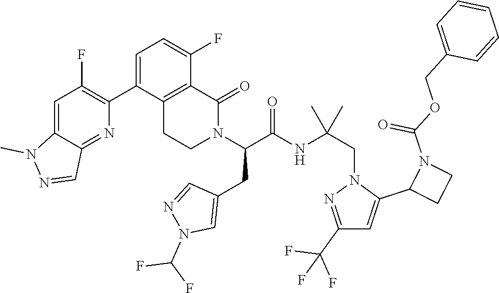 [SMILES: Cn1ncc2nc(-c3ccc(F)c4c3CCN([C@H](Cc3cnn(C(F)F)c3)C(=O)NC(C)(C)Cn3nc(C(F)(F)F)cc3C3CCN3C(=O)OCc3ccccc3)C4=O)c(F)cc21] | 881.4 (M + H) |
| Prep. 489 | tert-Butyl (R)-((1-(2-(3- (1-(difluoromethyl)-1H- pyrazol-4-yl)-2-(8- fluoro-5-(6-fluoro-1- methyl-1H- pyrazolo[4,3-b]pyridin- 5-yl)-1-oxo-3,4- dihydroisoquinolin- 2(1H)-yl)propanamido)- 2-methylpropyl)-3- (trifluoromethyl)-1H- pyrazol-5- yl)methyl)carbamate | 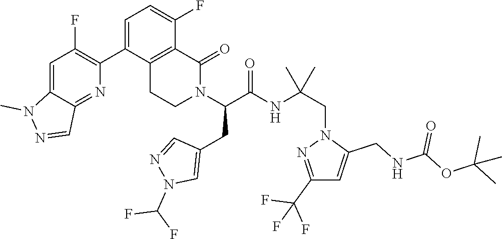 [SMILES: Cn1ncc2nc(-c3ccc(F)c4c3CCN([C@H](Cc3cnn(C(F)F)c3)C(=O)NC(C)(C)Cn3nc(C(F)(F)F)cc3CNC(=O)OC(C)(C)C)C4=O)c(F)cc21] | 821.4 (M + H) |

### Preparation 490

### tert-Butyl (R)-(2-(5-(1-(2-(2-(5-(3-chloro-6-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)carbamate

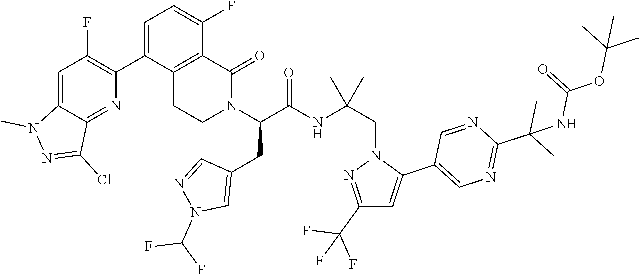 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)NC(=O)OC(C)(C)C)nc3)C4=O)c(F)cc21]

To a solution of (R)—N-(1-(5-bromo-3-(trifluoromethyl)-1H-pyrazol-1-yl)-2-methylpropan-2-yl)-2-(5-(3-chloro-6-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propenamide (100 mg, 90% wt, 112 μmol), tert-butyl (2-(5-(4,4,5,5-tetramethyl-1,3,2-dioxaborolan-2-yl)pyrimidin-2-yl)propan-2-yl)carbamate (62 mg, 75% wt, 0.13 mmol), and Na₂CO₃ (38 mg, 0.36 mmol) in 1,4-dioxane (3 mL) and water (0.3 mL) was added XPhos Pd G3 (19.3 mg, 22.8 μmol). The mixture was degassed and backfilled with N₂ (×3), then the mixture was stirred at 90° C. under nitrogen. Additional charges of tert-butyl (2-(5-(4,4,5,5-tetramethyl-1,3,2-dioxaborolan-2-yl)pyrimidin-2-yl)propan-2-yl)carbamate (33 mg, 75% wt, 68 μmol), Na₂CO₃ (26 mg, 0.25 mmol), and XPhos Pd G3 (9.8 mg, 12 μmol) were made after 1 h and 24 h of heating. The reaction mixture was degassed and backfilled with N₂ (×3) after each additional charge. After 2 days total reaction time, the mixture was cooled to RT, poured into water (10 mL), and extracted with EtOAc (10 mL×3). The combined organic layers were dried over Na₂SO₄, filtered, and the filtrate concentrated under reduced pressure. The residue was purified by column chromatography (SiO₂, 0-5% MeOH/DCM gradient) to afford the title compound (95 mg, 47%) as a yellow oil. ES/MS m/z 961.8/963.3 (M+H), Cl isotope.

### Preparation 491

### Methyl (R)-3-(1-(2-(2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)benzoate

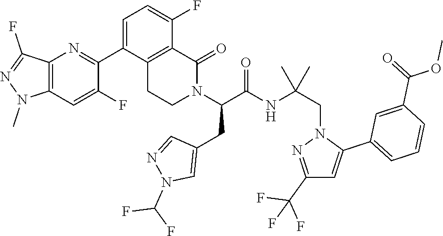 [SMILES: COC(=O)c1cccc(-c2cc(C(F)(F)F)nn2CC(C)(C)NC(=O)[C@@H](Cc2cnn(C(F)F)c2)N2CCc3c(-c4nc5c(F)nn(C)c5cc4F)ccc(F)c3C2=O)c1]

To a 1-dram vial were added (R)—N-(1-(5-bromo-3-(trifluoromethyl)-1H-pyrazol-1-yl)-2-methylpropan-2-yl)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamide (65.0 mg, 82.4 μmol), (3-(methoxycarbonyl)phenyl)boronic acid (18.6 mg, 103 μmol), [1,1′-bis(diphenylphosphino)ferrocene]dichloropalladium(II)·CH₂Cl₂ (6.6 mg, 8.1 μmol), and Cs₂CO₃ (98.5 mg, 302 μmol). 1,4-dioxane (850 μL) and water (210 μL) were added, and the resulting suspension was sparged with argon for 5 min. The reaction mixture was then heated to 90° C. under argon. After 1 h, the mixture was cooled to RT and was purified directly by column chromatography (SiO₂, 0-100% 3:1 EtOAc/EtOH in heptane gradient) to afford the title compound (71.8 mg, 93%) as a glassy solid. ES/MS m/z 844.4 (M+H).

### Preparation 492

### tert-Butyl (1-(5-(2-(2-hydroxypropan-2-yl)pyrimidin-5-yl)-3-(trifluoromethyl)-1H-pyrazol-1-yl)-2-methylpropan-2-yl)carbamate

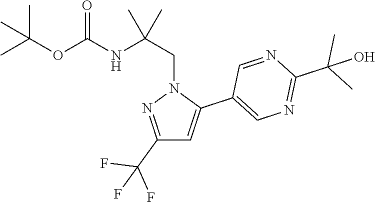 [SMILES: CC(C)(Cn1nc(C(F)(F)F)cc1-c1cnc(C(C)(C)O)nc1)NC(=O)OC(C)(C)C]

To tert-butyl (1-(5-(1,3,6,2-dioxazaborocan-2-yl)-3-(trifluoromethyl)-1H-pyrazol-1-yl)-2-methylpropan-2-yl)carbamate (24.00 g, 56.54 mmol) was added 2-(5-bromopyrimidin-2-yl)propan-2-ol (14.31 g, 62.63 mmol), 1 M Na₂CO₃ (aq.) (170 mL, 170 mmol), BrettPhos-Pd-G3 (5.21 g, 5.69 mmol), 1,4-dioxane (400 mL) and isopropanol (80 mL). The resulting mixture was evacuated and backfilled with N₂ (3×), stirred at 70° C. for 16 h, then filtered, rinsing with EtOAc (30 mL×3). The filtrate was concentrated under reduced pressure, then diluted with water (300 mL) and extracted with EtOAc (3 L×3). The combined organic extracts were dried over Na₂SO₄, filtered, and concentrated under reduced pressure. The residue was purified on silica gel, eluting with 0-20% EtOAc/hexanes to provide the title compound (19.6 g, 74%) as a yellow solid. ES-MS m/z=444.3 [M+H].

### Preparation 493

### 2-(5-(1-(2-Amino-2-methylpropyl)-3-(trifluoromethyl)-1H-pyrazol-5-yl)pyrimidin-2-yl)propan-2-ol

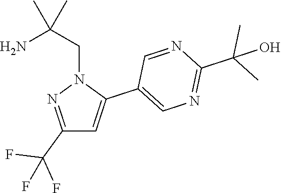 [SMILES: CC(C)(N)Cn1nc(C(F)(F)F)cc1-c1cnc(C(C)(C)O)nc1]

To a vial was added a solution of tert-butyl (1-(5-(2-(2-hydroxypropan-2-yl)pyrimidin-5-yl)-3-(trifluoromethyl)-1H-pyrazol-1-yl)-2-methylpropan-2-yl)carbamate (44.3 mg, 100 μmol) in DCM (1 mL). The vial was cooled in an ice bath, then TFA (150 μL, 1.95 mmol) was added and stirring was initiated. After 2.5 h, the reaction was diluted with additional DCM and quenched with sat. aq. Na₂CO₃. The organic layer was removed, and the aqueous layer was extracted an additional 3× with DCM. The combined organic layers were then dried over Na₂SO₄, filtered, and concentrated to afford the title compound (41.6 mg, 73% purity, 88% yield) as a yellow solid. ES-MS m/z 344.2 (M+H).

### Preparation 504

### 2-(5-(1-(2-Amino-2-methylpropyl)-3-(trifluoromethyl)-1H-pyrazol-5-yl)pyrimidin-2-yl)propan-2-ol hydrochloride

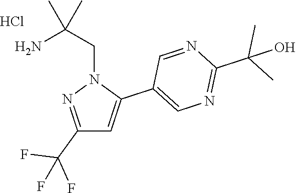 [SMILES: Cl ; CC(C)(N)Cn1nc(C(F)(F)F)cc1-c1cnc(C(C)(C)O)nc1]

To a mixture of tert-butyl (1-(5-(2-(2-hydroxypropan-2-yl)pyrimidin-5-yl)-3-(trifluoromethyl)-1H-pyrazol-1-yl)-2-methylpropan-2-yl)carbamate (3.45 g, 6.69 mmol) in 1,4-dioxane (15 mL) was added 2.0 M HCl in 1,4-dioxane (30 mL, 60 mmol). After stirring at 25° C. for 3 h, the reaction mixture was filtered, and the filter cake was washed with DCM (3×20 mL) and vacuum dried to afford a quantitative yield of the title compound (3.02 g, 85% purity) as a white solid. ES-MS m/z 344.1 (M+H).

### Preparation 494

### tert-Butyl (1-(5-(6-(2-hydroxypropan-2-yl)pyridin-3-yl)-3-(trifluoromethyl)-1H-pyrazol-1-yl)-2-methylpropan-2-yl)carbamate

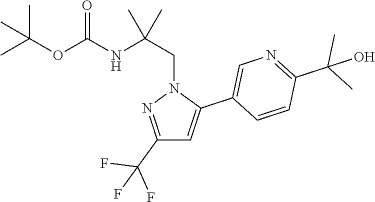 [SMILES: CC(C)(Cn1nc(C(F)(F)F)cc1-c1ccc(C(C)(C)O)nc1)NC(=O)OC(C)(C)C]

To a mixture of 2-(5-bromo-2-pyridyl)propan-2-ol (304 mg, 1.41 mmol), tert-butyl (1-(5-(1,3,6,2-dioxazaborocan-2-yl)-3-(trifluoromethyl)-1H-pyrazol-1-yl)-2-methylpropan-2-yl)carbamate (581 mg, 1.38 mmol) and Na₂CO₃ (1 M aq. solution, 4.0 mL, 4.0 mmol) in 1,4-dioxane (10 mL) and IPA (2.5 mL) was added BrettPhos-Pd-G3 (129 mg, 142 μmol). The mixture was degassed and refilled with N₂ 3 times and stirred at 70° C. for 16 h under N₂. The reaction mixture was quenched by the addition of H₂O (20 mL) and extracted with EtOAc (20 mL×2). The combined organic layers were washed with sat. aq. NaCl (20 mL×2), dried over Na₂SO₄, filtered and concentrated under reduced pressure. The residue was purified by normal phase chromatography (SiO₂, 0-50% EtOAc/hexanes gradient) to afford the title compound (431 mg, 69% yield) as a light yellow solid. ES-MS m/z 443.1 (M+H).

### Preparation 495

### tert-Butyl (1-(5-(2-(hydroxymethyl)pyrimidin-5-yl)-3-(trifluoromethyl)-1H-pyrazol-1-yl)-2-methylpropan-2-yl)carbamate

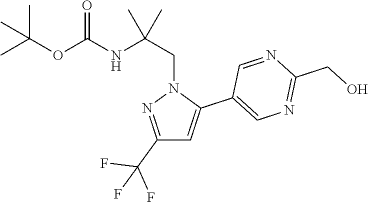 [SMILES: CC(C)(Cn1nc(C(F)(F)F)cc1-c1cnc(CO)nc1)NC(=O)OC(C)(C)C]

5-Bromopyrimidine-2-methanol (63.4 mg, 335 μmol), tert-butyl (1-(5-(1,3,6,2-dioxazaborocan-2-yl)-3-(trifluoromethyl)-1H-pyrazol-1-yl)-2-methylpropan-2-yl)carbamate (173.2 mg, 412.1 μmol), potassium phosphate, tribasic (187.1 mg, 881.5 μmol), BrettPhos-Pd-G3 (30.0 mg, 33.1 μmol), and a magnetic stir-bar were added to a 2-dram vial. Then 1,4-dioxane (2 mL), water (0.4 mL), and isopropanol (0.4 mL) were added and the resulting suspension was sparged with argon for ˜10 minutes. The vial cap was then replaced with one with an unpunctured septum under a flow of argon and the reaction was heated to 65° C. with stirring overnight. After sitting at room temperature for 1 day, the reaction was diluted with EtOAc and filtered through a plug of diatomaceous earth/Na₂SO₄, rinsing with EtOAc. The filtrate was concentrated and purified silica gel chromatography (0-100% EtOAc/heptane gradient) to afford the title compound (118.7 mg, 82%) as a light tan solid. ES-MS m/z 360.2 (M-tBu+H).

### Preparation 496

### 2-(5-(1-(2-Amino-2-methylpropyl)-3-(trifluoromethyl)-1H-pyrazol-5-yl)pyridin-2-yl)propan-2-ol trifluoromethanesulfonic acid salt

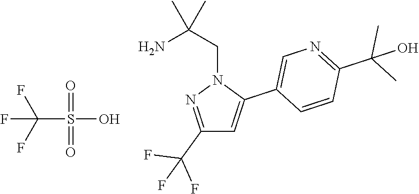 [SMILES: O=S(=O)(O)C(F)(F)F ; CC(C)(N)Cn1nc(C(F)(F)F)cc1-c1ccc(C(C)(C)O)nc1]

In a dried 100 mL round bottom flask, a solution of tert-butyl (1-(5-(6-(2-hydroxypropan-2-yl)pyridin-3-yl)-3-(trifluoromethyl)-1H-pyrazol-1-yl)-2-methylpropan-2-yl)carbamate (401 mg, 897 μmol) in DCM (10 mL) was treated with trimethylsilyl trifluoromethanesulfonate (300 μL, 1.65 mmol). The resulting mixture was stirred at 20° C. for 1 h then concentrated under reduced pressure. The yellow solid residue was stirred with hexanes (10 mL) at 20° C. for 15 min. The mixture was filtered, and the filter cake was washed with hexanes (3× 8 mL) and vacuum dried to obtain a quantitative yield of the title compound (593 mg, 74% purity) as a white solid. ES-MS m/z 343.2 (M+H).

### Preparation 497

### (5-(1-(2-Amino-2-methylpropyl)-3-(trifluoromethyl)-1H-pyrazol-5-yl)pyrimidin-2-yl)methanol hydrochloride

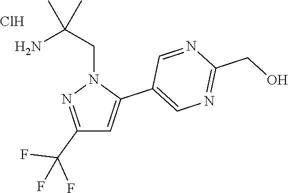 [SMILES: Cl ; CC(C)(N)Cn1nc(C(F)(F)F)cc1-c1cnc(CO)nc1]

Tert-butyl (1-(5-(2-(hydroxymethyl)pyrimidin-5-yl)-3-(trifluoromethyl)-1H-pyrazol-1-yl)-2-methylpropan-2-yl)carbamate (118.7 mg, 274.3 μmol) and a magnetic stir-bar were added to a scintillation vial and cooled to 0° C. Stirring was initiated, and then a solution of HCl (1.5 mL of a 4 M soln. in 1,4-dioxane, 6.0 mmol) was added. The reaction was allowed to continue for 2.1 h, then concentrated under reduced pressure to give the title compound (110 mg, 87% purity, 100% yield) as a foamy, off-white solid. ES-MS m/z 316.2 (M+H).

### Preparation 498

### Methyl 2-((tert-butoxycarbonyl)amino)-3-(1-(methyl-d3)-1H-pyrazol-4-yl)propanoate

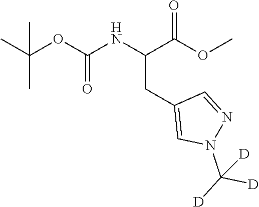 [SMILES: [2H]C([2H])([2H])n1cc(CC(NC(=O)OC(C)(C)C)C(=O)OC)cn1]

To a 2250 mL Parr bottle was added 10% Pd/C (8.35 g, 10% Wt, 7.85 mmol). The bottle was placed under a stream of N₂ and then MeOH (100 mL) was added followed by methyl (E)-2-((tert-butoxycarbonyl)amino)-3-(1-(methyl-d3)-1H-pyrazol-4-yl)acrylate (83.0 g, 292 mmol) as a solution in MeOH (740 mL). The bottle was placed on a Parr shaker, sealed, and purged with N₂ (5×) followed by H₂ (5×). The bottle was pressurized to an initial pressure of 50 psi with H₂ and heated to 50° C. for 5.5 h. The following day, the reaction mixture was filtered through a pad of diatomaceous earth, using additional MeOH to rinse the filter cake. The resulting filtrate was concentrated under reduced pressure to afford a colorless oil which solidified upon standing to afford the title compound (84.28 g, quantitative yield) as a white solid. ES-MS m/z 287.4 (M+H).

### Preparation 499

### 2-((tert-Butoxycarbonyl)amino)-3-(1-(methyl-d3)-1H-pyrazol-4-yl)propanoic acid

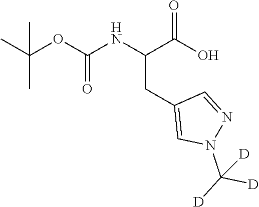 [SMILES: [2H]C([2H])([2H])n1cc(CC(NC(=O)OC(C)(C)C)C(=O)O)cn1]

A solution of methyl 2-((tert-butoxycarbonyl)amino)-3-(1-(methyl-d3)-1H-pyrazol-4-yl)propanoate (83.0 g, 281 mmol) in THF (830 mL) was cooled to 0° C. in an ice bath. A solution of lithium hydroxide (10.1 g, 421 mmol) in water (830 mL) was added slowly. The reaction mixture was allowed to warm to RT and stir overnight. The reaction mixture was concentrated to remove THF, extracted with EtOAc (2×300 mL), then the pH was adjusted to 3 with citric acid and the suspension was allowed to stir for 1 h. The precipitate was collected by vacuum filtration, rinsing with water to afford the title compound (68.32 g, 89.3% yield) as a white solid. ES-MS m/z 273.4 (M+H)+

### Preparation 500

### (R)-1-Phenylethan-1-amine (R)-2-((tert-butoxycarbonyl)amino)-3-(1-(methyl-d3)-1H-pyrazol-4-yl)propanoate

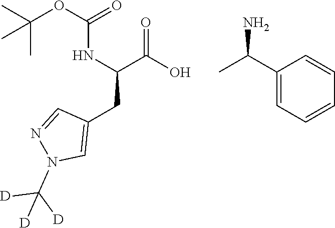

2-((tert-Butoxycarbonyl)amino)-3-(1-(methyl-d3)-1H-pyrazol-4-yl)propanoic acid (68.3 g, 251 mmol) was dissolved in methyl acetate (2.1 L) and R—(+)-alpha-phenylethylamine (35 mL, 270 mmol) was added dropwise via syringe. The reaction mixture was allowed to stir under N₂ overnight. The solid was collected via vacuum filtration, rinsing with additional methyl acetate to afford (R)-1-phenylethan-1-amine 2-((tert-butoxycarbonyl)amino)-3-(1-(methyl-d3)-1H-pyrazol-4-yl)propanoate (57.42 g, 125 mmol, 50.0%, 86.0% purity) as a white solid. A portion of the solid (48.95 g, 124.4 mmol) was re-dissolved in ACN (490 mL). The reaction mixture was heated to 90° C. for 2 h. The reaction mixture was allowed to cool back to RT, and the resulting solid was collected and dried by vacuum filtration over 2 h to afford the title compound as a white solid (44.26 g, 112.5 mmol, 90%, 96% ee). ES-MS m/z 273.2 [M+H].

### Preparation 501

### (R)-2-((tert-butoxycarbonyl)amino)-3-(1-(methyl-d3)-1H-pyrazol-4-yl)propanoic acid

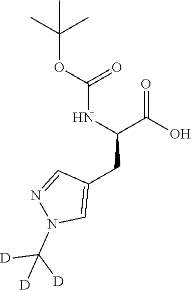 [SMILES: [2H]C([2H])([2H])n1cc(C[C@@H](NC(=O)OC(C)(C)C)C(=O)O)cn1]

(R)-1-Phenylethan-1-amine (R)-2-((tert-butoxycarbonyl)amino)-3-(1-(methyl-d3)-1H-pyrazol-4-yl)propanoate (15.0 g, 38.1 mmol) was suspended in water (50 mL) and EtOAc (100 mL). The reaction mixture was stirred vigorously at RT as aq HCl (3.18 mL, 12 molar, 38.1 mmol) was added dropwise. Upon complete addition, the mixture was transferred to a separatory funnel and the organic layer was collected. The aqueous layer was extracted with EtOAc (50 mL×2) and the combined organics were washed with sat. aq. NaCl (50 mL), dried over Na₂SO₄, and concentrated under reduced pressure to afford the title compound (12.02 g, 100%, 86% Purity) as a colorless foam. ES-MS m/z 273.2 [M+H]

### Preparation 502

### tert-Butyl (R)-(1-((1-(5-(2-(2-hydroxypropan-2-yl)pyrimidin-5-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)carbamate

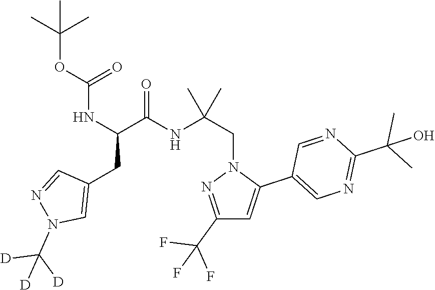 [SMILES: [2H]C([2H])([2H])n1cc(C[C@@H](NC(=O)OC(C)(C)C)C(=O)NC(C)(C)Cn2nc(C(F)(F)F)cc2-c2cnc(C(C)(C)O)nc2)cn1]

A vial containing (R)-2-((tert-butoxycarbonyl)amino)-3-(1-(methyl-d3)-1H-pyrazol-4-yl)propanoic acid (925 mg, 3.40 mmol), 2-(5-(1-(2-Amino-2-methylpropyl)-3-(trifluoromethyl)-1H-pyrazol-5-yl)pyrimidin-2-yl)propan-2-ol hydrochloride (1.23 g, 3.24 mmol), HATU (1.51 g, 3.97 mmol) was stirred on an ice bath as N,N-dimethylacetamide (12.5 mL) was added, followed by TEA (1.35 mL, 9.69 mmol). The reaction mixture was allowed to warm to RT and stirred overnight, then diluted with EtOAc (100 mL) and sat. aq. NaCl (75 mL). The aqueous layer was removed, and the organic layer was washed with sat. aq. NaCl (2×75 mL), sat. aq. NH₄Cl (75 mL), sat. aq. NaHCO₃ (75 mL) and sat. aq. NaCl (75 mL), dried over Na₂SO₄, filtered, concentrated, and purified by silica gel chromatography (gradient 0-100% 3:1 EtOAc:EtOH in heptane) to afford the title compound (1.77 g, 86%) as a foamy white solid. ES-MS m/z 598.6 (M+H).

### Preparation 503

### (R)-2-Amino-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide hydrochloride

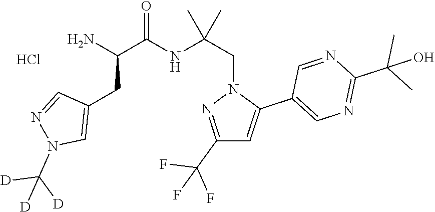 [SMILES: [2H]C([2H])([2H])n1cc(C[C@@H](N)C(=O)NC(C)(C)Cn2nc(C(F)(F)F)cc2-c2cnc(C(C)(C)O)nc2)cn1 ; Cl]

A solution of tert-butyl (R)-(1-((1-(5-(2-(2-hydroxypropan-2-yl)pyrimidin-5-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)carbamate (1.77 g, 2.78 mmol) in EtOH (11 mL) was cooled in an ice bath. HCl (4 M in dioxane) (5.5 mL, 22 mmol) was added. After 10 min, the ice bath was removed, and the mixture was stirred at room temperature for 3 h. The mixture was concentrated under vacuum, redissolved in EtOH (11 mL) and HCl (4 M in dioxane) (5.5 mL, 22 mmol) was added. The mixture was stirred for 2.5 h, then concentrated under vacuum. The residue was triturated with MTBE and filtered to provide the title compound (1.77 g, 75% purity, 89% yield) as a white solid. ES-MS m/z 498.4 (M+H).

### Preparation 505

### tert-Butyl (R)-(3-(1-(difluoromethyl)-1H-pyrazol-4-yl)-1-((1-(5-(2-(2-hydroxypropan-2-yl)pyrimidin-5-yl)-3-(trifluoromethyl)-1H-pyrazol-1-yl)-2-methylpropan-2-yl)amino)-1-oxopropan-2-yl)carbamate

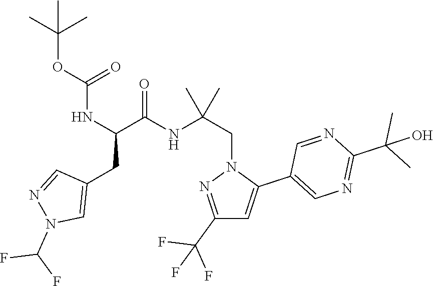 [SMILES: CC(C)(Cn1nc(C(F)(F)F)cc1-c1cnc(C(C)(C)O)nc1)NC(=O)[C@@H](Cc1cnn(C(F)F)c1)NC(=O)OC(C)(C)C]

To a mixture of (R)-2-((tert-butoxycarbonyl)amino)-3-(1-(difluoromethyl)-1H-pyrazol-4-yl)propanoic acid (2.06 g, 99% purity, 6.68 mmol) and 2-(5-(1-(2-amino-2-methylpropyl)-3-(trifluoromethyl)-1H-pyrazol-5-yl)pyrimidin-2-yl)propan-2-ol hydrochloride (3.00 g, 6.71 mmol) in DMA (30 mL) at 0° C. was added HATU (3.33 g, 8.67 mmol) and DIEA (3.60 mL, 20.5 mmol). After stirring at 0° C. for 1 h, the reaction mixture was diluted with EtOAc (70 mL) and washed with water (4×70 mL). The organic phase was dried over Na₂SO₄, filtered and concentrated under reduced pressure. The residue was purified by silica gel chromatography (0-46% EtOAc/hexanes gradient) to obtain the title compound (3.98 g, 89% yield) as a white solid. ES/MS m/z 631.3 (M+H).

### Preparation 506

### (R)-2-Amino-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide hydrochloride

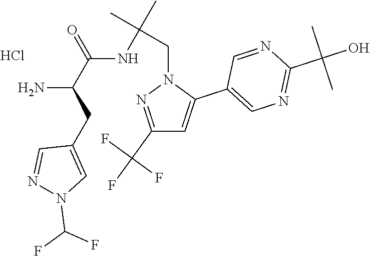 [SMILES: Cl ; CC(C)(Cn1nc(C(F)(F)F)cc1-c1cnc(C(C)(C)O)nc1)NC(=O)[C@H](N)Cc1cnn(C(F)F)c1]

To a mixture of tert-butyl (R)-(3-(1-(difluoromethyl)-1H-pyrazol-4-yl)-1-((1-(5-(2-(2-hydroxypropan-2-yl)pyrimidin-5-yl)-3-(trifluoromethyl)-1H-pyrazol-1-yl)-2-methylpropan-2-yl)amino)-1-oxopropan-2-yl)carbamate (3.98 g, 6.00 mmol) in 1,4-dioxane (10 mL) was added HCl (31.0 mL of a 2 M solution in 1,4-dioxane, 62.0 mmol). The reaction mixture was stirred at 24° C. for 3 h, concentrated under reduced pressure to afford the title compound (4.61 g, 75% purity, 100% yield) as a yellow solid. ES-MS m/z 531.1 (M+H).

### Preparation 507

### 2-(5-(Hydroxymethyl)-3-(trifluoromethyl)-1H-pyrazol-1-yl)-N-methylacetamide

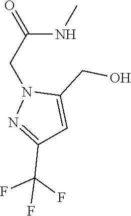 [SMILES: CNC(=O)Cn1nc(C(F)(F)F)cc1CO]

To a mixture of (3-(trifluoromethyl)-1H-pyrazol-5-yl)methanol (5.0 g, 30 mmol) and 2-bromo-N-methylacetamide (5.0 g, 33 mmol) in ACN (100 mL) was added Cs₂CO₃ (20 g, 60 mmol). The reaction mixture was stirred at 25° C. for 12 h and filtered, washing with EtOAc (30 mL×3). The filtrate was concentrated under reduced pressure and purified by silica gel chromatography (gradient 0-4% MeOH in CH₂Cl₂) to provide the title compound (5 g, 70%, 95% purity) as a white solid. ES-MS m/z 238.0 (M+H).

### Preparation 508

### 5-Methyl-2-(trifluoromethyl)-4,5-dihydropyrazolo[1,5-a]pyrazin-6 (7H)-one

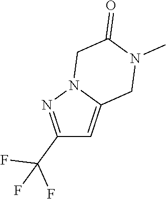

To a solution of 2-(5-(hydroxymethyl)-3-(trifluoromethyl)-1H-pyrazol-1-yl)-N-methylacetamide (5.0 g, 20 mmol) in toluene (100 mL) was added (cyanomethylene)tributylphosphorane (21 mL, 80 mmol) at 25° C. Then the reaction mixture was stirred at 100° C. for 2 h under N₂ atmosphere, then was diluted with H₂O (100 mL) and extracted with EtOAc (100 mL×2). The combined organic layers were washed with sat. aq. NaCl (60 mL×2), dried over Na₂SO₄, filtered, and concentrated. The crude product was purified by silica gel chromatography, eluting with 0-70% EtOAc in petroleum ether to give the title compound (3.1 g, 49%, 70% Purity) as a yellow solid. ES-MS m/z 220 (M+H).

### Preparation 509

### 2-Methyl-N-(2-(5-methyl-6-oxo-2-(trifluoromethyl)-4,5,6,7-tetrahydropyrazolo[1,5-a]pyrazin-7-yl)propan-2-yl)propane-2-sulfinamide

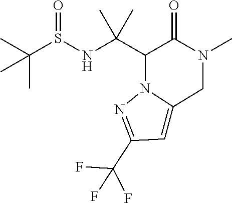 [SMILES: CN1Cc2cc(C(F)(F)F)nn2C(C(C)(C)NS(=O)C(C)(C)C)C1=O]

To a solution of 5-methyl-2-(trifluoromethyl)-4,5-dihydropyrazolo[1,5-a]pyrazin-6 (7H)-one (2.7 g, 70% Wt, 8.6 mmol) in THF (27 mL) was added LDA (2.0 M in THF, 8.6 mL, 2 molar, 17 mmol) at −60° C. The reaction was stirred at −60° C. for 15 min then, chlorotitanium triisopropoxide (1M in DCM, 8.6 mL, 1 molar, 8.6 mmol) was added and the reaction was stirred at −60° C. for 20 minutes followed by the addition of 2-methyl-N-(propan-2-ylidene)propane-2-sulfinamide (3.9 g, 22 mmol) in THF (3 mL). The reaction solution was stirred at −60° C. ˜25° C. for 12 h under N₂. The reaction mixture was quenched by the addition of H₂O (60 mL). The reaction was filtered to remove precipitate and the filtrate was washed with EtOAc (2×60 mL). The combined organic layer was washed with sat. aq. NaCl (2× 60 mL), dried over anhydrous Na₂SO₄, filtered and concentrated. The residue was purified by silica gel chromatography (0-100% EtOAc in petroleum ether) to afford the title compound in 2 batches (1.35 g, 80% purity, 33% yield and 1.7 g, 60% purity, 31% yield) both as yellow solids. ES-MS m/z 381.2 (M+H)

### Preparation 510

### 7-(2-Aminopropan-2-yl)-5-methyl-2-(trifluoromethyl)-4,5-dihydropyrazolo[1,5-a]pyrazin-6 (7H)-one (Isomer 1)

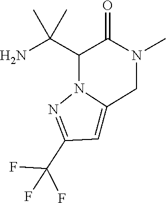 [SMILES: CN1Cc2cc(C(F)(F)F)nn2C(C(C)(C)N)C1=O]

To a solution of 2-methyl-N-(2-(5-methyl-6-oxo-2-(trifluoromethyl)-4,5,6,7-tetrahydropyrazolo[1,5-a]pyrazin-7-yl)propan-2-yl)propane-2-sulfinamide (3.05 g, 80% purity, 6.41 mmol) in 1,4-dioxane (10 mL) was added 2 M HCl in dioxane (16 mL, 32 mmol). The resulting mixture was stirred at 26° C. for 1 h then concentrated under reduced pressure. The residue was diluted with H₂O (10 mL) and extracted with DCM (2× 15 mL). The aqueous phase was concentrated, and the residue was diluted with DCM (40 mL). The resulting suspension was basified to pH=8 with aq. Na₂CO₃, dried over Na₂CO₃, filtered and concentrated under reduced pressure to give the title compound as a racemic mixture (1.6 g, 89% yield) as yellow oil. The racemate was subjected to chiral SFC chromatography [column: Daicel CHIRALPAK® AD (30 ×250 mm, 10 μm); mobile phase: isocratic 30% EtOH (with 0.1% NH₄OH): 70% CO₂; flow rate: 130 mL/min; 90 injections] to give the first eluting peak as the title compound (Isomer 1, 663 mg, 90% purity, 38% recovery, 97% e.e). ES-MS m/z 277.1 (M+H).

### Preparation 511

### (R)-3-Bromo-2-(2-chloroethyl)-N-(3-(1-(difluoromethyl)-1H-pyrazol-4-yl)-1-((1-(5-(2-(2-hydroxypropan-2-yl)pyrimidin-5-yl)-3-(trifluoromethyl)-1H-pyrazol-1-yl)-2-methylpropan-2-yl)amino)-1-oxopropan-2-yl)-6-fluorobenzamide

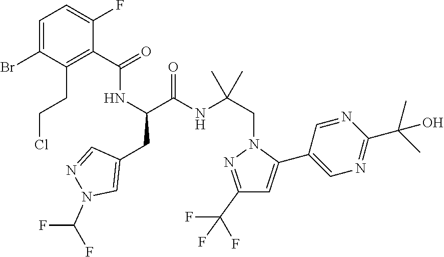 [SMILES: CC(C)(Cn1nc(C(F)(F)F)cc1-c1cnc(C(C)(C)O)nc1)NC(=O)[C@@H](Cc1cnn(C(F)F)c1)NC(=O)c1c(F)ccc(Br)c1CCCl]

To a mixture of (R)-2-amino-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide hydrochloride (4.61 g, 75% Wt, 6.10 mmol) and TEA (10.0 mL, 71.0 mmol) in THF (30 mL) was added a solution of 3-bromo-2-(2-chloroethyl)-6-fluorobenzoyl chloride (6.87 g, 45% Wt, 10.3 mmol) in THF (20 mL) at 0° C. The mixture was degassed and purged with N₂. The reaction mixture was stirred at 23° C. for 2 h under N₂. The reaction mixture was diluted with water (80 mL) and extracted with EtOAc (80 mL×3). The combined organic layers were dried over anhydrous Na₂SO₄, filtered, and concentrated under reduced pressure. The residue was purified by silica gel chromatography (0-3% MeOH/DCM gradient) to give the title compound (5.01 g, 78% purity, 81% yield) as a yellow solid. ES/MS m/z 795.1 (M+H).

### Preparation 512

### (R)-2-(5-Bromo-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

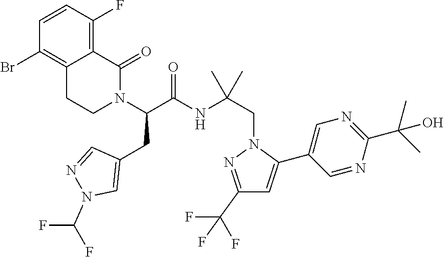 [SMILES: CC(C)(Cn1nc(C(F)(F)F)cc1-c1cnc(C(C)(C)O)nc1)NC(=O)[C@@H](Cc1cnn(C(F)F)c1)N1CCc2c(Br)ccc(F)c2C1=O]

To (R)-3-bromo-2-(2-chloroethyl)-N-(3-(1-(difluoromethyl)-1H-pyrazol-4-yl)-1-((1-(5-(2-(2-hydroxypropan-2-yl)pyrimidin-5-yl)-3-(trifluoromethyl)-1H-pyrazol-1-yl)-2-methylpropan-2-yl)amino)-1-oxopropan-2-yl)-6-fluorobenzamide (5.01 g, 78% purity, 4.92 mmol) in ACN (50 mL) was added Cs₂CO₃ (2.40 g, 7.29 mmol) at 24° C., then the reaction mixture was stirred at 24° C. for 12 h. The reaction mixture was diluted with water (80 mL) and extracted with EtOAc (80 mL×3). The combined organic layers were dried over anhydrous Na₂SO₄, filtered and concentrated under reduced pressure. The residue was dissolved in ACN (50 mL) and 1 N HCl (aq.) (5 mL), and the mixture was sonicated at 22° C. for 10 min. The mixture was diluted with water (50 mL) and extracted with EtOAc (60 mL×3). The combined organic layers were dried over anhydrous Na₂SO₄, filtered and concentrated under reduced pressure. The residue was purified by silica gel chromatography (0-75% ethyl acetate/hexanes gradient) to give the title compound (3.80 g, 89% purity, 91% yield) as a white solid. ES/MS m/z 757.3 (M+H).

### Preparation 513

### (R)-3-(1-(Difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

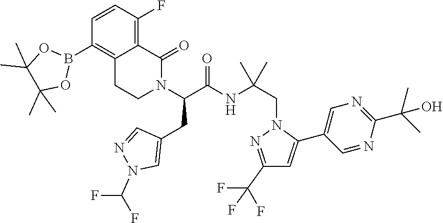 [SMILES: CC(C)(Cn1nc(C(F)(F)F)cc1-c1cnc(C(C)(C)O)nc1)NC(=O)[C@@H](Cc1cnn(C(F)F)c1)N1CCc2c(B3OC(C)(C)C(C)(C)O3)ccc(F)c2C1=O]

To a (R)-2-(5-bromo-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide (2.40 g, 89% Wt, 2.82 mmol) in 1,4-dioxane (30 mL) was added potassium acetate (842 mg, 8.49 mmol), bis(pinacolato)diborane (7.25 g, 28.3 mmol), 4 Å molecular sieves (4.90 g), and Pd(dppf)Cl₂·CH₂Cl₂ (240 mg, 291 μmol) at RT, then the mixture reaction was degassed and purged with N₂. The mixture was stirred at 80° C. for 16 h under N₂ atmosphere, then filtered and the filter cake was washed with EtOAc (30 mL×3). The filtrate was concentrated under reduced pressure and the residue was purified by silica gel chromatography (0-2% MeOH/DCM gradient) to give the title compound (3.29 g, 71% purity, 100% yield) as a yellow oil. ES/MS m/z 805.3 (M+H).

### Preparation 514

### methyl (R)-2-(5-(6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoate

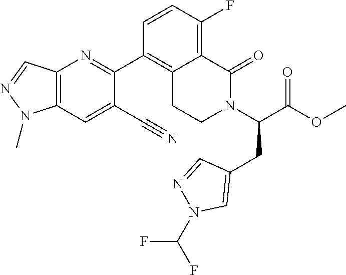 [SMILES: COC(=O)[C@@H](Cc1cnn(C(F)F)c1)N1CCc2c(-c3nc4cnn(C)c4cc3C#N)ccc(F)c2C1=O]

The title compound was prepared in a similar manner as described in Preparation 204 using 5-chloro-1-methyl-1H-pyrazolo[4,3-b]pyridine-6-carbonitrile (95 mg, 0.45 mmol) and (R)-(2-(3-(1-(difluoromethyl)-1H-pyrazol-4-yl)-1-methoxy-1-oxopropan-2-yl)-8-fluoro-1-oxo-1,2,3,4-tetrahydroisoquinolin-5-yl)boronic acid. ES-MS m/z 524.4 (M+H).

### Preparation 515

### (R)-2-(5-(6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid

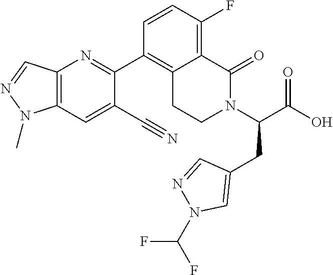 [SMILES: Cn1ncc2nc(-c3ccc(F)c4c3CCN([C@H](Cc3cnn(C(F)F)c3)C(=O)O)C4=O)c(C#N)cc21]

The title compound was prepared in a similar manner as described in Preparation 205 using methyl (R)-2-(5-(6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoate. ES-MS m/z 510.3 (M+H).

### Preparation 516

### (R)-3-bromo-2-(2-chloroethyl)-6-fluoro-N-(1-((1-(5-(2-(2-hydroxypropan-2-yl)pyrimidin-5-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)benzamide

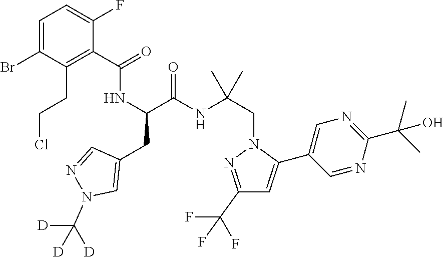 [SMILES: [2H]C([2H])([2H])n1cc(C[C@@H](NC(=O)c2c(F)ccc(Br)c2CCCl)C(=O)NC(C)(C)Cn2nc(C(F)(F)F)cc2-c2cnc(C(C)(C)O)nc2)cn1]

The title compound was prepared in a similar manner as described in Preparation 511 using 3-bromo-2-(2-chloroethyl)-6-fluorobenzoyl chloride (337 mg, 0.97 mmol) and (R)-2-amino-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide hydrochloride using DIEA in place of TEA. ES-MS m/z 760.4 (M+H).

### Preparation 517

### (R)-2-(5-bromo-8-fluoro-1-oxo-3,4-dihydroisoquinolin-2 (1H)-yl)-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide

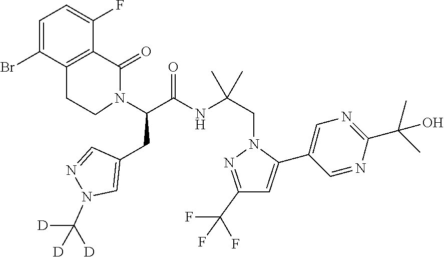 [SMILES: [2H]C([2H])([2H])n1cc(C[C@H](C(=O)NC(C)(C)Cn2nc(C(F)(F)F)cc2-c2cnc(C(C)(C)O)nc2)N2CCc3c(Br)ccc(F)c3C2=O)cn1]

The title compound was prepared in a similar manner as described in Preparation 512 using (R)-3-bromo-2-(2-chloroethyl)-6-fluoro-N-(1-((1-(5-(2-(2-hydroxypropan-2-yl)pyrimidin-5-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)benzamide. ES-MS m/z 726.4 (M+H).

### EXAMPLES

### Example 1

### (R)-3-(1-(Difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

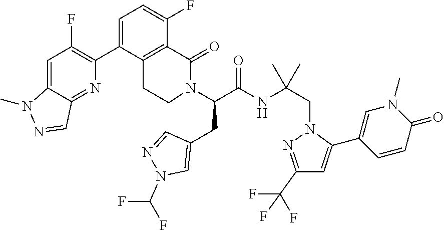 [SMILES: Cn1cc(-c2cc(C(F)(F)F)nn2CC(C)(C)NC(=O)[C@@H](Cc2cnn(C(F)F)c2)N2CCc3c(-c4nc5cnn(C)c5cc4F)ccc(F)c3C2=O)ccc1=O]

To a 20 mL vial with stir bar, nitrogen needle inlet with septum was added: (R)-3-(1-(difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (1.00 g, 1.29 mmol), 5-chloro-6-fluoro-1-methyl-1H-pyrazolo[4,3-b]pyridine (287 mg, 1.55 mmol), cesium carbonate (840 mg, 2.58 mmol) and Pd(dppf)Cl₂ (142 mg, 194 μmol) followed by 1,4-dioxane (7.81 mL) and water (781 μL). The mixture was vacuum degassed and purged with N₂ (3×), heated to 70° C. overnight then diluted with EtOAc and washed with water. The organic phase was concentrated under reduced pressure. The residue was purified by reversed phase C18 flash chromatography, eluting with 30-60% ACN/10 mM aq. NH₄HCO₃, pH=10 (with 5% MeOH), to obtain the title compound (740 mg, 72%) as a light grey solid. ES-MS m/z 799.6 (M+H).

### Example 2

### (R)-3-(1-(Difluoromethyl)-1H-pyrazol-4-yl)-2-(8-fluoro-5-(5-fluoro-1-methyl-1H-pyrazolo[3,4-b]pyridin-6-yl)-1-oxo-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

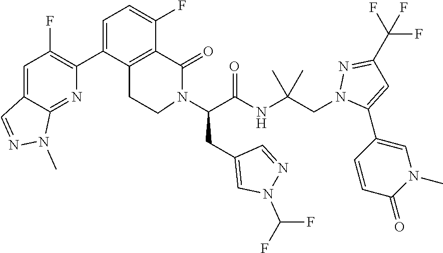 [SMILES: Cn1cc(-c2cc(C(F)(F)F)nn2CC(C)(C)NC(=O)[C@@H](Cc2cnn(C(F)F)c2)N2CCc3c(-c4nc5c(cnn5C)cc4F)ccc(F)c3C2=O)ccc1=O]

A 20-mL microwave vial containing a stir bar was charged with 6-chloro-5-fluoro-1-methyl-1H-pyrazolo[3,4-b]pyridine (60 mg, 0.32 mmol), (R)-3-(1-(difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (250 mg, 322 μmol), Pd (dppf) Cl₂·CH₂Cl₂ (26 mg, 32 μmol), and cesium carbonate (315 mg, 967 μmol). The vial was sealed with a cap, then evacuated and backfilled three times with N₂. A solution of 4:1 1,4-dioxane/water (3 mL) pre-sparged with nitrogen for 5 min was then added to the vial. The reaction mixture was stirred at 90° C. for 2 h. The reaction mixture was allowed to cool to RT, filtered, then the filtrate was concentrated under reduced pressure. The residue was purified by reversed phase C18 flash chromatography, eluting with 45-60% ACN/10 mM aq. NH₄HCO₃, pH=10 (with 5% MeOH), to obtain the title compound (152.2 mg, 59%) as a white solid. ES-MS m/z 799.2 (M+H).

The compounds in the following table were prepared in similar manner as described in Example 2 using the appropriate aryl halide and boronic ester prepared previously. Reactants can be added in different orders or in differing equivalency, solvents can be differing ratios and mixtures of ethers and alcohols, reactions can be run at 70-90° C. for 1-24 h, and purification methods were adjusted to suit the compounds. Such variances would be apparent to one skilled in the art.

TABLE 45

| | | | ES-MS |
|---|---|---|---|
| Example | Chemical Name | Structure | m/z |
| 3 | (R)-2-(5-(6-Cyano-1-(4- methoxybenzyl)-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(2- methyl-1-(5-(1-methyl-6- oxo-1,6-dihydropyridin-3- yl)-3-(trifluoromethyl)- 1H-pyrazol-1-yl)propan-2- yl)propanamide | 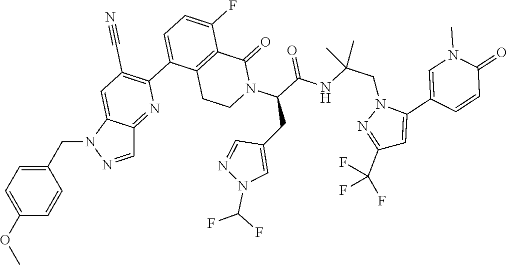 [SMILES: COc1ccc(Cn2ncc3nc(-c4ccc(F)c5c4CCN([C@H](Cc4cnn(C(F)F)c4)C(=O)NC(C)(C)Cn4nc(C(F)(F)F)cc4-c4ccc(=O)n(C)c4)C5=O)c(C#N)cc32)cc1] | 912.8 (M + H) |
| 4 | (R)-2-(5-(2-Cyano-4-(2- methyl-2H-pyrazolo[3,4- b]pyridin-5-yl)phenyl)-8- fluoro-1-oxo-3,4- dihydroisoquinolin-2(1H)- yl)-3-(1-(difluoromethyl)- 1H-pyrazol-4-yl)-N-(2- methyl-1-(5-(1-methyl-6- oxo-1,6-dihydropyridin-3- yl)-3-(trifluoromethyl)- 1H-pyrazol-1-yl)propan-2- yl)propanamide | 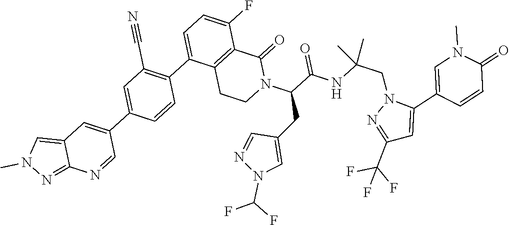 [SMILES: Cn1cc2cc(-c3ccc(-c4ccc(F)c5c4CCN([C@H](Cc4cnn(C(F)F)c4)C(=O)NC(C)(C)Cn4nc(C(F)(F)F)cc4-c4ccc(=O)n(C)c4)C5=O)c(C#N)c3)cnc2n1] | 882.2 (M + H) |
| 5 | (R)-2-(5-(5-Cyano-1- methyl-1H-pyrazolo[3,4- b]pyridin-6-yl)-8-fluoro-1- oxo-3,4- dihydroisoquinolin-2(1H)- yl)-3-(1-(difluoromethyl)- 1H-pyrazol-4-yl)-N-(2- methyl-1-(5-(1-methyl-6- oxo-1,6-dihydropyridin-3- yl)-3-(trifluoromethyl)- 1H-pyrazol-1-yl)propan-2- yl)propanamide | 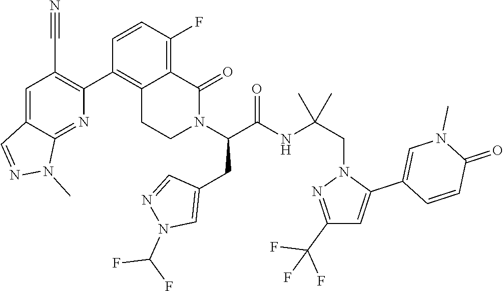 [SMILES: Cn1cc(-c2cc(C(F)(F)F)nn2CC(C)(C)NC(=O)[C@@H](Cc2cnn(C(F)F)c2)N2CCc3c(-c4nc5c(cnn5C)cc4C#N)ccc(F)c3C2=O)ccc1=O] | 806.2 (M + H) |
| 6ª | (R)-2-(5-(6-Cyano-1- methyl-1H-indazol-5-yl)- 8-fluoro-1-oxo-3,4- dihydroisoquinolin-2(1H)- yl)-3-(1-(difluoromethyl)- 1H-pyrazol-4-yl)-N-(2- methyl-1-(5-(1-methyl-6- oxo-1,6-dihydropyridin-3- yl)-3-(trifluoromethyl)- 1H-pyrazol-1-yl)propan-2- yl)propanamide | 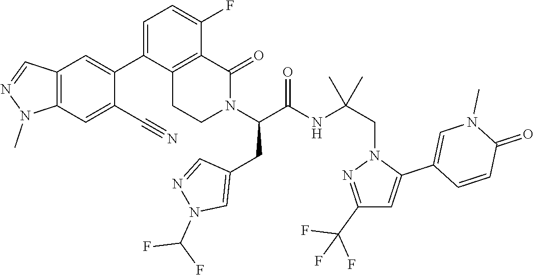 [SMILES: Cn1cc(-c2cc(C(F)(F)F)nn2CC(C)(C)NC(=O)[C@@H](Cc2cnn(C(F)F)c2)N2CCc3c(-c4cc5cnn(C)c5cc4C#N)ccc(F)c3C2=O)ccc1=O] | 805.2 (M + H) |
| 7 | (R)-3-(1- (Difluoromethyl)-1H- pyrazol-4-yl)-N-(1-(5-(5- fluoro-1-methyl-6-oxo- 1,6-dihydropyridin-3-yl)- 3-(trifluoromethyl)-1H- pyrazol-1-yl)-2- methylpropan-2-yl)-2-(8- fluoro-5-(5-fluoro-1- methyl-1H-pyrazolo[3,4- b]pyridin-6-yl)-1-oxo-3,4- dihydroisoquinolin-2(1H)- yl)propanamide | 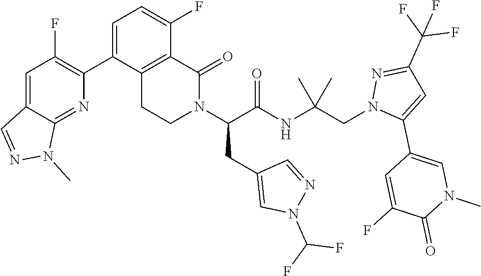 [SMILES: Cn1cc(-c2cc(C(F)(F)F)nn2CC(C)(C)NC(=O)[C@@H](Cc2cnn(C(F)F)c2)N2CCc3c(-c4nc5c(cnn5C)cc4F)ccc(F)c3C2=O)cc(F)c1=O] | 817.2 (M + H) |
| 8 | (R)-3-(1- (Difluoromethyl)-1H- pyrazol-4-yl)-N-(1-(5-(5- fluoro-1-methyl-6-oxo- 1,6-dihydropyridin-3-yl)- 3-(trifluoromethyl)-1H- pyrazol-1-yl)-2- methylpropan-2-yl)-2-(8- fluoro-5-(3- methylbenzo[d]isoxazol- 5-yl)-1-oxo-3,4- dihydroisoquinolin-2(1H)- yl)propanamide | 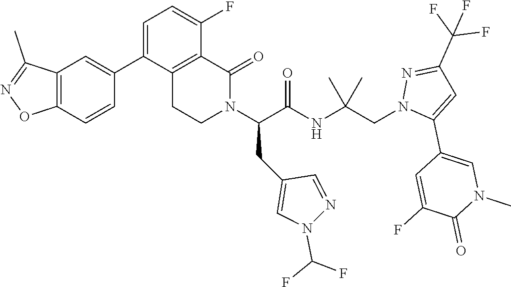 [SMILES: Cc1noc2ccc(-c3ccc(F)c4c3CCN([C@H](Cc3cnn(C(F)F)c3)C(=O)NC(C)(C)Cn3nc(C(F)(F)F)cc3-c3cc(F)c(=O)n(C)c3)C4=O)cc12] | 799.2 (M + H) |
| 9^{a} | (R)-3-(1- (Difluoromethyl)-1H- pyrazol-4-yl)-N-(1-(5-(5- fluoro-1-methyl-6-oxo- 1,6-dihydropyridin-3-yl)- 3-(trifluoromethyl)-1H- pyrazol-1-yl)-2- methylpropan-2-yl)-2-(8- fluoro-5-(6-fluoro-1- methyl-1H- benzo[d][1,2,3]triazol-5- yl)-1-oxo-3,4- dihydroisoquinolin-2(1H)- yl)propanamide | 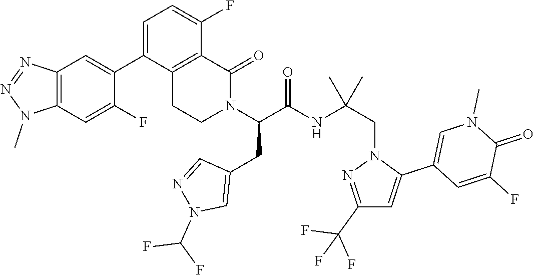 [SMILES: Cn1cc(-c2cc(C(F)(F)F)nn2CC(C)(C)NC(=O)[C@@H](Cc2cnn(C(F)F)c2)N2CCc3c(-c4cc5nnn(C)c5cc4F)ccc(F)c3C2=O)cc(F)c1=O] | 817.2 (M + H) |
| 10 | (R)-3-(1- (Difluoromethyl)-1H- pyrazol-4-yl)-N-(1-(5-(5- fluoro-1-methyl-6-oxo- 1,6-dihydropyridin-3-yl)- 3-(trifluoromethyl)-1H- pyrazol-1-yl)-2- methylpropan-2-yl)-2-(8- fluoro-5-(6-fluoro-1- methyl-1H-pyrazolo[4,3- b]pyridin-5-yl)-1-oxo-3,4- dihydroisoquinolin-2(1H)- yl)propanamide | 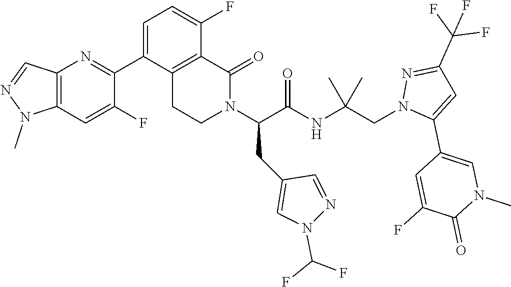 [SMILES: Cn1cc(-c2cc(C(F)(F)F)nn2CC(C)(C)NC(=O)[C@@H](Cc2cnn(C(F)F)c2)N2CCc3c(-c4nc5cnn(C)c5cc4F)ccc(F)c3C2=O)cc(F)c1=O] | 817.2 (M + H) |
| 11 | (R)-2-(5-(5-Cyano-1,3- dimethyl-1H- pyrazolo[3,4-b]pyridin-6- yl)-8-fluoro-1-oxo-3,4- dihydroisoquinolin-2(1H)- yl)-3-(1-(difluoromethyl)- 1H-pyrazol-4-yl)-N-(1-(5- (5-fluoro-1-methyl-6-oxo- 1,6-dihydropyridin-3-yl)- 3-(trifluoromethyl)-1H- pyrazol-1-yl)-2- methylpropan-2- yl)propanamide | 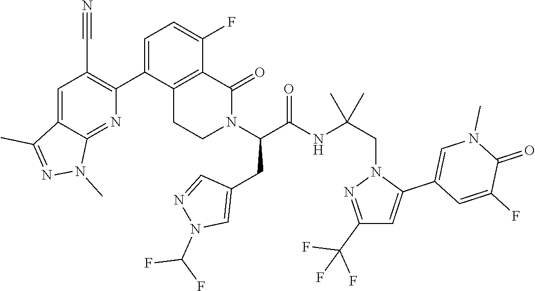 [SMILES: Cc1nn(C)c2nc(-c3ccc(F)c4c3CCN([C@H](Cc3cnn(C(F)F)c3)C(=O)NC(C)(C)Cn3nc(C(F)(F)F)cc3-c3cc(F)c(=O)n(C)c3)C4=O)c(C#N)cc12] | 838.2 (M + H) |
| 12^{a} | (R)-2-(5-(6-Cyano-1- methyl-1H-indazol-5-yl)- 8-fluoro-1-oxo-3,4- dihydroisoquinolin-2(1H)- yl)-N-(2-methyl-1-(5-(1- methyl-6-oxo-1,6- dihydropyridin-3-yl)-3- (trifluoromethyl)-1H- pyrazol-1-yl)propan-2-yl)- 3-(1-methyl-1H-pyrazol- 4-yl)propanamide | 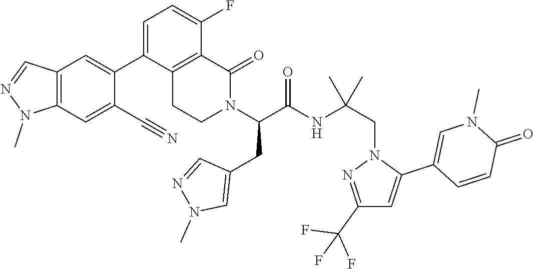 [SMILES: Cn1cc(C[C@H](C(=O)NC(C)(C)Cn2nc(C(F)(F)F)cc2-c2ccc(=O)n(C)c2)N2CCc3c(-c4cc5cnn(C)c5cc4C#N)ccc(F)c3C2=O)cn1] | 769.2 (M + H) |
| 13 | (R)-2-(5-(6-Cyano-1H- indazol-5-yl)-1-oxo-3,4- dihydroisoquinolin-2(1H)- yl)-N-(1-(4-fluorophenyl)- 2-methylpropan-2-yl)-3- (1-methyl-1H-pyrazol-4- yl)propanamide | 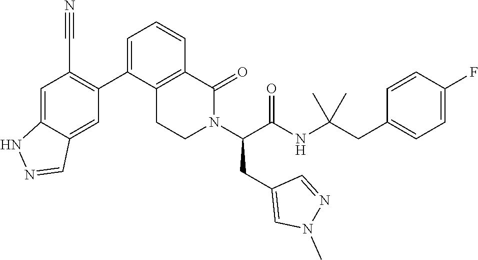 [SMILES: Cn1cc(C[C@H](C(=O)NC(C)(C)Cc2ccc(F)cc2)N2CCc3c(cccc3-c3cc4cn[nH]c4cc3C#N)C2=O)cn1] | 590.0 (M + H) |

^{a}XPhos Pd G4 (0.2 equiv) was used as catalyst; sodium carbonate (2.0 equiv) was used as base; and 1,4-dioxane/EtOH/water (10:5:1) used as solvent; and the reaction was heated at 90° C. for 16 h.

### Example 14

### (R)-2-(5-(6-Cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

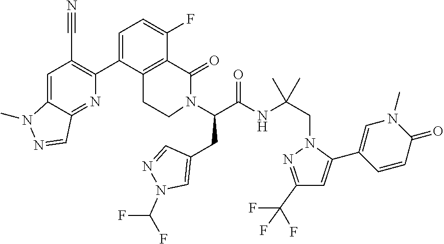 [SMILES: Cn1cc(-c2cc(C(F)(F)F)nn2CC(C)(C)NC(=O)[C@@H](Cc2cnn(C(F)F)c2)N2CCc3c(-c4nc5cnn(C)c5cc4C#N)ccc(F)c3C2=O)ccc1=O]

To a mixture of (R)-(2-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-8-fluoro-1-oxo-1,2,3,4-tetrahydroisoquinolin-5-yl)boronic acid (240 mg, 0.325 mmol) in 1,4-dioxane (5 mL), EtOH (5 mL) and water (0.5 mL) was added 5-chloro-1-methyl-1H-pyrazolo[4,3-b]pyridine-6-carbonitrile (71.8 mg, 0.358 mmol), sodium carbonate (103 mg, 0.976 mmol) and XPhos Pd G3 (55.1 mg, 0.065 mmol). The mixture was purged with N₂ and heated to 90° C. overnight. The reaction was cooled to RT, filtered and concentrated under reduced pressure. The residue was purified on silica gel, eluting with 0-4% MeOH in DCM to give the title compound as a white solid (106.9 mg, 9%). ES-MS m/z 806.3 (M+H).

The compounds in the following table were prepared in similar manner as described in Example 14 using the appropriate aryl halide and boronic acid prepared previously. Reactants can be added in different orders or in differing equivalency, and purification methods were adjusted to suit the compounds. Such variances would be apparent to one skilled in the art.

TABLE 46

| | | | ES-MS |
|---|---|---|---|
| Example | Chemical Name | Structure | m/z |
| 15 | (R)-2-(5-(6-Cyano-1- methyl-1H- benzo[d][1,2,3]triazol-5- yl)-8-fluoro-1-oxo-3,4- dihydroisoquinolin-2(1H)- yl)-3-(1-(difluoromethyl)- 1H-pyrazol-4-yl)-N-(2- methyl-1-(5-(1-methyl-6- oxo-1,6-dihydropyridin-3- yl)-3-(trifluoromethyl)- 1H-pyrazol-1-yl)propan-2- yl)propanamide | 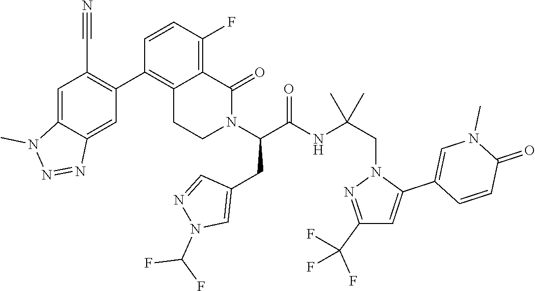 [SMILES: Cn1cc(-c2cc(C(F)(F)F)nn2CC(C)(C)NC(=O)[C@@H](Cc2cnn(C(F)F)c2)N2CCc3c(-c4cc5nnn(C)c5cc4C#N)ccc(F)c3C2=O)ccc1=O] | 806.2 (M + H) |
| 16 | (R)-2-(5-(5-Cyano-1- methyl-1H- benzo[d][1,2,3]triazol-6- yl)-8-fluoro-1-oxo-3,4- dihydroisoquinolin-2(1H)- yl)-3-(1-(difluoromethyl)- 1H-pyrazol-4-yl)-N-(2- methyl-1-(5-(1-methyl-6- oxo-1,6-dihydropyridin-3- yl)-3-(trifluoromethyl)- 1H-pyrazol-1-yl)propan-2- yl)propanamide | 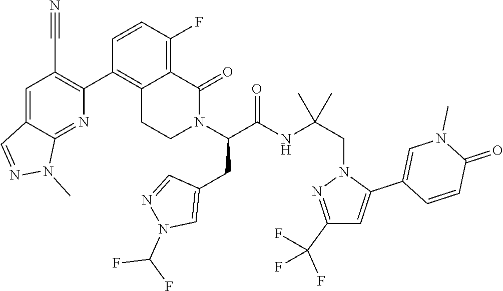 [SMILES: Cn1cc(-c2cc(C(F)(F)F)nn2CC(C)(C)NC(=O)[C@@H](Cc2cnn(C(F)F)c2)N2CCc3c(-c4nc5c(cnn5C)cc4C#N)ccc(F)c3C2=O)ccc1=O] | 806.2 (M + H) |
| 17^{a} | (R)-2-(5-(6-Cyano-1- methyl-1H-indazol-5-yl)- 8-fluoro-1-oxo-3,4- dihydroisoquinolin-2(1H)- yl)-3-(1-(difluoromethyl)- 1H-pyrazol-4-yl)-N-(1-(5- (5-fluoro-1-methyl-6-oxo- 1,6-dihydropyridin-3-yl)- 3-(trifluoromethyl)-1H- pyrazol-1-yl)-2- methylpropan-2- yl)propanamide | 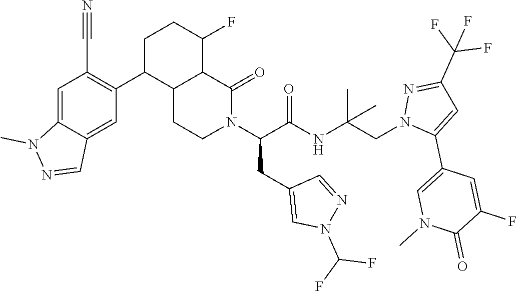 | 823.2 (M + H) |
| 18 | (R)-2-(5-(6-Cyano-1- methyl-1H-pyrazolo[4,3- b]pridin-5-yl)-8-fluoro-1- oxo-3,4- dihydroisoquinolin-2(1H)- yl)-N-(2-methyl-1-(5-(1- methyl-6-oxo-1,6- dihydropyridin-3-yl)-3- (trifluoromethyl)-1H- pyrazol-1-yl)propan-2-yl)- 3-(1-methyl-1H-pyrazol- 4-yl)propanamide | 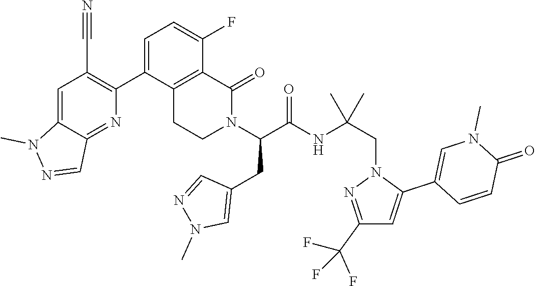 [SMILES: Cn1cc(C[C@H](C(=O)NC(C)(C)Cn2nc(C(F)(F)F)cc2-c2ccc(=O)n(C)c2)N2CCc3c(-c4nc5cnn(C)c5cc4C#N)ccc(F)c3C2=O)cn1] | 770.3 (M + H) |
| 19 | (R)-N-(1-(5-(1H-1,2,3- Triazol-5-yl)-3- (trifluoromethyl)-1H- pyrazol-1-yl)-2- methylpropan-2-yl)-2-(5- (6-cyano-1-methyl-1H- indazol-5-yl)-8-fluoro-1- oxo-3,4- dihydroisoquinolin-2(1H)- yl)-3-(1-methyl-1H- pyrazol-4-yl)propanamide | 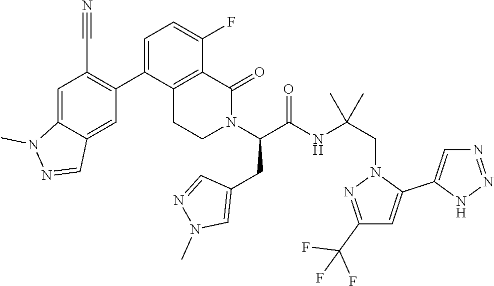 [SMILES: Cn1cc(C[C@H](C(=O)NC(C)(C)Cn2nc(C(F)(F)F)cc2-c2cnn[nH]2)N2CCc3c(-c4cc5cnn(C)c5cc4C#N)ccc(F)c3C2=O)cn1] | 729.2 (M + H) |
| 20^{b} | (R)-2-(5-(6-Cyano-3- methyl-2-oxo-2,3-dihydro- 1H-imidazo[4,5-b]pyridin- 5-yl)-8-fluoro-1-oxo-3,4- dihydroisoquinolin-2(1H)- yl)-N-(1-(5-fluoropyridin- 2-yl)-2-methylpropan-2- yl)-3-(1-methyl-1H- pyrazol-4-yl)propanamide | 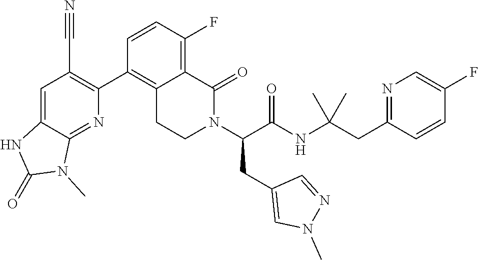 [SMILES: Cn1cc(C[C@H](C(=O)NC(C)(C)Cc2ccc(F)cn2)N2CCc3c(-c4nc5c(cc4C#N)[nH]c(=O)n5C)ccc(F)c3C2=O)cn1] | 640.7 (M + H) |

^{a}Pd(dppf)Cl₂ (0.14 equiv) used as catalyst; Cs₂CO₃ (3 equiv) used as base; 1,4-dioxane/water (4:1) used as solvent; heated at 90° C. for 1 h.
^{b}cataCXium ® A Pd G3 (0.1 equiv) used as catalyst; K₂CO₃ (3 equiv) used as base; 1,4-dioxane/water used as solvent; heated at 100° C. for 16 h.

### Example 21

### (R)-2-(5-(6-Cyano-1H-pyrazolo[4,3-b]pyridin-5-yl)-8-fluoro-1-oxo-3,4-dihydroisoquinolin-2 (1H)-yl)-N-(1-(5-fluoropyridin-2-yl)-2-methylpropan-2-yl)-3-(1-methyl-1H-pyrazol-4-yl)propanamide

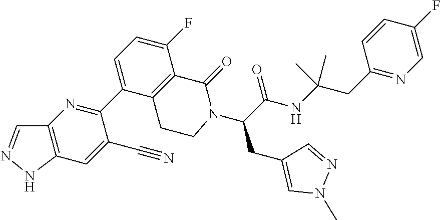 [SMILES: Cn1cc(C[C@H](C(=O)NC(C)(C)Cc2ccc(F)cn2)N2CCc3c(-c4nc5cn[nH]c5cc4C#N)ccc(F)c3C2=O)cn1]

To a solution of 1-acetyl-5-chloro-1H-pyrazolo[4,3-b]pyridine-6-carbonitrile (60 mg, 0.27 mmol) and (R)-(8-fluoro-2-(1-((1-(5-fluoropyridin-2-yl)-2-methylpropan-2-yl)amino)-3-(1-methyl-1H-pyrazol-4-yl)-1-oxopropan-2-yl)-1-oxo-1,2,3,4-tetrahydroisoquinolin-5-yl)boronic acid (0.15 g, 93% purity, 0.27 mmol) in a mixture of 1,4-dioxane (3 mL), EtOH (3 mL), and water (0.3 mL) were added Na₂CO₃ (58 mg, 0.54 mmol) and Pd(dppf)Cl₂ (40 mg, 54 μmol) at RT. The reaction mixture was then stirred at 90° C. for 16 h under N₂. After cooling to RT, an additional portion of Pd(dppf)Cl₂ (40 mg, 54 μmol) was added and the reaction mixture was re-purged with N₂. Stirring was continued at 90° C. for 8 h. The reaction mixture was filtered, rinsing with EtOAc (10 mL×2). The combined organic layers were washed with water (2×) and sat. aq. NaCl, dried over sodium sulfate, filtered, and the filtrate concentrated. The residue was first purified on silica gel, eluting with 0-15% MeOH in DCM to give a brown oil. The material was further purified by prep-TLC (DCM/MeOH 10:1) to give a brown oil of 85% purity. The material was subjected to a final purification by reverse phase HPLC (column: Welch Xtimate® C18, 200×40 mm, 7 mm) eluting with 10%-50% ACN in water (with 0.1% FA) over 25 min, to obtain the title compound (12.1 mg, 7%) as a white solid. ES-MS m/z 610.3 (M+H).

### Example 22

### (R)-3-(1-(Difluoromethyl)-1H-pyrazol-4-yl)-2-(8-fluoro-5-(6-fluoro-1H-pyrazolo[4,3-b]pyridin-5-yl)-1-oxo-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

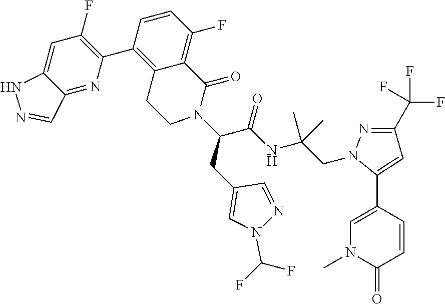 [SMILES: Cn1cc(-c2cc(C(F)(F)F)nn2CC(C)(C)NC(=O)[C@@H](Cc2cnn(C(F)F)c2)N2CCc3c(-c4nc5cn[nH]c5cc4F)ccc(F)c3C2=O)ccc1=O]

To a solution of (2R)-3-(1-(difluoromethyl)-1H-pyrazol-4-yl)-2-(8-fluoro-5-(6-fluoro-1-(tetrahydro-2H-pyran-2-yl)-1H-pyrazolo[4,3-b]pyridin-5-yl)-1-oxo-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (138 mg, 141 μmol) in MeOH (3 mL) was added hydrogen chloride (3.00 mL, 2 M in MeOH, 6.00 mmol). The reaction mixture was stirred at RT for 2 h. The mixture was concentrated under reduced pressure. The residue was purified by prep-TLC (DCM/MeOH 10:1, R_{f}=0.5) to obtain the title compound (112.1 mg, 94% yield, 93% purity) as a white solid. ES-MS m/z 785.2 (M+H).

The compounds in the following table were prepared in similar manner as described in Example 22 using the appropriate THP protected compound prepared previously and either TFA or HCl in a solvent such as DCM, MeOH or Water/Dioxane. Reaction conditions ranging from 1 to 12 h, 15 to 25° C. and 2 to 16 h. Reactants can be added in different orders or in differing equivalency, and purification methods were adjusted to suit the compounds. Such variances would be apparent to one skilled in the art.

TABLE 47

| | | | ES-MS |
|---|---|---|---|
| Example | Chemical Name | Structure | m/z |
| 23^{a} | (R)-3-(1- (Difluoromethyl)-1H- pyrazol-4-yl)-2-(8- fluoro-5-(4-fluoro-1H- pyrazolo[3,4-c]pyridin- 5-yl)-1-oxo-3,4- dihydroisoquinolin- 2(1H)-yl)-N-(2-methyl- 1-(5-(1-methyl-6-oxo- 1,6-dihydropyridin-3- yl)-3-(trifluoromethyl)- 1H-pyrazol-1-yl)propan- 2-yl)propanamide | 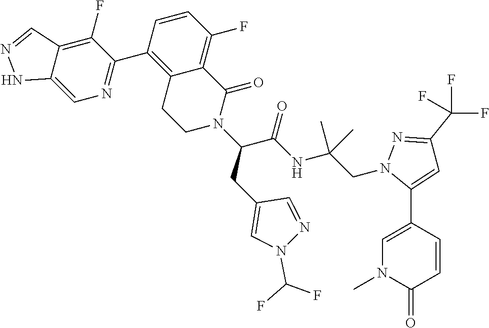 [SMILES: Cn1cc(-c2cc(C(F)(F)F)nn2CC(C)(C)NC(=O)[C@@H](Cc2cnn(C(F)F)c2)N2CCc3c(-c4ncc5[nH]ncc5c4F)ccc(F)c3C2=O)ccc1=O] | 785.2 (M + H) |
| 24^{b} | (R)-N-(1-(5-(1H-1,2,3- Triazol-5-yl)-3- (trifluoromethyl)-1H- pyrazol-1-yl)-2- methylpropan-2-yl)-2- (5-(5-cyano-1-methyl- 1H-indazol-6-yl)-8- fluoro-1-oxo-3,4- dihydroisoquinolin- 2(1H)-yl)-3-(1-methyl- 1H-pyrazol-4- yl)propanamide | 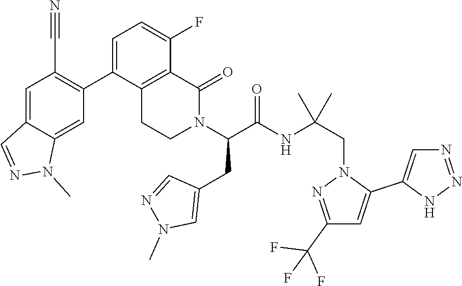 [SMILES: Cn1cc(C[C@H](C(=O)NC(C)(C)Cn2nc(C(F)(F)F)cc2-c2cnn[nH]2)N2CCc3c(-c4cc5c(cnn5C)cc4C#N)ccc(F)c3C2=O)cn1] | 729.3 (M + H) |
| 25^{c} | (R)-N-(1-(5-(2H-1,2,3- Triazol-4-yl)-3- (trifluoromethyl)-1H- pyrazol-1-yl)-2- methylpropan-2-yl)-2- (5-(6-cyano-1H- pyrazolo[4,3-b]pyridin- 5-yl)-8-fluoro-1-oxo- 3,4-dihydroisoquinolin- 2(1H)-yl)-3-(1-methyl- 1H-pyrazol-4- yl)propanamide | 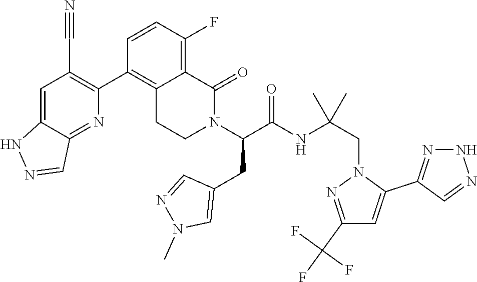 [SMILES: Cn1cc(C[C@H](C(=O)NC(C)(C)Cn2nc(C(F)(F)F)cc2-c2cn[nH]n2)N2CCc3c(-c4nc5cn[nH]c5cc4C#N)ccc(F)c3C2=O)cn1] | 716.3 (M + H) |
| 26^{d} | (R)-N-(1-(5-(1H-1,2,3- Triazol-5-yl)-3- (trifluoromethyl)-1H- pyrazol-1-yl)-2- methylpropan-2-yl)-2- (5-(6-cyano-1H-indazol- 5-yl)-8-fluoro-1-oxo- 3,4-dihydroisoquinolin- 2(1H)-yl)-3-(1-methyl- 1H-pyrazol-4- yl)propanamide | 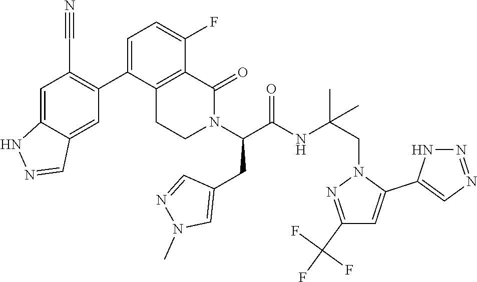 [SMILES: Cn1cc(C[C@H](C(=O)NC(C)(C)Cn2nc(C(F)(F)F)cc2-c2cnn[nH]2)N2CCc3c(-c4cc5cn[nH]c5cc4C#N)ccc(F)c3C2=O)cn1] | 715.2 (M + H) |
| 27^{e} | (R)-N-(1-(5-(1H-1,2,3- Triazol-5-yl)-3- (trifluoromethyl)-1H- pyrazol-1-yl)-2- methylpropan-2-yl)-3- (1-(difluoromethyl)-1H- pyrazol-4-yl)-2-(8- fluoro-5-(6-fluoro-1H- pyrazolo[3,4-b]pyridin- 5-yl)-1-oxo- 3,4-dihydroisoquinolin- 2(1H)-yl)propanamide | 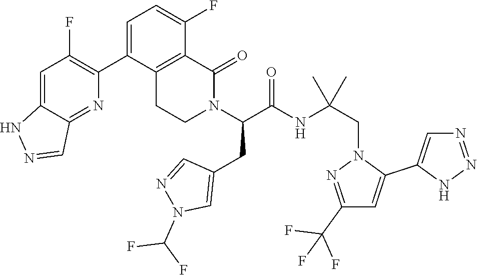 [SMILES: CC(C)(Cn1nc(C(F)(F)F)cc1-c1cnn[nH]1)NC(=O)[C@@H](Cc1cnn(C(F)F)c1)N1CCc2c(-c3nc4cn[nH]c4cc3F)ccc(F)c2C1=O] | 745.3 (M + H) |
| 28^{f} | (R)-2-(5-(6-Cyano-1H- indazol-5-yl)-8-fluoro-1- oxo-3,4-dihydroisoquinolin- 2(1H)-yl)-N-(1-(5-(5- methoxy-1,2,4- thiadiazol-3-yl)-3- (trifluoromethyl)-1H- pyrazol-1-yl)-2- methylpropan-2-yl)-3- (1-methyl-1H-pyrazol-4- yl)propanamide | 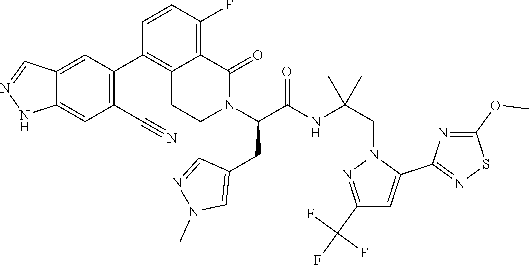 [SMILES: COc1nc(-c2cc(C(F)(F)F)nn2CC(C)(C)NC(=O)[C@@H](Cc2cnn(C)c2)N2CCc3c(-c4cc5cn[nH]c5cc4C#N)ccc(F)c3C2=O)ns1] | 762.4 (M + H) |
| 29^{g} | (R)-2-(5-(6-Cyano-1H- pyrazolo[4,3-b]pyridin- 5-yl)-8-fluoro-1-oxo- 3,4-dihydroisoquinolin- 2(1H)-yl)-N-(1-(5-(5- hydroxy-1,2,4- thiadiazol-3-yl)-3- (trifluoromethyl)-1H- pyrazol-1-yl)-2- methylpropan-2-yl)-3- (1-methyl-1H-pyrazol-4- yl)propanamide | 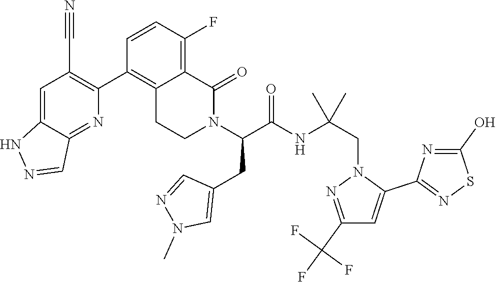 [SMILES: Cn1cc(C[C@H](C(=O)NC(C)(C)Cn2nc(C(F)(F)F)cc2-c2nsc(O)n2)N2CCc3c(-c4nc5cn[nH]c5cc4C#N)ccc(F)c3C2=O)cn1] | 749.8 (M + H) |
| 30^{h} | 2-Morpholinoethyl 3- ((R)-2-(5-(6-cyano-1H- indazol-5-yl)-8-fluoro-1- oxo-3,4-dihydroisoquinolin- 2(1H)-yl)-3-(1-methyl- 1H-pyrazol-4- yl)propanamido)-2-(4- fluorophenyl)-3- methylbutanoate | 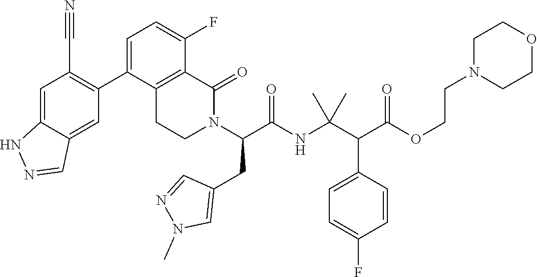 [SMILES: Cn1cc(C[C@H](C(=O)NC(C)(C)C(C(=O)OCCN2CCOCC2)c2ccc(F)cc2)N2CCc3c(-c4cc5cn[nH]c5cc4C#N)ccc(F)c3C2=O)cn1] | 765.3 (M + H) |
| 31ⁱ | 1-(Propionyloxy)ethyl 3- ((R)-2-(5-(6-cyano-1H- indazol-5-yl)-8-fluoro-1- oxo-3,4-dihydroisoquinolin- 2(1H)-yl)-3-(1-methyl- 1H-pyrazol-4- yl)propanamido)-2-(4- fluorophenyl)-3- methylbutanoate | 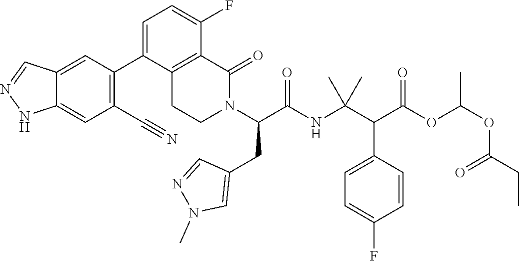 [SMILES: CCC(=O)OC(C)OC(=O)C(c1ccc(F)cc1)C(C)(C)NC(=O)[C@@H](Cc1cnn(C)c1)N1CCc2c(-c3cc4cn[nH]c4cc3C#N)ccc(F)c2C1=O] | 752.2 (M + H) |
| 32^{j} | Ethyl 3-((R)-2-(5-(6- cyano-1H-pyrazolo[4,3- b]pyridin-5-yl)-8-fluoro- 1-oxo-3,4-dihydroisoquinolin- 2(1H)-yl)-3-(1-methyl- 1H-pyrazol-4-yl)propanamido)-3- methyl-2-(4-(trifluoromethyl)-1H- pyrazol-1-yl)butanoate | 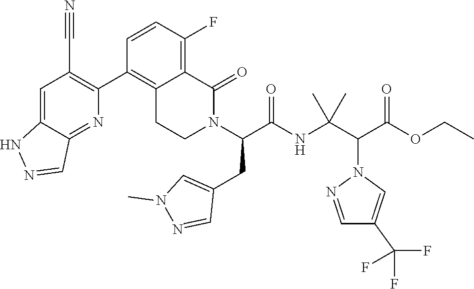 [SMILES: CCOC(=O)C(n1cc(C(F)(F)F)cn1)C(C)(C)NC(=O)[C@@H](Cc1cnn(C)c1)N1CCc2c(-c3nc4cn[nH]c4cc3C#N)ccc(F)c2C1=O] | 721.2 (M + H) |
| 33^{k} | (2R)-2-(5-(6-Cyano-1H- indazol-5-yl)-8-fluoro-1- oxo-3,4-dihydroisoquinolin- 2(1H)-yl)-N-(1-(4- fluorophenyl)-2-methyl- 1-(5-oxo-4,5-dihydro- 1,2,4-thiadiazol-3- yl)propan-2-yl)-3-(1- methyl-1H-pyrazol-4-yl) propanamide | 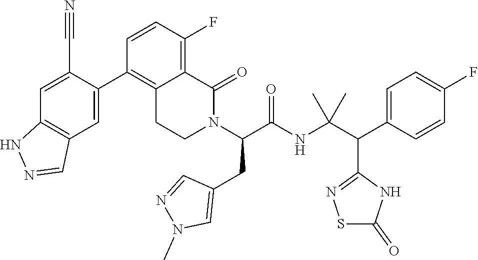 [SMILES: Cn1cc(C[C@H](C(=O)NC(C)(C)C(c2ccc(F)cc2)c2nsc(=O)[nH]2)N2CCc3c(-c4cc5cn[nH]c5cc4C#N)ccc(F)c3C2=O)cn1] | 708.1 (M + H) |
| 34^{l} | (R)-2-(5-(6-Cyano-1H- pyrazolo[4,3-b]pyridin- 5-yl)-8-fluoro-1-oxo- 3,4-dihydroisoquinolin- 2(1H)-yl)-N-(1-(5- cyclopropyl-1H-pyrazol- 1-yl)-2-methylpropan-2-yl)-3-(1- methyl-1H-pyrazol-4-yl) propanamide | 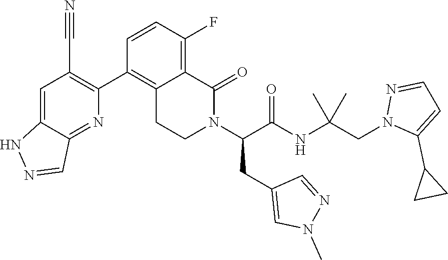 [SMILES: Cn1cc(C[C@H](C(=O)NC(C)(C)Cn2nccc2C2CC2)N2CCc3c(-c4nc5cn[nH]c5cc4C#N)ccc(F)c3C2=O)cn1] | 621.8 (M + H) |

^{a}From (2R)-3-(1-(difluoromethyl)-1H-pyrazol-4-yl)-2-(8-fluoro-5-(4-fluoro-1-(tetrahydro-2H-pyran-2-yl)-1H-pyrazolo[3,4-c]pyridin-5-yl)-1-oxo-3,4-
dihydroisoquinolin-2(1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide
^{b}From (2R)-2-(5-(5-cyano-1-methyl-1H-indazol-6-yl)-8-fluoro-1-oxo-3,4-dihydroisoquinolin-2(1H)-yl)-N-(2-methyl-1-(5-(2-(tetrahydro-2H-pyran-2-yl)-
2H-1,2,3-triazol-4-yl)-3-(trifluoromethyl)-1H-pyrazol-1-yl)propan-2-yl)-3-(1-methyl-1H-pyrazol-4-yl)propanamide
^{c}From (2R)-2-(5-(6-cyano-1-(tetrahydro-2H-pyran-2-yl)-1H-pyrazolo[4,3-b]pyridin-5-yl)-8-fluoro-1-oxo-3,4-dihydroisoquinolin-2(1H)-yl)-N-(2-methyl-
1-(5-(2-(tetrahydro-2H-pyran-2-yl)-2H-1,2,3-triazol-4-yl)-3-(trifluoromethyl)-1H-pyrazol-1-yl)propan-2-yl)-3-(1-methyl-1H-pyrazol-4-yl)propanamide
^{d}From (2R)-N-(1-(5-(2H-1,2,3-triazol-4-yl)-3-(trifluoromethyl)-1H-pyrazol-1-yl)-2-methylpropan-2-yl)-2-(5-(6-cyano-1-(tetrahydro-2H-pyran-2-yl)-1H-
indazol-5-yl)-8-fluoro-1-oxo-3,4-dihydroisoquinolin-2(1H)-yl)-3-(1-methyl-1H-pyrazol-4-yl)propanamide
^{e}From (2R)-N-(1-(5-(1H-1,2,3-triazol-5-yl)-3-(trifluoromethyl)-1H-pyrazol-1-yl)-2-methylpropan-2-yl)-3-(1-(difluoromethyl)-1H-pyrazol-4-yl)-2-(8-
fluoro-5-(6-fluoro-1-(tetrahydro-2H-pyran-2-yl)-1H-pyrazolo[4,3-b]pyridin-5-yl)-1-oxo-3,4-dihydroisoquinolin-2(1H)-yl)propanamide
^{f}From (2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-N-(1-(5-(5-methoxy-1,2,4,-
thiadiazol-3-yl)-3-(trifluoromethyl)-1H-pyrazol-1-yl)-2-methylpropan-2-yl)-3-(1-methyl-1H-pyrazol-4-yl)propanamide
^{g}From (2R)-2-(5-(6-cyano-1-(tetrahydro-2H-pyran-2-yl)-1H-pyrazolo[4,3-b]pyridin-5-yl)-8-fluoro-1-oxo-3,4-dihydroisoquinolin-2(1H)-yl)-N-(1-(5-(5-
hydroxy-1,2,4-thiadiazol-3-yl)-3-(trifluoromethyl)-1H-pyrazol-1-yl)-2-methylpropan-2-yl)-3-(1-methyl-1H-pyrazol-4-yl)propanamide
^{h}From 2-morpholinoethyl 3-((2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-3-(1-
methyl-1H-pyrazol-4-yl)propanamide
ⁱFrom 1-(propionyloxy)ethyl 3-((R)-2-(5-(6-cyano-1H-indazol-5-yl)-8-fluoro-1-oxo-3,4-dihydroisoquinolin-2(1H)-yl)-3-(1-methyl-1H-pyrazol-4-yl)
propanamido)-2-(4-fluorophenyl)-3-methylbutanoate
^{j}From ethyl 3-((2R)-2-(5-(6-cyano-1-(tetrahydro-2H-pyran-2-yl)-1H-pyrazolo[4,3-b]pyridin-5-yl)-8-fluoro-1-oxo-3,4-dihydroisoquinolin-2(1H)-yl)-3-
(1-methyl-1H-pyrazol-4-yl)propanamido)-3-methyl-2-(4-(trifluoromethyl)-1H-pyrazol-1-yl)butanoate
^{k}From (2R)-2-(5-(6-cyano-1-(tetrahydro-2H-pyran-2-yl)-1H-indazol-5-yl)-8-fluoro-1-oxo-3,4-dihydroisoquinolin-2(1H)-yl)-N-(1-(4-fluorophenyl)-2-
methyl-1-(5-oxo-4,5-dihydro-1,2,4-thiadiazol-3-yl)propan-2-yl)-3-(1-methyl-1H-pyrazol-4-yl)propanamide
^{l}From (2R)-2-(5-(6-cyano-1-(tetrahydro-2H-pyran-2-yl)-1H-pyrazolo[4,3-b]pyridin-5-yl)-8-fluoro-1-oxo-3,4-dihydroisoquinolin-2(1H)-yl)-N-(1-(5-
cyclopropyl-1H-pyrazol-1-yl)-2-methylpropan-2-yl)-3-(1-methyl-1H-pyrazol-4-yl)propanamide

### Example 35

### 3-((R)-2-(5-(6-Cyano-1H-pyrazolo[4,3-b]pyridin-5-yl)-8-fluoro-1-oxo-3,4-dihydroisoquinolin-2(1H)-yl)-3-(1-methyl-1H-pyrazol-4-yl)propanamido)-3-methyl-2-(4-(trifluoromethyl)-1H-pyrazol-1-yl)butanoic acid

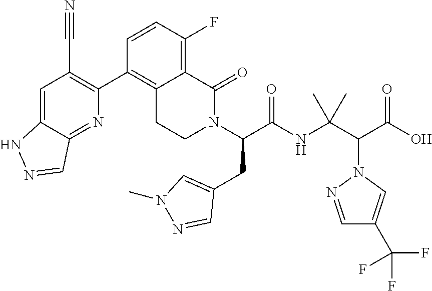 [SMILES: Cn1cc(C[C@H](C(=O)NC(C)(C)C(C(=O)O)n2cc(C(F)(F)F)cn2)N2CCc3c(-c4nc5cn[nH]c5cc4C#N)ccc(F)c3C2=O)cn1]

To a solution of ethyl 3-((R)-2-(5-(6-cyano-1H-pyrazolo[4,3-b]pyridin-5-yl)-8-fluoro-1-oxo-3,4-dihydroisoquinolin-2(1H)-yl)-3-(1-methyl-1H-pyrazol-4-yl)propanamido)-3-methyl-2-(4-(trifluoromethyl)-1H-pyrazol-1-yl)butanoate (210 mg, 60% purity, 175 μmol) in THF (2 mL) and EtOH (2 mL) was added lithium hydroxide (874 μL, 1 M in water, 874 μmol). The mixture was stirred at 25° C. for 2 h. The mixture was adjusted to pH 3 with 1 M aqueous HCl, then the mixture was extracted with EtOAc (20 mL×2). The combined organic layers were washed with sat. aq. NaCl (20 mL×2), dried over sodium sulfate, filtered, and the filtrate concentrated under reduced pressure. The residue was purified by prep-HPLC [column: F-Prepulite XP tC 18, 40 mm×200 mm, 7 μm; mobile phase: solvent A-water (NH₄HCO₃), solvent B-ACN, gradient-2%-42% solvent B] to obtain the title compound (65.2 mg, 53%) as a white solid. ES-MS m/z 693.2 (M+H).

### Example 36

### (R)-2-(5-(6-Cyano-1-methyl-1H-indazol-5-yl)-8-fluoro-1-oxo-3,4-dihydroisoquinolin-2 (1H)-yl)-N-(1-(5-(5-hydroxy-1,2,4-thiadiazol-3-yl)-3-(trifluoromethyl)-1H-pyrazol-1-yl)-2-methylpropan-2-yl)-3-(1-methyl-1H-pyrazol-4-yl)propanamide

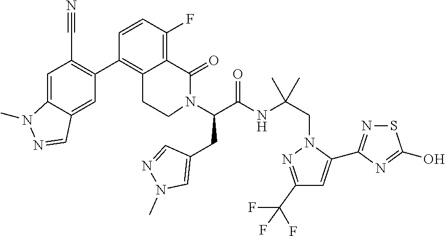 [SMILES: Cn1cc(C[C@H](C(=O)NC(C)(C)Cn2nc(C(F)(F)F)cc2-c2nsc(O)n2)N2CCc3c(-c4cc5cnn(C)c5cc4C#N)ccc(F)c3C2=O)cn1]

To a solution of (R)-2-(5-(6-cyano-1-methyl-1H-indazol-5-yl)-8-fluoro-1-oxo-3,4-dihydroisoquinolin-2(1H)-yl)-3-(1-methyl-1H-pyrazol-4-yl)propanoic acid (45 mg, 88% purity, 84 μmol), diethyl (4-oxobenzo[d][1,2,3]triazin-3 (4H)-yl)phosphate (50 mg, 0.17 mmol), and 3-(1-(2-amino-2-methylpropyl)-3-(trifluoromethyl)-1H-pyrazol-5-yl)-1,2,4-thiadiazol-5-ol hydrochloride (39 mg, 86% purity, 98 μmol) in DMA (1 mL) was added 4-methylmorpholine (92 μL, 0.84 mmol) at 25° C. The mixture was stirred at 25° C. for 16 h. The reaction mixture was diluted with water (15 mL) and extracted with EtOAc (15 mL×2). The combined organic layers were washed with sat. aq. NaCl (20 mL×2), dried over sodium sulfate, filtered, and the filtrate concentrated under reduced pressure. The residue was purified by prep-TLC (DCM/MeOH 20:1, R_{f}=0.3), and then further purified by SFC [column: Daicel CHIRALPAK® AS, 250 mm×30 mm, 10 μm; mobile phase: 75% CO₂-25% (EtOH+0.1% NH₄OH)] to obtain the title compound (11 mg, 17%) as a white solid. ES-MS m/z 762.2 (M+H).

The compounds in the following table were prepared in similar manner as described in Example 36 using the appropriate carboxylic acid and primary amine previously described. Reactants can be added in different orders or in differing equivalency, and purification methods were adjusted to suit the compounds. Such variances would be apparent to one skilled in the art.

TABLE 48

| | | | ES-MS |
|---|---|---|---|
| Example | Chemical Name | Structure | m/z |
| 37 | (R)-2-(5-(6-Cyano-1-methyl-1H- indazol-5-yl)-8-fluoro-1-oxo- 3,4-dihydroisoquinolin-2(1H)-yl)-N- (1-(5-(5-hydroxy-1,3,4- thiadiazol-2-yl)-3-(trifluoro- methyl)-1H-pyrazol-1-yl)-2- methylpropan-2-yl)-3-(1-methyl- 1H-pyrazol-4-yl)propanamide | 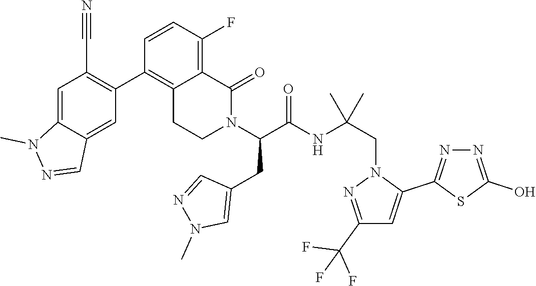 [SMILES: Cn1cc(C[C@H](C(=O)NC(C)(C)Cn2nc(C(F)(F)F)cc2-c2nnc(O)s2)N2CCc3c(-c4cc5cnn(C)c5cc4C#N)ccc(F)c3C2=O)cn1] | 762.1 (M + H) |

### Example 38

### (R)-3-(1-(Difluoromethyl)-1H-pyrazol-4-yl)-2-(8-fluoro-5-(4-fluoro-2-methyl-2H-pyrazolo[3,4-c]pyridin-5-yl)-1-oxo-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

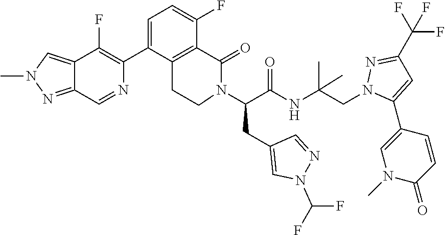 [SMILES: Cn1cc2c(F)c(-c3ccc(F)c4c3CCN([C@H](Cc3cnn(C(F)F)c3)C(=O)NC(C)(C)Cn3nc(C(F)(F)F)cc3-c3ccc(=O)n(C)c3)C4=O)ncc2n1]

A mixture of (R)-3-(1-(difluoromethyl)-1H-pyrazol-4-yl)-2-(8-fluoro-5-(4-fluoro-1H-pyrazolo[3,4-c]pyridin-5-yl)-1-oxo-3,4-dihydroisoquinolin-2 (1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (90.95 mg, 0.12 mmol) and cesium carbonate (62.8 mg, 0.19 mmol) was purged with N₂. DMF (1.16 mL) and methyl iodide (10 mL, 0.15 mmol) were added, and the resulting mixture was stirred at RT for 1 h. The reaction was diluted with EtOAc and poured into water. The layers were separated, and the organic layer was extracted with EtOAc (2×20 mL). The organic layers were combined, washed with sat. aq. NaCl, dried over Na₂SO₄, filtered and concentrated under reduced pressure. The residue was purified by reverse phase prep-HPLC [column: Phenomenex Kinetex EVO C18, 100×30 mm, 5 mm; mobile phase: gradient of 33%-67% ACN in water (with 0.1% FA)] to give the title compound (first-eluting isomer, 29.7 mg, 32%) as a white solid. ES-MS m/z 799.2 (M+H)

### Example 39

### (R)-2-(5-(2-Cyano-4-(2-methyl-2H-pyrazolo[3,4-b]pyridin-5-yl)phenyl)-8-fluoro-1-oxo-3,4-dihydroisoquinolin-2 (1H)-yl)-N-(1-(5-(5-hydroxy-1,2,4-thiadiazol-3-yl)-3-(trifluoromethyl)-1H-pyrazol-1-yl)-2-methylpropan-2-yl)-3-(1-methyl-1H-pyrazol-4-yl)propanamide

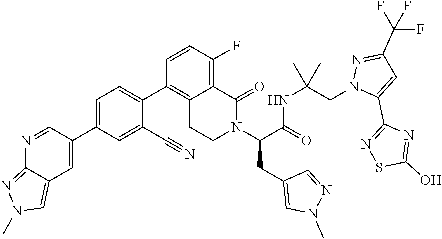 [SMILES: Cn1cc(C[C@H](C(=O)NC(C)(C)Cn2nc(C(F)(F)F)cc2-c2nsc(O)n2)N2CCc3c(-c4ccc(-c5cnc6nn(C)cc6c5)cc4C#N)ccc(F)c3C2=O)cn1]

To a solution of (R)-2-(5-(2-cyano-4-(2-methyl-2H-pyrazolo[3,4-b]pyridin-5-yl)phenyl)-8-fluoro-1-oxo-3,4-dihydroisoquinolin-2 (1H)-yl)-N-(1-(5-(5-methoxy-1,2,4-thiadiazol-3-yl)-3-(trifluoromethyl)-1H-pyrazol-1-yl)-2-methylpropan-2-yl)-3-(1-methyl-1H-pyrazol-4-yl)propanamide (210 mg, 88% purity, 217 μmol) in DMA (12 mL) was added pyridine hydrochloride (253 mg, 2.17 mmol) at 15° C. The mixture was stirred at 80° C. for 2 h, then concentrated. The residue was purified by prep-HPLC [column: F-Prepulite XP tC 18 40×200 mm, 7 μm; flow rate: 60 mL/min; mobile phase: solvent A=water+0.1% FA, solvent B=ACN, gradient 20-60% B then 100% B) to provide the title compound (74.7 mg, 41% yield, 99% purity) as a red solid. ES-MS m/z 839.3 (M+H).

The compounds in the following table were prepared in similar manner as described in Example 39 using the appropriate methoxy thiadiazole compound. Reactants can be added in different orders or in differing equivalency, and purification methods were adjusted to suit the compounds. The reaction solvent can be either DMA or DMF. The reaction temperature can be between 8° and 100° C. Such variances would be apparent to one skilled in the art.

TABLE 49

| | | | ES-MS |
|---|---|---|---|
| Example | Chemical Name | Structure | m/z |
| 40^{a} | (R)-2-(5-(6-Cyano-1H-indazol- 5-yl)-8-fluoro-1-oxo- 3,4-dihydroisoquinolin-2(1H)- yl)-N-(1-(5-(5-hydroxy-1,2,4- thiadiazol-3-yl)-3- (trifluoromethyl)-1H-pyrazol- 1-yl)-2-methylpropan-2-yl)-3- (1-methyl-1H-pyrazol-4- yl)propanamide | 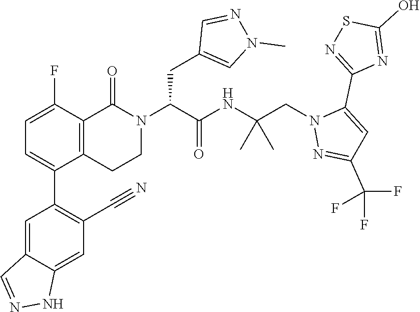 [SMILES: Cn1cc(C[C@H](C(=O)NC(C)(C)Cn2nc(C(F)(F)F)cc2-c2nsc(O)n2)N2CCc3c(-c4cc5cn[nH]c5cc4C#N)ccc(F)c3C2=O)cn1] | 748.7 (M + H) |

^{a}Solvent used: DMF; heated at 100° C. for 3 hours.

### Example 41

### 3-((R)-2-(5-(6-Cyano-1H-indazol-5-yl)-8-fluoro-1-oxo-3,4-dihydroisoquinolin-2(1H)-yl)-3-(1-methyl-1H-pyrazol-4-yl)propanamido)-3-methyl-2-(3-(trifluoromethyl)phenyl)butanoic acid (Isomer 2)

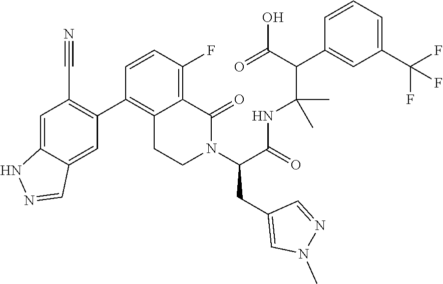 [SMILES: Cn1cc(C[C@H](C(=O)NC(C)(C)C(C(=O)O)c2cccc(C(F)(F)F)c2)N2CCc3c(-c4cc5cn[nH]c5cc4C#N)ccc(F)c3C2=O)cn1]

The title compound was isolated from a 1:1 mixture of diastereomers (Preparation 177, 100 mg, dissolved in 6 mL MeOH) by preparative SFC [column: R,R Whelk-O1, 21×150 mm, 5 μm; mobile phase: isocratic 30% EtOH (0.5% DMEA): 70% CO₂; flow rate: 80 mL/min; column temperature=40° C.]. The second-eluting product was concentrated to provide the title compound (39 mg, 39% recovery) as a white solid. ES-MS m/z 702.4 (M+H).

The compounds in the following table were isolated as described in Example 41 using the appropriate diastereomer mixtures described previously. In each case the chosen diastereomer was the second eluting product. Mobile phases may contain between 30-35% EtOH (0.5% DMEA): 65-70% CO₂.

TABLE 50

| | | | ES-MS |
|---|---|---|---|
| Example | Chemical Name | Structure | m/z |
| 42 | 3-((R)-2-(5- (6-Cyano-1H- pyrazolo[4,3-b]pyridin- 5-yl)-8-fluoro-1-oxo- 3,4-dihydroisoquinolin- 2(1H)-yl)-3- (1-methyl-1H-pyrazol-4- yl)propanamido)-3-methyl-2- (3-(trifluoromethyl)phenyl) butanoic acid (Isomer 2) | 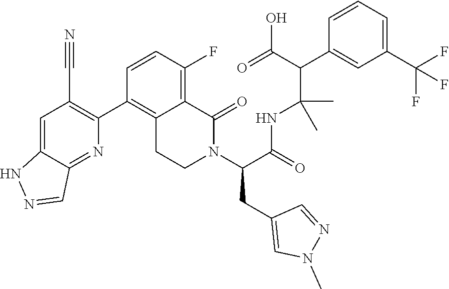 [SMILES: Cn1cc(C[C@H](C(=O)NC(C)(C)C(C(=O)O)c2cccc(C(F)(F)F)c2)N2CCc3c(-c4nc5cn[nH]c5cc4C#N)ccc(F)c3C2=O)cn1] | 703.4 (M + H) |
| 43 | 3-((R)-2-(5-(6-Cyano- 1H-indazol- 5-yl)-8-fluoro-1-oxo- 3,4-dihydroisoquinolin- 2(1H)- yl)-3-(1-methyl-1H- pyrazol-4- y)propanamido)-2-(4- fluorophenyl)-3- methylbutanoic acid (Isomer 2) | 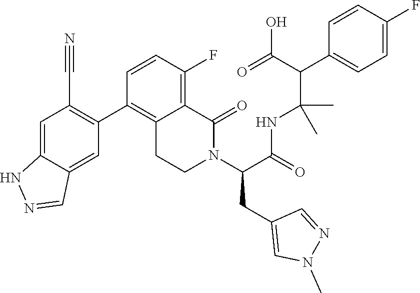 [SMILES: Cn1cc(C[C@H](C(=O)NC(C)(C)C(C(=O)O)c2ccc(F)cc2)N2CCc3c(-c4cc5cn[nH]c5cc4C#N)ccc(F)c3C2=O)cn1] | 652.4 (M + H) |
| 44 | 3-((R)-2-(5- (6-Cyano-1H- pyrazolo[4,3-b]pyridin- 5-yl)-8-fluoro-1-oxo- 3,4-dihydroisoquinolin- 2(1H)-yl)- 3-(1-methyl-1H-pyrazol-4- yl)propanamido)-2-(4- fluorophenyl)-3- methylbutanoic acid (Isomer 2) | 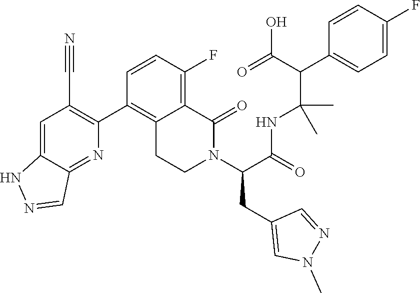 [SMILES: Cn1cc(C[C@H](C(=O)NC(C)(C)C(C(=O)O)c2ccc(F)cc2)N2CCc3c(-c4nc5cn[nH]c5cc4C#N)ccc(F)c3C2=O)cn1] | 653.4 (M + H) |

### Example 45

### (R)-2-(5-(3,6-Difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

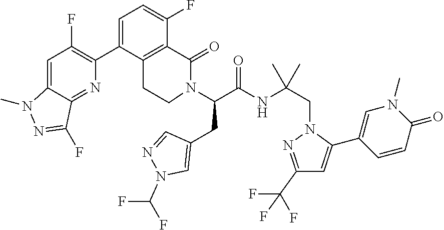 [SMILES: Cn1cc(-c2cc(C(F)(F)F)nn2CC(C)(C)NC(=O)[C@@H](Cc2cnn(C(F)F)c2)N2CCc3c(-c4nc5c(F)nn(C)c5cc4F)ccc(F)c3C2=O)ccc1=O]

To a mixture of (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (400 mg, 692 μmol, 90% purity), 5-(1-(2-amino-2-methylpropyl)-3-(trifluoromethyl)-1H-pyrazol-5-yl)-1-methylpyridin-2(1H)-one hydrochloride (324 mg, 830 μmol, 90% purity), and DEPBT (414 mg, 1.38 mmol) in DMA (20 mL) was added N-methylmorpholine (0.76 mL, 6.92 mmol). The mixture was stirred at 25° C. for 16 h. Then, the mixture was diluted with water (50 mL) and extracted with EtOAc (40 mL×3). The combined organic layers were washed with sat. aq. NaCl (30 mL×2), dried over Na₂SO₄, filtered, and the filtrate concentrated under reduced pressure. The residue was purified on silica gel, eluting with 0-4% MeOH in DCM to afford 360 mg of a yellow solid. This was re-purified by chiral SFC [column: DAICEL CHIRALPAK AD (250 mm×30 mm, 10 μm), mobile phase: 30% IPA with 0.1% NH₄OH in supercritical CO₂]. The first eluting enantiomer was concentrated under reduced pressure and lyophilized to obtain the title compound (216 mg, 98.4% purity, 61.5% yield) as a white solid. ES-MS m/z 817 (M+H).

### Example 46

### (R)-2-(5-(6-Cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

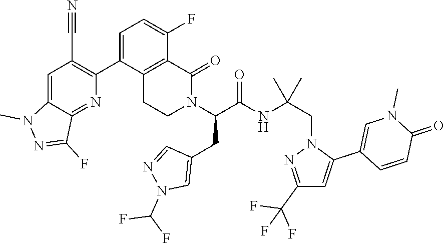 [SMILES: Cn1cc(-c2cc(C(F)(F)F)nn2CC(C)(C)NC(=O)[C@@H](Cc2cnn(C(F)F)c2)N2CCc3c(-c4nc5c(F)nn(C)c5cc4C#N)ccc(F)c3C2=O)ccc1=O]

A mixture of 5-chloro-3-fluoro-1-methyl-1H-pyrazolo[4,3-b]pyridine-6-carbonitrile (38 mg, 0.13 mmol, 70% purity), (R)-(2-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-8-fluoro-1-oxo-1,2,3,4-tetrahydroisoquinolin-5-yl)boronic acid (0.11 g, 0.15 mmol, 95% purity), sodium carbonate (27 mg, 0.25 mmol), and XPhos Pd G3 (21 mg, 0.25 μmol) in 1,4-dioxane (1 mL) and water (0.1 mL) was degassed under vacuum and backfilled with nitrogen three times at 27° C. The mixture was then stirred at 90° C. for 1 h under an atmosphere of nitrogen. Then, the mixture was diluted with EtOAc (10 mL), filtered, and the filtrate was concentrated under reduced pressure, and purified by prep-TLC using 20:1 DCM-MeOH as the mobile phase to afford 99 mg of a sticky yellow solid. This product was re-purified by reverse phase HPLC (YMC Triart C18 150×25 mM, 5 μM) using 40-70% ACN in aq. 10 mM ammonium formate as the mobile phase. Fractions were concentrated under reduced pressure and lyophilized to obtain the title compound (46 mg, 95.4% purity, 42% yield) as a white solid. ES-MS m/z 824 (M+H).

### Example 47

### (R)-2-(5-(3-Chloro-6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide

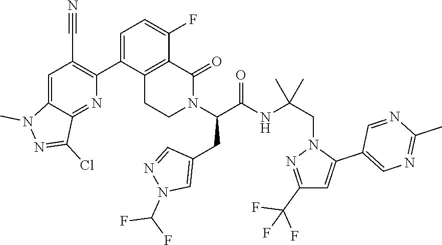

To a mixture of (R)-2-(5-bromo-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide (2.86 g, 4.01 mmol), bis(neopentyl glycolato)diboron (1.54 g, 6.82 mmol), potassium acetate (1.18 g, 12.0 mmol), and dichloro{bis[2-(diphenylphosphino)phenyl]ether}palladium(II) (287 mg, 401 μmol) was added 1,4-dioxane (20.0 mL). The mixture was placed under vacuum and backfilled with nitrogen gas 3 times then stirred at 80° C. for 1.5 h. The mixture was cooled to RT then 3,5-dichloro-1-methyl-1H-pyrazolo[4,3-b]pyridine-6-carbonitrile (820 mg, 3.61 mmol), K₃PO₄ (1.45 g, 6.83 mmol), XPhos Pd G4 (345 mg, 400 μmol) and water (11.6 mL, 644 mmol) were added. The mixture was placed under vacuum and backfilled with nitrogen gas 3 times and stirred at 80° C. for 2 h, then concentrated and the residue was purified on silica gel (330 g), eluting with a gradient of 20% to 30% of a premixed solution of (20% IPA: 80% acetone), in cyclohexane to provide the title compound (2.05 g, 62% yield) as a white solid. ES-MS m/z 825 (M+H).

### Example 48

### (R)-2-(5-(6-Cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide

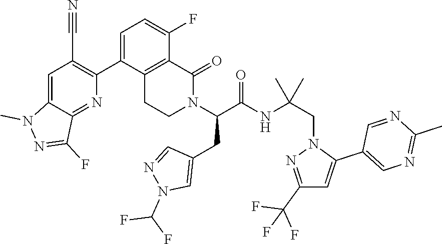

To a mixture of (R)-2-(5-bromo-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide (15.00 g, 21.02 mmol), bis(neopentyl glycolato)diboron (8.07 g, 35.7 mmol) and potassium acetate (6.19 g, 63.1 mmol) was added 1,4-dioxane (140.2 mL). Nitrogen gas was bubbled through the mixture for 10 min, then dichloro{bis[2-(diphenylphosphino)phenyl]ether}palladium(II) (1.50 g, 2.10 mmol) was added. The mixture was placed under vacuum and back filled with nitrogen gas three times, then stirred at 80° C. for 1 h. The mixture cooled to RT then 5-chloro-3-fluoro-1-methyl-1H-pyrazolo[4,3-b]pyridine-6-carbonitrile (4.44 g, 21.1 mmol), K₃PO₄ (7.61 g, 35.9 mmol), and XPhos Pd G4 (1.82 g, 2.11 mmol) and water (60 mL) were added. The resulting mixture was vacuum degassed with nitrogen gas three times, stirred at 80° C. for 3 h, diluted with EtOAc (800 mL), washed with sat. aq. NaCl (1 L), dried over Na₂SO₄, filtered and concentrated. The residue was purified on silica gel, eluting with a gradient of 10% to 40% of a premixed solution of (20% IPA: 80% acetone) in cyclohexane to provide the title compound (15.50 g, 91% yield) as a white solid. ES-MS m/z 809 (M+H).

The compounds in the following table were prepared as described in Example 47 using the appropriate aryl halide. Reactions were run at 70-90° C. for 1-6 h, and purification methods were adjusted to suit the compounds. Such variances would be apparent to one skilled in the art.

TABLE 51

| | | | ES-MS |
|---|---|---|---|
| Example | Chemical Name | Structure | m/z |
| 49 | (R)-3-(1- (Difluoromethyl)- 1H-pyrazol-4-yl)-2- (8-fluoro-5-(5- fluoro-1-methyl-1H- pyrazolo[3,4- b]pyridin-6-yl)-1- oxo-3,4- dihydroisoquinolin- 2(1H)-yl)-N-(2- methyl-1-(5-(2- methylpyrimidin-5- yl)-3- (trifluoromethyl)- 1H-pyrazol-1- yl)propan-2- yl)propanamide | 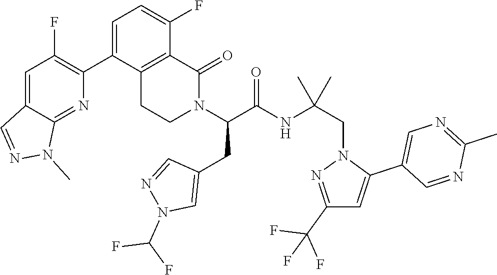 [SMILES: Cc1ncc(-c2cc(C(F)(F)F)nn2CC(C)(C)NC(=O)[C@@H](Cc2cnn(C(F)F)c2)N2CCc3c(-c4nc5c(cnn5C)cc4F)ccc(F)c3C2=O)cn1] | 784 (M + H) |
| 50 | (R)-2-(5-(5-Cyano- 1-methyl-1H- pyrazolo[3,4- b]pyridin-6-yl)-8- fluoro-1-oxo-3,4- dihydroisoquinolin- 2(1H)-yl)-3-(1- (difluoromethyl)-1H- pyrazol-4-yl)-N-(2- methyl-1-(5-(2- methylpyrimidin-5- yl)-3- (trifluoromethyl)- 1H-pyrazol-1- yl)propan-2- yl)propanamide | 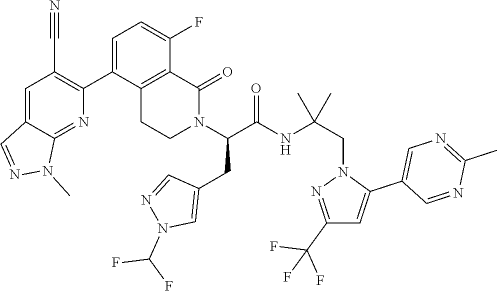 [SMILES: Cc1ncc(-c2cc(C(F)(F)F)nn2CC(C)(C)NC(=O)[C@@H](Cc2cnn(C(F)F)c2)N2CCc3c(-c4nc5c(cnn5C)cc4C#N)ccc(F)c3C2=O)cn1] | 791 (M + H) |
| 51 | (R)-2-(5-(6-cyano-1- methyl-1H- pyrazolo[4,3- b]pyridin-5-yl)-8- fluoro-1-oxo-3,4- dihydroisoquinolin- 2(1H)-yl)-3-(1- (difluoromethyl)-1H- pyrazol-4-yl)-N-(2- methyl-1-(5-(2- methylpyrimidin-5- yl)-3- (trifluoromethyl)- 1H-pyrazol-1- yl)propan-2- yl)propanamide | 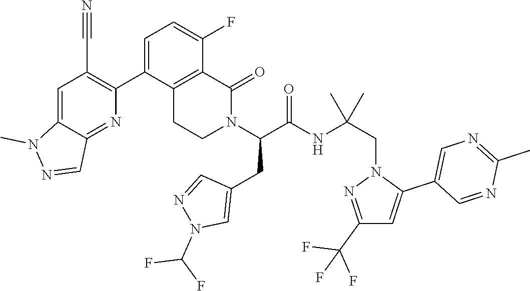 | 791 (M + H) |
| 52 | (R)-2-(5-(3,6- difluoro-1-methyl- 1H-pyrazolo[4,3- b]pyridin-5-yl)-8- fluoro-1-oxo-3,4- dihydroisoquinolin- 2(1H)-yl)-3-(1- (difluoromethyl)-1H- pyrazol-4-yl)-N-(2- methyl-1-(5-(2- methylpyrimidin-5- yl)-3- (trifluoromethyl)- 1H-pyrazol-1- yl)propan-2- yl)propanamide | 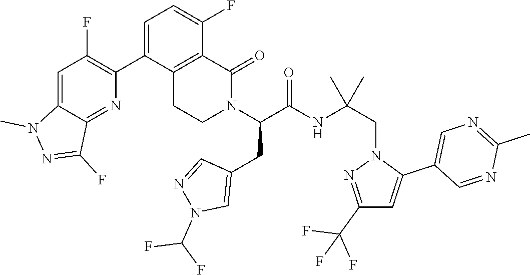 | 802 (M + H) |

### Example 53

### (R)-2-(5-(3-Chloro-6-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide

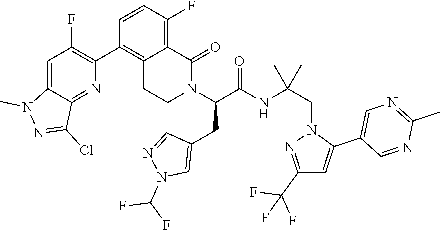

A mixture of 5-bromo-3-chloro-6-fluoro-1-methyl-1H-pyrazolo[4,3-b]pyridine (25 mg, 93 μmol, 98% purity), (R)-3-(1-(difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)propanamide (77 mg, 96 μmol, 95% purity), Na₂CO₃ (33 mg, 0.31 mmol), and Pd(dppf)Cl₂ (15 mg, 20 μmol) in 1,4-dioxane (2 mL) and water (0.2 mL) was degassed under reduced pressure and backfilled with nitrogen three times. The mixture was then stirred at 80° C. for 3 h under nitrogen. Then, the mixture was diluted with water (10 mL) and extracted with EtOAc (10 mL×2). The combined organic layers were washed with sat. aq. NaCl (15 mL×3), dried over Na₂SO₄, filtered, and the filtrate concentrated under reduced pressure. The residue was purified on silica gel, eluting with 0-6% MeOH in DCM. The product was then re-purified by prep-TLC using 10:1 DCM-MeOH as the mobile phase and the resulting material was lyophilized from 2:1 H₂O-ACN (15 mL) to obtain the title compound (45.0 mg, 97.7% purity, 58% yield) as a white solid. ES-MS m/z 818 (M+H).

### Example 54

### (R)-3-(1-(Difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)propanamide

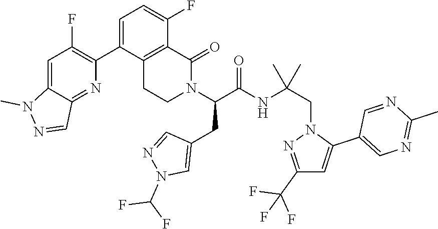

To a mixture of 5-chloro-6-fluoro-1-methyl-1H-pyrazolo[4,3-b]pyridine (49 mg, 0.26 mmol), Pd(dppf)Cl₂ (complex with DCM, 24 mg, 29 μmol) and cesium carbonate (175 mg, 537 μmol) under an atmosphere of nitrogen was added a nitrogen-sparged mixture of 1,4-dioxane (2.5 mL) and water (0.25 mL). The resulting mixture was degassed with bubbling nitrogen for 5 min, then (R)-3-(1-(difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)propanamide (202 mg, 266 μmol) was added and the mixture was heated to 70° C. overnight. The reaction was cooled to RT, filtered over a pad of diatomaceous earth, and rinsed with DCM. The resulting eluent was concentrated under nitrogen stream. The residue was purified by silica gel chromatography using a gradient of 10 to 60% (20% IPA in acetone) in cyclohexane, then re-purified by silica gel chromatography using a gradient of 10 to 25% (20% IPA in acetone) in cyclohexane. The product was then re-purified by reverse phase prep-HPLC [column: Evo 30×110 mm C-18; mobile phase: 23 to 58% ACN in aq. NH4HCO3] and dried under reduced pressure to give the title compound (50 mg, 24% yield) as an off-white solid. ES-MS m/z 784 (M+H).

### Example 55

### (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-methoxy-3-(trifluoromethyl)-1H-pyrazol-1-yl)-2-methylpropan-2-yl)propanamide

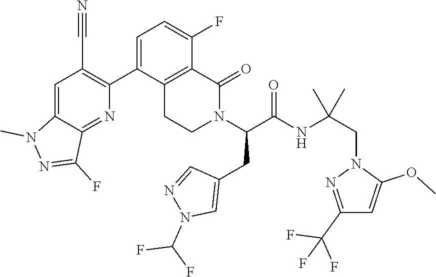

To a solution of 1-(5-methoxy-3-(trifluoromethyl)-1H-pyrazol-1-yl)-2-methylpropan-2-amine hydrochloride (86 mg, 60% wt, 0.95 Eq, 0.19 mmol), (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (110 mg, 95% wt, 1 eq, 198 μmol) and diethyl (4-oxo-1,2,3-benzotriazin-3-yl)phosphate (119 mg, 2.01 eq, 398 μmol) in DMA (5 mL) was added 4-methylmorpholine (0.28 g, 0.30 mL, 14 eq, 2.7 mmol). The reaction was stirred at RT for 16 h. The reaction was quenched by the addition of H₂O (20 mL) and extracted with EtOAc (20 mL×2). The combined organic layer was washed with saturated aqueous NaCl (20 mL×2), dried over anhydrous Na₂SO₄, filtered and concentrated under reduced pressure. The residue was purified by prep-HPLC (column: YMC Triart C18 250*30 mm*5 μm; mobile phase: [A: H₂O (0.225% FA); B: ACN]; B %: 40.00%-70.00%, 10.00 min; flow rate: 25.00 ml/min) to afford the title compound (106 mg, 98.5% purity, 71% yield, 69% e.e.) as a white solid. The material underwent chiral separation by SFC (column: REGIS (s,s) WHELK-01 (250 mm*30 mm, 10 μm); mobile phase: [A: CO₂; B: EtOH (0.1% NH₄OH)]; B %: 50.00%-50.00%, 3.00 min; flow rate: 80.00 g/min). The first eluting peak gave the title compound (65 mg, 98% purity, 61% yield, 99.3% e.e.) as a white solid. ES-MS m/z 747.1 (M+H).

### Example 56

### (2R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(1-methyl-2-oxopyrrolidin-3-yl)-3-(trifluoromethyl)-1H-pyrazol-1-yl)propan-2-yl)propanamide (isomer 1)

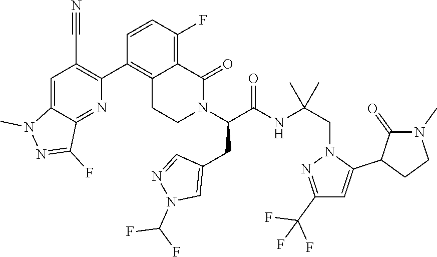

Example 56 was prepared in a manner substantially similar to that described in Example 55 to provide a diastereomer mixture from the appropriate carboxylic acid and primary amine. The product underwent chiral separation by SFC (column: DAICEL CHIRALPAK IG (250 mm*30 mm, 10 μm); mobile phase: [A: CO₂; B: IPA (0.1% NH₄OH)]; B %: 55.00%-55.00%, 5.00 min; flow rate: 80.00 g/min). The first eluting peak afforded the title compound (43.5 mg, 97% purity, 99.4% e.e., 31% yield) as a white solid. ES-MS m/z 814.2 (M+H)

The compounds in the following table were prepared in similar manner as described in Example 55 using (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid and the appropriate primary amine. Different reaction temperatures, reaction times and further purification by supercritical fluid chromatography (SFC) can be used to increase purity as desired. Such variances would be apparent to one skilled in the art.

TABLE 52

| | | | ES-MS |
|---|---|---|---|
| Example | Chemical Name | Structure | m/z |
| 57 | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-N-(1-(5-(1- cyclopropyl-2-oxo-1,2- dihydropyridin-4-yl)-3- (trifluoromethyl)-1H- pyrazol-1-yl)-2- methylpropan-2-yl)-3- (1-(difluoromethyl)-1H- pyrazol-4- yl)propanamide | 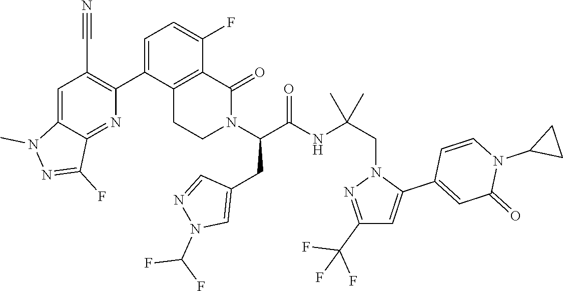 | 728.4 (M + H) |
| 58^{a} | 2-(5-(6-cyano-3-fluoro- 1-methyl-1H- pyrazolo[4,3-b]pyridin- 5-yl)-8-fluoro-1-oxo- 3,4-dihydroisoquinolin- 2(1H)-yl)-N-(1-(5- cyclopropyl-3- (trifluoromethyl)-1H- pyrazol-1-yl)-2- methylpropan-2-yl)-3- (1-(difluoromethyl)-1H- pyrazol-4- yl)propanamide (racemic mixture) | 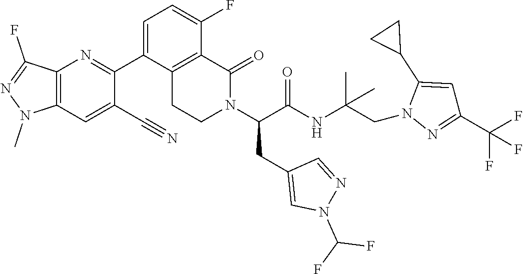 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3C3CC3)C4=O)c(C#N)cc21] | 757.2 (M + H) |
| 59^{b} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-N-(1-(5-(1- cyclopropyl-2-oxo-1,2- dihydropyrimidin-4-yl)- 3-(trifluoromethyl)-1H- pyrazol-1-yl)-2- methylpropan-2-yl)-3- (1-(difluoromethyl)-1H- pyrazol-4- yl)propanamide | 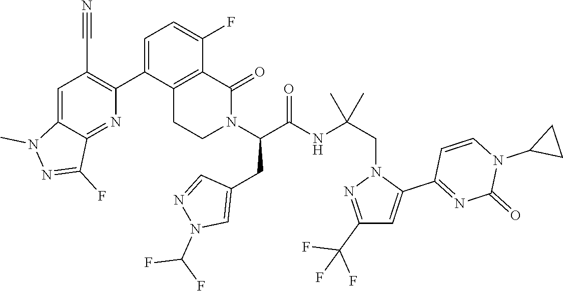 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccn(C5CC5)c(=O)n3)C4=O)c(C#N)cc21] | 851.2 (M + H) |
| 60 | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(1-(2- (2,4-dioxoimidazolidin- 1-yl)-4-fluorophenyl)-2- methylpropan-2- yl)propanamide | 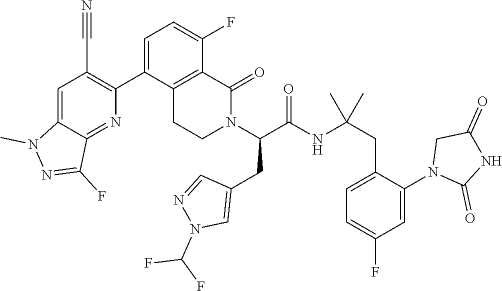 [SMILES: Cn1nc(F)c2nc(-c3ccc(F)c4c3CCN([C@H](Cc3cnn(C(F)F)c3)C(=O)NC(C)(C)Cc3ccc(F)cc3N3CC(=O)NC3=O)C4=O)c(C#N)cc21] | 775.2 (M + H) |
| 61^{c} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(1-(4- fluoro-2-(1-methyl-6- oxo-1,6- dihydropyrimidin-4- yl)phenyl)-2- methylpropan-2- yl)propanamide | 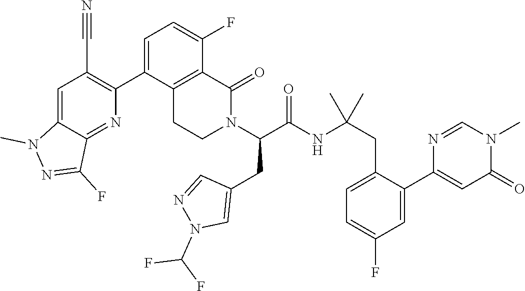 [SMILES: Cn1cnc(-c2cc(F)ccc2CC(C)(C)NC(=O)[C@@H](Cc2cnn(C(F)F)c2)N2CCc3c(-c4nc5c(F)nn(C)c5cc4C#N)ccc(F)c3C2=O)cc1=O] | 785.2 (M + H) |
| 62^{d} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-N-(1-(1-(1- cyclopropyl-2-oxo-1,2- dihydropyridin-4-yl)-4- (trifluoromethyl)-1H- imidazol-2-yl)-2- methylpropan-2-yl)-3- (1-(difluoromethyl)-1H- pyrazol-4- yl)propanamide | 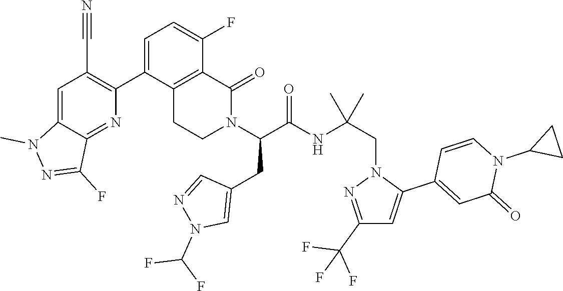 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccn(C5CC5)c(=O)c3)C4=O)c(C#N)cc21] | 850.2 (M + H) |
| 63^{e} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(5-methyl-3- (trifluoromethyl)-1H- pyrazol-1-yl)propan-2- yl)propanamide | 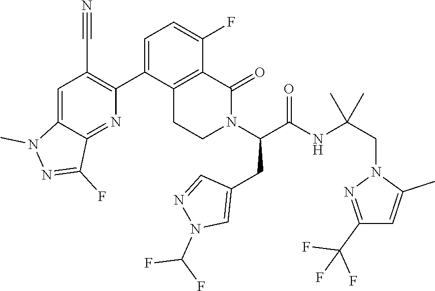 | 731.1 (M + H) |
| 64 | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(1-(2- (methylamino)-2- oxoethyl)-4- (trifluoromethyl)-1H- imidazol-2-yl)propan-2- yl)propanamide | 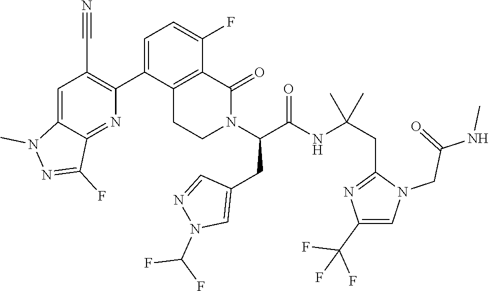 | 788.8 (M + H) |
| 65 | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(1-(2- morpholino-2-oxoethyl)- 4-(trifluoromethyl)-1H- imidazol-2-yl)propan-2- yl)propanamide | 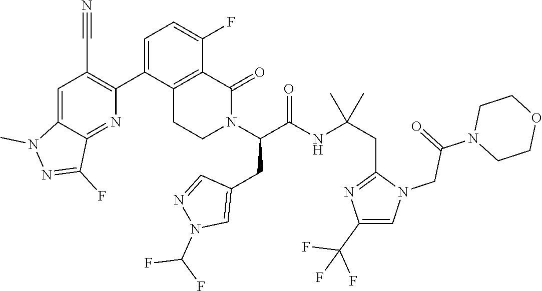 [SMILES: Cn1nc(F)c2nc(-c3ccc(F)c4c3CCN([C@H](Cc3cnn(C(F)F)c3)C(=O)NC(C)(C)Cc3nc(C(F)(F)F)cn3CC(=O)N3CCOCC3)C4=O)c(C#N)cc21] | 844.6 (M + H) |
| 66 | (R)-N-(1-(5-bromo-3- (trifluoromethyl)-1H- pyrazol-1-yl)-2- methylpropan-2-yl)-2- (5-(6-cyano-3-fluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide | 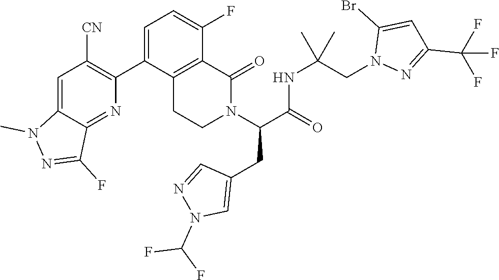 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3Br)C4=O)c(C#N)cc21] | (⁷⁹Br/⁸¹Br) 795.2/797.2 (M + H) |

^{a}Final purification by SFC, mobile phase: 35% EtOH (0.2% IPAm)/65% CO2 on (S,S) Whelk-01, the compound was obtained as the first eluting isomer
^{b}Final purification by SFC, mobile phase: [A: CO2; B: EtOH (0.1% NH3H2O)]; B %: 40.00%-40.00% on Daicel Chiralpak IM, the compound was
obtained as the second eluting isomer
^{c}Final purification by SFC, mobile phase: 35% EtOH (0.2% IPAm)/65% CO2 on (S,S) Whelk-01, the compound was obtained as the first eluting isomer
^{d}Final purification by SFC, mobile phase: [A: CO2; B: EtOH (0.1% NH3H2O)]; B %: 40.00%-40.00% on Daicel Chiralpak IM, the compound was
obtained as the main eluting isomer
^{e}Final purification by SFC, mobile phase: [A: CO2; B: EtOH (0.1% NH3H2O)]; B %: 45.00%-45.00%, on (S,S) Whelk-01, the compound was obtained
as the first eluting isomer

### Example 67

### (2R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(1-(1-methyl-2-oxopyrrolidin-3-yl)-4-(trifluoromethyl)-1H-imidazol-2-yl)propan-2-yl)propanamide (isomer 2)

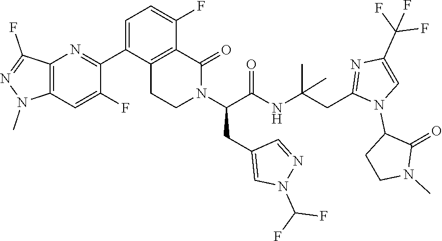 [SMILES: CN1CCC(n2cc(C(F)(F)F)nc2CC(C)(C)NC(=O)[C@@H](Cc2cnn(C(F)F)c2)N2CCc3c(-c4nc5c(F)nn(C)c5cc4F)ccc(F)c3C2=O)C1=O]

To a mixture of rac-(R)-3-(2-(2-amino-2-methylpropyl)-4-(trifluoromethyl)-1H-imidazol-1-yl)-1-methylpyrrolidin-2-one hydrochloride (163.4 mg, 1.5 eq, 479.5 μmol), (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (162.4 mg, 0.9762 eq, 312.1 μmol) and diethyl (4-oxo-1,2,3-benzotriazin-3-yl)phosphate (211.2 mg, 2.208 eq, 705.8 μmol) in DMF (4 mL) was added 4-methylmorpholine (0.14 g, 0.15 mL, 4.3 eq, 1.4 mmol). The reaction was stirred under N₂ atmosphere at RT for 2 h. Then, the reaction was quenched by the addition of formic acid (150 μL) and purified by reverse phase column chromatography (C18, 0-100% ACN/10 mM ammonium bicarbonate with 5% MeOH gradient) to afford a diastereomeric mixture of the title compound (158.2 mg, 100% pure, 61% yield) as a white solid. The mixture underwent chiral separation by SFC (column: (S,S) Welk-01, (21×250 mm, 10 μm); mobile phase: 35% IPA (0.5% DMEA): 65% CO₂; flow rate: 80 mL/min). The second eluting peak afforded the title compound (60.7 mg, 100% purity, >99% e.e., 24% yield) as an off-white solid. ES-MS m/z 807.5 (M+H).

The compounds in the following table were prepared in similar manner as described in Example 67 using the appropriate carboxylic acid and primary amine prepared above. Varying reaction temperatures, reaction time and, further purification by supercritical fluid chromatography (SFC) can be used to increase purity as desired and apparent to one skilled in the art.

TABLE 53

| | | | ES-MS |
|---|---|---|---|
| Example | Chemical Name | Structure | m/z |
| 68^{a} | (R)-N-(1-(5-(1- cyclobutyl-2-oxo-1,2- dihydropyridin-4-yl)-3- (trifluoromethyl)-1H- pyrazol-1-yl)-2- methylpropan-2-yl)-2- (5-(3,6-difluoro-1-methyl- 1H-pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide | 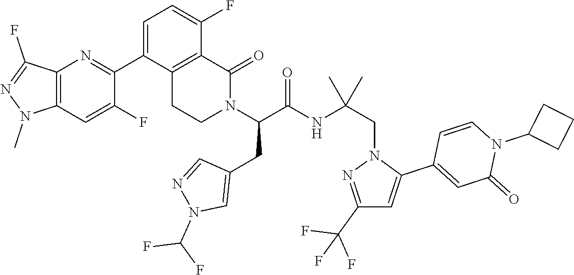 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccn(C5CCC5)c(=O)c3)C4=O)c(F)cc21] | 857.4 (M + H) |
| 69 | (2R)-N-(1-(1-(1- cyclopropyl-2- oxopyrrolidin-3-yl)-4- (trifluoromethyl)-1H- imidazol-2-yl)-2- methylpropan-2-yl)-2- (5-(3,6-difluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide (mixture of isomers) | 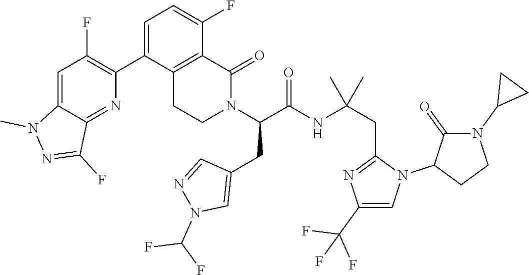 [SMILES: Cn1nc(F)c2nc(-c3ccc(F)c4c3CCN([C@H](Cc3cnn(C(F)F)c3)C(=O)NC(C)(C)Cc3nc(C(F)(F)F)cn3C3CCN(C5CC5)C3=O)C4=O)c(F)cc21] | 833.4 (M + H) |
| 70^{b} | (R)-N-(1-(1-(1- cyclopropyl-2-oxo-1,2- dihydropyridin-4-yl)-4- (trifluoromethyl)-1H- imidazol-2-yl)-2- methylpropan-2-yl)-2- (5-(3,6-difluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide | 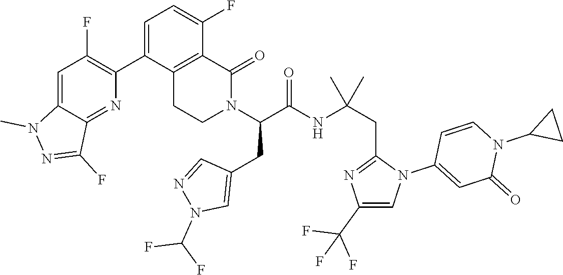 [SMILES: Cn1nc(F)c2nc(-c3ccc(F)c4c3CCN([C@H](Cc3cnn(C(F)F)c3)C(=O)NC(C)(C)Cc3nc(C(F)(F)F)cn3-c3ccn(C5CC5)c(=O)c3)C4=O)c(F)cc21] | 843.2 (M + H) |
| 71^{b} | (R)-N-(1-(5-(1- cyclopropyl-2-oxo-1,2- dihydropyrimidin-4-yl)- 3-(trifluoromethyl)-1H- pyrazol-1-yl)-2- methylpropan-2-yl)-2- (5-(3,6-difluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide | 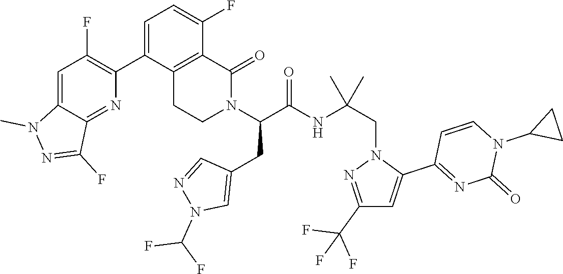 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccn(C5CC5)c(=O)n3)C4=O)c(F)cc21] | 844.2 (M + H) |
| 72^{b} | (R)-N-(1-(5- ([1,2,4]triazolo[4,3- a]pyrimidin-6-yl)-3- (trifluoromethyl)-1H- pyrazol-1-yl)-2- methylpropan-2-yl)-2- (5-(3,6-difluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide | 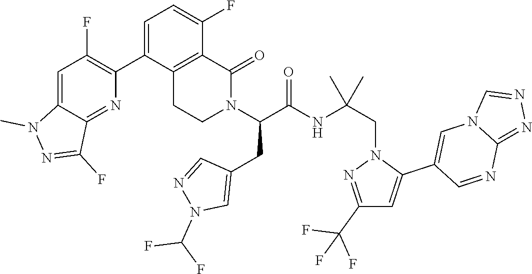 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3cnc5nncn5c3)C4=O)c(F)cc21] | 828.2 (M + H) |
| 73 | rac-N-(1-(1-(6- cyanopyridazin-3-yl)-4- (trifluoromethyl)-1H- imidazol-2-yl)-2- methylpropan-2-yl)-2- (5-(3,6-difluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4- yl)propanamide | 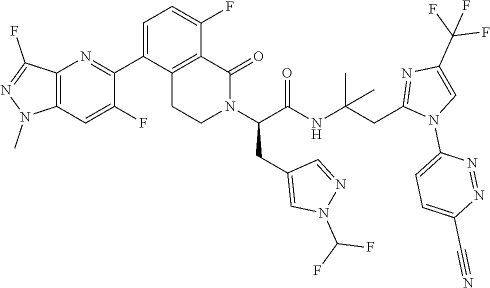 [SMILES: Cn1nc(F)c2nc(-c3ccc(F)c4c3CCN([C@H](Cc3cnn(C(F)F)c3)C(=O)NC(C)(C)Cc3nc(C(F)(F)F)cn3-c3ccc(C#N)nn3)C4=O)c(F)cc21] | 813.2 (M + H) |
| 74 | (R)-2-(5-(3,6-difluoro-1- methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(5-(6-(oxetan- 3-yl)pyridin-3-yl)-3- (trifluoromethyl)-1H- pyrazol-1-yl)propan-2- yl)propanamide | 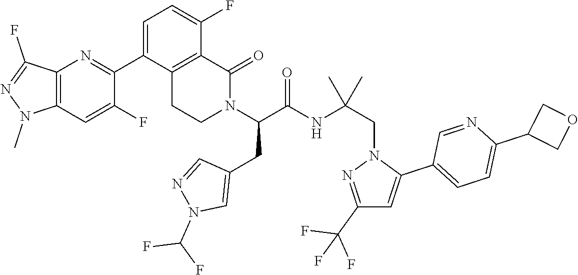 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccc(C5COC5)nc3)C4=O)c(F)cc21] | 843.4 (M + H) |
| 75 | (R)-2-(5-(3-chloro-6- cyano-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(1-((1-methyl- 1H-pyrazol-3- yl)methyl)-4- (trifluoromethyl)-1H- imidazol-2-yl)propan-2- yl)propanamide | 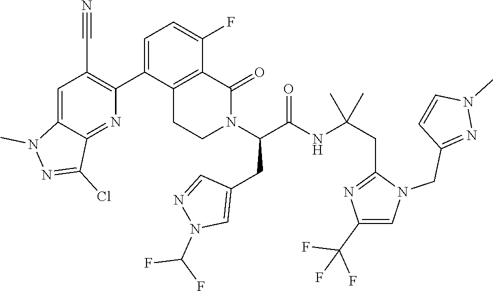 | 827.2 (M + H) |
| 76^{b} | (R)-2-(5-(3-chloro-6- cyano-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(1-(5- (2- ((dimethylamino)methyl) pyrimidin-5-yl)-3- (trifluoromethyl)-1H- pyrazol-1-yl)-2- methylpropan-2- yl)propanamide | 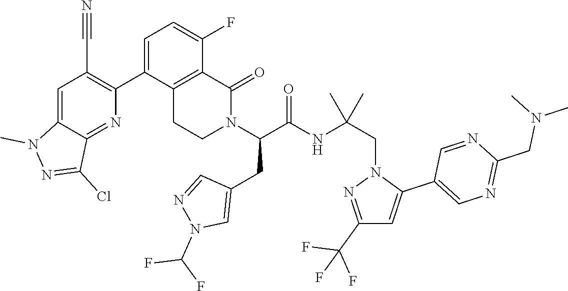 | 868.1 (M + H) |
| 77 | (R)-2-(5-(3-chloro-6- cyano-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(1-methyl-4- (trifluoromethyl)-1H- imidazol-2-yl)propan-2- yl)propanamide | 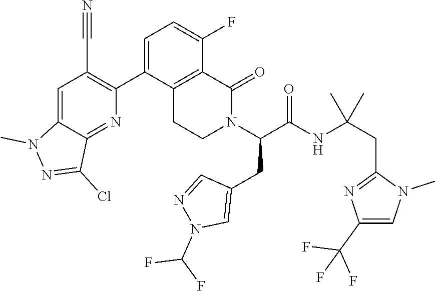 | 747.2 (M + H) |
| 78^{b} | (R)-2-(5-(3,6-difluoro-1- (methyl-d3)-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(5-(1-methyl- d3)-6-oxo-1,6- dihydropyridin-3-yl)-3- (trifluoromethyl)-1H- pyrazol-1-yl)propan-2- yl)propanamide | 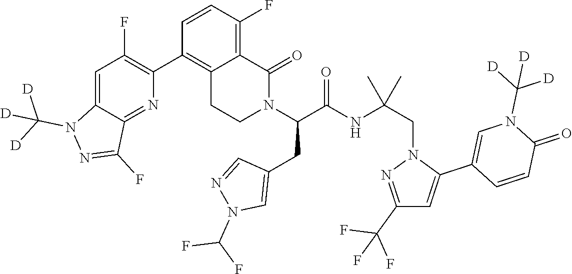 [SMILES: [2H]C([2H])([2H])n1cc(-c2cc(C(F)(F)F)nn2CC(C)(C)NC(=O)[C@@H](Cc2cnn(C(F)F)c2)N2CCc3c(-c4nc5c(F)nn(C([2H])([2H])[2H])c5cc4F)ccc(F)c3C2=O)ccc1=O] | 823.3 (M + H) |
| 79^{b} | (R)-2-(5-(3-chloro-6- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(1-(5- (1-(difluoromethyl)-2- oxo-1,2-dihydropyridin- 4-yl)-3- (trifluoromethyl)-1H- pyrazol-1-yl)-2- methylpropan-2- yl)propanamide | 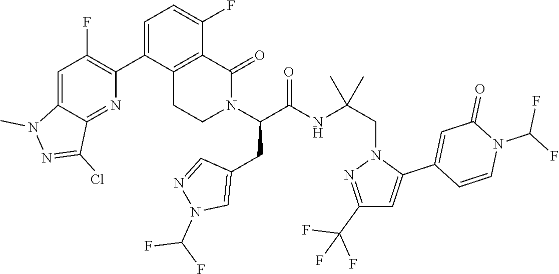 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3-c3ccn(C(F)F)c(=O)c3)C4=O)c(F)cc21] | 869.0 (M + H) |
| 80 | (R)-3-(1- (difluoromethyl)-1H- pyrazol-4-yl)-2-(8- fluoro-5-(4-methoxy-1- methyl-6-oxo-1,6- dihydropyridazin-3-yl)- 1-oxo-3,4- dihydroisoquinolin- 2(1H)-yl)-N-(1-(5-(2-(2- hydroxypropan-2- yl)pyridin-4-yl)-3- (trifluoromethyl)-1H- pyrazol-1-yl)-2- methylpropan-2- yl)propanamide | 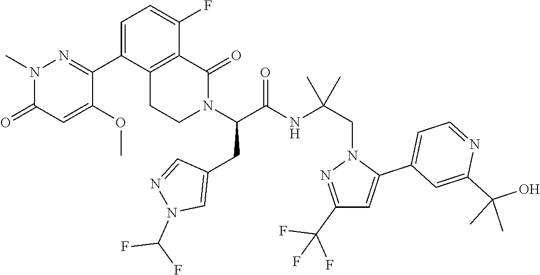 [SMILES: COc1cc(=O)n(C)nc1-c1ccc(F)c2c1CCN([C@H](Cc1cnn(C(F)F)c1)C(=O)NC(C)(C)Cn1nc(C(F)(F)F)cc1-c1ccnc(C(C)(C)O)c1)C2=O] | 816.2 (M + H) |
| 81 | (R)-3-(1- (difluoromethyl)-1H- pyrazol-4-yl)-2-(8- fluoro-5-(4-methoxy-1- methyl-6-oxo-1,6- dihydropyridazin-3-yl)- 1-oxo- 3,4-dihydroisoquinolin- 2(1H)-yl)-N-(1-(5-(5- hydroxypyridin-2-yl)-3- (trifluoromethyl)-1H- pyrazol-1-yl)-2- methylpropan-2- yl)propanamide | 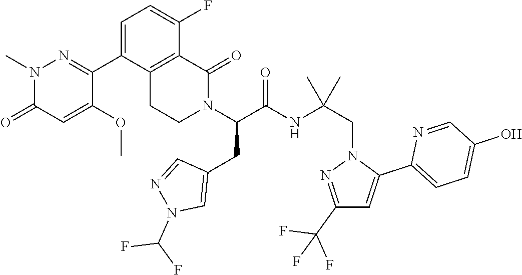 [SMILES: COc1cc(=O)n(C)nc1-c1ccc(F)c2c1CCN([C@H](Cc1cnn(C(F)F)c1)C(=O)NC(C)(C)Cn1nc(C(F)(F)F)cc1-c1ccc(O)cn1)C2=O] | 774.2 (M + H) |
| 82^{b} | (R)-1-(2-(3-(1- (difluoromethyl)-1H- pyrazol-4-yl)-2-(8- fluoro-5-(6-fluoro-1- methyl-1H- pyrazolo[4,3-b]pyridin- 5-yl)-1-oxo-3,4- dihydroisoquinolin- 2(1H)-yl)propanamido)- 2-methylpropyl)-N,N- dimethyl-3- (trifluoromethyl)-1H- pyrazole-5-carboxamide | 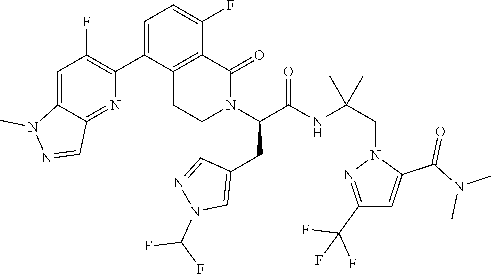 | 763.1 (M + H) |
| 83^{b} | (R)-N-(1-(5-(1- cyclopropyl-2-oxo-1,2- dihydropyridin-4-yl)-3- (trifluoromethyl)-1H- pyrazol-1-yl)-2- methylpropan-2-yl)-3- (1-(difluoromethyl)-1H- pyrazol-4-yl)-2-(8- fluoro-5-(5-fluoro-2- methoxypyrimidin-4-yl)- 1-oxo- 3,4-dihydroisoquinolin- 2(1H)-yl)propanamide | 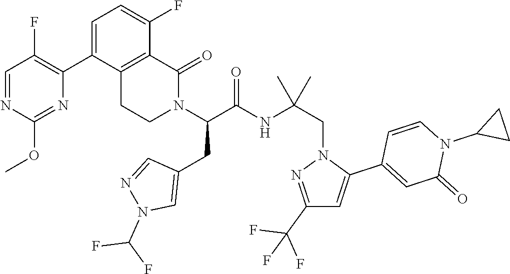 [SMILES: COc1ncc(F)c(-c2ccc(F)c3c2CCN([C@H](Cc2cnn(C(F)F)c2)C(=O)NC(C)(C)Cn2nc(C(F)(F)F)cc2-c2ccn(C4CC4)c(=O)c2)C3=O)n1] | 802.2 (M + H) |
| 84^{b} | (R)-2-(3-chloro-6- cyano-1-methyl-1H- pyrazolo[4,3-b]pyridin- 5-yl)-8-fluoro-1-oxo- 3,4-dihydroisoquinolin- 2(1H)-yl)-N-(1-(5-(2- cyclopropylpyrimidin-5- yl)-3-(trifluoromethyl)- 1H-pyrazol-1-yl)-2- methylpropan-2-yl)-3- (1-methyl-1H-pyrazol-4- yl)propanamide | 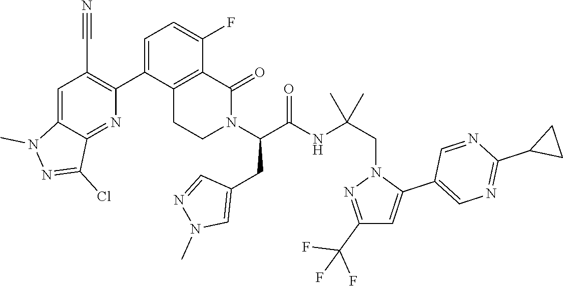 [SMILES: Cn1cc(C[C@H](C(=O)NC(C)(C)Cn2nc(C(F)(F)F)cc2-c2cnc(C3CC3)nc2)N2CCc3c(-c4nc5c(Cl)nn(C)c5cc4C#N)ccc(F)c3C2=O)cn1] | 815.2 (M + H) |
| 85^{d} | (2R)-2-(5-(5-cyano-3- fluoro-6-methylpyridin- 2-yl)-8-fluoro-1-oxo- 3,4-dihydroisoquinolin- 2(1H)-yl)-3-(1- (difluoromethyl)-1H- pyrazol-4-yl)-N-(2- methyl-1-(1-(1-methyl- 2-oxopyrrolidin-3-yl)-4- (trifluoromethyl)-1H- imidazol-2-yl)propan-2- yl)propanamide (isomer 1) | 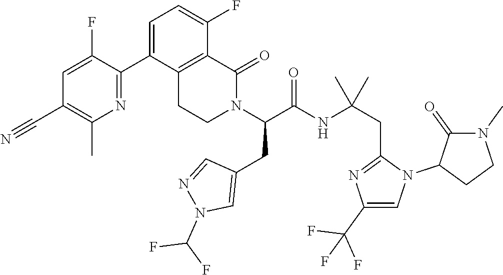 [SMILES: Cc1nc(-c2ccc(F)c3c2CCN([C@H](Cc2cnn(C(F)F)c2)C(=O)NC(C)(C)Cc2nc(C(F)(F)F)cn2C2CCN(C)C2=O)C3=O)c(F)cc1C#N] | 774.2 (M + H) |

^{a}HATU and TEA used for amide coupling
^{b}Final purification by SFC
^{d}column: DAICEL CHIRALPAK IG (250 mm*30 mm, 10 μm); mobile phase: [A: CO₂; B: EtOH (0.1% NH₃H₂O)]; B %: 35.00%-35.00%, 7.00 min; flow
rate: 80.00 g/min. First-eluting peak.

### Example 86

### (R)-2-(5-(3-chloro-6-methoxy-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide

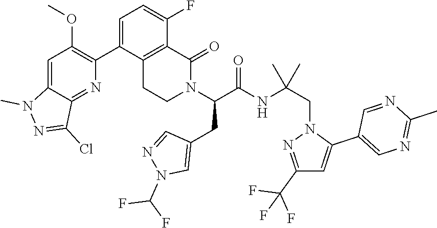

To a solution of 3,5-dichloro-6-methoxy-1-methyl-1H-pyrazolo[4,3-b]pyridine (25.2 mg, 90% wt, 1 eq, 97.7 μmol) in 1,4-dioxane (1 mL) was added (R)-3-(1-(difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)propanamide (86.3 mg, 95% wt, 1.10 eq, 108 μmol), Na₂CO₃ (21.2 mg, 99% wt, 2.03 eq, 198 μmol), 1,1′-bis(di-t-butylphosphino)ferrocene palladium dichloride (7.5 mg, 99% wt, 0.12 eq, 11 μmol) and H₂O (0.1 mL). The reaction was purged and backfilled with N₂×3 then, stirred at 90° C. for 1 h. The reaction was cooled to RT and filtered, washing the filter cake with EtOAc (10 mL×3). The filtrate was concentrated under reduced pressure. The residue was purified by column chromatography (SiO₂, 0-3% MeOH/DCM gradient) followed by prep-HPLC (Column: YMC Triart C18 250*30 mm*5 μm; mobile phase: H₂O (0.225% FA)-ACN; B %: 40%-70%, 10 min) to afford the title compound (27.5 mg, 99% purity, 33% yield) as a white solid. ES-MS m/z 830.1/831.1 (M+H).

The compounds in the following table were prepared in similar manner as described in Example 86 using the appropriate heteroaryl halide and boronic ester or boronic acid prepared above. Varying reaction temperatures are commonly in the range of 70 to 90° C. and reaction times are commonly 1 to 3 h. These variations would be apparent to one skilled in the art.

TABLE 54

| Ex- | | | ES-MS |
|---|---|---|---|
| ample | Chemical Name | Structure | m/z |
| 87^{a,i,j} | (R)-3-(1- (difluoromethyl)-1H- pyrazol-4-yl)-2-(8- fluoro-5-(3-fluoro-6- methoxy-1-methyl-1H- pyrazolo[4,3-b]pyridin- 5-yl)-1-oxo-3,4- dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)propanamide | 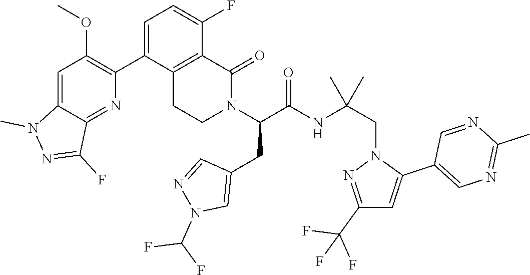 | 814.2 (M + H) |
| 88^{b,k} | (R)-2-(5-(5-chloro-2- methoxypyrimidin-4-yl)- 8-fluoro-1-oxo-3,4- dihydroisoquinolin- 2(1H)-yl)-3-(1- (difluoromethyl)-1H- pyrazol-4-yl)-N-(1-(5- (5-fluoro-1-methyl-6- oxo-1,6-dihydropyridin- 3-yl)-3- (trifluoromethyl)-1H- pyrazol-1-yl)-2- methylpropan-2- yl)propanamide | 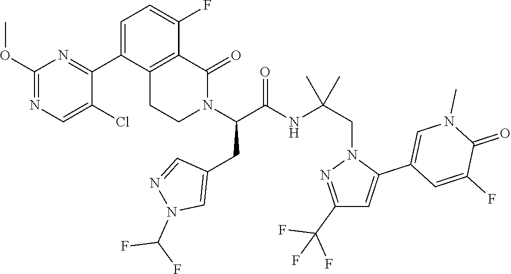 [SMILES: COc1ncc(Cl)c(-c2ccc(F)c3c2CCN([C@H](Cc2cnn(C(F)F)c2)C(=O)NC(C)(C)Cn2nc(C(F)(F)F)cc2-c2cc(F)c(=O)n(C)c2)C3=O)n1] | 810.4/ 812.4 (M + H) |
| 89^{b,h,k} | (R)-3-(1- (difluoromethyl)-1H- pyrazol-4-yl)-2-(5-(2,5- dimethoxypyrimidin-4- yl)-8-fluoro-1-oxo-3,4- dihydroisoquinolin- 2(1H)-yl)-N-(1-(5-(5- fluoro-1-methyl-6-oxo- 1,6-dihydropyridin-3- yl)-3-(trifluoromethyl)- 1H-pyrazol-1-yl)-2- methylpropan-2- yl)propanamide | 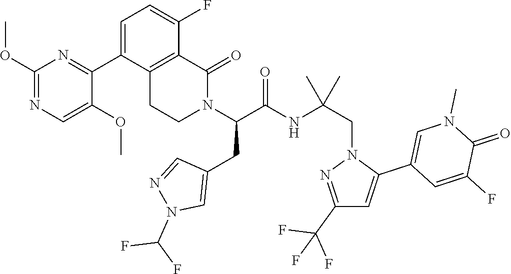 [SMILES: COc1ncc(OC)c(-c2ccc(F)c3c2CCN([C@H](Cc2cnn(C(F)F)c2)C(=O)NC(C)(C)Cn2nc(C(F)(F)F)cc2-c2cc(F)c(=O)n(C)c2)C3=O)n1] | 806.4 (M + H) |
| 90^{b,h,k} | (R)-3-(1- (difluoromethyl)-1H- pyrazol-4-yl)-N-(1-(5- (5-fluoro-1-methyl-6- oxo-1,6-dihydropyridin- 3-yl)-3- (trifluoromethyl)-1H- pyrazol-1-yl)-2- methylpropan-2-yl)-2- (8-fluoro-5-(5-fluoro-2- methoxypyrimidin-4-yl)- 1-oxo-3,4- dihydroisoquinolin- 2(1H)-yl)propanamide | 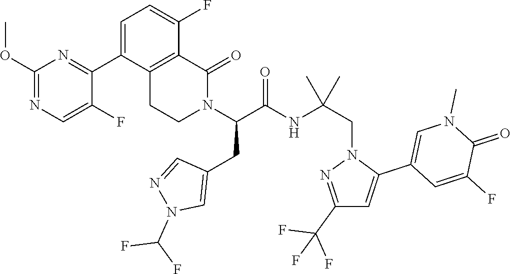 [SMILES: COc1ncc(F)c(-c2ccc(F)c3c2CCN([C@H](Cc2cnn(C(F)F)c2)C(=O)NC(C)(C)Cn2nc(C(F)(F)F)cc2-c2cc(F)c(=O)n(C)c2)C3=O)n1] | 794.4 (M + H) |
| 91^{b,h,k} | (R)-2-(5-(5-chloro-2- methylpyrimidin-4-yl)- 8-fluoro-1-oxo-3,4- dihydroisoquinolin- 2(1H)-yl)-3-(1- (difluoromethyl)-1H- pyrazol-4-yl)-N-(1-(5- (5-fluoro-1-methyl-6- oxo-1,6-dihydropyridin- 3-yl)-3- (trifluoromethyl)-1H- pyrazol-1-yl)-2- methylpropan-2- yl)propanamide | 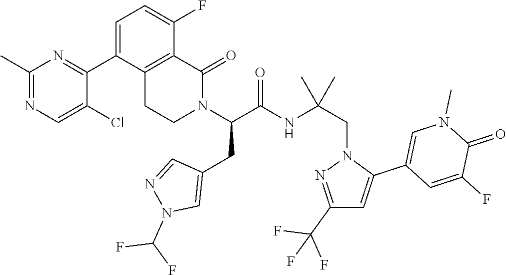 [SMILES: Cc1ncc(Cl)c(-c2ccc(F)c3c2CCN([C@H](Cc2cnn(C(F)F)c2)C(=O)NC(C)(C)Cn2nc(C(F)(F)F)cc2-c2cc(F)c(=O)n(C)c2)C3=O)n1] | 794.2 796.4 (M + H) |
| 92^{b,k} | (R)-2-(5-(5-chloro-2,6- dimethylpyrimidin-4- yl)-8-fluoro-1-oxo-3,4- dihydroisoquinolin- 2(1H)-yl)-3-(1- (difluoromethyl)-1H- pyrazol-4-yl)-N-(1-(5- (5-fluoro-1-methyl-6- oxo-1,6-dihydropyridin- 3-yl)-3- (trifluoromethyl)-1H- pyrazol-1-yl)-2- methylpropan-2- yl)propanamide | 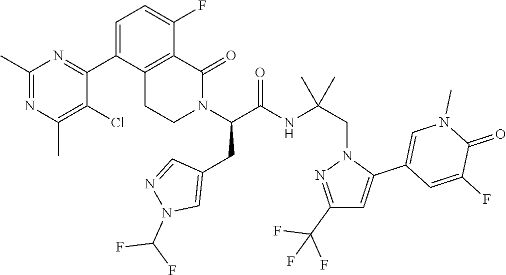 [SMILES: Cc1nc(C)c(Cl)c(-c2ccc(F)c3c2CCN([C@H](Cc2cnn(C(F)F)c2)C(=O)NC(C)(C)Cn2nc(C(F)(F)F)cc2-c2cc(F)c(=O)n(C)c2)C3=O)n1] | 808.4, 810.4 (M + H) |
| 93^{b,h,k} | (R)-3-(1- (difluoromethyl)-1H- pyrazol-4-yl)-N-(1-(5- (5-fluoro-1-methyl-6- oxo-1,6-dihydropyridin- 3-yl)-3- (trifluoromethyl)-1H- pyrazol-1-yl)-2- methylpropan-2-yl)-2- (8-fluoro-5-(5-methoxy- 2-methylpyrimidin-4- yl)-1-oxo-3,4- dihydroisoquinolin- 2(1H)-yl)propanamide | 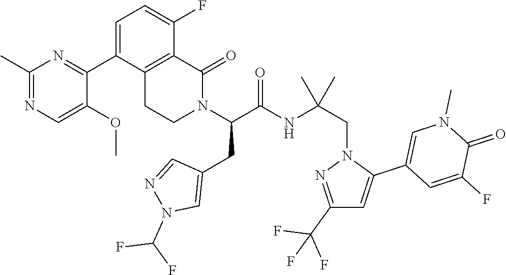 [SMILES: COc1cnc(C)nc1-c1ccc(F)c2c1CCN([C@H](Cc1cnn(C(F)F)c1)C(=O)NC(C)(C)Cn1nc(C(F)(F)F)cc1-c1cc(F)c(=O)n(C)c1)C2=O] | 790.4 (M + H) |
| 94^{c,h,l} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)-3-(1-(methyl-d3)- 1H-pyrazol-4- yl)propanamide | 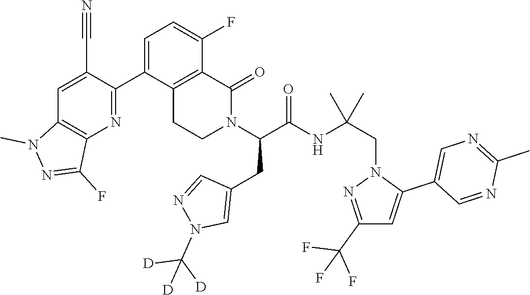 | 776.4 (M + H) |
| 95^{d,h,l} | (R)-2-(5-(3,6-difluoro-1- methyl-1H- pyrazolo[4,3-b]pyridin- 5-yl)-8-fluoro-1-oxo- 3,4-dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)-3-(1-(methyl-d3)- 1H-pyrazol-4- yl)propanamide | 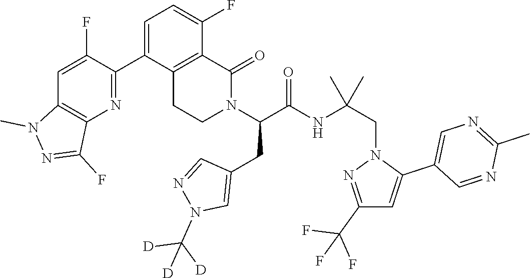 | 769.4 (M + H) |
| 96^{e.m.n} | (R)-2-(8-(3-chloro-6- cyano-1-methyl-1H- pyrazolo[4,3-b]pyridin- 5-yl)-5-fluoro-4- oxoquinazolin-3(4H)- yl)-3-(1- (difluoromethyl)-1H- pyrazol-4-yl)-N-(2- methyl-1-(5-(2- methylpyrimidin-5-yl)- 3-(trifluoromethyl)-1H- pyrazol-1-yl)propan-2- yl)propanamide | 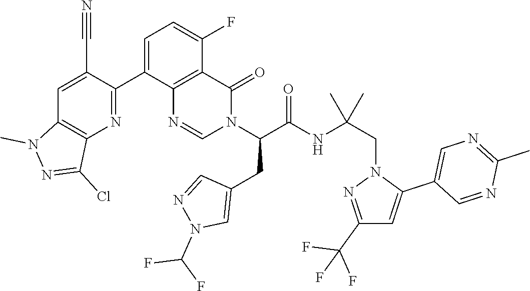 | 824.2, 826.2 (M + H) |
| 97^{a,i,j} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-N-(1-(5-(1- (difluoromethyl)-2-oxo- 1,2-dihydropyridin-4- yl)-3-(trifluoromethyl)- 1H-pyrazol-1-yl)-2- methylpropan-2-yl)-3- (6-methylpyridin-3- yl)propanamide | 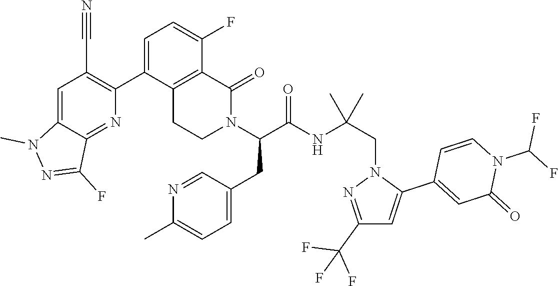 [SMILES: Cc1ccc(C[C@H](C(=O)NC(C)(C)Cn2nc(C(F)(F)F)cc2-c2ccn(C(F)F)c(=O)c2)N2CCc3c(-c4nc5c(F)nn(C)c5cc4C#N)ccc(F)c3C2=O)cn1] | 835.2 (M + H) |
| 98^{c,h,o} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-3- (imidazo[1,2- a]pyrimidin-3-yl)-N-(2- methyl-1-(5-(2- methylpyrimidin-5-yl)- 3-(trifluoromethyl)-1H- pyrazol-1-yl)propan-2- yl)propanamide | 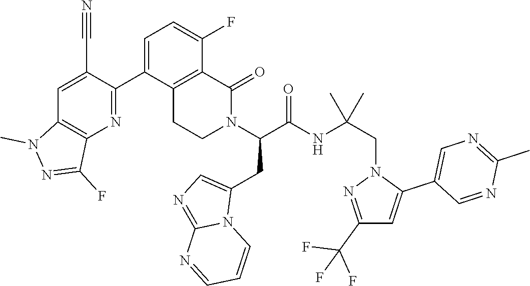 | 810.4 (M + H) |
| 99^{a,m,p} | (R)-2-(5-(3,6-difluoro-1- methyl-1H- pyrazolo[4,3-b]pyridin- 5-yl)-8-fluoro-1-oxo- 3,4-dihydroisoquinolin- 2(1H)-yl)-3- (imidazo[1,2- a]pyrimidin-3-yl)-N-(2- methyl-1-(5-(2- methylpyrimidin-5-yl)- 3-(trifluoromethyl)-1H- pyrazol-1-yl)propan-2- yl)propanamide | 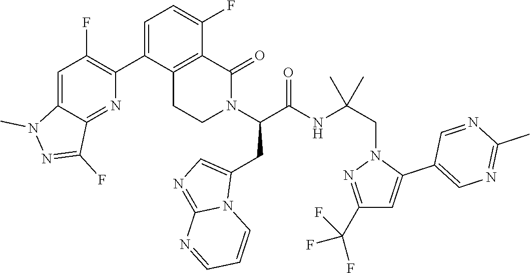 | 803.2 (M + H) |
| 100^{c,h,q} | (R)-2-(8-fluoro-5-(6- fluoro-1-methyl-1H- pyrazolo[4,3-b]pyridin- 5-yl)-1-oxo-3,4- dihydroisoquinolin- 2(1H)-yl)-3- (imidazo[1,2- a]pyrimidin-3-yl)-N-(2- methyl-1-(5-(2- methylpyrimidin-5-yl)- 3-(trifluoromethyl)-1H- pyrazol-1-yl)propan-2- yl)propanamide | 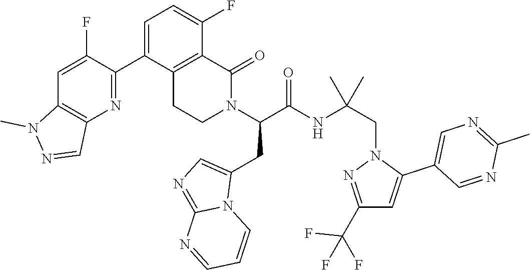 | 785.4 (M + H) |
| 101^{c,h,o} | (R)-2-(8-fluoro-5-(5- fluoro-1-methyl-1H- pyrazolo[3,4-b]pyridin- 6-yl)-1-oxo-3,4- dihydroisoquinolin- 2(1H)-yl)-3- (imidazo[1,2- a]pyrimidin-3-yl)-N-(2- methyl-1-(5-(2- methylpyrimidin-5-yl)- 3-(trifluoromethyl)-1H- pyrazol-1-yl)propan-2- yl)propanamide | 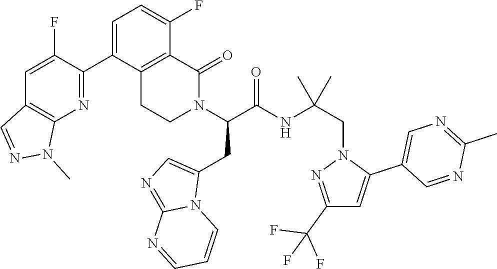 [SMILES: Cc1ncc(-c2cc(C(F)(F)F)nn2CC(C)(C)NC(=O)[C@@H](Cc2cnc3ncccn23)N2CCc3c(-c4nc5c(cnn5C)cc4F)ccc(F)c3C2=O)cn1] | 785.4 (M + H) |
| 102^{a,i,j} | (R)-2-(5-(6- (difluoromethoxy)-3- fluoro-1-methyl-1H- pyrazolo[4,3-b]pyridin- 5-yl)-8-fluoro-1-oxo- 3,4-dihydroisoquinolin- 2(1H)-yl)-3-(1- (difluoromethyl)-1H- pyrazol-4-yl)-N-(2- methyl-1-(5-(1-methyl- 6-oxo-1,6- dihydropyridin-3-yl)-3- (trifluoromethyl)-1H- pyrazol-1-yl)propan-2- yl)propanamide | 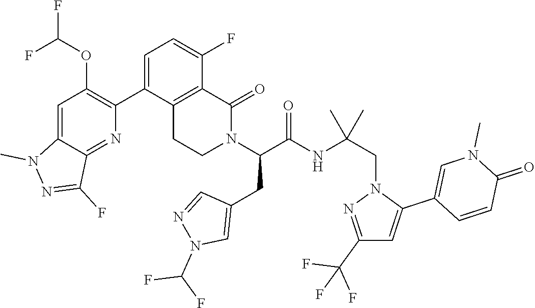 [SMILES: Cn1cc(-c2cc(C(F)(F)F)nn2CC(C)(C)NC(=O)[C@@H](Cc2cnn(C(F)F)c2)N2CCc3c(-c4nc5c(F)nn(C)c5cc4OC(F)F)ccc(F)c3C2=O)ccc1=O] | 865.1 (M + H) |
| 103^{a.m} | (R)-3-(1- (difluoromethyl)-1H- pyrazol-4-yl)-2-(8- fluoro-5-(3-fluoro-6- methoxy-1-methyl-1H- pyrazolo[4,3-b]pyridin- 5-yl)-1-oxo-3,4- dihydroisoquinolin- 2(1H)-yl)-N-(2-methyl- 1-(5-(1-methyl-6-oxo- 1,6-dihydropyridin-3- yl)-3-(trifluoromethyl)- 1H-pyrazol-1-yl)propan- 2-yl)propanamide | 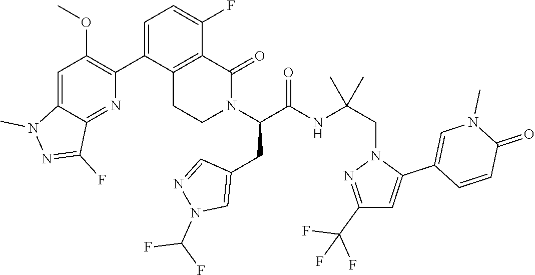 [SMILES: COc1cc2c(nc1-c1ccc(F)c3c1CCN([C@H](Cc1cnn(C(F)F)c1)C(=O)NC(C)(C)Cn1nc(C(F)(F)F)cc1-c1ccc(=O)n(C)c1)C3=O)c(F)nn2C] | 829.1 (M + H) |
| 104^{a,i,j} | (R)-3-(1- (difluoromethyl)-1H- pyrazol-4-yl)-2-(8- fluoro-5-(6-fluoro-3- methoxy-1-methyl-1H- pyrazolo[4,3-b]pyridin- 5-yl)-1-oxo-3,4- dihydroisoquinolin- 2(1H)-yl)-N-(2-methyl- 1-(5-(1-methyl-6-oxo- 1,6-dihydropyridin-3- yl)-3-(trifluoromethyl)- 1H-pyrazol-1-yl)propan- 2-yl)propanamide | 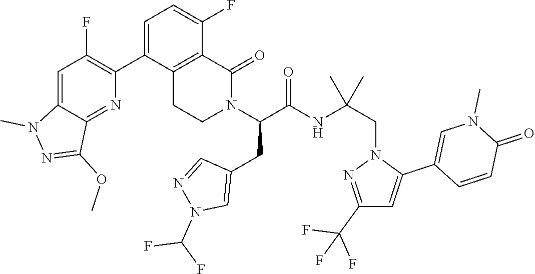 [SMILES: COc1nn(C)c2cc(F)c(-c3ccc(F)c4c3CCN([C@H](Cc3cnn(C(F)F)c3)C(=O)NC(C)(C)Cn3nc(C(F)(F)F)cc3-c3ccc(=O)n(C)c3)C4=O)nc12] | 829.4 (M + H) |
| 105^{f,h,i,j} | (R)-2-(5-(3-chloro-5- cyano-6- methoxypyridin-2-yl)-8- fluoro-1-oxo-3,4- dihydroisoquinolin- 2(1H)-yl)-3-(1- (difluoromethyl)-1H- pyrazol-4-yl)-N-(2- methyl-1-(5-(1-methyl- 6-oxo-1,6- dihydropyridin-3-yl)-3- (trifluoromethyl)-1H- pyrazol-1-yl)propan-2- yl)propanamide | 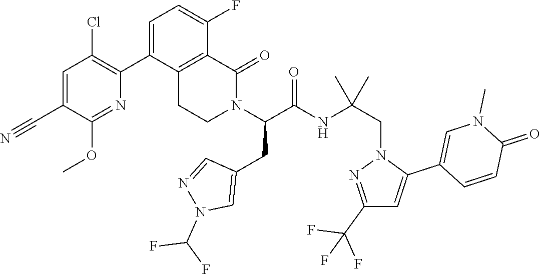 [SMILES: COc1nc(-c2ccc(F)c3c2CCN([C@H](Cc2cnn(C(F)F)c2)C(=O)NC(C)(C)Cn2nc(C(F)(F)F)cc2-c2ccc(=O)n(C)c2)C3=O)c(Cl)cc1C#N] | 816.2, 818.1 (M + H) |
| 106^{g,h,i,j} | (R)-2-(5-(3,5-dicyano-6- methylpyridin-2-yl)-8- fluoro-1-oxo-3,4- dihydroisoquinolin- 2(1H)-yl)-3-(1- (difluoromethyl)-1H- pyrazol-4-yl)-N-(2- methyl-1-(5-(1-methyl- 6-oxo-1,6- dihydropyridin-3-yl)-3- (trifluoromethyl)-1H- pyrazol-1-yl)propan-2- yl)propanamide | 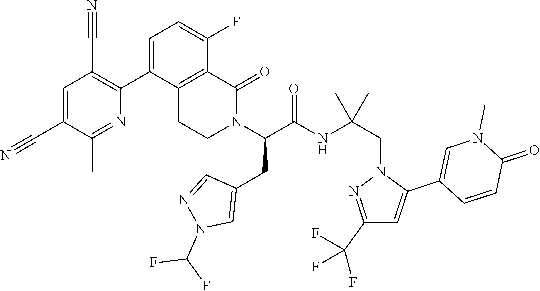 [SMILES: Cc1nc(-c2ccc(F)c3c2CCN([C@H](Cc2cnn(C(F)F)c2)C(=O)NC(C)(C)Cn2nc(C(F)(F)F)cc2-c2ccc(=O)n(C)c2)C3=O)c(C#N)cc1C#N] | 791.2 (M + H) |
| 107^{a,r,s} | (R)-3-(1- (difluoromethyl)-1H- pyrazol-4-yl)-2-(8- fluoro-5-(4-methoxy-6- oxo-1-(trifluoromethyl)- 1,6-dihydropyridin-3- yl)-1-oxo-3,4- dihydroisoquinolin- 2(1H)-yl)-N-(2-methyl- 1-(5-(1-methyl-6-oxo- 1,6-dihydropyridin-3- yl)-3-(trifluoromethyl)- 1H-pyrazol-1-yl)propan- 2-yl)propanamide | 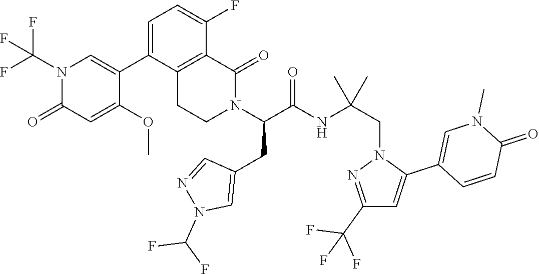 [SMILES: COc1cc(=O)n(C(F)(F)F)cc1-c1ccc(F)c2c1CCN([C@H](Cc1cnn(C(F)F)c1)C(=O)NC(C)(C)Cn1nc(C(F)(F)F)cc1-c1ccc(=O)n(C)c1)C2=O] | 841.2 (M + H) |
| 108^{b,h,k,o} | (R)-2-(5-(3-cyano-6- methylimidazo[2,1- b]thiazol-2-yl)-8-fluoro- 1-oxo-3,4- dihydroisoquinolin- 2(1H)-yl)-3-(1- (difluoromethyl)-1H- pyrazol-4-yl)-N-(2- methyl-1-(5-(1-methyl- 6-oxo-1,6- dihydropyridin-3-yl)-3- (trifluoromethyl)-1H- pyrazol-1-yl)propan-2- yl)propanamide | 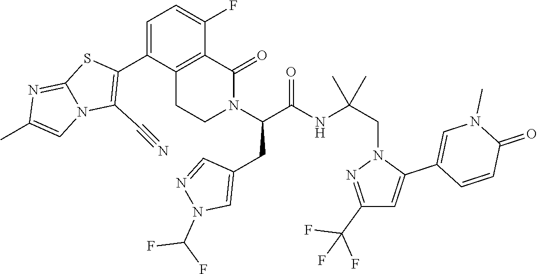 [SMILES: Cc1cn2c(C#N)c(-c3ccc(F)c4c3CCN([C@H](Cc3cnn(C(F)F)c3)C(=O)NC(C)(C)Cn3nc(C(F)(F)F)cc3-c3ccc(=O)n(C)c3)C4=O)sc2n1] | 811.4 (M + H) |
| 109^{e,i,j} | (R)-2-(3-(3-chloro-6- cyano-1-methyl-1H- pyrazolo[4,3-b]pyridin- 5-yl)-7-oxo-4,7- dihydrothieno[2,3- c]pyridin-6(5H)-yl)-3- (1-(difluoromethyl)-1H- pyrazol-4-yl)-N-(2- methyl-1-(5-(1-methyl- 6-oxo-1,6- dihydropyridin-3-yl)-3- (trifluoromethyl)-1H- pyrazol-1-yl)propan-2- yl)propanamide | 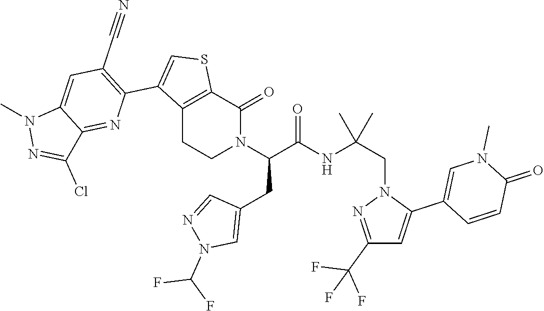 [SMILES: Cn1cc(-c2cc(C(F)(F)F)nn2CC(C)(C)NC(=O)[C@@H](Cc2cnn(C(F)F)c2)N2CCc3c(-c4nc5c(Cl)nn(C)c5cc4C#N)csc3C2=O)ccc1=O] | 828.1 830.0 (M + H) |
| 110^{a,i,p} | (R)-2-(5-(3,6-difluoro-1- methyl-1H- pyrazolo[4,3-b]pyridin- 5-yl)-8-fluoro-1-oxo- 3,4-dihydroisoquinolin- 2(1H)-yl)-N-(2-methyl- 1-(5-(2- methylpyrimidin-5-yl)- 3-(trifluoromethyl)-1H- pyrazol-1-yl)propan-2- yl)-3-(7-methyl-8-oxo- 7,8-dihydroimidazo[1,2- a]pyrazin-3- yl)propanamide | 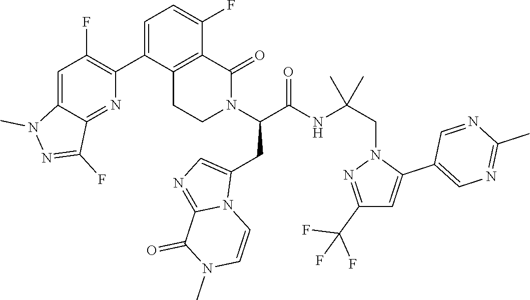 | 833.1 (M + H) |
| 111^{a,t,p} | (R)-2-(5-(6-cyano-3- fluoro-1-methyl-1H- pyrazolo[4,3-b]pyridin- 5-yl)-8-fluoro-1-oxo- 3,4-dihydroisoquinolin- 2(1H)-yl)-3-(isothiazol- 4-yl)-N-(2-methyl-1-(5- (2-methylpyrimidin-5- yl)-3-(trifluoromethyl)- 1H-pyrazol-1-yl)propan- 2-yl)propanamide | 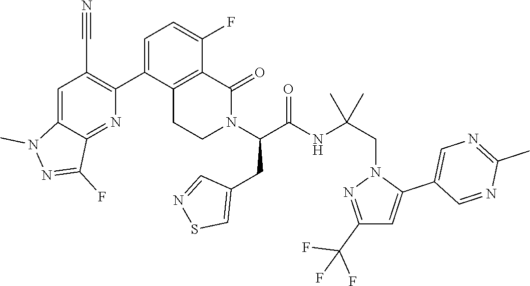 | 776.1 (M + H) |
| 112^{a,i,p,u} | (2R)-3-(1- (difluoromethyl)-1H- pyrazol-4-yl)-2-(8- fluoro-5-(5-fluoro-2- methoxypyrimidin-4-yl)- 1-oxo-3,4- dihydroisoquinolin- 2(1H)-yl)-N-(2-methyl- 1-(1-(1-methyl-2- oxopyrrolidin-3-yl)-4- (trifluoromethyl)-1H- imidazol-2-yl)propan-2- yl)propanamide (isomer 1) | 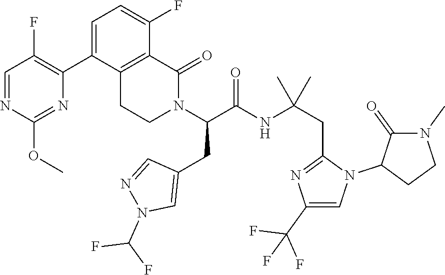 [SMILES: COc1ncc(F)c(-c2ccc(F)c3c2CCN([C@H](Cc2cnn(C(F)F)c2)C(=O)NC(C)(C)Cc2nc(C(F)(F)F)cn2C2CCN(C)C2=O)C3=O)n1] | 766.3 (M + H) |
| 113^{a,i,j,v} | (2R)-2-(5-(4-cyano-2,5- difluorophenyl)-8- fluoro-1-oxo-3,4- dihydroisoquinolin- 2(1H)-yl)-3-(1- (difluoromethyl)-1H- pyrazol-4-yl)-N-(2- methyl-1-(1-(1-methyl- 2-oxopyrrolidin-3-yl)-4- (trifluoromethyl)-1H- imidazol-2-yl)propan-2- yl)propanamide (isomer 1) | 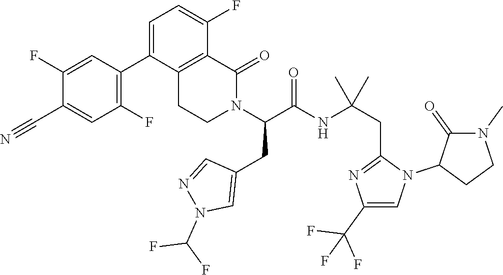 [SMILES: CN1CCC(n2cc(C(F)(F)F)nc2CC(C)(C)NC(=O)[C@@H](Cc2cnn(C(F)F)c2)N2CCc3c(-c4cc(F)c(C#N)cc4F)ccc(F)c3C2=O)C1=O] | 777.1 (M + H) |

^{a}Catalyst and base for coupling XPhos Pd G3, sodium carbonate
^{b}Catalyst and base for coupling Pd(dppf)Cl₂•CH₂Cl₂, cesium carbonate
^{c}Catalyst and base for coupling XPhos Pd G4, potassium phosphate tribasic
^{d}Catalyst and base for coupling Pd(dppf)Cl₂•CH₂Cl₂, potassium phosphate tribasic
^{e}Catalyst and base for coupling 1,1′-Bis(di-t-butylphosphino)ferrocene palladium dichloride, sodium carbonate
^{f}Catalyst and base for coupling Dichloro(tricyclohexylphosphine)palladium (II), Sodium bicarbonate
^{g}Catalyst and base for coupling XPhos Pd G3 (0.65 equiv. added in 2 portions), sodium carbonate
^{h}Reaction run overnight
ⁱPurified by silica gel chromatography using DCM/MeOH as the mobile phase
^{j}Purified by prep-HPLC-column: YMC Triart C18 250 * 30 mm * 5 μm using a mobile phase of H₂O (10 mM ammonium formate)/ACN
^{k}Purified by silica gel chromatography using 3:1 EtOAc:EtOH/heptane as the mobile phase
^{l}Purified by prep-HPLC-column: XSelect CSH C18, 50 mm × 250 mm, 5 μm, using a mobile phase of H₂O (10 mM ammonium acetate)/ACN
^{m}Purified by prep-TLC using DCM/MeOH as the mobile phase
ⁿPurified by prep-TLC using EtOAc/MeOH as the mobile phase
^{o}Purified by prep-HPLC-column: XSelect CSH C18, 30 mm × 250 mm, 5 μm, using a mobile phase of H₂O (10 mM ammonium acetate)/ACN
^{p}Purified by prep-HPLC-column: YMC Triart C18 250 * 30 mm * 5 μm using a mobile phase of H₂O (0.225% HCO₂H)/ACN
^{q}Purified by prep-HPLC-column: XSelect CSH C18, 30 mm × 150 mm, 5 μm, using a mobile phase of H₂O (0.1% HCO₂H)/ACN
^{r}Purified by prep-TLC using DCM/MeOH with 1% ammonium hydroxide as the mobile phase
^{s}Purified bv SFC-column: DAICEL CHIRALCEL OX 250 mm * 30 mm, 10 μm using a mobile phase of EtOH (0.1% ammonium hydroxide) in supercritical CO₂
^{t}Purified by silica gel chromatography using EtOAc/hexanes as the mobile phase
^{u}Diastereomers separated by SFC (column: DAICEL CHIRALPAK IG (250 mm * 30 mm, 10 μm); mobile phase: [A: CO₂; B: EtOH (0.1% NH₃ * H₂O)]; B%: 50.00%-50.00%, flow rate: 80.00 g/min), first eluting isomer.
^{v}Diastereomers separated by SFC (column: DAICEL CHIRALPAK IG (250 mm * 30 mm, 10 μm); mobile phase: [A: CO₂; B: EtOH (0.1% NH₃ * H₂O)]; B%: 60.00%-60.00%, flow rate: 80.00 g/min), first eluting isomer.

### Example 114

### (R)—N-(1-(5-cyano-3-(trifluoromethyl)-1H-pyrazol-1-yl)-2-methylpropan-2-yl)-2-(5-(6-cyano-3-fluoro-1-methyl-1H-pyrazolo[4,3-b]pyridin-5-yl)-8-fluoro-1-oxo-3,4-dihydroisoquinolin-2 (1H)-yl)-3-(1-(difluoromethyl)-1H-pyrazol-4-yl)propanamide

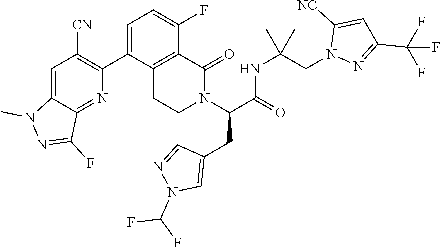 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3C#N)C4=O)c(C#N)cc21]

To a scintillation vial equipped with a red pressure relief cap and a stir bar, the following reagents were added at RT: Tetrakis(triphenylphosphine)palladium(0) (10 mg, 8.7 μmol), zinc cyanide (20 mg, 0.17 mmol), and (R)—N-(1-(5-bromo-3-(trifluoromethyl)-1H-pyrazol-1-yl)-2-methylpropan-2-yl)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4yl)propanamide (35 mg, 44 μmol), followed by dry THF (1.5 mL). The mixture was degassed by bubbling an argon stream for 3 min, then heated at 80° C. for 4 h. The reaction was allowed to cool to RT. The material was then filtered through a small pad of diatomaceous earth (approximately 2 g) using EtOAc (100 mL) as the eluent. The eluent was rinsed with a 1% w/w sodium bicarbonate aqueous solution (1 mL) to ensure removal of cyanide salts. The organic EtOAc extract was concentrated to a residue and purified by reverse phase prep-HPLC [column: C-18; mobile phase: 23 to 58% ACN in aq. NH₄HCO₃] and dried under reduced pressure to give the title compound (17 mg, 22 μmol, 50%, 96% purity). ES/MS m/z: 742.6 (M+H).

### Example 115

### (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide

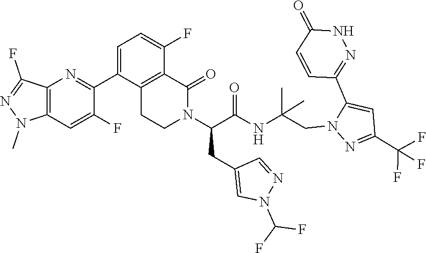 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccc(=O)[nH]n3)C4=O)c(F)cc21]

To a 20-mL vial equipped with a magnetic stir bar, was added (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (350 mg, 673 μmol), 6-(1-(2-amino-2-methylpropyl)-3-(trifluoromethyl)-1H-pyrazol-5-yl)pyridazin-3 (2H)-one dihydrochloride (254 mg, 679 μmol), and diethyl (4-oxo-1,2,3-benzotriazin-3-yl)phosphate (402 mg, 1.34 mmol). The vial was capped, purged, and backfilled with nitrogen three times. Next, DMA (3 mL) and 4-methylmorpholine (0.69 g, 0.75 mL, 6.8 mmol) were added, and the reaction mixture was stirred at RT for 4 h. After completion, the reaction mixture was combined with the mixture from another batch containing (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (50 mg, 96 μmol), and the combined mixture was diluted with water (100 mL) and extracted with EtOAc (100 mL×3). The combined organic layers were washed with saturated aqueous NaCl, dried over Na₂SO₄, filtered, and concentrated to yield a light yellow sticky solid. The solid was then subjected to reverse phase prep-HPLC [column: C-18; mobile phase: 20 to 64% ACN in aq. NH₄HCO₃]. The purified product (475 mg, 518 μmol, 77%) was obtained as white powder. For further purification, the product was subjected to chiral chromatography [Chiralpak IA column; mobile phase: 50% IPA (0.5% DMEA): 50% CO₂] to give the first eluting isomer as the title compound (375 mg) as white powder with 97% ee. ES/MS: m/z 804.2 (M+H).

### Example 116

### (R)-(3-(1-(2-(2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)-6-oxopyridazin-1 (6H)-yl)methyl dihydrogen phosphate

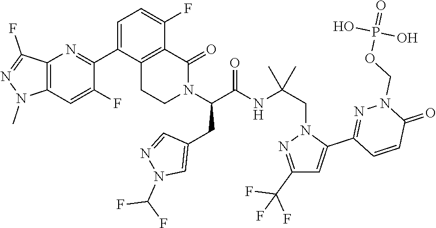 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccc(=O)n(COP(=O)(O)O)n3)C4=O)c(F)cc21]

To a 20-mL microwave vial with magnetic stir bar was added (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide (143.0 mg, 177.9 μmol) and sodium iodide (74.3 mg, 496 μmol). The vial was capped, purged and backfilled with N₂×3. Then, THF (4.5 mL) was added, and the reaction was cooled to 0° C. in an ice/water bath. Then, lithium 2-methylpropan-2-olate (26.2 mg, 327 μmol) and di-tert-butyl (chloromethyl)phosphate (123 mg, 110 μL, 474 μmol) were added and the reaction was allowed to stir at RT overnight. The reaction was quenched with water and the layers were separated. The aqueous layer was extracted with EtOAc. The combined organic layer was dried over Na₂SO₄, filtered and concentrated under reduced pressure. In another 50-mL RBF containing (R)-di-tert-butyl ((3-(1-(2-(2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)-6-oxopyridazin-1 (6H)-yl)methyl)phosphate was added a magnetic stir bar and DCM (4.448 mL, 4.5 mL) followed by 2,2,2-trifluoroacetic acid (444 mg, 300 μL, 21.9 eq, 3.89 mmol). The flask was allowed to stir at RT overnight. After completion, reaction mixture was concentrated and subjected to reverse phase prep-HPLC [column: C-18; mobile phase: 1.0 to 18% ACN with formic acid] to give the title compound (87.3 mg, 53%) as an off white solid. ES/MS m/z: 1024.4 (M−H).

### Example 117

### (2R)-3-(1-(difluoromethyl)-1H-pyrazol-4-yl)-2-(8-fluoro-5-(4-methoxy-1-methyl-6-oxo-1,6-dihydropyridazin-3-yl)-1-oxo-3,4-dihydroisoquinolin-2 (1H)-yl)-N-(2-methyl-1-(5-(1-methyl-2-oxopyrrolidin-3-yl)-3-(trifluoromethyl)-1H-pyrazol-1-yl)propan-2-yl)propanamide (isomer 1)

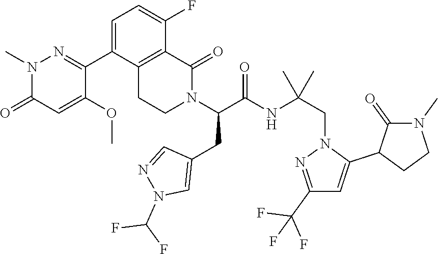

To a RBF, (R)-3-(1-(difluoromethyl)-1H-pyrazol-4-yl)-2-(8-fluoro-5-(4-methoxy-1-methyl-6-oxo-1,6-dihydropyridazin-3-yl)-1-oxo-3,4-dihydroisoquinolin-2 (1H)-yl)propanoic acid (173 mg, 352 μmol) and rac-(R)-3-(1-(2-amino-2-methylpropyl)-3-(trifluoromethyl)-1H-pyrazol-5-yl)-1-methylpyrrolidin-2-one (179 mg, 90% purity, 529 μmol) were added. The mixture was purged with nitrogen (N₂), followed by the addition of DMA (2.5 mL), 4-methylmorpholine (0.14 g, 0.15 mL, 1.4 mmol) and diethyl (4-oxo-1,2,3-benzotriazin-3-yl)phosphate (211 mg, 705 μmol) at 0° C., and the reaction mixture was allowed to slowly warm to 23° C. for 25 h. After completion, the mixture was diluted with EtOAc (30 mL) and water (30 mL). The mixture was extracted with EtOAc (3×40 mL), washed with saturated aqueous NaCl, dried over Na₂SO₄, filtered, and concentrated under reduced pressure. The residue was purified by reverse phase prep-HPLC [column: C-18; mobile phase: 15 to 48% ACN in aq. NH₄HCO₃] to give the title compound (107 mg, 138 μmol, 39%) as yellow glassy solid. For further purification, the product was subjected to chiral chromatography [Chiralpak IC column; mobile phase: 35% EtOH (0.5% DMEA): 65% CO₂ and Chiralpak AD-H column, Mobile Phase: 25% IPA (0.5% DMEA): 75% CO₂] to give the first eluting isomer (34 mg, 32%) the title compound as yellow glassy solid. ES/MS m/z: 778.4 (M+H).

### Example 118

### (R)-3-(1-(difluoromethyl)-1H-pyrazol-4-yl)-2-(9-fluoro-6-(6-fluoro-1-methyl-1H-pyrazolo[4,3-b]pyridin-5-yl)-1-oxo-1,3,4,5-tetrahydro-2H-benzo[c]azepin-2-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

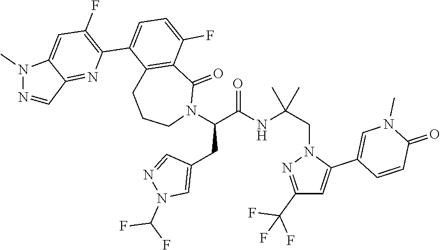 [SMILES: Cn1cc(-c2cc(C(F)(F)F)nn2CC(C)(C)NC(=O)[C@@H](Cc2cnn(C(F)F)c2)N2CCCc3c(-c4nc5cnn(C)c5cc4F)ccc(F)c3C2=O)ccc1=O]

In a N₂ filled glovebox, (R)-2-(6-chloro-9-fluoro-1-oxo-1,3,4,5-tetrahydro-2H-benzo[c]azepin-2-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (80 mg, 95% wt, 0.11 mmol), 6-fluoro-1-methyl-5-(tributylstannyl)-1H-pyrazolo[4,3-b]pyridine (81 mg, 99% wt, 0.18 mmol), CsF (130 mg, 856 μmol) and bis(di-tert-butyl (4-dimethylaminophenyl)phosphine)dichloropalladium(II) (50 mg, 71 μmol) were added into a dried 8 mL sealed reaction vial. 1,4-Dioxane (2 mL) was added subsequently. The sealed reaction vial was removed from the glovebox and the reaction mixture was stirred at 120° C. for 12 h. After completion, the reaction mixture was cooled to RT, then diluted with H₂O (20 mL) and EtOAc (20 mL). The aqueous phase was extracted with EtOAc (20 mL×2). The combined organic layers were washed with saturated aqueous NaCl (20 mL), dried over anhydrous Na₂SO₄, filtered and concentrated under reduced pressure to give a crude product. The crude product was purified by flash silica gel chromatography (eluent of 3˜5% MeOH/DCM gradient@ 30 mL/min) to give the impure product (˜30 mg). The crude product was further purified by prep-HPLC [column: C-18; mobile phase: 36 to 66% ACN in aq. NH₄HCO₃] to give the title compound (20.16 mg, 24.8 μmol, 23%) as an off-white solid. ES/MS m/z: 813.1 (M+H).

### Example 119

### 2-(7-(6-cyano-1-methyl-1H-pyrazolo[4,3-b]pyridin-5-yl)-4-fluoro-3-oxo-1,1a,3,7b-tetrahydro-2H-cyclopropa[c]isoquinolin-2-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-3-(1-methyl-1H-pyrazol-4-yl)propanamide (Diastereomer 1, Enantiomer 2)

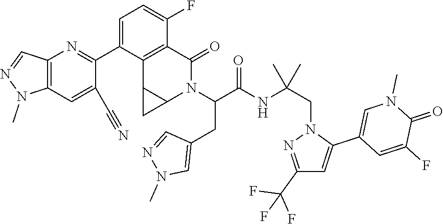 [SMILES: Cn1cc(CC(C(=O)NC(C)(C)Cn2nc(C(F)(F)F)cc2-c2cc(F)c(=O)n(C)c2)N2C(=O)c3c(F)ccc(-c4nc5cnn(C)c5cc4C#N)c3C3CC32)cn1]

A mixture of rac-(R)-2-((1aS,7bS)-7-chloro-4-fluoro-3-oxo-1,1a,3,7b-tetrahydro-2H-cyclopropa[c]isoquinolin-2-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-3-(1-methyl-1H-pyrazol-4-yl)propanamide (143.0 mg of an ˜2:1 mixture of diastereomers, 210.9 μmol), CsF (73.2 mg, 482 μmol) and XPhos-Pd-G4 (19.7 mg, 22.9 μmol) were placed under an atmosphere of argon. Then 1-methyl-5-(tributylstannyl)-1H-pyrazolo[4,3-b]pyridine-6-carbonitrile (95.7 mg, 214 μmol) was added using argon-sparged 1,4-dioxane (2 mL) to aid transfer. Stirring was initiated and the mixture was heated to 80° C. After 17.2 h, the reaction was removed from heating and combined with material from a previous pilot reaction (performed on 25 μmol scale) and the combination was filtered through a plug of diatomaceous earth using ˜50 mL of EtOAc to aid transfer and as a rinse. The filtrate was then concentrated under reduced pressure and purified by silica gel chromatography using a gradient of 0-100% of a solution of 3:1 EtOAc:EtOH in heptane followed by SFC purification to separate the diastereomers (Viridis SI 50 mm×250 mm, 5 μm using 15% MeOH in supercritical CO₂ at a flow rate of 200 ml/min). Then the separated diastereomers were each purified by chiral SFC (both on a Whelk-O1 (R,R) 30 mm×250 mm, 5 μm column using 40% MeOH with 10 mM ammonium acetate in supercritical CO₂ at a flow rate of 85 ml/min). The second eluting enantiomer from the chiral purification of the first eluting diastereomer was collected to afford the title compound (12.9 mg, 8%) as a white solid. ES-MS m/z: 800.4 (M+H).

### Example 120

### 2-(7-(6-cyano-1-methyl-1H-pyrazolo[4,3-b]pyridin-5-yl)-4-fluoro-3-oxo-1,1a,3,7b-tetrahydro-2H-cyclopropa[c]isoquinolin-2-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-3-(1-methyl-1H-pyrazol-4-yl)propanamide (Diastereoamer 2, Enantiomer 1)

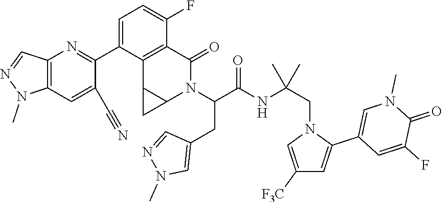 [SMILES: Cn1cc(CC(C(=O)NC(C)(C)Cn2cc(C(F)(F)F)cc2-c2cc(F)c(=O)n(C)c2)N2C(=O)c3c(F)ccc(-c4nc5cnn(C)c5cc4C#N)c3C3CC32)cn1]

The first eluting enantiomer from the chiral purification of the second eluting diastereomer of the material described in Example 119 was collected to afford the title compound (6.1 mg, 4%) as a white solid. ES-MS m/z: 800.4 (M+H)

### Example 121

### 2-(7-(6-Cyano-1-methyl-1H-pyrazolo[4,3-b]pyridin-5-yl)-4-fluoro-3-oxo-1,1a,3,7b-tetrahydro-2H-cyclopropa[c]isoquinolin-2-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-3-(1-methyl-1H-pyrazol-4-yl)propanamide (Diastereomer 2, Enantiomer 2)

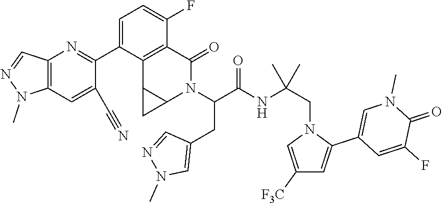 [SMILES: Cn1cc(CC(C(=O)NC(C)(C)Cn2cc(C(F)(F)F)cc2-c2cc(F)c(=O)n(C)c2)N2C(=O)c3c(F)ccc(-c4nc5cnn(C)c5cc4C#N)c3C3CC32)cn1]

The second eluting enantiomer from the chiral purification of the second eluting diastereomer of the material described in Example 119 was collected to afford the title compound (7.0 mg, 4%) as a white solid. ES-MS m/z: 800.4 (M+H).

### Example 122

### (2R)-2-(5-(6-Cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(1-(1-(p-tolyl)ethyl)-4-(trifluoromethyl)-1H-imidazol-2-yl)propan-2-yl)propanamide (Mixture of Diastereomers)

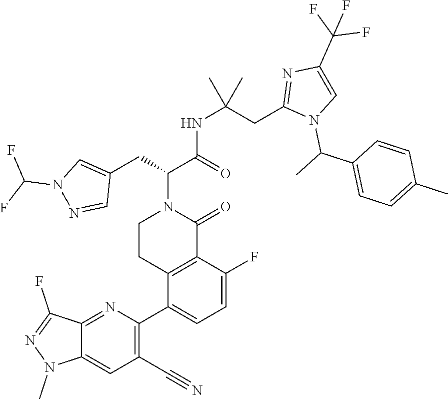 [SMILES: Cc1ccc(C(C)n2cc(C(F)(F)F)nc2CC(C)(C)NC(=O)[C@@H](Cc2cnn(C(F)F)c2)N2CCc3c(-c4nc5c(F)nn(C)c5cc4C#N)ccc(F)c3C2=O)cc1]

To a vial was added 2-methyl-1-(1-(1-(p-tolyl)ethyl)-4-(trifluoromethyl)-1H-imidazol-2-yl)propan-2-amine dihydrochloride (20 mg, 0.049 mmol) followed by a solution of (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid, dimethylethylammonium salt (21 mg, 35 μmol) and 4-methylmorpholine (15 μL, 0.14 mmol) in DMA (0.3 mL). The vial was cooled to approximately 0° C. in an ice bath, then a solution of diethyl (4-oxo-1,2,3-benzotriazin-3-yl)phosphate (21 mg, 70 μmol) in DMA (0.2 mL) was added. The mixture was allowed to slowly warm to 23° C. and stirred for 3 days. The solution was filtered through a syringe filter and concentrated under a stream of N₂. The residue was purified by reverse phase prep-HPLC [column: Phenomenex Kinetex EVO C₁₈; mobile phase: gradient of 52%-86% ACN in water (with 0.1% FA)] to give the title compound (29 mg, 99%) as a glassy solid. ES-MS m/z 835.4 (M+H).

### Example 123

### (2R)-2-(6,8-Difluoro-5-(6-fluoro-1-methyl-1H-pyrazolo[4,3-b]pyridin-5-yl)-1-oxo-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

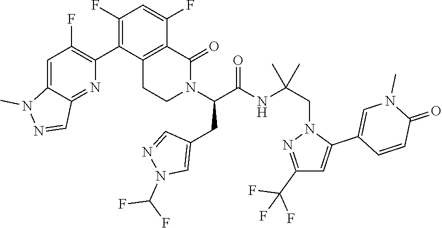 [SMILES: Cn1cc(-c2cc(C(F)(F)F)nn2CC(C)(C)NC(=O)[C@@H](Cc2cnn(C(F)F)c2)N2CCc3c(c(F)cc(F)c3-c3nc4cnn(C)c4cc3F)C2=O)ccc1=O]

To a mixture of (R)-2-(6,8-difluoro-1-oxo-5-(4,4,5,5-tetramethyl-1,3,2-dioxaborolan-2-yl)-3,4-dihydroisoquinolin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (100 mg, 87.0 μmol, 69% purity) in 1,4-dioxane (3 mL) and H₂O (0.3 mL) was added 5-bromo-6-fluoro-1-methyl-1H-pyrazolo[4,3-b]pyridine (23 mg, 97 μmol, 97% purity), Na₂CO₃ (42 mg, 4.6 Eq, 0.40 mmol) and XPhos Pd G3 (12 mg, 14 μmol). The reaction mixture was sparged with N₂, then stirred at 100° C. for 1 h. The reaction mixture was cooled to RT, combined with another batch run on 20 mg scale, filtered, and concentrated under reduced pressure. Flash chromatography (SiO₂, gradient 0-100% EtOAc in hexanes) followed by reversed-phase HPLC (YMC Triart C18, 150×25 mm, 5 μm, gradient 38-68% ACN in 10 mM aqueous NH₄HCO₃) provided the title compound (17.45 mg, 20%) as a white solid. ES-MS m/z 817.1 (M+H).

### Example 124

### (R)-3-(1-(Difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-(8-fluoro-5-(6-fluoro-1-methyl-1H-pyrazolo[4,3-b]pyridin-5-yl)-1-oxophthalazin-2 (1H)-yl)propanamide

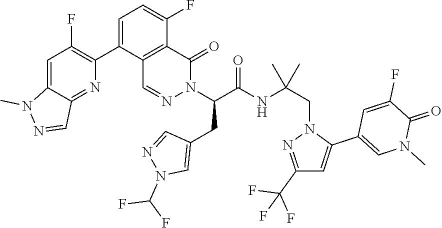 [SMILES: Cn1cc(-c2cc(C(F)(F)F)nn2CC(C)(C)NC(=O)[C@@H](Cc2cnn(C(F)F)c2)n2ncc3c(-c4nc5cnn(C)c5cc4F)ccc(F)c3c2=O)cc(F)c1=O]

A 2-dram vial was charged with (R)-2-(5-bromo-8-fluoro-1-oxophthalazin-2 (1H)-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (74.5 mg, 99.9 μmol), 5,5,5′,5′-tetramethyl-2,2′-bi (1,3,2-dioxaborinane) (34 mg, 0.15 mmol), potassium acetate (30 mg, 0.31 mmol), and Pd-117 (7.2 mg, 10 μmol). The vial was flushed with argon, then the solids were suspended in 1,4-dioxane (666 μL). The yellow mixture was sparged with argon for an additional 5 min, then the mixture was stirred at 80° C. (heating block temperature) under argon. After 2 h, the reaction mixture was cooled to RT and the crude aryl boronate solution was used in the subsequent step assuming quantitative conversion. To the crude aryl boronate mixture was added 5-bromo-6-fluoro-1-methyl-1H-pyrazolo[4,3-b]pyridine (58 mg, 0.25 mmol), XPhos Pd G4 (4.3 mg, 5.0 μmol), and a solution of potassium phosphate tribasic (32 mg, 0.15 mmol) in water (180 μL). The mixture was sparged with argon for 10 min, then the mixture was stirred at 80° C. (heating block temperature) for 20 h under argon. The reaction mixture was cooled to RT then partitioned between EtOAc (2 mL) and saturated aqueous ammonium chloride (2 mL). The layers were separated, and the aqueous layer was extracted with EtOAc (2 mL×2). The combined organic layers were stirred with SiliaMetS® Thiol (30 mg) and anhydrous sodium sulfate. The mixture was filtered through a 0.45 μm syringe filter and the filtrate concentrated under a stream of nitrogen. The residue was purified by prep-HPLC (column: XSelect CSH C18 50 mm×250 mm, 5 μm; mobile phase: A=water (10 mM ammonium acetate), B=ACN; B % 5-95%, 32 min; flow rate: 100 mL/min) to afford the title compound (38.9 mg, 47%) as a white solid. ES/MS m/z 816.2 (M+H).

### Example 125

### (R)-3-(1-(Difluoromethyl)-1H-pyrazol-4-yl)-2-(8-fluoro-5-(6-fluoro-1-methyl-1H-indazol-5-yl)-1-oxo-3,4-dihydro-2,6-naphthyridin-2(1H)-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

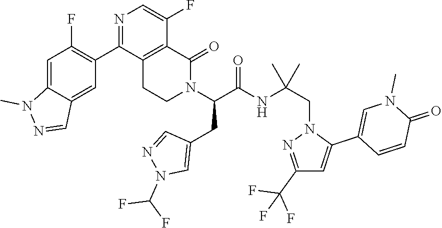 [SMILES: Cn1cc(-c2cc(C(F)(F)F)nn2CC(C)(C)NC(=O)[C@@H](Cc2cnn(C(F)F)c2)N2CCc3c(-c4cc5cnn(C)c5cc4F)ncc(F)c3C2=O)ccc1=O]

To a mixture of (R)-2-(5-chloro-8-fluoro-1-oxo-3,4-dihydro-2,6-naphthyridin-2(1H)-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (64 mg, 1 Eq, 87 μmol) and 6-fluoro-1-methyl-5-(4,4,5,5-tetramethyl-1,3,2-dioxaborolan-2-yl)-1H-indazole (42 mg, 1.5 Eq, 0.13 mmol) in 1,4-dioxane (1 mL) and H₂O (0.1 mL) was added cesium carbonate (85 mg, 3 Eq, 0.26 mmol) and XPhos Pd G3 (15 mg, 0.2 Eq, 17 μmol). The mixture was degassed and purged with N₂ 3 times. Then the reaction mixture was stirred at 100° C. for 1 h. The reaction mixture was concentrated under reduced pressure. The residue was purified by prep-TLC (20:1 DCM/MeOH, R_{f}=0.4) and further purified by prep-HPLC (column: YMC Triart C18 250×30 mm×5 mm; mobile phase: A=H₂O (10 mM NH₄HCO₃); B=ACN; B % 36-66%, 10 min; flow rate: 25 mL/min) to afford the title compound (11.02 mg, 15% yield) as a white solid. ES/MS m/z 799.1 (M+H)

### Example 126

### (R)—N-(1-(5-(5-Fluoro-1-(methyl-d3)-6-oxo-1,6-dihydropyridin-3-yl)-3-(trifluoromethyl)-1H-pyrazol-1-yl)-2-methylpropan-2-yl-1,1-d2)-2-(8-fluoro-5-(6-fluoro-1-(methyl-d3)-1H-pyrazolo[4,3-b]pyridin-5-yl)-1-oxo-3,4-dihydroisoquinolin-2(1H)-yl)-3-(1-(methyl-d3)-1H-pyrazol-4-yl)propanamide

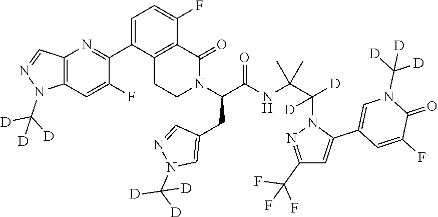 [SMILES: [2H]C([2H])([2H])n1cc(C[C@H](C(=O)NC(C)(C)C([2H])([2H])n2nc(C(F)(F)F)cc2-c2cc(F)c(=O)n(C([2H])([2H])[2H])c2)N2CCc3c(-c4nc5cnn(C([2H])([2H])[2H])c5cc4F)ccc(F)c3C2=O)cn1]

A mixture of (R)-2-(5-bromo-8-fluoro-1-oxo-3,4-dihydroisoquinolin-2 (1H)-yl)-N-(1-(5-(5-fluoro-1-(methyl-d3)-6-oxo-1,6-dihydropyridin-3-yl)-3-(trifluoromethyl)-1H-pyrazol-1-yl)-2-methylpropan-2-yl-1,1-d2)-3-(1-(methyl-d3)-1H-pyrazol-4-yl)propanamide (119.4 mg, 166.2 μmol), bis(pinacolato)diboron (84.4 mg, 332 μmol), potassium acetate (48.9 mg, 498 μmol) and [1,1′-bis(diphenylphosphino)ferrocene]dichloropalladium(II)·CH₂Cl₂ (13.6 mg, 16.6 μmol) in 1,4-dioxane (1 mL) was sparged with nitrogen then heated at 100° C. for 2 days under nitrogen. The resulting mixture was concentrated under reduced pressure to give the crude borylation intermediate. This crude residue was combined with 5-bromo-6-fluoro-1-(methyl-d3)-1H-pyrazolo[4,3-b]pyridine (57.8 mg, 248.1 μmol), cesium carbonate (135.3 mg, 415.3 μmol) and [1,1′-bis(diphenylphosphino)ferrocene]dichloropalladium(II)·CH₂Cl₂ (13.6 mg, 16.6 μmol) under an atmosphere of nitrogen and was treated with a nitrogen sparged mixture of 1,4-dioxane (0.6 mL) and water (0.06 mL). The resulting mixture was sparged with nitrogen for 2 min, then stirred in a reaction block set at 90° C. overnight. The reaction mixture was cooled to RT, diluted with EtOAc and filtered through a plug of silica gel. The filtrate was evaporated to dryness, and the residue was purified by reverse phase HPLC (C₁₈, eluting with 23-58% ACN in 0.1% FA/water) to obtain the title compound (13.4 mg, 10%) as a brown solid. ES-MS m/z 792.2 (M+H).

The compounds in the following table were prepared in similar manner as described in Example 122 using the appropriate carboxylic acid and primary amine prepared above. Varying reaction temperatures between 0° C. and RT, reaction time between 1 and 72 hours, aqueous workup with organic solvent extraction (such as EtOAc) and further purification by supercritical fluid chromatography (SFC) can be used to increase purity as desired and apparent to one skilled in the art.

TABLE 55

| Ex- | | | ES-MS |
|---|---|---|---|
| ample | Chemical Name | Structure | m/z |
| 127^{ab} | (R)-2-(5-(3-Chloro-6- fluoro-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(1-(5- (4-(dimethyl- phosphoryl)phenyl)- 3-(trifluoromethyl)- 1H-pyrazol-1-yl)-2- methylpropan-2- yl)propanamide | 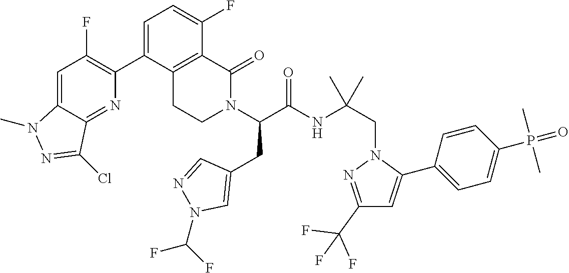 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3-c3ccc(P(C)(C)=O)cc3)C4=O)c(F)cc21] | 878.2, 880.1 (M + H) |
| 128^{ac} | (R)-2-(5-(3-Chloro-6- fluoro-1-methyl-1H- pyrazolo[4,3-b]pyridin-5- yl)-8-fluoro-1-oxo-3,4- dihydroisoquinolin-2(1H)- yl)-N-(1-(5-(4- (diethylphosphoryl)-3- (methylamino)phenyl)-3- (trifluoromethyl)-1H- pyrazol-1-yl)-2- methylpropan-2-yl)-3-(1- (difluoromethyl)-1H- pyrazol-4-yl)propanamide | 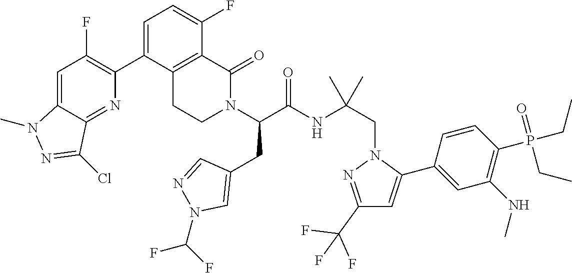 [SMILES: CCP(=O)(CC)c1ccc(-c2cc(C(F)(F)F)nn2CC(C)(C)NC(=O)[C@@H](Cc2cnn(C(F)F)c2)N2CCc3c(-c4nc5c(Cl)nn(C)c5cc4F)ccc(F)c3C2=O)cc1NC] | 935.3, 937.2 (M + H) |
| 129^{ad} | (R)-2-(5-(3-Chloro-6- fluoro-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(1-(5- (3-(dimethyl- phosphoryl)phenyl)- 3-(trifluoromethyl)- 1H-pyrazol-1-yl)-2- methylpropan-2- yl)propanamide | 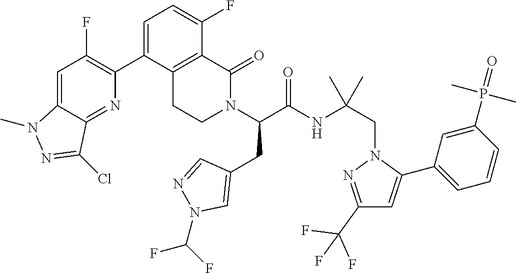 | 878.2, 880.1 (M + H) |
| 130^{ae} | (R)-2-(5-(3-Chloro-6- fluoro-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(1-(5- (dimethylphosphoryl)-3- (trifluoromethyl)-1H- pyrazol-1-yl)-2- methylpropan-2- yl)propanamide | 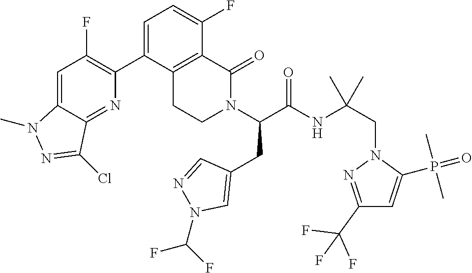 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3P(C)(C)=O)C4=O)c(F)cc21] | 802.1, 804.1 (M + H) |
| 131^{af} | (R)-2-(5-(3-Chloro-6- fluoro-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(2- methyl-1-(5-(methyl- sulfonamidomethyl)- 3-(trifluoromethyl)-1H- pyrazol-1-yl)propan-2- yl)propanamide | 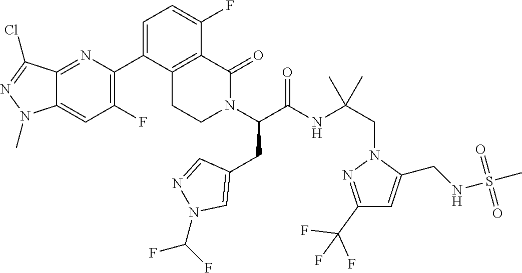 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3CNS(C)(=O)=O)C4=O)c(F)cc21] | 833.1, 835.1 (M + H) |
| 132^{ag} | (R)-N-(1-(3-Chloro-5-(2- methylpyrimidin-5-yl)- 1H-pyrazol-1-yl)-2- methylpropan-2-yl)-2-(5- (3-chloro-6-cyano-1- methyl-1H-pyrazolo[4,3- b]pyridin-5-yl)-8-fluoro-1- oxo-3,4- dihydroisoquinolin-2(1H)- yl)-3-(1-(difluoromethyl)- 1H-pyrazol-4- yl)propanamide | 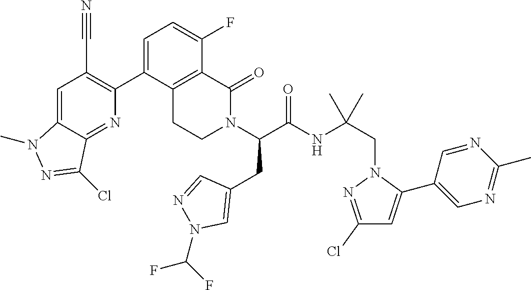 | 791.2, 793.1, 795.2 (M + H) |
| 133^{ah} | (R)-2-(5-(3-Chloro-6- cyano-1-methyl-1H- pyrazolo[4,3-b]pyridin-5- yl)-8-fluoro-1-oxo-3,4- dihydroisoquinolin-2(1H)- yl)-N-(1-(3-cyclopropyl-5- (2-methylpyrimidin-5-yl)- 1H-pyrazol-1-yl)-2- methylpropan-2-yl)-3-(1- (difluoromethyl)-1H- pyrazol-4-yl)propanamide | 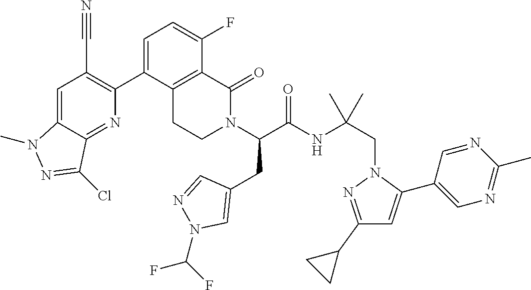 | 797.3, 799.3 (M + H) |
| 134^{ai} | (R)-2-(5-(3-Chloro-6- cyano-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(-1- hydroxy-2-methyl-1-(4- (trifluoromethyl)-1H- imidazol-2-yl)propan-2- yl)propenamide (Diastereomer 2) | 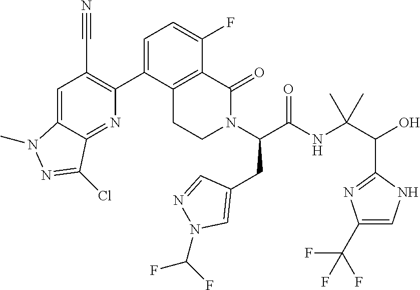 [SMILES: Cn1nc(Cl)c2nc(-c3ccc(F)c4c3CCN([C@H](Cc3cnn(C(F)F)c3)C(=O)NC(C)(C)C(O)c3nc(C(F)(F)F)c[nH]3)C4=O)c(C#N)cc21] | 749.2, 751.1 (M + H) |
| 135^{ae} | (R)-2-(5-(3-Chloro-6- cyano-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(2- methyl-1-(3-methyl-5-(2- methylpyrimidin-5-yl)- 1H-pyrazol-1-yl)propan-2- yl)propanamide | 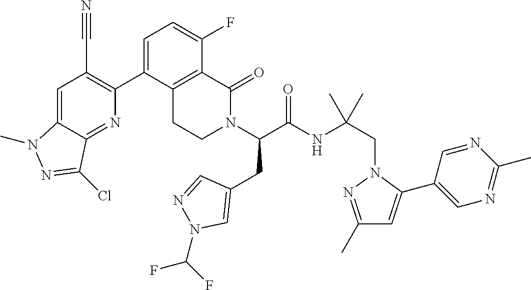 | 771.2, 773.1 (M + H) |
| 136^{aj} | (R)-2-(5-(3-Chloro-6- cyano-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(1-(3- fluoro-5-(2- methylpyrimidin-5-yl)- 1H-pyrazol-1-yl)-2- methylpropan-2- yl)propanamide | 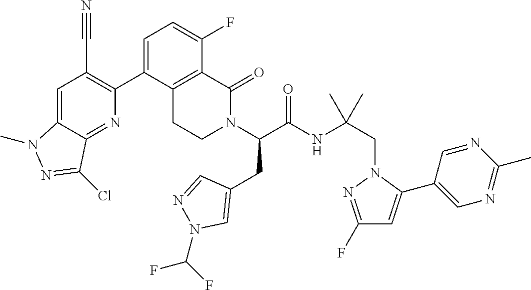 | 775.2, 777.1 (M + H) |
| 137^{ai} | (R)-2-(5-(3-Chloro-6- cyano-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(2- methyl-1-(5-(2- methylpyrimidin-5-yl)- 1H-pyrazol-1-yl)propan-2- yl)propanamide | 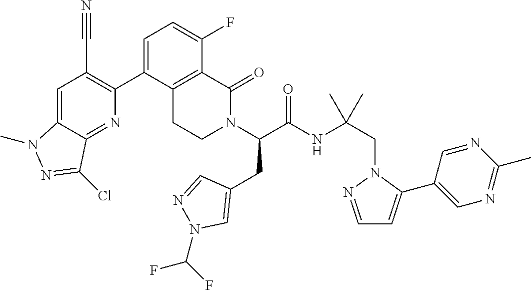 | 757.2, 759.1 (M + H) |
| 138^{ak} | (R)-2-(5-(3-Chloro-6- cyano-1-methyl-1H- pyrazolo[4,3-b]pyridin-5- yl)-8-fluoro-1-oxo-3,4- dihydroisoquinolin-2(1H)- yl)-3-(1-(difluoromethyl)- 1H-pyrazol-4-yl)-N-(2- methyl-1-(5-(-1- methylpyrrolidin-3-yl)-3- (trifluoromethyl)-1H- pyrazol-1-yl)propan-2- yl)propenamide (Diastereomer mixture) | 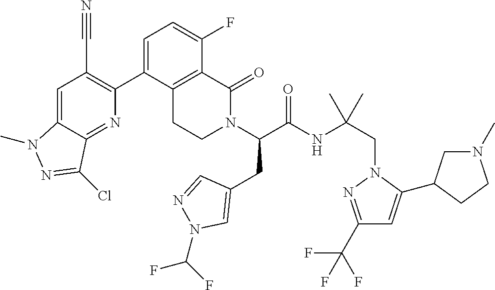 | 816.2, 818.3 (M + H) |
| 139^{al} | (R)-N-(1-(1H-indol-1-yl)- 2-methylpropan-2-yl)-2- (5-(3,6-difluoro-1-methyl- 1H-pyrazolo[4,3- b]pyridin-5-yl)-8-fluoro-1- oxo-3,4- dihydroisoquinolin-2(1H)- yl)-3-(1-(difluoromethyl)- 1H-pyrazol-4- yl)propanamide | 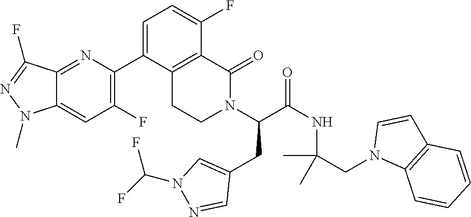 [SMILES: Cn1nc(F)c2nc(-c3ccc(F)c4c3CCN([C@H](Cc3cnn(C(F)F)c3)C(=O)NC(C)(C)Cn3ccc5ccccc53)C4=O)c(F)cc21] | 691.2 (M + H) |
| 140^{al} | (R)-2-(5-(3,6-Difluoro-1- methyl-1H-pyrazolo[4,3- b]pyridin-5-yl)-8-fluoro-1- oxo-3,4- dihydroisoquinolin-2(1H)- yl)-3-(1-(difluoromethyl)- 1H-pyrazol-4-yl)-N-(2- methyl-1-(6-methyl-1H- indol-1-yl)propan-2- yl)propanamide | 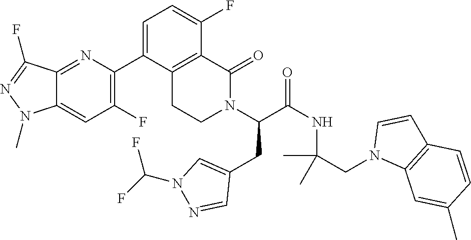 [SMILES: Cc1ccc2ccn(CC(C)(C)NC(=O)[C@@H](Cc3cnn(C(F)F)c3)N3CCc4c(-c5nc6c(F)nn(C)c6cc5F)ccc(F)c4C3=O)c2c1] | 705.2 (M + H) |
| 141^{m} | (R)-3-(1- (Difluoromethyl)-1H- pyrazol-4-yl)-2-(8-fluoro- 5-(6-fluoro-1-methyl-1H- pyrazolo[4,3-b]pyridin-5- yl)-1-oxo-3,4- dihydroisoquinolin-2(1H)- yl)-N-(2-methyl-1-(5-(1- methylpiperidin-4-yl)-3- (trifluoromethyl)-1H- pyrazol-1-yl)propan-2- yl)propanamide | 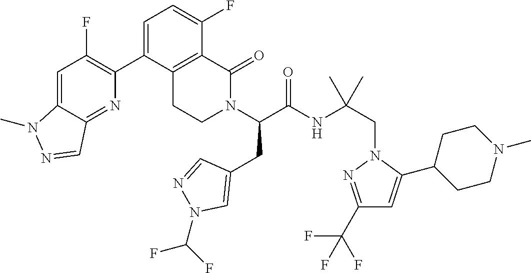 | 789.5 (M + H) |

^{a}Used HATU coupling reagent and DIEA base.
^{b}prep-HPLC (Column: F-Welch Xtimate C18 40 * 200 mm 7 μm; mobile phase: H₂O(0.225% FA)-ACN; B%: 26%-66%, 19 min)
^{c}purified by prep-HPLC (Column: F-Prepulite XP tC18 40 * 200 mm * 7 μm; mobile phase: water (10 mM NH₄HCO₃)-ACN; B%: 38%-78%, 20 min)
^{d}purified by prep-HPLC (column: F-Welch Xtimate C18 40 * 200 mm 7 μm; mobile phase: [A: H₂O(0.225% FA); B: ACN]; B%: 26.00%-66.00%, 19.00 min; flow rate: 60.00 ml/min). % e.e. was enriched from 78% to 99% by separated by SFC-1 (column: DAICEL CHIRALPAK AD(250 mm * 30 mm, 10 μm); mobile phase: [A: CO₂; B: IPA(0.1% NH₃H₂O)]; B%: 40.00%-40.00%, 4.00 min; flow rate: 90.00 ml/min)
^{e}purified by prep-HPLC (column: F-Welch Xtimate C18 40 * 200 mm 7 μm; mobile phase: [A: H₂O(0.225% FA); B: ACN]; B%: 24.00%-64.00%, 19.00 min; flow rate: 60.00 ml/min)
^{f}crude product was purified by flash silica gel chromatography (Biotage ®; 4 g Agela Silica Flash Column, Eluent of 0~4% MeOH/DCM gradient @ 30 mL/min) to give ~90 mg product (Rf = 0.4). ~90 mg product was further purified by prep-HPLC (column: F-Welch Xtimate C18 40 * 200 mm 7 μm; mobile phase: [A: H2O (0.225% FA); B: ACN]; B%: 28.00%-68.00%, 19.00 min; flow rate: 60.00 ml/min)
^{f}purified by prep-HPLC (column: F-Welch Xtimate C18 40 * 200 mm 7 μm; mobile phase: [A: H₂O(0.225% FA); B: ACN]; B%: 30.00%-70.00%, 19.00 min; flow rate: 60.00 ml/min)
^{h}purified by prep-HPLC (column: F-Welch Xtimate C18 40 * 200 mm 7 μm; mobile phase: [A: H₂O (10 mM NH₄HCO₃); B: ACN]; B%: 28.00%-58.00%, 19.00 min; flow rate: 30.00 ml/min)
ⁱCrude residue was purified by prep-HPLC (column: F-Welch Xtimate C18 40 * 200 mm 7 μm; mobile phase: [A: H₂O(0.225% FA); B: ACN]; B%: 22.00%-62.00%, 19.00 min; flow rate: 60.00 ml/min) then diastereomers were separated by SFC-1 (column: Daicel Chiralpak IBN (250 mm * 30 mm, 10 μm); mobile phase: [A: CO₂; B: EtOH(0.1% NH₃H₂O)]; B%: 45.00%-45.00%, 3.00 min; flow rate: 80.00 ml/min) and the second eluting isomer was collected.
^{j}purified by prep-HPLC (column: F-Welch Xtimate C18 40 * 200 mm 7 μm; mobile phase: [A: H₂O(0.225% FA); B: ACN]; B%: 40.00%-70.00%, 19.00 min; flow rate: 60.00 ml/min)
^{k}residue was purified by flash silica gel chromatography (Biotage ®; Agela ® Flash Column Silica-CS (4 g), Eluent of 5% MeOH/DCM @ 30 mL/min) and then by prep-HPLC (Column: YMC Triart C18 250 * 30 mm * 5 μm; mobile phase: H₂O(0.04% NH₃H₂O + 10 mM NH₄HCO₃)-ACN; B%: 50%-80%, 19 min)
^{l}Crude reaction mixture was poured into 10 mL of water to form a precipitate that was captured by filtration, washed 2 × with 5 mL of cold water to yield the desired product.
^{m}Purified by high pH reversed phase flash chromatography 50.0 g HP C18 GOLD column, eluted with a gradient of 5 to 70% ACN/(10 mM NH₄HCO₃ in water, with 5% MeOH) @ 60 mL/min.

### Example 142

### (R)—N-(1-(5-(2-(2-Aminopropan-2-yl)pyrimidin-5-yl)-3-(trifluoromethyl)-1H-pyrazol-1-yl)-2-methylpropan-2-yl)-2-(5-(3-chloro-6-fluoro-1-methyl-1H-pyrazolo[4,3-b]pyridin-5-yl)-8-fluoro-1-Oxo-3,4-dihydroisoquinolin-2(1H)-yl)-3-(1-(difluoromethyl)-1H-pyrazol-4-yl)propenamide, Formic Acid Salt

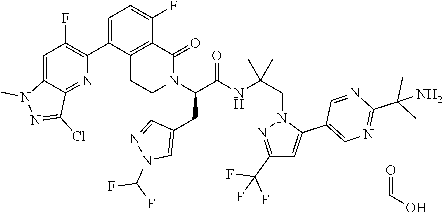 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)N)nc3)C4=O)c(F)cc21 ; O=CO]

To a solution of tert-butyl (R)-(2-(5-(1-(2-(2-(5-(3-chloro-6-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)carbamate (95 mg, 52 μmol, 53% purity) in 1,4-dioxane (1 mL) was added 2 M HCl in dioxane (1.0 mL, 2.0 mmol). The mixture was stirred at 20° C. for 16 h, concentrated under reduced pressure, and purified by preparative HPLC (C18, 40×200 mm, 7 μm, gradient 8-48% ACN in 0.225% aq. formic acid) to provide the title compound (15.99 mg, 35%) as white solid. ES-MS m/z 861.2 (M+H).

### Example 143

### (R)-3-(1-(2-(2-(5-(3,6-Difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)benzoic acid

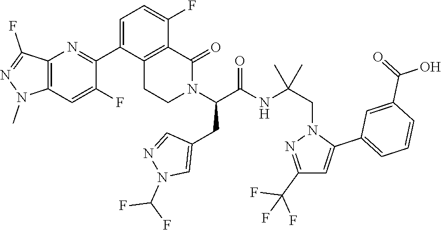 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3cccc(C(=O)O)c3)C4=O)c(F)cc21]

Methyl (R)-3-(1-(2-(2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)benzoate (71.8 mg, 76.6 umol, 90% purity) was dissolved in t-BuOH (1 mL) and treated with LiOH (2 M aq.) (350 μL, 700 μmol), stirred at RT for 100 min, then concentrated under vacuum. The residue was dissolved in MeOH, filtered, and purified by preparative reverse-phase HPLC (CSH C18, 30×250 mm, gradient 5-95% ACN in 10 mM aq. NH₄HCO₃₊₅% MeOH) to provide the title compound (44.6 mg, 69%) as a white solid. ES-MS m/z 830.4 (M+H).

### Example 144

### (R)-3-(1-(Difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)-N-(2-methyl-1-(5-pyrrolidin-2-yl)-3-(trifluoromethyl)-1H-pyrazol-1-yl)propan-2-yl)propenamide (Diastereomer 1)

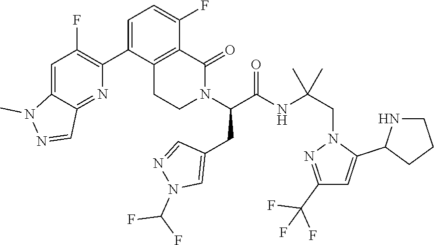 [SMILES: Cn1ncc2nc(-c3ccc(F)c4c3CCN([C@H](Cc3cnn(C(F)F)c3)C(=O)NC(C)(C)Cn3nc(C(F)(F)F)cc3C3CCCN3)C4=O)c(F)cc21]

The title compound was prepared by the procedure from Example 143 using tert-butyl (RS)-2-(1-(2-((R)-3-(1-(difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)propanamido)-2-methylpropyl)-3-(trifluoromethyl)-1H-pyrazol-5-yl)pyrrolidine-1-carboxylate (167.1 mg, 40% wt, 77.65 μmol) as the starting material. Diastereomers were separated by reverse phase HPLC (column: CSH (250 mm*30 mm); mobile phase: [A: NH₄OAc in H2O; B: ACN; B %: 40.00%-95.00%,40.00 min; flow rate: 40.00 ml/min) and the first eluting isomer was collected (11.5 mg, 19%). ES/MS m/z 761.6 (M+H).

### Example 145

### (R)—N-(1-(5-(-Azetidin-2-yl)-3-(trifluoromethyl)-1H-pyrazol-1-yl)-2-methylpropan-2-yl)-3-(1-(difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)propenamide (Diastereomer 1)

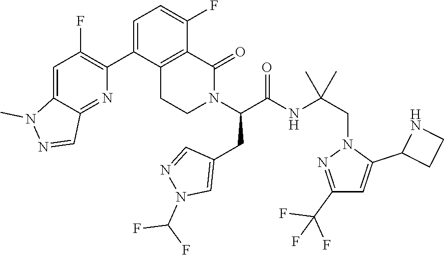 [SMILES: Cn1ncc2nc(-c3ccc(F)c4c3CCN([C@H](Cc3cnn(C(F)F)c3)C(=O)NC(C)(C)Cn3nc(C(F)(F)F)cc3C3CCN3)C4=O)c(F)cc21]

A solution of benzyl-2-(1-(2-((R)-3-(1-(difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)propanamido)-2-methylpropyl)-3-(trifluoromethyl)-1H-pyrazol-5-yl)azetidine-1-carboxylate (Diastereomer 1) (53.8 mg, 83% purity, 50.7 μmol) in EtOH (1.0 mL) was sparged with nitrogen for 5 min. Palladium on carbon (20% w/w loading, 50% wetted) (5.4 mg, 5.1 μmol) was added, and the resulting mixture was sparged with nitrogen for 5 min, then sparged with hydrogen for 5 min. The reaction mixture was stirred at RT under balloon pressure of hydrogen for 1.5 h. The mixture was filtered through a 0.45 μm syringe filter, rinsing with additional EtOH, and the filtrate was concentrated under a stream of nitrogen. Reversed phase purification of the residue (ACQUITY UPLC® CSH™ C-18 column, with a gradient of 5-95% ACN in water with 0.1% formic acid) provided the title compound (12.4 mg, 32%) as a white solid. ES/MS m/z 747.4 (M+H).

### Example 146

### (R)—N-(1-(5-(Aminomethyl)-3-(trifluoromethyl)-1H-pyrazol-1-yl)-2-methylpropan-2-yl)-3-(1-(difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)propenamide

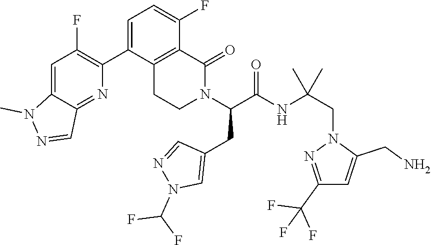 [SMILES: Cn1ncc2nc(-c3ccc(F)c4c3CCN([C@H](Cc3cnn(C(F)F)c3)C(=O)NC(C)(C)Cn3nc(C(F)(F)F)cc3CN)C4=O)c(F)cc21]

A solution of tert-butyl (R)-((1-(2-(3-(1-(difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)propanamido)-2-methylpropyl)-3-(trifluoromethyl)-1H-pyrazol-5-yl)methyl)carbamate (30.6 mg, 85% purity, 31.7 μmol) in MeOH (634 μL), was treated with HCl (4.0 M in 1,4-dioxane) (80 μL, 0.32 mmol). The mixture was stirred at RT for 4 h, then concentrated under a stream of nitrogen. Reversed phase purification of the residue (ACQUITY UPLC® CSH™ C-18 column, with a gradient of 5-95% ACN in 10 mM aqueous NH₄OAc) gave the title compound (14 mg, 60%) as a white solid. ES/MS m/z 721.4 (M+H).

### Example 147

### (R)-2-(5-(6-Cyano-1-methyl-2-oxo-1,2-dihydrothiazolo[5,4-b]pyridin-5-yl)-8-fluoro-1-oxo-3,4-dihydroisoquinolin-2(1H)-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propenamide

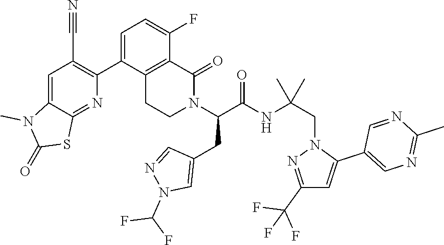

To a mixture of 5-chloro-1-methyl-2-oxo-1,2-dihydrothiazolo[5,4-b]pyridine-6-carbonitrile (17 mg, 92% Wt, 69 μmol), (R)-3-(1-(difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)propanamide (60 mg, 95% wt, 75 μmol) and Na₂CO₃ (26 mg, 0.25 mmol) in 1,4-dioxane (2 mL) and H₂O (0.2 mL) was added XPhos Pd G3 (15 mg, 18 μmol). The mixture was degassed 3× with N₂ and then the stirred mixture was heated to 90° C. After 2 h, the reaction was cooled to RT, diluted with EtOAc (10 mL), and washed with saturated aqueous NaCl (2×10 mL). The organic layer was then dried over Na₂SO₄, filtered, concentrated under reduced pressure, and purified by preparatory TLC on silica gel using 100% EtOAc as the mobile phase followed by reverse-phase HPLC (F-Welch Xtimate C18 40*200 mm 7 μm; using a gradient of 32%-72% ACN in 0.225% FA/water with a flow rate of 60 mL/min) to obtain the title compound (13.5 mg, 23%) as a white solid. ES-MS m/z 824.2 (M+H).

### Example 148

### (R)-2-(5-(6-cyano-1-methyl-2-oxo-1,2-dihydrothiazolo[5,4-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

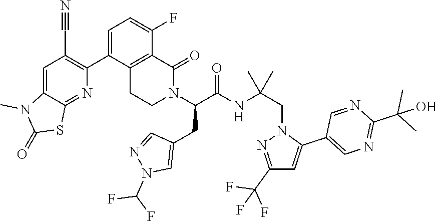 [SMILES: Cn1c(=O)sc2nc(-c3ccc(F)c4c3CCN([C@H](Cc3cnn(C(F)F)c3)C(=O)NC(C)(C)Cn3nc(C(F)(F)F)cc3-c3cnc(C(C)(C)O)nc3)C4=O)c(C#N)cc21]

To a mixture of (R)-3-(1-(difluoromethyl)-1H-pyrazol-4-yl)-2-(8-fluoro-1-oxo-5-(4,4,5,5-tetramethyl-1,3,2-dioxaborolan-2-yl)-3,4-dihydroisoquinolin-2 (1H)-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide (0.110 g, 71% Wt, 97.1 μmol), 5-chloro-1-methyl-2-oxo-1,2-dihydrothiazolo[5,4-b]pyridine-6-carbonitrile (20 mg, 88 μmol) and Na₂CO₃ (21 mg, 0.20 mmol) in 1,4-dioxane (1 mL) and H₂O (0.1 mL) was added XPhos Pd G3 (16 mg, 19 μmol). The mixture was degassed with N₂ (3×), then stirring was initiated and the mixture was heated to 90° C. under an atmosphere of N₂. After 1 h, the reaction was removed from heating, filtered, and the filter cake was washed with ethyl acetate (3×8 mL). The filtrate was concentrated under reduced pressure and purified by silica gel chromatography using a mobile phase of 0-3% MeOH in DCM followed by prep-SFC purification [stationary phase-DAICEL CHIRALPAK AD (250 mm×30 mm, 10 μm); mobile phase: 40% EtOH (0.1% aq. ammonia) in supercritical CO₂ at a flow rate of 80.00 ml/min] to afford the title compound (23.4 mg, 30% yield) as a white solid. ES-MS m/z 868.2 (M+H).

### Example 149

### (R)-2-(5-(6-Cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

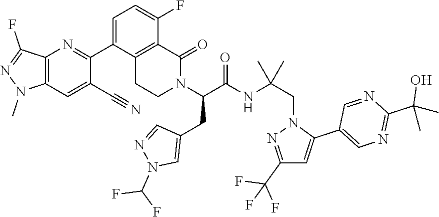 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)O)nc3)C4=O)c(C#N)cc21]

A mixture of 2-(5-(1-(2-amino-2-methylpropyl)-3-(trifluoromethyl)-1H-pyrazol-5-yl)pyrimidin-2-yl)propan-2-ol hydrochloride (1.7 g, 3.6 mmol), (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (1.7 g, 2.9 mmol) and HATU (1.5 g, 3.9 mmol) in DMA (20 mL) was cooled to 0° C. and then DIEA (2.5 mL, 15 mmol) was added. The reaction was allowed to continue for 30 min, diluted with H₂O (100 mL) and extracted with EtOAc (3×100 mL). The combined organic layers were washed with sat. aq. NaCl (2×100 mL), dried over anhydrous Na₂SO₄, filtered, concentrated under reduced pressure, and purified by silica gel chromatography using a gradient of 0-4% DCM in MeOH followed by chiral prep-SFC purification [column-REGIS (S,S) WHELK-01 (250 mm×30 mm, 10 μm); mobile phase-40% EtOH (0.1% aq. NH₃) in supercritical CO₂ at a flow rate of 140 mL/min] to afford the title compound (1446.7 mg, 58% yield) as a yellow solid. ES-MS m/z 853.3 (M+H).

### Preparation 518

### (R)-Diallyl (2-(5-(1-(2-(2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)phosphate

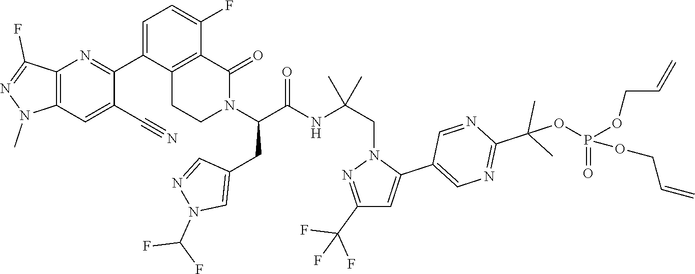 [SMILES: C=CCOP(=O)(OCC=C)OC(C)(C)c1ncc(-c2cc(C(F)(F)F)nn2CC(C)(C)NC(=O)[C@@H](Cc2cnn(C(F)F)c2)N2CCc3c(-c4nc5c(F)nn(C)c5cc4C#N)ccc(F)c3C2=O)cn1]

To (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide (458.9 mg, 538.1 μmol) was added DCM (11 mL) and 1H-tetrazole (0.45 M in ACN) (2.6 mL, 1.2 mmol). Stirring was initiated at RT and then diallyl n,n-diisopropylphosphoramidite (300 μL, 1.13 mmol) was added. After 4 h, the reaction was cooled to 0° C. and hydrogen peroxide (800 μL, 30% Wt, 7.73 mmol) was added. After stirring for 15 min, the mixture was diluted with EtOAc and sat. aq. sodium thiosulfate. The organic layer was removed, and the aqueous layer was extracted with additional EtOAc (2×). The combined organic layers were then washed with sat. aq. NH₄Cl, sat. aq. NaHCO₃. The mixture was dried over Na₂SO₄, filtered, and concentrated to provide a residue. The residue was purified by silica gel chromatography (0-25% EtOAc/heptane) to afford the title compound (506 mg, 91% yield) as a white solid. ES/MS m/z 1013.5 (M+H).

### Example 150

### Ammonium (R)-2-(5-(1-(2-(2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl hydrogen phosphate

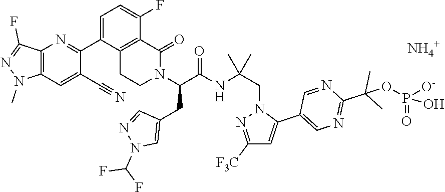 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)OP(=O)([O-])O)nc3)C4=O)c(C#N)cc21 ; [NH4+]]

(R)-Diallyl (2-(5-(1-(2-(2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)phosphate (505.8 mg, 499.4 μmol), tetrakis(triphenylphosphine)palladium(0) (29.8 mg, 25.8 μmol), and a magnetic stir-bar were added to a scintillation vial and placed under an inert atmosphere by placing under reduced pressure and then backfilling with argon 3×. The vial was then cooled to 0° C., and THF (5.0 mL) was added, followed immediately by phenylsilane (180 μL, 1.45 mmol). Stirring was initiated, and the reaction was allowed to proceed for 20 min, at which point it was halted by dilution with MeOH (5.0 mL) and by the addition of ˜50 mg of SiliaMetS® Thiol Scavenger. The resulting mixture was stirred vigorously for 20 min and then filtered through a diatomaceous earth plug. The filtrate was concentrated under reduced pressure, re-suspended in MeOH (an insoluble precipitate had passed through the diatomaceous earth pad) and then an additional ˜75 mg of SiliaMetS® Thiol Scavenger was added. The resulting suspension was stirred for 1.5 h, after which the mixture was filtered through a second diatomaceous earth plug, concentrated under reduced pressure, and purified by reverse phase prep-HPLC [stationary phase: CSH 30 mm×250 mm; mobile phase: gradient of 40-95% ACN in water (with 0.1% formic acid)]. The purified material was then dissolved in MeOH, treated with ammonium acetate (35 mg, 0.45 mmol) to adjust the salt form, concentrated under a stream of nitrogen, and lyophilized from a mixture of water and acetonitrile to afford the title compound (316.6 mg, 64% yield) as a white solid. ES-MS m/z 933.4 (M+H).

### Example 151

### (R)-2-(5-(6-Cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(6-(2-hydroxypropan-2-yl)pyridin-3-yl)-3-(trifluoromethyl)-1H-pyrazol-1-yl)-2-methylpropan-2-yl)propanamide

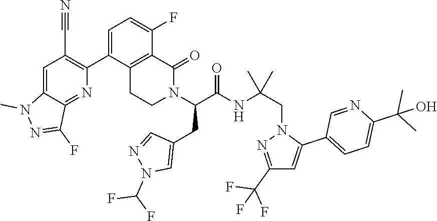 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3ccc(C(C)(C)O)nc3)C4=O)c(C#N)cc21]

To a mixture of 2-(5-(1-(2-amino-2-methylpropyl)-3-(trifluoromethyl)-1H-pyrazol-5-yl)pyridin-2-yl)propan-2-ol trifluoromethanesulfonate (100 mg, 74% Wt, 150 μmol) and (R)-2-(5-(6-cyano-3-fluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (88 mg, 0.16 mmol) in DMA (1 mL) was added diethyl (4-oxo-1,2,3-benzotriazin-3-yl)phosphate (100 mg, 331 μmol) and 4-methylmorpholine (90 μL, 0.81 mmol). The resulting mixture was stirred at 20° C. for 16 h, after which it was diluted with H₂O (8 mL) and extracted with EtOAc (3×8 mL). The combined organic layers were dried over anhydrous Na₂SO₄, filtered, concentrated under reduced pressure, and purified by prep-HPLC [column-YMC Triart C18 250×30 mm, 5 μm; mobile phase-gradient of 48-78% ACN in H₂O (10 mM NH₄HCO₃) at a flow rate of 25 mL/min] followed by chiral prep-SFC purification (column—(S,S) WHELK-01 (250 mm*30 mm, 5 μm); mobile phase-40% EtOH (0.1% aq. NH₃) in supercritical CO₂ at a flow rate of 80 mL/min) to afford the title compound (27.59 mg, 21% yield) as a white solid. ES-MS m/z 852.3 (M+H).

### Example 152

### (R)-2-(5-(3,6-Difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

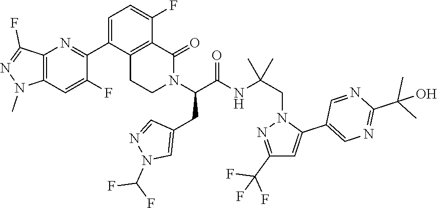 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)O)nc3)C4=O)c(F)cc21]

(R)-2-(5-(3,6-Difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (27.9 mg, 53.6 μmol), HATU (24.4 mg, 64.2 μmol) and a magnetic stir-bar were added to the scintillation vial containing 2-(5-(1-(2-amino-2-methylpropyl)-3-(trifluoromethyl)-1H-pyrazol-5-yl)pyrimidin-2-yl)propan-2-ol (41.6 mg, 73% purity, 88.4 μmol). The solids were dissolved in DMF (1 mL) and then TEA (10.9 mg, 15.0 μL, 2.01 Eq, 108 μmol) was added. The reaction was stirred at RT for 3 days, then slowly added to 20 mL H₂O, stirred for 1 h, centrifuged at 3000 rpm for 15 min, filtered, and the collected solids were eluted with MeOH. The methanolic filtrate was concentrated under N₂ and purified by silica gel chromatography (gradient 0-100% 3:1 EtOH:EtOAc in heptane) to provide the title compound (43.5 mg, 94%) as a clear glassy solid. ES-MS m/z 846.4 (M+H).

### Example 153

### (R)-2-(5-(3-chloro-6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

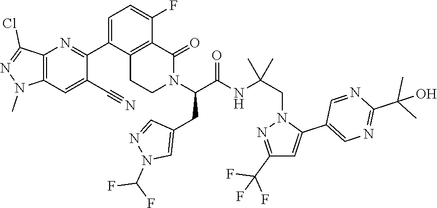 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)O)nc3)C4=O)c(C#N)cc21]

2-(5-(1-(2-amino-2-methylpropyl)-3-(trifluoromethyl)-1H-pyrazol-5-yl)pyrimidin-2-yl)propan-2-ol (592 mg, 1.73 mmol) was dissolved in DMA (11.5 mL) and added to a 40 mL scintillation vial containing (R)-2-(5-(3-chloro-6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (783 mg, 1.38 mmol). The reaction mixture was stirred on an ice bath, then 4-methylmorpholine (0.46 mL, 4.2 mmol) was added followed by DEPBT (598 mg, 2.00 mmol). The reaction mixture was stirred at RT overnight, diluted with sat. aq. NaCl and EtOAc, and the aqueous layer was removed. The organic layer was washed with sat. aq. NaCl (3×), 1 M aq. HCl (1×), sat aq. Na₂CO₃ (1×), and sat. aq. NaCl (1×), dried over Na₂SO₄, filtered, concentrated, and chromatographed (Whelk-O SS 30×250 mm, 5 μm, 85 mL/min, isocratic 40% MeOH in CO₂, 40° C.) to provide the title compound (693 mg, 57%) as a rose-cream colored solid.

### Preparation 520

### (R)-Diallyl (2-(5-(1-(2-(2-(5-(3-chloro-6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)phosphate

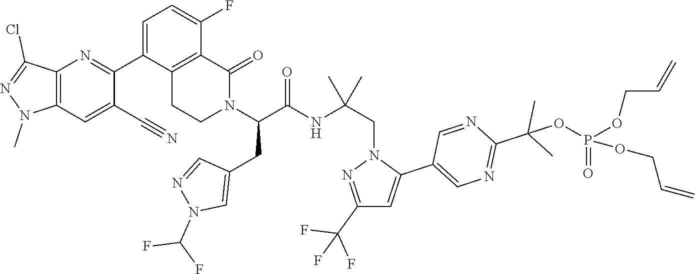 [SMILES: C=CCOP(=O)(OCC=C)OC(C)(C)c1ncc(-c2cc(C(F)(F)F)nn2CC(C)(C)NC(=O)[C@@H](Cc2cnn(C(F)F)c2)N2CCc3c(-c4nc5c(Cl)nn(C)c5cc4C#N)ccc(F)c3C2=O)cn1]

To a solution of (R)-2-(5-(3-chloro-6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide (176.4 mg, 202.9 μmol) in DCM (2.0 mL) was added 1H-tetrazole (0.45 M solution in ACN) (700 μL, 315 μmol) followed by diallyl-n,n-diisopropylphosphoramidite (70 μL, 0.26 mmol). The reaction was allowed to stir overnight. Then, the reaction was cooled to 0° C. and hydrogen peroxide (320 μL, 30% Wt, 3.09 mmol) was added. After stirring at 0° C. for 1.5 h, the reaction was diluted with EtAOc (50 mL) and sat. aq. sodium thiosulfate (50 mL). The layers were separated and the aqueous layer was extracted with EtOAc (2×50 mL). The combined organic layer was washed with sat. aq. NH₄Cl (40 mL), sat. aq. NaHCO₃ (40 mL) and sat. aq. NaCl (40 mL), dried over anhydrous Na₂SO₄, filtered and concentrated. The residue was purified by silica gel chromatography (0-100% [3:1 EtOAc/EtOH]/heptane) to afford the title compound (207.1 mg, 57% yield, 57% purity) as a white solid. ES-MS m/z 1027.2 (M−H)

### Example 154

### Ammonium (R)-2-(5-(1-(2-(2-(5-(3-chloro-6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl hydrogen phosphate

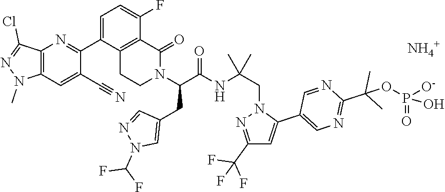 [SMILES: Cn1nc(Cl)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)OP(=O)([O-])O)nc3)C4=O)c(C#N)cc21 ; [NH4+]]

A solution of (R)-diallyl (2-(5-(1-(2-(2-(5-(3-chloro-6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)phosphate (207.1 mg, 57% purity, 114.7 μmol) in THF (2.3 mL) was sparged with argon for ˜5 min. Tetrakis (triphenylphosphine)palladium(0) (7.7 mg, 6.7 μmol) was added followed immediately by phenylsilane (52 mg, 60 μL, 4.2 Eq, 0.48 mmol). After stirring at ambient temperature under an atmosphere of argon for ˜2 h, the reaction mixture was diluted with MeOH (˜2.5 mL) and treated with SiliaMetS® Thiol Metal Scavenger (˜20 mg). The resulting mixture was allowed to stir for ˜45 min, and then it was filtered through a pad of diatomaceous earth, using EtOAc (˜50 mL) to aid transfer and to rinse the filter cake. The filtrate was concentrated, and the residue was purified by reversed phase prep-HPLC (eluting with a gradient of 5-95% CH₃CN in 10 mM aqueous NH₄OAc) to afford the title compound (56.4 mg, 48% yield) as a white solid. ES/MS m/z 949.2 (M+H).

### Preparation 519

(R)-diallyl (2-(5-(1-(2-(2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)phosphate

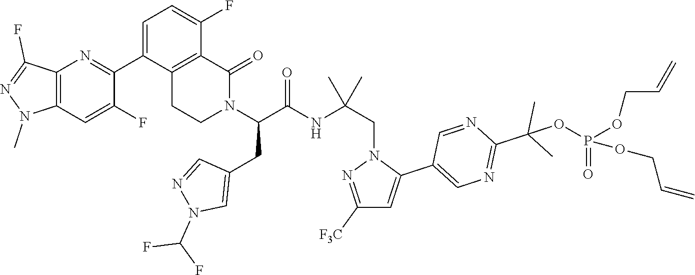 [SMILES: C=CCOP(=O)(OCC=C)OC(C)(C)c1ncc(-c2cc(C(F)(F)F)nn2CC(C)(C)NC(=O)[C@@H](Cc2cnn(C(F)F)c2)N2CCc3c(-c4nc5c(F)nn(C)c5cc4F)ccc(F)c3C2=O)cn1]

Prepared as described in Preparation 518 using (R)-2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide. ES-MS m/z 1006.4 (M+H).

### Example 155

### Ammonium (R)-2-(5-(1-(2-(2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl dihydrogen phosphate

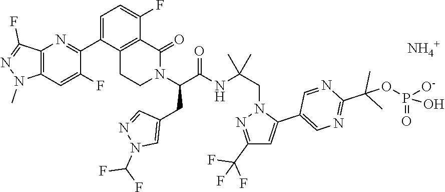 [SMILES: Cn1nc(F)c2nc(-c3ccc(F)c4c3CCN([C@H](Cc3cnn(C(F)F)c3)C(=O)NC(C)(C)Cn3nc(C(F)(F)F)cc3-c3cnc(C(C)(C)OP(=O)([O-])O)nc3)C4=O)c(F)cc21 ; [NH4+]]

The title compound was prepared in similar manner as described in Example 154 using (R)-diallyl (2-(5-(1-(2-(2-(5-(3,6-difluoro-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)phosphate. ES-MS m/z 926.4 (M+H).

### Example 156

### (R)-2-(5-(6-Cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(1-(5-(6-(2-hydroxypropan-2-yl)pyridin-3-yl)-3-(trifluoromethyl)-1H-pyrazol-1-yl)-2-methylpropan-2-yl)propanamide

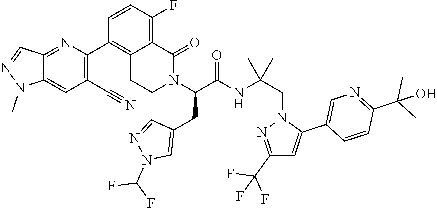 [SMILES: Cn1ncc2nc(-c3ccc(F)c4c3CCN([C@H](Cc3cnn(C(F)F)c3)C(=O)NC(C)(C)Cn3nc(C(F)(F)F)cc3-c3ccc(C(C)(C)O)nc3)C4=O)c(C#N)cc21]

To a solution of 2-(5-(1-(2-amino-2-methylpropyl)-3-(trifluoromethyl)-1H-pyrazol-5-yl)pyridin-2-yl)propan-2-ol dihydrochloride (51 mg, 0.12 mmol), (R)-2-(5-(6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid (64 mg, 0.12 mmol) in DMA (2 mL) were added DIEA (100 μL, 574 μmol) and HATU (73 mg, 0.19 mmol). The mixture was stirred at 0° C. for 0.5 h. The mixture was diluted with EtOAc (30 mL) and washed with water (30 mL×3). The organic layer was dried over anhydrous Na₂SO₄, filtered and concentrated under reduced pressure. The residue was purified by prep-HPLC (column: F-Welch Xtimate C18 40×200 mm 7 μm; mobile phase: [A: H₂O (0.05% NH₃H₂O+10 mM NH₄HCO₃); B: ACN]; B %: 28%-68%; flow rate: 60.00 mL/min) to give the title compound (40.26 mg, 40%) as a white solid. ES-MS m/z 834.3 (M+H); e.e.=84.68%.

### Example 157

### (R)-3-(1-(Difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)-N-(1-(5-(2-(hydroxymethyl)pyrimidin-5-yl)-3-(trifluoromethyl)-1H-pyrazol-1-yl)-2-methylpropan-2-yl)propanamide

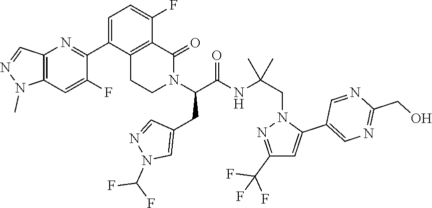 [SMILES: Cn1ncc2nc(-c3ccc(F)c4c3CCN([C@H](Cc3cnn(C(F)F)c3)C(=O)NC(C)(C)Cn3nc(C(F)(F)F)cc3-c3cnc(CO)nc3)C4=O)c(F)cc21]

A mixture of 1-(5-(2-(hydroxymethyl)pyrimidin-5-yl)-3-(trifluoromethyl)-1H-pyrazol-1-yl)-2-methylpropan-2-aminium chloride (21 mg, 60 μmol), (R)-3-(1-(difluoromethyl)-1H-pyrazol-4-yl)-2-(8-fluoro-5-(6-fluoro-1-methyl-1H-pyrazolo[4,3-b]pyridin-5-yl)-1-oxo-3,4-dihydroisoquinolin-2 (1H)-yl)propanoic acid (20.5 mg, 40.8 μmol) and DMA (0.5 mL) was cooled in an ice bath then 4-methylmorpholine (25 μL, 0.23 mmol) was added followed by diethyl (4-oxobenzo[d][1,2,3]triazin-3 (4H)-yl)phosphate (26.5 mg, 88.6 μmol). The reaction was allowed to continue overnight, gradually warming to RT as the ice-bath melted overnight. Diluted with MeOH, filtered through a syringe filter and purified by revers phase chromatography (column: UPLC CSH C18, 2.1 mm×50 mm, 1.7 μm, Mobile phase: A-water, B-ACN, Mobile phase additive: 0.1% Formic Acid, Flow rate: 0.8 mL/min, gradient: 5 to 95% B) to give the title compound (13.4 mg, 40% yield) as a white solid. ES-MS m/z 800.4 (M+H).

### Example 158

### (R)-2-(5-(3-Chloro-6-cyano-1-methyl-1H-pyrazolo[4,3-b]pyridin-5-yl)-8-fluoro-1-oxo-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)-3-(1-(methyl-d3)-1H-pyrazol-4-yl)propanamide

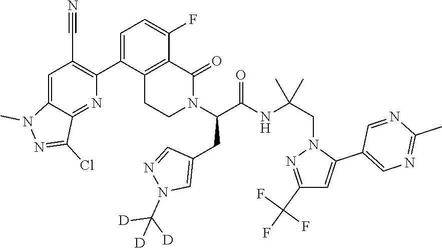

A mixture of (R)-2-(8-fluoro-1-oxo-5-(4,4,5,5-tetramethyl-1,3,2-dioxaborolan-2-yl)-3,4-dihydroisoquinolin-2 (1H)-yl)-N-(2-methyl-1-(5-(2-methylpyrimidin-5-yl)-3-(trifluoromethyl)-1H-pyrazol-1-yl)propan-2-yl)-3-(1-(methyl-d3)-1H-pyrazol-4-yl)propanamide (2.5 g, 3.1 mmol), 3,5-dichloro-1-methyl-1H-pyrazolo[4,3-b]pyridine-6-carbonitrile (0.90 g, 3.9 mmol), sodium carbonate (1.7 g, 16 mmol) and 1,1′-bis(di-t-butylphosphino)ferrocene palladium dichloride (0.500 g, 767 μmol) in 1,4-dioxane (80 mL) and water (8 mL) was degassed and purged with N₂ 3 times and stirred at 80° C. for 3 h. The mixture was filtered and concentrated under reduced pressure and the residue was purified by silica gel chromatography using a gradient of 0 to 5% MeOH in DCM, then re-purified by prep-HPLC (column: Phenomenex luna C18 250*50 mm, 10 μm; mobile phase: [A: H₂O (10 mM NH₄HCO₃); B: ACN]; B %: 25%-65%; flow rate: 100.00 mL/min) to give the title compound (1492 mg, 60% yield) as a white solid. ES-MS m/z 792.2 (M+H), e.e. =97.7%.

### Example 159

### (R)-2-(8-Fluoro-5-(6-fluoro-1-methyl-1H-pyrazolo[4,3-b]pyridin-5-yl)-1-oxo-3,4-dihydroisoquinolin-2 (1H)-yl)-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide

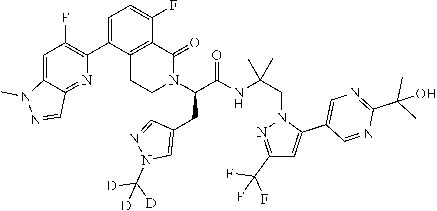 [SMILES: [2H]C([2H])([2H])n1cc(C[C@H](C(=O)NC(C)(C)Cn2nc(C(F)(F)F)cc2-c2cnc(C(C)(C)O)nc2)N2CCc3c(-c4nc5cnn(C)c5cc4F)ccc(F)c3C2=O)cn1]

A 20-mL vial was charged with (R)-2-(5-bromo-8-fluoro-1-oxo-3,4-dihydroisoquinolin-2 (1H)-yl)-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide (300 mg, 414 μmol), Pd(dppf)Cl₂·CH₂Cl₂ (34 mg, 42 μmol), bis(pinacolato)diborane (263 mg, 1.04 mmol), and potassium acetate (163 mg, 1.66 mmol). The vial was purged with nitrogen via a needle through the septum, then 1,4-dioxane (4.14 mL) was added. The resulting mixture was sparged with nitrogen for 10 min, then the needle was lifted into the headspace of the vial. The reaction was heated to 80° C. for 2.5 h. To the freshly prepared solution of (R)-2-(8-fluoro-1-oxo-5-(4,4,5,5-tetramethyl-1,3,2-dioxaborolan-2-yl)-3,4-dihydroisoquinolin-2 (1H)-yl)-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide (320 mg, 415 μmol) in 1,4-dioxane (4.15 mL) at RT was added 5-chloro-6-fluoro-1-methyl-1H-pyrazolo[4,3-b]pyridine (115 mg, 620 μmol), XPhos Pd G4 (36 mg, 42 μmol), and a solution of potassium phosphate (264 mg, 1.24 mmol) in water (747 μL). The resulting mixture was sparged with nitrogen for 10 min, then the sparge needle was lifted into the headspace of the vial. The reaction mixture was heated to 90° C. for 6 h under a positive pressure of nitrogen. The reaction mixture was stirred with SilaMetS thiol scavenger (˜50 mg), then was filtered through a 0.45 μm syringe filter, rinsing with MeOH. The filtrate was concentrated under a stream of nitrogen. The residue was purified by revers phase chromatography (column: UPLC CSH C18, 2.1 mm×50 mm, 1.7 μm; Mobile phase: A: Water B: Acetonitrile; Mobile phase additive: 0.1% Formic Acid; Flow rate: 0.8 mL/min; gradient: 5 to 95% B) to give the title compound (137 mg, 41.6% yield) as a white powder. ES-MS m/z 795.4 (M+H)

### Example 160

### (2R)-2-(5-(6-Cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)-N-(2-(5-methyl-6-oxo-2-(trifluoromethyl)-4,5,6,7-tetrahydropyrazolo[1,5-a]pyrazin-7-yl)propan-2-yl)propanamide (Isomer 1)

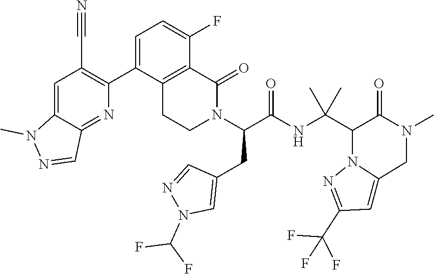

The title compound was prepared in similar manner as described in Example 157 from 7-(2-aminopropan-2-yl)-5-methyl-2-(trifluoromethyl)-4,5-dihydropyrazolo[1,5-a]pyrazin-6 (7H)-one (Isomer 1) and (R)-2-(5-(6-cyano-1-methyl-1H-pyrazolo[4,3-b]pyridin-5-yl)-8-fluoro-1-oxo-3,4-dihydroisoquinolin-2(1H)-yl)-3-(1-(difluoromethyl)-1H-pyrazol-4-yl)propanoic acid. ES-MS m/z 768.1 (M+H)

Functional hGIP-R Assay

Functional activity was determined using cAMP formation in HEK-293 clonal cell lines expressing hGIP-R. hGIP-R receptor expressing cells were treated with compound (20 point concentration-response curve in DMSO, 2-fold Labcyte Echo direct dilution, 384 well plate Corning Cat #3570) in DMEM (Gibco Cat #31053) supplemented with 1× GlutaMAX™ (Gibco Cat #35050), 0.1% bovine casein (Sigma C4765-10ML), 250 μM IBMX (3-Isobutyl-1-methylxanthine, Acros Cat #228420010) and 20 mM HEPES (Gibco Cat #15630) in a 20 μL assay volume (final DMSO concentration was 1%). After a 30 min incubation at 37° C., the resulting increase in intracellular cAMP was quantitatively determined using the Revvity CAMP Dynamic 2 HTRF Assay Kit (62AM4PEJ). Briefly, cAMP levels within the cell were detected by adding the cAMP-d2 conjugate in cell lysis buffer (10 μL) followed by the antibody anti-cAMP-Eu³⁺-Cryptate, also in cell lysis buffer (10 μL). The resulting competitive assay was incubated for at least 60 min at RT, then detected using a BMG Labtech PHERAstar FSX instrument with excitation at 320 nm and emission at 665 nm and 620 nm. PHERAstar units (emission at 665 nm/620 nm*10,000) are inversely proportional to the amount of cAMP present and were converted to nM cAMP per well using a cAMP standard curve. The amount of cAMP generated (nM) in each well was converted to a percent of the maximal response observed with human GIP (1-42) OH. A relative EC₅₀ value and percent top (Eₘₐₓ) were derived by non-linear regression analysis using the percent maximal response vs. the concentration of compound added, fitted to a four-parameter logistic equation.

Results. The Examples below were tested using the above-described assay and found to have an EC₅₀ of 100 nM or less. This data indicates that these compounds are agonists of the human GIPR. Functional data for particular exemplified compounds is in Tables 56, 57, and 58 below.

TABLE 56
Functional cAMP Potency (EC₅₀) and Efficacy (Eₘₐₓ) for compounds
incubated at 37° C. in the presence of 0.1% bovine casein.

| | |
|---|---|
| Example | Human GIP-R |

| No. | EC₅₀, nM (SEM, n)^{a} | Eₘₐₓ, %^{b} |
|---|---|---|
| 1 | 3.66 (0.148, n = 4) | 99.8 ± 8.50 |
| 2 | 5.48 (2.09, n = 2) | 72.3 ± 0.179 |
| 3 | 16.0 (1.12, n = 2) | 76.6 ± 3.50 |
| 4 | 15.6 (2.20, n = 2) | 127 ± 4.16 |
| 5 | 2.74 (0.126, n = 3) | 93.4 ± 7.03 |
| 6 | 2.03 (0.501, n = 2) | 103 ± 2.93 |
| 7 | 5.36 (0.868, n = 2) | 84.5 ± 0.447 |
| 8 | 51.1 (4.11, n = 2) | 58.3 ± 0.0344 |
| 9 | 32.9 (10.1, n = 2) | 107 ± 0.961 |
| 10 | 5.09 (0.652, n = 2) | 98.4 ± 5.83 |
| 11 | 3.70 (0.150, n = 2) | 92.9 ± 5.49 |
| 12 | 3.85 (0.579, n = 2) | 91.0 ± 6.42 |
| 13 | 65.3 (48.5, n = 2) | 55.3 ± 0.536 |
| 14 | 1.46 (0.164, n = 2) | 107 ± 2.57 |
| 15 | 5.52 (0.00508, n = 2) | 112 ± 3.96 |
| 16 | 31.7 (1.88, n = 2) | 101 ± 0.956 |
| 17 | 1.95 (0.233, n = 2) | 90.3 ± 6.56 |
| 18 | 2.26 (0.730, n = 2) | 104 ± 1.07 |
| 19 | 2.08 (0.0902, n = 2) | 87.9 ± 0.111 |
| 20 | 55.8 (2.21, n = 2) | 30.7 ± 4.39 |
| 21 | 68.0 (2.99, n = 2) | 78.8 ± 6.09 |
| 22 | 1.10 (0.189, n = 2) | 29.5 ± 3.62 |
| 23 | 15.9 (2.48, n = 2) | 72.6 ± 2.63 |
| 24 | 6.64 (1.60, n = 2) | 87.6 ± 1.14 |
| 25 | 0.412 (0.0680, n = 3) | 25.9 ± 2.60 |
| 26 | 0.692 (0.146, n = 6) | 36.7 ± 3.13 |
| 27 | 1.05 (0.261, n = 2) | 37.2 ± 2.25 |
| 28 | 2.13 (0.467, n = 2) | 28.4 ± 1.92 |
| 29 | 3.55 (0.693, n = 3) | 19.4 ± 3.36 |
| 30 | 47.3 (8.20, n = 2) | 83.9 ± 0.203 |
| 31 | 43.7 (2.73, n = 2) | 79.3 ± 3.92 |
| 32 | 15.6 (1.87, n = 2) | 20.5 ± 0.870 |
| 33 | 45.5 (16.0, n = 2) | 81.8 ± 2.00 |
| 34 | 86.7 (2.86, n = 2) | 89.5 ± 12.9 |
| 35 | 82.7 (22.3, n = 2) | 33.3 ± 4.40 |
| 36 | 28.3 (7.90, n = 2) | 77.1 ± 0.367 |
| 37 | 3.72 (0.971, n = 2) | 76.0 ± 2.57 |
| 38 | 20.8 (2.24, n = 2) | 96.9 ± 0.757 |
| 39 | 51.7 (8.70, n = 2) | 106 ± 8.44 |
| 40 | 6.93 (1.88, n = 3) | 38.5 ± 6.06 |
| 41 | 7.71 (0.293, n = 2) | 51.2 ± 0.273 |
| 42 | 4.81 (0.412, n = 2) | 29.7 ± 1.14 |
| 43 | 35.9 (2.07, n = 3) | 106 ± 4.94 |
| 44 | 33.1 (1.28, n = 2) | 78.1 ± 3.26 |
| 45 | 0.838 (0.00476, n = 2) | 98.4 (3.59, n = 2) |
| 46 | 0.321 | 116 |
| 47 | 0.596 (0.0846, n = 3) | 105 (4.65, n = 3) |
| 48 | 1.08 (0.0984, n = 5) | 109 (3.00, n = 5) |
| 49 | 12.9 (0.623, n = 2) | 66.7 (2.32, n = 2) |
| 50 | 11.7 (0.109, n = 2) | 94.2 (3.69, n = 2) |
| 51 | 6.29 (0.392, n = 2) | 106 (0.353, n = 2) |
| 52 | 3.11 (0.141, n = 2) | 97.1 (3.45, n = 2) |
| 53 | 1.75 (0.312, n = 2) | 96.7 (2.83, n = 2) |
| 54 | 13.6 (1.00, n = 2) | 95.2 (1.09, n = 2) |

^{a}EC₅₀, nM = the Geometric Mean with the Standard Error of the Mean followed by the number of observations in parenthesis.
^{b}Eₘₐₓ, % = the Arithmetic Mean ± the Standard Error of the Mean for the percent of maximal response to GIP(1-42)OH.

TABLE 57
Functional cAMP Potency (EC₅₀) and Efficacy (Eₘₐₓ) for compounds
incubated at 37° C. in the presence of 0.1% bovine casein.

| | |
|---|---|
| Example | Human GIP-R |

| Number | EC₅₀, nM^{a} | Eₘₐₓ, %^{b} |
|---|---|---|
| 55 | 0.624 (0.140, n = 2) | 119 (3.88, n = 2) |
| 56 | 0.0532 (0.00576, n = 2) | 126 (7.13, n = 2) |
| 57 | 0.281 (0.0831, n = 2) | 98.7 (3.92, n = 2) |
| 58 | 0.309 (0.111, n = 2) | 108 (0.122, n = 2) |
| 59 | 0.802 (0.0697, n = 2) | 108 (1.49, n = 2) |
| 60 | 12.5 (1.17, n = 2) | 121 (2.19, n = 2) |
| 61 | 23.9 (3.01, n = 2) | 117 (2.92, n = 2) |
| 62 | 1.01 (0.0396, n = 3) | 109 (4.04, n = 3) |
| 63 | 0.915 (0.123, n = 3) | 120 (1.97, n = 3) |
| 64 | 1.88 (0.367, n = 2) | 111 (0.360, n = 2) |
| 65 | 1.89 (0.703, n = 2) | 111 (0.276, n = 2) |
| 66 | 0.967 (0.219, n = 2) | 114 (2.83, n = 2) |
| 67 | 0.369 (0.0413, n = 2) | 111 (4.88, n = 2) |
| 68 | 0.617 (0.0269, n = 3) | 98.0 (3.64, n = 3) |
| 69 | 1.33 (0.0686, n = 2) | 106 (6.00, n = 2) |
| 70 | 1.93 (0.100, n = 2) | 95.8 (5.80, n = 2) |
| 71 | 3.04 (0.549, n = 2) | 91.0 (0.745, n = 2) |
| 72 | 2.04 (0.219, n = 2) | 102 (5.81, n = 2) |
| 73 | 28.4 | 93.5 |
| 74 | 0.723 (0.0182, n = 3) | 101 (3.01, n = 3) |
| 75 | 0.427 (0.0292, n = 3) | 110 (0.669, n = 3) |
| 76 | 0.461 (0.120, n = 3) | 101 (2.10, n = 3) |
| 77 | 2.23 (0.00116, n = 2) | 120 (0.970, n = 2) |
| 78 | 0.427 (0.179, n = 2) | 99.5 (6.08, n = 2) |
| 79 | 0.704 (0.119, n = 2) | 94.6 (0.567, n = 2) |
| 80 | 17.7 (0.270, n = 2) | 117 (6.77, n = 2) |
| 81 | 19.6 (6.23, n = 2) | 101 (5.25, n = 2) |
| 82 | 36.9 | 87.8 |
| 83 | 15.1 (0.385, n = 2) | 82.7 (4.07, n = 2) |
| 84 | 0.601 (0.221, n = 2) | 95.9 (1.36, n = 2) |
| 85 | 28.4 | 121 |
| 86 | 1.54 (0.0526, n = 2) | 95.9 (8.46, n = 2) |
| 87 | 1.70 (0.0417, n = 2) | 102 (7.53, n = 2) |
| 88 | 6.79 (0.224, n = 2) | 98.1 (5.51, n = 2) |
| 89 | 17.1 (0.159, n = 2) | 87.2 (2.19, n = 2) |
| 90 | 24.3 (3.92, n = 2) | 91.2 (0.420, n = 2) |
| 91 | 26.4 | 103 |
| 92 | 93.3 (10.9, n = 2) | 36.3 (0.431, n = 2) |
| 93 | 83.9 | 101 |
| 94 | 1.44 (0.164, n = 2) | 94.8 (3.77, n = 2) |
| 95 | 5.33 (0.0135, n = 2) | 83.0 (3.83, n = 2) |
| 96 | 47.2 | 107 |
| 97 | 2.71 (0.0283, n = 2) | 104 (0.812, n = 2) |
| 98 | 4.29 (1.23, n = 2) | 117 (4.84, n = 2) |
| 99 | 4.28 (0.988, n = 2) | 105 (2.90, n = 2) |
| 100 | 20.9 (2.12, n = 2) | 99.3 (2.32, n = 2) |
| 101 | 30.1 | 72.2 |
| 102 | 0.0967 (0.00283, n = 2) | 93.9 (3.08, n = 2) |
| 103 | 0.615 (0.0383, n = 2) | 96.8 (4.47, n = 2) |
| 104 | 4.83 (0.119, n = 2) | 70.2 (0.222, n = 2) |
| 105 | 19.7 (3.45, n = 2) | 88.5 (2.91, n = 2) |
| 106 | 33.6 (5.51, n = 2) | 86.5 (4.15, n = 2) |
| 107 | 54.4 (0.603, n = 2) | 93.0 (2.47, n = 2) |
| 108 | 79.9 (2.69, n = 2) | 67.2 (0.416, n = 2) |
| 109 | 5.33 (0.348, n = 2) | 109 (7.48, n = 2) |
| 110 | 9.21 (1.36, n = 3) | 113 (1.78, n = 3) |
| 111 | 3.46 (0.316, n = 2) | 49.5 (3.31, n = 2) |
| 112 | 10.7 (2.24, n = 2) | 107 (0.507, n = 2) |
| 113 | 41.4 | 100.0 |
| 114 | 5.12 (0.0243, n = 2) | 100 (0.181, n = 2) |
| 115 | 1.77 (0.193, n = 3) | 101 (4.59, n = 3) |
| 116 | 2.49 (1.03, n = 4) | 101 (1.07, n = 4) |
| 118 | 5.60 (0.237, n = 2) | 67.6 (7.36, n = 2) |
| 119 | 56.5 | 87.7 |
| 120 | 84.0 | 95.8 |
| 121 | 98.5 | 87.8 |

TABLE 58
Functional cAMP Potency (EC₅₀) and Efficacy (Eₘₐₓ) for compounds
incubated at 37° C. in the presence of 0.1% bovine casein

| | |
|---|---|
| Example | Human GIP-R |

| Number | EC₅₀, nM^{a} | Eₘₐₓ, %^{b} |
|---|---|---|
| 122 | 1.16 (0.141, n = 2) | 116 (1.66, n = 2) |
| 123 | 32.0 (10.2, n = 2) | 91.2 (2.57, n = 2) |
| 124 | 140 | 104 |
| 125 | 76.3 | 86.7 |
| 126 | 8.55 (0.254, n = 2) | 90.3 (0.371, n = 2) |
| 127 | 0.496 | 95.4 |
| 128 | 0.412 | 102 |
| 129 | 0.363 | 96.7 |
| 131 | 10.9 | 191 |
| 132 | 1.05 | 103 |
| 133 | 4.20 | 114 |
| 134 | 1.03 | 106 |
| 135 | 4.07 | 118 |
| 136 | 3.44 | 118 |
| 137 | 42.1 | 120 |
| 138 | 0.411 | 115 |
| 139 | 49.0 (11.1, n = 2) | 95.9 (2.06, n = 2) |
| 140 | 45.7 | 110 |
| 141 | 14.2 | 107 |
| 142 | 0.932 (0.0975, n = 3) | 109 (8.11, n = 3) |
| 143 | 0.205 (0.0603, n = 3) | 91.1 (14.3, n = 3) |
| 144 | 1.76 | 111 |
| 145 | 1.04 (0.0644, n = 2) | 136 (3.18, n = 2) |
| 146 | 35.6 (4.00, n = 2) | 107 (3.95, n = 2) |
| 147 | 1.09 | 103 |
| 148 | 0.501 | 100 |
| 149 | 0.529 (0.0418, n = 8) | 112 (3.13, n = 8) |
| 150 | 0.471 | 115 |
| 151 | 0.0626 (0.0326, n = ¾) | 102 (2.92, n = 3) |
| 152 | 1.16 (0.154, n = 3) | 104 (5.96, n = 3) |
| 153 | 0.319 | 110 |
| 154 | 0.283 (0.0348, n = 4) | 105 (4.49, n = 4) |
| 155 | 1.86 (0.0906, n = 2) | 103 (0.574, n = 2) |
| 156 | 0.569 (0.0503, n = 2) | 111 (4.51, n = 2) |
| 157 | 9.96 (1.01, n = 2) | 99.6 (0.321, n = 2) |
| 158 | 0.889 | 98.6 |
| 159 | 10.9 (0.210, n = 2) | 81.9 (3.88, n = 2) |
| 160 | 5.38 (0.276, n = 2) | 113 (4.54, n = 2) |

^{a}EC₅₀, nM = the Geometric Mean with the Standard Error of the Mean followed by the number of observations in parenthesis.
^{b}Eₘₐₓ, % = the Arithmetic Mean ± the Standard Error of the Mean for the percent of maximal response to GIP(1-42)OH.

We claim:

1. A compound of the formula: 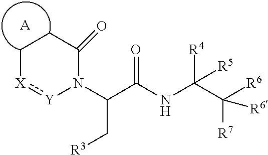
wherein
X is CH₂, OCH₂ or CH₂CH₂, Y is CHR¹⁸ and [symbol] is a single bond, or
X is CH, Y is CR¹⁸ and [symbol] is a double bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond, or
X is CH, Y is N and [symbol] is a double bond;
R¹⁸ is H or CH₃;
A is 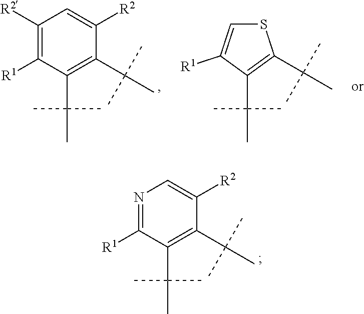
R¹ is an 8-, 9- or 10-membered N-containing bicyclic heterocycle, phenyl or a 5- or 6-membered N-containing heterocycle, wherein the heterocycle or phenyl is optionally substituted with 1 to 3 substituents independently selected from:
CN,
halo,
C₁-C₄alkyl optionally substituted with OCH₃,
C₁-C₄haloalkyl,
C₁-C₄alkoxy,
C₁-C₄haloalkoxy,
CD₃,
oxo,
phenyl optionally substituted with 1 or 2 substituents independently selected from halo and OH,
benzyl optionally substituted with OCH₃,
NHC(O)R¹⁶, and
a 9-membered N-containing bicyclic heterocycle optionally substituted with CH₃:
R¹⁶ is NHC₁-C₄alkyl, C₃-C₆cycloalkyl, NHC₃-C₆cycloalkyl, pyridyl optionally substituted with halo or CH₃, or phenyl optionally substituted with halo;
R² and R^{2′} are independently H, halo or OCH₃;
R³ is:
i) a 5- or 6-membered N-containing heteroaryl optionally substituted with 1 or 2 substituents selected from: CH₃, CD₃, CHF₂, OH and CH₂CN, or
ii) an 8- or 9-membered N-containing bicyclic heterocycle optionally substituted with 1 or 2 substituents selected from oxo, CH₃ and halo:
R⁴ and R⁵ are each CH₃, or together form a cyclopropyl, cyclobutyl, oxetane, tetrahydrofuran, pyrrolidine or piperidine, wherein the cyclopropyl or cyclobutyl is optionally substituted with 1 or 2 halo and wherein the pyrrolidine or piperidine is optionally substituted with CH₃;
R⁶ is H, D, OH, CH₃, CO₂R¹⁰, or 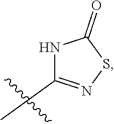
wherein R¹⁰ is H or C₁-C₄alkyl optionally substituted with OC(O)C₁-C₄alkyl or morpholine, R^{6′} is H, and R⁷ is a 5- or 6-membered N-containing heteroaryl, 9-membered N-containing heteroaryl or phenyl, wherein the heteroaryl or phenyl is optionally substituted with R⁸ or with R⁸ and R⁹, or
R⁶ is D, R^{6′} is D and R⁷ is a 5- or 6-membered N-containing heteroaryl, 9-membered N-containing heteroaryl or phenyl, wherein the heteroaryl or phenyl is optionally substituted with R⁸ or with R⁸ and R⁹, or
R⁶ and R⁷ together form a 9-membered N-containing bicyclic heterocycle optionally substituted with 1 to 3 substituents independently selected from: CH₃, CF₃ and oxo, and R^{6′} is H;
R⁸ is CF₃, CF₂H, halo, cyclopropyl, CH₃ or H;
R⁹ is:
i) a 4-, 5- or 6-membered heterocycle optionally substituted with 1 to 4 substituents independently selected from:
halo,
OH,
C₁-C₄alkoxy,
SO₂NH₂,
a 4- to 6-membered heterocycle optionally substituted with CH₃ or oxo,
CN,
C₃-C₆cycloalkyl,
CD₃,
C₁-C₄haloalkyl,
CR¹²R¹³OP(O)(OH)₂
oxo,
C₁-C₄haloalkoxy,
C(O)NR¹²R¹³,
(CH₂)ₘP(O)(R¹⁷)₂, and
C₁-C₄alkyl, wherein the C₁-C₄alkyl is optionally substituted with OH, OCH₃,
NR¹²R¹³, C(O)NR¹²R¹³, CN, a 4- to 6-membered heterocycle or C₃-C₄cycloalkyl optionally substituted with halo,
ii) a 9- or 10-membered N-containing bicyclic heterocycle optionally substituted with 1 or 2 substituents independently selected from CN, oxo, C(O)O(CH₃)₃, OH, CH₃, and halo,
iii) C₁-C₄alkoxy,
iv) C₃-C₅cycloalkyl,
v) halo,
vi) (CH₂)ₙC(O)R¹¹, wherein n is 0 or 1, R¹¹ is OCH₃, NR¹²R¹³ or a 4- to 6-membered heterocycle,
vii) C₁-C₄alkyl optionally substituted with a 4- to 6-membered heterocycle, phenyl, NHS(O)₂(CH₃) or NR¹²R¹³, which heterocycle or phenyl is optionally substituted with CH₃,
viii) CN,
ix) phenyl optionally substituted with 1 to 3 substituents independently selected from C(O)OH, (CH₂)ₘP(O)(R¹⁷)₂, NR¹²R¹³ and C₁-C₄alkyl, wherein the C₁-C₄alkyl is optionally substituted with OH, or
x) (CH₂)ₘP(O)(R¹⁷)₂;
R¹² and R¹³ are independently H or CH₃;
each R¹⁷ is independently C₁-C₄alkyl; and
m is 0 or 1;
or a pharmaceutically acceptable salt thereof.

2. The compound according to claim 1, wherein A is 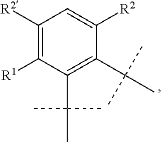
or a pharmaceutically acceptable salt thereof.

3. The compound according to claim 2, wherein R² is F and R^{2′} is H, or a pharmaceutically acceptable salt thereof.

4. The compound according to claim 1, wherein:
X is CH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond, or
X is CH, Y is N and [symbol] is a double bond,
or a pharmaceutically acceptable salt thereof.

5. The compound according to claim 4, wherein:
X is CH₂, Y is CH₂ and [symbol] is a single bond, or a pharmaceutically acceptable salt thereof.

6. The compound according to claim 1, wherein R⁴ and R⁵ are each CH₃, or a pharmaceutically acceptable salt thereof.

7. The compound according to claim 1, wherein R¹ is:
an 8- or 9-membered N-containing bicyclic heterocycle, phenyl or a 6-membered N-containing heterocycle, wherein the heterocycle or phenyl is optionally substituted with 1 to 3 substituents independently selected from:
CN,
halo,
C₁-C₄alkyl,
C₁-C₄haloalkyl,
C₁-C₄alkoxy,
C₁-C₄haloalkoxy,
CD₃,
oxo,
benzyl optionally substituted with OCH₃, and
a 9-membered N-containing bicyclic heterocycle optionally substituted with CH₃, or a pharmaceutically acceptable salt thereof.

8. The compound according to claim 7, wherein R¹ is: 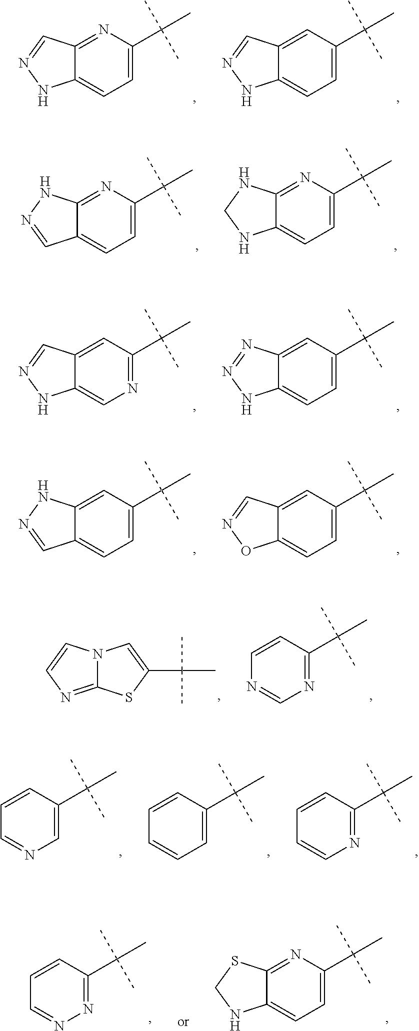
optionally substituted with 1 to 3 substituents independently selected from:
CN,
F,
Cl,
CH₃,
CF₃,
OCH₃,
OCHF₂,
CD₃,
oxo,
benzyl substituted with OCH₃, and
a 9-membered N-containing bicyclic heterocycle substituted with CH₃,
or a pharmaceutically acceptable salt thereof.

9. The compound according to claim 8, wherein R¹ is: 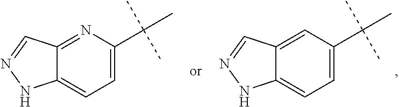
optionally substituted with 1 to 3 substituents independently selected from:
CN,
F, and
CH₃,
or a pharmaceutically acceptable salt thereof.

10. The compound according to claim 1, wherein R³ is a 5- or 6-membered N-containing heteroaryl optionally substituted with CH₃, CD₃ or CHF₂, or a 9-membered N-containing bicyclic heterocycle optionally substituted with 1 or 2 substituents selected from oxo and CH₃, or a pharmaceutically acceptable salt thereof.

11. The compound according to claim 10, wherein R³ is: 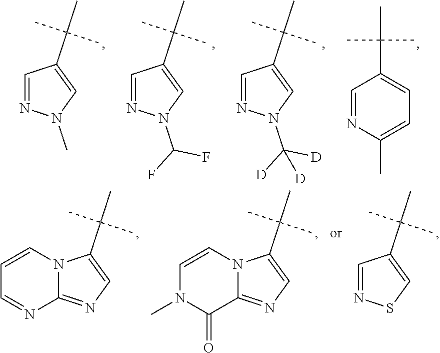
or a pharmaceutically acceptable salt thereof.

12. The compound according to claim 11, wherein R³ is: 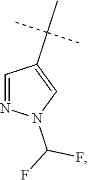
or a pharmaceutically acceptable salt thereof.

13. The compound according to claim 1, wherein R⁶ is H, OH, CO₂R¹⁰, or 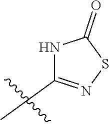 [SMILES: *c1nsc(=O)[nH]1]
and R^{6′} is H, or R⁶ is D and R^{6′} is D, or a pharmaceutically acceptable salt thereof.

14. The compound according to claim 13, wherein R⁶ is H and R^{6′} is H, or a pharmaceutically acceptable salt thereof.

15. The compound according to claim 1, wherein R⁷ is a 5- or 6-membered N-containing heteroaryl, 9-membered N-containing heteroaryl or phenyl, wherein the heteroaryl or phenyl is optionally substituted with R⁸ or with R⁸ and R⁹,
R⁸ is CF₃, halo, cyclopropyl, CH₃ or H; and
R⁹ is:
i) a 4-, 5- or 6-membered heterocycle optionally substituted with 1 to 3 substituents independently selected from:
halo,
OH,
C₁-C₄alkoxy,
a 4- to 6-membered heterocycle,
CN,
C₃-C₆cycloalkyl,
CD₃,
C₁-C₄haloalkyl,
CR¹²R¹³OP(O)(OH)₂
oxo, and
C₁-C₄alkyl, wherein the C₁-C₄alkyl is optionally substituted with OH, NR¹²R¹³, or a 4- to 6-membered heterocycle,
ii) a 9-membered N-containing bicyclic heterocycle,
iii) C₁-C₄alkoxy,
iv) C₃-C₅cycloalkyl,
v) halo,
vi) (CH₂)ₙC(O)R¹¹, wherein n is 0 or 1, R¹¹ is NR¹²R¹³ or a 4- to 6-membered heterocycle,
vii) C₁-C₄alkyl optionally substituted with NHS(O)₂(CH₃), NR¹²R¹³ or phenyl, which phenyl is optionally substituted with CH₃,
viii) CN,
ix) phenyl optionally substituted with 1 or 2 substituents independently selected from C(O)OH, (CH₂)ₘP(O)(R¹⁷)₂ and NR¹²R¹³, or
x) (CH₂)ₘP(O)(R¹⁷)₂;
or a pharmaceutically acceptable salt thereof.

16. The compound according to claim 15, wherein R⁷ is: 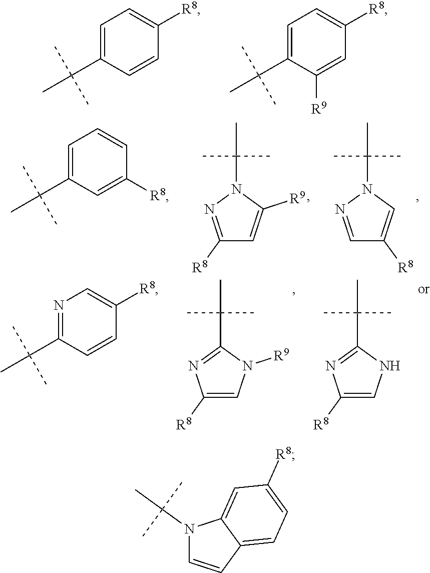
or a pharmaceutically acceptable salt thereof.

17. The compound according to claim 16, wherein R⁷ is 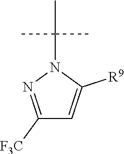
and R⁹ is selected from:

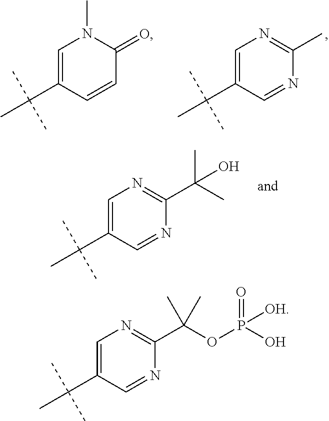

18. The compound according to claim 1 of the formula: 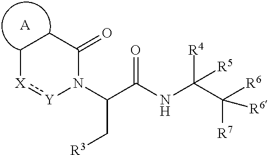
wherein
X is CH₂, OCH₂ or CH₂CH₂, Y is CH₂ and [symbol] is a single bond, or
X is CHR¹⁴, Y is CHR¹⁵, [symbol] is a single bond and R¹⁴ and R¹⁵ together with the carbons to which they are attached form a fused cyclopropyl, or
X is N, Y is CH and [symbol] is a double bond, or
X is CH, Y is N and [symbol] is a double bond;
A is 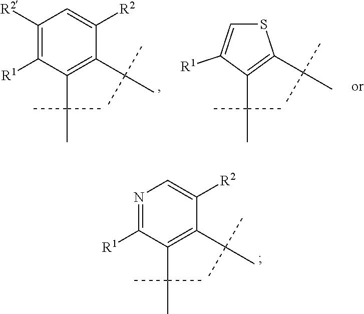
R¹ is an 8-, 9- or 10-membered N-containing bicyclic heterocycle, phenyl or a 5- or 6-membered N-containing heteroaryl, wherein the heterocycle, phenyl or heteroaryl is optionally substituted with 1 to 3 substituents independently selected from:
CN,
halo,
C₁-C₄alkyl optionally substituted with OCH₃,
C₁-C₄haloalkyl,
C₁-C₄alkoxy,
C₁-C₄haloalkoxy,
CD₃,
oxo,
phenyl optionally substituted with 1 or 2 substituents independently selected from halo and OH,
benzyl optionally substituted with OCH₃,
NHC(O)pyridyl, wherein the pyridyl is optionally substituted with halo, and
a 9-membered N-containing bicyclic heterocycle optionally substituted with CH₃;
R² and R^{2′} are independently H, halo or OCH₃;
R³ is a 5- or 6-membered N-containing heteroaryl optionally substituted with 1 or 2 substituents selected from: CH₃, CD₃, CHF₂, OH and CH₂CN, or a 9-membered N-containing bicyclic heterocycle optionally substituted with 1 or 2 substituents selected from oxo and CH₃;
R⁴ and R⁵ are each CH₃ or together form a cyclopropyl;
R⁶ is H, D, OH, CH₃, CO₂R¹⁰, or 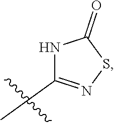
wherein R¹⁰ is H or C₁-C₄alkyl optionally substituted with OC(O)C₁-C₄alkyl or morpholine, and R^{6′} is H, or
R⁶ is D and R^{6′} is D;
R⁷ is a 5- or 6-membered N-containing heteroaryl or phenyl, wherein the heteroaryl or phenyl is optionally substituted with R⁸ or with R⁸ and R⁹, or wherein the phenyl is optionally substituted with 1 or 2 substituents selected from: halo, CF₃, and pyrazine optionally substituted with CF₂H or OCH₃;
R⁸ is CF₃, CF₂H, halo or cyclopropyl;
R⁹ is:
i) a 5- or 6-membered heterocycle optionally substituted with 1 to 3 substituents independently selected from:
halo,
OH,
C₁-C₄alkoxy,
SO₂NH₂,
a 4- to 6-membered heterocycle optionally substituted with CH₃,
CN,
C₃-C₆cycloalkyl,
CD₃,
C₁-C₄haloalkyl,
CR¹²R¹³OP(O)(OH)₂
oxo, and
C₁-C₄alkyl, wherein the C₁-C₄alkyl is optionally substituted with OH, OCH₃, N(CH₃)₂, a 4- to 6-membered heterocycle or C₃-C₄cycloalkyl optionally substituted with halo,
ii) a 9-membered N-containing bicyclic heterocycle optionally substituted with CN, oxo, C(O)O(CH₃)₃ or OH,
iii) C₁-C₄alkoxy,
iv) C₃-C₅cycloalkyl,
v) halo,
vi) (CH₃)ₙC(O)R¹¹, wherein n is 0 or 1, R¹¹ is OCH₃, NR¹²R¹³ or a 4- to 6-membered heterocycle,
vii) C₁-C₄alkyl optionally substituted with a 4- to 6-membered heterocycle or phenyl, which heterocycle or phenyl is optionally substituted by CH₃,
viii) CN, or
ix) phenyl optionally substituted with C(O)OH; and
R¹² and R¹³ are independently H or CH₃;
or a pharmaceutically acceptable salt thereof.

19. The compound according to claim 1 of the formula: 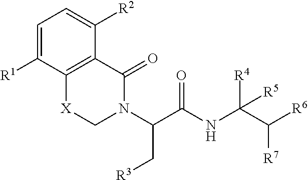
wherein
X is —CH₂—, —OCH₂— or —CH₂CH₂—;
R¹ is:
i) an 8-, 9- or 10-membered N-containing bicyclic heterocycle optionally substituted with 1 to 3 substituents independently selected from:
CN,
halo,
C₁-C₄alkyl optionally substituted with OCH₃,
C₁-C₄haloalkyl,
oxo,
phenyl optionally substituted with 1 or 2 substituents independently selected from halo and OH, and
benzyl optionally substituted with OCH₃,
ii) phenyl or pyridyl optionally substituted with 1 or 2 substituents independently selected from:
CN,
NHC(O)pyridyl, wherein the pyridyl is optionally substituted with halo, and a 9-membered N-containing bicyclic heterocycle optionally substituted with CH₃, or
iii) pyrazolyl optionally substituted with 1 to 3 substituents selected from CN and CH₃;
R² is H, halo or OCH₃;
R³ is a 5- or 6-membered N-containing heteroaryl optionally substituted with 1 or 2 substituents selected from: CH₃, CHF₂, OH and CH₂CN;
R⁴ and R⁵ are each CH₃ or together form a cyclopropyl;
R⁶ is H, OH, CH₃, CO₂R¹⁰, or 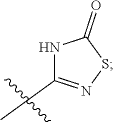 [SMILES: *c1nsc(=O)[nH]1]
R⁷ is:
i) phenyl optionally substituted with 1 or 2 substituents selected from:
halo,
CF₃, and
pyrazine optionally substituted with CF₂H or OCH₃, or
ii) a 5- or 6-membered N-containing heteroaryl optionally substituted with R⁸ or with R⁸ and R⁹;
R⁸ is CF₃, CF₂H, halo or cyclopropyl;
R⁹ is:
i) a 5- or 6-membered heterocycle optionally substituted with 1 to 3 substituents independently selected from:
halo,
OH,
OCH₃,
SO₂NH₂,
a 4- to 6-membered heterocycle,
oxo, and
C₁-C₄alkyl, wherein the C₁-C₄alkyl is optionally substituted with OH, OCH₃, a 4-to 6-membered heterocycle or C₃-C₄cycloalkyl optionally substituted with halo,
ii) a 9-membered N-containing bicyclic heterocycle optionally substituted with CN, oxo, C(O)O(CH₃)₃ or OH,
iii) C₁-C₄alkoxy,
iv) C₃-C₅cycloalkyl,
v) halo, or
vi) C(O)OCH₃; and
R¹⁰ is H or C₁-C₄alkyl optionally substituted with OC(O)C₁-C₄alkyl or morpholine,
or a pharmaceutically acceptable salt thereof.

20. The compound according to claim 1, wherein the compound is selected from Examples 1 to 160, a pharmaceutically acceptable salt thereof.

21. A pharmaceutical composition comprising a compound, or a pharmaceutically acceptable salt thereof, according to claim 1 and at least one pharmaceutically acceptable carrier, diluent, or excipient.

22. A method for treating type II diabetes mellitus comprising administering to a patient in need thereof a therapeutically effective amount of a compound according to claim 1, or a pharmaceutically acceptable salt thereof.

23. A method for treating obesity comprising administering to a patient in need thereof a therapeutically effective amount of a compound according to claim 1, or a pharmaceutically acceptable salt thereof.

24.-26. (canceled)
